(19) United States
(12) Patent Application Publication
(10) Pub. No.: US 2024/0228512 A1
(43) Pub. Date: Jul. 11, 2024

(54) ACYCLIC OXAZEPINE COMPOUNDS COMPRISING A 6-AZA MOIETY AND USES THEREOF

(71) Applicant: Genentech, Inc., South San Francisco, CA (US)

(72) Inventors: Benjamin David RAVETZ, Burlingame, CA (US); Jack Alexander TERRETT, San Francisco, CA (US); BinQing WEI, San Carlos, CA (US); Mingshuo ZENG, Daly City, CA (US); Cheng SHAO, Beijing (CN); Yinlei SUN, Beijing (CN)

(73) Assignee: Genentech, Inc., South San Francisco, CA (US)

(21) Appl. No.: 18/490,736

(22) Filed: Oct. 19, 2023

(30) Foreign Application Priority Data
Oct. 19, 2022 (WO) PCT/CN2022/126190
Oct. 18, 2023 (WO) PCT/CN2023/125292

(51) Int. Cl. C07D 519/00; A61K 31/553; A61P 31/00; C07D 498/16

(52) U.S. Cl. CPC C07D 519/00; A61K 31/553; A61P 31/00; C07D 498/16

(57) ABSTRACT

Provided herein are acyclic oxazepinyl compounds comprising a 6-aza moiety that are useful in the treatment of cancers.

### TECHNICAL FIELD

Provided herein are acyclic compounds comprising a 6-aza moiety that are useful in the treatment of cancers comprising a KRas mutation, compositions of such compounds, and methods of treating cancers comprising a KRas mutation.

### BACKGROUND

Ras is a small GTP-binding protein that functions as a nucleotide-dependent switch for central growth signaling pathways. In response to extracellular signals, Ras is converted from a GDP-bound (Ras^{GDP}) to a GTP-bound (Ras^{GTP}) state, as catalyzed by guanine nucleotide exchange factors (GEFs), notably the SOS1 protein. Active RaS^{GTP} mediates its diverse growth-stimulating functions through its direct interactions with effectors including Raf, PI3K, and Ral guanine nucleotide dissociation stimulator. The intrinsic GTPase activity of Ras then hydrolyzes GTP to GDP to terminate Ras signaling. The Ras GTPase activity can be further accelerated by its interactions with GTPase-activating proteins (GAPs), including the neurofibromin 1 tumor suppressor.

Mutant Ras has a reduced GTPase activity, which prolongs its activated state, thereby promoting Ras-dependent signaling and cancer cell survival or growth. Mutation in Ras that affects its ability to interact with GAP or to convert GTP back to GDP will result in a prolonged activation of the protein and consequently a prolonged signal to the cell telling it to continue to grow and divide. Because these signals result in cell growth and division, overactive RAS signaling may ultimately lead to cancer. Mutations in any one of the three main isoforms of RAS (HRas, NRas, or KRas) genes are common events in human tumorigenesis. Among the three Ras isoforms (K, N, and H), KRas is most frequently mutated.

The most common KRas mutations are found at residue G12 and G13 in the P-loop and at residue Q61. Mutations of Ras in cancer are associated with poor prognosis. Inactivation of oncogenic Ras in mice results in tumor shrinkage. Thus, Ras is widely considered an oncology target of exceptional importance.

Accordingly, there is a pressing need for therapies for mutant KRas mediated cancers.

### SUMMARY

Provided herein are solutions to the problems above and other problems in the art.

In one aspect, provided herein are compounds or an atropisomer, stereoisomer, tautomer, or pharmaceutically acceptable salt thereof of formula (I) as described herein.

In another aspect, provided herein are compounds or an atropisomer, stereoisomer, tautomer, or pharmaceutically acceptable salt thereof of formula (Ia) as described herein.

In another aspect, provided herein are compounds or an atropisomer, stereoisomer, tautomer, or pharmaceutically acceptable salt thereof of formula (II), (IIa), (IIb), or (IIc) as described herein.

In another aspect, provided herein are compounds or an atropisomer, stereoisomer, tautomer, or pharmaceutically acceptable salt thereof of formula (III), (IIIa), (IIIb), or (IIIc) as described herein.

In another aspect, provided herein are compounds or an atropisomer, stereoisomer, tautomer, or pharmaceutically acceptable salt thereof of formula (IV), (IVa), (IVb), or (IVc) as described herein.

In another aspect, provided herein are compounds or an atropisomer, stereoisomer, tautomer, or pharmaceutically acceptable salt thereof of formula (V), (Va), (Vb), or (Vc) as described herein.

In another aspect, provided herein are pharmaceutical compositions comprising a compound or an atropisomer, stereoisomer, tautomer, or pharmaceutically acceptable salt thereof as described herein and one or more pharmaceutically acceptable excipients.

In another aspect, provided herein are methods of treating cancer as described herein by administering an effective amount of a compound or an atropisomer, stereoisomer, tautomer, or pharmaceutically acceptable salt thereof described herein.

### DETAILED DESCRIPTION

Disclosed herein are acyclic oxazepine compounds as described herein or a stereoisomer, atropisomer, tautomer, or pharmaceutically acceptable salt thereof and pharmaceutical compositions thereof that, in certain embodiments, are inhibitors or modulators of mutant KRas. In certain instances, such compounds and compositions are inhibitors or modulators of mutant KRas (i.e. pan-KRas inhibitors) as provided herein. The compounds and compositions described herein are useful in treating diseases and disorders mediated by mutant KRas.

While the disclosure herein provides enumerated embodiments, it is understood that they are not intended to limit the compounds and methods described herein to those embodiments. On the contrary, the disclosure is intended to cover all alternatives, modifications, and equivalents that can be included within the scope of the present disclosure as defined by the claims.

Unless otherwise defined, all technical and scientific terms used herein have the same meaning as commonly understood by one of ordinary skill in the art. All publications, patent applications, patents, and other references mentioned herein are incorporated by reference in their entirety. The nomenclature used in this Application is based on IUPAC systematic nomenclature, unless indicated otherwise.

The following definitions are provided to facilitate understanding of certain terms used frequently herein and are not meant to limit the scope of the present disclosure. All references referred to herein are incorporated by reference in their entirety.

The terms “halogen” and “halo” are used interchangeably and refer to F, Cl, Br or I. Additionally, terms such as “haloalkyl,” are meant to include monohaloalkyl, polyhaloalkyl, and perhaloalkyl.

The term “alkyl” refers to a saturated linear or branched-chain monovalent hydrocarbon radical. In one example, the alkyl radical is one to eighteen carbon atoms (C₁₋₁₈). In other examples, the alkyl radical is C₁₋₁₂, C₁₋₁₀, C₁₋₈, C₁₋₆, C₁₋₅, C₁₋₄, or C₁₋₃. Examples of alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl and 1-octyl.

The term “oxo” refers to ═O.

The term “alkoxy” refers to —O-alkyl.

The terms “cyano” or “nitrile” refers to —C≡N or −CN.

The term “haloalkoxy” refers to —O-haloalkyl.

The terms “hydroxy” and “hydroxyl” refer to —OH.

The term “alkylidene” refers to linear or branched-chain monovalent hydrocarbon radical having formula=CR′R″, where R′ and R′ can be the same or different. In one example, an alkylidene radical is 1 to 6 carbons (C₁₋₆). In another example, the alkylidene radical is C₁₋₃, C₁₋₂, or C₁. Examplary alkylidenes include, but are not limited to, methylidene (═CH₂), ethylidene (═CHCH₃), and propylidene (═CH—CH₂—CH₃).

The term “alkenyl” refers to linear or branched-chain monovalent hydrocarbon radical with at least one carbon-carbon double bond, and includes radicals having “cis” and “trans” orientations, or alternatively, “E” and “Z” orientations. In one example, the alkenyl radical is two to eighteen carbon atoms (C₂₋₁₈). In other examples, the alkenyl radical is C₂₋₁₂, C₂₋₁₀, C₂₋₈, C₂₋₆, or C₂₋₃. Examples include, but are not limited to, ethenyl or vinyl (—CH═CH₂), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl.

The term “alkynyl” refers to a linear or branched monovalent hydrocarbon radical with at least one carbon-carbon, triple bond. In one example, the alkynyl radical is two to eighteen carbon atoms (C₂₋₁₈). In other examples, the alkynyl radical is C₂₋₁₂, C₂₋₁₀, C₂₋₈, C₂₋₆, or C₂₋₃. Examples include, but are not limited to, ethynyl (—C≡CH), prop-1-ynyl (—C≡CCH₃), prop-2-ynyl (propargyl, —CH₂C≡CH), but-1-ynyl, but-2-ynyl, and but-3-ynyl.

The term “alkylene” refers to a saturated, branched, or straight chain hydrocarbon group having two monovalent radical centers derived by the removal of two hydrogen atoms from the same or two different carbon atoms of a parent alkane. In one example, the divalent alkylene group is one to eighteen carbon atoms (C₁₋₁₈). In other examples, the divalent alkylene group is C₁₋₁₂, C₁₋₁₀, C₁₋₈, C₁₋₆, C₁₋₅, C₁₋₄, or C₁₋₃. Example 6alkylene groups include methylene (—CH₂—), 1,1-ethyl (—CH(CH₃)—), (1,2-ethyl (—CH₂CH₂—), 1,1-propyl (—CH(CH₂CH₃)—), 2,2-propyl (—C(CH₃)₂—), 1,2-propyl (—CH(CH₃)CH₂—), 1,3-propyl (—CH₂CH₂CH₂—), 1,1-dimethyleth-1,2-yl (—C(CH₃)₂CH₂—), 1,4-butyl (—CH₂CH₂CH₂CH₂—), and the like.

The term “cycloalkyl” refers to a saturated hydrocarbon ring group. Cycloalkyl encompasses mono-, bi-, tricyclic, spiro and bridged, saturated ring systems. In one example, the cycloalkyl group is 3 to 12 carbon atoms (C₃₋₁₂). In other examples, cycloalkyl is C₃₋₄, C₃₋₅, C₃₋₇, C₃₋₈, C₃₋₁₀, or C₅₋₁₀. In other examples, the cycloalkyl group, as a monocycle, is C₃₋₄, C₃₋₈, C₃₋₆, or 05-6. In another example, the cycloalkyl group, as a bicycle, is C₇-C₁₂. In another example, the cycloalkyl group, as a spiro system, is C₅₋₁₂. Examples of monocyclic cycloalkyl include cyclopropyl, cyclobutyl, cyclopentyl, cyclohexyl, cycloheptyl, cyclooctyl, cyclononyl, cyclodecyl, cycloundecyl and cyclododecyl. Exemplary arrangements of bicyclic cycloalkyls having 7 to 12 ring atoms include, but are not limited to, [4,4], [4,5], [5,5], [5,6] or [6,6] ring systems. Exemplary bridged bicyclic cycloalkyls include, but are not limited to, bicyclo[2.2.1]heptane, bicyclo[2.2.2]octane and bicyclo[3.2.2]nonane. Examples of spirocycloalkyl include, spiro[2.2]pentane, spiro[2.3]hexane, spiro[2.4]heptane, spiro[2.5]octane and spiro[4.5]decane.

The terms “heterocyclic group”, “heterocyclic”, “heterocycle”, “heterocyclyl”, or “heterocyclo” are used interchangeably and refer to any mono-, bi-, tricyclic, spiro or bridged, saturated, partially saturated or unsaturated, non-aromatic ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocycle, regardless of the point of attachment of the cyclic system to the rest of the molecule. In one example, heterocyclyl includes 3-10 ring atoms (“members”) and includes monocycles, bicycles, tricycles, spiro, and bridged ring systems, wherein the ring atoms are carbon, where at least one atom in the ring or ring system is a heteroatom selected from nitrogen, sulfur or oxygen. In other examples, heterocyclyl includes 3-6, 5-9, 4-10 or 5-10 ring atoms. In one example, heterocyclyl includes 1 to 4 heteroatoms. In one example, heterocyclyl includes 1 to 3 heteroatoms. In another example, heterocyclyl includes 3- to 7-membered monocycles having 1-2, 1-3 or 1-4 heteroatoms selected from nitrogen, sulfur or oxygen. In another example, heterocyclyl includes 4- to 6-membered monocycles having 1-2, 1-3 or 1-4 heteroatoms selected from nitrogen, sulfur or oxygen. In another example, heterocyclyl includes 3-membered monocycles. In another example, heterocyclyl includes 4-membered monocycles. In another example, heterocyclyl includes 5-6 membered monocycles. In another example, heterocyclyl includes 8, 9, or 10 membered bicycles. In such examples, the heterocyclyl group can be 4,5-, 5,5-, 4,6-, 5,6-, or 6,6-fused ring system. In some embodiments, a heterocycloalkyl includes at least one nitrogen. In one example, the heterocyclyl group includes 0 to 3 double bonds. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Example 6heterocycles are oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydrofuranyl, dihydrothienyl, tetrahydrothienyl, imidazolidinyl, piperidinyl, piperazinyl, isoquinolinyl, tetrahydroisoquinolinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, 1,1-dioxoisothiazolyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-onyl, azaspiro[5.5]undecanyl, tetrahydroindolyl, octahydroindolyl, tetrahydroisoindolyl, tetrahydroindazolyl, 1,1-dioxohexahydrothiopyranyl.

“Aryl” as used herein refers to an unsaturated aromatic carbocyclic group having a single ring (e.g., phenyl) or multiple fused or spiro rings (e.g., naphthyl or anthryl) which fused or spiro rings can or can not be aromatic. Particular aryl groups are those having from 6 to 14 annular (i.e., ring) carbon atoms (a “C₆₋₁₄ aryl”). Preferred aryl groups include those having 5 to 6 ring carbons. An aryl group having more than one ring where at least one ring is non-aromatic can be connected to the parent structure at either an aromatic ring position or at a non-aromatic ring position. In one variation, an aryl group having more than one ring where at least one ring is non-aromatic is connected to the parent structure at an aromatic ring position.

The term “heteroaryl” refers to any mono- or bicyclic aromatic ring system containing from 1 to 4 heteroatoms selected from nitrogen, oxygen, and sulfur, and in an Example 6embodiment, at least one heteroatom is nitrogen. Included are any bicyclic groups where any of the above heteroaryl rings are fused to an aryl ring, wherein the aryl ring or the heteroaryl ring is joined to the remainder of the molecule. A heteroaryl group can have a single ring (e.g., pyridyl, furyl) or multiple fused or spiro rings (e.g., indolizinyl, benzothienyl) which fused or spiro rings can or can not be aromatic. In one embodiment, heteroaryl includes 5-6 membered monocyclic aromatic groups where one or more ring atoms is nitrogen, sulfur or oxygen. Example 6heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, tetrazolo[1,5-b]pyridazinyl, imidazol[1,2-a]pyrimidinyl and purinyl, as well as benzo-fused derivatives, for Example 6benzoxazolyl, benzofuryl, benzothiazolyl, benzothiadiazolyl, benzotriazolyl, benzoimidazolyl, indazolyl and indolyl.

In particular embodiments, a heterocyclylgroup or a heteroaryl group is attached at a carbon atom of the heterocyclyl group or the heteroaryl group. By way of example, carbon bonded heterocyclyl groups include bonding arrangements at position 2, 3, 4, 5, or 6 of a pyridine ring, position 3, 4, 5, or 6 of a pyridazine ring, position 2, 4, 5, or 6 of a pyrimidine ring, position 2, 3, 5, or 6 of a pyrazine ring, position 2, 3, 4, or 5 of a furan, tetrahydrofuran, thiofuran, thiophene, pyrrole or tetrahydropyrrole ring, position 2, 4, or 5 of an oxazole, imidazole or thiazole ring, position 3, 4, or 5 of an isoxazole, pyrazole, or isothiazole ring, position 2 or 3 of an aziridine ring, position 2, 3, or 4 of an azetidine ring, position 2, 3, 4, 5, 6, 7, or 8 of a quinoline ring or position 1, 3, 4, 5, 6, 7, or 8 of an isoquinoline ring.

In certain embodiments, the heterocyclyl group or heteroaryl group is N-attached. By way of example, nitrogen bonded heterocyclyl or heteroaryl groups include bonding arrangements at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

“Fused” refers to any ring structure described herein that shares one or more atoms (e.g., carbon or nitrogen atoms) with an existing ring structure in the compounds described herein.

The term “haloalkyl” refers to an alkyl chain in which one or more hydrogen has been replaced by a halogen. Examples of haloalkyls are trifluoromethyl, difluoromethyl, and fluoromethyl. A substituted haloalkyl refers to a haloalkyl having a moiety other than a halogen.

As used herein a wavy line “ [symbol] ” that intersects a bond in a chemical structure indicate the point of attachment of the atom to which the wavy bond is connected in the chemical structure to the remainder of a molecule, or to the remainder of a fragment of a molecule.

In certain embodiments, divalent groups are described generically without specific bonding configurations. It is understood that the generic description is meant to include both bonding configurations, unless specified otherwise. For example, in the group R¹—R²-R³, if the group R² is described as —CH₂C(O)—, then it is understood that this group can be bonded both as R¹—CH₂C(O)—R³, and as R¹—C(O)CH₂-R³, unless specified otherwise.

The term “pharmaceutically acceptable” refers to molecular entities and compositions that do not produce an adverse, allergic or other untoward reaction when administered to an animal, such as, for example, a human, as appropriate.

Compounds described herein may be in the form of a salt, such as a pharmaceutically acceptable salt. “Pharmaceutically acceptable salts” include both acid and base addition salts. “Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, benzenesulfonic acid, p-toluenesulfonic acid, salicylic acid and the like.

The term “pharmaceutically acceptable base addition salts” include those derived from inorganic bases such as sodium, potassium, lithium, ammonium, calcium, magnesium, iron, zinc, copper, manganese, aluminum salts and the like. Particular base addition salts are the ammonium, potassium, sodium, calcium and magnesium salts.

Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particular organic non-toxic bases include isopropylamine, diethylamine, ethanolamine, tromethamine, dicyclohexylamine, choline, and caffeine.

In some embodiments, a salt is selected from a hydrochloride, hydrobromide, trifluoroacetate, sulfate, phosphate, acetate, fumarate, maleate, tartrate, lactate, citrate, pyruvate, succinate, oxalate, methanesulfonate, p-toluenesulfonate, bisulfate, benzenesulfonate, ethanesulfonate, malonate, xinafoate, ascorbate, oleate, nicotinate, saccharinate, adipate, formate, glycolate, palmitate, L-lactate, D-lactate, aspartate, malate, L-tartrate, D-tartrate, stearate, furoate (e.g., 2-furoate or 3-furoate), napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isothionate (2-hydroxyethylsulfonate), 2-mesitylenesulfonate, 2-naphthalenesulfonate, 2,5-dichlorobenzenesulfonate, D-mandelate, L-mandelate, cinnamate, benzoate, adipate, esylate, malonate, mesitylate (2-mesitylenesulfonate), napsylate (2-naphthalenesulfonate), camsylate (camphor-10-sulfonate, for Example 6(1 S)-(+)-10-camphorsulfonic acid salt), glutamate, glutarate, hippurate (2-(benzoylamino)acetate), orotate, xylate (p-xylene-2-sulfonate), and pamoic (2,2′-dihydroxy-1,1′-dinaphthylmethane-3,3′-dicarboxylate).

The term “stereoisomers” refer to compounds that have identical chemical constitution, but differ with regard to the arrangement of the atoms or groups in space. Stereoisomers include diastereomers, enantiomers, atropisomers, conformers and the like.

The term “chiral” refers to molecules that have the property of non-superimposability of the mirror image partner, while the term “achiral” refers to molecules which are superimposable on their mirror image partner.

The term “diastereomer” refers to a stereoisomer with two or more centers of chirality and whose molecules are not mirror images of one another. Diastereomers have different physical properties, e.g., melting points, boiling points, spectral properties or biological activities. Mixtures of diastereomers may separate under high resolution analytical procedures such as electrophoresis and chromatography such as HPLC.

The term “enantiomers” refers to two stereoisomers of a compound that are non-superimposable mirror images of one another.

The term “atropisomers” refers to two conformers resulting from hindered rotation about a single bond where the steric strain barrier to rotation can be high enough to allow for the isolation of the each conformer.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and I or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies that are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Certain compounds described herein can exist in unsolvated forms as well as solvated forms, including hydrated forms. A “solvate” refers to an association or complex of one or more solvent molecules and a compound described herein. Examples of solvents that form solvates include water, isopropanol, ethanol, methanol, DMSO, ethyl acetate, acetic acid, and ethanolamine. Certain compounds described herein can exist in multiple crystalline or amorphous forms. In general, all physical forms are contemplated herein. The term “hydrate” refers to the complex where the solvent molecule is water.

The compounds and pharmaceutically acceptable salts thereof described herein also embrace isotopically-labeled compounds that are identical to those recited herein, but for the fact that one or more atoms are replaced by an atom having an atomic mass or mass number different from the atomic mass or mass number usually found in nature. All isotopes of any particular atom or element as specified are contemplated herein, and their uses. Exemplary isotopes that can be incorporated into compounds and pharmaceutically acceptable salts thereof described herein include isotopes of hydrogen, carbon, nitrogen, oxygen, phosphorus, sulfur, fluorine, chlorine and iodine, such as ²H, ³H, ¹¹C, ¹³C ¹⁴C ¹³N, ¹⁵N, ¹⁵O, ¹⁷O, ¹⁸, O³²P, ³³P, ³⁵S, ¹⁸F, ³⁶C, ¹²³I, and ¹²⁵I. Certain isotopically-labeled compounds or pharmaceutical acceptable salts thereof described herein (e.g., those labeled with ³H and ¹⁴C) are useful in compound and/or substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds or pharmaceutical acceptable salts thereof described herein can generally be prepared by following procedures analogous to those disclosed in the Examples herein below, by substituting an isotopically labeled reagent for a non-isotopically labeled reagent.

Compounds and pharmaceutically acceptable salts thereof described herein may contain one or more asymmetric carbon atoms. Accordingly, the compounds may exist as hdiastereomers, enantiomers or mixtures thereof. The syntheses of the compounds may employ racemates, diastereomers or enantiomers as starting materials or as intermediates. Mixtures of particular diastereomeric compounds may be separated, or enriched in one or more particular diastereomers, by chromatographic or crystallization methods. Similarly, enantiomeric mixtures may be separated, or enantiomerically enriched, using the same techniques or others known in the art. Each of the asymmetric carbon or nitrogen atoms may be in the R or S configuration and both of these configurations are contemplated herein.

In the structures shown herein, where the stereochemistry of any particular chiral atom is not specified, then all stereoisomers are contemplated and included. Where stereochemistry is specified by a solid wedge or dashed line representing a particular configuration, then that stereoisomer is so specified and defined. Unless otherwise specified, if solid wedges or dashed lines are used, relative stereochemistry is intended.

A “subject,” “individual,” or “patient” is a vertebrate and are used interchangeably herein. In certain embodiments, the vertebrate is a mammal. Mammals include, but are not limited to, farm animals (such as cows), sport animals, pets (such as guinea pigs, cats, dogs, rabbits and horses), primates, mice and rats. In certain embodiments, a mammal is a human. In embodiments comprising administration of a compound of to a patient, the patient is typically in need thereof.

The terms “inhibiting” and “reducing,” or any variation of these terms, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity compared to normal.

The term “treatment” refers to clinical intervention designed to alter the natural course of the patient or cell being treated during the course of clinical pathology. Desirable effects of treatment include decreasing the rate of disease progression, ameliorating or palliating the disease state, and remission or improved prognosis. For example, a patient is successfully “treated” if one or more symptoms associated with a cancer described herein are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of patients.

The term “delaying progression” of a disease refers to deferring, hindering, slowing, retarding, stabilizing, and/or postponing development of a cancer described herein. This delay can be of varying lengths of time, depending on the history of the cancer and/or patient being treated. As is evident to one skilled in the art, a sufficient or significant delay can, in effect, encompass prevention, in that the patient does not develop cancer or relapse.

A “mutant KRas mediated disease” and the like refer to a disease described herein (e.g. a cancer described herein) having symptoms or requiring treatment as set forth herein that is/are wholly or partly associated with, a result of, a function of, or otherwise correlated to mutant KRas activity as described herein. In one embodiment, the mutant KRas is any G12 mutant of KRas.

The term “pan-KRas” as used herein refers to an inhibitor as described herein that inhibits the activity of a mutant KRas protein. In some embodiments, the mutant KRas protein comprises a mutation corresponding to the 12 position (G12).

A “KRasG12X mutation” refers to a mutant KRas protein having a mutation corresponding to the position of Gly12. In some embodiments, the mutation corresponds to G12A, G12C, G12D, G12R, G12S, G12V. In another embodiment, the mutation corresponds to G12C, G12D, G12R, or G12V.

An “effective amount” or “therapeutically effective amount” is at least the minimum amount required to effect a measurable improvement or prevention of a cancer described herein. An effective amount herein may vary according to factors such as the disease state, age, sex, and weight of the patient, and the ability of the agent to elicit a desired response in the patient. An effective amount is also one in which any toxic or detrimental effects of the treatment are outweighed by the therapeutically beneficial effects. Beneficial or desired results include results such as eliminating or reducing the risk, lessening the severity, delaying the onset of the disease (including biochemical, histological and/or behavioral symptoms of the disease, its complications and intermediate pathological phenotypes presenting during development of the disease), decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In some embodiments, an effective amount of the drug may have the effect in reducing the number of cancer cells; reducing the tumor size; inhibiting (i.e., slow or stop) cancer cell infiltration into peripheral organs; inhibit (i.e., slow or stop) tumor metastasis; inhibiting (i.e., slow or stop) tumor growth; and/or relieving one or more of the symptoms associated with the disorder. An effective amount can be administered in one or more administrations.

An “administration period” or “cycle” refers to a period of time comprising administration of one or more compounds or pharmaceutically acceptable salts thereof described herein or an additional therapeutic agent (i.e. a chemotherapeutic agent) and an optional period of time comprising no administration of one or more of agents or compounds described herein. A “rest period” refers to a period of time where at least one of agent or compound described herein is not administered. In one embodiment, a rest period refers to a period of time where no agent or compound described herein is administered. A rest period as provided herein can in some instances include administration of an additional agent in the absence of a compound or pharmaceutically acceptable salt thereof described herein or vice versa. In such instances, administration of any agent during a rest period should not interfere or detriment administration of a compound or pharmaceutically acceptable salt thereof described herein.

A “dosing regimen” refers to a period of administration of a compound or pharmaceutically acceptable salt thereof described herein comprising one or more cycles, where each cycle can include administration of a compound or pharmaceutically acceptable salt thereof described herein at different times or in different amounts.

“QD” refers to administration of a compound or pharmaceutically acceptable salt thereof once daily.

“BID” refers to administration of a compound or pharmaceutically acceptable salt thereof twice a day.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times (i.e. sequential administration) in separate compositions, or administration in a composition in which both agents are present.

A “1 L therapy” refers to the first line therapy administered to a treatment naïve cancer patient. Likewise, a 2L, 3L, and the like refer to subsequent therapies administered to a patient.

The term “package insert” is used to refer to instructions customarily included in commercial packages of therapeutic products, that contain information about the indications, usage, dosage, administration, contraindications and/or warnings concerning the use of such therapeutic products.

The terms “antagonist” and “inhibitor” are used interchangeably, and they refer to a compound having the ability to inhibit a biological function of a target protein, whether by inhibiting the activity or expression of the protein, such as a mutant form of KRas. Accordingly, the terms “antagonist” and “inhibitors” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g., bind to) the target, compounds that initiate or enhance a biological activity of the target polypeptide by interacting with other members of the signal transduction pathway of which the target polypeptide is a member are also specifically included within this definition.

The terms “cancer” and “cancerous”, “neoplasm”, and “tumor” and related terms are used interchangeably herein and refer to or describe the physiological condition in mammals that is typically characterized by unregulated cell growth. A “tumor” comprises one or more cancerous cells. Examples of cancer include carcinoma, blastoma, sarcoma, seminoma, glioblastoma, melanoma, leukemia, and myeloid or lymphoid malignancies. More particular examples of such cancers include squamous cell cancer (e.g., epithelial squamous cell cancer) and lung cancer including small-cell lung cancer, non-small cell lung cancer (“NSCLC”), adenocarcinoma of the lung and squamous carcinoma of the lung. Other cancers include skin, keratoacanthoma, follicular carcinoma, hairy cell leukemia, buccal cavity, pharynx (oral), lip, tongue, mouth, salivary gland, esophageal, larynx, hepatocellular, gastric, stomach, gastrointestinal, small intestine, large intestine, pancreatic, cervical, ovarian, liver, bladder, hepatoma, breast, colon, rectal, colorectal, genitourinary, biliary passage, thyroid, papillary, hepatic, endometrial, uterine, salivary gland, kidney or renal, prostate, testis, vulval, peritoneum, anal, penile, bone, multiple myeloma, B-cell lymphoma, diffuse large B-Cell lymphoma (DLBCL), central nervous system, brain, head and neck, Hodgkin's, and associated metastases. Other examples of neoplastic disorders include myeloproliferative disorders, such as polycythemia vera, essential thrombocytosis, myelofibrosis, such as primary myelofibrosis, and chronic myelogenous leukemia (CML).

A “chemotherapeutic agent” is an agent useful in the treatment of a given disorder, for example, cancer or inflammatory disorders. Examples of chemotherapeutic agents are well-known in the art. Additionally, chemotherapeutic agents include pharmaceutically acceptable salts, acids or derivatives of any of chemotherapeutic agents, as well as combinations of two or more of them.

It is specifically contemplated that any limitation discussed with respect to one embodiment provided herein may apply to any other embodiment provided herein. Furthermore, any compound and pharmaceutically acceptable salts thereof described herein or composition described herein may be used in any method provided herein, and any method provided herein may be used to produce or to utilize any compound and pharmaceutically acceptable salts thereof described herein or composition described herein.

Throughout this application, the term “about” is used to indicate that a value includes the standard deviation of error for the device or method being employed to determine the value.

### COMPOUNDS

Provided herein are compounds or an atropisomer, stereoisomer, tautomer, or pharmaceutically acceptable salt thereof of formula (I):

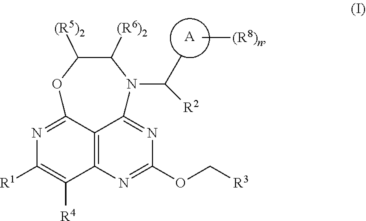

wherein;
Ring A is R⁸-substituted or unsubstituted 3-10 membered heterocycle, or R⁸-substituted or unsubstituted 5-10 membered heteroaryl;
R⁸ is independently halogen, CN, NH₂, NHC₁₋₃alkyl, R^{8A}-substituted or unsubstituted C₁₋₆alkyl, or R^{8A}-substituted or unsubstituted C₁₋₆haloalkyl; R^{8A} is halogen, unsubstituted C₁₋₃alkyl, or unsubstituted C₁₋₃haloalkyl;
n is 1 or 2;
R¹ is R⁷-substituted or unsubstituted indolyl, R⁷-substituted or unsubstituted benzofuranyl, R⁷-substituted or unsubstituted naphthyl, R⁷-substituted or unsubstituted indazolyl, R⁷-substituted or unsubstituted indenyl, R⁷-substituted or unsubstituted benzothiazolyl, R⁷-substituted or unsubstituted isoquinolinyl, R^{7A}-substituted or unsubstituted phenyl, or R^{7A}-substituted or unsubstituted pyridinyl;
each R⁷ is independently hydrogen, halogen, CN, CH₂OH, —OH, NH₂, N(Me)₂, unsubstituted C₁₋₃alkyl, unsubstituted C₂₋₅ alkynyl, unsubstituted C₁₋₃ haloalkyl, or unsubstituted cyclopropyl;
each R^{7a} is independently hydrogen, halogen, NH₂, N(Me)₂, unsubstituted C₁₋₃ alkyl, unsubstituted C₁₋₃haloalkyl, or unsubstituted cyclopropyl;
R² is OH, unsubstituted C₁₋₃alkoxy, unsubstituted C₁₋₃alkyl, or unsubstituted C₁₋₃haloalkyl;
R³ is R⁹-substituted or unsubstituted 4-10 membered heterocycle comprising N, S, or O;
R⁹ is independently halogen, oxo, unsubstituted C₁₋₃ alkyl, unsubstituted C₁₋₃ haloalkyl, unsubstituted C₁₋₃ alkoxy, R¹⁰-substituted or unsubstituted C₁₋₃ alkylidene, or R¹⁰-substituted or unsubstituted C₃₋₄ cycloalkyl, or R¹⁰-substituted or unsubstituted 3 or 4-membered heterocycle;
or wherein two R⁹ together form a C₃₋₅ cycloalkyl or 3-5 membered heterocycle;
R¹⁰ is hydrogen or halogen;
R⁴ is hydrogen, halogen, unsubstituted C₁₋₃alkyl, unsubstituted C₁₋₃alkoxy, or unsubstituted C₁₋₃haloalkyl; and
R⁵ and R⁶ are each independently hydrogen or unsubstituted C₁₋₃alkyl.

In one embodiment of the compounds or a stereoisomer, atropisomer, tautomer, or pharmaceutically acceptable salt thereof described herein, R⁵ and R⁶ are each hydrogen. In another embodiment, R⁵ is CH₃ and R⁶ is hydrogen. In another embodiment, R⁶ is hydrogen or unsubstituted C₁₋₃alkyl. In another embodiment, R⁶ is CH₃ and R⁵ is hydrogen.

In one embodiment, the compound or an atropisomer, stereoisomer, tautomer, or pharmaceutically acceptable salt thereof of formula (I) comprises formula (Ia):

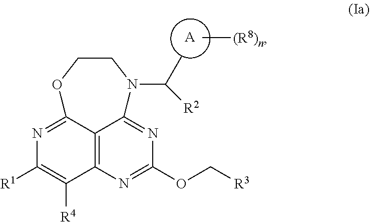

wherein R¹, R², R³, R⁴, R⁸, Ring A and n are as described herein.

In one embodiment of the compounds or a stereoisomer, atropisomer, tautomer, or pharmaceutically acceptable salt thereof described herein, R¹ is R⁷-substituted or unsubstituted indolyl, R⁷-substituted or unsubstituted benzofuranyl, R⁷-substituted or unsubstituted naphthyl, R⁷-substituted or unsubstituted indazolyl, R⁷-substituted or unsubstituted indenyl, R⁷-substituted or unsubstituted benzothiazolyl, R⁷-substituted or unsubstituted isoquinolinyl, R^{7A}-substituted or unsubstituted phenyl, or R^{7A}-substituted or unsubstituted pyridinyl.

In another embodiment, R¹ is R⁷-substituted or unsubstituted indolyl, R⁷-substituted or unsubstituted benzofuranyl, R⁷-substituted or unsubstituted naphthyl, R⁷-substituted or unsubstituted indazolyl, R⁷-substituted or unsubstituted indenyl, R⁷-substituted or unsubstituted benzothiazolyl, or R⁷-substituted or unsubstituted isoquinolinyl. In another embodiment, R¹ is R⁷-substituted or unsubstituted naphthyl or R⁷-substituted or unsubstituted isoquinolinyl.

In one embodiment, R¹ is R⁷-substituted or unsubstituted naphthyl or R⁷-substituted or unsubstituted isoquinolinyl. In another embodiment, R¹ is R^{7A}-substituted or unsubstituted phenyl, or R^{7A}-substituted or unsubstituted pyridinyl. In another embodiment, R¹ is

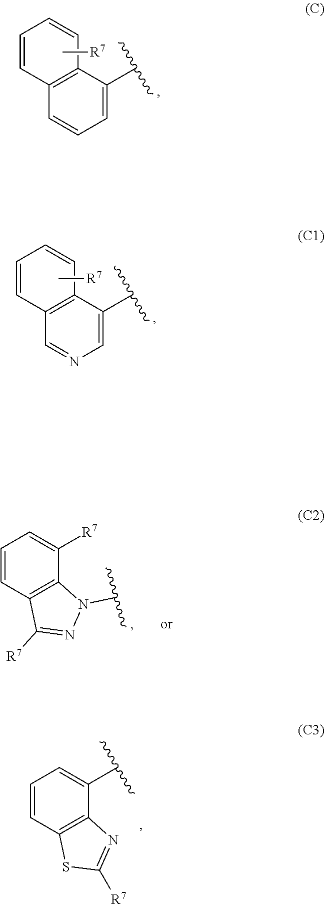

where R⁷ is as defined herein.

In another embodiment, R¹ is

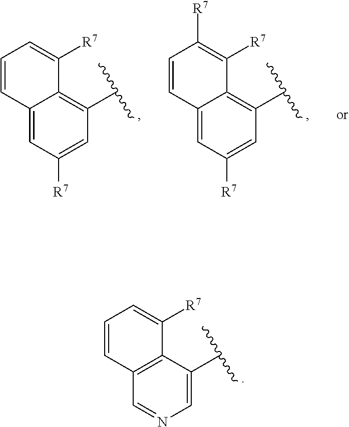

In another embodiment, R¹ is

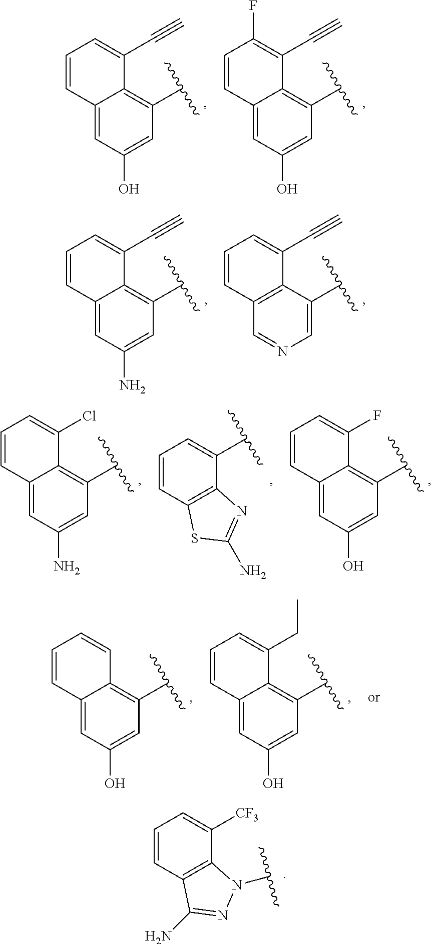

In some embodiments, each R⁷ is independently hydrogen, halogen, CN, CH₂OH, —OH, NH₂, N(Me)₂, unsubstituted C₁₋₃alkyl, or unsubstituted C₂₋₅ alkynyl. In one embodiment, each R⁷ is independently hydrogen, halogen, —OH, NH₂, N(Me)₂, unsubstituted C₁₋₃alkyl, or unsubstituted C₂₋₅ alkynyl. In another embodiment, each R⁷ is independently hydrogen, halogen, —OH, NH₂, methyl, ethyl, or unsubstituted C₂₋₃ alkynyl. In another embodiment, each R⁷ is independently halogen or —OH. In another embodiment, each R⁷ is independently halogen, —OH, NH₂, or unsubstituted C₂₋₃ alkynyl.

In one embodiment, R¹ is R^{7A}-substituted or unsubstituted phenyl, or R^{7A}-substituted or unsubstituted pyridinyl. In one such embodiment, R¹ is R^{7A}-substituted or unsubstituted phenyl. In another such embodiment, R¹ is R^{7A}-substituted or unsubstituted pyridinyl.

In one embodiment, R¹ is

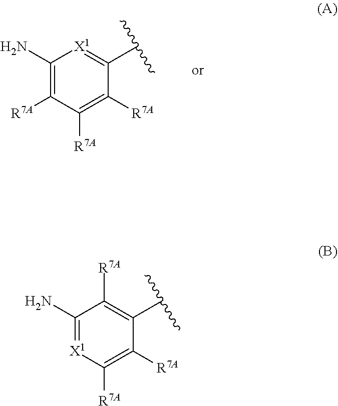

wherein X¹ is N, CH, CF, or CCl; and R^{7A} is hydrogen, halogen, unsubstituted C₁₋₃ alkyl, or unsubstituted C₁₋₃ haloalkyl.

In one embodiment, where R¹ is a moiety of formula (A), X¹ is N and R^{7A} is as described herein. In another embodiment, where R¹ is a moiety of formula (B), X¹ is N and R^{7A} is as described herein. In still another embodiment, where R¹ is a moiety of formula (A), X¹ is CH, CF, or CCl, where R^{7A} is a described herein.

In some embodiments, R¹ is

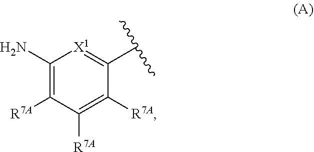

wherein,
X¹ is N, CH, CF, or CCl; and
R^{7A} is hydrogen, halogen, unsubstituted C₁₋₃ alkyl, or unsubstituted C₁₋₃ haloalkyl.

In one such embodiment, the moiety of formula (A) comprises a moiety where R¹ is

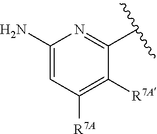

wherein R^{7A} is hydrogen, halogen, unsubstituted C₁₋₃ alkyl or unsubstituted C₁₋₃ haloalkyl.

In one embodiment, R¹ is

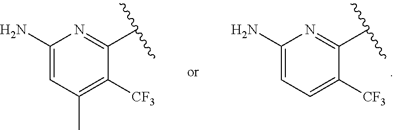 [SMILES: *c1nc(N)cc(C)c1C(F)(F)F ; *c1nc(N)ccc1C(F)(F)F]

In some embodiments, R¹ is

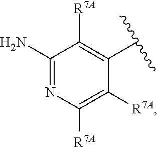

wherein R^{7A} is hydrogen, halogen, unsubstituted G1-3 alkyl, or unsubstituted G1-3 haloalkyl.

In some embodiments, R¹ is

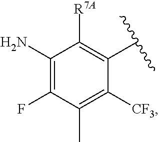

wherein R^{7A} is hydrogen, F, or C₁. In one such embodiment, R^{7A} is F. In another such embodiment, R^{7A} is C₁. In one embodiment, R^{7A} is methyl. In one embodiment, R^{7A} is hydrogen.

Still further provided herein, in some embodiments, the moiety of R¹ is

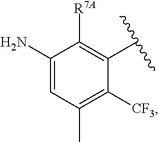

wherein R^{7A} is hydrogen, F, or C₁. In one such embodiment, R^{7A} is F. In another such embodiment, R^{7A} is C₁. In one embodiment, R^{7A} is methyl. In one embodiment, R^{7A} is hydrogen.

In one embodiment of the compounds or a stereoisomer, atropisomer, tautomer, or pharmaceutically acceptable salt thereof described herein, R¹ is

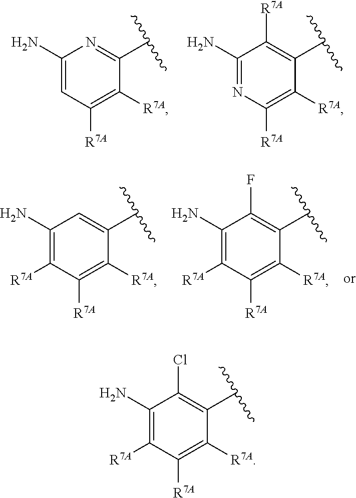

In another embodiment of the compounds or a stereoisomer, atropisomer, tautomer, or pharmaceutically acceptable salt thereof described herein, R¹ is

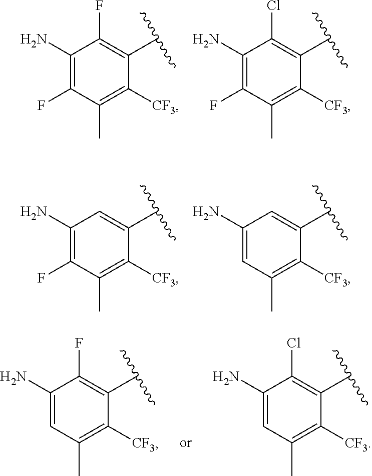

In still another embodiment of the compounds or a stereoisomer, atropisomer, tautomer, or pharmaceutically acceptable salt thereof described herein, R¹ is

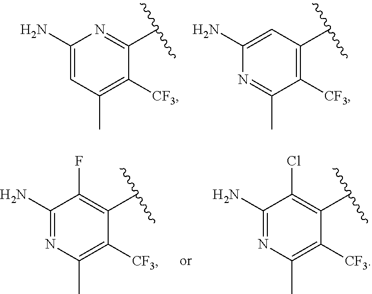

In one embodiment, each R^{7a} is independently hydrogen, halogen, NH₂, unsubstituted C₁₋₃ alkyl, or unsubstituted C₁₋₃haloalkyl. In another embodiment, each R^{7a} is independently hydrogen, F, C₁, NH₂, methyl, or CF₃.

In one embodiment of the compounds or a stereoisomer, atropisomer, tautomer, or pharmaceutically acceptable salt thereof described herein, R⁴ is unsubstituted C₁₋₃alkyl, unsubstituted C₁₋₃alkoxy, or unsubstituted C₁₋₃haloalkyl. In one such embodiment, R⁴ is OCH₃. In another embodiment, R⁴ is Cl or F. In one such embodiment, R⁴ is F. In another such embodiment, R⁴ is Cl. In one embodiment, R¹ is a moiety of formula (A) as described herein and R⁴ is Cl or F. In one such embodiment, R¹ is a moiety of formula (A) as described herein, wherein X¹ is N or X¹ is CH, CF, or CCl, and R⁴ is F. In another such embodiment, R¹ is a moiety of formula (A) as described herein, wherein X¹ is N or X¹ is CH, CF, or CCl, and R⁴ is Cl. In such embodiments, R^{7A} is hydrogen, halogen, methyl, and/or CF₃ as described herein.

In another embodiment, R¹ is a moiety of formula (C), (C1), (C2), or (C3) as described herein wherein R⁴ is Cl or F. In one such embodiment, R⁴ is F. In another such embodiment, R⁴ is Cl.

In one embodiment of the compounds or a stereoisomer, atropisomer, tautomer, or pharmaceutically acceptable salt thereof described herein, R³ is R⁹-substituted or unsubstituted 4-10 membered heterocycle comprising one or more N or O ring heteroatoms. In one such embodiment, R³ is R⁹-substituted or unsubstituted 4-10 membered heterocycle comprising one or more N heteroatoms. In one such embodiment, the heterocycle comprises 1 or 2 ring N heteroatoms.

In one embodiment, R³ is

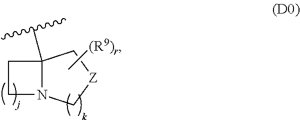

wherein
Z is C(R⁹)₂ or O;
R⁹ is hydrogen, halogen or R¹⁰-substituted or unsubstituted C₁₋₃ alkylidene;
or wherein two R⁹ together form a C₃₋₅ cycloalkyl or 3-5 membered heterocycle
r is an integer of 0-12;
j is 1, 2, or 3; and
k is 1 or 2.

In one embodiment, Z is O and k is 2. In another embodiment, Z is O, k is 2, and j is 2. In another embodiment, Z is C(R⁹)₂, where R⁹ is as described herein, k is 1, and j is 2. In one embodiment, R³ is

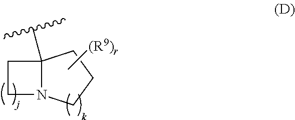

wherein
R⁹ is halogen, —OCF₃, —OCHF₂, —OCH₂F, R¹⁰-substituted or unsubstituted C₁₋₃ alkylidene, or two R⁹ together form a R¹⁰-substituted or unsubstituted C₃₋₅ cycloalkyl;
r is an integer of 0-12;
j is 1, 2, or 3; and
k is 1 or 2.

In one embodiment, where R³ is a moiety of formula (D), R⁹ is halogen, R¹⁰-substituted or unsubstituted C₁₋₃ alkylidene, or two R⁹ together form a R¹⁰-substituted or unsubstituted C₃₋₅ cycloalkyl. In one such embodiment, two R⁹ form a R¹⁰-substituted or unsubstituted cyclopropyl. In one such embodiment, R¹⁰ is halogen (e.g. F). In another embodiment, where R³ is a moiety of formula (D), k is 1, j is 2, and R⁹ is a described herein and r is 0, 1, 2, 3, or 4. In one embodiment, R⁹ is halogen or R¹⁰-substituted or unsubstituted C₁₋₃ alkylidene and r is 2 or 3. In one embodiment, R³ is

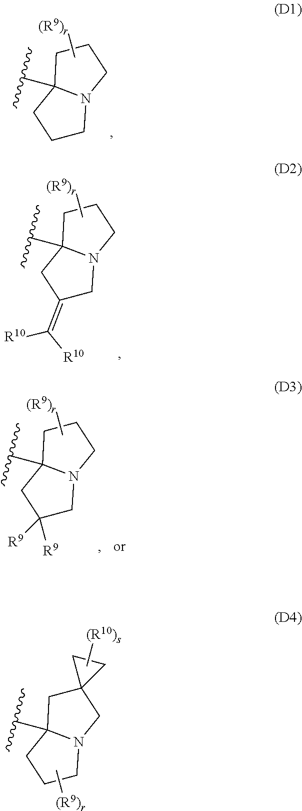

each R⁹ is independently halogen or R¹⁰-substituted or unsubstituted C₁₋₃ alkylidene;
each R¹⁰ is independently hydrogen or halogen;
r is 0, 1, or 2; and
s is 0, 1, or 2

In one embodiment, R³ is a moiety of (D1), (D2), or (D3), wherein
each R⁹ is independently halogen or R¹⁰-substituted or unsubstituted C₁₋₃ alkylidene;
each R¹⁰ is independently hydrogen or halogen;
r is 1 or 2; and
s is 0, 1, or 2.

In another embodiment, R³ is (D1), where r is 1. In another embodiment, R³ is (D2) where r is 0 and each R¹⁰ is independently hydrogen or F. In another embodiment, R³ is (D3) where r is 0 and each R⁹ is independently hydrogen or halogen. In another embodiment, R³ is (D4), wherein R¹⁰ is halogen and s is 0, 1, or 2.

In one embodiment, R³ is

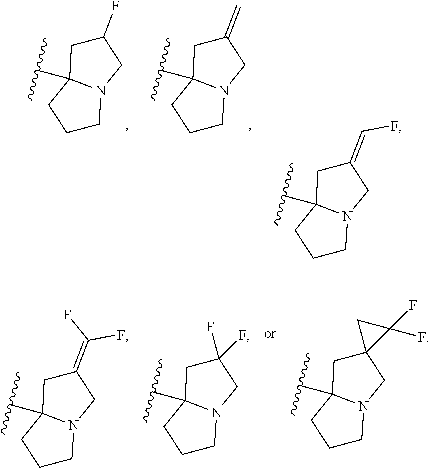

In one embodiment, R³ is

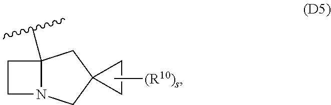

wherein R¹⁰ is halogen and s is 0, 1, or 2. In one such embodiment, R¹⁰ is F and s is 1 or 2.

In one embodiment R³ is (E)

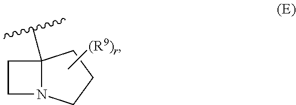

wherein, each R⁹ is independently halogen, oxo, or unsubstituted C₁₋₃ alkyl; and
r is 0, 1, or 2. In one such embodiment, R⁹ is F and r is 1 or 2.

In one embodiment, R³ is

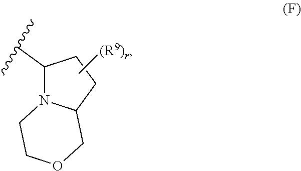

wherein, each R⁹ is independently halogen, oxo, or unsubstituted C₁₋₃ alkyl; and
r is 0, 1, or 2. In one embodiment, R⁹ is halogen and r is 1. In another embodiment, r is 0. In one embodiment, the moiety of formula (F) comprises a compound of formula

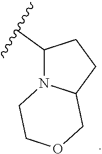 [SMILES: *C1CCC2COCCN12]

In a further embodiment, R³ is

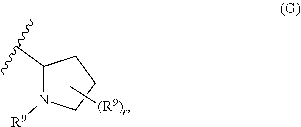

wherein,
each R⁹ is independently halogen, oxo, or unsubstituted C₁₋₃ alkyl;
or wherein two R⁹ together form a C₃₋₅ cycloalkyl or 3-5 membered heterocycle; and r is 1 or 2.

In one embodiment, R³ is

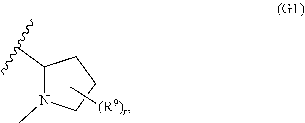

wherein,
each R⁹ is independently halogen, oxo, or unsubstituted C₁₋₃ alkyl;
or wherein two R⁹ together form a C₃₋₅ cycloalkyl or 3-5 membered heterocycle; and
r is 1 or 2. In one such embodiment, R⁹ is halogen and r is 1 or 2. In one such embodiment, R⁹ is halogen and r is 1.

In one embodiment, R³ is

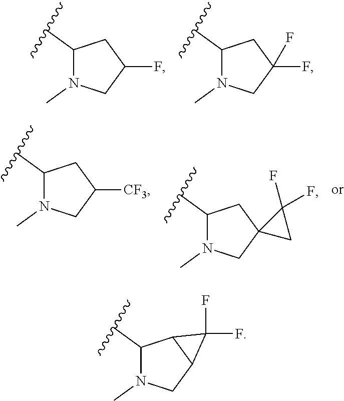

In one embodiment of the compounds or a stereoisomer, atropisomer, tautomer, or pharmaceutically acceptable salt thereof described herein, R² is OH or unsubstituted C₁₋₃alkoxy. In one such embodiment, R² is OH. In another embodiment, R² is unsubstituted C₁₋₃alkoxy or unsubstituted C₁₋₃alkyl. In another embodiment, R² is unsubstituted C₁₋₃alkyl or unsubstituted C₁₋₃haloalkyl. In one such embodiment, R² is CHF₂, CH₂F, or CF₃. In another embodiment, R² is unsubstituted C₁₋₃alkyl. In one such embodiment, R² is methyl.

In one embodiment of the compounds or a stereoisomer, atropisomer, tautomer, or pharmaceutically acceptable salt thereof described herein, Ring A is R⁸-substituted or unsubstituted 3-10 membered heterocycle, or R⁸-substituted or unsubstituted 5-10 membered heteroaryl as described herein. In one embodiment, Ring A is R⁸-substituted or unsubstituted 3-6 membered heterocycle comprising at least 1 heterocyclic ring nitrogen atom, or R⁸-substituted or unsubstituted 5-6 membered heteroaryl comprising at least 1 heterocyclic ring nitrogen atom. In one such embodiment, the heterocyclic ring or heteroaryl comprises 1 or 2 ring nitorgen atoms. In another embodiment, Ring A is R⁸-substituted or unsubstituted 5 or 6 membered heterocycle comprising at least 1 heterocyclic ring nitrogen atom. In another embodiment, Ring A is R⁸-substituted or unsubstituted 6 membered heteroaryl comprising at least 1 heterocyclic ring nitrogen atom. In one embodiment, Ring A is R⁸-substituted or unsubstituted pyridinyl, R⁸-substituted or unsubstituted pyrimidinyl or R⁸-substituted or unsubstituted pyrazinyl.

In another embodiment, Ring A is a moiety of formula,

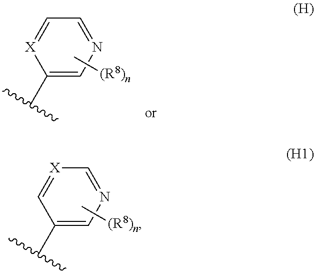

wherein X is CH or N and n is 1 or 2. In one such embodiment, where Ring A is a moiety of formula (H), X is N and n is 1.

In one embodiment, where Ring A is a moiety of formula (H) or (H1), R⁸ is independently halogen, CN, NH₂, or R^{8A}-substituted or unsubstituted C₁₋₆alkyl. In another embodiment, R⁸ is independently halogen, CN, or NH₂. In another embodiment, R⁸ is independently R^{8A}-substituted or unsubstituted C₁₋₆alkyl, or R^{8A}-substituted or unsubstituted C₁₋₆haloalkyl. In another embodiment, R⁸ is NH₂. In another embodiment, R⁸ is NH₂ and n is 1.

In another embodiment, Ring A is a moiety of formula;

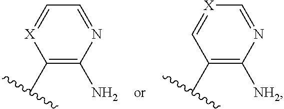

wherein X is CH or N. In one embodiment, X is CH. In one embodiment, X is N.

In some embodiments, Ring A is a moiety of formula:

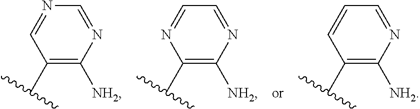 [SMILES: *c1cncnc1N ; *c1nccnc1N ; *c1cccnc1N]

In one embodiment of the compounds or a stereoisomer, atropisomer, tautomer, or pharmaceutically acceptable salt thereof described herein, Ring A is a moiety of formula

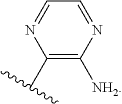 [SMILES: *c1nccnc1N]

In one embodiment of the compound or a stereoisomer, atropisomer, tautomer, or pharmaceutically acceptable salt thereof of formula (I) or (Ia) described herein, the compound comprises a structure formula (II):

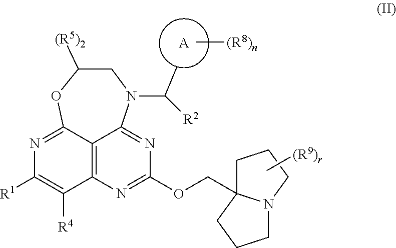

including a stereoisomer, atropisomer, tautomer, or pharmaceutically acceptable salt thereof, wherein R¹, R², R⁴, R⁵, R⁸, R⁹, n, r, and Ring A are as described herein.

In one embodiment of the compounds or a stereoisomer, atropisomer, tautomer, or pharmaceutically acceptable salt thereof of formula (II) described herein, the compound of formula (II) comprises a structure of:

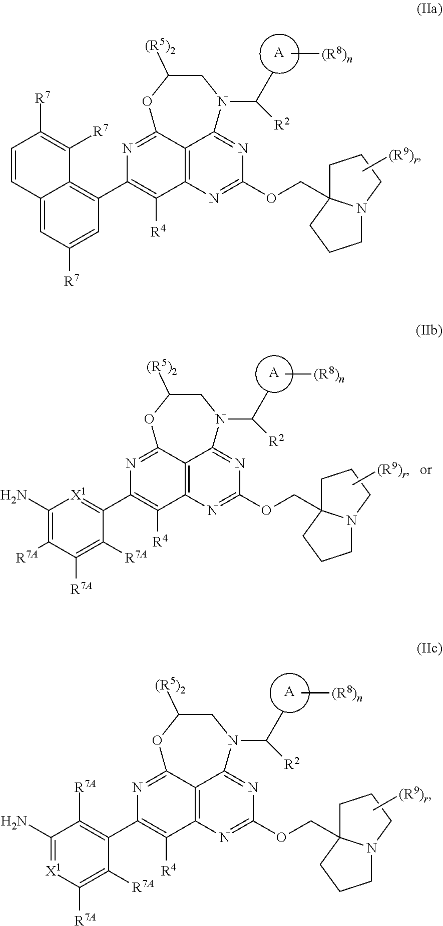

including a stereoisomer, atropisomer, tautomer, or pharmaceutically acceptable salt thereof.

In one embodiment of the compounds or a stereoisomer, atropisomer, tautomer, or pharmaceutically acceptable salt thereof of formula (II), (IIIa), (IIb), or (IIc) described herein, R⁹ is independently halogen or R¹⁰-substituted or unsubstituted C₁₋₃ alkylidene; each R¹⁰ is independently hydrogen or halogen; and r is 1 or 2. In another such embodiment, two R⁹ together form a C₃₋₅ cycloalkyl or 3-5 membered heterocycle. In one such embodiment, two R⁹ together form a R¹⁰-substituted or unsubstituted cyclopropyl. In one such embodiment, R¹⁰ is hydrogen or F. In one embodiment, the compound comprises the moiety:

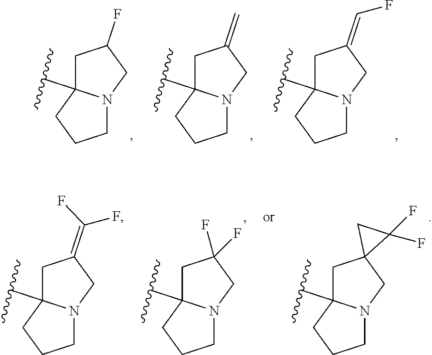

Further provided herein are compounds or a stereoisomer, atropisomer, tautomer, or pharmaceutically acceptable salt thereof of formula (III):

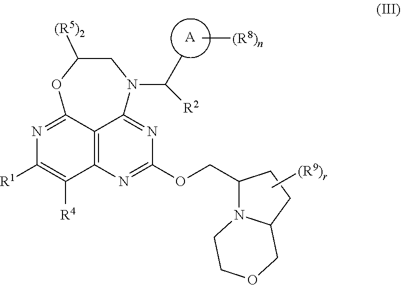

including a stereoisomer, atropisomer, tautomer, or pharmaceutically acceptable salt thereof, wherein R¹, R², R⁴, R⁵, R⁸, R⁹, n, r, and Ring A are as described herein.

In one embodiment of the compounds or a stereoisomer, atropisomer, tautomer, or pharmaceutically acceptable salt thereof of formula (III) described herein, the compound of formula (III) comprises a structure of:

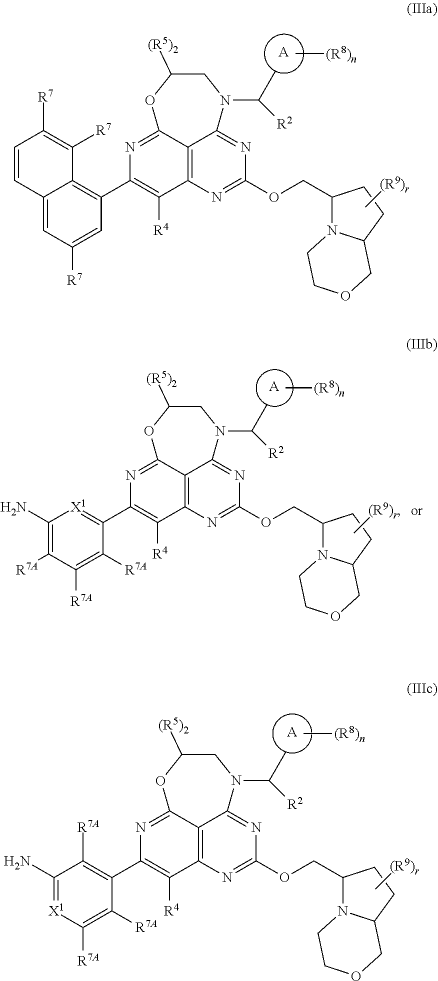

including a stereoisomer, atropisomer, tautomer, or pharmaceutically acceptable salt thereof. In one embodiment of the compounds or a stereoisomer, atropisomer, tautomer, or pharmaceutically acceptable salt thereof of formula (III), (IIIa), (IIIb), or (IIIc) described herein, comprises the moiety:

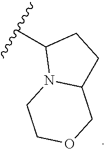 [SMILES: *C1CCC2COCCN12]

Further provided herein are compounds or a stereoisomer, atropisomer, tautomer, or pharmaceutically acceptable salt thereof of formula (IV):

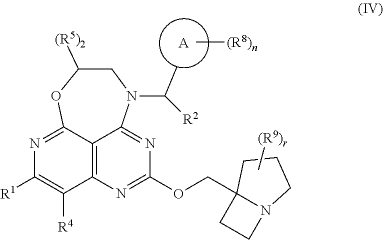

including a stereoisomer, atropisomer, tautomer, or pharmaceutically acceptable salt thereof, wherein R¹, R², R⁴, R⁵, R⁸, R⁹, n, r, and Ring A are as described herein.

In one embodiment of the compounds or a stereoisomer, atropisomer, tautomer, or pharmaceutically acceptable salt thereof of formula (IV) described herein, the compound of formula (IV) comprises a structure of:

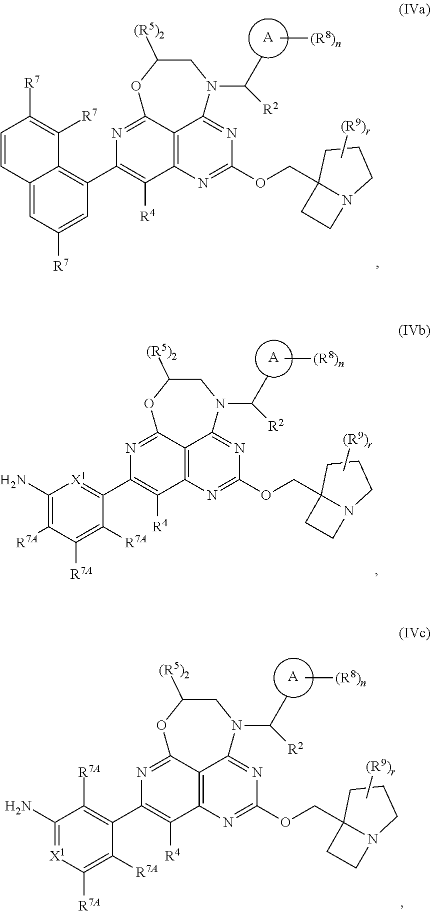

including a stereoisomer, atropisomer, tautomer, or pharmaceutically acceptable salt thereof. In one embodiment of the compounds or a stereoisomer, atropisomer, tautomer, or pharmaceutically acceptable salt thereof of formula (IV), (IVa), (IVb), or (IVc) described herein, comprises the moiety:

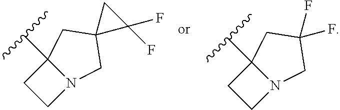 [SMILES: *C12CCN1CC1(C2)CC1(F)F ; *C12CCN1CC(F)(F)C2]

Further provided herein are compounds or a stereoisomer, atropisomer, tautomer, or pharmaceutically acceptable salt thereof of formula (V):

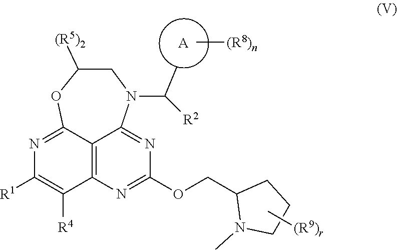

including a stereoisomer, atropisomer, tautomer, or pharmaceutically acceptable salt thereof, wherein R¹, R², R⁴, R⁵, R⁸, R⁹, n, r, and Ring A are as described herein.

In one embodiment of the compounds or a stereoisomer, atropisomer, tautomer, or pharmaceutically acceptable salt thereof of formula (IV) described herein, the compound of formula (IV) comprises a structure of:

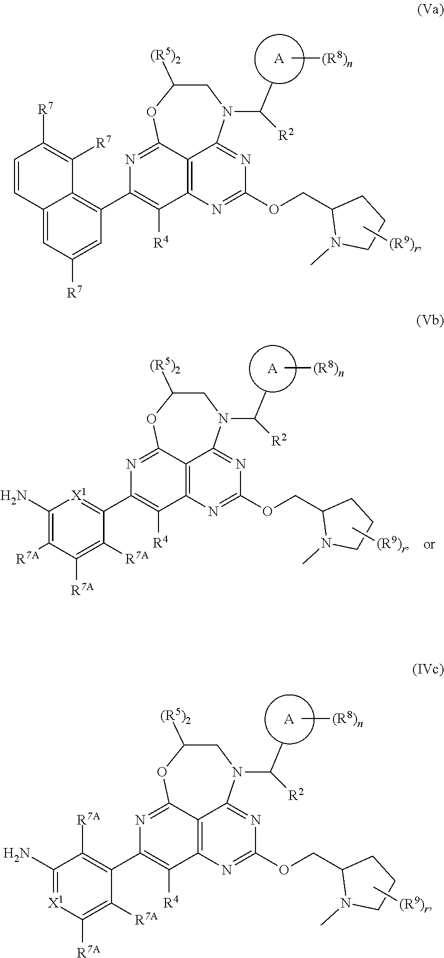

including a stereoisomer, atropisomer, tautomer, or pharmaceutically acceptable salt thereof. In one embodiment of the compounds or a stereoisomer, atropisomer, tautomer, or pharmaceutically acceptable salt thereof of formula (V), (Va), (Vb), or (Vc) described herein, comprises the moiety:

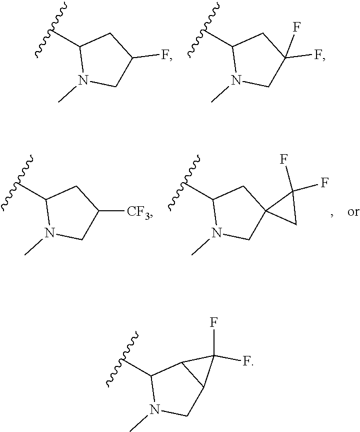

In one embodiment of the compounds or a stereoisomer, atropisomer, tautomer, or pharmaceutically acceptable salt thereof of formula (II), (IIa), (IIb), (IIc), (III), (IIIa), (IIIb), (IIIc), (IV), (IVa), (IVb), (IVc), (V), (Va), (Vb), or (Vc) described herein, Ring A comprises a moiety of formula:

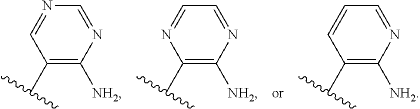 [SMILES: *c1cncnc1N ; *c1nccnc1N ; *c1cccnc1N]

In one embodiment of the compounds or a stereoisomer, atropisomer, tautomer, or pharmaceutically acceptable salt thereof of formula (II), (IIb), (IIc), (III), (IIIb), (IIIc), (IV), (IVb), (IVc), (V), (Vb), or (Vc) described herein, X¹ is N and each R^{7a} is independently hydrogen, halogen, NH₂, unsubstituted C₁₋₃ alkyl, or unsubstituted C₁₋₃haloalkyl. In another such embodiment, where the compound is of formula (IIb) as described herein X¹ is CH, CF, or CCl and each R^{7a} is independently hydrogen, F, C₁, NH₂, methyl, or CF₃. In one embodiment of the compounds or a stereoisomer, atropisomer, tautomer, or pharmaceutically acceptable salt thereof of formula (II), (IIb), or (IIc) described herein, the compound comprises the moiety:

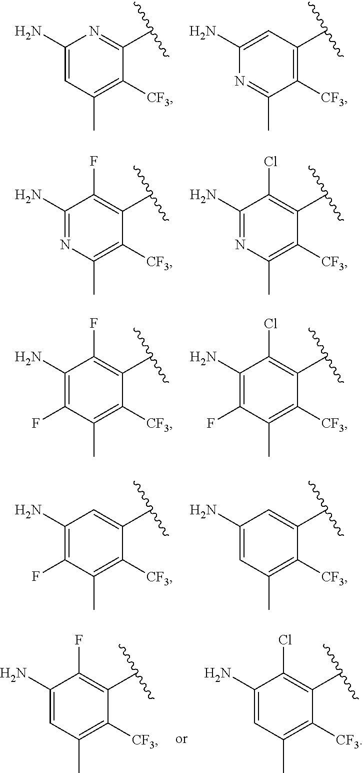

In one embodiment of the compounds or a stereoisomer, atropisomer, tautomer, or pharmaceutically acceptable salt thereof of formula (I), (la), (II), (IIa), (IIb), (IIc), (III), (IIIa), (IIIb), (IIIc), (IV), (IVa), (IVb), (IVc), (V), (Va), (Vb), or (Vc) described herein, the compound is one set forth in Table 1 below.

TABLE 1
Representative Compounds

| Ex. | | |
|---|---|---|
| No. | Structure | Name |
| 1 | [symbol] | 4-(10-((R)-1-(2-aminopyridin-3-yl)ethyl)- |
| | | 2-((3,3-difluoro-1- |
| | | azabicyclo[3.2.0]heptan-5-yl)methoxy)-4- |
| | | fluoro-9,10-dihydro-8H-7-oxa-1,3,6,10- |
| | | tetraazacyclohepta[de]naphthalen-5-yl)- |
| | | 5-ethynylnaphthalen-2-ol |
| 2 | 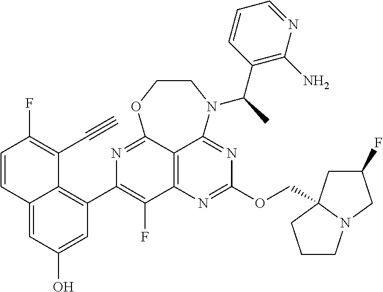 [SMILES: C#Cc1c(F)ccc2cc(O)cc(-c3nc4c5c(nc(OC[C@@]67CCCN6C[C@H](F)C7)nc5c3F)N([C@H](C)c3cccnc3N)CCO4)c12] | 4-(10-((R)-1-(2-aminopyridin-3-yl)ethyl)- 4-fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5-yl)- 5-ethynyl-6-fluoronaphthalen-2-ol |
| 3 | 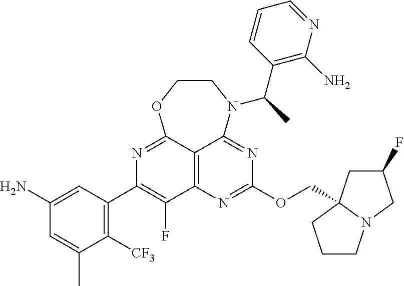 [SMILES: Cc1cc(N)cc(-c2nc3c4c(nc(OC[C@@]56CCCN5C[C@H](F)C6)nc4c2F)N([C@H](C)c2cccnc2N)CCO3)c1C(F)(F)F] | 3-((R)-1-(5-(5-amino-3-methyl-2- (trifluoromethyl)phenyl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 4 | 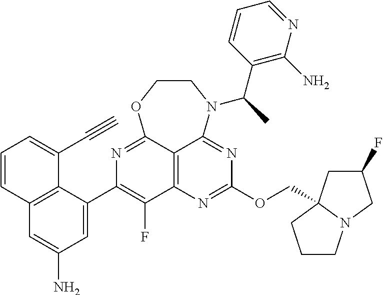 [SMILES: C#Cc1cccc2cc(N)cc(-c3nc4c5c(nc(OC[C@@]67CCCN6C[C@H](F)C7)nc5c3F)N([C@H](C)c3cccnc3N)CCO4)c12] | 3-((R)-1-(5-(3-amino-8- ethynylnaphthalen-1-yl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 5 | 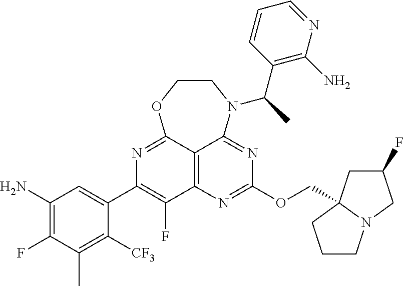 [SMILES: Cc1c(F)c(N)cc(-c2nc3c4c(nc(OC[C@@]56CCCN5C[C@H](F)C6)nc4c2F)N([C@H](C)c2cccnc2N)CCO3)c1C(F)(F)F] | 3-((R)-1-(5-(5-amino-4-fluoro-3-methyl-2- (trifluoromethyl)phenyl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 6 | 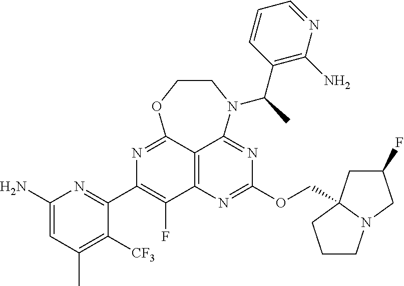 [SMILES: Cc1cc(N)nc(-c2nc3c4c(nc(OC[C@@]56CCCN5C[C@H](F)C6)nc4c2F)N([C@H](C)c2cccnc2N)CCO3)c1C(F)(F)F] | 6-(10-((R)-1-(2-aminopyridin-3-yl)ethyl)- 4-fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5-yl)- 4-methyl-5-(trifluoromethyl)pyridin-2- amine |
| 7 | 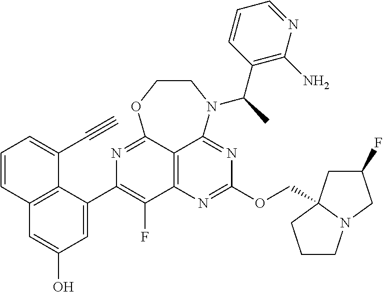 [SMILES: C#Cc1cccc2cc(O)cc(-c3nc4c5c(nc(OC[C@@]67CCCN6C[C@H](F)C7)nc5c3F)N([C@H](C)c3cccnc3N)CCO4)c12] | 4-(10-((R)-1-(2-aminopyridin-3-yl)ethyl)- 4-fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5-yl)- 5-ethynylnaphthalen-2-ol |
| 8 | 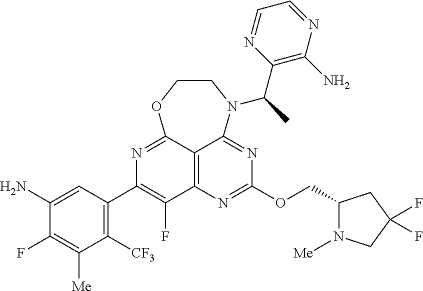 [SMILES: Cc1c(F)c(N)cc(-c2nc3c4c(nc(OC[C@@H]5CC(F)(F)CN5C)nc4c2F)N([C@H](C)c2nccnc2N)CCO3)c1C(F)(F)F] | 3-((R)-1-(5-(5-amino-4-fluoro-3-methyl-2- (trifluoromethyl)phenyl)-2-(((S)-4,4- difluoro-1-methylpyrrolidin-2-yl)methoxy)- 4-fluoro-8,9-dihydro-10H-7-oxa-1,3,6,10- tetraazacyclohepta[de]naphthalen-10- yl)ethyl)pyrazin-2-amine |
| 9 | 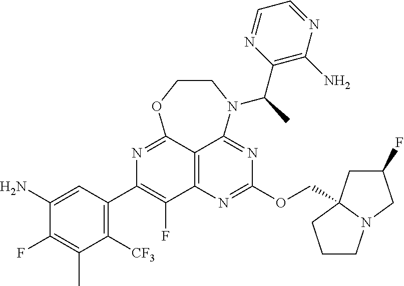 [SMILES: Cc1c(F)c(N)cc(-c2nc3c4c(nc(OC[C@@]56CCCN5C[C@H](F)C6)nc4c2F)N([C@H](C)c2nccnc2N)CCO3)c1C(F)(F)F] | 3-((R)-1-(5-(5-amino-4-fluoro-3-methyl-2- (trifluoromethyl)phenyl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyrazin-2-amine |
| 10 | 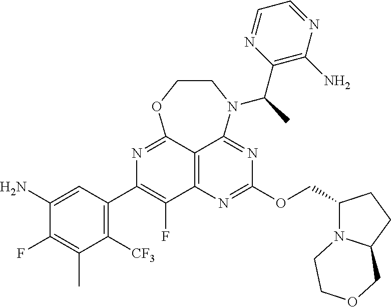 [SMILES: Cc1c(F)c(N)cc(-c2nc3c4c(nc(OC[C@@H]5CC[C@H]6COCCN65)nc4c2F)N([C@H](C)c2nccnc2N)CCO3)c1C(F)(F)F] | 3-((R)-1-(5-(5-amino-4-fluoro-3-methyl-2- (trifluoromethyl)phenyl)-4-fluoro-2- (((6S,8aS)-hexahydro-1H-pyrrolo[2,1- c][1,4]oxazin-6-yl)methoxy)-8,9-dihydro- 10H-7-oxa-1,3,6,10- tetraazacyclohepta[de]naphthalen-10- yl)ethyl)pyrazin-2-amine |
| 11 | 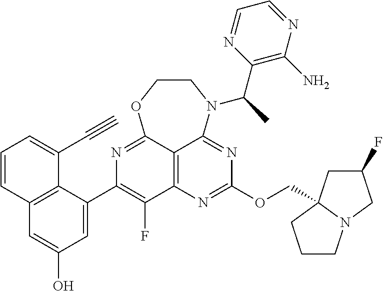 [SMILES: C#Cc1cccc2cc(O)cc(-c3nc4c5c(nc(OC[C@@]67CCCN6C[C@H](F)C7)nc5c3F)N([C@H](C)c3nccnc3N)CCO4)c12] | 4-(10-((R)-1-(3-aminopyrazin-2-yl)ethyl)- 4-fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5-yl)- 5-ethynylnaphthalen-2-ol |
| 12 | 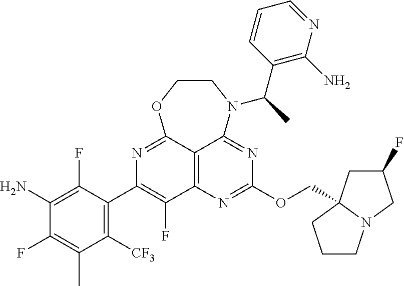 [SMILES: Cc1c(F)c(N)c(F)c(-c2nc3c4c(nc(OC[C@@]56CCCN5C[C@H](F)C6)nc4c2F)N([C@H](C)c2cccnc2N)CCO3)c1C(F)(F)F] | 3-((1R)-1-(5-(3-amino-2,4-difluoro-5- methyl-6-(trifluoromethyl)phenyl)-4- fluoro-2-(((2R,7aS)-2-fluorotetrahydro- 1H-pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 13 | 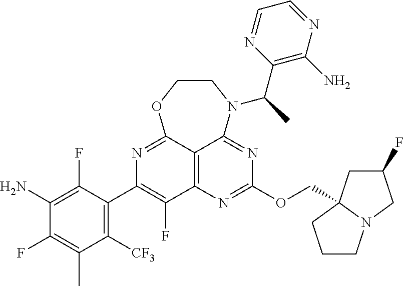 [SMILES: Cc1c(F)c(N)c(F)c(-c2nc3c4c(nc(OC[C@@]56CCCN5C[C@H](F)C6)nc4c2F)N([C@H](C)c2nccnc2N)CCO3)c1C(F)(F)F] | 3-((1R)-1-(5-(3-amino-2,4-difluoro-5- methyl-6-(trifluoromethyl)phenyl)-4- fluoro-2-(((2R,7aS)-2-fluorotetrahydro- 1H-pyrrolizin-7a(5H)-yl)methoxy)-8,9- dihydro-10H-7-oxa-1,3,6,10- tetraazacyclohepta[de]naphthalen-10- yl)ethyl)pyrazin-2-amine |
| 14 | 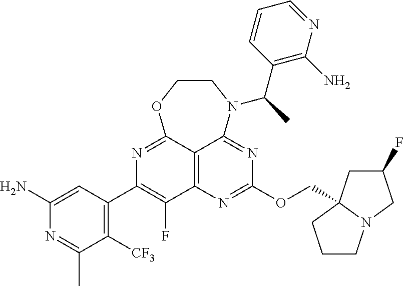 [SMILES: Cc1nc(N)cc(-c2nc3c4c(nc(OC[C@@]56CCCN5C[C@H](F)C6)nc4c2F)N([C@H](C)c2cccnc2N)CCO3)c1C(F)(F)F] | 4-(10-((R)-1-(2-aminopyridin-3-yl)ethyl)- 4-fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5-yl)- 6-methyl-5-(trifluoromethyl)pyridin-2- amine |
| 15 | 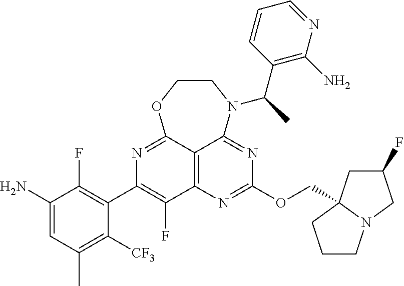 [SMILES: Cc1cc(N)c(F)c(-c2nc3c4c(nc(OC[C@@]56CCCN5C[C@H](F)C6)nc4c2F)N([C@H](C)c2cccnc2N)CCO3)c1C(F)(F)F] | 3-((1R)-1-(5-(3-amino-2-fluoro-5-methyl- 6-(trifluoromethyl)phenyl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 16 | 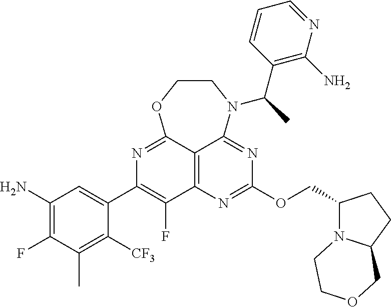 [SMILES: Cc1c(F)c(N)cc(-c2nc3c4c(nc(OC[C@@H]5CC[C@H]6COCCN65)nc4c2F)N([C@H](C)c2cccnc2N)CCO3)c1C(F)(F)F] | 3-((R)-1-(5-(5-amino-4-fluoro-3-methyl-2- (trifluoromethyl)phenyl)-4-fluoro-2- (((6S,8aS)-hexahydro-1H-pyrrolo[2,1- c][1,4]oxazin-6-yl)methoxy)-8,9-dihydro- 10H-7-oxa-1,3,6,10- tetraazacyclohepta[de]naphthalen-10- yl)ethyl)pyridin-2-amine |
| 17 | 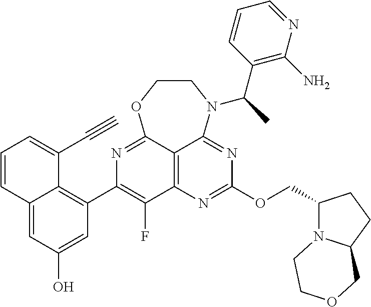 [SMILES: C#Cc1cccc2cc(O)cc(-c3nc4c5c(nc(OC[C@@H]6CC[C@H]7COCCN76)nc5c3F)N([C@H](C)c3cccnc3N)CCO4)c12] | 4-(10-((R)-1-(2-aminopyridin-3-yl)ethyl)- 4-fluoro-2-(((6S,8aS)-hexahydro-1H- pyrrolo[2,1-c][1,4]oxazin-6-yl)methoxy)- 9,10-dihydro-8H-7-oxa-1,3,6,10- tetraazacyclohepta[de]naphthalen-5-yl)- 5-ethynylnaphthalen-2-ol |
| 18 | 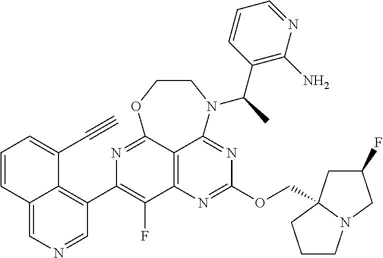 [SMILES: C#Cc1cccc2cncc(-c3nc4c5c(nc(OC[C@@]67CCCN6C[C@H](F)C7)nc5c3F)N([C@H](C)c3cccnc3N)CCO4)c12] | 3-((R)-1-(5-(5-ethynylisoquinolin-4-yl)-4- fluoro-2-(((2R,7aS)-2-fluorotetrahydro- 1H-pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 19 | 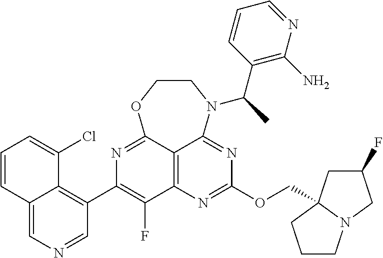 [SMILES: C[C@H](c1cccnc1N)N1CCOc2nc(-c3cncc4cccc(Cl)c34)c(F)c3nc(OC[C@@]45CCCN4C[C@H](F)C5)nc1c23] | 3-((R)-1-(5-(5-chloroisoquinolin-4-yl)-4- fluoro-2-(((2R,7aS)-2-fluorotetrahydro- 1H-pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 20 | 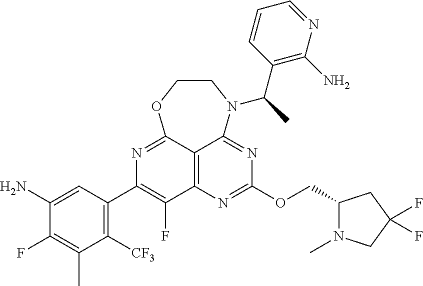 [SMILES: Cc1c(F)c(N)cc(-c2nc3c4c(nc(OC[C@@H]5CC(F)(F)CN5C)nc4c2F)N([C@H](C)c2cccnc2N)CCO3)c1C(F)(F)F] | 3-((R)-1-(5-(5-amino-4-fluoro-3-methyl-2- (trifluoromethyl)phenyl)-2-(((S)-4,4- difluoro-1-methylpyrrolidin-2-yl)methoxy)- 4-fluoro-8,9-dihydro-10H-7-oxa-1,3,6,10- tetraazacyclohepta[de]naphthalen-10- yl)ethyl)pyridin-2-amine |
| 21A | 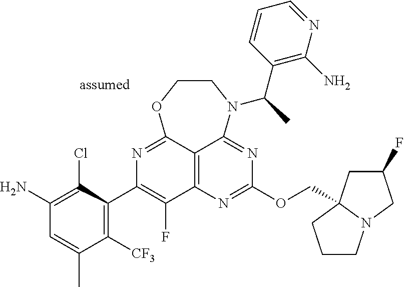 | 3-((R)-1-((R)-5-(3-amino-2-chloro-5- methyl-6-(trifluoromethyl)phenyl)-4- fluoro-2-(((2R,7aS)-2-fluorotetrahydro- 1H-pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 21B | 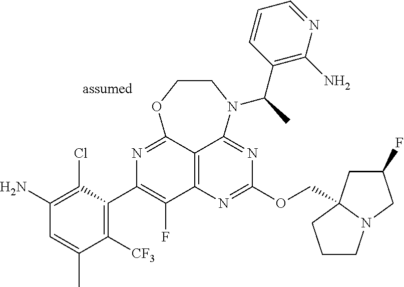 | 3-((R)-1-((S)-5-(3-amino-2-chloro-5- methyl-6-(trifluoromethyl)phenyl)-4- fluoro-2-(((2R,7aS)-2-fluorotetrahydro- 1H-pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 22 | 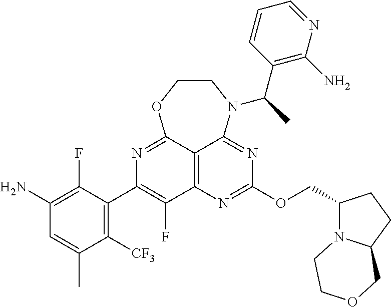 [SMILES: Cc1cc(N)c(F)c(-c2nc3c4c(nc(OC[C@@H]5CC[C@H]6COCCN65)nc4c2F)N([C@H](C)c2cccnc2N)CCO3)c1C(F)(F)F] | 3-((1R)-1-(5-(3-amino-2-fluoro-5-methyl- 6-(trifluoromethyl)phenyl)-4-fluoro-2- (((6S,8aS)-hexahydro-1H-pyrrolo[2,1- c][1,4]oxazin-6-yl)methoxy)-8,9-dihydro- 10H-7-oxa-1,3,6,10- tetraazacyclohepta[de]naphthalen-10- yl)ethyl)pyridin-2-amine |
| 23 | 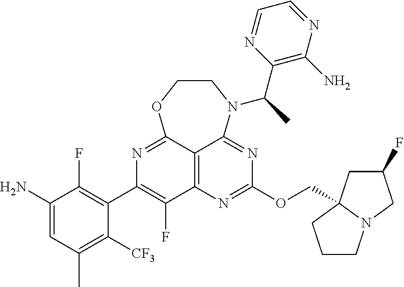 [SMILES: Cc1cc(N)c(F)c(-c2nc3c4c(nc(OC[C@@]56CCCN5C[C@H](F)C6)nc4c2F)N([C@H](C)c2nccnc2N)CCO3)c1C(F)(F)F] | 3-((1R)-1-(5-(3-amino-2-fluoro-5-methyl- 6-(trifluoromethyl)phenyl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyrazin-2-amine |
| 24 | 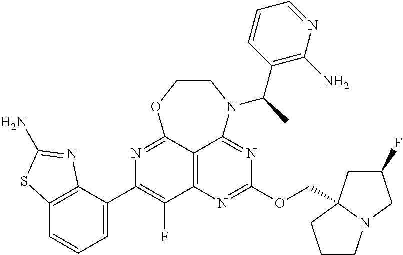 [SMILES: C[C@H](c1cccnc1N)N1CCOc2nc(-c3cccc4sc(N)nc34)c(F)c3nc(OC[C@@]45CCCN4C[C@H](F)C5)nc1c23] | 4-(10-((R)-1-(2-aminopyridin-3-yl)ethyl)- 4-fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5- yl)benzo[d]thiazol-2-amine |
| 25 | 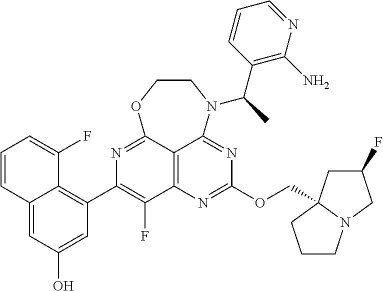 [SMILES: C[C@H](c1cccnc1N)N1CCOc2nc(-c3cc(O)cc4cccc(F)c34)c(F)c3nc(OC[C@@]45CCCN4C[C@H](F)C5)nc1c23] | 4-(10-((R)-1-(2-aminopyridin-3-yl)ethyl)- 4-fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5-yl)- 5-fluoronaphthalen-2-ol |
| 26 | 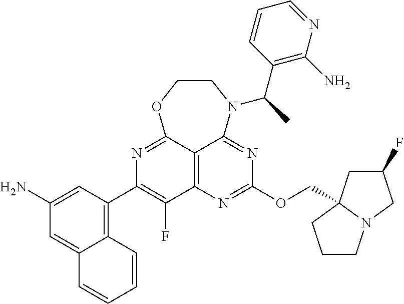 [SMILES: C[C@H](c1cccnc1N)N1CCOc2nc(-c3cc(N)cc4ccccc34)c(F)c3nc(OC[C@@]45CCCN4C[C@H](F)C5)nc1c23] | 4-(10-((R)-1-(2-aminopyridin-3-yl)ethyl)- 4-fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5- yl)naphthalen-2-ol |
| 27 | 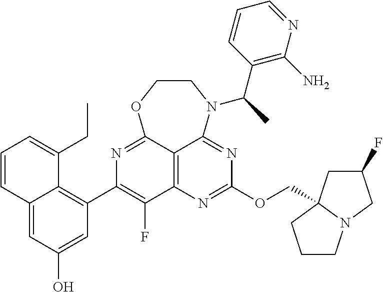 [SMILES: CCc1cccc2cc(O)cc(-c3nc4c5c(nc(OC[C@@]67CCCN6C[C@H](F)C7)nc5c3F)N([C@H](C)c3cccnc3N)CCO4)c12] | 4-(10-((R)-1-(2-aminopyridin-3-yl)ethyl)- 4-fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5-yl)- 5-ethylnaphthalen-2-ol |
| 28A | 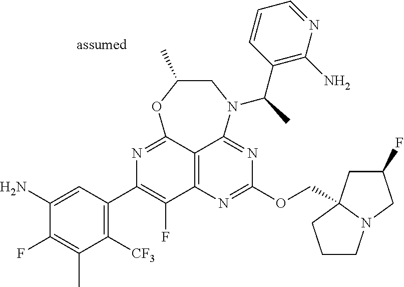 [SMILES: Cc1c(F)c(N)cc(-c2nc3c4c(nc(OC[C@@]56CCCN5C[C@H](F)C6)nc4c2F)N([C@H](C)c2cccnc2N)C[C@@H](C)O3)c1C(F)(F)F] | 3-((R)-1-((R)-5-(5-amino-4-fluoro-3- methyl-2-(trifluoromethyl)phenyl)-4- fluoro-2-(((2R,7aS)-2-fluorotetrahydro- 1H-pyrrolizin-7a(5H)-yl)methoxy)-8- methyl-8,9-dihydro-10H-7-oxa-1,3,6,10- tetraazacyclohepta[de]naphthalen-10- yl)ethyl)pyridin-2-amine |
| 28B | 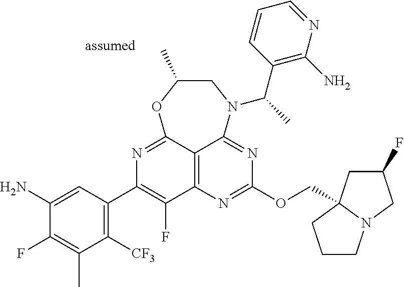 [SMILES: Cc1c(F)c(N)cc(-c2nc3c4c(nc(OC[C@@]56CCCN5C[C@H](F)C6)nc4c2F)N([C@@H](C)c2cccnc2N)C[C@@H](C)O3)c1C(F)(F)F] | 3-((S)-1-((R)-5-(5-amino-4-fluoro-3- methyl-2-(trifluoromethyl)phenyl)-4- fluoro-2-(((2R,7aS)-2-fluorotetrahydro- 1H-pyrrolizin-7a(5H)-yl)methoxy)-8- methyl-8,9-dihydro-10H-7-oxa-1,3,6,10- tetraazacyclohepta[de]naphthalen-10- yl)ethyl)pyridin-2-amine |
| 28C | 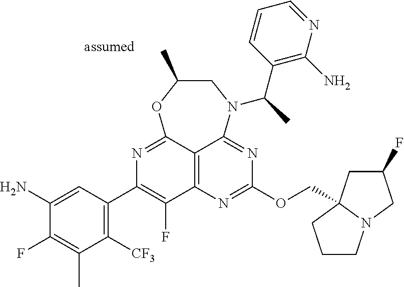 [SMILES: Cc1c(F)c(N)cc(-c2nc3c4c(nc(OC[C@@]56CCCN5C[C@H](F)C6)nc4c2F)N([C@H](C)c2cccnc2N)C[C@H](C)O3)c1C(F)(F)F] | 3-((R)-1-((S)-5-(5-amino-4-fluoro-3- methyl-2-(trifluoromethyl)phenyl)-4- fluoro-2-(((2R,7aS)-2-fluorotetrahydro- 1H-pyrrolizin-7a(5H)-yl)methoxy)-8- methyl-8,9-dihydro-10H-7-oxa-1,3,6,10- tetraazacyclohepta[de]naphthalen-10- yl)ethyl)pyridin-2-amine |
| 28D | 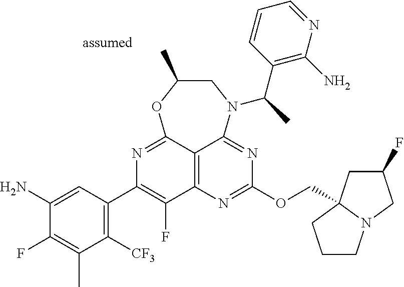 [SMILES: Cc1c(F)c(N)cc(-c2nc3c4c(nc(OC[C@@]56CCCN5C[C@H](F)C6)nc4c2F)N([C@H](C)c2cccnc2N)C[C@H](C)O3)c1C(F)(F)F] | 3-((S)-1-((S)-5-(5-amino-4-fluoro-3- methyl-2-(trifluoromethyl)phenyl)-4- fluoro-2-(((2R,7aS)-2-fluorotetrahydro- 1H-pyrrolizin-7a(5H)-yl)methoxy)-8- methyl-8,9-dihydro-10H-7-oxa-1,3,6,10- tetraazacyclohepta[de]naphthalen-10- yl)ethyl)pyridin-2-amine |
| 29 | 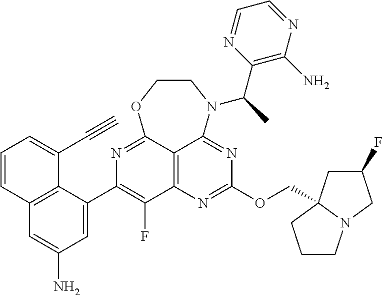 [SMILES: C#Cc1cccc2cc(N)cc(-c3nc4c5c(nc(OC[C@@]67CCCN6C[C@H](F)C7)nc5c3F)N([C@H](C)c3nccnc3N)CCO4)c12] | 3-((R)-1-(5-(3-Amino-8- ethynylnaphthalen-1-yl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyrazin-2-amine |
| 30 | 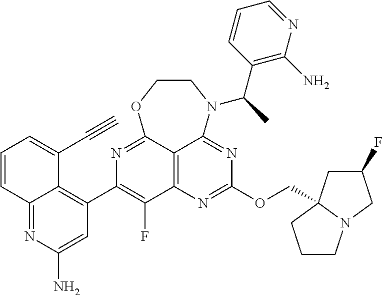 [SMILES: C#Cc1cccc2nc(N)cc(-c3nc4c5c(nc(OC[C@@]67CCCN6C[C@H](F)C7)nc5c3F)N([C@H](C)c3cccnc3N)CCO4)c12] | 4-(10-((R)-1-(2-aminopyridin-3-yl)ethyl)- 4-fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5-yl)- 5-ethynylquinolin-2-amine |
| 31 | 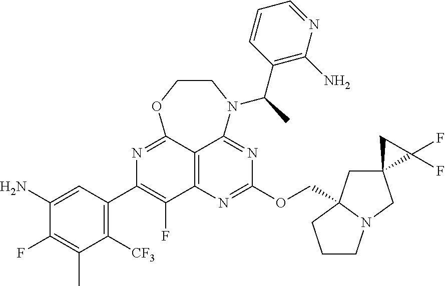 [SMILES: Cc1c(F)c(N)cc(-c2nc3c4c(nc(OC[C@@]56CCCN5C[C@]5(CC5(F)F)C6)nc4c2F)N([C@H](C)c2cccnc2N)CCO3)c1C(F)(F)F] | 3-((R)-1-(5-(5-amino-4-fluoro-3-methyl-2- (trifluoromethyl)phenyl)-2-(((1R,7a′S)- 2,2-difluorodihydro-1′H, 3′H- spiro[cyclopropane-1,2′-pyrrolizin]- 7a′(5′H)-yl)methoxy)-4-fluoro-8,9-dihydro- 10H-7-oxa-1,3,6,10- tetraazacyclohepta[de]naphthalen-10- yl)ethyl)pyridin-2-amine |
| 32 | 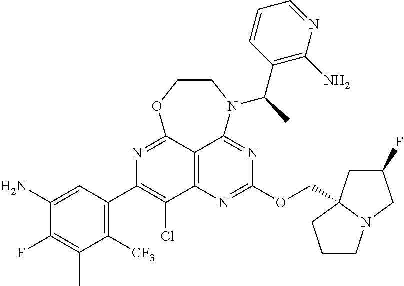 [SMILES: Cc1c(F)c(N)cc(-c2nc3c4c(nc(OC[C@@]56CCCN5C[C@H](F)C6)nc4c2Cl)N([C@H](C)c2cccnc2N)CCO3)c1C(F)(F)F] | 3-((R)-1-(5-(5-amino-4-fluoro-3-methyl-2- (trifluoromethyl)phenyl)-4-chloro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyridin-2-amine |

[symbol]

indicates data missing or illegible when filed

In one embodiment of the compounds or a stereoisomer, atropisomer, tautomer, or pharmaceutically acceptable salt thereof of formula (I), (Ia), (11), (IIa), (IIb), (IIc), (III), (IIIa), (IIIb), (IIIc), (IV), (IVa), (IVb), (IVc), (V), (Va), (Vb), or (Vc) described herein, the compound is one set forth in Table 2 below.

TABLE 2
Representative Compounds

| Ex. | | |
|---|---|---|
| No. | Structure | Name |
| 505 | 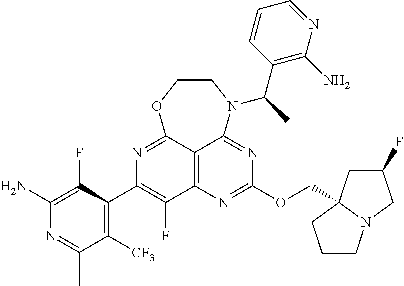 | 4-((R)-10-((R)-1-(2-aminopyridin-3- yl)ethyl)-4-fluoro-2-(((2R,7aS)-2- fluorotetrahydro-1H-pyrrolizin-7a(5H)- yl)methoxy)-9,10-dihydro-8H-7-oxa- 1,3,6,10- tetraazacyclohepta[de]naphthalen-5-yl)-3- fluoro-6-methyl-5-(trifluoromethyl)pyridin- 2-amine |
| 506 | 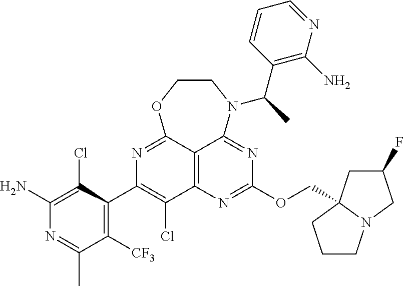 | 4-((R)-10-((R)-1-(2-aminopyridin-3- yl)ethyl)-4-chloro-2-(((2R,7aS)-2- fluorotetrahydro-1H-pyrrolizin-7a(5H)- yl)methoxy)-9,10-dihydro-8H-7-oxa- 1,3,6,10- tetraazacyclohepta[de]naphthalen-5-yl)-3- chloro-6-methyl-5-(trifluoromethyl)pyridin- 2-amine |
| 508 | 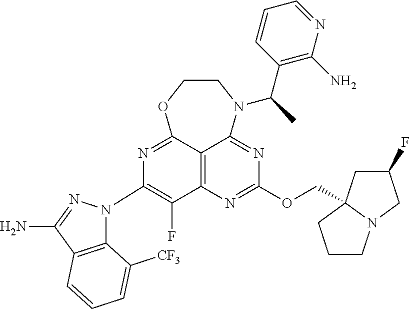 [SMILES: C[C@H](c1cccnc1N)N1CCOc2nc(-n3nc(N)c4cccc(C(F)(F)F)c43)c(F)c3nc(OC[C@@]45CCCN4C[C@H](F)C5)nc1c23] | 1-(10-((R)-1-(2-aminopyridin-3-yl)ethyl)-4- fluoro-2-(((2R,7aS)-2-fluorotetrahydro-1H- pyrrolizin-7a(5H)-yl)methoxy)-9,10- dihydro-8H-7-oxa-1,3,6,10- tetraazacyclohepta[de]naphthalen-5-yl)-7- (trifluoromethyl)-1H-indazol-3-amine |

In one embodiment of the compounds or a stereoisomer, atropisomer, tautomer, or pharmaceutically acceptable salt thereof of formula (I), (Ia), (11), (IIa), (IIb), (IIc), (III), (IIIa), (IIIb), (IIIc), (IV), (IVa), (IVb), (IVc), (V), (Va), (Vb), or (Vc) described herein, the compound is one set forth in Table 3 below.

TABLE 3
Further Representative Compounds

| Ex. | | |
|---|---|---|
| No. | Structure | ¹H NMR |
| 601a | 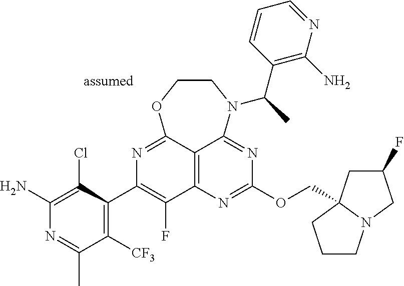 | 4-((R)-10-((R)-1-(2-aminopyridin-3- yl)ethyl)-4-fluoro-2-(((2R,7aS)-2- fluorotetrahydro-1H-pyrrolizin-7a(5H)- yl)methoxy)-9,10-dihydro-8H-7-oxa- 1,3,6,10- tetraazacyclohepta[de]naphthalen-5-yl)-3- chloro-6-methyl-5-(trifluoromethyl)pyridin- 2-amine |
| 601b | 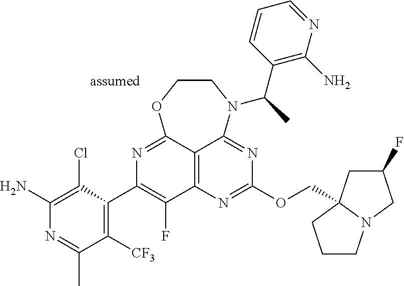 | 4-((S)-10-((R)-1-(2-aminopyridin-3- yl)ethyl)-4-fluoro-2-(((2R,7aS)-2- fluorotetrahydro-1H-pyrrolizin-7a(5H)- yl)methoxy)-9,10-dihydro-8H-7-oxa- 1,3,6,10- tetraazacyclohepta[de]naphthalen-5-yl)-3- chloro-6-methyl-5-(trifluoromethyl)pyridin- 2-amine |
| 602 | 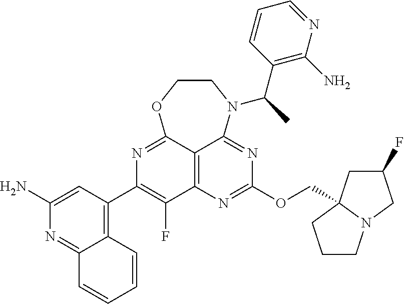 [SMILES: C[C@H](c1cccnc1N)N1CCOc2nc(-c3cc(N)nc4ccccc34)c(F)c3nc(OC[C@@]45CCCN4C[C@H](F)C5)nc1c23] | 4-(10-((R)-1-(2-aminopyridin-3-yl)ethyl)-4- fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5- yl)quinolin-2-amine |
| 604 | 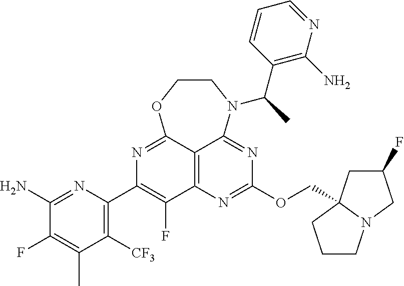 [SMILES: Cc1c(F)c(N)nc(-c2nc3c4c(nc(OC[C@@]56CCCN5C[C@H](F)C6)nc4c2F)N([C@H](C)c2cccnc2N)CCO3)c1C(F)(F)F] | 6-(10-((R)-1-(2-aminopyridin-3-yl)ethyl)-4- fluoro-2-(((2R,7aS)-2-fluorotetrahydro-1H- pyrrolizin-7a(5H)-yl)methoxy)-9,10-dihydro- 8H-7-oxa-1,3,6,10- tetraazacyclohepta[de]naphthalen-5-yl)-3- fluoro-4-methyl-5-(trifluoromethyl)pyridin-2- amine |
| 605 | 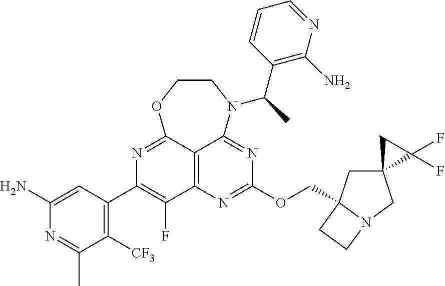 [SMILES: Cc1nc(N)cc(-c2nc3c4c(nc(OC[C@@]56CCN5C[C@]5(CC5(F)F)C6)nc4c2F)N([C@H](C)c2cccnc2N)CCO3)c1C(F)(F)F] | 4-(10-((R)-1-(2-aminopyridin-3-yl)ethyl)-2- (((3R,5R)-2′,2′-difluoro-1- azaspiro[bicyclo[3.2.0]heptane-3,1′- cyclopropan]-5-yl)methoxy)-4-fluoro-9,10- dihydro-8H-7-oxa-1,3,6,10- tetraazacyclohepta[de]naphthalen-5-yl)-6- methyl-5-(trifluoromethyl)pyridin-2-amine |
| 606 | 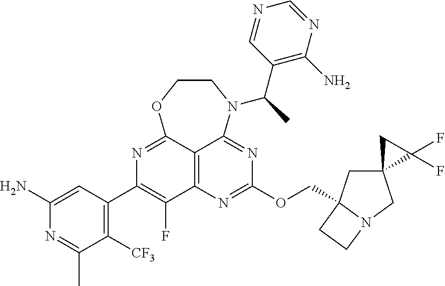 [SMILES: Cc1nc(N)cc(-c2nc3c4c(nc(OC[C@@]56CCN5C[C@]5(CC5(F)F)C6)nc4c2F)N([C@H](C)c2cncnc2N)CCO3)c1C(F)(F)F] | 5-((R)-1-(5-(6-amino-2-methyl-3- (trifluoromethyl)pyridin-4-yl)-2-(((3R,5R)- 2′,2′-difluoro-1- azaspiro[bicyclo[3.2.0]heptane-3,1′- cyclopropan]-5-yl)methoxy)-4-fluoro-8,9- dihydro-10H-cyclohepta[de]naphthalen-10- yl)ethyl)pyrimidin-4-amine |
| 607 | 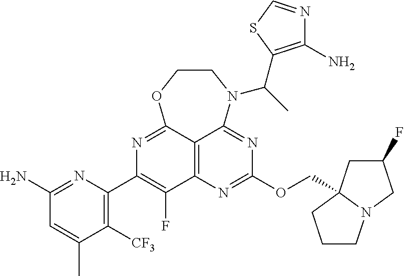 [SMILES: Cc1cc(N)nc(-c2nc3c4c(nc(OC[C@@]56CCCN5C[C@H](F)C6)nc4c2F)N(C(C)c2scnc2N)CCO3)c1C(F)(F)F] | 5-(1-(5-(6-amino-4-methyl-3- (trifluoromethyl)pyridin-2-yl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)thiazol-4-amine |
| 608 | 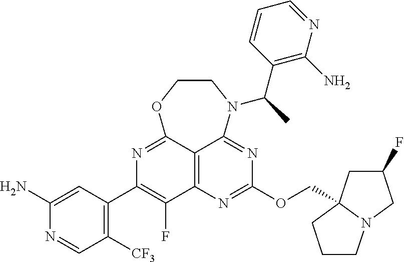 [SMILES: C[C@H](c1cccnc1N)N1CCOc2nc(-c3cc(N)ncc3C(F)(F)F)c(F)c3nc(OC[C@@]45CCCN4C[C@H](F)C5)nc1c23] | 4-(10-((R)-1-(2-aminopyridin-3-yl)ethyl)-4- fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5-yl)-5- (trifluoromethyl)pyridin-2-amine |
| 609 | 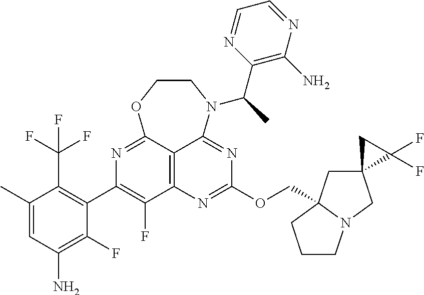 [SMILES: Cc1cc(N)c(F)c(-c2nc3c4c(nc(OC[C@@]56CCCN5C[C@]5(CC5(F)F)C6)nc4c2F)N([C@H](C)c2nccnc2N)CCO3)c1C(F)(F)F] | 3-((1R)-1-(5-(3-amino-2-fluoro-5-methyl- 6-(trifluoromethyl)phenyl)-2-(((1R,7a′S)- 2,2-difluorodihydro-1′H, 3′H- spiro[cyclopropane-1,2′-pyrrolizin]- 7a′(5′H)-yl)methoxy)-4-fluoro-8,9-dihydro- 10H-cyclohepta[de]naphthalen-10- yl)ethyl)pyrazin-2-amine |
| 612 | 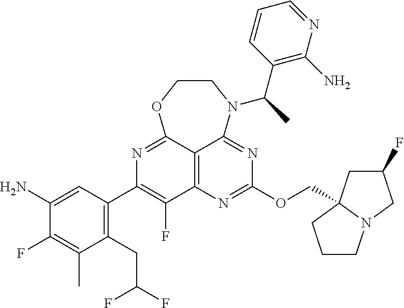 [SMILES: Cc1c(F)c(N)cc(-c2nc3c4c(nc(OC[C@@]56CCCN5C[C@H](F)C6)nc4c2F)N([C@H](C)c2cccnc2N)CCO3)c1CC(F)F] | 3-((R)-1-(5-(5-amino-2-(2,2-difluoroethyl)- 4-fluoro-3-methylphenyl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 613 | 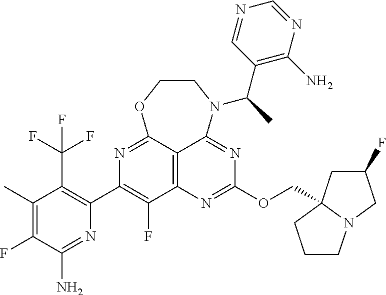 [SMILES: Cc1c(F)c(N)nc(-c2nc3c4c(nc(OC[C@@]56CCCN5C[C@H](F)C6)nc4c2F)N([C@H](C)c2cncnc2N)CCO3)c1C(F)(F)F] | 5-((R)-1-(5-(6-amino-5-fluoro-4-methyl-3- (trifluoromethyl)pyridin-2-yl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyrimidin-4-amine |
| 614 | 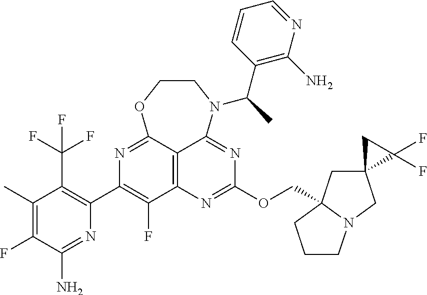 [SMILES: Cc1c(F)c(N)nc(-c2nc3c4c(nc(OC[C@@]56CCCN5C[C@]5(CC5(F)F)C6)nc4c2F)N([C@H](C)c2cccnc2N)CCO3)c1C(F)(F)F] | 6-(10-((R)-1-(2-aminopyridin-3-yl)ethyl)-2- (((1R,7a′S)-2,2-difluorodihydro-1′H,3′H- spiro[cyclopropane-1,2′-pyrrolizin]- 7a′(5′H)-yl)methoxy)-4-fluoro-9,10- dihydro-8H-cyclohepta[de]naphthalen-5- yl)-3-fluoro-4-methyl-5- (trifluoromethyl)pyridin-2-amine |
| 615 | 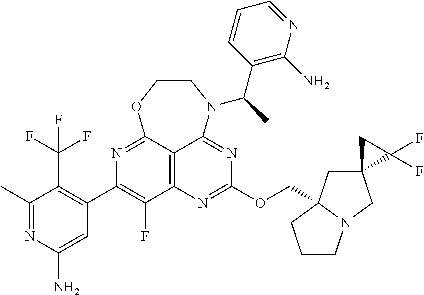 [SMILES: Cc1nc(N)cc(-c2nc3c4c(nc(OC[C@@]56CCCN5C[C@]5(CC5(F)F)C6)nc4c2F)N([C@H](C)c2cccnc2N)CCO3)c1C(F)(F)F] | 4-(10-((R)-1-(2-aminopyridin-3-yl)ethyl)-2- (((1R,7a′S)-2,2-difluorodihydro-1′H,3′H- spiro[cyclopropane-1,2′-pyrrolizin]- 7a′(5′H)-yl)methoxy)-4-fluoro-9,10- dihydro-8H-cyclohepta[de]naphthalen-5- yl)-6-methyl-5-(trifluoromethyl)pyridin-2- amine |
| 616 | 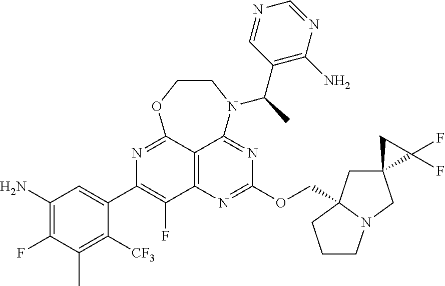 [SMILES: Cc1c(F)c(N)cc(-c2nc3c4c(nc(OC[C@@]56CCCN5C[C@]5(CC5(F)F)C6)nc4c2F)N([C@H](C)c2cncnc2N)CCO3)c1C(F)(F)F] | 5-((R)-1-(5-(5-amino-4-fluoro-3-methyl-2- (trifluoromethyl)phenyl)-2-(((1R,7a′S)-2,2- difluorodihydro-1′H,3′H-spiro[cyclopropane- 1,2′-pyrrolizin]-7a′(5′H)-yl)methoxy)-4- fluoro-8,9-dihydro-10H- cyclohepta[de]naphthalen-10- yl)ethyl)pyrimidin-4-amine |
| 617 | 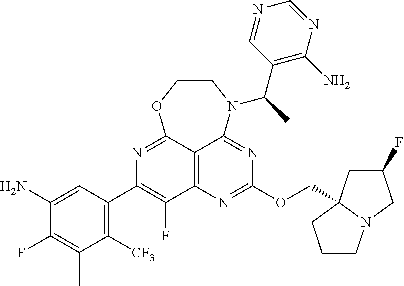 [SMILES: Cc1c(F)c(N)cc(-c2nc3c4c(nc(OC[C@@]56CCCN5C[C@H](F)C6)nc4c2F)N([C@H](C)c2cncnc2N)CCO3)c1C(F)(F)F] | 5-((R)-1-(5-(5-amino-4-fluoro-3-methyl-2- (trifluoromethyl)phenyl)-4-fluoro-2- (((2R,7aS)-2-fluorotetrahydro-1H-pyrrolizin- 7a(5H)-yl)methoxy)-8,9-dihydro-10H-7-oxa- 1,3,6,10-tetraazacyclohepta[de]naphthalen- 10-yl)ethyl)pyrimidin-4-amine |
| 618 | 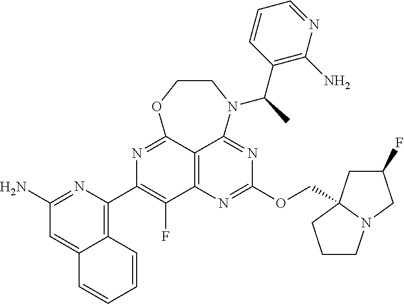 [SMILES: C[C@H](c1cccnc1N)N1CCOc2nc(-c3nc(N)cc4ccccc34)c(F)c3nc(OC[C@@]45CCCN4C[C@H](F)C5)nc1c23] | 1-(10-((R)-1-(2-aminopyridin-3-yl)ethyl)-4- fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5- yl)isoquinolin-3-amine |
| 620 | 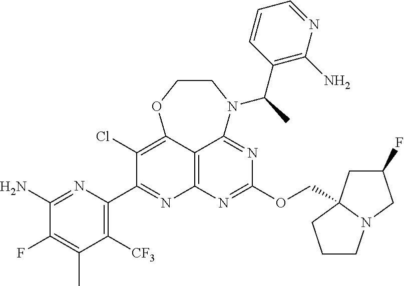 [SMILES: Cc1c(F)c(N)nc(-c2nc3nc(OC[C@@]45CCCN4C[C@H](F)C5)nc4c3c(c2Cl)OCCN4[C@H](C)c2cccnc2N)c1C(F)(F)F] | 6-(10-((R)-1-(2-aminopyridin-3-yl)ethyl)-4- chloro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5-yl)-3- fluoro-4-methyl-5-(trifluoromethyl)pyridin- 2-amine |
| 621 | 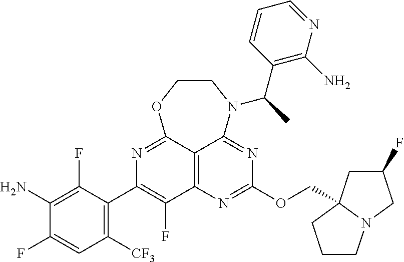 [SMILES: C[C@H](c1cccnc1N)N1CCOc2nc(-c3c(C(F)(F)F)cc(F)c(N)c3F)c(F)c3nc(OC[C@@]45CCCN4C[C@H](F)C5)nc1c23] | 3-((1R)-1-(5-(3-amino-2,4-difluoro-6- (trifluoromethyl)phenyl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 622a | 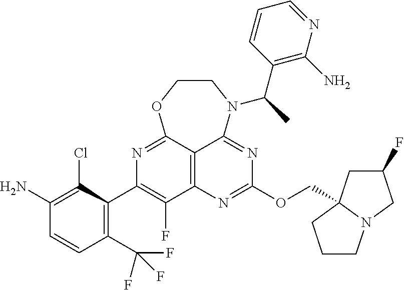 | 3-((R)-1-((R)-5-(3-amino-2-chloro-6- (trifluoromethyl)phenyl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 622b | 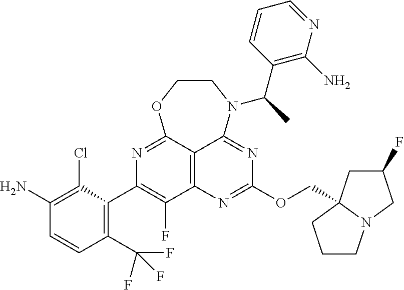 | 3-((R)-1-((S)-5-(3-amino-2-chloro-6- (trifluoromethyl)phenyl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 623 | 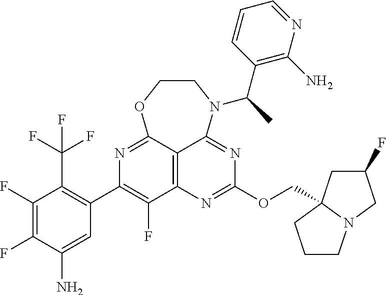 [SMILES: C[C@H](c1cccnc1N)N1CCOc2nc(-c3cc(N)c(F)c(F)c3C(F)(F)F)c(F)c3nc(OC[C@@]45CCCN4C[C@H](F)C5)nc1c23] | 3-((R)-1-(5-(5-amino-3,4-difluoro-2- (trifluoromethyl)phenyl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 624 | 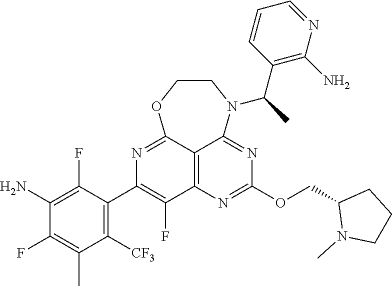 [SMILES: Cc1c(F)c(N)c(F)c(-c2nc3c4c(nc(OC[C@@H]5CCCN5C)nc4c2F)N([C@H](C)c2cccnc2N)CCO3)c1C(F)(F)F] | 3-((1R)-1-(5-(3-amino-2,4-difluoro-5- methyl-6-(trifluoromethyl)phenyl)-4-fluoro- 2-(((S)-1-methylpyrrolidin-2-yl)methoxy)- 8,9-dihydro-10H-7-oxa-1,3,6,10- tetraazacyclohepta[de]naphthalen-10- yl)ethyl)pyridin-2-amine |
| 626 | 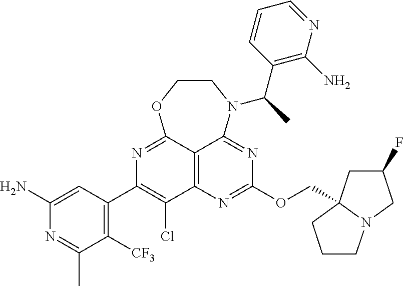 [SMILES: Cc1nc(N)cc(-c2nc3c4c(nc(OC[C@@]56CCCN5C[C@H](F)C6)nc4c2Cl)N([C@H](C)c2cccnc2N)CCO3)c1C(F)(F)F] | 4-(10-((R)-1-(2-aminopyridin-3-yl)ethyl)-4- chloro-2-(((2R,7aS)-2-fluorotetrahydro-1H- pyrrolizin-7a(5H)-yl)methoxy)-9,10-dihydro- 8H-7-oxa-1,3,6,10- tetraazacyclohepta[de]naphthalen-5-yl)-6- methyl-5-(trifluoromethyl)pyridin-2-amine |
| 627 | 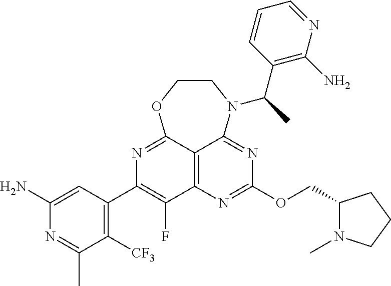 [SMILES: Cc1nc(N)cc(-c2nc3c4c(nc(OC[C@@H]5CCCN5C)nc4c2F)N([C@H](C)c2cccnc2N)CCO3)c1C(F)(F)F] | 4-(10-((R)-1-(2-aminopyridin-3-yl)ethyl)-4- fluoro-2-(((S)-1-methylpyrrolidin-2- yl)methoxy)-9,10-dihydro-8H-7-oxa- 1,3,6,10- tetraazacyclohepta[de]naphthalen-5-yl)-6- methyl-5-(trifluoromethyl)pyridin-2-amine |
| 628 | 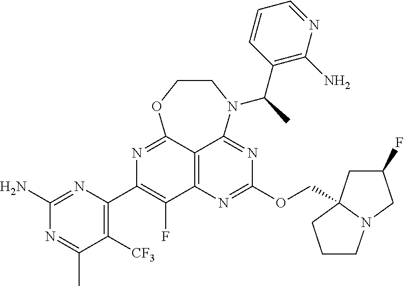 [SMILES: Cc1nc(N)nc(-c2nc3c4c(nc(OC[C@@]56CCCN5C[C@H](F)C6)nc4c2F)N([C@H](C)c2cccnc2N)CCO3)c1C(F)(F)F] | 4-(10-((R)-1-(2-aminopyridin-3-yl)ethyl)-4- fluoro-2-(((2R,7aS)-2-fluorotetrahydro- 1H-pyrrolizin-7a(5H)-yl)methoxy)-9,10- dihydro-8H-7-oxa-1,3,6,10- tetraazacyclohepta[de]naphthalen-5-yl)-6- methyl-5-(trifluoromethyl)pyrimidin-2- amine |
| 629a | 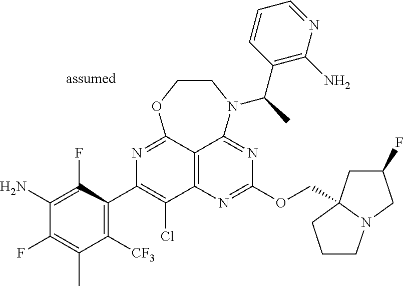 | 3-((R)-1-((R)-5-(3-amino-2,4-difluoro-5- methyl-6-(trifluoromethyl)phenyl)-4- chloro-2-(((2R,7aS)-2-fluorotetrahydro- 1H-pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 629b | 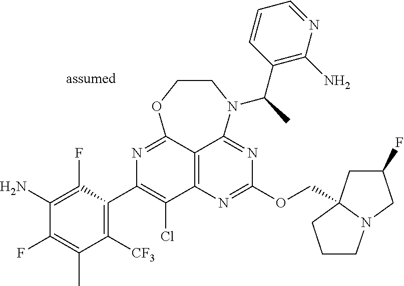 | 3-((R)-1-((S)-5-(3-amino-2,4-difluoro-5- methyl-6-(trifluoromethyl)phenyl)-4- chloro-2-(((2R,7aS)-2-fluorotetrahydro- 1H-pyrrolizin-7a(5H)-yl)methoxy)-8,9- dihydro-10H-7-oxa-1,3,6,10- tetraazacyclohepta[de]naphthalen-10- yl)ethyl)pyridin-2-amine |
| 630 | 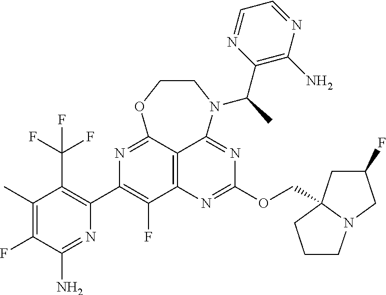 [SMILES: Cc1c(F)c(N)nc(-c2nc3c4c(nc(OC[C@@]56CCCN5C[C@H](F)C6)nc4c2F)N([C@H](C)c2nccnc2N)CCO3)c1C(F)(F)F] | 3-((R)-1-(5-(6-amino-5-fluoro-4-methyl-3- (trifluoromethyl)pyridin-2-yl)-4-fluoro-2- (((2R,7aS)-2-fluorotetrahydro-1H- pyrrolizin-7a(5H)-yl)methoxy)-8,9- dihydro-10H-7-oxa-1,3,6,10- tetraazacyclohepta[de]naphthalen-10- yl)ethyl)pyrazin-2-amine |
| 631 | 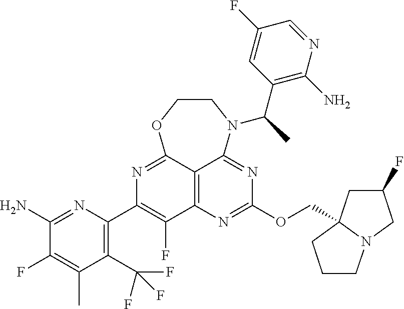 [SMILES: Cc1c(F)c(N)nc(-c2nc3c4c(nc(OC[C@@]56CCCN5C[C@H](F)C6)nc4c2F)N([C@H](C)c2cc(F)cnc2N)CCO3)c1C(F)(F)F] | 6-(10-((R)-1-(2-amino-5-fluoropyridin-3- yl)ethyl)-4-fluoro-2-(((2R,7aS)-2- fluorotetrahydro-1H-pyrrolizin-7a(5H)- yl)methoxy)-9,10-dihydro-8H-7-oxa- 1,3,6,10- tetraazacyclohepta[de]naphthalen-5-yl)-3- fluoro-4-methyl-5-(trifluoromethyl)pyridin- 2-amine |
| 632 | 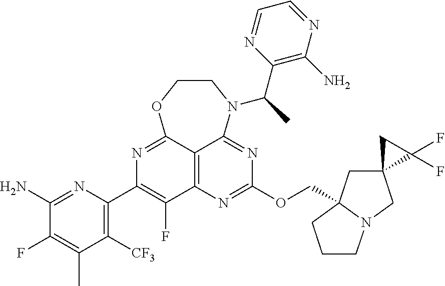 [SMILES: Cc1c(F)c(N)nc(-c2nc3c4c(nc(OC[C@@]56CCCN5C[C@]5(CC5(F)F)C6)nc4c2F)N([C@H](C)c2nccnc2N)CCO3)c1C(F)(F)F] | 3-((R)-1-(5-(6-amino-5-fluoro-4-methyl-3- (trifluoromethyl)pyridin-2-yl)-2- (((1R,7a′S)-2,2-difluorodihydro-1′H,3′H- spiro[cyclopropane-1,2′-pyrrolizin]- 7a′(5′H)-yl)methoxy)-4-fluoro-8,9-dihydro- 10H-cyclohepta[de]naphthalen-10- yl)ethyl)pyrazin-2-amine |
| 633 | 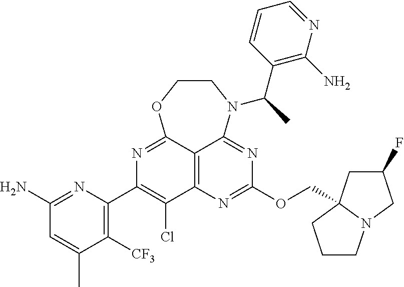 [SMILES: Cc1cc(N)nc(-c2nc3c4c(nc(OC[C@@]56CCCN5C[C@H](F)C6)nc4c2Cl)N([C@H](C)c2cccnc2N)CCO3)c1C(F)(F)F] | 6-(10-((R)-1-(2-aminopyridin-3-yl)ethyl)-4- chloro-2-(((2R,7aS)-2-fluorotetrahydro-1H- pyrrolizin-7a(5H)-yl)methoxy)-9,10-dihydro- 8H-7-oxa-1,3,6,10- tetraazacyclohepta[de]naphthalen-5-yl)-4- methyl-5-(trifluoromethyl)pyridin-2-amine |
| 634 | 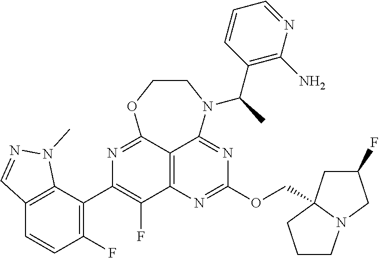 [SMILES: C[C@H](c1cccnc1N)N1CCOc2nc(-c3c(F)ccc4cnn(C)c34)c(F)c3nc(OC[C@@]45CCCN4C[C@H](F)C5)nc1c23] | 3-((1R)-1-(4-fluoro-5-(6-fluoro-1-methyl- 1H-indazol-7-yl)-2-(((2R,7aS)-2- fluorotetrahydro-1H-pyrrolizin-7a(5H)- yl)methoxy)-8,9-dihydro-10H-7-oxa- 1,3,6,10- tetraazacyclohepta[de]naphthalen-10- yl)ethyl)pyridin-2-amine |
| 635 | 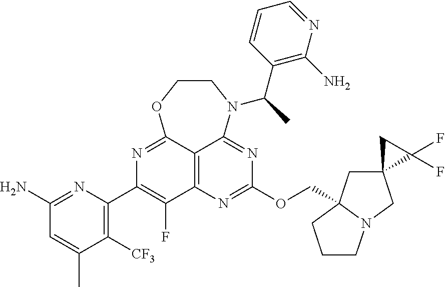 [SMILES: Cc1cc(N)nc(-c2nc3c4c(nc(OC[C@@]56CCCN5C[C@]5(CC5(F)F)C6)nc4c2F)N([C@H](C)c2cccnc2N)CCO3)c1C(F)(F)F] | 6-(10-((R)-1-(2-aminopyridin-3-yl)ethyl)-2- (((1R,7a′S)-2,2-difluorodihydro-1′H,3′H- spiro[cyclopropane-1,2′-pyrrolizin]- 7a′(5′H)-yl)methoxy)-4-fluoro-9,10- dihydro-8H-cyclohepta[de]naphthalen-5- yl)-4-methyl-5-(trifluoromethyl)pyridin-2- amine |

### SYNTHESIS OF COMPOUNDS

Compounds or a stereoisomer, atropisomer, tautomer, or pharmaceutically acceptable salt thereof as described herein of the present disclosure can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, vol. 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds.) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, vol. 1-40. The synthetic reaction schemes provided herein are merely illustrative of some methods by which the compounds or pharmaceutical acceptable salts thereof described herein can be synthesized, and various modifications to these synthetic reaction schemes can be made and will be suggested to one skilled in the art having referred to the disclosure contained herein.

Synthetic chemistry transformations and protecting group methodologies (protection and deprotection) useful in synthesizing compounds described herein and necessary reagents and intermediates include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds or a stereoisomer, atropisomer, tautomer, or pharmaceutically acceptable salt thereof as described herein described herein can be prepared singly or as compound libraries comprising at least 2, for Example 65 to 1,000 compounds, or 10 to 100 compounds. Libraries of compounds or a stereoisomer, atropisomer, tautomer, or pharmaceutically acceptable salt thereof as described herein of the formulae described herein can be prepared by a combinatorial split and mix approach or by multiple parallel syntheses using, for example, either solution phase or solid phase chemistry. Thus according to a further aspect provided herein is a compound library comprising at least 2 compounds or a stereoisomer, atropisomer, tautomer, or pharmaceutically acceptable salt thereof as described herein.

The Examples provide exemplary methods for preparing compounds or a stereoisomer, atropisomer, tautomer, or pharmaceutically acceptable salt thereof as described herein. Those skilled in the art will appreciate that other synthetic routes can be used to synthesize the compounds or a stereoisomer, atropisomer, tautomer, or pharmaceutically acceptable salt thereof as described herein described herein. Although specific starting materials and reagents are depicted and discussed in the Examples, other starting materials and reagents can be substituted to provide a variety of derivatives and/or reaction conditions. In addition, many of the exemplary compounds prepared by the described methods can be further modified in light of this disclosure using conventional chemistry.

In preparing compounds or a stereoisomer, atropisomer, tautomer, or pharmaceutically acceptable salt thereof as described herein protection of remote functionality (e.g., primary or secondary amine) of intermediates can be necessary. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz) and 9-fluorenylmethyleneoxycarbonyl (Fmoc). The need for such protection can be readily determined. For a general description of protecting groups and their use, see T. W. Greene, Protective Groups in Organic Synthesis, John Wiley & Sons, New York, 1991.

In the methods of preparing compounds or a stereoisomer, atropisomer, tautomer, or pharmaceutically acceptable salt thereof as described herein, it can be advantageous to separate reaction products from one another and/or from starting materials. The desired products of each step or series of steps is separated and/or purified to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like. Selection of appropriate methods of separation depends on the nature of the materials involved, such as, boiling point and molecular weight in distillation and sublimation, presence or absence of polar functional groups in chromatography, stability of materials in acidic and basic media in multiphase extraction, and the like.

Diastereomeric mixtures can be separated into their individual diastereomers on the basis of their physical chemical differences by methods such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Also, some of the compounds or a stereoisomer, atropisomer, tautomer, or pharmaceutically acceptable salt thereof as described herein described herein can be atropisomers (e.g., substituted biaryls). Enantiomers can also be separated by use of a chiral HPLC column.

A single stereoisomer, e.g., an enantiomer, substantially free of its stereoisomer can be obtained by resolution of the racemic mixture using a method such as formation of diastereomers using optically active resolving agents (Eliel, E. and Wilen, S. “Stereochemistry of Organic Compounds,” John Wiley & Sons, Inc., New York, 1994; Lochmuller, C. H., (1975) J. Chromatogr., 113(3):283-302). Racemic mixtures of chiral compounds or pharmaceutically acceptable salts thereof described herein can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: “Drug Stereochemistry, Analytical Methods and Pharmacology,” Irving W. Wainer, Ed., Marcel Dekker, Inc., New York (1993).

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts can be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (E. and Wilen, S. “Stereochemistry of Organic Compounds”, John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III. J. Org. Chem. (1982) 47:4165), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (“Chiral Liquid Chromatography” (1989) W. J. Lough, Ed., Chapman and Hall, New York; Okamoto, J. Chromatogr., (1990) 513:375-378). Enriched or purified enantiomers can be distinguished by methods used to distinguish other chiral molecules with asymmetric carbon atoms, such as optical rotation and circular dichroism.

The chemical reactions described herein may be readily adapted to prepare other compounds and pharmaceutically acceptable salts thereof described herein. For example, the synthesis of non-exemplified compounds and pharmaceutically acceptable salts thereof described herein may be successfully performed by modifications apparent to those skilled in the art, e.g., by appropriately protecting interfering groups, by utilizing other suitable reagents known in the art other than those described, or by making routine modifications of reaction conditions. Alternatively, other reactions disclosed herein or known in the art will be recognized as having applicability for preparing other compounds and pharmaceutically acceptable salts thereof described herein.

### PHARMACEUTICAL FORMULATIONS

Also provided herein are pharmaceutical compositions comprising compound or a stereoisomer, atropisomer, tautomer, or pharmaceutically acceptable salt thereof as described herein and one or more pharmaceutically acceptable excipients.

Compounds or a stereoisomer, atropisomer, tautomer, or pharmaceutically acceptable salt thereof as described herein as described herein can be formulated in accordance with standard pharmaceutical practice as a pharmaceutical composition. Thus, further provided herein is a pharmaceutical composition comprising a compound or a stereoisomer, atropisomer, tautomer, or pharmaceutically acceptable salt thereof as described herein as described herein and one or more pharmaceutically acceptable excipients.

A typical formulation is prepared by mixing a compound or pharmaceutically acceptable salt thereof as described herein and an excipient. Suitable carriers, diluents and excipients include, but are not limited to, materials such as carbohydrates, waxes, water soluble and/or swellable polymers, hydrophilic or hydrophobic materials, gelatin, oils, solvents, water and the like. The particular excipient used will depend upon the means and purpose for which the compound or pharmaceutically acceptable salt thereof as described herein is being applied. Solvents are generally selected based on solvents recognized as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations can also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound described herein or pharmaceutical composition thereof) or aid in the manufacturing of the pharmaceutical product (i.e., medicament).

The formulations can be prepared using conventional dissolution and mixing procedures. For example, the bulk drug substance (i.e., compound or pharmaceutically acceptable salt thereof as described herein or stabilized form thereof (e.g., complex with a cyclodextrin derivative or other known complexation agent) is dissolved in a suitable solvent in the presence of one or more of the excipients described above. The compound or a stereoisomer, atropisomer, tautomer, or pharmaceutically acceptable salt thereof as described herein as described herein is typically formulated into pharmaceutical dosage forms to provide an easily controllable dosage of the drug and to enable patient compliance with the prescribed regimen.

The pharmaceutical composition (or formulation) for application can be packaged in a variety of ways depending upon the method used for administering the drug. Generally, an article for distribution includes a container having deposited therein the pharmaceutical formulation in an appropriate form. Suitable containers include materials such as bottles (plastic and glass), sachets, ampoules, plastic bags, metal cylinders, and the like. The container can also include a tamper-proof assemblage to prevent indiscreet access to the contents of the package. In addition, the container has deposited thereon a label that describes the contents of the container. The label can also include appropriate warnings.

Pharmaceutical formulations of the compound or a stereoisomer, atropisomer, tautomer, or pharmaceutically acceptable salt thereof as described herein can be prepared for various routes and types of administration. For example, a compound or a stereoisomer, atropisomer, tautomer, or pharmaceutically acceptable salt thereof having the desired degree of purity can optionally be mixed with one or more pharmaceutically acceptable excipients (Remington's Pharmaceutical Sciences (1980) 16th edition, Osol, A. Ed.), in the form of a lyophilized formulation, milled powder, or an aqueous solution.

Formulation can be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but can range from about 3 to about 8. For example, formulation in an acetate buffer at pH 5 can be a suitable embodiment.

The pharmaceutical composition ordinarily can be stored as a solid composition, a lyophilized formulation or as an aqueous solution.

The pharmaceutical compositions described herein can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The effective amount of the compound or a stereoisomer, atropisomer, tautomer, or pharmaceutically acceptable salt thereof to be administered will be governed by such considerations, and is the minimum amount necessary to ameliorate, or treat the hyperproliferative disorder.

As a general proposition, the initial pharmaceutically effective amount of the compound or a stereoisomer, atropisomer, tautomer, or pharmaceutically acceptable salt thereof administered parenterally per dose will be in the range of about 0.01-100 mg/kg, namely about 0.1 to 20 mg/kg of patient body weight per day, with the typical initial range of compound used being 0.3 to 15 mg/kg/day. In another embodiment, a pharmaceutical composition described herein comprises an effective amount of a compound or a stereoisomer, atropisomer, tautomer, or pharmaceutically acceptable salt thereof described herein in an amount of about: 1 mg-10 mg; 10 mg-25 mg; 20 mg-50 mg; 50 mg-75 mg; 70 mg-100 mg; 100 mg-150 mg; 100 mg-200 mg; 100 mg-500 mg; 200 mg-500 mg; 250 mg-500 mg; 500 mg-1000 mg; or 750 mg-1000 mg.

Acceptable pharmaceutically acceptable excipients are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations of compounds or pharmaceutically acceptable salts thereof as described herein may be prepared. Suitable examples of sustained-release preparations include semipermeable matrices of solid hydrophobic polymers containing a compound or pharmaceutically acceptable salt thereof as described herein, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

The formulations include those suitable for the administration routes detailed herein. The formulations can conveniently be presented in unit dosage form and can be prepared by any methods. Techniques and formulations generally are found in Remington's Pharmaceutical Sciences (Mack Publishing Co., Easton, PA). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of a compound or a stereoisomer, atropisomer, tautomer, or pharmaceutically acceptable salt thereof as described herein suitable for oral administration can be prepared as discrete units such as pills, capsules, cachets or tablets each containing a predetermined amount of such compound or a stereoisomer, atropisomer, tautomer, or pharmaceutically acceptable salt thereof. Compressed tablets can be prepared by compressing in a suitable machine the active ingredient in a free-flowing form such as a powder or granules, optionally mixed with a binder, lubricant, inert diluent, preservative, surface active or dispersing agent. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom. Tablets, troches, lozenges, aqueous or oil suspensions, dispersible powders or granules, emulsions, hard or soft capsules, e.g., gelatin capsules, syrups or elixirs can be prepared for oral use. Formulations of compounds or pharmaceutically acceptable salts thereof as described herein intended for oral use can be prepared according to any method for the manufacture of pharmaceutical compositions and such compositions can contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients can be employed with either a paraffinic or a water-miscible ointment base. Alternatively, the active ingredients can be formulated in a cream with an oil-in-water cream base. If desired, the aqueous phase of the cream base can include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations can desirably include a compound which enhances absorption or penetration of the active ingredient through the skin or other affected areas. Examples of such dermal penetration enhancers include dimethyl sulfoxide and related analogs. The oily phase of the emulsions of compositions provided herein can be constituted from known ingredients in a known manner. While the phase can comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of described herein include Tween® 60, Span® 80, cetostearyl alcohol, benzyl alcohol, myristyl alcohol, glyceryl mono-stearate and sodium lauryl sulfate.

Aqueous suspensions comprising a compound or a stereoisomer, atropisomer, tautomer, or pharmaceutically acceptable salt thereof as described herein can contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose or saccharin.

The pharmaceutical compositions of a compound or a stereoisomer, atropisomer, tautomer, or pharmaceutically acceptable salt thereof as described herein can be in the form of a sterile injectable preparation, such as a sterile injectable aqueous or oleaginous suspension. This suspension can be formulated using suitable dispersing or wetting agents and suspending agents which have been mentioned above. The sterile injectable preparation can also be a sterile injectable solution or suspension in a non-toxic parenterally acceptable diluent or solvent, such as a solution in 1,3-butanediol or prepared as a lyophilized powder. Among the acceptable vehicles and solvents that can be employed are water, Ringer's solution and isotonic sodium chloride solution. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables.

The amount of active ingredient that can be combined with the carrier material to produce a single dosage form will vary depending upon the host treated and the particular mode of administration. For example, a time-release formulation intended for oral administration to humans can contain approximately 1 to 1000 mg of active material compounded with an appropriate and convenient amount of carrier material which can vary from about 5 to about 95% of the total compositions (weight:weight). The pharmaceutical composition can be prepared to provide easily measurable amounts for administration. For example, an aqueous solution intended for intravenous infusion can contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for Example 6about 0.5 to 10% w/w, for Example 6about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration can be presented as a suppository with a suitable base comprising for Example 6cocoa butter or a salicylate.

Formulations suitable for intrapulmonary or nasal administration have a particle size for Example 6in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration can be prepared according to conventional methods and can be delivered with other therapeutic agents such as compounds heretofore used in the treatment or prophylaxis disorders as described below.

Formulations suitable for vaginal administration can be presented as pessaries, tampons, creams, gels, pastes, foams or spray formulations containing in addition to the active ingredient such carriers considered to be appropriate.

The formulations can be packaged in unit-dose or multi-dose containers, for Example 6sealed ampoules and vials, and can be stored in a freeze-dried (lyophilized) condition requiring only the addition of the sterile liquid carrier, for Example 6water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In one embodiment, the compound or a stereoisomer, atropisomer, tautomer, or pharmaceutically acceptable salt thereof are formulated as a prodrug. The term prodrug as used herein refers to a derivative of a compound that can be hydrolyzed, oxidized, or cleaved under biological conditions to provide the compound or a stereoisomer, atropisomer, tautomer, or pharmaceutically acceptable salt thereof. A prodrug as defined herein includes derivatives comprising one or more moieties that modulate or improve one or more physical, physiological or pharmaceutical property such as, but not limited to, solubiliy, permeability, uptake, biodistribution, metabolic stability, onset of action or some other druglike property, and is transformed to the bioactive or more biologically active substance as provided herein. In one embodiment, a prodrug herein has no biological activity until release of the compound or pharmaceutically acceptable salt thereof.

### Methods of Administration

Compounds or a stereoisomer, atropisomer, tautomer, or pharmaceutically acceptable salt thereof described herein can be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal. For local immunosuppressive treatment, the compounds can be administered by intralesional administration, including perfusing or otherwise contacting the graft with the inhibitor before transplantation. It will be appreciated that the preferred route can vary with for Example 6the condition of the recipient. Where the compound or a stereoisomer, atropisomer, tautomer, or pharmaceutically acceptable salt thereof is administered orally, it can be formulated as a pill, capsule, tablet, etc. with a pharmaceutically acceptable carrier or excipient. Where the compound or a stereoisomer, atropisomer, tautomer, or pharmaceutically acceptable salt thereof is administered parenterally, it can be formulated with a pharmaceutically acceptable parenteral vehicle and in a unit dosage injectable form, as detailed below.

Thus, in one aspect provided herein is a pharmaceutical composition comprising a compound or pharmaceutically acceptable salt thereof as described herein and one or more pharmaceutically acceptable excipients. In one embodiment, compounds or a stereoisomer, atropisomer, tautomer, or pharmaceutically acceptable salt thereof described herein are administered as pharmaceutical compositions capable of being administered to a subject orally or parenterally. The compounds or a stereoisomer, atropisomer, tautomer, or pharmaceutically acceptable salt thereof described herein can be formulated for topical or parenteral use where the compound or a stereoisomer, atropisomer, tautomer, or pharmaceutically acceptable salt thereof is dissolved or otherwise suspended in a solution suitable for injections, suspensions, syrups, creams, ointments, gels, sprays, solutions and emulsions.

Oral administration can promote patient compliance in taking the compound (e.g. formulated as a pharmaceutical composition), thereby increasing compliance and efficacy. Oral pharmaceutical compositions comprising a compound described herein include, but are not limited to, tablets (e.g. coated, non-coated and chewable) and capsules (e.g. hard gelatin capsules, soft gelatin capsules, enteric coated capsules, and sustained release capsules). Tablets can be prepared by direct compression, by wet granulation, or by dry granulation. Oral pharmaceutical compositions comprising a compound described herein can be formulated for delayed or prolonged release.

A dose to treat human patients can range from about 10 mg to about 1000 mg of a compound described herein. A typical dose can be about 100 mg to about 300 mg of the compound. A dose can be administered once a day (QID), twice per day (BID), or more frequently, depending on the pharmacokinetic and pharmacodynamic properties, including absorption, distribution, metabolism, and excretion of the particular compound. Administration as used herein refers to the frequency of dosing and not, for example, the number of individual units a patient described herein must take for a dose. Thus, in some embodiments, a patient may take two or more dosage units (e.g. two or more pills/tablets/capsules) QD. In addition, toxicity factors can influence the dosage and administration regimen. When administered orally, the pill, capsule, or tablet can be ingested daily or less frequently for a specified period of time. The regimen can be repeated for a number of cycles of therapy.

### Methods of Treatment and Uses

The compounds or a stereoisomer, atropisomer, tautomer, or pharmaceutically acceptable salt thereof described herein are useful as Ras inhibitors. In one aspect, the compounds or a stereoisomer, atropisomer, tautomer, or pharmaceutically acceptable salt thereof described herein are useful as KRas inhibitors. In another embodiment, the compounds or a stereoisomer, atropisomer, tautomer, or pharmaceutically acceptable salt thereof described herein are useful as pan-KRas inhibitors (i.e. compounds that inhibit the activity of a mutant KRas protein). In one such embodiment, the compounds or a stereoisomer, atropisomer, tautomer, or pharmaceutically acceptable salt thereof described herein are useful as pan-KRas inhibitors comprising a mutation at the 12 position (e.g. a G12X mutation where X is a mutation of the naturally occurring Gly residue to different amino acid). In one embodiment, the compounds of Table 1 and Table 2 or a stereoisomer, atropisomer, tautomer, or pharmaceutically acceptable salt thereof described herein are useful as pan-KRas inhibitors inhibitors.

Provided herein are methods of contacting a cell, such as an ex vivo cell, with a compound or a stereoisomer, atropisomer, tautomer, or pharmaceutically acceptable salt thereof described herein, to inhibit KRas activity in the cell. In another embodiment, the activity is mutant KRas activity (e.g. mutant pan-KRas activity).

As used herein, inhibition of the activity of more than one KRas mutant is referred to as pan-KRas inhibition. In such instances, a compound or pharmaceutically acceptable salt thereof as described herein inhibits the activity of more than one mutant KRas protein. In certain instances, such compounds or pharmaceutically acceptable salts thereof selectively inhibit more than one mutant KRas protein relative to the wildtype (WT) KRas protein activity. In one such embodiment, a pan-KRas inhibitor as described herein and used in the methods provided herein inhibits at least one mutant KRas protein at least 5×, 8×, 10×, 12×, 15×, 20×, 24×, 27×, 50×, 100×, 500×, 700×, 1000×, 1300×, 1700×, 2000×, 5000×, or more greater than WT KRas protein. In one embodiment, such a KRas mutation is in the SWII domain. In one embodiment, such a KRas mutation corresponds to a change in the natural amino acid at the position corresponding to G12, G13, Q61, or A146. In such embodiments, such compounds are useful in the methods described herein where such cancer or disease is mediated by a KRasG12X mutation (e.g. where X is C, D, R, or V). In some embodiments, the mutation corresponds to G12A, G12C, G12D, G12R, G12S, G12V, G13A, G13C, G13D, G13R, G13S, G13V, Q61E, Q61H, Q61K, Q61L, Q61P, Q61R, A146T, A146P, A146V, or A146T.

Further provided herein are methods of treating a cancer comprising a KRas mutation, the method comprising administering to a patient having such cancer, an effective amount of a compound or a stereoisomer, atropisomer, tautomer, or pharmaceutically acceptable salt thereof or a pharmaceutical composition as described herein. In one embodiment, the mutation is a known KRas mutation as described herein (e.g. A KRasG12X mutation) and demonstrates pan-KRas inhibition.

In one embodiment, the methods further comprise testing a sample (e.g. as set forth herein) from the patient before administration of a compound of pharmaceutically acceptable salt thereof described herein for the absence or presence of a KRas mutation (e.g. A KRasG12X mutation). In one such embodiment, a compound or stereoisomer, atropisomer, tautomer, or pharmaceutically acceptable salt thereof or pharmaceutical composition described herein is administered to the patient after the patient sample is determined to be positive for (e.g. the presence of) a KRas mutation. In one embodiment, the methods further comprise testing a sample (e.g. as set forth herein) from the patient before administration of a compound of pharmaceutically acceptable salt thereof described herein for the absence or presence of a KRas mutation, wherein the compound or stereoisomer, atropisomer, tautomer, or pharmaceutically acceptable salt thereof or pharmaceutical composition described herein is administered to the patient after the patient sample is determined to be positive for (e.g. the presence of) such KRas mutation.

The methods of treating a cancer described herein relate to the treatment of cancer such as acute myeloid leukemia, cancer in adolescents, childhood adrenocortical carcinoma, AIDS-related cancers (e.g. lymphoma and Kaposi's sarcoma), anal cancer, appendix cancer, astrocytomas, atypical teratoid rhabdoid tumor, basal cell carcinoma, bile duct cancer, bladder cancer, bone cancer, brain stem glioma, brain tumor, breast cancer, bronchial tumors, Burkitt lymphoma, carcinoid tumor, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, Merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, oropharyngeal cancer, ovarian cancer, pancreatic cancer, papillomatosis, paraganglioma, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer.

In some embodiments, the cancer is a hematological cancer, pancreatic cancer, MYH associated polyposis, colorectal cancer or lung cancer. In one embodiment, the cancer is lung cancer, colorectal cancer, appendicial cancer, or pancreatic cancer. In one embodiment, the cancer is pancreatic cancer, lung cancer, or colon cancer. The lung cancer can be adenocarcinoma, non-small cell lung cancer (NSCLC), or small cell lung cancer (SCLC). In one embodiment, the cancer is colorectal cancer. In another embodiment, the cancer is pancreatic cancer. In one embodiment, the cancer is lung adenocarcinoma.

The methods provided herein can also comprise testing a sample from the patient before administration of a compound or stereoisomer, atropisomer, tautomer, or pharmaceutically acceptable salt thereof described herein for the absence or presence of a KRas mutation corresponding to the 12 position of KRas (e.g. Gly12). In one embodiment, a compound, stereoisomer, atropisomer, tautomer, or pharmaceutically acceptable salt thereof or pharmaceutical composition is administered to the patient after the patient sample shows the presence of a KRas mutation corresponding to the 12 position of KRas (e.g. Gly12). In one embodiment, a compound or stereoisomer, atropisomer, tautomer, or pharmaceutically acceptable salt thereof described herein is not administered unless a patient sample comprises a KRas mutation corresponding to the 12 position of KRas (e.g. Gly12).

The methods provided herein can further comprise testing a sample from the patient before administration of a compound or stereoisomer, atropisomer, tautomer, or pharmaceutically acceptable salt thereof described herein for the absence or presence of a KRas mutation, where the compound or stereoisomer, atropisomer, tautomer, or pharmaceutically acceptable salt thereof described herein has pan-KRas inhibition. In one embodiment, a compound, stereoisomer, atropisomer, tautomer, or pharmaceutically acceptable salt thereof or pharmaceutical composition is administered to the patient after the patient sample shows the presence of a KRas mutation, where the compound or stereoisomer, atropisomer, tautomer, or pharmaceutically acceptable salt thereof described herein has pan-KRas inhibition. In one embodiment, a compound or stereoisomer, atropisomer, tautomer, or pharmaceutically acceptable salt thereof described herein is not administered unless a patient sample comprises a KRas mutation, where the compound or stereoisomer, atropisomer, tautomer, or pharmaceutically acceptable salt thereof described herein has pan-KRas inhibition.

In one embodiment, the cancer is pancreatic cancer, lung cancer, or colorectal cancer. In another embodiment, the pancreatic cancer, lung cancer, or colorectal cancer comprises a KRasG12X mutation. In still another embodiment, the cancer is tissue agnostic but comprises a KRasG12X mutation.

In another embodiment, the pancreatic cancer, lung cancer, or colorectal cancer comprises a KRas mutation. In one such embodiment, the cancer is tissue agnostic but comprises a KRas mutation. In such embodiments, the cancer can be treated as described herein with a compound or stereoisomer, atropisomer, tautomer, or pharmaceutically acceptable salt thereof described herein having pan-KRas inhibition. In some embodiments, the KRas mutation is a mutation of a residue other than Gly12.

Further provided herein herein are methods of treating lung cancer comprising a KRasG12X mutation in a patient having such a lung cancer, the method comprising administering to the patient an effective amount of a compound or a stereoisomer, atropisomer, tautomer, or pharmaceutically acceptable salt thereof (or a pharmaceutical composition comprising the same) described herein to the patient. In one embodiment, the lung cancer is non-small cell lung carcinoma (NSCLC). The NSCLC can be, for example, adenocarcinoma, squamous-cell lung carcinoma or large-cell lung carcinoma. In another embodiment, the lung cancer is small cell lung carcinoma. In still another embodiment, the lung cancer is glandular tumors, carcinoid tumors or undifferentiated carcinomas. The lung cancer can be stage I or II lung cancer. In one embodiment, the lung cancer is stage III or IV lung cancer. The methods provided herein include administration of the compound as a 1 L therapy. In one embodiment, the compound or stereoisomer, atropisomer, tautomer, or pharmaceutically acceptable salt thereof described herein has pan-KRas inhibition.

Still further provided herein are methods of treating lung cancer comprising a KRas mutation (e.g. corresponding to position other than Gly12) in a patient having such a lung cancer, the method comprising administering to the patient an effective amount of a compound or a stereoisomer, atropisomer, tautomer, or pharmaceutically acceptable salt thereof (or a pharmaceutical composition comprising the same) described herein, where the compound or stereoisomer, atropisomer, tautomer, or pharmaceutically acceptable salt thereof described herein has pan-KRas inhibition, to the patient. In one embodiment, the lung cancer is non-small cell lung carcinoma (NSCLC). The NSCLC can be, for example, adenocarcinoma, squamous-cell lung carcinoma or large-cell lung carcinoma. In another embodiment, the lung cancer is small cell lung carcinoma. In still another embodiment, the lung cancer is glandular tumors, carcinoid tumors or undifferentiated carcinomas. The lung cancer can be stage I or II lung cancer. In one embodiment, the lung cancer is stage III or IV lung cancer. The methods provided herein include administration of the compound as a 1 L therapy.

Further provided herein are methods of treating pancreatic cancer comprising a KRasG12X mutation in a patient having such pancreatic cancer, the method comprising administering to the patient an effective amount of a compound or stereoisomer, atropisomer, tautomer, or pharmaceutically acceptable salt thereof described herein to the patient. In one embodiment, the patient has been previously treated with radiation and one or more chemotherapy agents. In one embodiment, the pancreatic cancer is stage 0, I, or II. In another embodiment, the pancreatic cancer is stage III or stage IV. In one embodiment, the compound or stereoisomer, atropisomer, tautomer, or pharmaceutically acceptable salt thereof described herein has pan-KRas inhibition.

Further provided herein are methods of treating pancreatic cancer comprising a KRas mutation (e.g. corresponding to position other than Gly12) in a patient having such pancreatic cancer, the method comprising administering to the patient an effective amount of a compound or stereoisomer, atropisomer, tautomer, or pharmaceutically acceptable salt thereof described herein, where the compound or stereoisomer, atropisomer, tautomer, or pharmaceutically acceptable salt thereof described herein has pan-KRas inhibition, to the patient. In one embodiment, the patient has been previously treated with radiation and one or more chemotherapy agents. In one embodiment, the pancreatic cancer is stage 0, I, or II. In another embodiment, the pancreatic cancer is stage III or stage IV.

Still further provided herein are methods of treating colon cancer comprising a KRasG12X mutation in a patient having such colon cancer, the method comprising administering to the patient an effective amount of a compound or stereoisomer, atropisomer, tautomer, or pharmaceutically acceptable salt thereof described herein to the patient. In one embodiment, the colon cancer is stage I or II. In another embodiment, the colon cancer is stage III or stage IV. In one embodiment, the compound or stereoisomer, atropisomer, tautomer, or pharmaceutically acceptable salt thereof described herein has pan-KRas inhibition.

Still further provided herein are methods of treating colon cancer comprising a KRas mutation (e.g. corresponding to position other than Gly12) in a patient having such colon cancer, the method comprising administering to the patient an effective amount of a compound or stereoisomer, atropisomer, tautomer, or pharmaceutically acceptable salt thereof described herein, where the compound or stereoisomer, atropisomer, tautomer, or pharmaceutically acceptable salt thereof described herein has pan-KRas inhibition, to the patient. In one embodiment, the colon cancer is stage I or II. In another embodiment, the colon cancer is stage III or stage IV.

Further provided herein are methods of treating tissue agnostic cancer comprising a KRasG12X mutation. In one embodiment of such methods, the method (Ag2) comprises:
determining the absence or presence of a KRasG12X mutation in a sample taken from a patient with a suspected diagnosed cancer; and
administering to the patient an effective amount of a compound or stereoisomer, atropisomer, tautomer, or pharmaceutically acceptable salt thereof described herein. In one embodiment, the compound or stereoisomer, atropisomer, tautomer, or pharmaceutically acceptable salt thereof described herein has pan-KRas inhibition.

Further provided herein are methods of treating tissue agnostic cancer comprising a KRas mutation (e.g. corresponding to position other than Gly12). In one embodiment of such methods, the method (Ag3) comprises:
determining the absence or presence of a KRas mutation in a sample taken from a patient with a suspected diagnosed cancer; and
administering to the patient an effective amount of a compound or stereoisomer, atropisomer, tautomer, or pharmaceutically acceptable salt thereof described herein, where the compound or stereoisomer, atropisomer, tautomer, or pharmaceutically acceptable salt thereof described herein has pan-KRas inhibition.

In one embodiment of the methods of Ag1, Ag2, and Ag3, the patient is diagnosed with a cancer described herein. In another embodiment of the methods of Ag1, Ag2, and Ag3, the sample is a tumor sample taken from the subject. In one such embodiment, the sample is taken before administration of any therapy. In another such embodiment, the sample is taken before administration of a compound or stereoisomer, atropisomer, tautomer, or pharmaceutically acceptable salt thereof described herein and after administration of another chemotherapeutic agent. In another embodiment of the methods of Ag1, Ag2, and Ag3, the compound or stereoisomer, atropisomer, tautomer, or pharmaceutically acceptable salt thereof described herein is administered as provided herein (e.g. orally).

Also provided herein is a compound or stereoisomer, atropisomer, tautomer, or pharmaceutically acceptable salt thereof for use as a therapeutically active substance. In another such embodiment, the compound or stereoisomer, atropisomer, tautomer, or pharmaceutically acceptable salt thereof can be for the therapeutic treatment of a cancer comprising a KRasG12X mutation. In one embodiment, the compound or stereoisomer, atropisomer, tautomer, or pharmaceutically acceptable salt thereof described herein has pan-KRas inhibition.

In still another such embodiment, the compound or stereoisomer, atropisomer, tautomer, or pharmaceutically acceptable salt thereof can be for the therapeutic treatment of a cancer comprising a KRas mutation (e.g. corresponding to position other than Gly12) where the compound or stereoisomer, atropisomer, tautomer, or pharmaceutically acceptable salt thereof described herein has pan-KRas inhibition.

Further provided herein a compound or stereoisomer, atropisomer, tautomer, or pharmaceutically acceptable salt thereof for the therapeutic and/or prophylactic treatment of a cancer comprising a KRasG12X mutation. In one embodiment, the compound or stereoisomer, atropisomer, tautomer, or pharmaceutically acceptable salt thereof described herein has pan-KRas inhibition.

Still further provided herein is a compound or stereoisomer, atropisomer, tautomer, or pharmaceutically acceptable salt thereof for the therapeutic and/or prophylactic treatment of a cancer comprising a KRas mutation (e.g. corresponding to position Gly12) where the compound or stereoisomer, atropisomer, tautomer, or pharmaceutically acceptable salt thereof described herein has pan-KRas inhibition.

In one embodiment, a compound or stereoisomer, atropisomer, tautomer, or pharmaceutically acceptable salt thereof described herein is used in the preparation of a medicament for the therapeutic treatment of a cancer comprising a KRasG12X. In one embodiment, the compound or stereoisomer, atropisomer, tautomer, or pharmaceutically acceptable salt thereof described herein has pan-KRas inhibition.

mutation. In one embodiment, a compound or stereoisomer, atropisomer, tautomer, or pharmaceutically acceptable salt thereof described herein is used in the preparation of a medicament for the therapeutic treatment of a cancer comprising a KRas mutation (e.g. corresponding to position other than Gly12) where the compound or stereoisomer, atropisomer, tautomer, or pharmaceutically acceptable salt thereof described herein has pan-KRas inhibition.

Still further provided herein are uses of a compound or stereoisomer, atropisomer, tautomer, or pharmaceutically acceptable salt thereof as described herein in the manufacture of a medicament for inhibiting tumor metastasis.

Further provided herein are methods for inhibiting tumor metastasis, the method comprising administering to a patient having a tumor a therapeutically effective amount of a compound or stereoisomer, atropisomer, tautomer, or pharmaceutically acceptable salt thereof described herein. In one embodiment, the inhibition is of a tumor comprising a KRasG12X mutation. In one embodiment, the compound or stereoisomer, atropisomer, tautomer, or pharmaceutically acceptable salt thereof described herein has pan-KRas inhibition.

In one embodiment, the inhibition is of a tumor comprising a KRas mutation (e.g. corresponding to position other than Gly12) where the compound or stereoisomer, atropisomer, tautomer, or pharmaceutically acceptable salt thereof described herein has pan-KRas inhibition.

In some embodiments of the methods described herein, inhibiting tumor metastasis in a patient described herein results in reduction of tumor size. In another embodiment, inhibiting tumor metastasis in a patient described herein results in stabilizing (e.g. no further growth) of tumor size. In another embodiment, inhibiting tumor metastasis in a patient described herein results in remission of the cancer and/or its symptoms.

Further provided herein are methods for inhibiting proliferation of a cell population, the method comprising contacting the cell population with a compound or stereoisomer, atropisomer, tautomer, or pharmaceutically acceptable salt thereof described herein. In one embodiment, the cell population is in a human patient. In another embodiment, the cell population comprises a KRasG12X mutation. In one embodiment, the compound or stereoisomer, atropisomer, tautomer, or pharmaceutically acceptable salt thereof described herein has pan-KRas inhibition.

In another embodiment, the cell population comprises a KRas mutation (e.g. corresponding to position other than Gly12) where the compound or stereoisomer, atropisomer, tautomer, or pharmaceutically acceptable salt thereof described herein has pan-KRas inhibition.

Further provided herein are methods of inhibiting KRas in a patient in need of therapy, comprising administering to the patient a therapeutically effective amount of a compound or stereoisomer, atropisomer, tautomer, or pharmaceutically acceptable salt thereof described herein. In one embodiment, the KRas inhibited is KRasG12X. In one embodiment, the compound or stereoisomer, atropisomer, tautomer, or pharmaceutically acceptable salt thereof described herein has pan-KRas inhibition.

In one embodiment, the KRas inhibited is a mutant KRas protein (e.g. corresponding to position other than Gly12) where the compound or stereoisomer, atropisomer, tautomer, or pharmaceutically acceptable salt thereof described herein has pan-KRas inhibition. In another embodiment, inhibiting KRas results in decreased tumor size. In another embodiment, inhibiting KRas results in remission of the cancer and/or its symptoms.

Further provided herein are methods for regulating activity of a KRas mutant protein, the method comprising reacting the mutant protein with a compound or stereoisomer, atropisomer, tautomer, or pharmaceutically acceptable salt thereof described herein. In one embodiment, the mutant protein comprises a KRasG12X mutation. In one embodiment, the compound or stereoisomer, atropisomer, tautomer, or pharmaceutically acceptable salt thereof described herein has pan-KRas inhibition.

In one embodiment, the mutant protein comprises a KRas mutation where the compound or stereoisomer, atropisomer, tautomer, or pharmaceutically acceptable salt thereof described herein has pan-KRas inhibition. In one embodiment, the activity of KRas is decreased after contacting with a compound or stereoisomer, atropisomer, tautomer, or pharmaceutically acceptable salt thereof described herein. In another embodiment, the downregulation of activity of the KRas mutant protein treats a cancer described herein in a patient described herein. In another embodiment, the downregulation of activity of the KRas mutant protein results in decreased tumor size. In another embodiment, the downregulation of activity of the KRas mutant protein results in remission of a cancer described herein and/or its symptoms.

In some embodiments, the methods provided herein comprise inhibiting KRasG12X activity in a cell by contacting said cell with an amount of a compound or stereoisomer, atropisomer, tautomer, or pharmaceutically acceptable salt thereof described herein sufficient to inhibit the activity of KRasG12X in said cell. In one embodiment, the compound or stereoisomer, atropisomer, tautomer, or pharmaceutically acceptable salt thereof described herein has pan-KRas inhibition.

In some embodiments, the methods provided herein comprise inhibiting KRasG12X activity in a tissue by contacting said tissue with an amount of a compound or stereoisomer, atropisomer, tautomer, or pharmaceutically acceptable salt thereof described herein sufficient to inhibit the activity of KRasG12X in said tissue. In some embodiments, the methods provided herein comprise inhibiting KRasG12X activity in a patient described herein by contacting said patient with an amount of a compound or stereoisomer, atropisomer, tautomer, or pharmaceutically acceptable salt thereof described herein sufficient to inhibit the activity of KRasG12X in said patient. In one embodiment, the compound or stereoisomer, atropisomer, tautomer, or pharmaceutically acceptable salt thereof described herein has pan-KRas inhibition.

In some embodiments, the methods provided herein comprise inhibiting mutant KRasG12X activity in a cell by contacting said cell with an amount of a compound or stereoisomer, atropisomer, tautomer, or pharmaceutically acceptable salt thereof described herein sufficient to inhibit the activity of mutant KRasG12X in said cell. In some embodiments, the methods provided herein comprise inhibiting mutant KRasG12X activity in a tissue by contacting said tissue with an amount of a compound or stereoisomer, atropisomer, tautomer, or pharmaceutically acceptable salt thereof described herein sufficient to inhibit the activity of mutant KRasG12X in said tissue. In some embodiments, the methods provided herein comprise inhibiting mutant KRasG12X activity in a patient described herein by contacting said patient with an amount of a compound or stereoisomer, atropisomer, tautomer, or pharmaceutically acceptable salt thereof described herein sufficient to inhibit the activity of mutant KRasG12X in said patient. In such embodiments, it is understood that the compound or stereoisomer, atropisomer, tautomer, or pharmaceutically acceptable salt thereof described herein has pan-KRas inhibition.

Further provided herein are methods for preparing a labeled KRasG12X mutant protein, the method comprising reacting a KRasG12X mutant protein with a labeled compound or stereoisomer, atropisomer, tautomer, or pharmaceutically acceptable salt thereof described herein to result in the labeled KRasG12X mutant protein. In one embodiment, the label is an imaging agent. In one embodiment, the labeled KRasG12X can be used to detect the absence or presence of KRasG12X mutant protein in a patient sample, thereby detecting the presence or absence of a cancer mediated by mutant KRas.

Further provided herein are methods for preparing a labeled mutant KRasG12X protein, the method comprising reacting a mutant KRasG12X protein with a labeled compound or stereoisomer, atropisomer, tautomer, or pharmaceutically acceptable salt thereof described herein, where the compound or stereoisomer, atropisomer, tautomer, or pharmaceutically acceptable salt thereof described herein has pan-KRas inhibition, to result in the labeled mutant KRasG12X protein. In one embodiment, the label is an imaging agent. In one embodiment, the labeled mutant KRasG12X protein can be used to detect the absence or presence of mutant KRasG12X protein in a patient sample, thereby detecting the presence or absence of a cancer mediated by mutant KRas.

Still further provided herein are methods of inhibiting Ras-mediated cell signaling. In one embodiment, the methods comprise contacting a cell with an effective amount of one or more compounds or a stereoisomer, atropisomer, tautomer, or pharmaceutically acceptable salt thereof disclosed herein thereof. Inhibition of Ras-mediated signal transduction can be assessed and demonstrated by a wide variety of ways known in the art. Non-limiting examples include a showing of (a) a decrease in GTPase activity of Ras; (b) a decrease in GTP binding affinity or an increase in GDP binding affinity; (c) an increase in K off of GTP or a decrease in K off of GDP; (d) a decrease in the levels of signaling transduction molecules downstream in the Ras pathway, such as a decrease in pMEK level; and/or (e) a decrease in binding of Ras complex to downstream signaling molecules including but not limited to Raf. Kits and commercially available assays can be utilized for determining one or more of the above.

KRas mutations have also been identified in hematological malignancies (e.g., cancers that affect blood, bone marrow, and/or lymph nodes). Accordingly, certain embodiments are directed to administration of a disclosed compound or stereoisomer, atropisomer, tautomer, or pharmaceutically acceptable salt thereof (e.g., in the form of a pharmaceutical composition) as described herein to a patient in need of treatment of a hematological malignancy. Such malignancies include, but are not limited to leukemias and lymphomas. For example, the presently disclosed compounds can be used for treatment of diseases such as acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias. In other embodiments, the compounds or a pharmaceutically acceptable salt thereof described herein are useful for treatment of lymphomas such as all subtypes of Hodgkin's lymphoma or non-Hodgkin's lymphoma.

Determining whether a tumor or cancer comprises a KRas mutation as described here can be undertaken by assessing the nucleotide sequence encoding the KRas protein, by assessing the amino acid sequence of the KRas protein, or by assessing the characteristics of a putative KRas mutant protein. The sequence of wild-type human KRas (e.g. Accession No. NP203524) is known in the art.

Methods for detecting a mutation in a KRas nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for KRas mutations described herein by real-time PCR. In real-time PCR, fluorescent probes specific for the KRas mutation are used. When a mutation is present, the probe binds and fluorescence is detected. In some embodiments, the KRas mutation is identified using a direct sequencing method of specific regions (e.g., exon 2 and/or exon 3) in the KRas gene. This technique will identify all possible mutations in the region sequenced.

Methods for determining whether a tumor or cancer comprises a KRas mutation described herein can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA.

Further provided herein are uses of a compound or stereoisomer, atropisomer, tautomer, or pharmaceutically acceptable salt thereof described herein, in the manufacture of a medicament for treating cancer. In some embodiments, the medicament is formulated for oral administration. In some embodiments, the medicament is formulated for injection. In some embodiments, the cancer comprises a KRasG12X mutation. In some embodiments, the cancer comprises a KRas mutation (e.g. mutation other than Gly12) where the compound or stereoisomer, atropisomer, tautomer, or pharmaceutically acceptable salt thereof described herein has pan-KRas inhibition. In some embodiments, the cancer is a hematological cancer, pancreatic cancer, MYH associated polyposis, colorectal cancer or lung cancer. In one embodiment, the cancer is lung cancer, colorectal cancer, or pancreatic cancer. In one embodiment, the cancer is colorectal cancer. In another embodiment, the cancer is pancreatic cancer. In some embodiments, the cancer is lung adenocarcinoma. In some embodiments, are uses of a compound or stereoisomer, atropisomer, tautomer, or pharmaceutically acceptable salt thereof described herein, in the manufacture of a medicament for inhibiting tumor metastasis.

Further provided herein is a compound or a pharmaceutically acceptable salt thereof described herein, for use in a method of treating cancer. In one embodiment, the cancer comprises a KRasG12X mutation. In one embodiment, the cancer comprises a KRas mutation (e.g. mutation other than Gly12) where the compound or stereoisomer, atropisomer, tautomer, or pharmaceutically acceptable salt thereof described herein has pan-KRas inhibition In one such embodiment, the cancer is a hematological cancer, pancreatic cancer, MYH associated polyposis, colorectal cancer or lung cancer. In one such embodiment, the cancer is lung cancer, colorectal cancer, or pancreatic cancer. In one such embodiment, the cancer is colorectal cancer. In one such embodiment, the cancer is pancreatic cancer. In one such embodiment, the cancer is lung adenocarcinoma.

### Combination Therapies

The compounds or a stereoisomer, atropisomer, tautomer, or pharmaceutically acceptable salt thereof described herein may be employed alone or in combination with other therapeutic agents for the treatment of a disease or disorder described herein. The second compound of the pharmaceutical combination formulation or dosing regimen preferably has complementary activities to the compound or a pharmaceutically acceptable salt thereof described herein such that they do not adversely affect each other. The combination therapy may provide “synergy” and prove “synergistic”, i.e., the effect achieved when the active ingredients used together is greater than the sum of the effects that results from using the compounds separately.

The combination therapy may be administered as a simultaneous or sequential regimen. When administered sequentially, the combination may be administered in two or more administrations. The combined administration includes co-administration, using separate formulations or a single pharmaceutical formulation, and consecutive administration in either order, wherein preferably there is a time period while both (or all) active agents simultaneously exert their biological activities.

Combination therapies herein comprise the administration of a compound or stereoisomer, atropisomer, tautomer, or pharmaceutically acceptable salt thereof described herein, and the use of at least one other treatment method. The amounts of the compound or stereoisomer, atropisomer, tautomer, or pharmaceutically acceptable salt thereof described herein and the other pharmaceutically active agent(s) and the relative timings of administration will be selected in order to achieve the desired combined therapeutic effect.

In various embodiments of the method, the additional therapeutic agent is an epidermal growth factor receptor (EGFR) inhibitor, phosphatidylinositol kinase (PI3K) inhibitor, insulin-like growth factor receptor (IGF1R) inhibitor, a Janus kinase (JAK) inhibitor, a Met kinase inhibitor, a SRC family kinase inhibitor, a mitogen-activated protein kinase (MEK) inhibitor, an extracellular-signal-regulated kinase (ERK) inhibitor, a topoisomerase inhibitor (such as irinotecan, or such as etoposide, or such as doxorubicin), a taxane (such as anti-microtubule agents including paclitaxel and docetaxel), an anti-metabolite agent (such as 5-FU or such as gemcitabine), or an alkylating agent (such as cisplatin or such as cyclophosphamide), or a taxane.

In some embodiments, the additional therapeutic agent is an epidermal growth factor receptor (EGFR) inhibitor, such as Erlotinib or such as Afatinib. In some embodiments the additional therapeutic agent is gefitinib, osimertinib, or dacomitinib. In some embodiments the additional therapeutic agent is a monoclonal antibody such as cetuximab (Erbitux) or panitumumab (Vectibix). In some embodiments the EGFR inhibitor is a dual or pan-HER inhibitor. In other embodiments, the additional therapeutic agent is a phosphatidylinositol-3-kinase (PI3K) inhibitor, such as GDC-0077, GDC-0941, MLN1117, BYL719 (Alpelisib) or BKM120 (Buparlisib). GDC-0941 refers to 2-(1H-indazol-4-yl)-6-(4-methanesulfonyl-piperazin-1-ylmethyl)-4-morpholin-4-yl-thieno[3,2-d]pyrimidine or a salt thereof (e.g., bismesylate salt).

In still other embodiments, the additional therapeutic agent is an insulin-like growth factor receptor (IGF1R) inhibitor. For example, in some embodiments the insulin-like growth factor receptor (IGF1R) inhibitor is NVP-AEW541. In other embodiments, the additional therapeutic agent is IGOSI-906 (Linsitinib), BMS-754807, or in other embodiments the additional therapeutic agent is a neutralizing monoclonal antibody specific to IGF1R such as AMG-479 (ganitumab), CP-751,871 (figitumumab), IMC-A12 (cixutumumab), MK-0646 (dalotuzumab), or R-1507 (robatumumab).

In some embodiments, the additional therapeutic agent is a SH2 containing protein tyrosine phosphatase-2 (SHP2) inhibitor. In one embodiment, the SHP2 inhibitor is RMC4550, GDC-1971, JAB-3068, JAB-3312, or TNO155, or a pharmaceutically acceptable salt thereof or combination thereof.

In some other embodiments, the additional therapeutic agent is a Janus kinase (JAK) inhibitor. In some embodiments, the additional therapeutic agent is CYT387, GLPG0634, Baricitinib, Lestaurtinib, momelotinib, Pacritinib, Ruxolitinib, or TG101348.

In some other embodiments, the additional therapeutic agent is an anti-glypican 3 antibody. In some embodiments, the anti-glypican 3 antibody is codrituzumab.

In some other embodiments, the additional therapeutic agent is an antibody drug conjugate (ADC). In some embodiments, the ADC is polatuzumab vedotin, RG7986, RG7882, RG6109, or RO7172369.

In some other embodiments, the additional therapeutic agent is an MDM2 antagonist. In some embodiments, the MDM2 antagonist is idasanutlin.

In some other embodiments, the additional therapeutic agent is an agonistic antibody against CD40. In some embodiments, the agonistic antibody against CD40 is selicrelumab (RG7876).

In some other embodiments, the additional therapeutic agent is a bispecific antibody. In some embodiments, the bispecific antibody is RG7828 (BTCT4465A), RG7802, RG7386 (FAP-DR5), RG6160, RG6026, ERY974, or anti-HER2/CD3.

In some other embodiments, the additional therapeutic agent is a targeted immunocytokine. In some embodiments, the targeted immunocytokine is RG7813 or RG7461.

In some other embodiments, the additional therapeutic agent is an antibody targeting colony stimulating factor-1 receptor (CSF-1R). In some embodiments, the CSF-1R antibody is emactuzumab.

In some other embodiments, the additional therapeutic agent is a personalised cancer vaccine. In some embodiments, the personalised cancer vaccine is RG6180.

In some other embodiments, the additional therapeutic agent is an inhibitor of BET (bromodomain and extraterminal family) proteins (BRD2/3/4/T). In some embodiments, the BET inhibitor is RG6146.

In some other embodiments, the additional therapeutic agent is an antibody designed to bind to TIGIT. In some embodiments, the anti-TIGIT antibody is RG6058 (MTIG7192A).

In some other embodiments, the additional therapeutic agent is a selective estrogen receptor degrader (SERD). In some other embodiments, the SERD is RG6047 (GDC-0927) or RG6171 (GDC-9545).

In some other embodiments the additional therapeutic agent is an MET kinase inhibitor, such as Crizotinib, tivantinib, AMG337, cabozantinib, or foretinib. In other embodiments the additional therapeutic agent is a neutralizing monoclonal antibody to MET such as onartuzumab.

In more embodiments, the additional therapeutic agent is a SRC family non-receptor tyrosine kinase inhibitor. For example, in some embodiments the additional therapeutic agent is an inhibitor of the subfamily of SRC family non-receptor tyrosine kinases. Exemplary inhibitors in this respect include Dasatinib. Other examples in this regard include Ponatinib, saracatinib, and bosutinib.

In yet other embodiments, the additional therapeutic agent is a mitogen-activated protein kinase (MEK) inhibitor. In some of these embodiments, the mitogen-activated protein kinase (MEK) inhibitor is trametinib, selumetinib, COTELLIC® (cobimetinib), PD0325901, or RO5126766. In other embodiments the MEK inhibitor is GSK-1120212, also known as trametinib.

In yet other embodiments, the additional therapeutic agent is an extracellular-signal-regulated kinase (ERK) inhibitor. In some of these embodiments, the mitogen-activated protein kinase (MEK) inhibitor is SCH722984 or GDC-0994.

In other embodiments the protein kinase inhibitor is taselisib, ipatasertib, GDC-0575, GDC-5573 (HM95573), RG6114 (GDC-0077), CK127, Afatinib, Axitinib, Atezolizumab, Bevacizumab, Bostutinib, Cetuximab, Crizotinib, Dasatinib, Erlotinib, Fostamatinib, Gefitinib, Imatinib, Lapatinib, Lenvatinib, Ibrutinib, Nilotinib, Panitumumab, Pazopanib, Pegaptanib, Ranibizumab, Ruxolitinib, Sorafenib, Sunitinib, SU6656, Trastuzumab, Tofacitinib, Vandetanib, or Vemurafenib. In still more embodiments, the additional therapeutic agent is a topoisomerase inhibitor. In some of these embodiments, the topoisomerase inhibitor is Irinotecan. In some more embodiments, the additional therapeutic agent is a taxane. Exemplary taxanes include Taxol and Docetaxel.

In addition to the above additional therapeutic agent, other chemotherapeutics are presently known in the art and can be used in combination with the compounds and pharmaceutically acceptable salts thereof described herein. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methyl melamines including altretamine, triethylenemelamine, triethylenephosphoramide, triethylenethiophosphaoramide and trimethylol melamine; nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™ chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide K; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; Xeloda®; ibandronate; camptothecin-11 (CPT-11); topoisomerase inhibitor RFS 2000; and difluoromethylornithine (DMFO). Where desired, the compounds or pharmaceutical acceptable salts thereof or pharmaceutical composition as described herein can be used in combination with commonly prescribed anti-cancer drugs such as Herceptin®, Avastin®, Gazyva®, Tecentriq®, Alecensa®, Perjeta®, Venclexta™, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

The exact method for administering the compound and the additional therapeutic agent will be apparent to one of ordinary skill in the art. In some exemplary embodiments the compound and the additional therapeutic agent are co-administered. In other embodiments, the compound and the additional therapeutic agent are separately administered.

In some embodiments, the compound and the additional therapeutic agent are administered with the second agent simultaneously or separately. This administration in combination can include simultaneous administration of the two agents in the same dosage form, simultaneous administration in separate dosage forms, and separate administration. That is, the compound and any of the additional therapeutic agents described herein can be formulated together in the same dosage form and administered simultaneously. Alternatively, the compound and any of the additional therapeutic agents described herein can be simultaneously administered, wherein both the agents are present in separate formulations. In another alternative, the compound can be administered just followed by any of the additional therapeutic agents described herein, or vice versa. In some embodiments of the separate administration protocol, the compound and any of the additional therapeutic agents described herein are administered a few minutes apart, or a few hours apart, or a few days apart.

### Articles of Manufacture

Also provided herein are articles of manufacture, or “kit”, containing materials useful for the treatment of a cancer provided herein. In one embodiment, the kit comprises a container comprising compound or stereoisomer, atropisomer, tautomer, or pharmaceutically acceptable salt thereof described herein. The kit may further comprise a label or package insert on or associated with the container. Suitable containers include, for example, bottles, vials, syringes, blister pack, etc. The container may be formed from a variety of materials such as glass or plastic. The container may hold a compound or a pharmaceutically acceptable salt thereof described herein or a formulation thereof which is effective for treating the condition and may have a sterile access port (for example, the container may be an intravenous solution bag or a vial having a stopper pierceable by a hypodermic injection needle). At least one active agent in the composition is a compound or a pharmaceutically acceptable salt thereof described herein. Alternatively, or additionally, the article of manufacture may further comprise a second container comprising a pharmaceutical diluent, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

In another embodiment, the kits are suitable for the delivery of solid oral forms of a compound or a pharmaceutically acceptable salt thereof described herein, such as tablets or capsules. Such a kit can include a number of unit dosages. An example of such a kit is a “blister pack”. Blister packs are well known in the packaging industry and are widely used for packaging pharmaceutical unit dosage forms.

### Embodiments

Provided below are exemplary embodiments of the invention described herein.

Embodiment 1: A compound or an atropisomer, stereoisomer, tautomer, or pharmaceutically acceptable salt thereof, having formula (I) as described herein, wherein;
Ring A is R⁸-substituted or unsubstituted 3-10 membered heterocycle, or R⁸-substituted or unsubstituted 5-10 membered heteroaryl;
R⁸ is independently halogen, CN, NH₂, NHC₁₋₃alkyl, R^{8A}-substituted or unsubstituted C₁₋₆alkyl, or R^{8A}-substituted or unsubstituted C₁₋₆haloalkyl;
R^{8A} is halogen, unsubstituted C₁₋₃alkyl, or unsubstituted C₁₋₃haloalkyl;
n is 1 or 2
R¹ is R⁷-substituted or unsubstituted indolyl, R⁷-substituted or unsubstituted benzofuranyl, R⁷-substituted or unsubstituted naphthyl, R⁷-substituted or unsubstituted indazolyl, R⁷-substituted or unsubstituted indenyl, R⁷-substituted or unsubstituted benzothiazolyl, R⁷-substituted or unsubstituted isoquinolinyl, R^{7A}-substituted or unsubstituted phenyl, or R^{7A}-substituted or unsubstituted pyridinyl;
each R⁷ is independently hydrogen, halogen, CN, CH₂OH, —OH, NH₂, N(Me)₂, unsubstituted C₁₋₃alkyl, unsubstituted C₂₋₅ alkynyl, unsubstituted C₁₋₃ haloalkyl, or unsubstituted cyclopropyl;
each R^{7a} is independently hydrogen, halogen, NH₂, N(Me)₂, unsubstituted C₁₋₃ alkyl, unsubstituted C₁₋₃haloalkyl, or unsubstituted cyclopropyl;
R² is OH, unsubstituted C₁₋₃alkoxy, unsubstituted C₁₋₃alkyl, or unsubstituted C₁₋₃haloalkyl;
R³ is R⁹-substituted or unsubstituted 4-10 membered heterocycle comprising N, S, or O;
R⁹ is independently halogen, oxo, unsubstituted C₁₋₃ alkyl, unsubstituted C₁₋₃ haloalkyl, unsubstituted C₁₋₃ alkoxy, R¹⁰-substituted or unsubstituted C₁₋₃ alkylidene, or
R¹⁰-substituted or unsubstituted C₃₋₄ cycloalkyl, or R¹⁰-substituted or unsubstituted 3 or 4-membered heterocycle; or wherein two R⁹ together form a C₃₋₅ cycloalkyl or 3-5 membered heterocycle;
R¹⁰ is hydrogen or halogen;
R⁴ is hydrogen, halogen, unsubstituted C₁₋₃alkyl, unsubstituted C₁₋₃alkoxy, or unsubstituted C₁₋₃haloalkyl; and
R⁵ and R⁶ are each independently hydrogen or unsubstituted C₁₋₃alkyl.

Embodiment 2: The compound of claim 1, wherein R¹ is R⁷-substituted or unsubstituted indolyl, R⁷-substituted or unsubstituted benzofuranyl, R⁷-substituted or unsubstituted naphthyl, R⁷-substituted or unsubstituted indazolyl, R⁷-substituted or unsubstituted indenyl, R⁷-substituted or unsubstituted benzothiazolyl, or R⁷-substituted or unsubstituted isoquinolinyl.

Embodiment 3: The compound of claim 1 or 2, wherein R¹ is R⁷-substituted or unsubstituted naphthyl or R⁷-substituted or unsubstituted isoquinolinyl.

Embodiment 4: The compound of any one of embodiments 1 to 3, wherein R¹ is a moiety of formula (C), (C₁), (C₂), or (C₃) as described herein.

Embodiment 5: The compound of any one of embodiments 1 to 4, wherein R¹ is

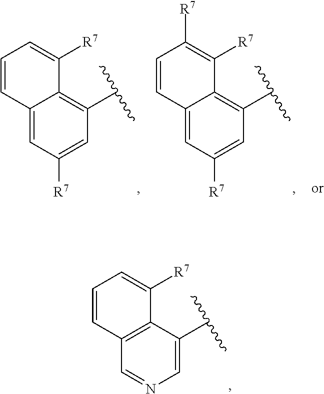

Embodiment 6: The compound of any one of embodiments 1 to 5 wherein R¹ is

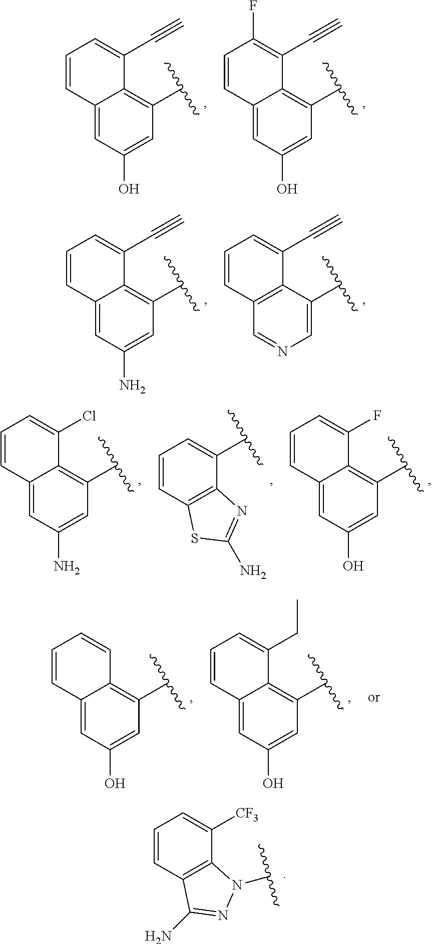

Embodiment 7: The compound of embodiment 1, wherein R¹ is R^{7A}-substituted or unsubstituted phenyl, or R^{7A}-substituted or unsubstituted or pyridinyl.

Embodiment 8: The compound of embodiments 1 or 7, wherein R¹ is a moiety of formula (A) or (B) as described herein wherein X¹ is N, CH, CF, or CCl; and R^{7A} is hydrogen, halogen, unsubstituted C₁₋₃ alkyl, or unsubstituted C₁₋₃ haloalkyl.

Embodiment 9: The compound of any one of embodiments 1 or 7-8, wherein R¹ is a moiety of formula (A) as described herein, wherein, X¹ is N, CH, CF, or CCl; and R^{7A} is hydrogen, halogen, unsubstituted C₁₋₃ alkyl, or unsubstituted C₁₋₃ haloalkyl.

Embodiment 10: The compound of embodiment 9, wherein X¹ is N.

Embodiment 11: The compound of any one of embodiments 1 or 9-10, wherein R¹ is

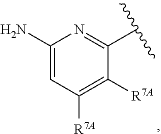

wherein R^{7A} is hydrogen, halogen, unsubstituted C₁₋₃ alkyl or unsubstituted C₁₋₃ haloalkyl.

Embodiment 12: The compound of any one of embodiments 1 or 9-11, R¹ is

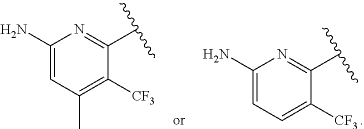 [SMILES: *c1nc(N)cc(C)c1C(F)(F)F ; *c1nc(N)ccc1C(F)(F)F]

Embodiment 13: The compound of embodiment 9, wherein X¹ is CH, CF, or CCl.

Embodiment 14: The compound of embodiments 9 or 13, wherein R¹ is

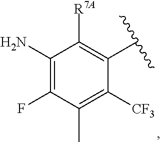

wherein R^{7A} is hydrogen, F, or Cl.

Embodiment 15: The compound of embodiments 9 or 13, wherein R¹ is

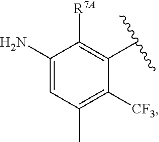

wherein R^{7A} is hydrogen, F, or Cl.

Embodiment 16: The compound of any one of embodiments 1 or 7-8, wherein R¹ is

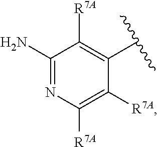

wherein R^{7A} is hydrogen, halogen, unsubstituted G1-3 alkyl, or R unsubstituted C₁₋₃ haloalkyl.

Embodiment 17: The compound of any one of embodiments 1 or 7-16, wherein R¹ is

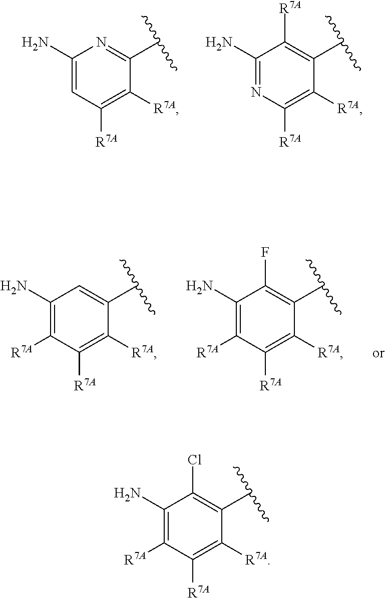

Embodiment 18: The compound of any one of embodiments 1 or 7-17, wherein R¹ is

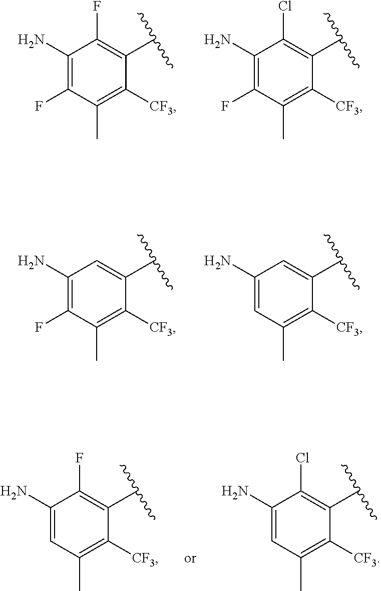 [SMILES: *c1c(F)c(N)c(F)c(C)c1C(F)(F)F ; *c1c(Cl)c(N)c(F)c(C)c1C(F)(F)F ; *c1cc(N)c(F)c(C)c1C(F)(F)F ; *c1cc(N)cc(C)c1C(F)(F)F ; *c1c(F)c(N)cc(C)c1C(F)(F)F ; *c1c(Cl)c(N)cc(C)c1C(F)(F)F]

Embodiment 19: The compound of any one of embodiments 1 or 7-18, R¹ is

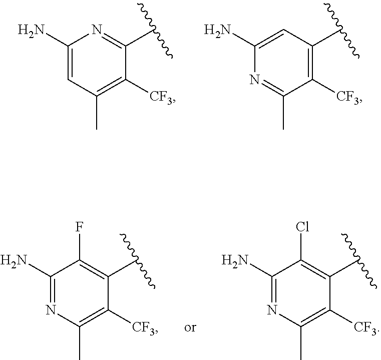 [SMILES: *c1nc(N)cc(C)c1C(F)(F)F ; *c1cc(N)nc(C)c1C(F)(F)F ; *c1c(F)c(N)nc(C)c1C(F)(F)F ; *c1c(Cl)c(N)nc(C)c1C(F)(F)F]

Embodiment 20: The compound of any one of embodiments 1 to 19, wherein R⁴ is Cl or F.

Embodiment 21: The compound of any one of embodiments 1 to 20, wherein R⁴ is F.

Embodiment 22: The compound of any one of embodiments 1 to 21, wherein R⁴ is Cl.

Embodiment 23: The compound of any one of embodiments 1 to 22, wherein R⁴ is OCH₃.

Embodiment 24: The compound of any one of embodiments 1 to 23, wherein R³ is a moiety of formula (DO) as described herein, wherein Z is C(R⁹)₂ or O; R⁹ is hydrogen, halogen or R¹⁰-substituted or unsubstituted C₁₋₃ alkylidene; or wherein two R⁹ together form a C₃₋₅ cycloalkyl or 3-5 membered heterocycle r is an integer of 0-12; j is 1, 2, or 3; and k is 1 or 2.

Embodiment 25: The compound of any one of embodiments 1 to 24, R³ is a moiety of formula (D) as described herein wherein R⁹ is halogen, —OCF₃, —OCHF₂, —OCH₂F, R¹⁰-substituted or unsubstituted C₁₋₃ alkylidene, or two R⁹ together form a R¹⁰-substituted or unsubstituted C₃₋₅ cycloalkyl; r is an integer of 0-12; j is 1, 2, or 3; and k is 1 or 2.

Embodiment 26: The compound of any one of embodiments 1 to 25, R³ is a moiety of formula (D1), (D2), (D3), or (D4) as described herein, each R⁹ is independently halogen or R¹⁰-substituted or unsubstituted C₁₋₃ alkylidene; each R¹⁰ is independently hydrogen or halogen; r is 0, 1, or 2; and s is 0, 1, or 2.

Embodiment 27: The compound of any one of embodiments 1 to 26, R³ is a moiety of formula (D1), (D2), or (D3) as described herein, wherein each R⁹ is independently halogen or R¹⁰-substituted or unsubstituted C₁₋₃ alkylidene; each R¹⁰ is independently hydrogen or halogen; r is 0, 1, or 2; and s is 0, 1, or 2.

Embodiment 28: The compound of any one of embodiments 1 to 27, wherein R³ is

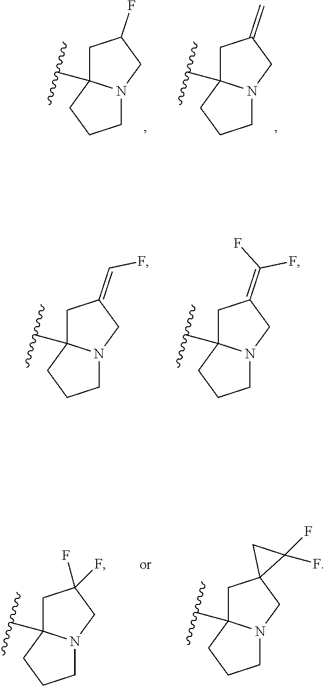

Embodiment 29: The compound of any one of embodiments 1 to 27, R³ is a moiety of formula(D1), where r is 1.

Embodiment 30: The compound of any one of embodiments 1 to 27, R³ is a moiety of formula(D2) where r is 0 and each R¹⁰ is independently hydrogen or F.

Embodiment 31: The compound of any one of embodiments 1 to 27, R³ is a moiety of formula (D3) where r is 0 and each R⁹ is independently hydrogen or halogen.

Embodiment 32: The compound of any one of embodiments 1 to 26, R³ is a moiety of formula (D4), wherein R¹⁰ is halogen and s is 0, 1, or 2.

Embodiment 33: The compound of any one of embodiments 1 to 25, R³ is a moiety of formula (D5), wherein R¹⁰ is halogen and s is 0, 1, or 2.

Embodiment 34: The compound of any one of embodiments 1 to 25, wherein R³ is a moiety of formula (E), wherein, each R⁹ is independently halogen, oxo, or unsubstituted C₁₋₃ alkyl; and r is 0, 1, or 2.

Embodiment 35: The compound of any one of embodiments 1 to 25, wherein R³ is a moiety of formula (F), wherein, each R⁹ is independently halogen, oxo, or unsubstituted C₁₋₃ alkyl; and r is 0, 1, or 2.

Embodiment 36: The compound of any one of embodiments 1 to 23, wherein R³ is moiety of formula (G) as described herein, wherein, each R⁹ is independently halogen, oxo, or unsubstituted C₁₋₃ alkyl; or wherein two R⁹ together form a C₃₋₅ cycloalkyl or 3-5 membered heterocycle; and r is 1 or 2.

Embodiment 37: The compound of any one of embodiments 1 to 23, wherein R³ is

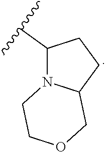 [SMILES: *C1CCC2COCCN12]

Embodiment 38: The compound of any one of embodiments 1 to 23, wherein R³ is

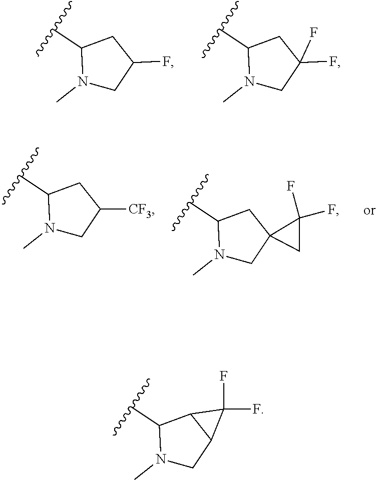

Embodiment 39: The compound of any one of embodiments 1 to 38, wherein R² is methyl.

Embodiment 40: The compound of any one of embodiments 1 to 38, wherein R² is CHF₂, CH₂F, or CF₃.

Embodiment 41: The compound of any one of embodiments 1 to 40, Ring A is R⁸-substituted or unsubstituted 3-10 membered heterocycle, or R⁸-substituted or unsubstituted 5-10 membered heteroaryl.

Embodiment 42: The compound of any one of embodiments 1 to 41, Ring A is R⁸-substituted or unsubstituted 3-6 membered heterocycle, or R⁸-substituted or unsubstituted 5-6 membered heteroaryl.

Embodiment 43: The compound of any one of embodiments 1 to 42, Ring A is R⁸-substituted or unsubstituted 3-6 membered heterocycle comprising at least 1 heterocyclic ring nitrogen atom, or R⁸-substituted or unsubstituted 5-6 membered heteroaryl comprising at least 1 heterocyclic ring nitrogen atom.

Embodiment 44: The compound of any one of embodiments 1 to 43, Ring A is R⁸-substituted or unsubstituted 5 or 6 membered heterocycle comprising at least 1 heterocyclic ring nitrogen atom.

Embodiment 45: The compound of any one of embodiments 1 to 44, Ring A is R⁸-substituted or unsubstituted 6 membered heteroaryl comprising at least 1 heterocyclic ring nitrogen atom.

Embodiment 46: The compound of any one of embodiments 1 to 45, Ring A is R⁸-substituted or unsubstituted pyridinyl, R⁸-substituted or unsubstituted pyrimidinyl or R⁸-substituted or unsubstituted pyrazinyl.

Embodiment 47: The compound of any one of claims 1 to 46, Ring A is a moiety of formula (H) or (H1) as described herein, wherein X is CH or N and n is 1 or 2.

Embodiment 48: The compound of any one of claims 1 to 47, Ring A is a moiety of formula;

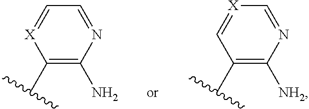

wherein X is CH or N.

Embodiment 49: The compound of any one of embodiments 1 to 48, wherein R⁵ and R⁶ are each hydrogen.

Embodiment 50: The compound of any one of embodiments 1 to 49, R⁵ is CH₃ and R⁶ is hydrogen.

Embodiment 51. A compound of Table 1 or a stereoisomer, atropisomer, tautomer, or pharmaceutically acceptable salt thereof.

Embodiment 52. A Compound of Table 2 or a stereoisomer, atropisomer, tautomer, or pharmaceutically acceptable salt thereof.

Embodiment 53. A Compound of Table 3 or a stereoisomer, atropisomer, tautomer, or pharmaceutically acceptable salt thereof.

Embodiment 54. A pharmaceutical composition comprising a compound or a stereoisomer, atropisomer, tautomer, or pharmaceutically acceptable salt thereof of any one of embodiments 1 to 53 and one or more pharmaceutically acceptable excipients.

Embodiment 55. A method of treating cancer, the method comprising administering an effective amount of a compound or a stereoisomer, atropisomer, tautomer, or pharmaceutically acceptable salt thereof of any one of embodiments 1 to 53 or a pharmaceutical composition of claim 54.

Embodiment 56. A method of treating cancer, the method comprising administering an effective amount of a compound or a stereoisomer, atropisomer, tautomer, or pharmaceutically acceptable salt thereof of any one of embodiments 1 to 53.

Embodiment 57. The method of embodiment 56, wherein the cancer is characterized as comprising a KRas mutation.

Embodiment 58. The method of embodiment 57, wherein the KRas mutation corresponds to a KRas^{G12D} mutation or KRas^{G12V} mutation.

Embodiment 59. The method of embodiment 57, further comprising testing a sample from the patient before administration for the absence or presence of a KRas mutation.

Embodiment 60. The method of embodiment 59, wherein the compound, stereoisomer, atropisomer, tautomer, or pharmaceutically acceptable salt thereof or pharmaceutical composition is administered to the patient after the patient sample shows the presence of a KRas mutation.

Embodiment 61. The method of any one of any one of embodiments 56 to 60, wherein the cancer is tissue agnostic.

Embodiment 62. The method of any one of any one of embodiments 56 to 60, wherein the cancer is pancreatic cancer, lung cancer, or colorectal cancer.

Embodiment 63. The method of embodiment 62, wherein the lung cancer is lung adenocarcinoma, NSCLC, or SCLC.

Embodiment 64. The method of embodiment 62, wherein the cancer is pancreatic cancer.

Embodiment 65. The method of embodiment 62, wherein the cancer is colorectal cancer.

Embodiment 66. The method of any one of any one of embodiments 56 to 65, further comprising administering at least one additional therapeutic agent.

Embodiment 67. The method of embodiment 66, wherein the additional therapeutic agent comprises an epidermal growth factor receptor (EGFR) inhibitor, phosphatidylinositol kinase (PI3K) inhibitor, insulin-like growth factor receptor (IGF1R) inhibitor, a Janus kinase (JAK) inhibitor, a Met kinase inhibitor, a SRC family kinase inhibitor, a mitogen-activated protein kinase (MEK) inhibitor, an extracellular-signal-regulated kinase (ERK) inhibitor, a topoisomerase inhibitor, a taxane, an anti-metabolite agent, or an alkylating agent.

Embodiment 68. A compound according to any one of embodiments 1 to 68, or a stereoisomer, atropisomer, tautomer, or pharmaceutically acceptable salt thereof, for use as therapeutically active substance.

Embodiment 69. The use of a compound according to any one of embodiments 1 to 53, or stereoisomer, atropisomer, tautomer, or pharmaceutically acceptable salt thereof, for the therapeutic treatment of a cancer comprising a KRas mutation.

Embodiment 70. The use of a compound according to any one of embodiments 1 to 53, or stereoisomer, atropisomer, tautomer, or pharmaceutically acceptable salt thereof, for the preparation of a medicament for the therapeutic treatment of a cancer comprising a KRas mutation.

Embodiment 71. Use of a compound of any one of embodiments 1 to 53, or stereoisomer, atropisomer, tautomer, or pharmaceutically salt thereof, in the manufacture of a medicament for inhibiting tumor metastasis.

Embodiment 72. A compound according to any one of embodiments 1 to 53, or stereoisomer, atropisomer, tautomer, or pharmaceutically salt thereof, for the therapeutic and/or prophylactic treatment of a cancer comprising a KRas mutation.

Embodiment 73. A method for regulating activity of a KRas mutant protein, the method comprising reacting the mutant protein with a compound of any one of embodiments 1 to 53, or stereoisomer, atropisomer, tautomer, or pharmaceutically acceptable salt thereof.

Embodiment 74. A method for inhibiting proliferation of a cell population, the method comprising contacting the cell population with the compound of any one of embodiments 1 to 53, or stereoisomer, atropisomer, tautomer, or pharmaceutically acceptable salt thereof.

Embodiment 75. The method of embodiment 74, wherein the inhibition of proliferation is measured as a decrease in cell viability of the cell population.

Embodiment 76. A method for preparing a labeled KRas mutant protein, the method comprising reacting a KRas mutant protein with a labeled compound of any one of embodiments 1 to 53, or stereoisomer, atropisomer, tautomer, or pharmaceutically acceptable salt thereof, to result in the labeled KRas mutant protein.

Embodiment 77. A method for inhibiting tumor metastasis comprising administering to an individual in need thereof a therapeutically effective amount of the compound of any one of embodiments 1 to 53, or stereoisomer, atropisomer, tautomer, or pharmaceutically acceptable salt thereof or a pharmaceutical composition of claim 55 to a subject in need thereof.

Embodiment 78. A process for synthesizing a compound of formula or (I) as set forth herein.

### Examples

The following examples illustrate the preparation and biological evaluation of compounds within the scope of the invention. These examples and preparations which follow are provided to enable those skilled in the art to more clearly understand and to practice the present invention. They should not be considered as limiting the scope of the invention, but merely as being illustrative and representative thereof.

### Example 1: 4-(10-((R)-1-(2-aminopyridin-3-yl)ethyl)-2-((3,3-difluoro-1-azabicyclo[3.2.0]heptan-5-yl)methoxy)-4-fluoro-9,10-dihydro-8H-7-oxa-1,3,6,10-tetraazacyclohepta[de]naphthalen-5-yl)-5-ethynyinaphthalen-2-ol

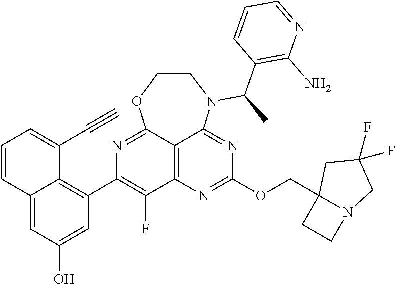 [SMILES: C#Cc1cccc2cc(O)cc(-c3nc4c5c(nc(OCC67CCN6CC(F)(F)C7)nc5c3F)N([C@H](C)c3cccnc3N)CCO4)c12]

### Synthetic Route

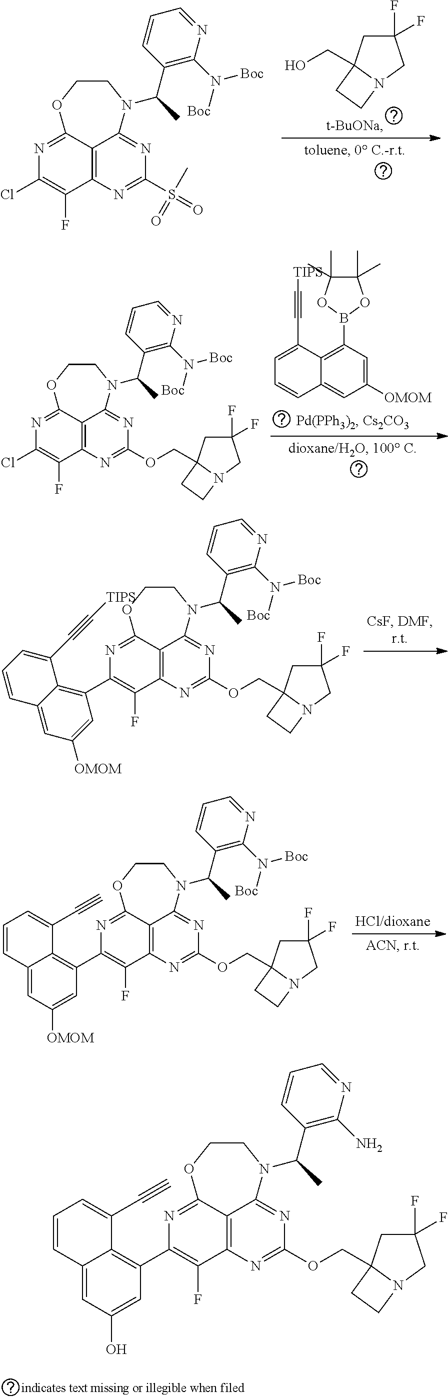

### Step 1: tert-butyl N-tert-butoxycarbonyl-N-[3-[(1R)-1-[7-chloro-3-[(3,3-difluoro-1-azabicyclo[3.2.0]heptan-5-yl)methoxy]-6-fluoro-10-oxa-2,4,8,13-tetrazatricyclo[7.4.1.05, 14]tetradeca-1,3,5(14),6,8-pentaen-13-yl]ethyl]-2-pyridyl]carbamate

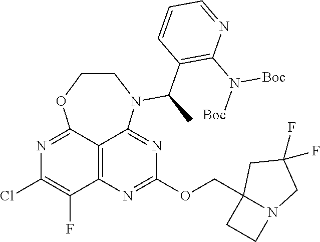 [SMILES: C[C@H](c1cccnc1N(C(=O)OC(C)(C)C)C(=O)OC(C)(C)C)N1CCOc2nc(Cl)c(F)c3nc(OCC45CCN4CC(F)(F)C5)nc1c23]

To a solution of (3,3-difluoro-1-azabicyclo[3.2.0]heptan-5-yl)methanol (315.3 mg, 1.93 mmol) in toluene (10 mL) was added sodium tert-butoxide (428.6 mg, 4.46 mmol) at 0° C. and stirred at 0° C. for 30 min. Then tert-butyl N-tert-butoxycarbonyl-N-[3-[(1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl]-2-pyridyl]carbamate (950.0 mg, 1.49 mmol) was added at 25° C. and stirred at 25° C. for 1.5 hours. After completion, the reaction was quenched with ammonium chloride saturated solution, concentrated under vacuum, diluted with dichloromethane, washed with water, dried over anhydrous sodium sulfate and concentrated under vacuum. The residue was purified by flash chromatography on silica gel eluting with dichloromethane/methanol (93:7) to afford tert-butyl N-tert-butoxycarbonyl-N-[3-[(1R)-1-[7-chloro-3-[(3,3-difluoro-1-azabicyclo[3.2.0]heptan-5-yl)methoxy]-6-fluoro-10-oxa-2,4,8,13-tetrazatricyclo[7.4.1.05, 14]tetradeca-1,3,5(14),6,8-pentaen-13-yl]ethyl]-2-pyridyl]carbamate (680.3 mg, 0.73 mmol, 49.4% yield) as a white solid. LC-MS: (ESI, m/Z): 722.3 [M+H]⁺

### Step 2: tert-butyl N-tert-butoxycarbonyl-N-[3-[(1R)-1-[3-[(3,3-difluoro-1-azabicyclo[3.2.0]heptan-5-yl)methoxy]-6-fluoro-7-[3-(methoxymethoxy)-8-(2-triisopropylsilylethynyl)-1-naphthyl]-10-oxa-2,4,8,13-tetrazatricyclo[7.4.1.05, 14]tetradeca-1,3,5(14),6,8-pentaen-13-yl]ethyl]-2-pyridyl]carbamate

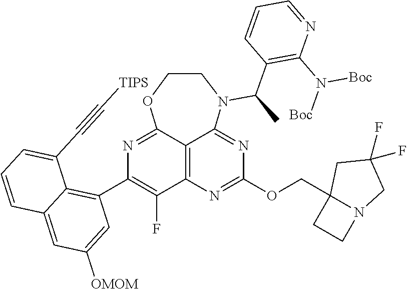

Under nitrogen, a solution of tert-butyl N-tert-butoxycarbonyl-N-[3-[(1R)-1-[7-chloro-3-[(3,3-difluoro-1-azabicyclo[3.2.0]heptan-5-yl)methoxy]-6-fluoro-10-oxa-2,4,8,13-tetrazatricyclo[7.4.1.05, 14]tetradeca-1,3,5(14),6,8-pentaen-13-yl]ethyl]-2-pyridyl]carbamate (630.0 mg, 0.68 mmol), tetrakis(triphenylphosphine)palladium(0) (157.3 mg, 0.14 mmol), cesium carbonate (669.3 mg, 2.04 mmol) and triisopropyl-[2-[6-(methoxymethoxy)-8-(4,4,5,5-tetramethyl-1,3,2-dioxaborolan-2-yl)-1-naphthyl]ethynyl]silane (403.8 mg, 0.82 mmol) in 1,4-dioxane (5 mL) and water (1 mL) stirred at 100° C. for 1 hour. After completion, the reaction mixture was diluted with dichloromethane, washed with water, dried over anhydrous sodium sulfate and concentrated under vacuum. The residue was purified by flash chromatography on silica gel eluting with dichloromethane/methanol (10:1) to afford tert-butyl N-tert-butoxycarbonyl-N-[3-[(1R)-1-[3-[(3,3-difluoro-1-azabicyclo[3.2.0]heptan-5-yl)methoxy]-6-fluoro-7-[3-(methoxymethoxy)-8-(2-triisopropylsilylethynyl)-1-naphthyl]-10-oxa-2,4,8,13-tetrazatricyclo[7.4.1.05, 14]tetradeca-1,3,5(14),6,8-pentaen-13-yl]ethyl]-2-pyridyl]carbamate (500.6 mg, 0.35 mmol, 50.9% yield) as a yellow solid. LC-MS: (ESI, m/Z): 1054.5 [M+H]⁺

### Step 3: tert-butyl N-tert-butoxycarbonyl-N-[3-[(1R)-1-[3-[(3,3-difluoro-1-azabicyclo[3.2.0]heptan-5-yl)methoxy]-7-[8-ethynyl-3-(methoxymethoxy)-1-naphthyl]-6-fluoro-10-oxa-2,4,8,13-tetrazatricyclo[7.4.1.05, 14]tetradeca-1,3,5(14),6,8-pentaen-13-yl]ethyl]-2-pyridyl]carbamate

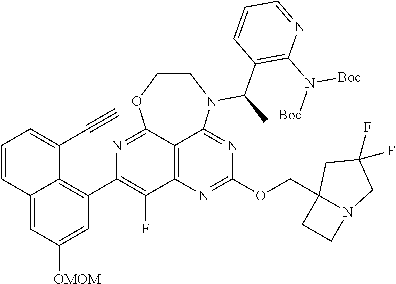

A solution of tert-butyl N-tert-butoxycarbonyl-N-[3-[(1R)-1-[3-[(3,3-difluoro-1-azabicyclo[3.2.0]heptan-5-yl)methoxy]-6-fluoro-7-[3-(methoxymethoxy)-8-(2-triisopropylsilylethynyl)-1-naphthyl]-10-oxa-2,4,8,13-tetrazatricyclo[7.4.1.05, 14]tetradeca-1,3,5(14),6,8-pentaen-13-yl]ethyl]-2-pyridyl]carbamate (480.0 mg, 0.33 mmol) and cesium fluoride (350.0 mg, 2.33 mmol) in N,N-dimethylformamide (3 mL) was stirred at 25° C. for 1 hour. After completion, the reaction mixture was diluted with water, extracted with ethyl acetate, washed with water dried over anhydrous sodium sulfate and concentrated under vacuum. The residue was purified by flash chromatography on silica gel eluting with dichloromethane/methanol (93:7) to afford tert-butyl N-tert-butoxycarbonyl-N-[3-[(1R)-1-[3-[(3,3-difluoro-1-azabicyclo[3.2.0]heptan-5-yl)methoxy]-7-[8-ethynyl-3-(methoxymethoxy)-1-naphthyl]-6-fluoro-10-oxa-2,4,8,13-tetrazatricyclo[7.4.1.05, 14]tetradeca-1,3,5(14),6,8-pentaen-13-yl]ethyl]-2-pyridyl]carbamate (310.5 mg, 0.25 mmol, 75.8% yield) as a yellow solid. LC-MS: (ESI, m/Z): 898.4 [M+H]⁺

### Step 4: 4-(10-((R)-1-(2-aminopyridin-3-yl)ethyl)-2-((3,3-difluoro-1-azabicyclo[3.2.0]heptan-5-yl)methoxy)-4-fluoro-9,10-dihydro-8H-7-oxa-1,3,6,10-tetraazacyclohepta[de]naphthalen-5-yl)-5-ethynylnaphthalen-2-ol

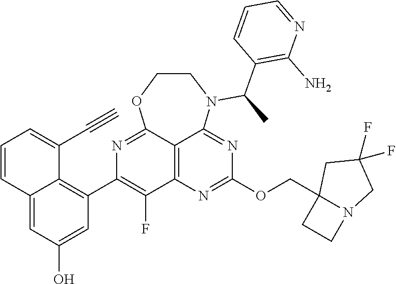 [SMILES: C#Cc1cccc2cc(O)cc(-c3nc4c5c(nc(OCC67CCN6CC(F)(F)C7)nc5c3F)N([C@H](C)c3cccnc3N)CCO4)c12]

A solution of tert-butyl N-tert-butoxycarbonyl-N-[3-[(1R)-1-[3-[(3,3-difluoro-1-azabicyclo[3.2.0]heptan-5-yl)methoxy]-7-[8-ethynyl-3-(methoxymethoxy)-1-naphthyl]-6-fluoro-10-oxa-2,4,8,13-tetrazatricyclo[7.4.1.05, 14]tetradeca-1,3,5(14),6,8-pentaen-13-yl]ethyl]-2-pyridyl]carbamate (300.0 mg, 0.33 mmol) in hydrogen chloride in 1,4-dioxane solution (3 mL, 12.00 mmol, 4.0 M in 1,4-dioxane) and acetonitrile (3 mL) was stirred for 5 hours at 25° C. After completion, the reaction mixture concentrated under vacuum, adjusted to pH>7 with saturated sodium bicarbonate solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: XBridge Shield RP18 OBD Column, 30*150 mm, 5 μm; Mobile Phase A: water (10 mmol/L NH₄HCO₃), Mobile Phase B: acetonitrile; Flow rate: 60 mL/min; Gradient: 43% B to 58% B in 10 min, 58% B; Wave Length: 254/220 nm; RT1(min): 7 to afford 4-(10-((R)-1-(2-aminopyridin-3-yl)ethyl)-2-((3,3-difluoro-1-azabicyclo[3.2.0]heptan-5-yl)methoxy)-4-fluoro-9,10-dihydro-8H-7-oxa-1, 3,6,10-tetraazacyclohepta[de]naphthalen-5-yl)-5-ethynylnaphthalen-2-ol (14.6 mg, 0.02 mmol, 6.6% yield) LC-MS: (ESI, m/Z): 654.3 [M+H]⁺

Example 1: ¹H NMR (300 MHz, DMSO-d₆, ppm) δ 10.14 (s, 1H), 7.98 (d, J=4.5 Hz, 1H), 7.88 (d, J=7.2 Hz, 1H), 7.65 (d, J=7.8 Hz, 1H), 7.53-7.34 (m, 2H), 7.32 (s, 1H), 7.10 (s, 1H), 6.79-6.60 (m, 1H), 6.49-6.23 (m, 1H), 5.93-5.65 (m, 2H), 4.55-4.17 (m, 4H), 3.88-3.71 (m, 1H), 3.70-3.61 (m, 1H), 3.62-3.43 (m, 1H), 3.28-3.06 (m, 3H), 2.79-2.54 (m, 2H), 2.47-2.23 (m, 3H), 1.61 (d, J=6.0 Hz, 3H). LC-MS: (ESI, m/Z): 654.3 [M+H]⁺.

### Example 2: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-ethynyl-6-fluoronaphthalen-2-ol Synthetic Route

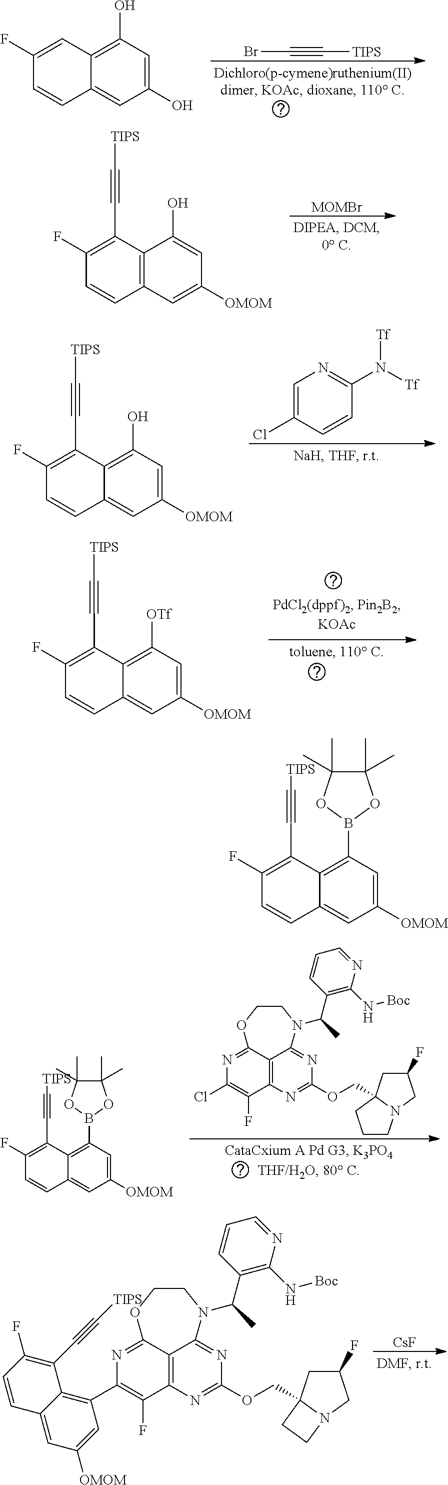

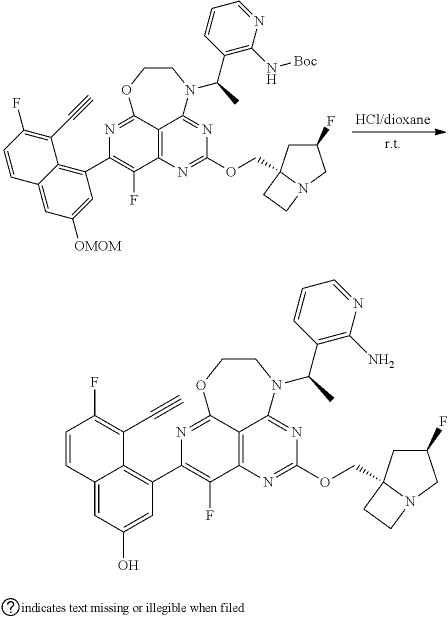

### Step 1: 7-8(triisopropylsilyl)ethynyl)napthalene-1,3-diol

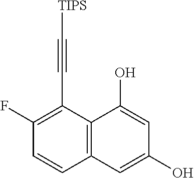

A solution of 7-fluoronaphthalene-1,3-diol (5.00 g, 28.1 mmol), (bromoethynyl)triisopropylsilane (11.00 g, 42.10 mmol), potassium acetate (5.51 g, 56.13 mmol) and dichloro(p-cymene)ruthenium(II)dimer (1.73 g, 2.81 mmol) in 1,4-dioxane (80 mL) was stirred at 110° C. for 2 hours. After completion, the solution was concentrated under vacuum,diluted with ethyl acetate, washed with water. The organic layer was dried over anhydrous sodium sulfate and concentrated under vacuum to afford 7-fluoro-8-((triisopropylsilyl)ethynyl)naphthalene-1,3-diol (8.7 g, crude) as a yellow solid. The crude product would be directly used in the next step without purification. LC-MS: (ESI, m/Z): 359.2 [M+H]⁺

### Step 2: 7-fluoro-3-(methoxymethoxy)-8-((triisopropylsilyl)ethynyl)naphthalen-1-ol

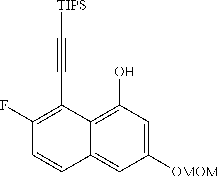

Under nitrogen, a solution of 7-fluoro-8-((triisopropylsilyl)ethynyl)naphthalene-1,3-diol (7.70 g, crude), bromo(methoxy)methane (4.03 g, 32.22 mmol) and N,N-diisopropylethylamine (8.33 g, 64.43 mmol) in dichloromethane (100 mL) stirred at 0° C. for 1.5 hours. After completion, the reaction solution was quenched with saturated ammonium chloride solution, diluted with ethyl acetate, washed with water, dried over anhydrous sodium sulfate and concentrated under vacuum. The residue was purified by flash chromatography on silica gel eluting with ethyl acetate/petroleum ether (3:97) to afford 7-fluoro-3-(methoxymethoxy)-8-((triisopropylsilyl)ethynyl)naphthalen-1-ol (6.60 g, 15.66 mmol, 72.9% yield) as yellow solid. LC-MS: (ESI, m/Z): 403.2 [M+H]⁺

### Step 3: 7-fluoro-3-(methoxymethoxy)-8-((triisopropylsilyl)ethynyl)naphthalen-1-yl trifluoromethanesulfonate

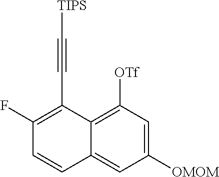

A solution of 7-fluoro-3-(methoxymethoxy)-8-((triisopropylsilyl)ethynyl)naphthalen-1-ol (5.00 g, 12.42 mmol) in tetrahydrofuran (50 mL) was added sodium hydride (546.6 mg, 13.66 mmol, 60% dispersion in mineral oil) at 0° C. and stirred at 25° C. for 15 min. Then N-(5-chloropyridin-2-yl)-1,1,1-trifluoro-N-((trifluoromethyl)sulfonyl)methanesulfonamide (5.85 g, 14.91 mmol) was added and stirred at 25° C. for 1 hour. After completion, the reaction solution was quenched with saturated ammonium chloride solution, diluted with ethyl acetate, washed with water, dried over anhydrous sodium sulfate and concentrated under vacuum. The residue was purified by flash chromatography on silica gel eluting with petroleum ether/ethyl acetate (10:1) to afford 7-fluoro-3-(methoxymethoxy)-8-((triisopropylsilyl)ethynyl)naphthalen-1-yl trifluoromethanesulfonate (5.10 g, 9.54 mmol, 76.8% yield) as a yellow oil. LC-MS: (ESI, m/Z): 535.1 [M+H]⁺

### Step 4: ((2-fluoro-6-(methoxymethoxy)-8-(4,4,5,5-tetramethyl-1,3,2-dioxaborolan-2-yl)naphthalen-1-yl)ethynyl)triisopropylsilane

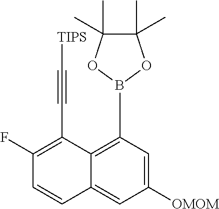

A solution of 7-fluoro-3-(methoxymethoxy)-8-((triisopropylsilyl)ethynyl)naphthalen-1-yl trifluoromethanesulfonate (700.0 mg, 1.31 mmol), 4,4,4′,4′,5,5,5′,5′-octamethyl-2,2′-bi(1,3,2-dioxaborolane) (499.0 mg, 1.96 mmol), [1,1′-bis(diphenylphosphino)ferrocene]dichloropalladium(II) (97.0 mg, 0.13 mmol) and potassium acetate (385.0 mg, 3.93 mmol) in toluene (15 mL) was stirred at 110° C. for 12 hours. After completion, the reaction solution was concentrated under vacuum, diluted with ethyl acetate, washed with water, dried over anhydrous sodium sulfate and concentrated under vacuum. The residue was purified by flash chromatography on silica gel eluting with petroleum ether/ethyl acetate (1:1) to afford ((2-fluoro-6-(methoxymethoxy)-8-(4,4,5,5-tetramethyl-1,3,2-dioxaborolan-2-yl)naphthalen-1-yl)ethynyl)triisopropylsilane (320.0 mg, 0.62 mmol, 47.6% yield) as yellow solid. LC-MS: (ESI, m/Z): 513.3 [M+H]⁺

### Step 5: tert-butyl (3-((R)-1-(4-fluoro-5-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate

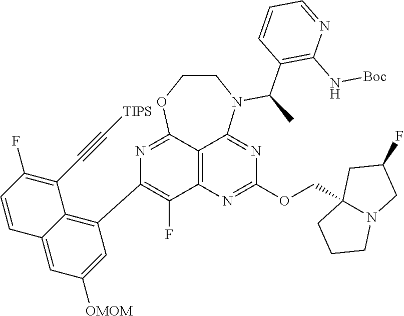

A solution of ((2-fluoro-6-(methoxymethoxy)-8-(4,4,5,5-tetramethyl-1,3,2-dioxaborolan-2-yl)naphthalen-1-yl)ethynyl)triisopropylsilane (256.9 mg, 0.50 mmol), tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (300.0 mg, 0.49 mmol), methanesulfonato(diadamantyl-n-butylphosphino)-2′-amino-1,1′-biphenyl-2-yl)palladium(II) dichloromethane adduct (30.4 mg, 0.04 mmol) and potassium phosphate tribasic (266.0 mg, 1.25 mmol) in tetrahydrofuran (5 mL) and water (1 mL) was stirred at 80° C. for 1.5 hours. After completion, the reaction mixture was diluted with dichloromethane, washed with water, dried over anhydrous sodium sulfate and concentrated under vacuum. The residue was purified by flash chromatography on silica gel eluting with petroleum ether/ethyl acetate (1:9) to afford tert-butyl (3-((R)-1-(4-fluoro-5-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (190.0 mg, 0.20 mmol, 42.6% yield) as a yellow oil. LC-MS: (ESI, m/Z): 1068.5 [M+H]⁺

### Step 6: tert-butyl (3-((R)-1-(5-(8-ethynyl-7-fluoro-3-(methoxymethoxy)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate

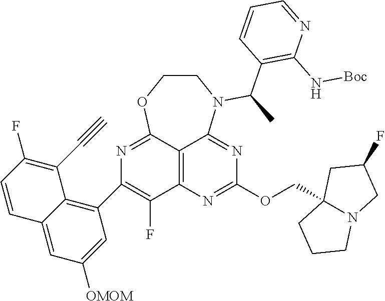

To a solution of tert-butyl (3-((R)-1-(4-fluoro-5-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (190.0 mg, 0.20 mmol) in N,N-dimethylformamide (4 mL) was added cesium fluoride (189.1 mg, 1.24 mmol) at 25° C. and stirred at 25° C. for 2 hours. After completion, the reaction was diluted with water, extracted with ethyl acetate, washed with water. The organic layer was dried over anhydrous sodium sulfate and concentrated under vacuum. The residue was purified by flash chromatography on silica gel eluting with dichloromethane/methanol (3:2) to afford the title compound (144.9 mg, 0.26 mmol, 89.3% yield) as a yellow solid. LC-MS: (ESI, m/Z): 912.4 [M+H]⁺

### Step 7: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-ethynyl-6-fluoronaphthalen-2-ol

A solution of tert-butyl (3-((R)-1-(5-(8-ethynyl-7-fluoro-3-(methoxymethoxy)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (190.0 mg, 0.21 mmol) in hydrochloric acid/1,4-dioxane (3 mL, 12.00 mmol, 4.0 M in 1,4-dioxane) was stirred for 0.5 hours at 25° C. After completion, the reaction mixture was concentrated under vacuum, diluted with ethyl acetate and adjusted to pH>7 with saturated sodium bicarbonate solution, extracted with ethyl acetate and the organic layer was combined. The organic layer was dried over anhydrous sodium sulfate and concentrated under vacuum. The product was further purified by Prep-HPLC with the following conditions: Column: Xselect CSH C18 OBD Column 30*150 mm 5 μm, n; Mobile Phase A: water (0.1% FA), Mobile Phase B: acetonitrile; Flow rate: 60 mL/min; Gradient: 9% B to 22% B in 8 min, 22% B; Wave Length: 254/220 nm; RT1(min): 8 to afford the title compound (26.4 mg, 0.04 mmol, 17.8% yield). LC-MS: (ESI, m/Z): 668.6 [M+H]⁺

Example 2: ¹H NMR (300 MHz, Methanol-d₄, ppm) δ 8.05-7.96 (m, 1H), 7.91-7.78 (m, 2H), 7.40-7.29 (m, 2H), 7.22 (d, J=2.5 Hz, 1H), 6.85 (ddd, J=7.4, 5.2, 2.1 Hz, 1H), 6.66-6.53 (m, 1H), 5.57 (d, J=51.9 Hz, 1H), 4.72 (s, 2H), 4.61-4.30 (m, 2H), 4.11-3.83 (m, 4H), 3.82-3.63 (m, 1H), 3.62-3.51 (m, 1H), 3.52-3.38 (m, 1H), 2.82-2.54 (m, 2H), 2.53-2.26 (m, 3H), 2.27-2.07 (m, 1H), 1.72 (dd, J=7.0, 2.3 Hz, 3H). LC-MS: (ESI, m/Z): 668.6 [M+H]⁺

### Example 3: 3-((R)-1-(5-(5-amino-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine Synthetic Route

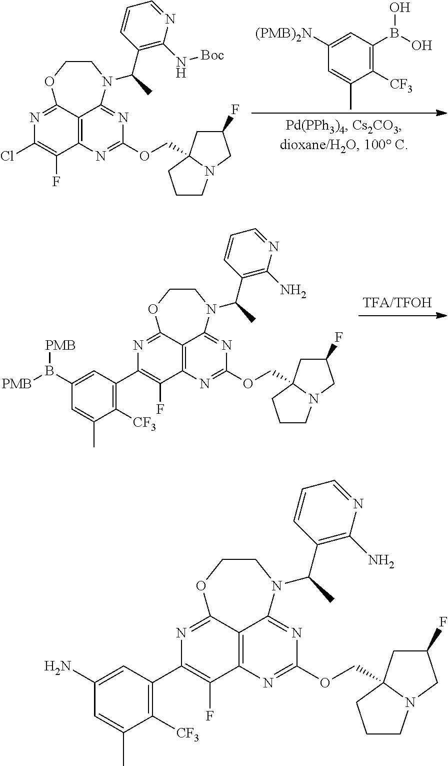

### Step 1: 3-((R)-1-(5-(5-(bis(4-methoxybenzyl)amino)-3-methyl-2-(trifluoromethyl) phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

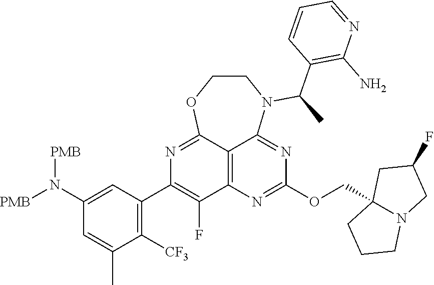

Under nitrogen, a solution of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (150.0 mg, 0.24 mmol), [5-[bis[(4-methoxyphenyl)methyl]amino]-3-methyl-2-(trifluoromethyl)phenyl]boronic acid (191.8 mg, 0.4 mmol), tetrakis(triphenylphosphine)palladium (24.1 mg, 0.02 mmol) and cesium carbonate (136.1 mg, 0.4 mmol) in dioxane/water (5/1) (2 mL) was stirred at 100° C. for 2 hours. After completion, the filtrate was concentrated under reduced pressure. The reaction mixture was diluted with water. The resulting solution was extracted with ethyl acetate and the organic layers were combined. The organic layer was dried over anhydrous sodium sulfate and concentrated under vacuum. The residue was purified by flash chromatography on silica gel eluting with dichloromethane/methanol (10:1) to afford the title compound (60.0 mg, 0.1 mmol, 32% yield) as a yellow solid. LC-MS: (ESI, m/Z): 897.4 [M+H]⁺

### Step 2: 3-((R)-1-(5-(5-amino-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

A solution of 3-((R)-1-(5-(5-(bis(4-methoxybenzyl)amino)-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (60.0 mg, 0.07 mmol) in trifluoroacetic acid/trifluoromethanesulfonic acid (10/1) (0.5 mL) was stirred at 25° C. for 1 hour. After completion, the reaction mixture was adjusted to pH=7 with saturated sodium bicarbonate solution. The resulting solution was extracted with dichloromethane and the organic layers were combined. The organic layer was dried over anhydrous sodium sulfate and concentrated under vacuum. The residue was purified by flash chromatography on silica gel eluting with methanol/dichloromethane (1:30) to afford crude solid. The crude product was purified by Prep-HPLC with the following conditions: (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 29% B to 54% B in 9 min, 54% B; Wave Length: 254/220) to afford the title compound (8.6 mg, 0.01 mmol, 19.6% yield). LC-MS: (ESI, m/Z): 657.3 [M+H]⁺

Example 3: ¹H NMR (300 MHz, DMSO-d₆, ppm) δ 7.96 (dd, J=4.9, 1.7 Hz, 1H), 7.62 (dd, J=7.6, 1.8 Hz, 1H), 6.66 (dd, J=7.5, 4.9 Hz, 1H), 6.59 (d, J=2.3 Hz, 1H), 6.41-6.24 (m, 2H), 5.86 (s, 2H), 5.70 (s, 2H), 5.28 (d, J=54.3 Hz, 1H), 4.41 (dd, J=12.7, 6.3 Hz, 1H), 4.28 (dd, J=12.7, 6.4 Hz, 1H), 4.12 (s, 2H), 3.71 (dd, J=15.9, 6.4 Hz, 1H), 3.52-3.36 (m, 1H), 3.20-3.02 (m, 2H), 3.00 (s, 1H), 2.91-2.76 (m, 1H), 2.35 (d, J=2.3 Hz, 3H), 2.25-2.11 (m, 1H), 2.10-1.93 (m, 2H), 1.92-1.70 (m, 3H), 1.55 (d, J=6.8 Hz, 3H).

### Example 4: 3-((R)-1-(5-(3-amino-8-ethynyinaphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Synthetic Route

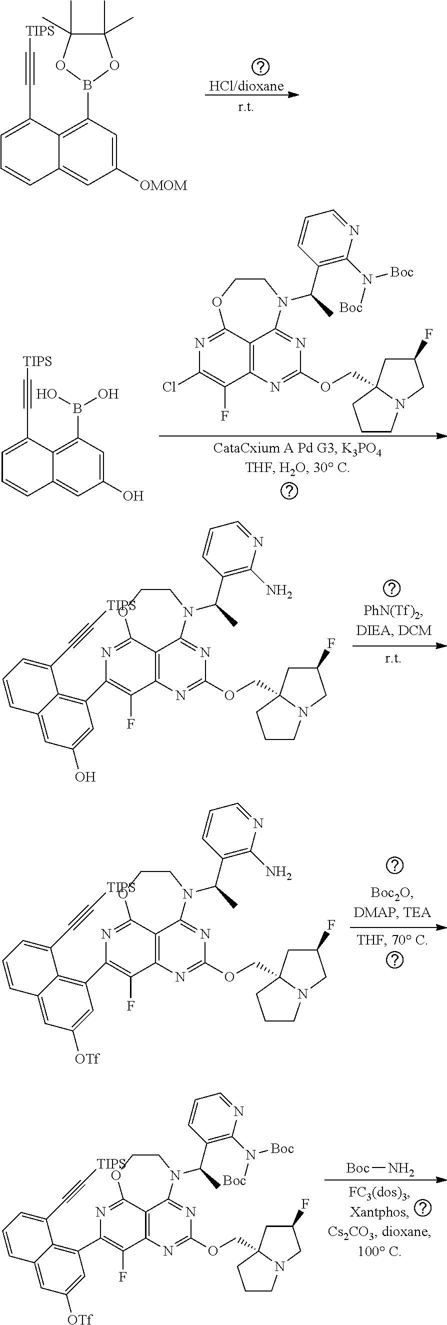

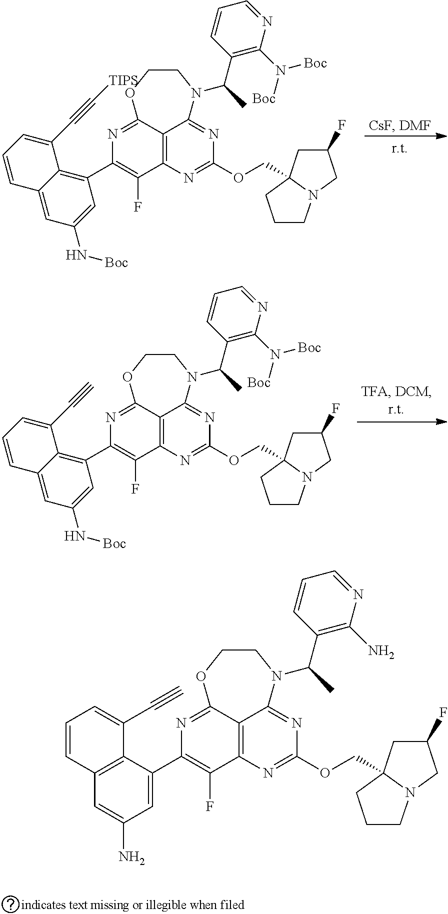

### Step 1: (3-hydroxy-8-((triisopropylsilyl)ethynyl)naphthalen-1-yl)boronic acid

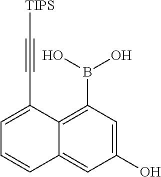

A solution of triisopropyl ((6-(methoxymethoxy)-8-(4,4,5,5-tetramethyl-1,3,2-dioxaborolan-2-yl)naphthalen-1-yl)ethynyl)silane (700.0 mg, 1.42 mmol) in hydrochloric acid/1,4-dioxane (4.86 mL, 19.44 mmol, 4.0 M in 1,4-dioxane) was stirred at 25° C. for 1 hour. After completion, the reaction mixture was concentrated under vacuum. The residue was purified by flash chromatography on silica gel eluting with petroleum ether/ethyl acetate (9:1) to afford the title compound (575.3 mg, 1.40 mmol, 99.3% yield) as a white solid. LC-MS: (ESI, m/Z): 369.2 [M+H]⁺

### Step 2: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-((triisopropylsilyl)ethynyl)naphthalen-2-ol

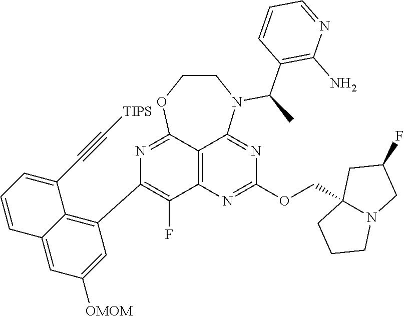

Under nitrogen, a solution of (3-hydroxy-8-((triisopropylsilyl)ethynyl)naphthalen-1-yl)boronic acid (420.1 mg, 1.14 mmol), tert-butyl N-tert-butoxycarbonyl-N-[3-[(1R)-1-[7-chloro-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]carbamate (630.0 mg, 0.88 mmol), [(di(1-adamantyl)-butylphosphine)-2-(2′-amino-1,1′-biphenyl)]palladium(II) methanesulfonate (63.9 mg, 0.09 mmol) and potassium phosphate tribasic (558.6 mg, 2.63 mmol) in tetrahydrofuran (5 mL) and water (1 mL) was stirred at 80° C. for 1 hour. After completion, the reaction was diluted with ethyl acetate, washed with water, dried over anhydrous sodium sulfate and concentrated under vacuum. The residue was purified by flash chromatography on silica gel eluting with petroleum ether/ethyl acetate (7:3) to afford the title compound (300.0 mg, 0.24 mmol, 27.9% yield) as a white solid. LC-MS: (ESI, m/Z): 806.4 [M+H]⁺

### Step 3: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-((triisopropylsilyl)ethynyl)naphthalen-2-yl trifluoromethanesulfonate

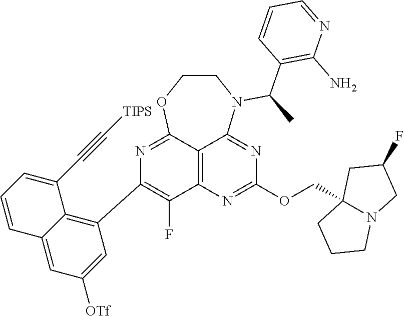

A solution of 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-((triisopropylsilyl)ethynyl)naphthalen-2-ol (255.0 mg, 0.33 mmol), N-phenylbis(trifluoromethanesulfonimide) (147.3 mg, 0.42 mmol) and N,N-diisopropylethylamine (122.7 mg, 0.96 mmol) in dichloromethane (5 mL) was stirred at 25° C. for 2.5 hours. After completion, the reaction was concentrated under vacuum. The residue was purified by flash chromatography on reverse-phase column eluting with water/acetonitrile (1:49) to afford the title compound (165.0 mg, 0.18 mmol, 52.8% yield) as an orange oil. LC-MS: (ESI, m/Z): 938.3 [M+H]⁺

### Step 4: [4-[13-[(1R)-1-[2-[bis(tert-butoxycarbonyl)amino]-3-pyridyl]ethyl]-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-7-yl]-5-(2-triisopropylsilylethynyl)-2-naphthyl]trifluoromethanesulfonate

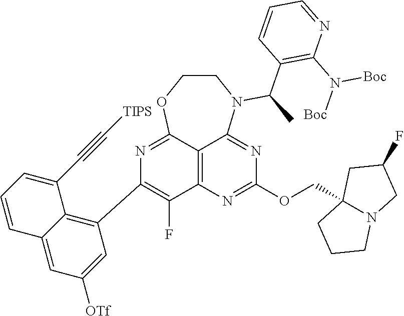

A solution of triethylamine (42.1 mg, 0.42 mmol), 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-((triisopropylsilyl)ethynyl)naphthalen-2-yl trifluoromethanesulfonate (130.0 mg, 0.14 mmol), 4-dimethylaminopyridine (16.9 mg, 0.14 mmol) and di-tert-butyldicarbonate (151.2 mg, 0.69 mmol) in tetrahydrofuran (3 mL) was stirred for 1 hour at 70° C. After completion, the reaction was diluted with dichloromethane, washed with water, dried over anhydrous sodium sulfate and concentrated under vacuum. The residue was purified by flash chromatography on silica gel eluting with dichloromethane/ethyl acetate (8:1) to afford the title compound (140.0 mg, 0.10 mmol, 72.8% yield) as a white solid. LC-MS: (ESI, m/Z): 1138.4 [M+H]⁺

### Step 5: tert-butyl (tert-butoxycarbonyl)(3-((R)-1-(5-(3-((tert-butoxycarbonyl)amino)-8-((triisopropylsilyl)ethynyl)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate

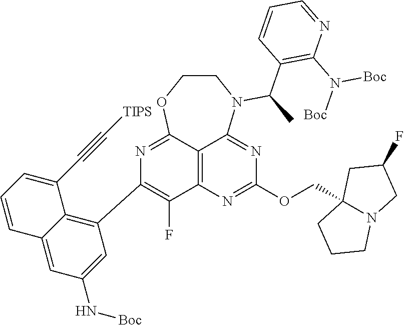

Under nitrogen, a solution of tris(dibenzylideneacetone)dipalladium (9.2 mg, 0.01 mmol), cesium carbonate (98.6 mg, 0.30 mmol), 4,5-bis(diphenylphosphino)-9,9-dimethylxanthene (11.7 mg, 0.02 mmol), [4-[13-[(1R)-1-[2-[bis(tert-butoxycarbonyl)amino]-3-pyridyl]ethyl]-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-7-yl]-5-(2-triisopropylsilylethynyl)-2-naphthyl]trifluoromethanesulfonate (140.0 mg, 0.10 mmol) and tert-butyl carbamate (11.8 mg, 0.10 mmol) in 1,4-dioxane (2 mL) was stirred at 100° C. for 2 hours. After completion, the reaction mixture was diluted with ethyl acetate, washed with water, dried over anhydrous sodium sulfate and concentrated under vacuum. The residue was purified by flash chromatography on silica gel eluting with petroleum ether/ethyl acetate (1:1) to afford the title compound (70.2 mg, 0.06 mmol, 55.9% yield) as a yellow oil. LC-MS: (ESI, m/Z): 1105.6 [M+H]⁺

### Step 6: tert-butyl (tert-butoxycarbonyl)(3-((R)-1-(5-(3-((tert-butoxycarbonyl)amino)-8-ethynylnaphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate

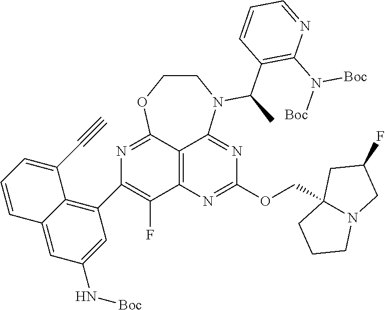 [SMILES: C#Cc1cccc2cc(NC(=O)OC(C)(C)C)cc(-c3nc4c5c(nc(OC[C@@]67CCCN6C[C@H](F)C7)nc5c3F)N([C@H](C)c3cccnc3N(C(=O)OC(C)(C)C)C(=O)OC(C)(C)C)CCO4)c12]

A solution of tert-butyl (tert-butoxycarbonyl)(3-((R)-1-(5-(3-((tert-butoxycarbonyl)amino)-8-((triisopropylsilyl)ethynyl)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (70.0 mg, 0.06 mmol) and cesium fluoride (0.06 g, 0.39 mmol) in N,N-dimethylformamide (2 mL) was stirred at 25° C. for 1 hour. After completion, the reaction mixture was diluted with water, extracted with ethyl acetate, washed with water, dried over anhydrous sodium sulfate and concentrated under vacuum. The residue was purified by flash chromatography on silica gel eluting with dichloromethane/methanol (49:1) to afford the title compound (55.2 mg, 0.05 mmol, 94.6% yield). LC-MS: (ESI, m/Z): 949.4 [M+H]⁺

### Step 7: 3-((R)-1-(5-(3-amino-8-ethynylnaphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

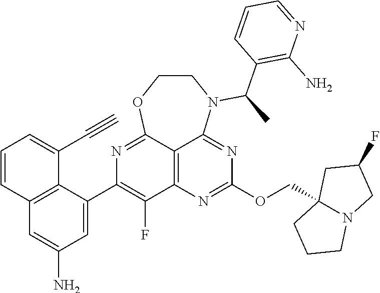 [SMILES: C#Cc1cccc2cc(N)cc(-c3nc4c5c(nc(OC[C@@]67CCCN6C[C@H](F)C7)nc5c3F)N([C@H](C)c3cccnc3N)CCO4)c12]

A solution of tert-butyl (tert-butoxycarbonyl)(3-((R)-1-(5-(3-((tert-butoxycarbonyl)amino)-8-ethynylnaphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (50.0 mg, 0.05 mmol) in dichloromethane (2 mL) and trifluoroacetic acid (0.4 mL) was stirred at 25° C. for 1 hour. After completion, the reaction mixture was concentrated under vacuum, diluted with dichloromethane, adjusted to pH>7 with saturated sodium bicarbonate solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with following conditions: Column: XBridge Shield RP18 OBD Column, 30*150 mm, 5 μm; Mobile Phase A: water (10 mmol/L NH₄HCO₃), Mobile Phase B: acetonitrile; Flow rate: 60 mL/min; Gradient: 34% B to 48% B in 8 min, 48% B; Wave Length: 254/220 nm; RT1(min): 8 to afford the title compound (5.3 mg, 0.01 mmol, 14.9% yield). LC-MS: (ESI, m/Z): 649.3 [M+H]⁺

Example 4: ¹H NMR (300 MHz, Methanol-d₄, ppm) δ 7.96 (d, J=5.1Hz, 1H), 7.76 (d, J=7.4, 1.5 Hz, 1H), 7.71-7.62 (m, 1H), 7.38 (dd, J=7.1, 3.6, 1.4 Hz, 1H), 7.34-7.24 (m, 1H), 7.14 (d, J=2.4 Hz, 1H), 7.07 (t, J=2.6 Hz, 1H), 6.84-6.74 (m, 1H), 6.67-6.52 (m, 1H), 5.31 (d, J=53.5 Hz, 1H), 4.53-4.37 (m, 2H), 4.38-4.20 (m, 2H), 3.85-3.66 (m, 1H), 3.64-3.39 (m, 1H), 3.29-3.12 (m, 3H), 3.10-2.94 (m, 2H), 2.51-2.08 (m, 3H), 2.07-1.80 (m, 3H), 1.67 (d, J=6.9 Hz, 3H). LC-MS: (ESI, m/Z): 649.3 [M+H]⁺

### Example 5: 3-((R)-1-(5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Synthetic Route

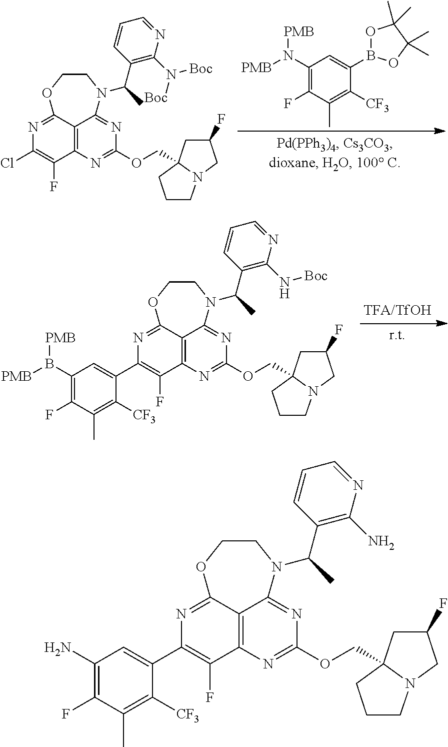

### Step 1: tert-butyl (3-((R)-1-(5-(5-(bis(4-methoxybenzyl)amino)-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate

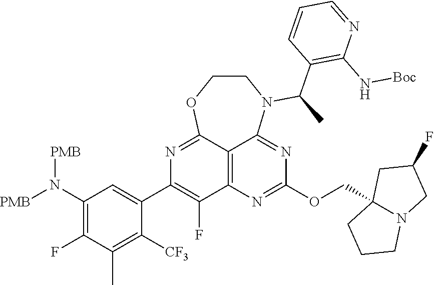

Under nitrogen, a solution of tert-butyl N-tert-butoxycarbonyl-N-(3-(rac-(1R)-1-(7-chloro-6-fluoro-3-((rac-(2R,8S)-2-fluoro-1,2,3,5,6,7-hexahydropyrrolizin-8-yl)methoxy)-10-oxa-2,4,8,13-tetrazatricyclo[7.4.1.05, 14]tetradeca-1,3,5(14),6,8-pentaen-13-yl)ethyl)-2-pyridyl)carbamate (130.0 mg, 0.18 mmol) and 2-fluoro-N,N-bis(4-methoxybenzyl)-3-methyl-5-(4,4,5,5-tetramethyl-1,3,2-dioxaborolan-2-yl)-4-(trifluoromethyl)aniline (121.0 mg, 0.22 mmol) in 1,4-dioxane (2.0 mL) and water (0.2 mL) was added cesium carbonate (118.0 mg, 0.36 mmol) and tetrakis(triphenylphosphine)palladium (21.0 mg, 0.02 mmol) and stirred for 3 hours at 100° C. After completion, the resulting solution was diluted with ethyl acetate and washed with water. The organic layer was washed with brine and dried over anhydrous sodium sulfate and concentrated under vacuum. The residue was purified by flash chromatography on silica gel eluting with dichloromethane/methanol (9:1) to afford the title compound (158.0 mg, 0.15 mmol, 78.3% yield) as a brown solid. LC-MS: (ESI, m/Z): 1115.5 [M+H]⁺

### Step 2: 3-((R)-1-(5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

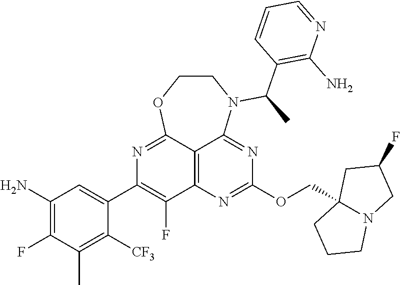 [SMILES: Cc1c(F)c(N)cc(-c2nc3c4c(nc(OC[C@@]56CCCN5C[C@H](F)C6)nc4c2F)N([C@H](C)c2cccnc2N)CCO3)c1C(F)(F)F]

A solution of tert-butyl (3-((R)-1-(5-(5-(bis(4-methoxybenzyl)amino)-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (158.0 mg, 0.15 mmol) in trifluoroacetic acid (1.5 mL) and trifluoromethanesulfonic acid (0.15 mL) was stirred at 25° C. for 15 minutes. After completion, the mixture was concentrated under vacuum. The residue was diluted with dichloromethane and adjusted to pH=7 by saturated sodium bicarbonate solution. The mixture was washed with water and brine. The organic layer was dried over anhydrous sodium sulfate and concentrated under vacuum. The crude product was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 34% B to 59% B in 9 min, 59% B; Wave Length: 254/220 nm; RT1(min): 8.6 to afford the title compound (26.0 mg, 0.04 mmol, 27.2% yield) as a white solid. LC-MS: (ESI, m/Z): 675.2 [M+H]⁺

Example 5: ¹H NMR (300 MHz, DMSO-d₆, ppm) δ 7.96 (dd, J=4.9, 1.7 Hz, 1H), 7.62 (dd, J=7.5, 1.8 Hz, 1H), 6.67 (dd, J=7.5, 4.9 Hz, 1H), 6.54 (d, J=8.7 Hz, 1H), 6.34 (q, J=6.7 Hz, 1H), 6.01 (s, 2H), 5.71 (s, 2H), 5.29 (d, J=54.5 Hz, 1H), 4.42 (dd, J=12.9, 6.3 Hz, 1H), 4.28 (dd, J=12.6, 6.3 Hz, 1H), 4.13 (s, 2H), 3.72 (dd, J=16.0, 6.4 Hz, 1H), 3.46-3.35 (m, 1H), 3.23-2.93 (m, 3H), 2.93-2.78 (m, 1H), 2.31 (s, 3H), 2.22-1.96 (m, 3H), 1.96-1.72 (m, 3H), 1.55 (d, J=6.8 Hz, 3H). LC-MS: (ESI, m/Z): 675.2 [M+H]⁺

### Example 6: 6-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-4-methyl-5-(trifluoromethyl)pyridin-2-amine

### Synthetic Route

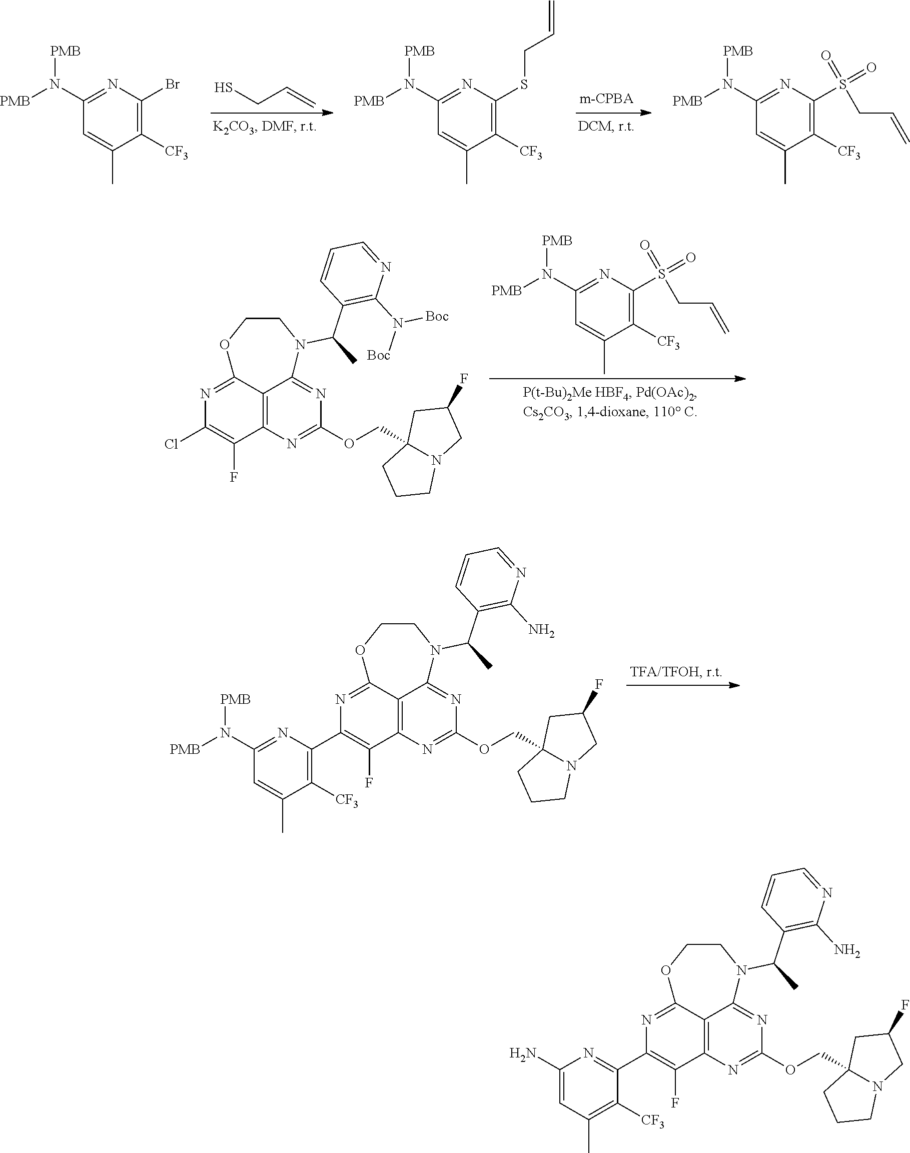

### Step 1: 6-(allylthio)-N,N-bis(4-methoxybenzyl)-4-methyl-5-(trifluoromethyl)pyridin-2-amine

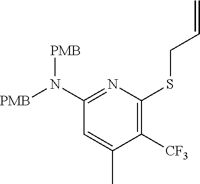

Under nitrogen, a mixture of 6-bromo-N,N-bis(4-methoxyphenyl)methyl-4-methyl-5-(trifluoromethyl)pyridin-2-amine (5.0 g, 10.09 mmol) and potassium carbonate (2.8 g, 20.21 mmol) in N,N-dimethylformamide (48 mL) was stirred at 25° C. for 5 minutes. Then allyl mercaptan (4.03 mL, 50.58 mmol) was added and stirred at 25° C. for 2 days. After completion, the resulting solution was diluted with ethyl acetate and washed with water. The organic layer was washed with brine and dried over anhydrous sodium sulfate and concentrated under vacuum. The residue was purified by flash chromatography on silica gel eluting with petroleum ether/ethyl acetate (10:1) to afford the title compound (4.89 g, 10.01 mmol, 99.2% yield) as a yellow solid. LC-MS: (ESI, m/Z): 489.2 [M+H]⁺

### Step 2:6-(allylsulfonyl)-N,N-bis(4-methoxybenzyl)-4-methyl-5-(trifluoromethyl)pyridin-2-amine

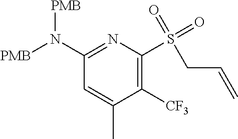

A solution of 6-(allylthio)-N,N-bis(4-methoxybenzyl)-4-methyl-5-(trifluoromethyl)pyridin-2-amine (4.92 g, 10.07 mmol) and 3-chloroperoxybenzoic acid (5.16 g, 29.90 mmol) in dichloromethane (40 mL) was stirred at 25° C. for 4 hours. Then 3-chloroperoxybenzoic acid (1.72 g, 9.97 mmol) was added and stirred at 25° C. for 1 hour. After completion, the solvent was added saturated sodium sulfite solution and stirred for 5 min. Then the solution was diluted with dichloromethane and washed with water. The organic layer was dried over anhydrous sodium sulfate and concentrated under vacuum. The residue was purified by flash chromatography on silica gel eluting with petroleum ether/ethyl acetate (1:1) to afford the title compound (1.12 g, 2.15 mmol, 21.4% yield) as a white solid. LC-MS: (ESI, m/Z): 520.9 [M+H]⁺

### Step 3:6-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-4-methyl-5-(trifluoromethyl)pyridin-2-amine

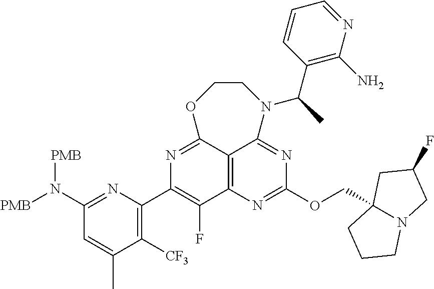

Under nitrogen, a mixture of tert-butyl N-tert-butoxycarbonyl-N-(3-(rac-(1R)-1-(7-chloro-6-fluoro-3-((rac-(2R, 8S)-2-fluoro-1,2,3,5,6,7-hexahydropyrrolizin-8-yl)methoxy)-10-oxa-2,4, 8,13-tetrazatricyclo(7.4.1.05, 14)tetradeca-1, 3, 5(14), 6, 8-pentaen-13-yl)ethyl)-2-pyridyl)carbamate (300.0 mg, 0.42 mmol), 6-(allylsulfonyl)-N,N-bis(4-methoxybenzyl)-4-methyl-5-(trifluoromethyl) pyridin-2-amine (330.0 mg, 0.64 mmol), palladium (II) acetate (10.0 mg, 0.04 mmol), cesium carbonate (270.0 mg, 0.82 mmol) and methyl (bis(2-methyl-2-propanyl))phosphonium tetrafluoroborate (22.0 mg, 0.09 mmol) in 1,4-dioxane (6 mL) was added and stirred at 110° C. for 12 hours. After completion, the resulting solution was diluted with ethyl acetate and washed with water. The organic layer was washed with brine and dried over anhydrous sodium sulfate and concentrated under vacuum. The residue was purified by flash chromatography on silica gel eluting with dichloromethane/methanol (93:7) to afford the title compound (163.0 mg, 0.18 mmol, 43.5% yield) as a yellow solid. LC-MS: (ESI, m/Z): 898.3 [M+H]⁺

### Step 4: 6-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-4-methyl-5-(trifluoromethyl) pyridin-2-amine

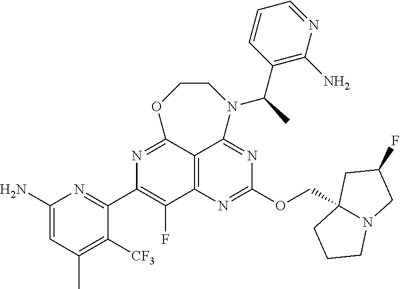 [SMILES: Cc1cc(N)nc(-c2nc3c4c(nc(OC[C@@]56CCCN5C[C@H](F)C6)nc4c2F)N([C@H](C)c2cccnc2N)CCO3)c1C(F)(F)F]

A solution of 6-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-4-methyl-5-(trifluoromethyl)pyridin-2-amine (163.0 mg, 0.18 mmol) in trifluoroacetic acid (5 mL) and trifluoromethanesulfonic acid (0.5 mL) was stirred at 25° C. for 0.5 hours. After completion, the mixture was concentrated under vacuum. The residue was diluted with dichloromethane and adjusted to pH=7 by saturated sodium bicarbonate solution. The mixture was washed with water. The organic layer was washed with brine and dried over anhydrous sodium sulfate and concentrated under vacuum. The crude product was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9; to afford the title compound (24.1 mg, 0.037 mmol, 20.2% yield). LC-MS: (ESI, m/Z): 658.2 [M+H]⁺

Example 6: ¹H NMR (300 MHz, DMSO-d₆, ppm) δ 7.96 (dd, J=4.9, 1.7 Hz, 1H), 7.62 (dd, J=7.6, 1.8 Hz, 1H), 6.77 (s, 2H), 6.66 (dd, J=7.5, 4.9 Hz, 1H), 6.47 (s, 1H), 6.33 (q, J=6.8 Hz, 1H), 5.70 (s, 2H), 5.29 (d, J=54.3 Hz, 1H), 4.41 (dd, J=12.8, 6.4 Hz, 1H), 4.28 (dd, J=12.8, 6.3 Hz, 1H), 4.13 (s, 2H), 3.71 (dd, J=15.9, 6.4 Hz, 1H), 3.47-3.36 (m, 1H), 3.22-2.93 (m, 3H), 2.92-2.76 (m, 1H), 2.35 (d, J=2.2 Hz, 3H), 2.28-2.11 (m, 1H), 2.12-1.93 (m, 2H), 1.93-1.68 (m, 3H), 1.56 (d, J=6.8 Hz, 3H). LC-MS: (ESI, m/Z): 658.2 [M+H]⁺

### Example 7: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-ethynyinaphthalen-2-ol

### Synthetic Route

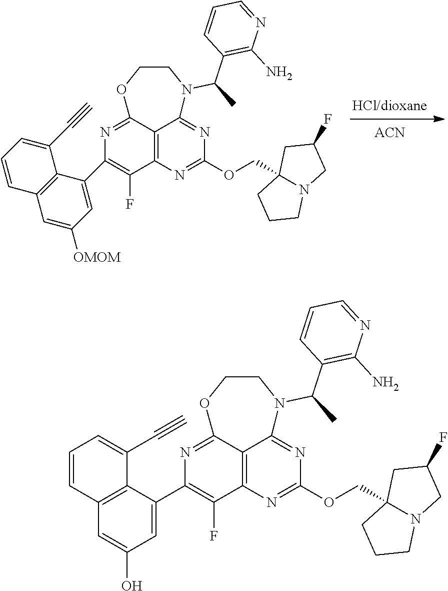

Step 1: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-ethynylnaphthalen-2-ol

A solution of 3-((R)-1-(5-(8-ethynyl-3-(methoxymethoxy)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (91.0 mg, 0.13 mmol) and hydrochloric acid (0.5 mL, 2.00 mmol, 4 mol/L solution in 1,4-dioxane) in acetonitrile (1 mL) was stirred at 25° C. for 0.5 hours. After completion, the reaction mixture was concentrated under vacuum. The crude product was purified by Prep-HPLC with the following conditions: Column: Xselect CSH C18 OBD Column 30*150 mm 5 μm; Mobile Phase A: Water(0.1% FA), Mobile Phase B: ACN; Flow rate: 60 mL/min; Gradient: 8% B to 24% B in 8 min, 24% B; Wave Length: 254/220 nm; RT1(min): 7.5 to afford the title compound (13.0 mg,0.02 mmol, 19.7% yield). LC-MS: (ESI, m/Z): 650.2 [M+H]⁺

Example 7: ¹H NMR (300 MHz, Methanol-d₄, ppm) δ 8.26 (s, 1H), 7.99-7.88 (m, 1H), 7.77 (d, J=2.3 Hz, 1H), 7.69 (d, J=8.0 Hz, 2H), 7.56-6.81 (m, 2H), 6.75 (t, J=6.4 Hz, 1H), 6.44 (d, J=7.1Hz, 1H), 5.42 (d, J=52.9 Hz, 1H), 4.52-4.05 (m, 4H), 3.80-3.38 (m, 5H), 3.22 (s, 1H), 2.59-1.89 (m, 7H), 1.57 (d, J=6.7 Hz, 3H).

### Example 8: 3-((R)-1-(5-(5-amino-4-fluoro-3-methyl-2-(trifluoromethyl)phenyl)-2-(((S)-4,4-difluoro-1-methylpyrrolidin-2-yl)methoxy)-4-fluoro-8,9-dihydro-10H-7-oxa-1,3,6,10-tetraazacyclohepta[de]naphthalen-10-yl)ethyl)pyrazin-2-amine Synthetic Route

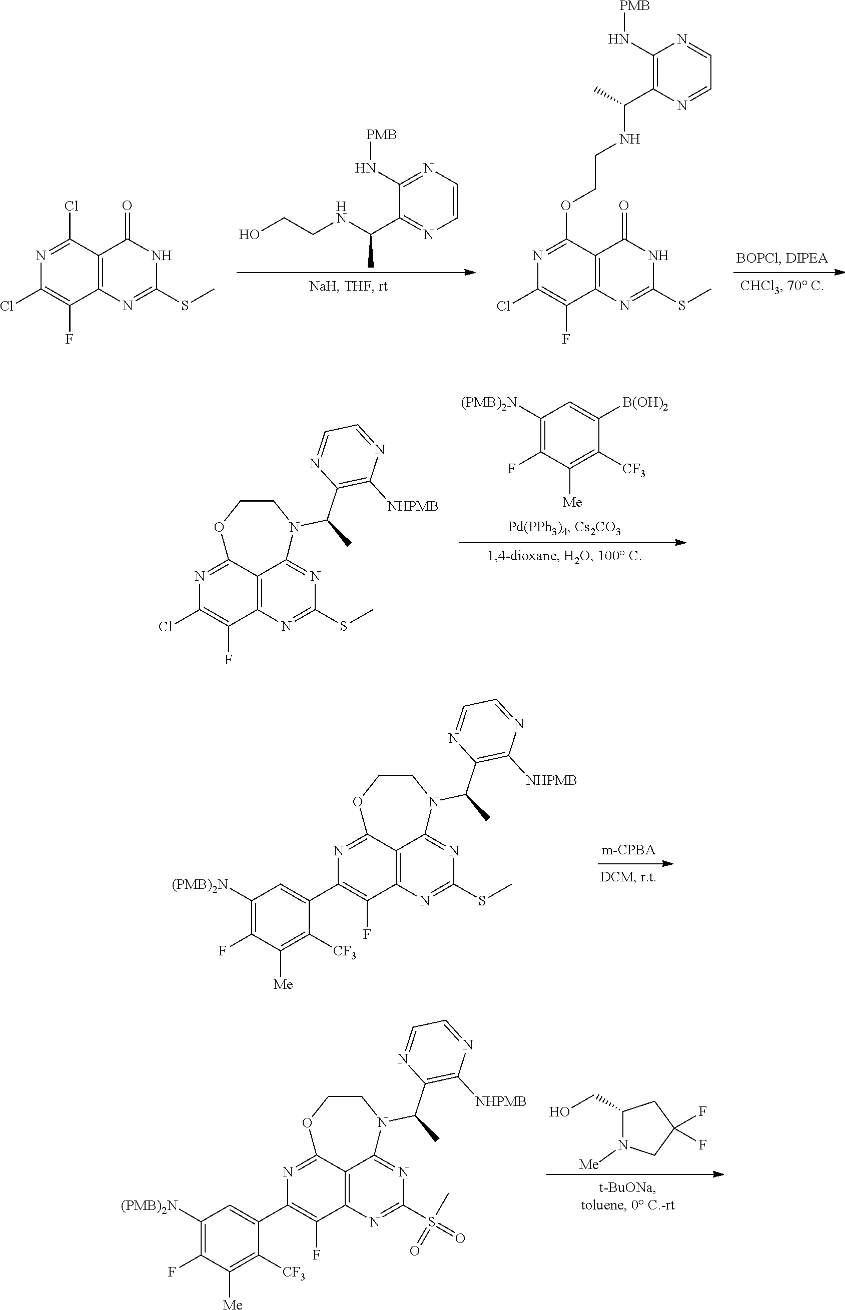

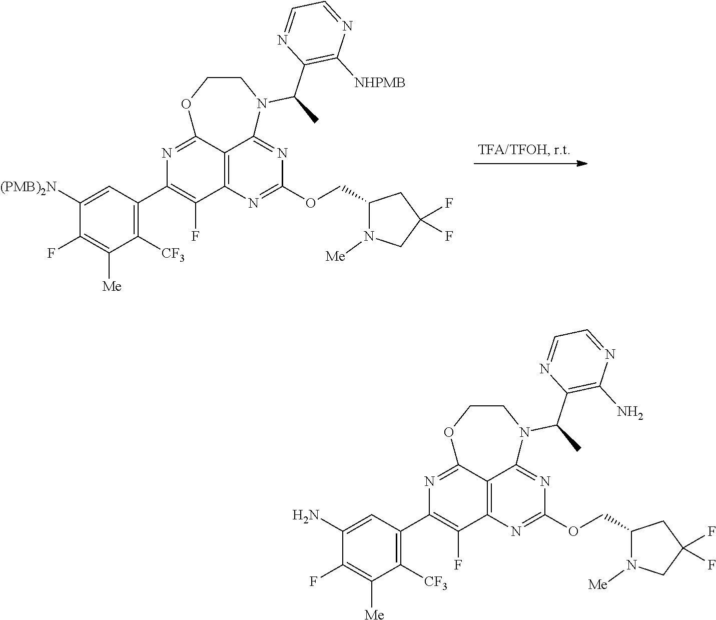

### Step 1: (R)-7-Chloro-8-fluoro-5-(2-((1-(3-((4-methoxybenzyl)amino)pyrazin-2-yl)ethyl)amino)ethoxy)-2-(methylthio)pyrido[4,3-d]pyrimidin-4(3H)-one

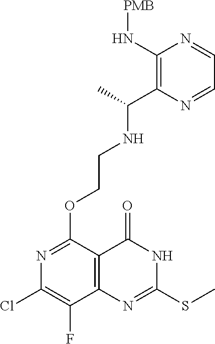

Sodium hydride (2.9 g, 72.5 mmol, 60% in mineral oil) was added to a solution of 2-[[(1R)-1-[3-[(4-methoxyphenyl)methylamino]pyrazin-2-yl]ethyl]amino]ethanol (6.5 g, 21.4 mmol) in tetrahydrofuran (50.0 mL) under nitrogen atmosphere at 0° C., and the mixture was stirred at 0° C. for 0.5 hour. Then the mixture was added to a solution of 5,7-dichloro-8-fluoro-2-methylsulfanyl-3H-pyrido[4,3-d]pyrimidin-4-one (5.0 g, 17.8 mmol) in tetrahydrofuran (50.0 mL) and stirred at room temperature for 1 hour. After completion, the reaction was quenched with sat. ammonium chloride aq. and extracted by ethyl acetate. The organic layer was washed by brine and dried over anhydrous sodium sulfate. After concentration under vacuum, the residue was used in next step without purification. LC-MS: (ESI, m/Z): 546.2 [M+H]⁺

### Step 2: (R)-3-(1-(5-Chloro-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrazin-2-amine

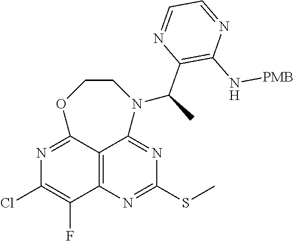

A mixture of 7-chloro-8-fluoro-5-[2-[[(1R)-1-[3-[(4-methoxyphenyl)methylamino]pyrazin-2-yl]ethyl]amino]ethoxy]-2-methylsulfanyl-3H-pyrido[4,3-d]pyrimidin-4-one (9.8 g, crude), bis(2-oxo-3-oxazolidinyl)phosphinic chloride (6.8 g, 26.9 mmol) and N,N-diisopropylethylamine (9.4 mL, 53.8 mmol) in chloroform (80 mL) was stirred at 70° C. for 1 hour. After completion, the reaction mixture was diluted with water and extracted with dichloromethane. The combined organic layers were washed with brine, dried over anhydrous sodium sulfate and concentrated in vacuum. The residue was purified by flash chromatography on silica gel eluting with petroleum ether/ethyl acetate (4:1) to afford the titled compound (7.0 g, 13.3 mmol, 74% yield) as a light yellow solid. LC-MS: (ESI, m/Z): 528.0 [M+H]⁺

### Step 3: (R)-3-(1-(5-(5-(Bis(4-methoxybenzyl)amino)-4-fluoro-3-methyl-2-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrazin-2-amine

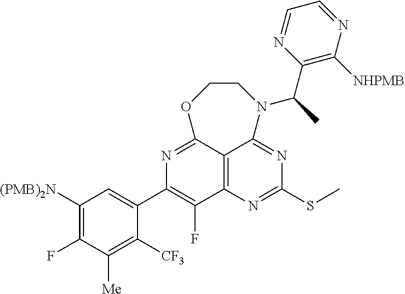

A mixture of (R)-3-(1-(5-chloro-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrazin-2-amine (350.0 mg, 0.66 mmol), [5-[bis[(4-methoxyphenyl)methyl]amino]-4-fluoro-3-methyl-2-(trifluoromethyl)phenyl]boronic acid (474.0 mg, 0.99 mmol), tetrakis(triphenylphosphine)palladium (77 mg, 0.07 mmol) and cesium carbonate (652 mg, 1.99 mmol) in 1,4-dioxane (5.0 mL) and water (0.2 mL) was stirred at 100° C. for 1 hour. After completion, the reaction mixture was diluted with water and extracted with dichloromethane. The combined organic layers were washed with brine, dried over sodium sulfate and concentrated in vacuum. The residue was purified by flash chromatography on silica gel eluting with dichloromethane/methanol (98:2) to afford the titled compound (480.0 mg, 0.52 mmol, 79% yield) as a light yellow solid. LC-MS: (ESI, m/Z): 925.5 [M+H]⁺

### Step 4: (R)-3-(1-(5-(5-(Bis(4-methoxybenzyl)amino)-4-fluoro-3-methyl-2-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N-(4-methoxybenzyl)pyrazin-2-amine

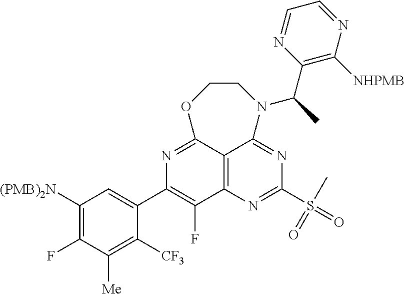

3-Chloroperoxybenzoic acid (176.0 mg, 0.86 mmol, 85% purity) was added to a solution of 3-[(1R)-1-[7-[5-[bis[(4-methoxyphenyl)methyl]amino]-4-fluoro-3-methyl-2-(trifluoromethyl)phenyl]-6-fluoro-3-methylsulfanyl-10-oxa-2,4,8,13-tetrazatricyclo[7.4.1.05, 14]tetradeca-1,3,5(14),6,8-pentaen-13-yl]ethyl]-N-[(4-methoxyphenyl)methyl]pyrazin-2-amine (200.0 mg, 0.22 mmol) in dichloromethane (10.0 mL) and stirred at room temperature for 1 hour. After completion, the reaction was quenched with sat. sodium sulfite aq. and extracted by dichloromethane. The organic layer was washed by brine and dried over anhydrous sodium sulfate. After concentration under vacuum, the residue was purified by flash chromatography on silica gel eluting with dichloromethane/methanol (97:3) to afford the titled compound (180.0 mg, 0.19 mmol, 87% yield) as a light yellow oil. LC-MS: (ESI, m/Z): 957.4 [M+H]⁺

### Step 5: 3-((R)-1-(5-(5-(Bis(4-methoxybenzyl)amino)-4-fluoro-3-methyl-2-(trifluoromethyl)phenyl)-2-(((S)-4,4-difluoro-1-methylpyrrolidin-2-yl)methoxy)-4-fluoro-8,9-dihydro-10H-7-oxa-1,3,6,10-tetraazacyclohepta[de]naphthalen-10-yl)ethyl)-N-(4-methoxybenzyl)pyrazin-2-amine

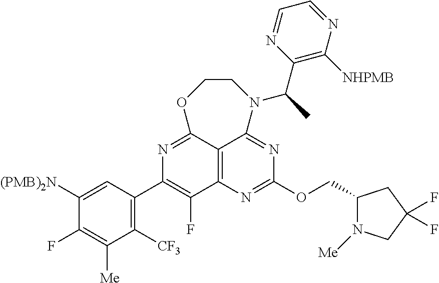

Sodium tert-butoxide (72 mg, 0.75 mmol) was added to a solution of [(2S)-4,4-difluoro-1-methyl-pyrrolidin-2-yl]methanol (85 mg, 0.56 mmol) in toluene (3.0 mL) under nitrogen atmosphere at 0° C., and the mixture was stirred at 0° C. for 0.5 hour. Then the mixture was added to a solution of 3-[(1R)-1-[7-[5-[bis[(4-methoxyphenyl)methyl]amino]-4-fluoro-3-methyl-2-(trifluoromethyl)phenyl]-6-fluoro-3-methylsulfonyl-10-oxa-2,4,8,13-tetrazatricyclo[7.4.1.05, 14]tetradeca-1,3,5(14),6,8-pentaen-13-yl]ethyl]-N-[(4-methoxyphenyl)methyl]pyrazin-2-amine (180.0 mg, 0.19 mmol) in toluene (3.0 mL) and stirred at room temperature for 1 hour. After completion, the reaction was quenched with sat. ammonium chloride aq. and extracted by ethyl acetate. The organic layer was washed by brine and dried over anhydrous sodium sulfate. After concentration under vacuum, the residue was purified by flash chromatography on silica gel eluting with dichloromethane/methanol (97:3) to afford the titled compound (130.0 mg, 0.13 mmol, 67% yield) as a light yellow solid. LC-MS: (ESI, m/Z): 1028.4 [M+H]⁺

### Step 6: 3-((R)-1-(5-(5-Amino-4-fluoro-3-methyl-2-(trifluoromethyl)phenyl)-2-(((S)-4,4-difluoro-1-methylpyrrolidin-2-yl)methoxy)-4-fluoro-8,9-dihydro-10H-7-oxa-1,3,6,10-tetraazacyclohepta[de]naphthalen-10-yl)ethyl)pyrazin-2-amine

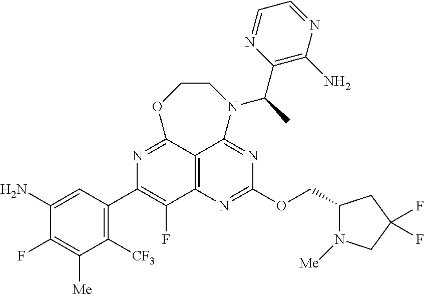 [SMILES: Cc1c(F)c(N)cc(-c2nc3c4c(nc(OC[C@@H]5CC(F)(F)CN5C)nc4c2F)N([C@H](C)c2nccnc2N)CCO3)c1C(F)(F)F]

A mixture of 3-[(1R)-1-[7-[5-[bis[(4-methoxyphenyl)methyl]amino]-4-fluoro-3-methyl-2-(trifluoromethyl)phenyl]-3-[[(2S)-4,4-difluoro-1-methyl-pyrrolidin-2-yl]methoxy]-6-fluoro-10-oxa-2,4,8,13-tetrazatricyclo[7.4.1.05, 14]tetradeca-1,3,5(14),6,8-pentaen-13-yl]ethyl]-N-[(4-methoxyphenyl)methyl]pyrazin-2-amine (125.0 mg, 0.12 mmol) in trifluoroacetic acid (5.0 mL) and trifluoromethanesulfonic acid (0.5 mL) was stirred at room temperature for 1 hour. The reaction mixture was diluted with dichloromethane, basified with sat. sodium bicarbonate aq. to adjust pH to 8, extracted with dichloromethane, dried over sodium sulfate and concentrated in vacuum. The crude product was purified by Prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 35% B to 55% B in 11 min; Wave Length: 254 nm; RT1(min): 10.8; to afford the titled compound (23.8 mg, 0.04 mmol, 29% yield).

Example 8: ¹H NMR (300 MHz, DMSO-d₆, ppm) δ 7.95 (d, J=2.7 Hz, 1H), 7.78 (d, J=2.7 Hz, 1H), 6.55 (d, J=8.6 Hz, 1H), 6.44-6.24 (m, 3H), 6.00 (s, 2H), 4.65-4.26 (m, 4H), 3.88 (dd, J=15.7, 6.4 Hz, 1H), 3.79-3.60 (m, 1H), 3.50-3.36 (m, 1H), 3.03-2.86 (m, 1H), 2.78-2.56 (m, 1H), 2.48-2.39 (m, 1H), 2.37 (s, 3H), 2.32 (s, 3H), 2.28-2.05 (m, 1H), 1.57 (d, J=6.8 Hz, 3H). LC-MS: (ESI, m/Z): 668.2 [M+H]⁺

### Example 9: 3-((R)-1-(5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine

### Synthetic Route

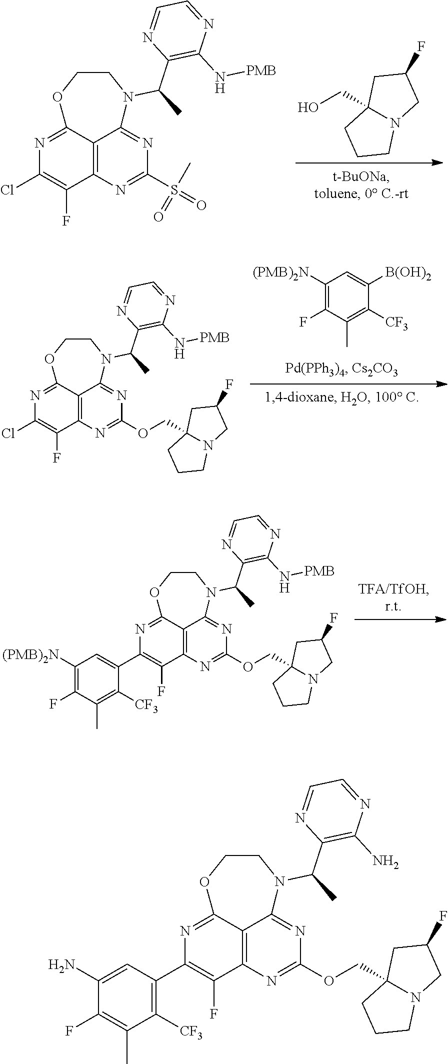

### Step 1: 3-((R)-1-(5-Chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrazin-2-amine

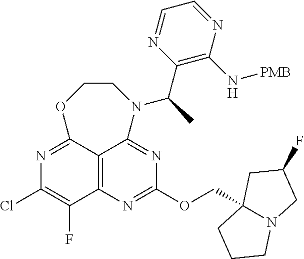

Sodium tert-butoxide (350.0 mg, 3.6 mmol) was added to a solution of ((2R,7aS)-2-fluorotetrahydro-1H-pyrrolizin-7a(5H)-yl)methanol (435.0 mg, 2.7 mmol) in toluene (15.0 mL) under nitrogen atmosphere at 0° C., and the mixture was stirred at 0° C. for 0.5 hour. Then the mixture was added to a solution of 3-[(1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl]-N-[(4-methoxyphenyl)methyl]pyrazin-2-amine (510.0 mg, 0.9 mmol) in toluene (15.0 mL) and stirred at room temperature for 1 hour. After completion, the reaction was quenched with sat. ammonium chloride aq. and extracted by ethyl acetate. The organic layer was washed by brine and dried over anhydrous sodium sulfate. After concentration under vacuum, the residue was purified by flash chromatography on silica gel eluting with dichloromethane/methanol (97:3) to afford the titled compound (195.0 mg, 0.3 mmol, 34% yield) as a light yellow solid. LC-MS: (ESI, m/Z): 639.2 [M+H]⁺

### Step 2: 3-((R)-1-(5-(5-(Bis(4-methoxybenzyl)amino)-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrazin-2-amine

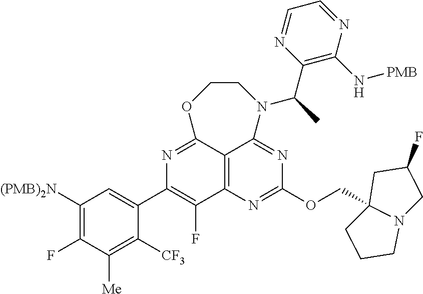

A mixture of 3-((R)-1-(5-Chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrazin-2-amine (195.0 mg, 0.3 mmol), [5-[bis[(4-methoxyphenyl)methyl]amino]-4-fluoro-3-methyl-2-(trifluoromethyl)phenyl]boronic acid (218.0 mg, 0.5 mmol), tetrakis(triphenylphosphine)palladium (35 mg, 0.03 mmol) and cesium carbonate (300.0 mg, 0.9 mmol) in 1,4-dioxane (6.0 mL) and water (0.6 mL) was stirred at 100° C. for 1 hour. After completion, the reaction mixture was diluted with water and extracted with dichloromethane. The combined organic layers were washed with brine, dried over anhydrous sodium sulfate and concentrated in vacuum. The residue was purified by flash chromatography on silica gel eluting with dichloromethane/methanol (49:1) to afford the titled compound (300.0 mg, 0.3 mmol, 95% yield) as a light yellow solid. LC-MS: (ESI, m/Z): 1036.6 [M+H]⁺

### Step 3: 3-((R)-1-(5-(5-Amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine

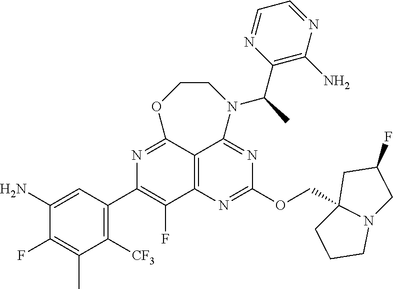 [SMILES: Cc1c(F)c(N)cc(-c2nc3c4c(nc(OC[C@@]56CCCN5C[C@H](F)C6)nc4c2F)N([C@H](C)c2nccnc2N)CCO3)c1C(F)(F)F]

A mixture of 3-((R)-1-(5-(5-(Bis(4-methoxybenzyl)amino)-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrazin-2-amine (300.0 mg, 0.3 mmol) in trifluoroacetic acid (6.0 mL) and trifluoromethanesulfonic acid (0.6 mL) was stirred at room temperature for 1 hour. The reaction mixture was diluted with dichloromethane and basified with sat. sodium bicarbonate aq. to adjust pH to 8, extracted with dichloromethane, dried over sodium sulfate and concentrated in vacuum. The residue was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 29% B to 56% B in 9.5 min, 56% B; Wave Length: 254/220 nm; RT1(min): 8.33) to afford the titled compound (31.8 mg, 0.047 mmol, 16% yield).

Example 9: ¹H NMR (300 MHz, DMSO-d₆, ppm) δ 7.94 (d, J=2.7 Hz, 1H), 7.78 (d, J=2.7 Hz, 1H), 6.55 (d, J=8.7 Hz, 1H), 6.37 (d, J=6.8 Hz, 3H), 6.00 (s, 2H), 5.27 (d, J=56.1, Hz, 1H), 4.64-4.45 (m, 1H), 4.39 (dd, J=12.8, 6.4 Hz, 1H), 4.21-3.99 (m, 2H), 3.89 (dd, J=15.9, 6.5 Hz, 1H), 3.78-3.55 (m, 1H), 3.17-3.02 (m, 2H), 3.05-2.92 (m, 1H), 2.92-2.69 (m, 1H), 2.44-2.26 (m, 3H), 2.21-2.08 (m, 1H), 2.00 (s, 2H), 1.89-1.67 (m, 3H), 1.56 (d, J=6.8 Hz, 3H). LC-MS: (ESI, m/Z): 676.2 [M+H]⁺

### Example 10: 3-((R)-1-(5-(5-amino-4-fluoro-3-methyl-2-(trifluoromethyl)phenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyrazin-2-amine

### Synthetic Route

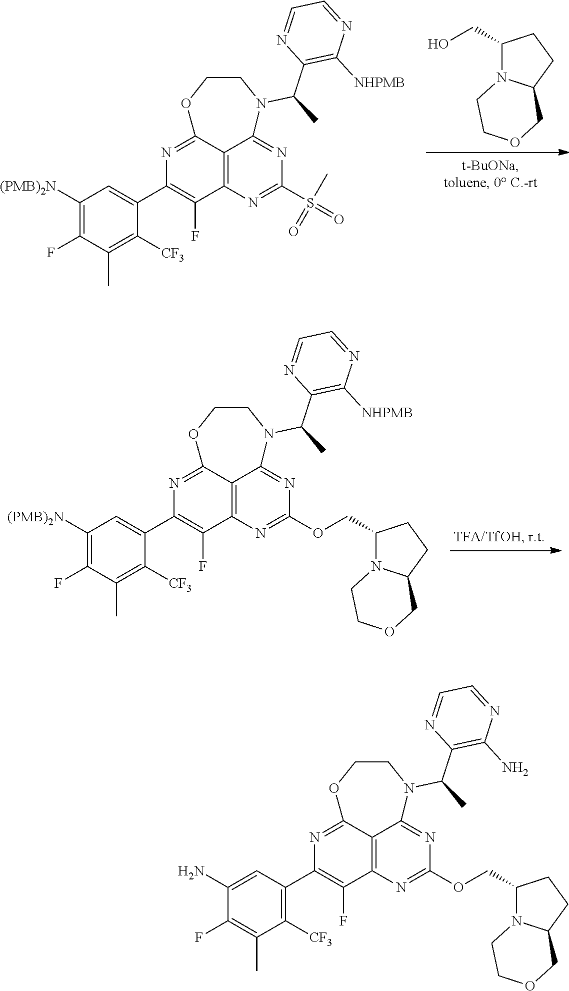

### Step 1: 3-((R)-1-(5-(5-(Bis(4-methoxybenzyl)amino)-4-fluoro-3-methyl-2-(trifluoromethyl)phenyl)-4-fluoro-2-(((6S,8aS)-hexahydro-1H-pyrrolo[2, 1-c][1,4]oxazin-6-yl)methoxy)-8,9-dihydro-10H-7-oxa-1,3,6,10-tetraazacyclohepta[de]naphthalen-10-yl)ethyl)-N-(4-methoxybenzyl)pyrazin-2-amine

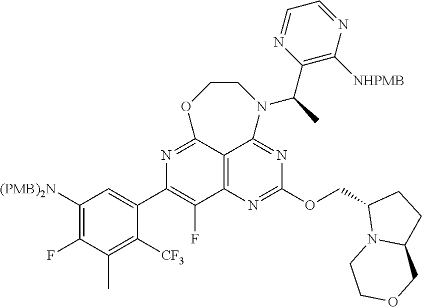

Sodium tert-butoxide (72.0 mg, 0.75 mmol) was added to a solution of ((6S,8aS)-hexahydro-1H-pyrrolo[2,1-c][1,4]oxazin-6-yl)methanol (98.0 mg, 0.63 mmol) in toluene (3.0 mL) under nitrogen atmosphere at 0° C., and the mixture was stirred at 0° C. for 0.5 hour. Then the mixture was added to a solution of 3-[(1R)-1-[7-[5-[bis[(4-methoxyphenyl)methyl]amino]-4-fluoro-3-methyl-2-(trifluoromethyl)phenyl]-6-fluoro-3-methylsulfonyl-10-oxa-2,4,8,13-tetrazatricyclo[7.4.1.05, 14]tetradeca-1,3,5(14),6,8-pentaen-13-yl]ethyl]-N-[(4-methoxyphenyl)methyl]pyrazin-2-amine (200.0 mg, 0.21 mmol) in toluene (3.0 mL) and stirred at room temperature for 1 hour. After completion, the reaction was quenched with sat. ammonium chloride aq. and extracted by ethyl acetate. The organic layer was washed by brine and dried over anhydrous sodium sulfate. After concentration under vacuum, the residue was purified by flash chromatography on silica gel eluting with dichloromethane/methanol (97:3) to afford the titled compound (130.0 mg, 0.13 mmol, 60% yield) as a light yellow solid. LC-MS: (ESI, m/Z): 1034.5 [M+H]⁺

### Step 2: 3-((R)-1-(5-(5-Amino-4-fluoro-3-methyl-2-(trifluoromethyl)phenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyrazin-2-amine

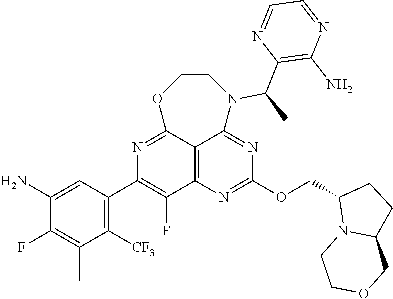 [SMILES: Cc1c(F)c(N)cc(-c2nc3c4c(nc(OC[C@@H]5CC[C@H]6COCCN65)nc4c2F)N([C@H](C)c2nccnc2N)CCO3)c1C(F)(F)F]

A mixture of 3-((R)-1-(5-(5-(bis(4-methoxybenzyl)amino)-4-fluoro-3-methyl-2-(trifluoromethyl)phenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)-N-(4-methoxybenzyl)pyrazin-2-amine (130.0 mg, 0.13 mmol), trifluoroacetic acid (4.0 mL) and trifluoromethanesulfonic acid (0.4 mL) was stirred at room temperature for 1 hour. After completion, the reaction mixture was diluted with dichloromethane and basified with sat. sodium bicarbonate aq. to adjust pH to 10. Then extracted with dichloromethane and the combined organic layers were washed with brine, dried over sodium sulfate and concentrated in vacuum. The crude product was purified by Prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 29% B to 49% B in 10 min; Wave Length: 254 nm; RT1(min): 10.2) to afford the titled compound (38.0 mg, 0.06 mmol, 45% yield, 99.1% purity).

Example 10: ¹H NMR (300 MHz, DMSO-d₆, ppm) 67.94 (d, J=2.7 Hz, 1H), 7.78 (d, J=2.7 Hz, 1H), 6.55 (d, J=8.6 Hz, 1H), 6.46-6.24 (m, 3H), 6.00 (s, 2H), 4.69-4.13 (m, 4H), 3.88 (dd, J=15.9, 6.4 Hz, 1H), 3.77-3.58 (m, 1H), 3.58-3.47 (m, 1H), 3.48-3.38 (m, 3H), 3.13 (t, J=10.3 Hz, 1H), 3.03-2.74 (m, 3H), 2.32 (dd, J=4.4, 2.1Hz, 3H), 2.17-1.96 (m, 1H), 1.87-1.68 (m, 1H), 1.69-1.49 (m, 4H), 1.43-1.19 (m, 1H). LC-MS: (ESI, m/Z): 674.2 [M+H]⁺

### Example 11: 4-(10-((R)-1-(3-aminopyrazin-2-yl) ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-ethynyinaphthalen-2-ol

### Synthetic Route

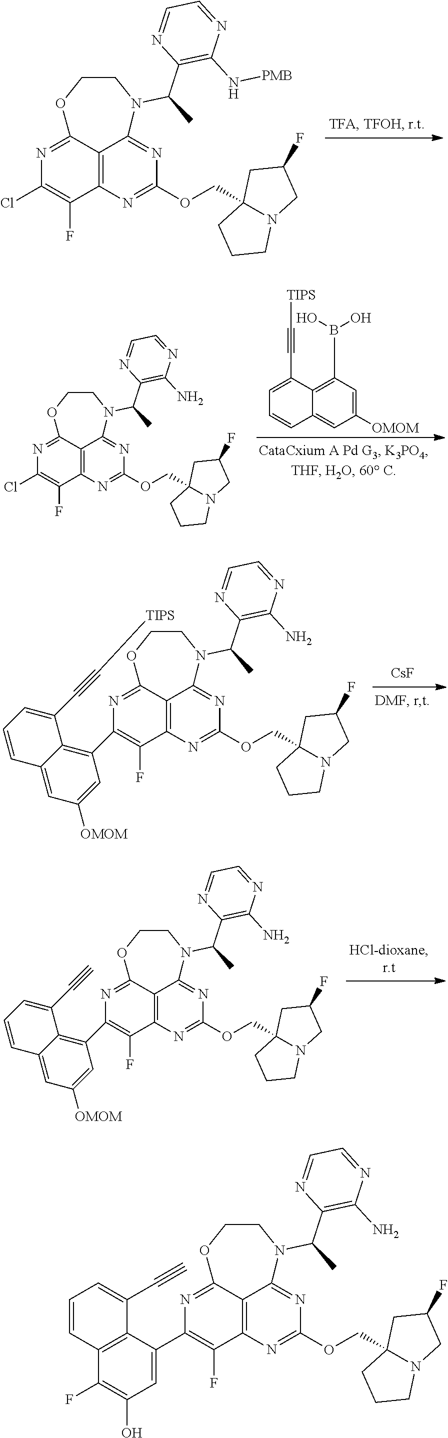

### Step 1: 3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine

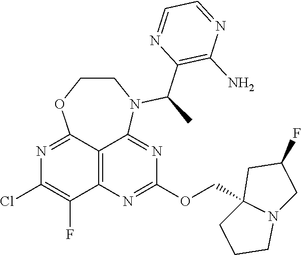 [SMILES: C[C@H](c1nccnc1N)N1CCOc2nc(Cl)c(F)c3nc(OC[C@@]45CCCN4C[C@H](F)C5)nc1c23]

A solution of 3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrazin-2-amine (644.7 mg, 0.4 mmol) in 2,2,2-trifluoroacetic acid (5.0 mL) and trifluoromethanesulfonic acid (0.5 mL) was stirred at 25° C. for 10 minutes. After completion, the solvent was concentrated under vacuum. The reaction mixture was diluted with dichloromethane and adjusted to pH=8 with sat. sodium carbonate aqueous solution. The solution was washed with water, dried over anhydrous sodium sulfate and concentrated under vacuum. The residue was purified by flash chromatography on silica gel eluting with dichloromethane/methanol (10:1) to afford the title compound (277.1 mg, 0.5 mmol, 52.6% yield) as a yellow solid. LC-MS: (ESI, m/Z): 519.2 M+H]⁺

### Step 2: 3-((R)-1-(4-fluoro-2-(((2R,7aS)-2-fluorotetrahydro-1H-pyrrolizin-7a(5H)-yl)methoxy)-5-(3-(methoxymethoxy)-8-((triisopropylsilyl)ethynyl)naphthalen-1-yl)-8,9-dihydro-10H-7-oxa-1,3,6,10-tetraazacyclohepta[de]naphthalen-10-yl)ethyl)pyrazin-2-amine

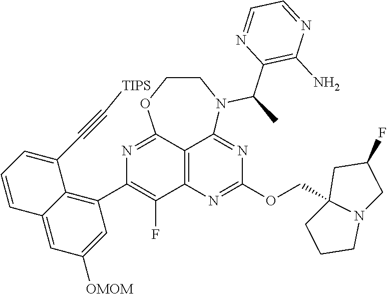

Under nitrogen, a solution of 3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine (277.1 mg, 0.5 mmol), (3-(methoxymethoxy)-8-((triisopropylsilyl)ethynyl)naphthalen-1-yl)boronic acid (880.8 mg, 2.1 mmol), [(di(1-adamantyl)-butylphosphine)-2-(2′-amino-1,1′-biphenyl)]palladium(II) methanesulfonate (155.4 mg, 0.2 mmol) and potassium phosphate tribasic (452.8 mg, 2.1 mmol) in tetrahydrofuran (3.0 mL) and water (0.6 mL) was added at 25° C. The resulting solution was stirred for 1 hour at 60° C. After completion, the resulting solution was diluted with dichloromethane and washed with water. The organic layer was dried over anhydrous sodium sulfate and concentrated under vacuum. The residue was purified by flash chromatography on silica gel eluting with dichloromethane/methanol (10:1) to afford the title compound (365.1 mg, 0.3 mmol, 71.9% yield) as a yellow solid. LC-MS: (ESI, m/Z): 851.4 [M+H]⁺

### Step 3: 3-((R)-1-(5-(8-ethynyl-3-(methoxymethoxy)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine

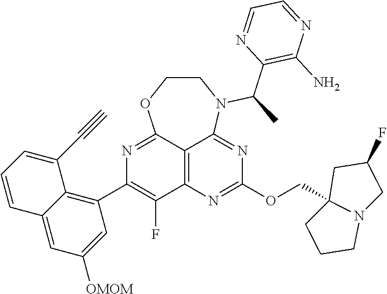

A solution of 3-((R)-1-(4-fluoro-2-(((2R,7aS)-2-fluorotetrahydro-1H-pyrrolizin-7a(5H)-yl)methoxy)-5-(3-(methoxymethoxy)-8-((triisopropylsilyl)ethynyl)naphthalen-1-yl)-8,9-dihydro-10H-7-oxa-1,3,6,10-tetraazacyclohepta[de]naphthalen-10-yl)ethyl)pyrazin-2-amine (365.1 mg, 0.3 mmol) and caesium fluoride (184.5 mg, 1.2 mmol) in N,N-dimethylformamide (4.0 mL) was stirred at 25° C. for 1 hour. After completion, the reaction mixture was diluted with dichloromethane, washed with water, dried over anhydrous sodium sulfate and concentrated under vacuum. The residue was purified by flash chromatography on silica gel eluting with dichloromethane/ethyl acetate (1:1) to afford the title compound (244.4 mg, 0.3 mmol, 82.5% yield) as a yellow solid. LC-MS: (ESI, m/Z): 695.3 [M+H]⁺

### Step 4: 4-(10-((R)-1-(3-aminopyrazin-2-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-ethynylnaphthalen-2-ol

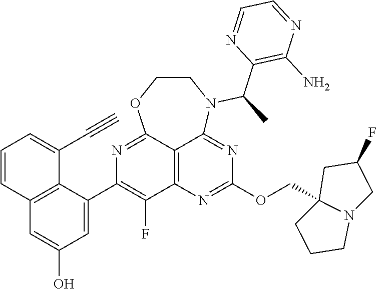 [SMILES: C#Cc1cccc2cc(O)cc(-c3nc4c5c(nc(OC[C@@]67CCCN6C[C@H](F)C7)nc5c3F)N([C@H](C)c3nccnc3N)CCO4)c12]

A solution of 3-((R)-1-(5-(8-ethynyl-3-(methoxymethoxy)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine (50.0 mg, 0.07 mmol) and hydrochloric acid/1,4-dioxane (1 mL,4.0 M in 1,4-dioxane) in acetonitrile (0.5 mL) was stirred at 25° C. for 30 minutes. After completion, the solvent was concentrated under vacuum. The residue was dissolved with dichloromethane and the pH was adjusted to 8 with saturated sodium bicarbonate aqueous solution. The resulting solution was washed with water. The organic layer was dried over anhydrous sodium sulfate and concentrated under vacuum. The residue was purified by flash chromatography on silica gel eluting with dichloromethane/methanol (10:1) to afford product as a yellow solid. The product was further purified by prep-HPLC by following conditions: Column: Xselect CSH C18 OBD Column 30*150 mm 5 μm; Mobile Phase A: Water(0.1% FA), Mobile Phase B: ACN; Flow rate: 60 mL/min; Gradient: 12% B to 30% B in 9 min, 30% B; Wave Length: 254/220 nm; RT1(min): 9 to afford the title compound (4.6 mg, 0.007 mmol, 9.8% yield). LC-MS: (ESI, m/Z): 651.2 [M+H]⁺

Example 11: ¹H NMR (300 MHz, Methanol-d₄, ppm) δ 7.93 (dd, J=2.8, 1.4 Hz, 1H), 7.85 (d, J=2.7 Hz, 1H), 7.80 (d, J=8.2 Hz, 1H), 7.55-7.46 (m, 1H), 7.45-7.33 (m, 1H), 7.30 (d, J=2.6 Hz, 1H), 7.14 (dd, J=6.4, 2.6 Hz, 1H), 6.51 (q, J=6.9 Hz, 1H), 5.40 (d, J=52.7 Hz, 1H), 4.73-4.31 (m, 4H), 4.17-3.84 (m, 2H), 3.70-3.42 (m, 3H), 3.26-3.03 (m, 2H), 2.58-1.88 (m, 6H), 1.71 (dd, J=6.9, 2.5 Hz, 3H). LC-MS: (ESI, m/Z): 651.3 [M+H]⁺

### Example 12: 3-((1R)-1-(5-(3-amino-2,4-difluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Synthetic Route

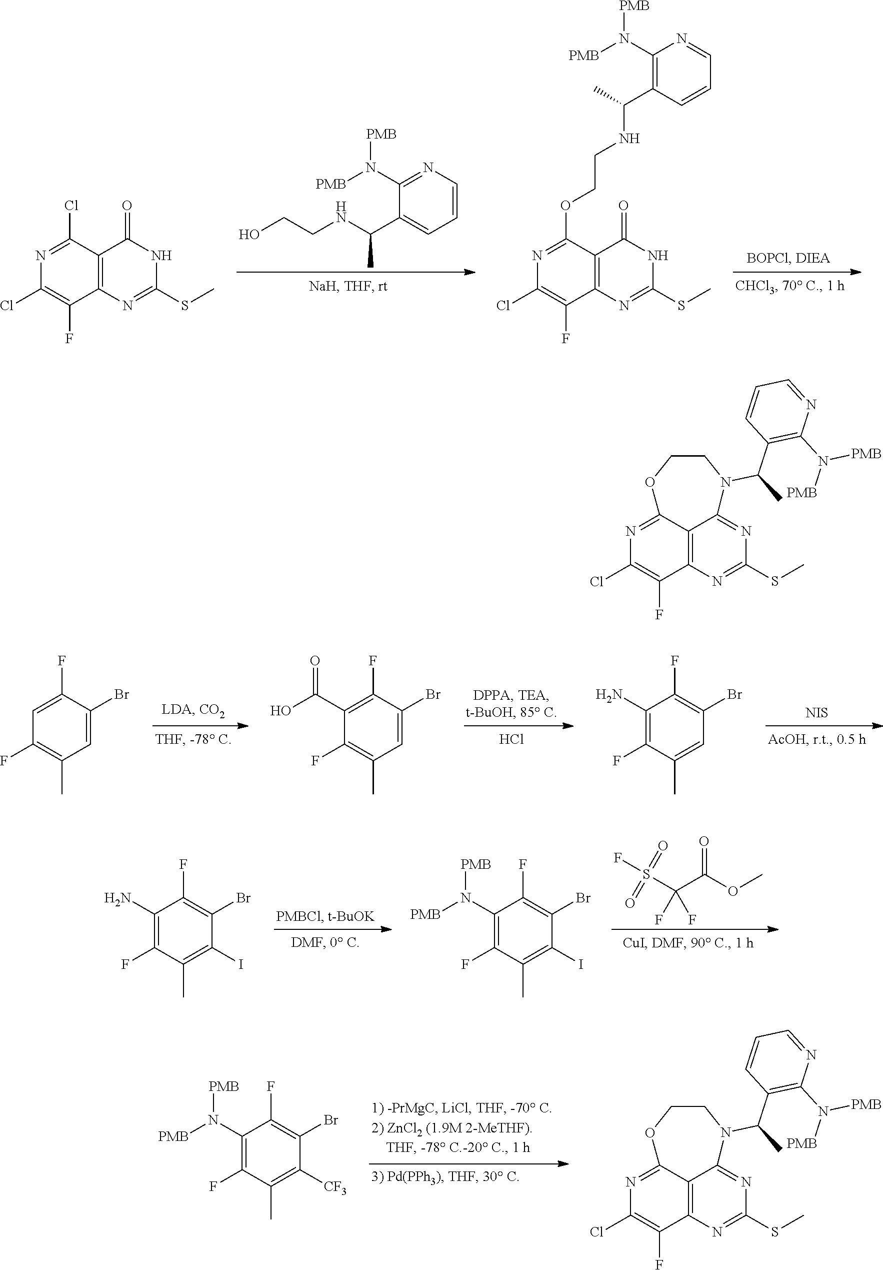

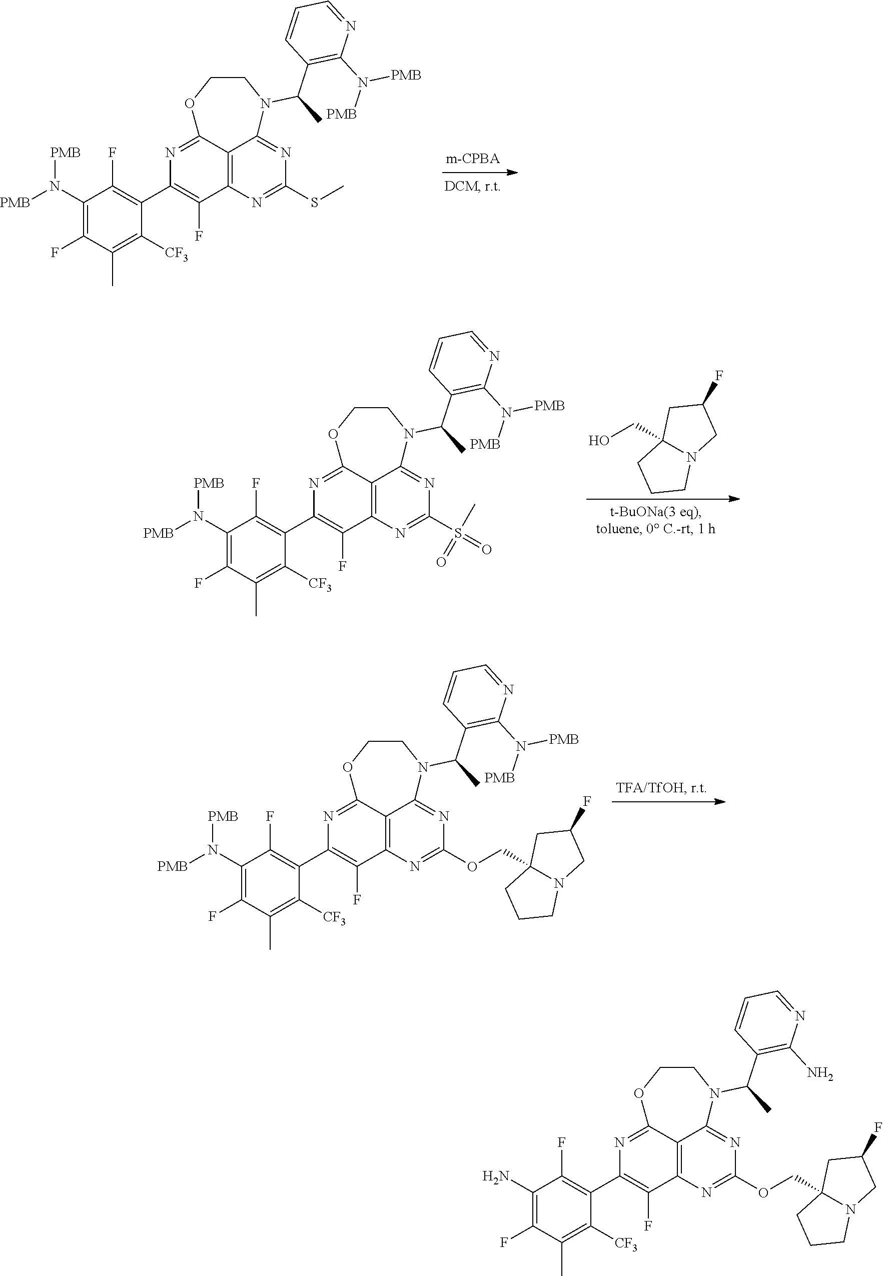

Step 1: (R)-5-(2-((1-(2-(bis(4-methoxybenzyl)amino)pyridin-3-yl)ethyl)amino)ethoxy)-7-chloro-8-fluoro-2-(methylthio)pyrido[4, 3-d]pyrimidin-4(3H)-one

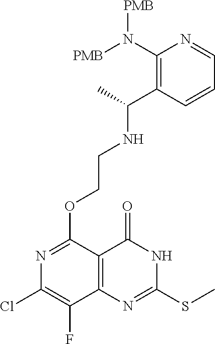

A solution of 2-[[(1R)-1-[2-[bis[(4-methoxyphenyl)methyl]amino]-3-pyridyl]ethyl]amino]ethanol (3.31 g, 7.8 mmol) in tetrahydrofuran (20 mL) was added sodium hydride (1.14 g, 28.5 mmol, 60% purity) and stirred for 10 min at room temperature. Then the reaction mixture was added into a solution of 5,7-dichloro-8-fluoro-2-methylsulfanyl-3H-pyrido[4,3-d]pyrimidin-4-one (2.00 g, 7.1 mmol) tetrahydrofuran (20 mL) and stirred for 1 hour. After completion, the reaction was quenched with saturated ammonium chloride solution. The resulting solution was extracted with ethyl acetate, washed with brine, dried over anhydrous sodium sulfate and concentrated. The residue was purified by flash chromatography on silica gel eluting with dichloromethane/methanol (10:1) to afford the title compound (2.4 g, 3.6 mmol, 50.5% yield) as a white solid. LC-MS: (ESI, m/Z): 665.3 [M+H]⁺

### Step 2: (R)-3-(1-(5-chloro-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine

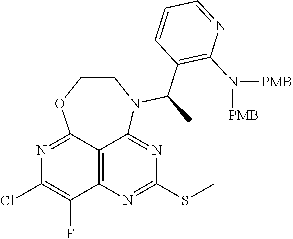

A solution of (R)-5-(2-((1-(2-(bis(4-methoxybenzyl)amino)pyridin-3-yl)ethyl)amino)ethoxy)-7-chloro-8-fluoro-2-(methylthio)pyrido[4,3-d]pyrimidin-4(3H)-one (3.6 g, 5.41 mmol) and N,N-diisopropylethylamine (4.7 mL, 27.0 mmol) in chloroform (40 mL) was added bis(2-oxo-3-oxazolidinyl) phosphinicchloride (2.1 g, 8.1 mmol) and stirred at 70° C. for 1 hour. After completion, the reaction was diluted with water, extracted with ethyl acetate, over anhydrous sodium sulfate and concentrated. The residue was purified by flash chromatography on silica gel eluting with petroleum ether/ethyl acetate (3:2) to afford the title compound (1.37 g, 1.9 mmol, 36.2% yield) as a white solid. LC-MS: (ESI, m/Z): 647.5 [M+H]⁺

### Step 3: 3-bromo-2,6-difluoro-5-methylbenzoic acid

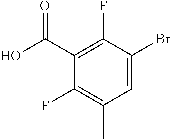

Under nitrogen, a solution of 1-bromo-2,4-difluoro-5-methyl-benzene (10.0 g, 48.3 mmol) in tetrahydrofuran (100 mL) was slowly added lithium diisopropylamide (31.5 mL, 63.0 mmol) (2M in tetrahydrofuran) at −78° C. and stirred for 2 hours at −78° C. Then dry ice(s) was poured into the reaction solution and stirred at room temperature for 1 hour. After completion, the reaction solution was quenched with saturated ammonium chloride solution and adjusted to pH=7 with 1 N hydrochloric acid. Then the reaction mixture was diluted with water. The resulting solution was extracted with ethyl acetate and the organic layers were combined. The organic layer was dried over anhydrous sodium sulfate and concentrated under vacuum to afford 3-bromo-2,6-difluoro-5-methyl-benzoic acid (9.00 g, curde) as a light yellow solid. The crude product was used in the next step directly. LC-MS: (ESI, m/Z): 251.3 [M−H]⁺

### Step 4: 3-bromo-2,6-difluoro-5-methylaniline

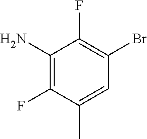

A solution of 3-bromo-2,6-difluoro-5-methyl-benzoic acid (5.80 g, 19.9 mmol), diphenylphosphonicazide (12.70 g, 39.8 mmol) and triethylamine (7.0 g, 59.7 mmol) in tert-butanol (50.0 mL) was stirred at 85° C. for 1.5 hours. Then the solution was added 2N hydrogen chloride (10 mL) and stirred for 1 hour. After completion, the reaction mixture was quenched with sat. sodium carbonate aq. The resulting solution was extracted with ethyl acetate, washed with water, dried over anhydrous sodium sulfate and concentrated under vacuum to afford the title compound (2.50 g, crude) as a yellow soil. The crude product was used in the next step directly. LC-MS: (ESI, m/Z): 222.4 [M+H]⁺

### Step 5: 3-bromo-2,6-difluoro-4-iodo-5-methylaniline

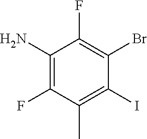

A solution of 3-bromo-2,6-difluoro-5-methyl-aniline (1.0 g, 4.5 mmol) in acetic acid (10 mL) was added N-iodosuccinimide (1.22 g, 5.4 mmol) and stirred at 25° C. for 1 hour. After completion, the reaction mixture was concentrated under reduced pressure. Then the reaction was quenched with saturated sodium bicarbonate solution. Then the reaction mixture was diluted with water. The resulting solution was extracted with ethyl acetate, washed with water, dried over anhydrous sodium sulfate and concentrated under vacuum. The residue was purified by flash chromatography on silica gel eluting with methanol/dichloromethane (1:8) to afford the title compound (1.10 g, 3.1 mmol) as a white solid. LC-MS: (ESI, m/Z): 348.2 [M+H]⁺

### Step 6: 3-bromo-2,6-difluoro-4-iodo-N,N-bis(4-methoxybenzyl)-5-methylaniline

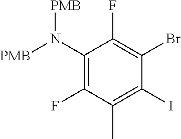

A solution of 3-bromo-2,6-difluoro-4-iodo-5-methyl-aniline (1.0 g, 2.87 mmol) and potassium tert-butoxide (1.29 g, 11.5 mmol) in N,N-dimethylformamide (10 mL) was added p-methoxybenzyl chloride (1.3 g, 8.6 mmol) and stirred at 0° C. for 1.5 hours. After completion, the reaction mixture was diluted with water, extracted with ethyl acetate, washed with water, dried over anhydrous sodium sulfate and concentrated under vacuum. The residue was purified by flash chromatography on silica gel eluting with ethyl acetate/petroleum ether (1:9) to afford the title compound (1.2 g, 2.0 mmol, 71% yield) as a white solid. LC-MS: (ESI, m/Z): 588.7 [M+H]⁺

### Step 7: 3-bromo-2,6-difluoro-N,N-bis(4-methoxybenzyl)-5-methyl-4-(trifluoromethyl)aniline

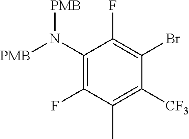

Under nitrogen, a solution of 3-bromo-2,6-difluoro-4-iodo-N,N-bis[(4-methoxyphenyl)methyl]-5-methyl-aniline (100.0 mg, 0.2 mmol) and cuprous iodide (323.0 mg, 1.7 mmol) in N,N-dimethylformamide (1 mL) was stirred at −40° C. for 10 minutes. Then methyl 2,2-difluoro-2-(fluorosulfonyl)acetate (0.2 mL, 1.7 mmol) was added at room temperature and stirred at 90° C. for 1 hour. After completion, the reaction mixture was diluted with water, extracted with ethyl acetate, washed with water and dried over anhydrous sodium sulfate and concentrated under vacuum. The residue was purified by flash chromatography on silica gel eluting with methanol/dichloromethane (1:8) to afford the title compound (80.0 mg, 0.15 mmol, 88.7% yield) as a white solid. LC-MS: (ESI, m/Z): 530.1 [M+H]⁺

### Step 8: 3-((1R)-1-(5-(3-(Bis(4-methoxybenzyl)amino)-2,4-difluoro-5-methyl-6-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine

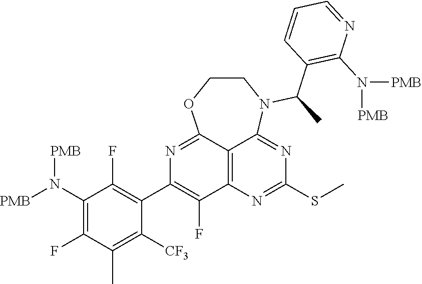

Isopropylmagnesium chloride. lithium chloride (1.3 M in tetrahydrofuran) (2.6 mL, 3.4 mmol) was added to a solution of 3-bromo-2,6-difluoro-N,N-bis[(4-methoxyphenyl)methyl]-5-methyl-4-(trifluoromethyl)aniline (1.20 g, 2.3 mmol) in tetrahydrofuran (13 mL) at −78° C. under nitrogen atmosphere. After sitrred at −78° C. for 30 min, zinc chloride (1.9 M 2-methyltetrahydrofuran) (3.4 mL, 6.79 mmol) was added to the mixture and stirred for 10 min. Then the mixture was warmed to room temperature and stirred at room temperature for 30 min. Then the reaction solution was added to a solution of 3-[(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl]-N,N-bis[(4-methoxyphenyl)methyl]pyridin-2-amine (1.40 g, 2.3 mmol) and tetrakis(triphenylphosphine)palladium (0.30 g, 0.2 mmol) in tetrahydrofuran (13 mL) under nitrogen and stirred at 80° C. 5h. After completion, the reaction was quenched with saturated ammonium chloride solution, diluted with water, extracted with ethyl acetate and the organic layers were combined. The organic layer was dried over anhydrous sodium sulfate and concentrated under vacuum. The residue was purified by flash chromatography on silica gel eluting with methanol/dichloromethane (1:8) to afford the titled compound (600.0 mg, 0.5 mmol, 25% yield) as a white solid. LC-MS: (ESI, m/Z): 1062.5 [M+H]⁺

### Step 9: 3-((1R)-1-(5-(3-(bis(4-methoxybenzyl)amino)-2,4-difluoro-5-methyl-6-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine

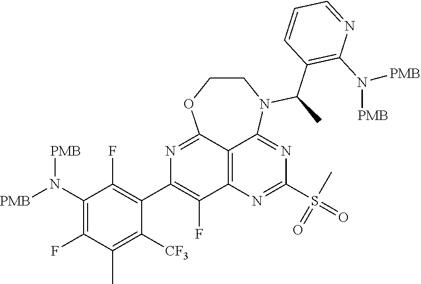

Under nitrogen, a solution of 3-[(1R)-1-[7-[3-[bis[(4-methoxyphenyl)methyl]amino]-2,4-difluoro-5-methyl-6-(trifluoromethyl)phenyl]-6-fluoro-3-methylsulfanyl-10-oxa-2,4,8,13-tetrazatricyclo[7.4.1.05, 14]tetradeca-1,3,5(14),6,8-pentaen-13-yl]ethyl]-N,N-bis[(4-methoxyphenyl)methyl]pyridin-2-amine (100.0 mg, 0.1 mmol) and 3-chloroperoxybenzoic acid (48.7 mg, 0.3 mmol) in dichloromethane (1 mL) was stirred at 25° C. for 1.5 hours. After completion, the reaction was quenched with saturated sodium sulfite solution. The resulting solution was extracted with ethyl acetate, washed with water dried over anhydrous sodium sulfate and concentrated under vacuum. The residue was purified by flash chromatography on silica gel eluting with methanol/dichloromethane (1:8) to afford the title compound (70.0 mg, 0.06 mmol, 68% yield) as a white solid. LC-MS: (ESI, m/Z): 1093.9 [M+H]⁺

### Step 10: 3-((1R)-1-(5-(3-(bis(4-methoxybenzyl)amino)-2,4-difluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine

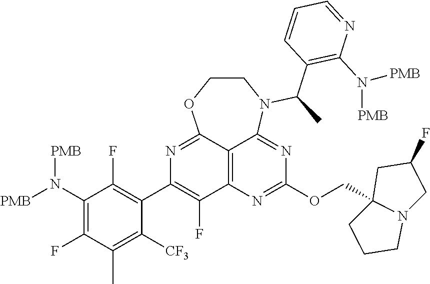

A solution of ((2R,7aS)-2-fluorotetrahydro-1H-pyrrolizin-7a(5H)-yl)methanol (43.6 mg, 0.3 mmol) in toluene (1 mL) was added sodium tert-butoxide (43.8 mg, 0.5 mmol) and stirred at 0° C. for 10 minutes. Then the reaction mixture was added 3-[(1R)-1-[7-[3-[bis[(4-methoxyphenyl)methyl]amino]-2,4-difluoro-5-methyl-6-(trifluoromethyl)phenyl]-6-fluoro-3-methylsulfonyl-10-oxa-2,4,8,13-tetrazatricyclo[7.4.1.05, 14]tetradeca-1,3,5(14),6,8-pentaen-13-yl]ethyl]-N,N-bis[(4-methoxyphenyl)methyl]pyridin-2-amine (100.0 mg, 0.1 mmol) and stirred at 25° C. for 1.5 hours. After completion, the reaction was quenched with saturated ammonium chloride solution. The reaction mixture was diluted with water, extracted with ethyl acetate, dried over anhydrous sodium sulfate and concentrated under vacuum. The residue was purified by flash chromatography on silica gel eluting with ethyl acetate/petroleum ether (1:8) to afford the title compound (90.0 mg, 0.07 mmol, 83.9% yield) as a yellow solid. LC-MS: (ESI, m/Z): 1173.6 [M+H]⁺

### Step 11: 3-((1R)-1-(5-(3-amino-2,4-difluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

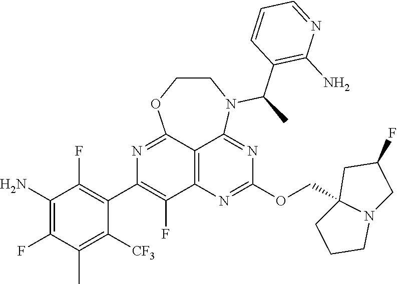 [SMILES: Cc1c(F)c(N)c(F)c(-c2nc3c4c(nc(OC[C@@]56CCCN5C[C@H](F)C6)nc4c2F)N([C@H](C)c2cccnc2N)CCO3)c1C(F)(F)F]

A solution of 3-((1R)-1-(5-(3-(bis(4-methoxybenzyl)amino)-2,4-difluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (120.0 mg, 0.1 mmol) in trifluoroacetic acid (1.0 mL) and trifluoromethanesulfonic acid (0.1 mL) was stirred at 25° C. for 20 minutes. After completion, the filtrate was concentrated under reduced pressure. The reaction mixture was diluted with dichloromethane, adjusted to pH=7 with saturated sodium bicarbonate solution. The resulting solution was extracted with dichloromethane, dried over anhydrous sodium sulfate and concentrated under vacuum. The residue was purified by flash chromatography on silica gel eluting with dichloromethane/methanol (10:1) to afford crude solid. The crude product was purified by Prep-HPLC with the following condition:Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 38% B to 60% B in 10 min, 60% B; Wave Length: 220/254 nm; RT1(min): 7.72 to afford the title compound (59.0 mg, 0.08 mmol, 83.3% yield).

Example 12: ¹H NMR (300 MHz, DMSO-d₆, ppm) δ 7.96 (d, J=4.9 Hz, 1H), 7.68-7.58 (m, 1H), 6.67 (dd, J=7.5, 4.9 Hz, 1H), 6.34 (d, J=7.0 Hz, 1H), 6.15 (s, 2H), 5.68 (s, 2H), 5.28 (d, J=54.5 Hz, 1H), 4.51-4.36 (m, 1H), 4.29 (dd, J=12.8, 6.3 Hz, 1H), 4.12 (s, 2H), 3.80-3.55 (m, 2H), 3.17-3.00 (m, 2H), 2.99 (s, 1H), 2.89-2.68 (m, 1H), 2.30 (s, 3H), 2.23-2.08 (m, 1H), 2.07-1.91 (m, 2H), 1.91-1.70 (m, 3H), 1.62-1.47 (m, 3H). LC-MS: (ESI, m/Z): 693.3 [M+H]⁺

### Example 13: 3-((1R)-1-(5-(3-amino-2,4-difluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine

### Synthetic Route

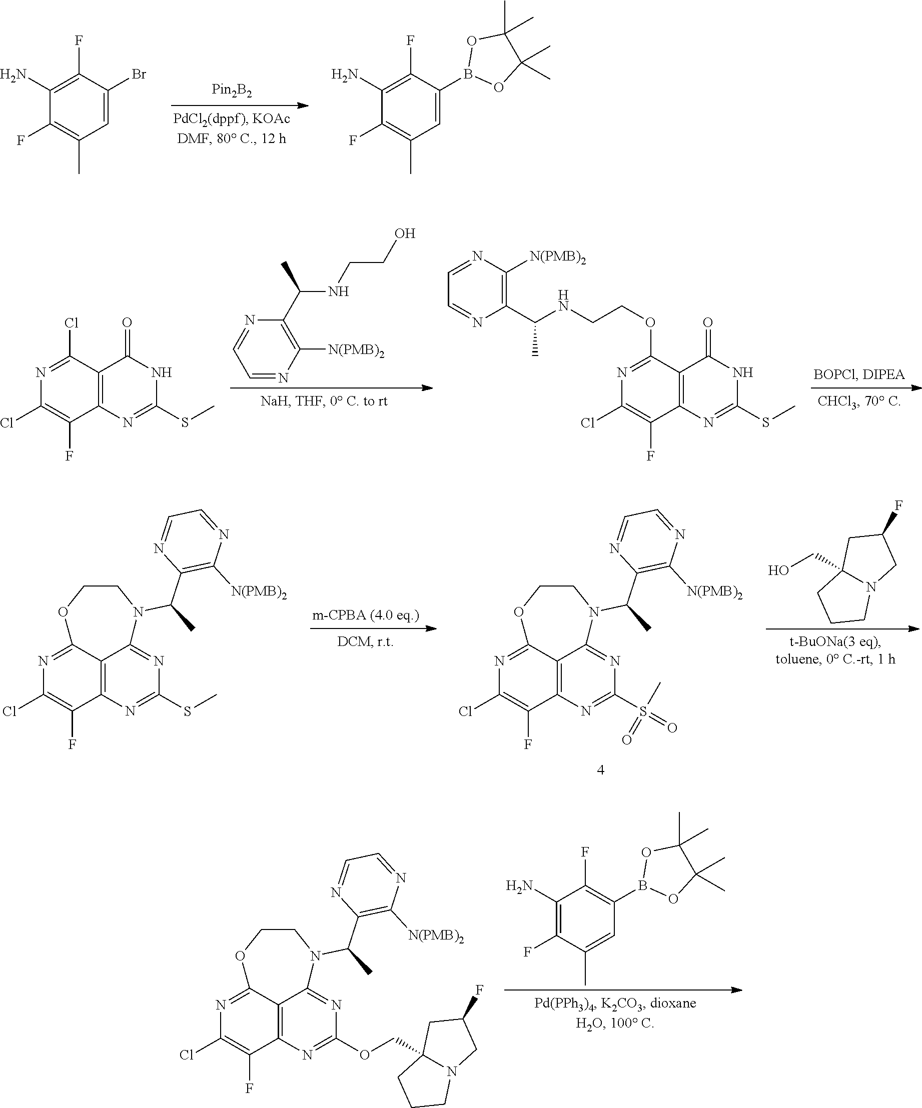

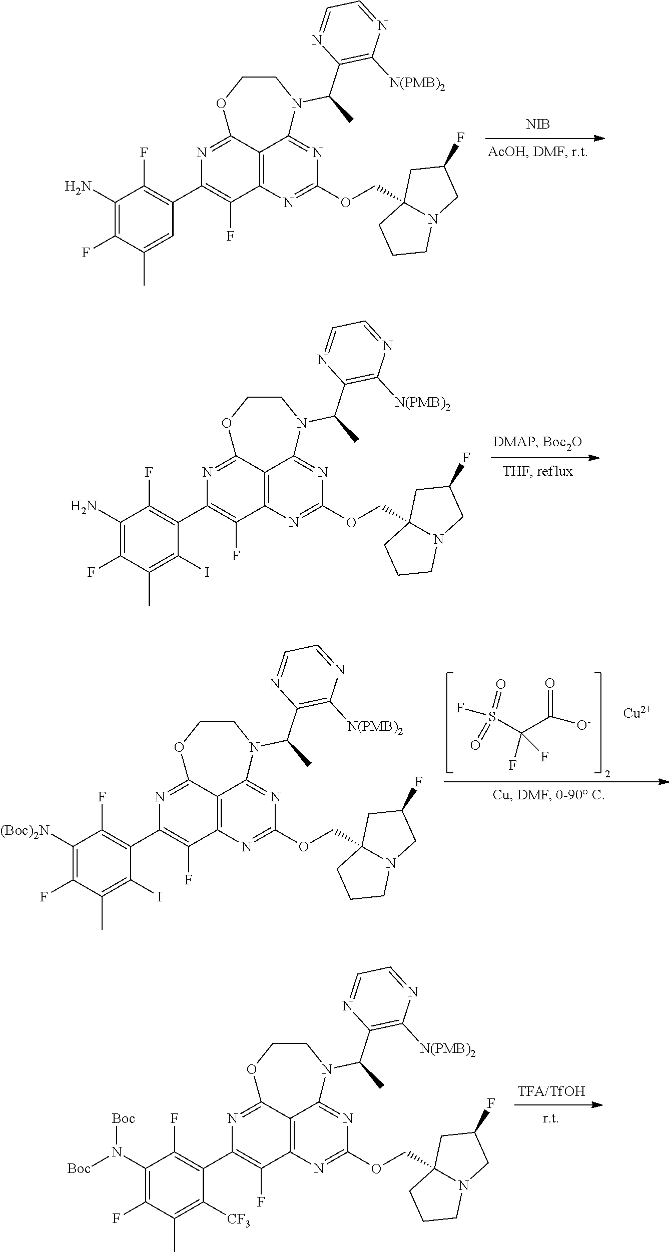

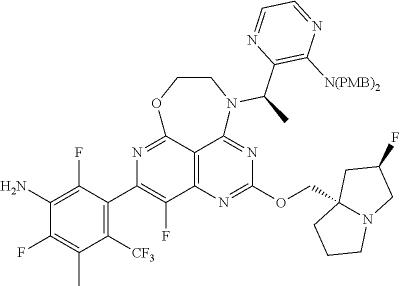

### Step 1: 2,6-difluoro-3-methyl-5-(4,4,5,5-tetramethyl-1,3,2-dioxaborolan-2-yl)aniline

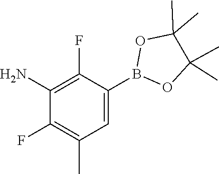 [SMILES: Cc1cc(B2OC(C)(C)C(C)(C)O2)c(F)c(N)c1F]

Under nitrogen, a solution of 3-bromo-2,6-difluoro-5-methyl-aniline (3.0 g, 13.5 mmol), bis(pinacolato)diboron (5.1 g, 20.2 mmol), [1,1′-bis(diphenylphosphino)ferrocene]dichloropalladium(II) (1.0 g, 1.3 mmol) and potassium acetate (3.9 g, 40.5 mmol) in N,N-dimethylformamide (30 mL) at 80° C. for 12 hours.

After completion, the reaction mixture was diluted with ethyl acetate, washed with water, dried over anhydrous sodium sulfate and concentrated under vacuum. The residue was purified by flash chromatography on silica gel eluting with ethyl acetate/petroleum ether (1:8) to afford 2,6-difluoro-3-methyl-5-(4,4,5,5-tetramethyl-1,3,2-dioxaborolan-2-yl)aniline (3.0 g, 11.1 mmol, 99% yield) as a yellow solid. LC-MS: (ESI, m/Z): 270.2 [M+H]⁺

### Step 2: (R)-5-(2-((1-(3-(bis(4-methoxybenzyl)amino)pyrazin-2-yl)ethyl)amino)ethoxy)-7-chloro-8-fluoro-2-(methylthio)pyrido[4,3-d]pyrimidin-4(3H)-one

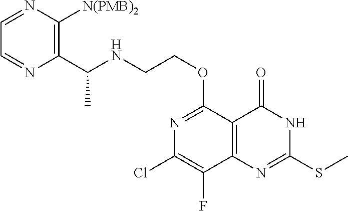

Sodium hydride (828.0 mg, 20.7 mmol, 60% in mineral oil) was added to a solution of 2-[[(1R)-1-[3-[bis[(4-methoxyphenyl)methyl]amino]pyrazin-2-yl]ethyl]amino]ethanol (3.30 g, 7.8 mmol) in tetrahydrofuran (15.0 mL) under nitrogen atmosphere at 0° C., and the mixture was stirred at 0° C. for 0.5 hour. Then the mixture was added to a solution of 5,7-dichloro-8-fluoro-2-methylsulfanyl-3H-pyrido[4,3-d]pyrimidin-4-one (1.40 g, 5.0 mmol) in tetrahydrofuran (15 mL) and stirred at room temperature for 1 hour. After completion, the reaction was quenched with sat. ammonium chloride aq. and extracted by ethyl acetate. The organic layer was washed by brine and dried over anhydrous sodium sulfate. After concentration under vacuum, the residue was used in next step without purification. LC-MS: (ESI, m/Z): 666.0 [M+H]⁺

### Step 3: (R)-3-(1-(5-chloro-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyrazin-2-amine

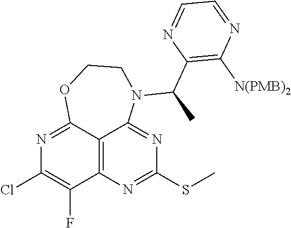

A solution of 7-chloro-8-fluoro-2-methylsulfanyl-5-[2-[[rac-(1R)-1-[3-[bis[(4-methoxyphenyl)methyl]amino]pyrazin-2-yl]ethyl]amino]ethoxy]-3H-pyrido[4,3-d]pyrimidin-4-one (2.88 g, crude), bis(2-oxo-3-oxazolidinyl)phosphinic chloride (2.2 g, 8.65 mmol) and N,N-diisopropylethylamine (3.7 mL, 21.6 mmol) in chloroform (28 mL) was stirred at 70° C. for 1 hour. After completion, the resulting solution was diluted with water and extracted with dichloromethane. The organic layers were washed with brine, dried over anhydrous sodium sulfate and concentrated. The residue was purified by flash chromatography on silica gel eluting with petroleum ether/ethyl acetate (1:1) to afford the title compound (1.58 g, 2.3 mmol, 55.1% yield) as a white solid. LC-MS: (ESI, m/Z): 648.3 [M+H]⁺

### Step 4: (R)-3-(1-(5-chloro-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyrazin-2-amine

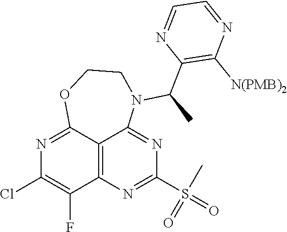

A solution of 3-[(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl]-N,N-bis[(4-methoxyphenyl)methyl]pyrazin-2-amine (300.0 mg, 0.4 mmol) and 3-chloroperoxybenzoic acid (319.5 mg, 1.8 mmol) in dichloromethane (3 mL) was stirred at 25° C. for 1 hour. After completion, the reaction was quenched with saturated sodium sulfite solution. The reaction mixture was diluted with ethyl acetate washed with water, dried over anhydrous sodium sulfate and concentrated under vacuum. The residue was purified by flash chromatography on silica gel eluting with ethyl acetate/petroleum ether (1:8) to afford the title compound (190.0 mg, 0.3 mmol, 60.4% yield) as a yellow solid. LC-MS: (ESI, m/Z): 680.3 [M+H]⁺

### Step 5: 3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine

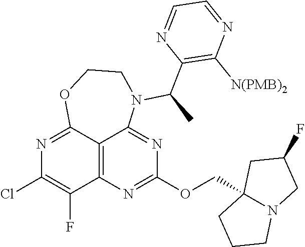

A solution of ((2R,7aS)-2-fluorotetrahydro-1H-pyrrolizin-7a(5H)-yl)methanol (1.33 g, 8.3 mmol) and sodium tert-butoxide(1.34 g, 13.8 mmol) in toluene (20 mL) was stirred at 0° C. for 20 minutes. Then the reaction mixture was added into a solution of (R)-3-(1-(5-chloro-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyrazin-2-amine (1.89 g, 2.7 mmol) in toluene (20 mL) and stirred at 25° C. for 1.5 hours. After completion, the reaction was quenched with saturated ammonium chloride solution, diluted with ethyl acetate, washed with water, dried over anhydrous sodium sulfate and concentrated under vacuum. The residue was purified by flash chromatography on silica gel eluting with ethyl acetate/petroleum ether (1:8) to afford the title compound (910.0 mg, 1.1 mmol, 43.1% yield) as a yellow solid. LC-MS: (ESI, m/Z): 759.3 [M+H]⁺

### Step 6: 3-((R)-1-(5-(3-amino-2,4-difluoro-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine

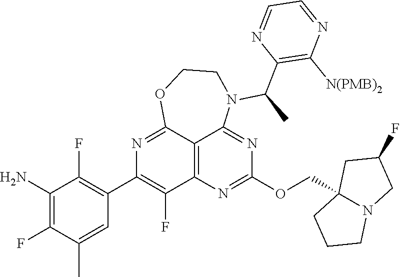

Under nitrogen, a solution of 3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine (910.0 mg, 1.2 mmol), 2,6-difluoro-3-methyl-5-(4,4,5,5-tetramethyl-1,3,2-dioxaborolan-2-yl)aniline (387.0 mg, 1.4 mmol), tetrakis(triphenylphosphine)palladium (138.5 mg, 0.12 mmol) and potassium carbonate (331.2 mg, 2.4 mmol) in 1,4-dioxane(10 mL) and water (1 mL) was stirred at 100° C. for 1.5 hours. After completion, the reaction mixture was diluted with water, extracted with ethyl acetate, dried over anhydrous sodium sulfate and concentrated under vacuum. The residue was purified by flash chromatography on silica gel eluting with dichloromethane/methanol (10:1) to afford the title compound (330.0 mg, 0.4 mmol, 31.8% yield) as a yellow oil. LC-MS: (ESI, m/Z): 866.3 [M+H]⁺

### Step 7: 3-((1R)-1-(5-(3-amino-2,4-difluoro-6-iodo-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine

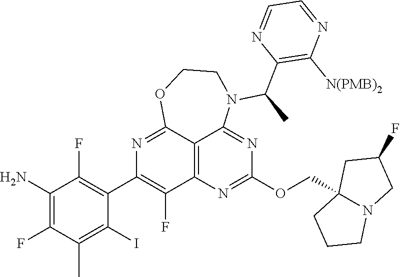

A solution of 3-((R)-1-(5-(3-amino-2,4-difluoro-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine (600.0 mg, 0.7 mmol) and N-iodosuccinimide (155.4 mg, 0.7 mmol) in acetic acid (3.0 mL) and N,N-dimethylformamide (3.0 mL) was stirred at 25° C. for 1.5 hours. After completion, the reaction was quenched with saturatedsodium carbonate solution. The reaction mixture was diluted with water, extracted with ethyl acetate and the organic layers were combined. The organic layer was dried over anhydrous sodium sulfate and concentrated under vacuum. The residue was purified by flash chromatography on silica gel eluting with ethyl acetate/petroleum ether (1:8) to afford the title compound (500.0 mg, 0.5 mmol, 72.8% yield) as a yellow solid. LC-MS: (ESI, m/Z): 992.4 [M+H]⁺

### Step 8: tert-butyl N-[3-[13-[(1R)-1-[3-[bis[(4-methoxyphenyl)methyl]amino]pyrazin-2-yl]ethyl]-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-7-yl]-2,6-difluoro-4-iodo-5-methyl-phenyl]-N-tert-butoxycarbonyl-carbamate

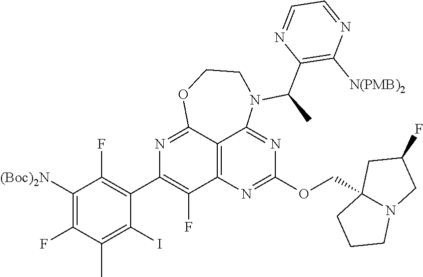

Under nitrogen, a solution of 3-((1R)-1-(5-(3-amino-2,4-difluoro-6-iodo-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine (500.0 mg, 0.5 mmol), 4-dimethylaminopyridine (215.6 mg, 1.7 mmol) and di-tert-butyl dicarbonate (1.1 g, 5.0 mmol) in tetrahydrofuran was stirred at 70° C. for 1 hour. After completion, the reaction mixture was diluted with water, extracted with ethyl acetate and washed with water. The organic layer was dried over anhydrous sodium sulfate and concentrated under vacuum. The residue was purified by flash chromatography on silica gel eluting with methanol/dichloromethane (1:8) to afford the title compound (430.0 mg, 0.3 mmol, 71.6% yield) as a white solid. LC-MS: (ESI, m/Z): 866.3 [M+H]⁺

### Step 9: tert-butyl N-[3-[13-[(1R)-1-[3-[bis[(4-methoxyphenyl)methyl]amino]pyrazin-2-yl]ethyl]-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-7-yl]-2,6-difluoro-5-methyl-4-(trifluoromethyl)phenyl]-N-tert-butoxycarbonyl-carbamate

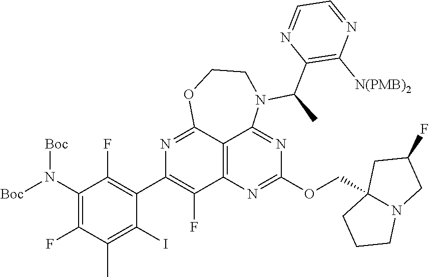

Under nitrogen, a solution of tert-butyl N-[3-[13-[(1R)-1-[3-[bis[(4-methoxyphenyl)methyl]amino]pyrazin-2-yl]ethyl]-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-7-yl]-2,6-difluoro-4-iodo-5-methyl-phenyl]-N-tert-butoxycarbonyl-carbamate (430.0 mg, 0.3 mmol) and copper (230.8 mg, 3.6 mmol) in N,N-dimethylformamide (4.5 mL) was added bis[(2,2-difluoro-2-fluorosulfonyl-acetyl)oxy]copper (868.0 mg, 3.6 mmol) at −60° C. for 8 minutes. Then the reaction mixture was stirred at 90° C. for 1.5 hours. After completion, the reaction mixture was diluted with water, extracted with ethyl acetate, washed with water, dried over anhydrous sodium sulfate and concentrated under vacuum. The residue was purified by flash chromatography on silica gel eluting with methanol/dichloromethane (1:8) to afford the title compound (160.0 mg, 0.14 mmol, 39.1% yield) as a white solid. LC-MS: (ESI, m/Z): 1134.6 [M+H]⁺

### Step 10: 3-((1R)-1-(5-(3-amino-2,4-difluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine

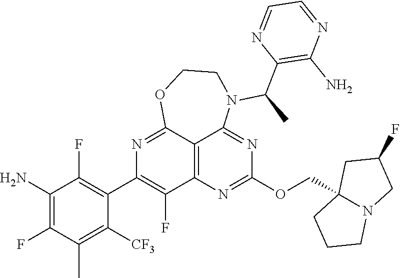 [SMILES: Cc1c(F)c(N)c(F)c(-c2nc3c4c(nc(OC[C@@]56CCCN5C[C@H](F)C6)nc4c2F)N([C@H](C)c2nccnc2N)CCO3)c1C(F)(F)F]

A solution of tert-butyl N-[3-[13-[(1R)-1-[3-[bis[(4-methoxyphenyl)methyl]amino]pyrazin-2-yl]ethyl]-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-7-yl]-2,6-difluoro-5-methyl-4-(trifluoromethyl)phenyl]-N-tert-butoxycarbonyl-carbamate (160.0 mg, 0.1 mmol) in trifluoroacetic acid (1.0 mL) and trifluoromethanesulfonic acid (0.1 mL) was stirred at 25° C. for 30 minutes. After completion, the reaction mixture was diluted with dichloromethane, adjusted to pH=7 with saturated sodium bicarbonate solution. The resulting solution was extracted with dichloromethane, dried over anhydrous sodium sulfate and concentrated under vacuum. The residue was purified by flash chromatography on silica gel eluting with dichloromethane/methanol (10:1) to afford the product. The product was further purified by Prep-HPLC with the following conditions (Column: XSelect CSH Fluoro Phenyl, 30*150 mm, 5 μm; Mobile Phase A: Water(0.1% FA), Mobile Phase B: ACN; Flow rate: 60 mL/min; Gradient: 4% B to 20% B in 10 min; Wave Length: 694 nm) to afford the title compound (11.0 mg, 0.01 mmol, 10.5% yield). LC-MS: (ESI, m/Z): 694.0 [M+H]⁺

Example 13: ¹H NMR (300 MHz, DMSO-d₆, ppm) δ 7.94 (t, J=2.3 Hz, 1H), 7.78 (t, J=2.6 Hz, 1H), 6.37 (s, 3H), 6.17 (s, 2H), 5.28 (d, J=54.4 Hz, 1H), 4.65-4.34 (m, 2H), 4.22-4.02 (m, 2H), 3.99-3.83 (m, 1H), 3.80-3.61 (m, 1H), 3.17-2.97 (m, 2H), 2.98-2.88 (m, 1H), 2.86-2.75 (m, 1H), 2.31 (s, 3H), 2.19-1.93 (m, 3H), 1.89-1.68 (m, 3H), 1.56 (dd, J=6.9, 2.1Hz, 3H). LCMS (ESI, m/Z): 694.0 [M+H]⁺.

### Example 14: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-6-methyl-5-(trifluoromethyl)pyridin-2-amine

### Synthetic Route

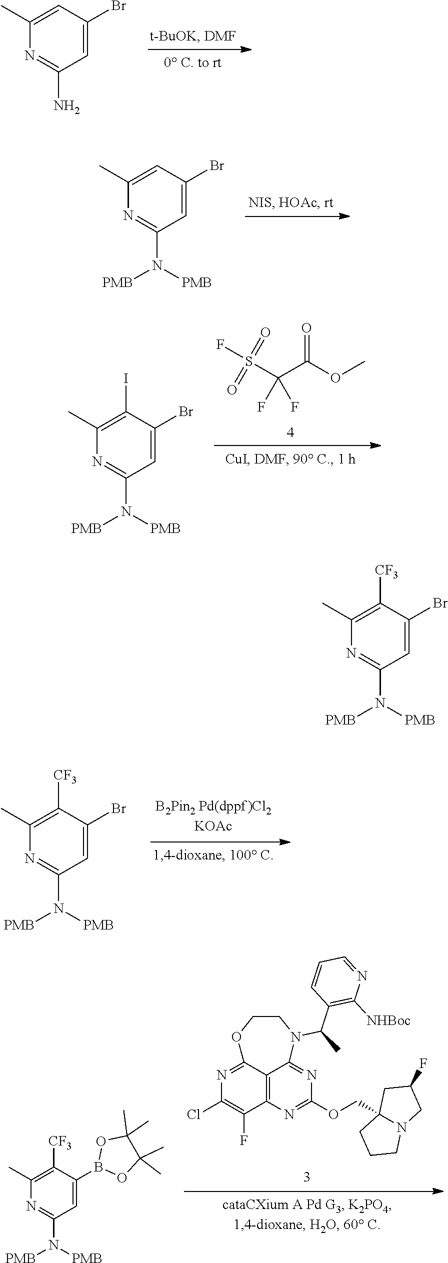

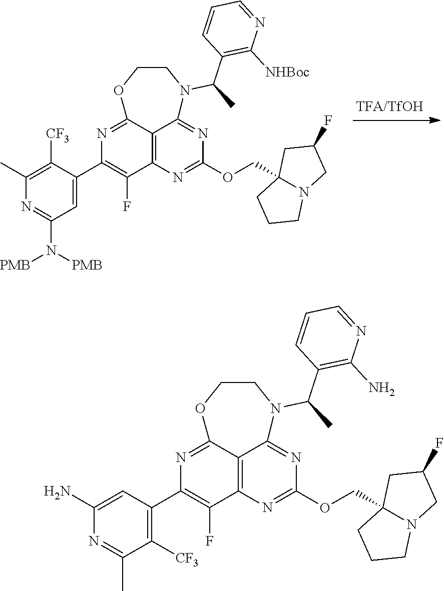

### Step 1: 4-bromo-N,N-bis(4-methoxybenzyl)-6-methylpyridin-2-amine

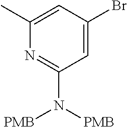

A solution of 4-bromo-6-methyl-pyridin-2-amine (2.0 g, 10.6 mmol) and potassium tert-butoxide (4.80 g, 42.7 mmol) in N,N-dimethylformamide (20 mL) was stirred at 0° C. for 30 minutes. Then 4-methoxybenzylchloride (5.02 g, 32.0 mmol) in N,N-dimethylformamide (20 mL) was added and stirred at 0° C. for 1.5 hours. After completion, the reaction was quenched with saturated ammonium chloride solution. The reaction mixture was diluted with water, extracted with ethyl acetate, dried over anhydrous sodium sulfate and concentrated under vacuum. The residue was purified by flash chromatography on silica gel eluting with ethyl acetate/petroleum ether (1:99) to afford the title compound (1.50 g, 3.5 mmol, 32.8% yield) as a white solid. LCMS (ESI, m/Z): 429.1 [M+H]⁺.

### Step 2: 4-bromo-5-iodo-N,N-bis(4-methoxybenzyl)-6-methylpyridin-2-amine

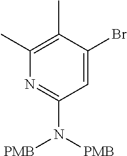

A solution of 4-bromo-N,N-bis[(4-methoxyphenyl)methyl]-6-methyl-pyridin-2-amine (2.09 g, 4.8 mmol) and N-iodosuccinimide (1.10 g, 4.8 mmol) in acetic acid (20 mL) was stirred at 25° C. for 1.5 hours. After completion, the reaction was quenched with sat. sodium carbonate aq. The reaction mixture was diluted with ethyl acetate, washed with water, dried over anhydrous sodium sulfate and concentrated under vacuum. The residue was purified by flash chromatography on silica gel eluting with ethyl acetate/petroleum ether (1:98) to afford the title compound (1.50 g, 2.7 mmol, 55.4% yield) as a yellow oil. LCMS (ESI, m/Z): 554.1 [M+H]⁺.

### Step 3: 4-bromo-N,N-bis(4-methoxybenzyl)-6-methyl-5-(trifluoromethyl)pyridin-2-amine

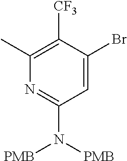

Under nitrogen, a solution of 4-bromo-5-iodo-N,N-bis[(4-methoxyphenyl)methyl]-6-methyl-pyridin-2-amine (1.50 g, 2.7 mmol), methyl 2,2-difluoro-2-(fluorosulfonyl)acetate (3.4 mL, 27.1 mmol) and cuprous iodide (5.16 g, 27.1 mmol) in N,N-dimethylformamide (15 mL) was stirred at 90° C. for 1.5 hours. After completion, the reaction mixture was filtered and the filter was diluted with water. The resulting solution was extracted with ethyl acetate, washed with water, dried over anhydrous sodium sulfate and concentrated under vacuum. The residue was purified by flash chromatography on silica gel eluting with ethyl acetate/petroleum ether (1:19) to afford the title compound (1.14 g, 2.3 mmol, 84.9% yield) as a yellow solid. LCMS (ESI, m/Z): 497.2 [M+H]⁺.

### Step 4: N,N-bis(4-methoxybenzyl)-6-methyl-4-(4,4,5,5-tetramethyl-1,3,2-dioxaborolan-2-yl)-5-(trifluoromethyl)pyridin-2-amine

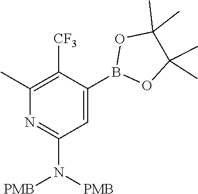

Under nitrogen, a solution of 4-bromo-N,N-bis[(4-methoxyphenyl)methyl]-6-methyl-5-(trifluoromethyl)pyridin-2-amine (150.0 mg, 0.3 mmol),bis(pinacolato)diboron (115.3 mg, 0.4 mmol),[1,1′-Bis(diphenylphosphino)ferrocene]dichloropalladium(II) (22.4 mg, 0.03 mmol) and potassium acetate (0.04 mL, 0.6 mmol) in 1,4-dioxane (1.5 mL) was added at 100° C. for 1.5 hours. After completion, the reaction mixture was used in the next step directly without further purification. LCMS (ESI, m/Z):543.4 [M+H]⁺.

### Step 5: tert-butyl (3-((R)-1-(5-(6-(bis(4-methoxybenzyl)amino)-2-methyl-3-(trifluoromethyl)pyridin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate

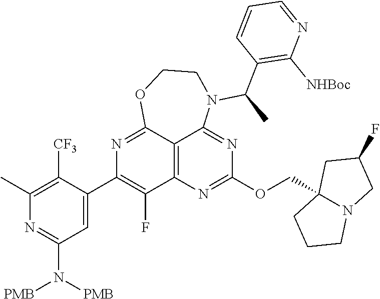

Under nitrogen, a solution of N,N-bis[(4-methoxyphenyl)methyl]-6-methyl-4-(4,4,5,5-tetramethyl-1,3,2-dioxaborolan-2-yl)-5-(trifluoromethyl)pyridin-2-amine (150.0 mg, crude), tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (170.9 mg, 0.3 mmol), mesylate[(di(1-adamantyl)-n-butylphosphine)-2-(2′-amino-1,1′-biphenyl)]palladium(II) (40.2 mg, 0.06 mmol) and potassium phosphate (117.26 mg, 0.55 mmol) in 1,4-dioxane (2.5 mL) and water (0.5 mL) was stirred at 60° C. for 2 hours.

After completion, the reaction mixture was diluted with water. The resulting solution was extracted with ethyl acetate, washed with water. The organic layer was dried over anhydrous sodium sulfate and concentrated under vacuum. The residue was purified by flash chromatography on silica gel eluting with methanol/dichloromethane (1:8) to afford the title compound (43.0 mg, 0.04 mmol, 15.6% yield). LCMS (ESI, m/Z): 998.5 [M+H]⁺.

### Step 5: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-6-methyl-5-(trifluoromethyl)pyridin-2-amine

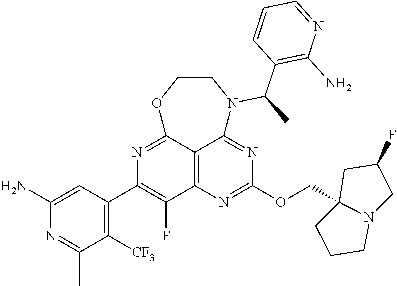 [SMILES: Cc1nc(N)cc(-c2nc3c4c(nc(OC[C@@]56CCCN5C[C@H](F)C6)nc4c2F)N([C@H](C)c2cccnc2N)CCO3)c1C(F)(F)F]

A solution of tert-butyl (3-((R)-1-(5-(6-(bis(4-methoxybenzyl)amino)-2-methyl-3-(trifluoromethyl)pyridin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (80.0 mg, 0.08 mmol) in trifluoroacetic acid (0.5 mL) and trifluoromethanesulfonic acid (0.05 mL) was stirred at 25° C. for 30 minutes. After completion, the filtrate was concentrated under reduced pressure. The reaction mixture was diluted with dichloromethane and the reaction mixture was adjusted to pH=7 with saturated sodium bicarbonate solution. The resulting solution was extracted with dichloromethane, dried over anhydrous sodium sulfate and concentrated under vacuum.

The residue was purified by flash chromatography on silica gel eluting with dichloromethane/methanol (10:1) to afford crude solid. The crude product was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 50% B in 7.5 min; Wave Length: 220 nm) to afford the title compound (18.0 mg, 0.03 mmol, 34.1% yield) as a white solid.

Example 14: ¹H NMR (300 MHz, DMSO-d₆, ppm) δ 7.96 (dd, J=4.9, 1.7 Hz, 1H), 7.62 (d, J=7.4 Hz, 1H), 6.83 (s, 2H), 6.66 (dd, J=7.5, 4.9 Hz, 1H), 6.33 (q, J=6.7 Hz, 1H), 6.20 (s, 1H), 5.69 (s, 2H), 5.28 (d, J=54.4 Hz, 1H), 4.42 (dd, J=12.9, 6.4 Hz, 1H), 4.28 (dd, J=12.6, 6.3 Hz, 1H), 4.12 (s, 2H), 3.82-3.60 (m, 2H), 3.17-3.02 (m, 2H), 2.99 (s, 1H), 2.90-2.70 (m, 1H), 2.46 (d, J=2.3 Hz, 3H), 2.19-2.11 (m, 1H), 2.09-1.95 (m, 2H), 1.92-1.71 (m, 3H), 1.55 (d, J=6.8 Hz, 3H). LCMS (ESI, m/Z): 658.3[M+H]⁺.

### Example 15: 3-((1R)-1-(5-(3-amino-2-fluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Synthetic Route

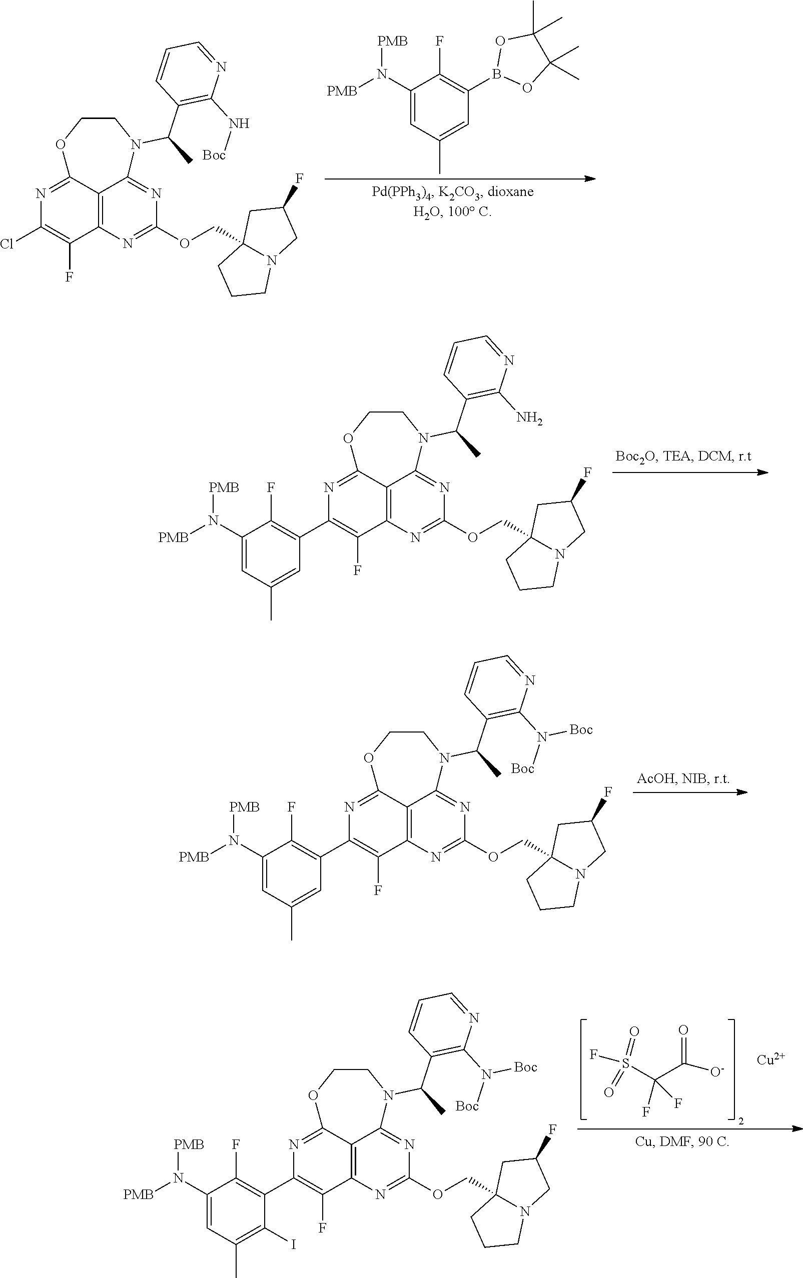

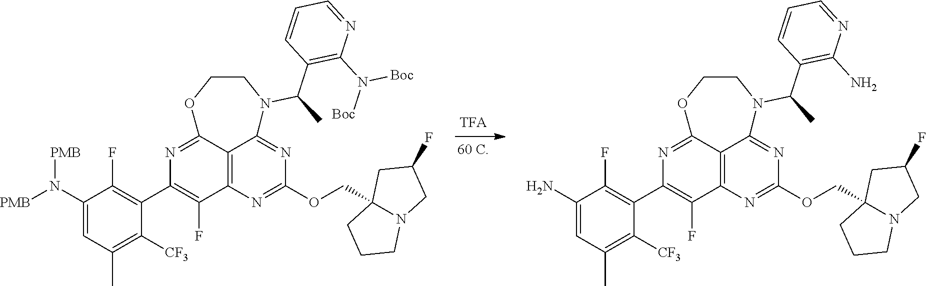

### Step 1: 3 ((R) 1 (5-(3-(bis(4 methoxybenzyl)amino)-2 fluoro 5 methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

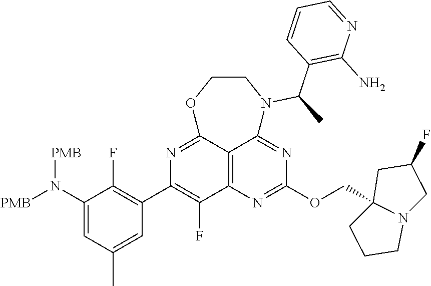

Under nitrogen, a solution of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (420.0 mg, 0.58 mmol), 2-fluoro-N,N-bis(4-methoxybenzyl)-5-methyl-3-(4,4,5,5-tetramethyl-1,3,2-dioxaborolan-2-yl)aniline (431.0 mg, 0.88 mmol), potassium carbonate (242.4 mg, 1.75 mmol) and tetrakis(triphenylphosphine)palladium (67.5 mg, 0.06 mmol) in 1,4-dioxane (3 mL) and water (0.60 mL) was stirred at 100° C. for 2 hours. After completion, the reaction solution was diluted with water and extracted with ethyl acetate. The organic layers were washed with brine, dried over anhydrous sodium sulfate and concentrated.

The residue was purified by flash chromatography on silica gel eluting with petroleum ether/ethyl acetate (1:1) to afford 3-((R)-1-(5-(3-(bis(4-methoxybenzyl)amino)-2-fluoro-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (400.0 mg, 0.47 mmol, 80.8% yield) as a yellow solid. LC-MS: (ESI, m/Z): 847.4 [M+H]⁺.

### Step 2: tert-butyl N-tert-butoxycarbonyl-N-[3-[rac-(1R)-1-[7-[3-[bis[(4-methoxyphenyl)methyl]amino]-2-fluoro-5-methyl-phenyl]-6-fluoro-3-[[rac-(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]carbamate

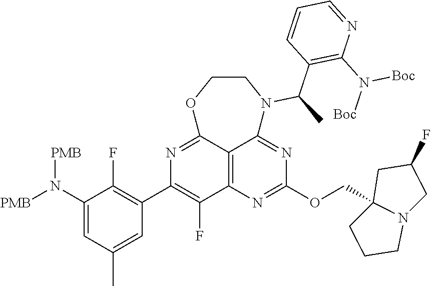

A solution of 3-((R)-1-(5-(3-(bis(4-methoxybenzyl)amino)-2-fluoro-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (400.0 mg, 0.47 mmol), di-tert-butyl dicarbonate (515.3 mg, 2.36 mmol) and triethylamine (0.33 mL, 2.36 mmol) in dichloromethane (4 mL) was stirred at 25° C. for 1 hour. After completion, the reaction solution was diluted with water and extracted with dichloromethane. The organic layers were washed with brine, dried over anhydrous sodium sulfate and concentrated. The residue was purified by flash chromatography on silica gel eluting with petroleum ether/ethyl acetate (1:1) to afford tert-butyl N-tert-butoxycarbonyl-N-[3-[rac-(1R)-1-[7-[3-[bis[(4-methoxyphenyl)methyl]amino]-2-fluoro-5-methyl-phenyl]-6-fluoro-3-[[rac-(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]carbamate (250.0 mg, 0.22 mmol, 47.3% yield) as a yellow solid. LC-MS: (ESI, m/Z): 1047.5 [M+H]⁺.

### Step 3: tert-butyl N-tert-butoxycarbonyl-N-[3-[rac-(1R)-1-[7-[3-[bis[(4-methoxyphenyl)methyl]amino]-2-fluoro-6-iodo-5-methyl-phenyl]-6-fluoro-3-[[rac-(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]carbamate

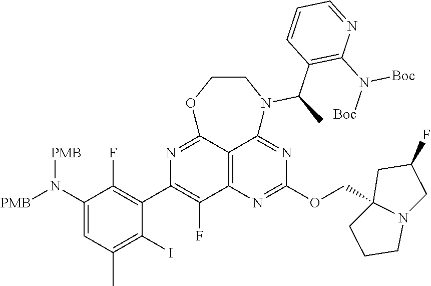

To a solution of tert-butyl N-tert-butoxycarbonyl-N-[3-[rac-(1R)-1-[7-[3-[bis[(4-methoxyphenyl)methyl]amino]-2-fluoro-5-methyl-phenyl]-6-fluoro-3-[[rac-(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]carbamate (240.0 mg, 0.23 mmol) in acetic acid (2.5 mL) was added N-iodosuccinimide (61.8 mg, 0.28 mmol), the mixture was stirred at 25° C. for 0.5 hour. After completion, the reaction was quenched with saturated sodium thiosulfate solution. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1/1) to afford tert-butyl N-tert-butoxycarbonyl-N-[3-[rac-(1R)-1-[7-[3-[bis[(4-methoxyphenyl)methyl]amino]-2-fluoro-6-iodo-5-methyl-phenyl]-6-fluoro-3-[[rac-(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]carbamate (140.0 mg, 0.10 mmol, 47.9% yield) as a yellow solid. LC-MS: (ESI, m/Z): 1173.4 [M+H]⁺.

### Step 4:tert-butyl N-tert-butoxycarbonyl-N-[3-[rac-(1R)-1-[7-[3-[bis[(4-methoxyphenyl)methyl]amino]-2-fluoro-5-methyl-6-(trifluoromethyl)phenyl]-6-fluoro-3-[[rac-(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]carbamate

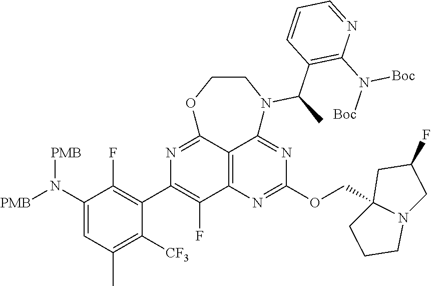

Under nitrogen, to a solution of tert-butyl N-tert-butoxycarbonyl-N-[3-[rac-(1R)-1-[7-[3-[bis[(4-methoxyphenyl)methyl]amino]-2-fluoro-6-iodo-5-methyl-phenyl]-6-fluoro-3-[[rac-(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]carbamate (140.0 mg, 0.12 mmol) and copper (75.8 mg, 1.19 mmol) in N,N-dimethylformamide (1 mL) was added bis[(2,2-difluoro-2-fluorosulfonyl-acetyl)oxy]copper (498.8 mg, 1.19 mmol) and stirred at −78° C. for 10 minutes, then the mixture was removed to 90° C. and stirred for 1 hour. After completion, the resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1/1) to afford tert-butyl N-tert-butoxycarbonyl-N-[3-[rac-(1R)-1-[7-[3-[bis[(4-methoxyphenyl)methyl]amino]-2-fluoro-5-methyl-6-(trifluoromethyl)phenyl]-6-fluoro-3-[[rac-(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]carbamate (110.0 mg, 0.097 mmol, 81.8% yield) as a yellow solid. LC-MS: (ESI, m/Z): 1115.5 [M+H]⁺.

### Step 5: 3-((1R)-1-(5-(3-amino-2-fluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

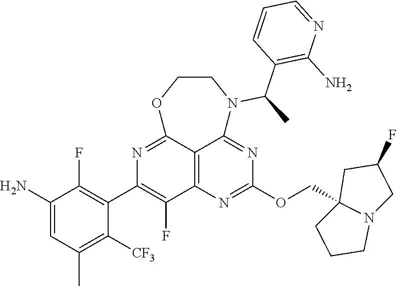 [SMILES: Cc1cc(N)c(F)c(-c2nc3c4c(nc(OC[C@@]56CCCN5C[C@H](F)C6)nc4c2F)N([C@H](C)c2cccnc2N)CCO3)c1C(F)(F)F]

A solution of tert-butyl N-tert-butoxycarbonyl-N-[3-[rac-(1R)-1-[7-[3-[bis[(4-methoxyphenyl)methyl]amino]-2-fluoro-5-methyl-6-(trifluoromethyl)phenyl]-6-fluoro-3-[[rac-(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]carbamate (110.0 mg, 0.10 mmol) in 2,2,2-trifluoroacetic acid (1 mL) was stirred at 50° C. for 5 hours. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The resulting residue was purified by reverse phase chromatography (acetonitrile/0.1% ammonium bicarbonate in water) to afford crude product. The product was further purified by Prep-HPLC with the following conditions: XSelect CSH Fluoro Phenyl, 30*150 mm, 5 μm; Mobile Phase A: water (10 mmol/L NH₄HCO₃), Mobile Phase B: acetonitrile; Flow rate: 60 mL/min; Gradient: 31% B to 56% B in 9 min; 254/220 nm; RT1:10.4 min to afford 3-((1R)-1-(5-(3-amino-2-fluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (6.5 mg, 0.01 mmol, 9.2% yield). LC-MS: (ESI, m/Z): 675.2 [M+H]⁺.

Example 15: ¹H NMR (300 MHz, Methanol-d₄) δ8.14-7.89 (m, 1H), 7.95-7.65 (m, 1H), 6.87 (d, J=8.8 Hz, 1H), 6.80 (dd, J=7.5, 5.1Hz, 1H), 6.62 (q, J=6.9 Hz, 1H), 5.33 (d, J=53.2 Hz, 1H), 4.55-4.42 (m, 1H), 4.42-4.27 (m, 3H), 3.78 (dd, J=16.1, 6.6 Hz, 1H), 3.63-3.48 (m, 1H), 3.33-3.13 (m, 3H), 3.11-2.94 (m, 1H), 2.42 (d, J=2.7 Hz, 3H), 2.37-2.12 (m, 3H), 2.13-1.82 (m, 3H), 1.67 (d, J=7.0 Hz, 3H).

### Example 16: 3-((R)-1-(5-(5-amino-4-fluoro-3-methyl-2-(trifluoromethyl)phenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine

### Synthetic Route

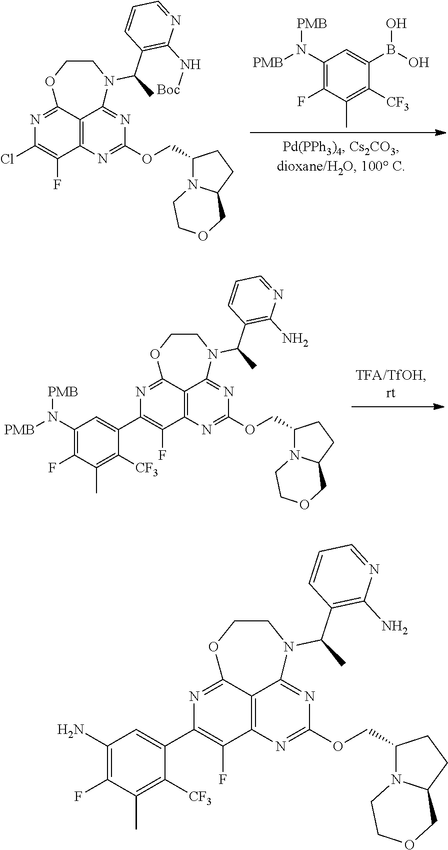

### Step 1: 3-((R)-1-(5-(5-(bis(4-methoxybenzyl)amino)-4-fluoro-3-methyl-2-(trifluoromethyl)phenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine

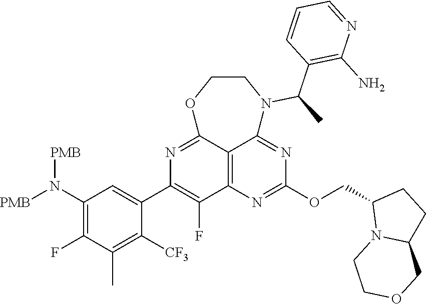

Under nitrogen, a solution of tetrakis(triphenylphosphine)palladium (86.8 mg, 0.08 mmol), cesium carbonate (367.0 mg, 1.13 mmol), [5-[bis[(4-methoxyphenyl)methyl]amino]-4-fluoro-3-methyl-2-(trifluoromethyl)phenyl]boronic acid (319.2 mg, 0.67 mmol) and tert-butyl (3-((R)-1-(5-chloro-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-yl)carbamate (240.0 mg, 0.34 mmol) in 1,4-dioxane (2 mL) and water (0.4 mL) was stirred at 100° C. for 2 hours. After completion, the reaction was diluted with water and extracted with ethyl acetate. The organic layers were combined, washed with brine, dried over anhydrous sodium sulfate and concentrated. under vacuum. The residue was purified by flash chromatography on silica gel eluting with petroleum ether/ethyl acetate (1:1) to afford 3-((R)-1-(5-(5-(bis(4-methoxybenzyl)amino)-4-fluoro-3-methyl-2-(trifluoromethyl)phenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine (240.0 mg, 0.20 mmol, 60.8% yield) as a yellow solid. LC-MS: (ESI, m/Z): 913.4 [M+H]⁺.

### Step 2: 3-((R)-1-(5-(5-amino-4-fluoro-3-methyl-2-(trifluoromethyl)phenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine

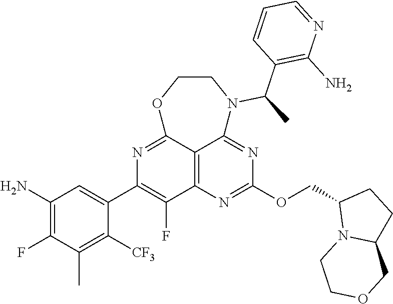 [SMILES: Cc1c(F)c(N)cc(-c2nc3c4c(nc(OC[C@@H]5CC[C@H]6COCCN65)nc4c2F)N([C@H](C)c2cccnc2N)CCO3)c1C(F)(F)F]

A solution of 3-((R)-1-(5-(5-(bis(4-methoxybenzyl)amino)-4-fluoro-3-methyl-2-(trifluoromethyl)phenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine (230.0 mg, 0.25 mmol) in trifluoromethanesulfonic acid (0.3 mL) and 2,2,2-trifluoroacetic acid (3 mL) was stirred at 25° C. for 1 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated to afford crude product. The crude product was purified by Prep-HPLC with the following conditions: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 10% B to 40% B in 9 min; 254/220 nm; RT1:8.9 min to afford 3-((R)-1-(5-(5-amino-4-fluoro-3-methyl-2-(trifluoromethyl)phenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine (81.2 mg, 0.11 mmol, 44.0% yield). LC-MS: (ESI, m/Z): 673.1 [M+H]⁺.

Example 16: ¹H NMR (400 MHz, DMSO-d₆) δ7.97 (dd, J=4.9, 1.7 Hz, 1H), 7.62 (dd, J=7.5, 1.8 Hz, 1H), 6.67 (dd, J=7.5, 4.9 Hz, 1H), 6.54 (d, J=8.7 Hz, 1H), 6.33 (q, J=6.8 Hz, 1H), 6.02 (s, 2H), 5.68 (s, 2H), 4.42-4.38 (m, 2H), 4.26-4.22 (m, 2H), 3.72 (dd, J=15.9, 6.5 Hz, 1H), 3.67-3.36 (m, 5H), 3.14 (t, J=10.4 Hz, 1H), 3.05-2.78 (m, 3H), 2.32 (s, 3H), 2.22-1.97 (m, 1H), 1.86-1.70 (m, 1H), 1.70-1.48 (m, 4H), 1.43-1.20 (m, 1H).

### Example 17: 4-(10-((R)-1-(2-aminopyridin-3-yl)ethyl)-4-fluoro-2-(((6S,8aS)-hexahydro-1H-pyrrolo[2,1-c][1,4]oxazin-6-yl)methoxy)-9,10-dihydro-8H-7-oxa-1,3,6,10-tetraazacyclohepta[de]naphthalen-5-yl)-5-ethynyinaphthalen-2-ol

### Synthetic Route

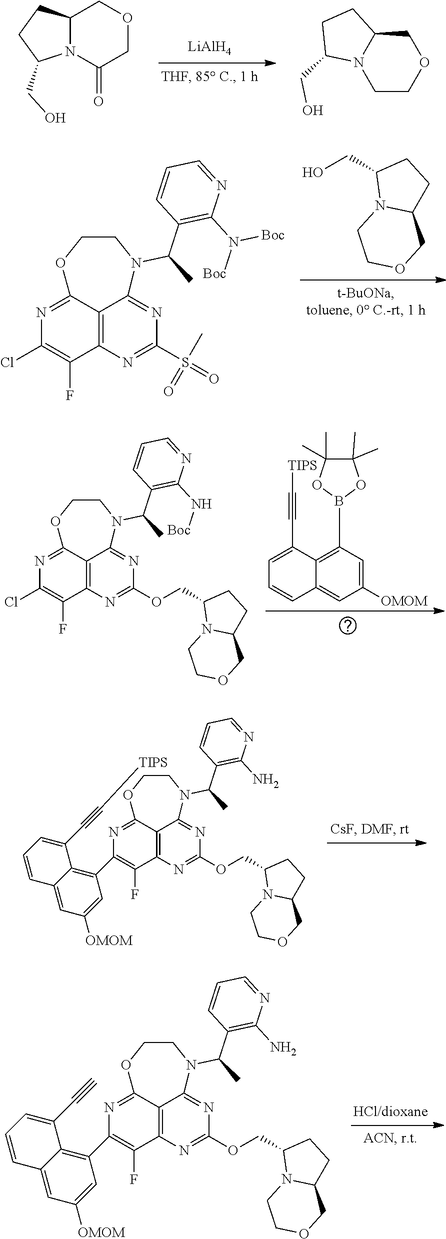

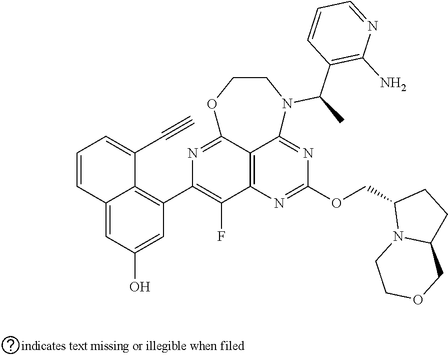 [SMILES: C#Cc1cccc2cc(O)cc(-c3nc4c5c(nc(OC[C@@H]6CC[C@H]7COCCN76)nc5c3F)N([C@H](C)c3cccnc3N)CCO4)c12]

### Step 1: ((6S,8aS)-hexahydro-1H-pyrrolo[2,1-c][1,4]oxazin-6-yl)methanol

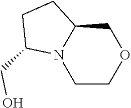 [SMILES: OC[C@@H]1CC[C@H]2COCCN12]

To a solution of (6S,8aS)-6-(hydroxymethyl)tetrahydro-1H-pyrrolo[2,1-c][1,4]oxazin-4(3H)-one (1.6 g, 9.35 mmol) in tetrahydrofuran (16 mL) was added lithium aluminum hydride (2.1 g, 56.07 mmol) at 0° C., the mixture was stirred at 65° C. for 1 hour. The reaction was quenched with sodium sulfate decahydrate. The solids were filtered out. After filtration, the filtrate was concentrated under reduced pressure to afford ((6S,8aS)-hexahydro-1H-pyrrolo[2,1-c][1,4]oxazin-6-yl)methanol (650 mg, 4.13 mmol, 44.2% yield) as a pink oil. LC-MS: (ESI, m/Z): 158.1 [M+H]⁺

### Step 2: tert-butyl (3-((R)-1-(5-chloro-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-yl)carbamate

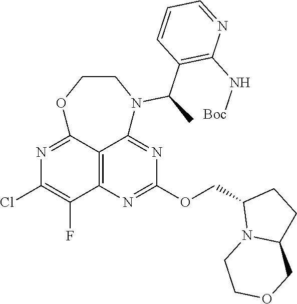 [SMILES: C[C@H](c1cccnc1NC(=O)OC(C)(C)C)N1CCOc2nc(Cl)c(F)c3nc(OC[C@@H]4CC[C@H]5COCCN54)nc1c23]

Under nitrogen, to a solution of ((6S,8aS)-hexahydro-1H-pyrrolo[2,1-c][1,4]oxazin-6-yl)methanol (812.0 mg, 5.17 mmol) in toluene (11 mL) was added sodium tert-butoxide (497.0 mg, 5.17 mmol), the mixture was stirred at 0° C. for 10 minutes. Thentert-butyl N-tert-butoxycarbonyl-N-[3-[(1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl]-2-pyridyl]carbamate (1.1 g, 1.72 mmol) was added and stirred at room temperature for 1 hour. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (24:1) to afford tert-butyl (3-((R)-1-(5-chloro-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-yl)carbamate (690.0 mg, 1.12 mmol, 65.1% yield) as a yellow solid. LC-MS: (ESI, m/Z): 616.2 [M+H]⁺

### Step 3: 3-((R)-1-(4-fluoro-2-(((6S,8aS)-hexahydro-1H-pyrrolo[2,1-c][1,4]oxazin-6-yl)methoxy)-5-(3-(methoxymethoxy)-8-((triisopropylsilyl)ethynyl)naphthalen-1-yl)-8,9-dihydro-10H-7-oxa-1,3,6,10-tetraazacyclohepta[de]naphthalen-10-yl)ethyl)pyridin-2-amine

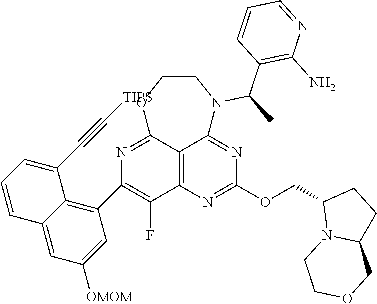

Under nitrogen, a solution of tert-butyl (3-((R)-1-(5-chloro-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-yl)carbamate (500.0 mg, 0.81 mmol), triisopropyl((6-(methoxymethoxy)-8-(4,4,5,5-tetramethyl-1,3,2-dioxaborolan-2-yl)naphthalen-1-yl)ethynyl)silane (415.0 mg, 0.84 mmol), tetrakis(triphenylphosphine)palladium (162.0 mg, 0.14 mmol) and cesium carbonate (684.0 mg, 2.1 mmol) in water (1 mL) and 1,4-dioxane (5 mL) was stirred for 1 hour at 100° C. After completion, the resulting solution was quenched with water and extracted with ethyl acetate. Then the organic layers were combined, washed with brine, dried over anhydrous sodium sulfate and concentrated under vacuum. The residue was purified by flash chromatography on silica gel eluting with dichloromethane/methanol (19:1) to afford 3-((R)-1-(4-fluoro-2-(((6S,8aS)-hexahydro-1H-pyrrolo[2,1-c][1,4]oxazin-6-yl)methoxy)-5-(3-(methoxymethoxy)-8-((triisopropylsilyl)ethynyl)naphthalen-1-yl)-8,9-dihydro-10H-7-oxa-1,3,6,10-tetraazacyclohepta[de]naphthalen-10-yl)ethyl)pyridin-2-amine (320 mg, 0.37 mmol, 45.6% yield) as a yellow solid. LC-MS: (ESI, m/Z):848.4 [M+H]⁺

### Step 4: 3-((R)-1-(5-(8-ethynyl-3-(methoxymethoxy)naphthalen-1-yl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine

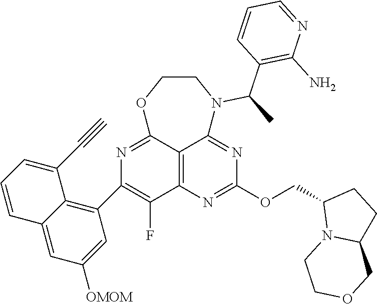

A solution of 3-((R)-1-(4-fluoro-2-(((6S,8aS)-hexahydro-1H-pyrrolo[2,1-c][1,4]oxazin-6-yl)methoxy)-5-(3-(methoxymethoxy)-8-((triisopropylsilyl)ethynyl)naphthalen-1-yl)-8,9-dihydro-10H-7-oxa-1,3,6,10-tetraazacyclohepta[de]naphthalen-10-yl)ethyl)pyridin-2-amine (300.0 mg, 0.35 mmol) and caesium fluoride (304.0 mg, 2 mmol) in N,N-dimethylformamide (3 mL) was stirred at room temperature for 1 hour. After completion, the resulting solution was diluted with water and extracted with ethyl acetate. Then the organic layers were combined, washed with brine, dried over anhydrous sodium sulfate and concentrated under vacuum. The residue was purified by flash chromatography on silica gel eluting with dichloromethane/methanol (47/3) to afford 3-((R)-1-(5-(8-ethynyl-3-(methoxymethoxy)naphthalen-1-yl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine (240.0 mg, 0.35 mmol, 92% yield) as a yellow solid. LC-MS: (ESI, m/Z):692.3 [M+H]⁺

### Step 5: 4-(10-((R)-1-(2-aminopyridin-3-yl)ethyl)-4-fluoro-2-(((6S,8aS)-hexahydro-1H-pyrrolo[2,1-c][1,4]oxazin-6-yl)methoxy)-9,10-dihydro-8H-7-oxa-1,3,6,10-tetraazacyclohepta[de]naphthalen-5-yl)-5-ethynylnaphthalen-2-ol

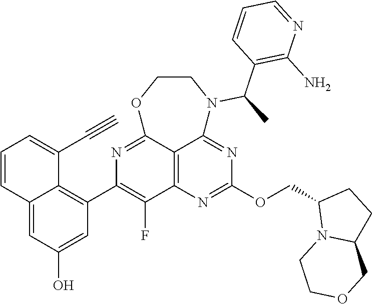 [SMILES: C#Cc1cccc2cc(O)cc(-c3nc4c5c(nc(OC[C@@H]6CC[C@H]7COCCN76)nc5c3F)N([C@H](C)c3cccnc3N)CCO4)c12]

To a solution of 3-((R)-1-(5-(8-ethynyl-3-(methoxymethoxy)naphthalen-1-yl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine (220.0 mg, 0.25 mmol) in acetonitrile (1 mL) was added 4 M hydrochloric acid in dioxane (1 mL), the mixture was stirred at room temperature for 30 minutes. After completion, the solvent was removed under vacuum. The resulting solution was adjusted PH to 7 with saturated sodium bicarbonate solution and extracted with dichloromethane. The organic layers were combined, washed with brine, dried over anhydrous sodium sulfate and concentrated under vacuum. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 32% B to 54% B in 10 min, 54% B; Wave Length: 254/220 nm; RT1(min): 9.6 to afford 4-(10-((R)-1-(2-aminopyridin-3-yl)ethyl)-4-fluoro-2-(((6S,8aS)-hexahydro-1H-pyrrolo[2,1-c][1,4]oxazin-6-yl)methoxy)-9,10-dihydro-8H-7-oxa-1, 3,6,10-tetraazacyclohepta[de]naphthalen-5-yl)-5-ethynylnaphthalen-2-ol (15.3 mg, 0.023 mmol, 9.3% yield). LC-MS: (ESI, m/Z): 648.2 [M+H]⁺

Example 17: ¹H NMR (300 MHz, Methanol-d₄, ppm) δ 7.98-7.96 (m, 1H), 7.81-7.78 (m, 2H), 7.53-7.49 (m, 1H), 7.40-7.38 (m, 1H), 7.33 (d, J=2.6 Hz, 1H), 7.29-7.15 (m, 1H), 6.82-6.77 (m, 1H), 6.64-6.57 (m, 1H), 4.86-4.65 (m, 2H), 4.55-4.41 (m, 2H), 4.02 (s, 1H), 3.92-3.71 (m, 4H), 3.67-3.60 (m, 4H), 3.31-3.14 (m, 2H), 2.44-2.35 (m, 1H), 2.19-2.07 (m, 3H), 1.70 (d, J=7.0 Hz, 3H).

### Example 18: 3-((R)-1-(5-(5-ethynylisoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Synthetic Route

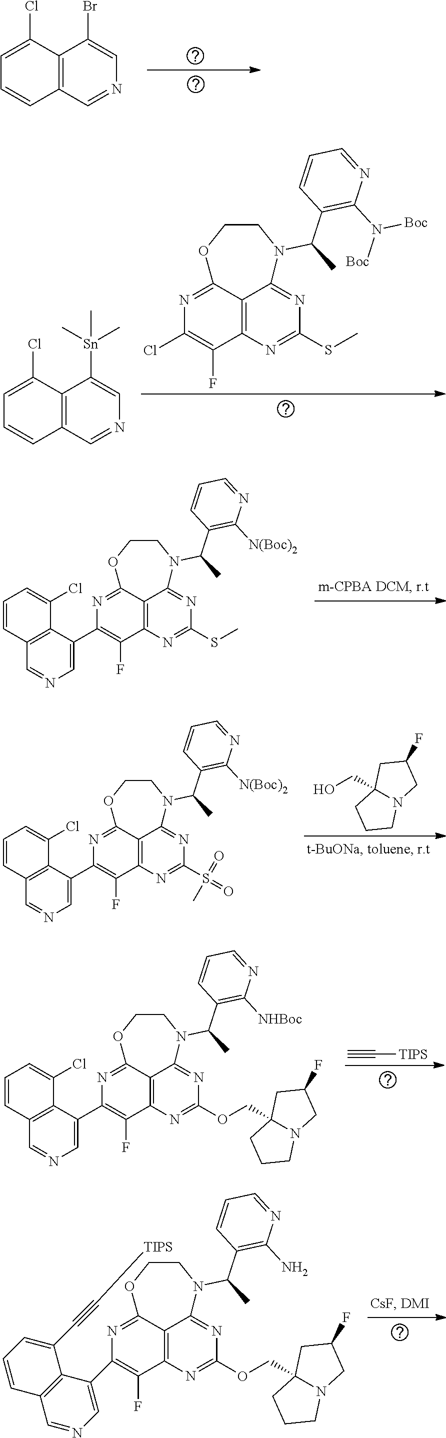

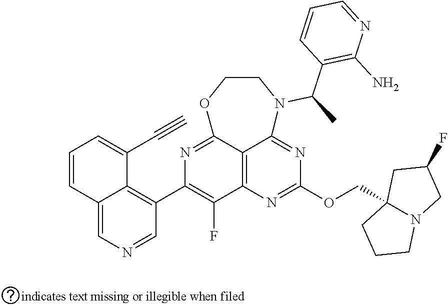 [SMILES: C#Cc1cccc2cncc(-c3nc4c5c(nc(OC[C@@]67CCCN6C[C@H](F)C7)nc5c3F)N([C@H](C)c3cccnc3N)CCO4)c12]

Step 1: 5-chloro-4-(trimethylstannyl)isoquinoline

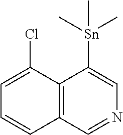

Under nitrogen, a solution of 4-bromo-5-chloroisoquinolinee (1.5 g, 6.19 mmol), hexamethyldistannane (6.1 g, 18.62 mmol) and tetrakis(triphenylphosphine)palladium (0.72 g, 0.62 mmol) in toluene (15 mL) was stirred for 48 hours at 100° C. After completion, the resulting solution was quenched with saturated potassium fluoride solution and extracted with ethyl acetate. Then the organic layers were combined, washed with brine, dried over anhydrous sodium sulfate and concentrated under vacuum. The residue was purified by reverse phase chromatography (acetonitrile/0.1% NH₄HCO₃ in water) to afford 5-chloro-4-(trimethylstannyl)isoquinoline (1.0 g, 3.06 mmol, 49.5% yield) as a colorless oil. LC-MS: (ESI, m/Z): 328.0 [M+H]⁺

### Step 2: tert-butyl N-tert-butoxycarbonyl-N-[3-[(1R)-1-[7-(5-chloro-4-isoquinolyl)-6-fluoro-3-methylsulfanyl-10-oxa-2,4,8,13-tetrazatricyclo[7.4.1.05, 14]tetradeca-1,3,5(14),6,8-pentaen-13-yl]ethyl]-2-pyridyl]carbamate

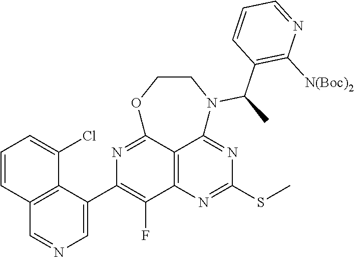

Under nitrogen, a solution of tert-butyl N-tert-butoxycarbonyl-N-[3-[(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl]-2-pyridyl]carbamate (800.0 mg, 1.32 mmol), (5-chloro-4-isoquinolyl)-trimethyl-stannane (860.0 mg, 2.63 mmol), 1.1′-binaphthyl-2.2′-diphemyl phosphine (164.0 mg, 0.26 mmol), cuprous iodide (76.0 mg, 0.40 mmol) and 1,1′-bis(diphenylphosphino)ferrocene-palladium(II)dichloride dichloromethane complex (107.0 mg, 0.13 mmol) in toluene (8 mL) was stirred for 1 hour at 90° C. After completion, the resulting solution was quenched with saturated potassium fluoride solution and extracted with ethyl acetate. Then the organic layers were combined, washed with brine, dried over anhydrous sodium sulfate and concentrated under vacuum. The residue was purified by flash chromatography on silica gel eluting with dichloromethane/methanol (97:3) to afford tert-butyl N-tert-butoxycarbonyl-N-[3-[(1R)-1-[7-(5-chloro-4-isoquinolyl)-6-fluoro-3-methylsulfanyl-10-oxa-2,4,8,13-tetrazatricyclo[7.4.1.05, 14]tetradeca-1,3,5(14),6,8-pentaen-13-yl]ethyl]-2-pyridyl]carbamate (653 mg, 0.89 mmol, 67.5% yield) as a yellow solid. LC-MS: (ESI, m/Z): 734.2 [M+H]⁺

### Step 3: tert-butyl N-tert-butoxycarbonyl-N-[3-[(1R)-1-[7-(5-chloro-4-isoquinolyl)-6-fluoro-3-methylsulfonyl-10-oxa-2,4,8,13-tetrazatricyclo[7.4.1.05, 14]tetradeca-1,3,5(14),6,8-pentaen-13-yl]ethyl]-2-pyridyl]carbamate

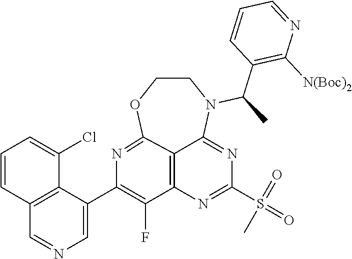

To a solution of tert-butyl N-tert-butoxycarbonyl-N-[3-[(1R)-1-[7-(5-chloro-4-isoquinolyl)-6-fluoro-3-methylsulfanyl-10-oxa-2,4,8,13-tetrazatricyclo[7.4.1.05, 14]tetradeca-1,3,5(14),6,8-pentaen-13-yl]ethyl]-2-pyridyl]carbamate (633.0 mg, 0.86 mmol) in dichloromethane (7 mL) was added 3-chloroperoxybenzoic acid (374.0 mg, 2.17 mmol), the mixture was stirred at room temperature for 0.5 hour. After completion, the resulting solution was quenched with saturated sodium bisulfite solution and extracted with ethyl acetate. Then the organic layers were combined, washed with brine, dried over anhydrous sodium sulfate and concentrated under vacuum. The residue was purified by flash chromatography on silica gel eluting with dichloromethane/methanol (97:3) to afford tert-butyl N-tert-butoxycarbonyl-N-[3-[(1R)-1-[7-(5-chloro-4-isoquinolyl)-6-fluoro-3-methylsulfonyl-10-oxa-2,4,8,13-tetrazatricyclo[7.4.1.05, 14]tetradeca-1,3,5(14),6,8-pentaen-13-yl]ethyl]-2-pyridyl]carbamate (493.0 mg, 0.64 mmol, 74.6% yield) as a yellow solid. LC-MS: (ESI, m/Z): 766.2 [M+H]⁺

### Step 4: tert-butyl (3-((R)-1-(5-(5-chloroisoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate

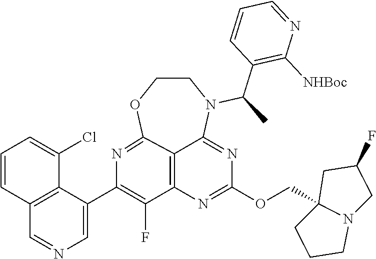 [SMILES: C[C@H](c1cccnc1NC(=O)OC(C)(C)C)N1CCOc2nc(-c3cncc4cccc(Cl)c34)c(F)c3nc(OC[C@@]45CCCN4C[C@H](F)C5)nc1c23]

Under nitrogen, to a solution of ((2R,7aS)-2-fluorotetrahydro-1H-pyrrolizin-7a(5H)-yl)methanol (300.0 mg, 1.88 mmol) in toluene (5 mL) was added sodium tert-butoxide (181.0 mg, 1.88 mmol), the mixture was stirred at 0° C. for 10 minutes. Then tert-butyl N-tert-butoxycarbonyl-N-[3-[(1R)-1-[7-(5-chloro-4-isoquinolyl)-6-fluoro-3-methylsulfonyl-10-oxa-2,4,8,13-tetrazatricyclo[7.4.1.05, 14]tetradeca-1,3,5(14),6,8-pentaen-13-yl]ethyl]-2-pyridyl]carbamate (480.0 mg, 0.63 mmol) was added and stirred at room temperature for 0.5 hour. After completion, the resulting solution was quenched with saturated ammonium chloride solution and extracted with ethyl acetate. Then the organic layers were combined, washed with brine, dried over anhydrous sodium sulfate and concentrated under vacuum. The residue was purified by flash chromatography on silica gel eluting with dichloromethane/methanol (91:9) to afford tert-butyl (3-((R)-1-(5-(5-chloroisoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (170 mg, 0.23 mmol, 36.4% yield) as a yellow solid. LC-MS: (ESI, m/Z): 745.3 [M+H]⁺

### Step 5: 3-((R)-1-(4-fluoro-2-(((2R,7aS)-2-fluorotetrahydro-1H-pyrrolizin-7a(5H)-yl)methoxy)-5-(5-((triisopropylsilyl)ethynyl)isoquinolin-4-yl)-8,9-dihydro-10H-7-oxa-1,3,6,10-tetraazacyclohepta[de]naphthalen-10-yl)ethyl)pyridin-2-amine

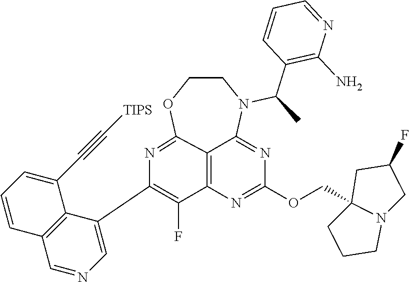

Under nitrogen, a solution of tert-butyl (3-((R)-1-(5-(5-chloroisoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (150.0 mg, 0.18 mmol), ethynyltriisopropylsilane (367.0 mg, 2.01 mmol), bis(acetonitrile)dichloropalladium(II) (6.0 mg, 0.02 mmol), X-phos (58.0 mg, 0.12 mmol) and cesium carbonate (197.0 mg, 0.60 mmol) in acetonitrile (2 mL) was added at 90° C. for 4 hours. After completion, the resulting solution was diluted with water and extracted with ethyl acetate. Then the organic layers were combined, washed with brine, dried over anhydrous sodium sulfate and concentrated under vacuum. The residue was purified by flash chromatography on silica gel eluting with dichloromethane/methanol (89:11) to afford 3-((R)-1-(4-fluoro-2-(((2R,7aS)-2-fluorotetrahydro-1H-pyrrolizin-7a(5H)-yl)methoxy)-5-(5-((triisopropylsilyl)ethynyl)isoquinolin-4-yl)-8,9-dihydro-10H-7-oxa-1,3,6,10-tetraazacyclohepta[de]naphthalen-10-yl)ethyl)pyridin-2-amine (70 mg, 0.09 mmol, 49.1% yield) as a yellow solid. LC-MS: (ESI, m/Z): 791.4 [M+H]⁺

### Step 6: 3-((R)-1-(5-(5-ethynylisoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

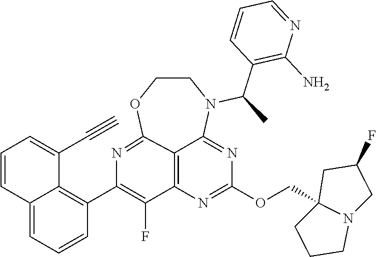 [SMILES: C#Cc1cccc2cccc(-c3nc4c5c(nc(OC[C@@]67CCCN6C[C@H](F)C7)nc5c3F)N([C@H](C)c3cccnc3N)CCO4)c12]

A solution of 3-((R)-1-(4-fluoro-2-(((2R,7aS)-2-fluorotetrahydro-1H-pyrrolizin-7a(5H)-yl)methoxy)-5-(5-((triisopropylsilyl)ethynyl)isoquinolin-4-yl)-8,9-dihydro-10H-7-oxa-1,3,6,10-tetraazacyclohepta[de]naphthalen-10-yl)ethyl)pyridin-2-amine (65.0 mg, 0.08 mmol) and cesium fluoride (39.0 mg, 0.26 mmol) in N,N-dimethylformamide (0.5 mL) was stirred at room temperature for 1.5 hours. After completion, the resulting solution was diluted with water and extracted with ethyl acetate. Then the organic layers were combined, washed with brine, dried over anhydrous sodium sulfate and concentrated under vacuum. The crude product was purified by Prep-HPLC with the following conditions: Column: XBridge Prep OBD C18 Column, 19*250 mm, 5 μm; Mobile Phase A: Water(10 mmol/L NH₄HCO₃), Mobile Phase B: ACN; Flow rate: 25 mL/min; Gradient: 50% B to 80% B in 7 min, 80% B; Wave Length: 254/220 nm; RT1(min): 6.4 to afford 3-((R)-1-(5-(5-ethynylisoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (8.4 mg, 0.01 mmol, 15.7% yield). LC-MS: (ESI, m/Z): 635.3 [M+H]⁺

Example 18: ¹H NMR (300 MHz, Methanol-d₆, ppm) δ9.41 (s, 1H), 8.50 (s, 1H), 8.28 (dd, J=8.1, 1.3 Hz, 1H), 8.04-7.96 (m, 2H), 7.78-7.68 (m, 2H), 6.82-6.76 (m, 1H), 6.62 (q, J=6.7 Hz, 1H), 5.31 (d, J=53.5 Hz, 1H), 4.87-4.36 (m, 4H), 3.49-3.37 (m, 1H), 3.32-3.30 (m, 2H), 3.25-3.17 (m, 3H), 3.03-2.98 (m, 1H), 2.24-2.02 (m, 3H), 2.00-1.95 (m, 3H), 1.69 (d, J=6.6 Hz, 3H).

### Example 19: 3-((R)-1-(5-(5-chloroisoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Synthetic Route

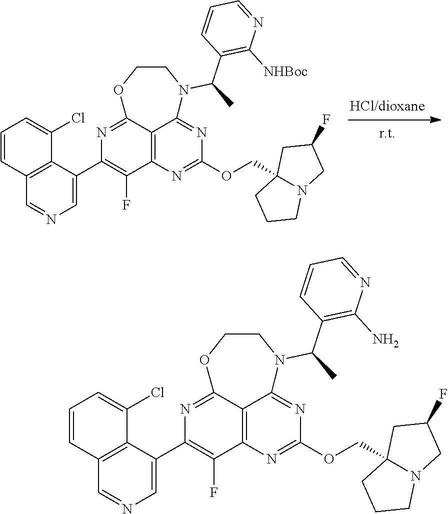 [SMILES: C[C@H](c1cccnc1NC(=O)OC(C)(C)C)N1CCOc2nc(-c3cncc4cccc(Cl)c34)c(F)c3nc(OC[C@@]45CCCN4C[C@H](F)C5)nc1c23 ; C[C@H](c1cccnc1N)N1CCOc2nc(-c3cncc4cccc(Cl)c34)c(F)c3nc(OC[C@@]45CCCN4C[C@H](F)C5)nc1c23]

### Step 1: 3-((R)-1-(5-(5-chloroisoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

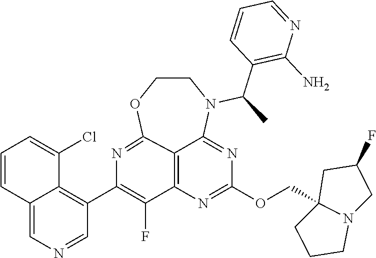 [SMILES: C[C@H](c1cccnc1N)N1CCOc2nc(-c3cncc4cccc(Cl)c34)c(F)c3nc(OC[C@@]45CCCN4C[C@H](F)C5)nc1c23]

A solution of tert-butyl (3-((R)-1-(5-(5-chloroisoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (60.0 mg, 0.07 mmol) in 4 M hydrochloric acid in dioxane (1 mL) was stirred at room temperature for 0.5 hour. After completion, the resulting solution was quenched with saturated sodium bicarbonate solution and extracted with ethyl acetate. Then the organic layers were combined, washed with brine, dried over anhydrous sodium sulfate and concentrated under vacuum. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 28% B to 52% B in 8.5 min; Wave Length: 220 nm; RT1(min): 6.6; Number Of Runs: 2 to afford 3-((R)-1-(5-(5-chloroisoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (13.9 mg, 0.02 mmol, 30.2% yield). LC-MS: (ESI, m/Z): 645.3 [M+H]⁺

Example 19: ¹H NMR (300 MHz, DMSO-d₆, ppm) δ 9.53 (s, 1H), 8.52 (s, 1H), 8.30 (d, J=8.3 Hz, 1H), 7.99-7.92 (m, 2H), 7.80-7.74 (m, 1H), 7.65-7.63 (m, 1H), 6.66 (dd, J=7.5, 4.9 Hz, 1H), 6.36 (q, J=6.8 Hz, 1H), 5.74 (d, J=20.0 Hz, 2H), 5.27 (d, J=54.0 Hz, 1H), 4.46-4.43 (m, 2H), 4.12 (d, J=1.7 Hz, 2H), 3.75-3.71 (m, 1H), 3.54-3.38 (m, 1H), 3.18-2.94 (m, 3H), 2.84-2.82 (m, 1H), 2.22-2.04 (m, 3H), 1.85-1.78 (m, 3H), 1.60-1.56 (m, 3H).

### Example 20: 3-((R)-1-(5-(5-amino-4-fluoro-3-methyl-2-(trifluoromethyl)phenyl)-2-(((S)-4,4-difluoro-1-methylpyrrolidin-2-yl)methoxy)-4-fluoro-8,9-dihydro-10H-7-oxa-1,3,6,10-tetraazacyclohepta[de]naphthalen-10-yl)ethyl)pyridin-2-amine

### Synthetic Route

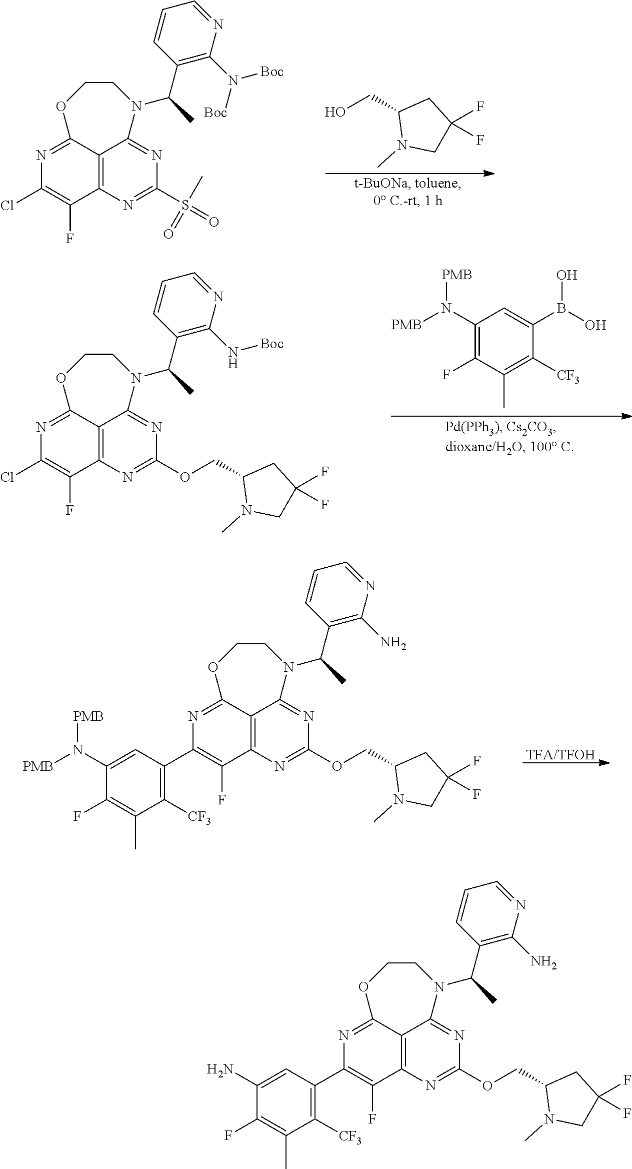

### Step 1: tert-butyl (3 ((R) 1-(5 chloro-2-(((S)-4,4-difluoro-1 methylpyrrolidin-2-yl)methoxy)-4-fluoro-8,9-dihydro-10H-7-oxa-1,3,6,10-tetraazacyclohepta[de]naphthalen-10-yl)ethyl)pyridin-2-yl)carbamate

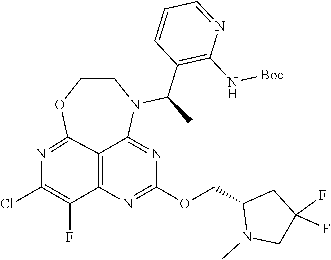 [SMILES: C[C@H](c1cccnc1NC(=O)OC(C)(C)C)N1CCOc2nc(Cl)c(F)c3nc(OC[C@@H]4CC(F)(F)CN4C)nc1c23]

Under nitrogen, to a solution of [(2S)-4,4-difluoro-1-methyl-pyrrolidin-2-yl]methanol (280.0 mg, 1.85 mmol) in toluene (4 mL) was added sodium tert-butoxide (180.0 mg, 1.87 mmol), the resulting solution was stirred for 10 min at 0° C. Then tert-butyl N-tert-butoxycarbonyl-N-[3-[(1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl]-2-pyridyl]carbamate (400.0 mg, 0.63 mmol) was added and stirred at room temperature for 1 hour. After completion, the resulting solution was quenched with saturated ammonium chloride solution and extracted with ethyl acetate. Then the organic layers were combined, washed with brine, dried over anhydrous sodium sulfate and concentrated under vacuum. The residue was purified by flash chromatography on silica gel eluting with dichloromethane/methanol (97:3) to afford tert-butyl (3-((R)-1-(5-chloro-2-(((S)-4,4-difluoro-1-methylpyrrolidin-2-yl)methoxy)-4-fluoro-8,9-dihydro-10H-7-oxa-1,3,6,10-tetraazacyclohepta[de]naphthalen-10-yl)ethyl)pyridin-2-yl)carbamate (320 mg, 0.32 mmol, 52.6% yield) as a yellow solid. LC-MS: (ESI, m/Z): 610.2 [M+H]⁺

### Step 2: 3-((R)-1-(5-(5-(bis(4-methoxybenzyl)amino)-4-fluoro-3-methyl-2-(trifluoromethyl)phenyl)-2-(((S)-4,4-difluoro-1-methylpyrrolidin-2-yl)methoxy)-4-fluoro-8,9-dihydro-10H-7-oxa-1,3,6,10-tetraazacyclohepta[de]naphthalen-10-yl)ethyl)pyridin-2-amine

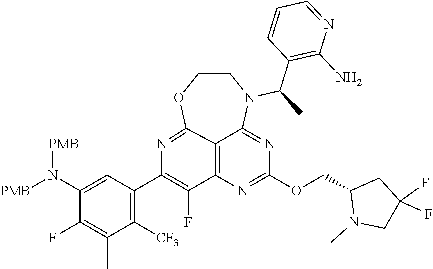

Under nitrogen, a solution of tert-butyl (3-((R)-1-(5-chloro-2-(((S)-4,4-difluoro-1-methylpyrrolidin-2-yl)methoxy)-4-fluoro-8,9-dihydro-10H-7-oxa-1,3,6,10-tetraazacyclohepta[de]naphthalen-10-yl)ethyl)pyridin-2-yl)carbamate (300.0 mg, 0.42 mmol), [5-[bis[(4-methoxyphenyl)methyl]amino]-4-fluoro-3-methyl-2-(trifluoromethyl)phenyl]boronic acid (465.0 mg, 0.97 mmol), tetrakis(triphenylphosphine)palladium (105.0 mg, 0.0900 mmol) and cesium carbonate (420.0 mg, 1.29 mmol) in 1,4-dioxane (3 mL) and water (0.6 mL) was stirred for 2 hours at 100° C. After completion, the resulting solution was diluted with water and extracted with ethyl acetate. Then the organic layers were combined, washed with brine, dried over anhydrous sodium sulfate and concentrated under vacuum. The residue was purified by flash chromatography on silica gel eluting with dichloromethane/methanol (96:4) to afford 3-((R)-1-(5-(5-(bis(4-methoxybenzyl)amino)-4-fluoro-3-methyl-2-(trifluoromethyl)phenyl)-2-(((S)-4,4-difluoro-1-methylpyrrolidin-2-yl)methoxy)-4-fluoro-8,9-dihydro-10H-7-oxa-1,3,6,10-tetraazacyclohepta[de]naphthalen-10-yl)ethyl)pyridin-2-amine (399 mg, 0.36 mmol, 85.4% yield) as a yellow solid. LC-MS: (ESI, m/Z): 907.3 [M+H]⁺

### Step 3: 3-((R)-1-(5-(5-amino-4-fluoro-3-methyl-2-(trifluoromethyl)phenyl)-2-(((S)-4,4-difluoro-1-methylpyrrolidin-2-yl)methoxy)-4-fluoro-8,9-dihydro-10H-7-oxa-1,3,6,10-tetraazacyclohepta[de]naphthalen-10-yl)ethyl)pyridin-2-amine

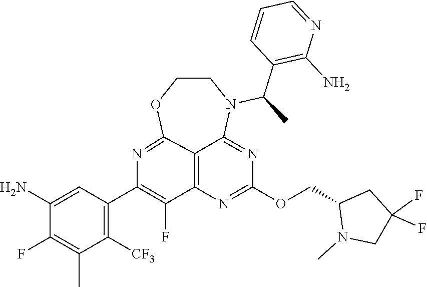 [SMILES: Cc1c(F)c(N)cc(-c2nc3c4c(nc(OC[C@@H]5CC(F)(F)CN5C)nc4c2F)N([C@H](C)c2cccnc2N)CCO3)c1C(F)(F)F]

A solution of 3-((R)-1-(5-(5-(bis(4-methoxybenzyl)amino)-4-fluoro-3-methyl-2-(trifluoromethyl)phenyl)-2-(((S)-4,4-difluoro-1-methylpyrrolidin-2-yl)methoxy)-4-fluoro-8,9-dihydro-10H-7-oxa-1,3,6,10-tetraazacyclohepta[de]naphthalen-10-yl)ethyl)pyridin-2-amine (380.0 mg, 0.34 mmol) in trifluoromethanesulfonic acid (0.4 mL) in 2,2,2-trifluoroacetic acid (4 mL) was stirred at room temperature for 10 min. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 40% B to 55% B in 10 min, 55% B; Wave Length: 254/220 nm; RT (min): 8.9 to afford 3-((R)-1-(5-(5-amino-4-fluoro-3-methyl-2-(trifluoromethyl)phenyl)-2-(((S)-4,4-difluoro-1-methylpyrrolidin-2-yl)methoxy)-4-fluoro-8,9-dihydro-10H-7-oxa-1,3,6,10-tetraazacyclohepta[de]naphthalen-10-yl)ethyl)pyridin-2-amine (55.4 mg, 0.08 mmol, 24% yield). LC-MS: (ESI, m/Z): 667.4[M+H]⁺

Example 20: ¹H NMR (400 MHz, DMSO-d₆, ppm) δ 7.97 (dd, J=4.9, 1.7 Hz, 1H), 7.63 (dd, J=7.5, 1.8 Hz, 1H), 6.67 (dd, J=7.5, 4.9 Hz, 1H), 6.54 (d, J=8.6 Hz, 1H), 6.33 (q, J=6.8 Hz, 1H), 6.02 (s, 2H), 5.65 (s, 2H), 4.46-4.29 (m, 4H), 3.71 (dd, J=15.9, 6.4 Hz, 1H), 3.36-3.34 (m, 1H), 3.01-2.95 (m, 1H), 2.73-2.62 (m, 1H), 2.60-2.53 (m, 1H), 2.48-2.45 (m, 1H), 2.38 (s, 3H), 2.32 (s, 3H), 2.24-2.06 (m, 1H), 1.56 (d, J=6.8 Hz, 3H).

### Example 21a: 3-((R)-1-((R)-5-(3-amino-2-chloro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Example 21b: 3-((R)-1-((S)-5-(3-amino-2-chloro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Synthetic Route

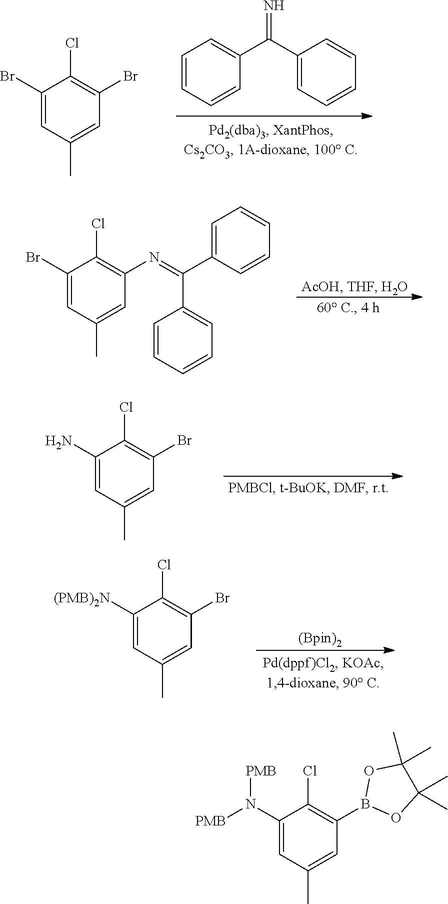

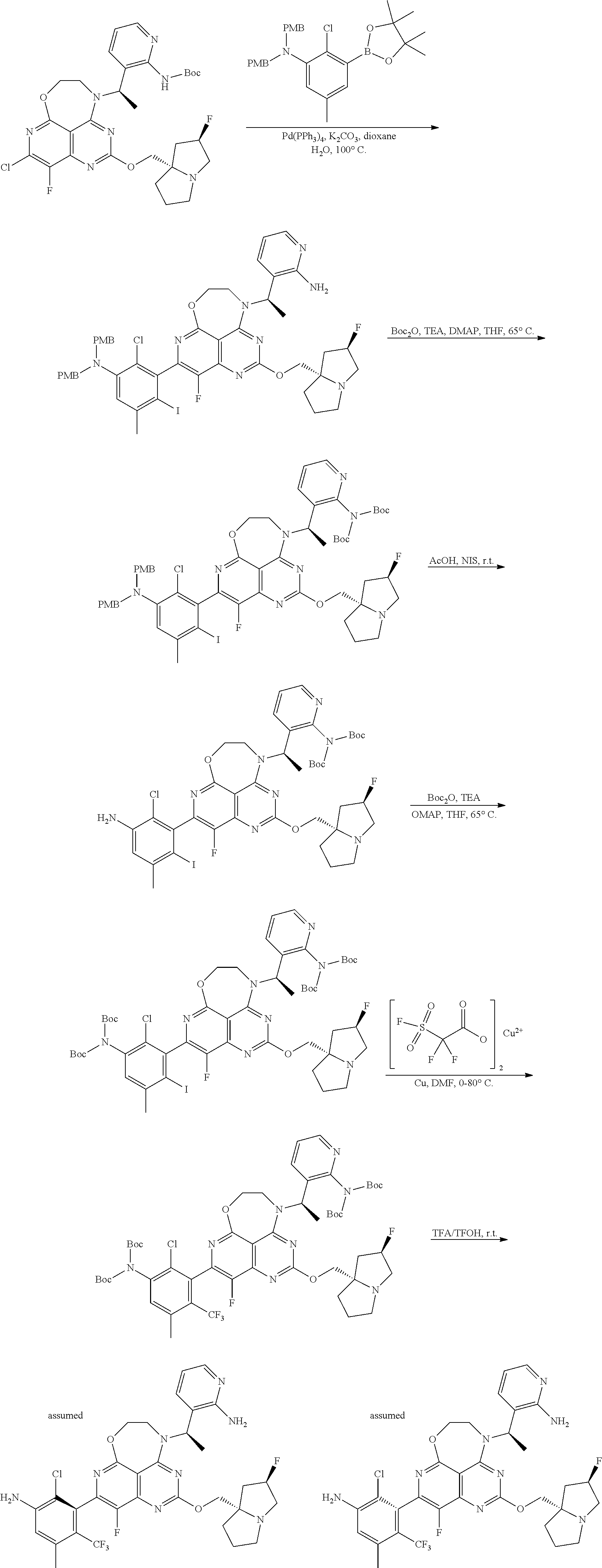

### Step 1: N-(3-bromo-2-chloro-5-methylphenyl)-1,1-diphenylmethanimine

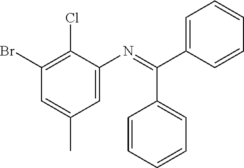 [SMILES: Cc1cc(Br)c(Cl)c(N=C(c2ccccc2)c2ccccc2)c1]

Under nitrogen, a solution of 1,3-dibromo-2-chloro-5-methylbenzene (8.0 g, 28.17 mmol), diphenylmethanimine (7.6 g, 42.25 mmol), tris(dibenzylideneacetone)dipalladium (3.2 g, 2.82 mmol), 4,5-bis(diphenylphosphino)-9,9-dimethylxanthene (3.3 g, 5.63 mmol) and cesium carbonate (27.6 g, 84.51 mmol) in 1,4-dioxane (80 mL) was stirred at 100° C. for 2 hours. After completion, the resulting solution was diluted with water and extracted with ethyl acetate. Then the organic layers were combined, washed with brine, dried over anhydrous sodium sulfate and concentrated under vacuum. The residue was purified by flash chromatography on silica gel eluting with petroleum ether/dichloromethane (23:2) to afford N-(3-bromo-2-chloro-5-methylphenyl)-1,1-diphenylmethanimine (5.0 g, 13.02 mmol, 44.5% yield) as a yellow solid. LC-MS: (ESI, m/Z): 384.0 [M+H]⁺

### Step 2: 3-bromo-2-chloro-5-methylaniline

A solution of N-(3-bromo-2-chloro-5-methylphenyl)-1,1-diphenylmethanimine (5.0 g, 13.00 mmol) in acetic acid (25 mL), tetrahydrofuran (25 mL) and water (5 mL) was stirred at 60° C. for 2 hours. After completion, the solvent was removed under vacuum. The residue was purified by flash chromatography on silica gel eluting with petroleum ether/dichloromethane (23:2) to afford 3-bromo-2-chloro-5-methylaniline (2.0 g, 9.07 mmol, 69.8% yield) as a colorless oil. LC-MS: (ESI, m/Z): 219.9 [M+H]⁺

### Step 3: 3-bromo-2-chloro-N,N-bis(4-methoxybenzyl)-5-methylaniline

To a solution of 3-bromo-2-chloro-5-methyl-aniline (2.0 g, 9.07 mmol) in N,N-dimethylformamide (20 mL) was added potassium tert-butoxide (3.1 g, 27.63 mmol) and 4-methoxybenzylchloride (4.3 g, 27.46 mmol) at 0° C., the mixture was stirred at room temperature for 0.5 hour. After completion, the resulting solution was diluted with water with ethyl acetate. Then the organic layers were combined, washed with brine, dried over anhydrous sodium sulfate and concentrated under vacuum. The residue was purified by flash chromatography on silica gel eluting with petroleum ether/dichloromethane (9:1) to afford 3-bromo-2-chloro-N,N-bis(4-methoxybenzyl)-5-methylaniline (2.6 g, 5.64 mmol, 62.2% yield) as a yellow oil. LC-MS: (ESI, m/Z): 460.1 [M+H]⁺

### Step 4: 2-chloro-N,N-bis(4-methoxybenzyl)-5-methyl-3-(4,4,5,5-tetramethyl-1,3,2-dioxaborolan-2-yl)aniline

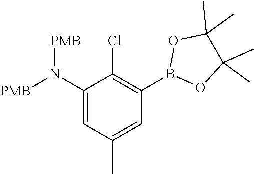

Under nitrogen, a solution of 3-bromo-2-chloro-N,N-bis(4-methoxybenzyl)-5-methylaniline (2.6 g, 5.64 mmol), 4,4,4′,4′,5,5,5′,5′-octamethyl-2,2′-bi(1,3,2-dioxaborolane) (4.3 g, 16.89 mmol), 1,1′-bis(diphenylphosphino)ferrocene-palladium(II)dichloride dichloromethane complex (0.5 g, 0.64 mmol) and potassium acetate (1.7 g, 17.22 mmol) in 1,4-dioxane (26 mL) was added at 90° C. for 3 hours. After completion, the resulting solution was diluted with water and extracted with ethyl acetate. Then the organic layers were combined, washed with brine, dried over anhydrous sodium sulfate and concentrated under vacuum. The resulting residue was purified by reverse phase chromatography (acetonitrile/0.1% NH₄HCO₃ in water) to afford 2-chloro-N,N-bis(4-methoxybenzyl)-5-methyl-3-(4,4,5,5-tetramethyl-1,3,2-dioxaborolan-2-yl)aniline (1.4 g, 2.76 mmol, 48.9% yield) as a yellow solid. LC-MS: (ESI, m/Z): 508.2 [M+H]⁺

### Step 5: 3-((R)-1-(5-(3-(bis(4-methoxybenzyl)amino)-2-chloro-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

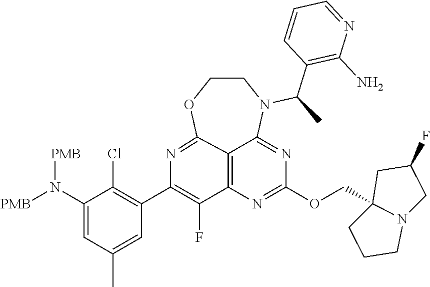

Under nitrogen, a solution of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (1.00 g, 1.62 mmol), 2-chloro-N,N-bis(4-methoxybenzyl)-5-methyl-3-(4,4,5,5-tetramethyl-1,3,2-dioxaborolan-2-yl)aniline (1.2 g, 2.43 mmol), tetrakis(triphenylphosphine)palladium (0.4 g, 0.32 mmol) and potassium carbonate (0.67 g, 4.85 mmol) in 1,4-dioxane (10 mL) and water (2 mL) was stirred at 100° C. for 1 hour. After completion, the resulting solution was diluted with water and extracted with ethyl acetate. Then the organic layers were combined, washed with brine, dried over anhydrous sodium sulfate and concentrated under vacuum. The residue was purified by flash chromatography on silica gel eluting with dichloromethane/methanol (24:1) to afford 3-((R)-1-(5-(3-(bis(4-methoxybenzyl)amino)-2-chloro-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (602.0 mg, 0.70 mmol, 43.1% yield) as a yellow solid. LC-MS: (ESI, m/Z): 863.4 [M+H]⁺

### Step 6:tert-butyl N-[3-[(1R)-1-[7-[3-[bis[(4-methoxyphenyl)methyl]amino]-2-chloro-5-methyl-phenyl]-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate

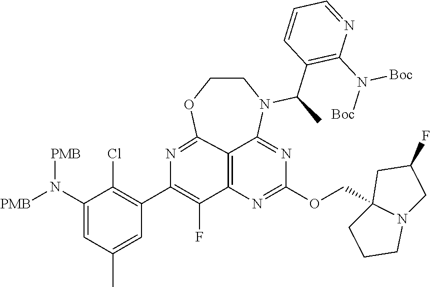

A solution of 3-((R)-1-(5-(3-(bis(4-methoxybenzyl)amino)-2-chloro-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (1.0 g, 1.16 mmol), di-tert-butyl dicarbonate (1.30 g, 5.77 mmol), 4-dimethylaminopyridine (30.0 mg, 0.25 mmol) and triethylamine (590 mg, 5.83 mmol) in tetrahydrofuran (10 ml) was stirred at 65° C. for 1 hour. After completion, the resulting solution was diluted with water and extracted with ethyl acetate. Then the organic layers were combined, washed with brine, dried over anhydrous sodium sulfate and concentrated under vacuum. The residue was purified by flash chromatography on silica gel eluting with dichloromethane/methanol (93:7) to afford tert-butyl N-[3-[(1R)-1-[7-[3-[bis[(4-methoxyphenyl)methyl]amino]-2-chloro-5-methyl-phenyl]-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (602.0 mg, 0.57 mmol, 48.9% yield) as a yellow solid. LC-MS: (ESI, m/Z): 1063.5 [M+H]⁺

### Step 7: tert-butyl N-[3-[(1R)-1-[7-(3-amino-2-chloro-6-iodo-5-methyl-phenyl)-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate

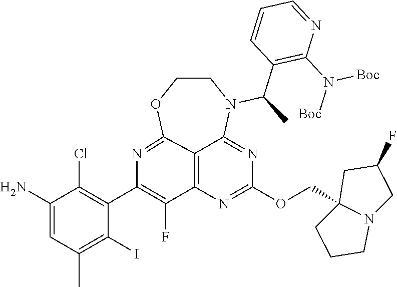 [SMILES: Cc1cc(N)c(Cl)c(-c2nc3c4c(nc(OC[C@@]56CCCN5C[C@H](F)C6)nc4c2F)N([C@H](C)c2cccnc2N(C(=O)OC(C)(C)C)C(=O)OC(C)(C)C)CCO3)c1I]

To a solution of tert-butyl N-[3-[(1R)-1-[7-[3-[bis[(4-methoxyphenyl)methyl]amino]-2-chloro-5-methyl-phenyl]-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (600.0 mg, 0.56 mmol) in acetonitrile (3 mL) and acetic acid (3 mL) was added N-iodosuccinimide (635.0 mg, 2.82 mmol), the mixture was stirred at room temperature for 30 minutes. After completion, the resulting solution was quenched with saturated sodium thiosulfate solution and extracted with ethyl acetate. Then the organic layers were combined, washed with brine, dried over anhydrous sodium sulfate and concentrated under vacuum. The residue was purified by flash chromatography on silica gel eluting with dichloromethane/methanol (19:1) to afford tert-butyl N-[3-[(1R)-1-[7-(3-amino-2-chloro-6-iodo-5-methyl-phenyl)-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (320 mg, 0.34 mmol, 59.8% yield) as a yellow solid. LC-MS: (ESI, m/Z): 949.2 [M+H]⁺

### Step 8: tert-butyl (3-(10-((R)-1-(2-(bis(tert-butoxycarbonyl)amino)pyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-2-chloro-4-iodo-5-methylphenyl)(tert-butoxycarbonyl)carbamate

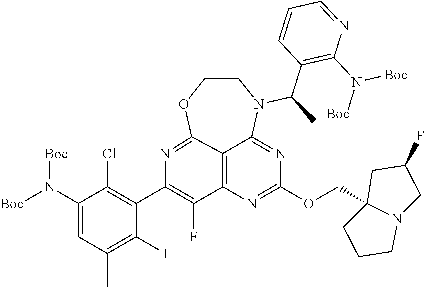 [SMILES: Cc1cc(N(C(=O)OC(C)(C)C)C(=O)OC(C)(C)C)c(Cl)c(-c2nc3c4c(nc(OC[C@@]56CCCN5C[C@H](F)C6)nc4c2F)N([C@H](C)c2cccnc2N(C(=O)OC(C)(C)C)C(=O)OC(C)(C)C)CCO3)c1I]

A solution of tert-butyl N-[3-[(1R)-1-[7-(3-amino-2-chloro-6-iodo-5-methyl-phenyl)-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (220.0 mg, 0.23 mmol), 4-dimethylaminopyridine (6.0 mg, 0.05 mmol), di-tert-butyl dicarbonate (506.0 mg, 2.32 mmol) and triethylamine (265.0 mg, 2.62 mmol) in tetrahydrofuran (2.5 mL) was stirred at 65° C. for 1 hour. After completion, the resulting solution was diluted with water and extracted with ethyl acetate. Then the organic layers were combined, washed with brine, dried over anhydrous sodium sulfate and concentrated under vacuum. The residue was purified by flash chromatography on silica gel eluting with dichloromethane/methanol (91:9) to afford tert-butyl (3-(10-((R)-1-(2-(bis(tert-butoxycarbonyl)amino)pyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-2-chloro-4-iodo-5-methylphenyl)(tert-butoxycarbonyl)carbamate (182.0 mg,0.16 mmol, 68.3% yield) as a yellow solid. LC-MS: (ESI, m/Z): 1049.3 [M+H]⁺

### Step 9:tert-butyl (3-(10-((R)-1-(2-(bis(tert-butoxycarbonyl)amino)pyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-2-chloro-5-methyl-4-(trifluoromethyl)phenyl)(tert-butoxycarbonyl)carbamate

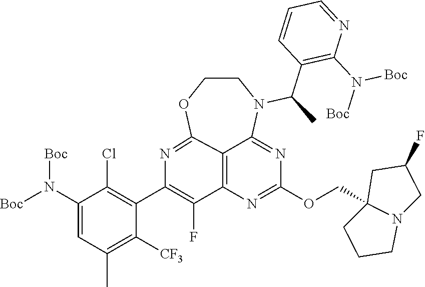 [SMILES: Cc1cc(N(C(=O)OC(C)(C)C)C(=O)OC(C)(C)C)c(Cl)c(-c2nc3c4c(nc(OC[C@@]56CCCN5C[C@H](F)C6)nc4c2F)N([C@H](C)c2cccnc2N(C(=O)OC(C)(C)C)C(=O)OC(C)(C)C)CCO3)c1C(F)(F)F]

Under nitrogen, to a solution of tert-butyl (3-(10-((R)-1-(2-(bis(tert-butoxycarbonyl)amino)pyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-2-chloro-4-iodo-5-methylphenyl)(tert-butoxycarbonyl)carbamate (170.0 mg, 0.15 mmol) and copper (102.0 mg, 1.61 mmol) in N,N-dimethylformamide (2 mL) was added bis[(2,2-difluoro-2-fluorosulfonyl-acetyl)oxy]copper (612.0 mg, 1.46 mmol) and stirred at −78° C. for 10 minutes, then the mixture was removed to 80° C. and stirred for 1 hour. The residue was purified by flash chromatography on silica gel eluting with dichloromethane/methanol (91:9) to afford tert-butyl (3-(10-((R)-1-(2-(bis(tert-butoxycarbonyl)amino)pyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-2-chloro-5-methyl-4-(trifluoromethyl)phenyl)(tert-butoxycarbonyl)carbamate (130.0 mg, 0.10 mmol, 80.5% yield) as a yellow solid. LC-MS: (ESI, m/Z): 1091.4 [M+H]⁺

### Step 10: 3-((R)-1-((R)-5-(3-amino-2-chloro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine & 3-((R)-1-((S)-5-(3-amino-2-chloro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

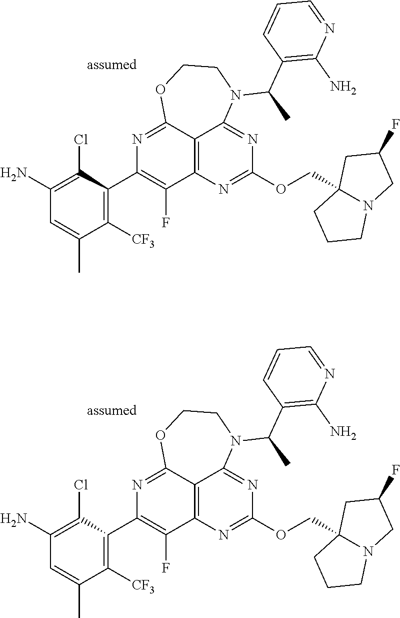

A solution of tert-butyl (3-(10-((R)-1-(2-(bis(tert-butoxycarbonyl)amino)pyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-2-chloro-5-methyl-4-(trifluoromethyl) phenyl)(tert-butoxycarbonyl)carbamate (110.0 mg, 0.10 mmol) in trifluoroacetic acid (1 mL) and trifuoromethanesulfonic acid (0.1 mL) was stirred at room temperature for 0.5 hour. After completion, the resulting solution was quenched with saturated sodium bicarbonate solution and extracted with dichloromethane. Then the organic layers were combined, washed with brine, dried over anhydrous sodium sulfate and concentrated under vacuum. The crude product was purified by Prep-HPLC with the following conditions:Column: Xselect CSH C18 OBD Column 30*150 mm 5 μm, n; Mobile Phase A: Water(0.1% FA), Mobile Phase B: ACN; Flow rate: 60 mL/min; Gradient: 9% B to 21% B in 10 min, 21% B; Wave Length: 254/220 nm; RT (min): 8, 10 to afford 3-((R)-1-((R)-5-(3-amino-2-chloro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (16.4 mg, 0.02 mmol, 21.5% yield) as a white solid and 3-((R)-1-((S)-5-(3-amino-2-chloro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (14.1 mg, 0.02 mmol, 19.6% yield). The stereo chemistry of title compounds was arbitrarily assigned.

Example 21a: 1H NMR (300 MHz,Methanol-d₆, ppm) δ57.96 (s, 1H), 7.75 (d, J=7.5 Hz, 1H), 6.87 (s, 1H), 6.81-6.76 (m, 1H), 6.62 (q, J=6.8 Hz, 1H), 5.40 (d, J=53.2 Hz, 1H), 4.89-4.48 (m, 3H), 4.32 (dd, J=12.8, 6.5 Hz, 1H), 3.76 (dd, J=16.0, 6.6 Hz, 1H), 3.60-3.58 (m, 1H), 3.55-3.36 (m, 3H), 3.18-3.09 (m, 1H), 2.47-2.45 (m, 1H), 2.42-2.39 (m, 3H), 2.37-2.29 (m, 1H), 2.25-2.19 (m, 1H), 2.12-2.04 (m, 3H), 1.64 (d, J=6.9 Hz, 3H). LC-MS: (ESI, m/Z): 691.3 [M+H]⁺.

Example 21b: ¹H NMR (300 MHz, Methanol-d₆, ppm) δ 7.96 (s, 1H), 7.75 (d, J=7.4 Hz, 1H), 6.87 (s, 1H), 6.79-6.76 (m, 1H), 6.63 (q, J=6.8 Hz, 1H), 5.36 (d, J=53.7 Hz, 1H), 4.46 (dd, J=12.8, 6.2 Hz, 1H), 4.43-4.31 (m, 3H), 3.76 (dd, J=16.0, 6.4 Hz, 1H), 3.54 (dd, J=16.0, 6.3 Hz, 1H), 3.37-3.31 (m, 1H), 3.30-3.24 (m, 2H), 3.13-3.00 (m, 1H), 2.41-2.38 (m, 3H), 2.32-2.18 (m, 3H), 2.06-1.98 (m, 3H), 1.67 (d, J=6.9 Hz, 3H). LC-MS: (ESI, m/Z): 691.3 [M+H]⁺.

### Example 22: 3-((1R)-1-(5-(3-amino-2-fluoro-5-methyl-6-(trifluoromethyl)phenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine

### Synthetic Route

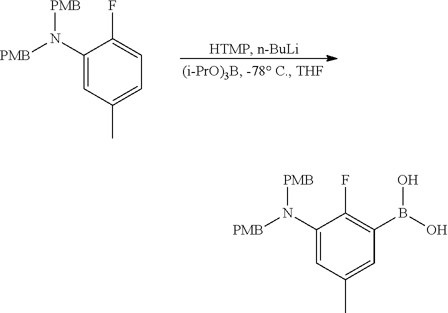

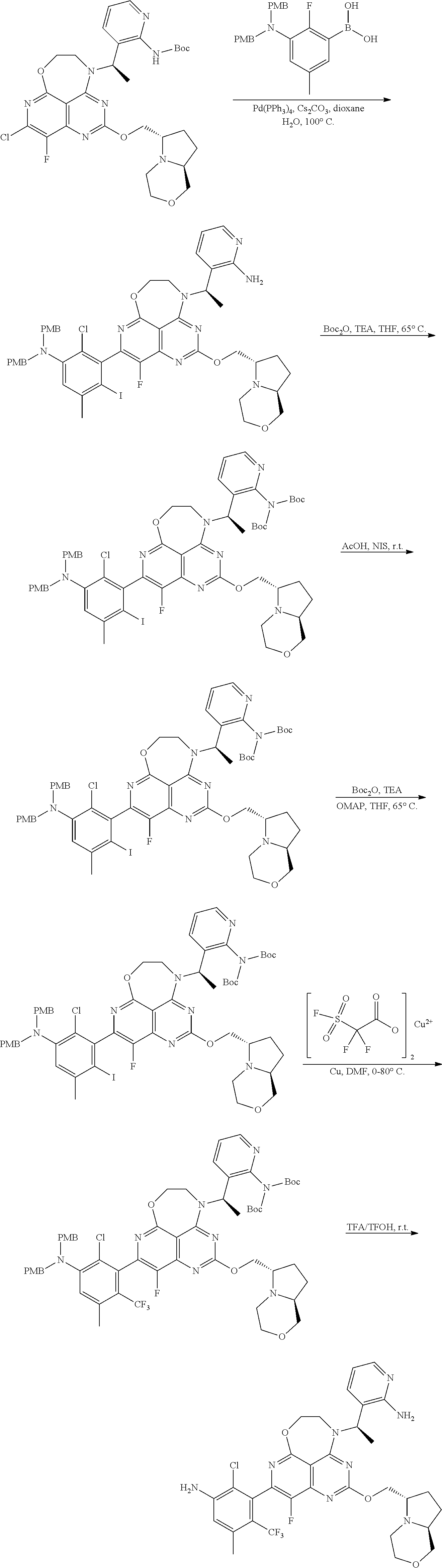

### Step 1: (3-(bis(4-methoxybenzyl)amino)-2-fluoro-5-methylphenyl)boronic acid

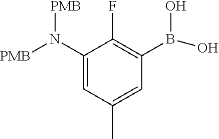

Under nitrogen, to a solution of 2-fluoro-N,N-bis(4-methoxybenzyl)-5-methylaniline (1.0 g, 2.74 mmol) and 2,2,6,6-tetramethylpiperidine (0.58 g, 4.1 mmol) in tetrahydrofuran (10 mL) was added n-butyllithium (3.3 mL, 8.21 mmol) dropwise at −78° C. The resulting solution was stirred for 0.5 hour at −78° C. Then triisopropyl borate (1.54 g, 8.21 mmol) was added and stirred at −78° C. for 1 hour. After completion, the resulting solution was quenched with saturated ammonium chloride solution and extracted with ethyl acetate. Then the organic layers were combined, washed with brine, dried over anhydrous sodium sulfate and concentrated under vacuum. The residue was purified by flash chromatography on silica gel eluting with petroleum ether/ethyl acetate (5:1) to afford (3-(bis(4-methoxybenzyl)amino)-2-fluoro-5-methylphenyl)boronic acid (900 mg, 1.90 mmol, 69.4% yield) as a yellow solid. LC-MS: (ESI, m/Z): 410.2 [M+H]⁺

### Step 2: 3-((R)-1-(5-(3-(bis(4-methoxybenzyl)amino)-2-fluoro-5-methylphenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine

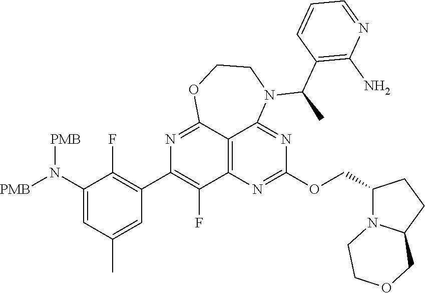

Under nitrogen, a solution of tert-butyl (3-((R)-1-(5-chloro-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-yl)carbamate (500.0 mg, 0.81 mmol), (3-(bis(4-methoxybenzyl)amino)-2-fluoro-5-methylphenyl)boronic acid (675.0 mg, 1.65 mmol), tetrakis(triphenylphosphine)palladium (200.0 mg, 0.17 mmol) and cesium carbonate (800.0 mg, 2.46 mmol) in 1,4-dioxane (5 mL) and water (1 mL) was stirred at 100° C. for 1 hour. After completion, the resulting solution was diluted with water and extracted with ethyl acetate. Then the organic layers were combined, washed with brine, dried over anhydrous sodium sulfate and concentrated under vacuum. The residue was purified by flash chromatography on silica gel eluting with dichloromethane/methanol (24:1) to afford 3-((R)-1-(5-(3-(bis(4-methoxybenzyl)amino)-2-fluoro-5-methylphenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine (250 mg, 0.27 mmol, 33.4% yield) as a yellow solid. LC-MS: (ESI, m/Z): 845.39 [M+H]⁺

### Step 3: tert-butyl N-[3-[(1R)-1-[3-[[(6S,8aS)-3,4,6,7,8,8a-hexahydro-1H-pyrrolo[2,1-c][1,4]oxazin-6-yl]methoxy]-7-[3-[bis[(4-methoxyphenyl)methyl]amino]-2-fluoro-5-methyl-phenyl]-6-fluoro-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate

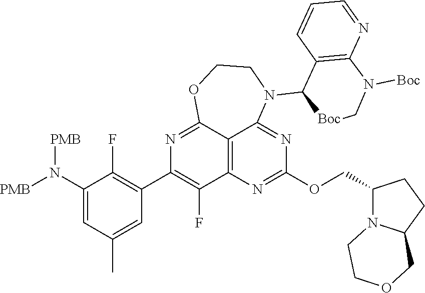

A solution of 3-((R)-1-(5-(3-(bis(4-methoxybenzyl)amino)-2-fluoro-5-methylphenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine (450.0 mg, 0.53 mmol), 4-dimethylaminopyridine (13.0 mg, 0.11 mmol, di-tert-butyl dicarbonate (581.0 mg, 2.66 mmol) and triethylamine (270.0 mg, 2.67 mmol) in tetrahydrofuran (5 mL) was stirred at 65° C. for 1 hour. After completion, the resulting solution was diluted with water and extracted with ethyl acetate. Then the organic layers were combined, washed with brine, dried over anhydrous sodium sulfate and concentrated under vacuum. The residue was purified by flash chromatography on silica gel eluting with dichloromethane/methanol (24:1) to afford tert-butyl N-[3-[(1R)-1-[3-[[(6S,8aS)-3,4,6,7,8,8a-hexahydro-1H-pyrrolo[2,1-c][1,4]oxazin-6-yl]methoxy]-7-[3-[bis[(4-methoxyphenyl)methyl]amino]-2-fluoro-5-methyl-phenyl]-6-fluoro-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (480 mg, 0.41 mmol, 77.6% yield) as a yellow solid. LC-MS: (ESI, m/Z): 1045.5 [M+H]⁺

### Step 4: tert-butyl N-[3-[(1R)-1-[3-[[(6S,8aS)-3,4,6,7,8,8a-hexahydro-1H-pyrrolo[2,1-c][1,4]oxazin-6-yl]methoxy]-7-[3-[bis[(4-methoxyphenyl)methyl]amino]-2-fluoro-6-iodo-5-methyl-phenyl]-6-fluoro-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate

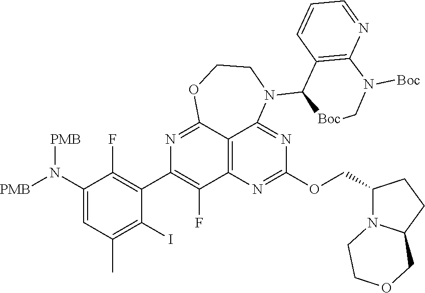

To a solution of tert-butyl N-[3-[(1R)-1-[3-[[(6S,8aS)-3,4,6,7,8,8a-hexahydro-1H-pyrrolo[2,1-c][1,4]oxazin-6-yl]methoxy]-7-[3-[bis[(4-methoxyphenyl)methyl]amino]-2-fluoro-5-methyl-phenyl]-6-fluoro-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (400.0 mg, 0.38 mmol) in acetic acid (2 mL) and acetonitrile (2 mL) was added N-iodosuccinimide (129.0 mg, 0.57 mmol), the mixture was stirred at room temperature for 30 minutes. After completion, the resulting solution was quenched with saturated sodium thiosulfate solution and extracted with ethyl acetate. Then the organic layers were combined, washed with brine, dried over anhydrous sodium sulfate and concentrated under vacuum. The residue was purified by flash chromatography on silica gel eluting with dichloromethane/methanol (19:1) to afford tert-butyl N-[3-[(1R)-1-[3-[[(6S,8aS)-3,4,6,7,8,8a-hexahydro-1H-pyrrolo[2,1-c][1,4]oxazin-6-yl]methoxy]-7-[3-[bis[(4-methoxyphenyl)methyl]amino]-2-fluoro-6-iodo-5-methyl-phenyl]-6-fluoro-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (380 mg, 0.31 mmol, 80% yield) as a yellow solid. LC-MS: (ESI, m/Z): 1171.4 [M+H]⁺

### Step 5: tert-butyl N-[3-[(1R)-1-[3-[[(6S,8aS)-3,4,6,7,8,8a-hexahydro-1H-pyrrolo[2,1-c][1,4]oxazin-6-yl]methoxy]-7-[3-[bis[(4-methoxyphenyl)methyl]amino]-2-fluoro-5-methyl-6-(trifluoromethyl)phenyl]-6-fluoro-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate

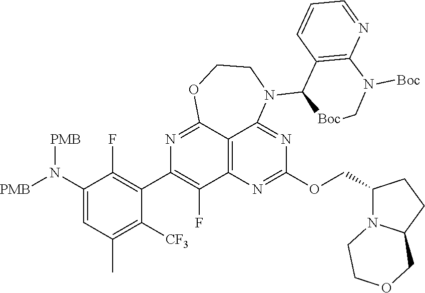

Under nitrogen, to a solution of tert-butyl N-[3-[(1R)-1-[3-[[(6S,8aS)-3,4,6,7,8,8a-hexahydro-1H-pyrrolo[2,1-c][1,4]oxazin-6-yl]methoxy]-7-[3-[bis[(4-methoxyphenyl)methyl]amino]-2-fluoro-6-iodo-5-methyl-phenyl]-6-fluoro-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (260.0 mg, 0.22 mmol) and copper (142.0 mg, 2.23 mmol) in N,N-dimethylformamide (2 mL) was added bis[(2,2-difluoro-2-fluorosulfonyl-acetyl)oxy]copper (929.0 mg, 2.22 mmol) and stirred at −78° C. for 10 minutes, then the mixture was removed to 80° C. and stirred for 1 hour. After completion, the resulting solution was quenched with water and extracted with ethyl acetate. Then the organic layers were combined, washed with brine, dried over anhydrous sodium sulfate and concentrated under vacuum. The residue was purified by flash chromatography on silica gel eluting with dichloromethane/methanol (23:2) to afford tert-butyl N-[3-[(1R)-1-[3-[[(6S,8aS)-3,4,6,7,8,8a-hexahydro-1H-pyrrolo[2,1-c][1,4]oxazin-6-yl]methoxy]-7-[3-[bis[(4-methoxyphenyl)methyl]amino]-2-fluoro-5-methyl-6-(trifluoromethyl)phenyl]-6-fluoro-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (150 mg, 0.10 mmol, 46% yield) as a yellow solid. LC-MS: (ESI, m/Z): 1113.5 [M+H]⁺

### Step 6: 3-((1R)-1-(5-(3-amino-2-fluoro-5-methyl-6-(trifluoromethyl)phenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine

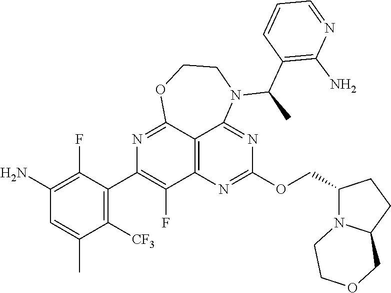 [SMILES: Cc1cc(N)c(F)c(-c2nc3c4c(nc(OC[C@@H]5CC[C@H]6COCCN65)nc4c2F)N([C@H](C)c2cccnc2N)CCO3)c1C(F)(F)F]

A solution of tert-butyl N-[3-[(1R)-1-[3-[[(6S,8aS)-3,4,6,7,8,8a-hexahydro-1H-pyrrolo[2,1-c][1,4]oxazin-6-yl]methoxy]-7-[3-[bis[(4-methoxyphenyl)methyl]amino]-2-fluoro-5-methyl-6-(trifluoromethyl)phenyl]-6-fluoro-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (200.0 mg, 0.18 mmol) in trifluoroacetic acid (2 mL) and trifuoromethanesukfonic acid (0.2 mL) was stirred at room temperature for 20 minutes. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. After completion, the resulting solution was concentrated under vacuum. The crude product was purified by Prep-HPLC with the following conditions:Column: Xselect CSH C18 OBD Column 30*150 mm 5 μm, n; Mobile Phase A: Water(0.1% FA), Mobile Phase B: ACN; Flow rate: 60 mL/min; Gradient: 8% B to 23% B in 9 min, 23% B; Wave Length: 254/220 nm; RT1(min): 8, 9 to afford 3-((1R)-1-(5-(3-amino-2-fluoro-5-methyl-6-(trifluoromethyl)phenyl)-4-fluoro-2-(((6S,8aS)-hexahydro-1H-pyrrolo[2,1-c][1,4]oxazin-6-yl)methoxy)-8,9-dihydro-10H-7-oxa-1,3,6,10-tetraazacyclohepta[de]naphthalen-10-yl)ethyl)pyridin-2-amine (27.8 mg, 0.04 mmol, 22.4% yield). LC-MS: (ESI, m/Z): 673.2 [M+H]⁺

Example 22: ¹H NMR (300 MHz, DMSO-d₆, ppm) δ 7.96 (d, J=4.8 Hz, 1H), 7.61 (dd, J=7.5, 1.7 Hz, 1H), 6.80 (d, J=8.8 Hz, 1H), 6.66 (dd, J=7.5, 4.9 Hz, 1H), 6.33 (q, J=6.8 Hz, 1H), 5.96 (s, 2H), 5.65 (s, 2H), 4.45-4.38 (m, 2H), 4.34-4.25 (m, 2H), 3.71-3.68 (m, 1H), 3.63-3.50 (m, 2H), 3.49-3.46 (m, 1H), 3.44-3.37 (m, 2H), 3.14 (t, J=10.3 Hz, 1H), 3.00-2.97 (m, 1H), 2.90-2.86 (m, 2H), 2.52-2.49 (m, 3H), 2.16-1.97 (m, 1H), 1.84-1.68 (m, 1H), 1.68-1.46 (m, 4H), 1.43-1.28 (m, 1H).

### Example 23: 3-((1R)-1-(5-(3-amino-2-fluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine

### Synthetic Route

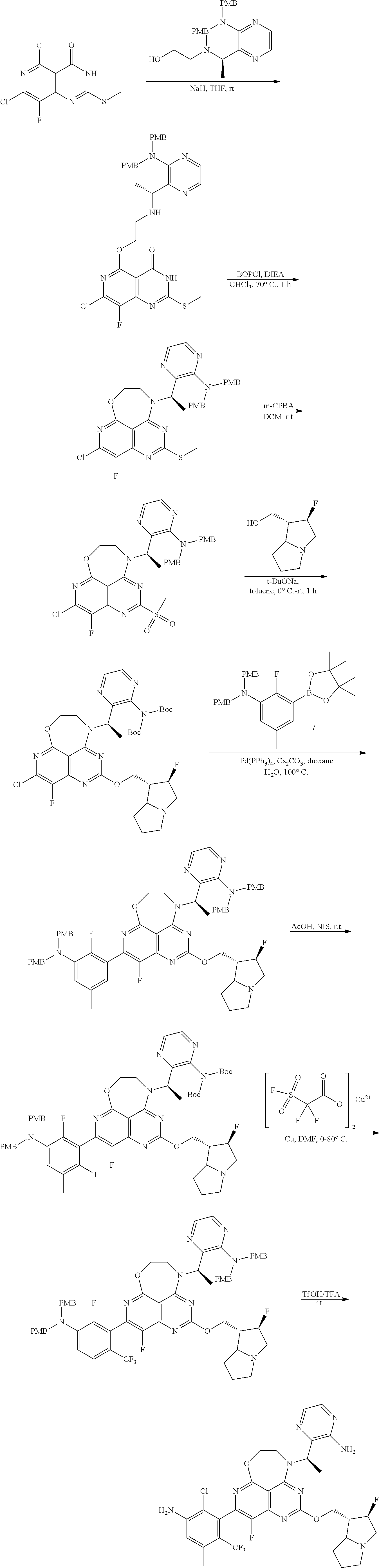

### Step 1: (R)-5-(2-((1-(3-(bis(4-methoxybenzyl)amino)pyrazin-2-yl)ethyl)amino)ethoxy)-7-chloro-8-fluoro-2-(methylthio)pyrido[4,3-d]pyrimidin-4(3H)-one

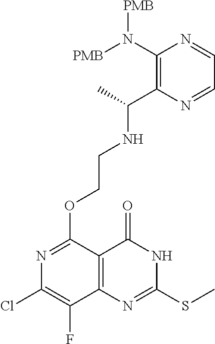

Under nitrogen, to a solution of 2-[[(1R)-1-[3-[bis[(4-methoxyphenyl)methyl]amino]pyrazin-2-yl]ethyl]amino]ethanol (1.13 g, 2.7 mmol) in tetrahydrofuran (9.0 mL) was added sodium hydride (286.0 mg, 7.1 mmol, 60% in mineral oil), the mixture was stirred at 0° C. for 10 minutes. Then the mixture was added into a solution of 5,7-dichloro-8-fluoro-2-methylsulfanyl-3H-pyrido[4,3-d]pyrimidin-4-one (500 mg, 1.8 mmol) in tetrahydrofuran (9.0 mL) and stirred at room temperature for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The crude product would be directly used in the next step without purification. LC-MS: (ESI, m/Z): 666.2 [M+H]⁺.

### Step 2: (R)-3-(1-(5-chloro-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyrazin-2-amine

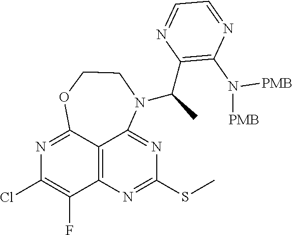

A solution of (R)-5-(2-((1-(3-(bis(4-methoxybenzyl)amino)pyrazin-2-yl)ethyl)amino)ethoxy)-7-chloro-8-fluoro-2-(methylthio)pyrido[4,3-d]pyrimidin-4(3H)-one (2.8 g, crude), bis(2-oxo-3-oxazolidinyl)phosphinic chloride (2.20 g, 8.65 mmol) and N,N-diisopropylethylamine (3.7 mL, 21.62 mmol) in chloroform (28 mL) was stirred at 70° C. for 1 hour. After completion, the resulting solution was diluted with water and extracted with dichloromethane. The organic layers were washed with brine, dried over anhydrous sodium sulfate and concentrated. The residue was purified by flash chromatography on silica gel eluting with petroleum ether/ethyl acetate (1:1) to afford (R)-3-(1-(5-chloro-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyrazin-2-amine (1.5 g, 2.38 mmol, 55.1% yield) as a white solid. LC-MS: (ESI, m/Z): 648.2 [M+H]⁺.

### Step 3: (R)-3-(1-(5-chloro-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyrazin-2-amine

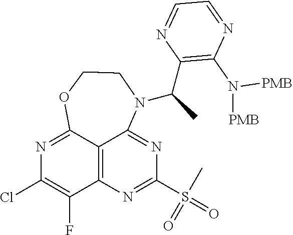

To a solution of (R)-3-(1-(5-chloro-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyrazin-2-amine (1.5 g, 2.31 mmol) in dichloromethane (15 mL) was added 3-chloroperoxybenzoic acid (998.4 mg, 5.79 mmol), the mixture was stirred at 25° C. for 1 hour. After completion, the reaction was quenched with saturated sodium sulfite solution. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1:1) to afford (R)-3-(1-(5-chloro-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyrazin-2-amine (1.0 g, 1.42 mmol, 61.8% yield) as a white solid. LC-MS: (ESI, m/Z): 680.2 [M+H]⁺.

### Step 4: 3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine

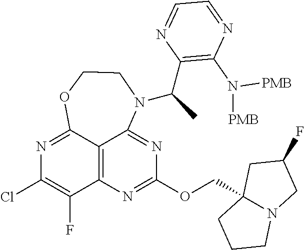

Under nitrogen, to a solution of ((2R,7aS)-2-fluorotetrahydro-1H-pyrrolizin-7a(5H)-yl)methanol (702.2 mg, 4.41 mmol) in toluene (10 mL) was added sodium tert-butoxide (423.8 mg, 4.41 mmol), the mixture was stirred at 0° C. for 10 minutes. Then (R)-3-(1-(5-chloro-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyrazin-2-amine (1.0 g, 1.47 mmol) was added and stirred at 25° C. for 1 hour. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (8:1) to afford 3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine (630.0 mg, 0.79 mmol, 54% yield) as a yellow solid. LC-MS: (ESI, m/Z): 759.3 [M+H]⁺.

### Step 5: 3-((R)-1-(5-(3-(bis(4-methoxybenzyl)amino)-2-fluoro-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine

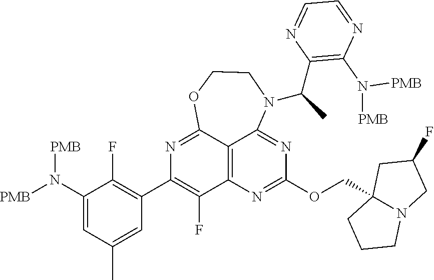

Under nitrogen, a solution of 3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine (500.0 mg, 0.66 mmol), 2-fluoro-N,N-bis[(4-methoxyphenyl)methyl]-5-methyl-3-(4,4,5,5-tetramethyl-1,3,2-dioxaborolan-2-yl)aniline (647.2 mg, 1.32 mmol), tetrakis(triphenylphosphine)palladium (152.2 mg, 0.13 mmol) and cesium carbonate (643.7 mg, 1.98 mmol) in 1,4-dioxane (5 mL) and water (0.5 mL) was stirred at 100° C. for 1 hour. After completion, the resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1/1) to afford 3-((R)-1-(5-(3-(bis(4-methoxybenzyl)amino)-2-fluoro-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine(583 mg, 0.50 mmol, 75.3% yield) as a yellow solid. LC-MS: (ESI, m/Z): 1088.5 [M+H]⁺.

### Step 6: 3-((1R)-1-(5-(3-(bis(4-methoxybenzyl)amino)-2-fluoro-6-iodo-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine

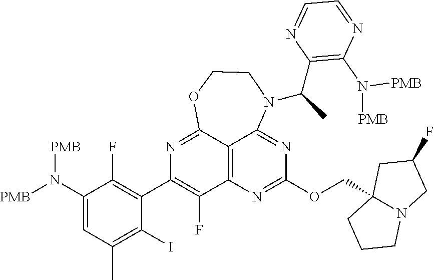

To a solution of 3-((R)-1-(5-(3-(bis(4-methoxybenzyl)amino)-2-fluoro-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine (680.0 mg, 0.62 mmol) in acetic acid (4 mL) and acetonitrile (4 mL) was added N-iodosuccinimide (168.7 mg, 0.75 mmol), the mixture was stirred at 25° C. for 0.5 hour. After completion, the reaction was quenched with saturated sodium thiosulfate solution. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1:1) to afford 3-((1R)-1-(5-(3-(bis(4-methoxybenzyl)amino)-2-fluoro-6-iodo-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine (480.0 mg, 0.37 mmol, 60% yield) as a yellow solid. LC-MS: (ESI, m/Z): 1214.4 [M+H]⁺.

### Step 7: 3-((1R)-1-(5-(3-(bis(4-methoxybenzyl)amino)-2-fluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine

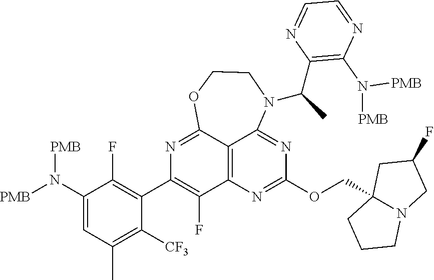

Under nitrogen, to a solution of 3-((1R)-1-(5-(3-(bis(4-methoxybenzyl)amino)-2-fluoro-6-iodo-5-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine (450.0 mg, 0.37 mmol) and copper (235.5 mg, 3.71 mmol) in N,N-dimethylformamide (4.5 mL) was added bis[(2,2-difluoro-2-fluorosulfonyl-acetyl)oxy]copper (1545.5 mg, 3.71 mmol) and stirred at −78° C. for 10 minutes, then the mixture was removed to 80° C. and stirred for 1 hour. After completion, the resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1:1) to afford 3-((1R)-1-(5-(3-(bis(4-methoxybenzyl)amino)-2-fluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine (200.0 mg, 0.14 mmol, 38.5% yield) as a yellow solid. LC-MS: (ESI, m/Z): 1156.5 [M+H]⁺.

### Step 8: 3-((1R)-1-(5-(3-amino-2-fluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine

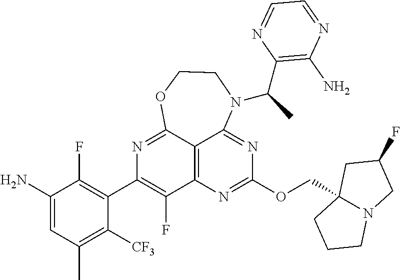 [SMILES: Cc1cc(N)c(F)c(-c2nc3c4c(nc(OC[C@@]56CCCN5C[C@H](F)C6)nc4c2F)N([C@H](C)c2nccnc2N)CCO3)c1C(F)(F)F]

A solution of 3-((1R)-1-(5-(3-(bis(4-methoxybenzyl)amino)-2-fluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine (180.0 mg, 0.16 mmol) in trifluoromethanesulfonic acid (0.5 mL) and 2,2,2-trifluoroacetic acid (5 mL) was stirred at 25° C. for 1 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (8:1) to afford crude product. The crude product was purified by Prep-HPLC with the following conditions: XBridge Shield RP18 OBD Column, 30*150 mm, 5 μm; Mobile Phase A: Water(10 mmol/L NH₄HCO₃), Mobile Phase B: acetonitrile; Flow rate: 60 mL/min; Gradient: 37% B to 61% B in 8 min, 61% B; Wave Length: 254/220 nm; RT1(min): 8 to afford 3-((1R)-1-(5-(3-amino-2-fluoro-5-methyl-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine (15.9 mg, 0.03 mmol, 15.1% yield). LC-MS: (ESI, m/Z): 675.6 [M+H]⁺.

Example 23: ¹H NMR (300 MHz, Methanol-d₄) δ7.98-7.89 (m, 1H), 7.86-7.85 (m, 1H), 6.87 (d, J=8.8 Hz, 1H), 6.67-6.48 (m, 1H), 5.33 (d, J=52.5 Hz, 1H), 4.70-4.54 (m, 1H), 4.51-4.39 (m, 1H), 4.42-4.20 (m, 2H), 4.09-3.80 (m, 2H), 3.38-3.37 (m, 1H), 3.30-3.17 (m, 2H), 3.13-2.97 (m, 1H), 2.41 (d, J=2.6 Hz, 3H), 2.37 -2.13 (m, 3H), 2.04-1.99 (m, 3H), 1.69 (dd, J=7.0, 1.6 Hz, 3H). LC-MS: (ESI, m/Z): 676.4 [M+H]⁺.

### Example 24: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)benzo[d]thiazol-2-amine

### Synthetic Route

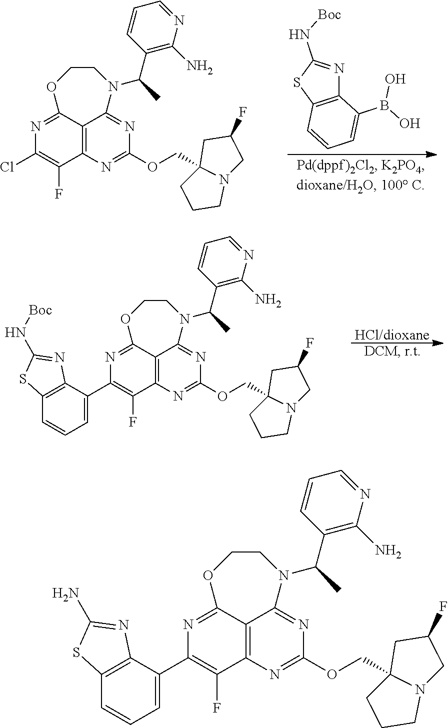

### Step 1n tert-butyl (43(10-((R)(1((2aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)benzo[d]thiazo-2-yl)carbamate

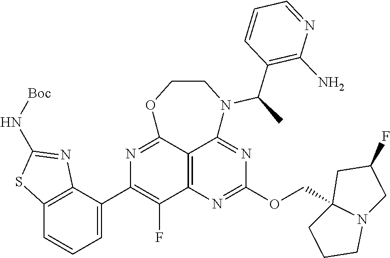 [SMILES: C[C@H](c1cccnc1N)N1CCOc2nc(-c3cccc4sc(NC(=O)OC(C)(C)C)nc34)c(F)c3nc(OC[C@@]45CCCN4C[C@H](F)C5)nc1c23]

Under nitrogen, a mixture of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphtha len-10-yl)ethyl)pyridin-2-yl)carbamate (150.0 mg, 0.24 mmol), (2-((tert-butoxycarbonyl)amino)benzo[d]thiazol-4-yl)boronic acid (107.1 mg, 0.36 mmol), [1,1′-bis(diphenylphosphino)ferrocene]dichloropalladium(II) (18.0 mg, 0.02 mmol) and potassium phosphate (154.5 mg, 0.73 mmol) in 1,4-dioxane (2 mL) and water (0.20 mL) was stirred at 100° C. for 2 hours. After completion, the reaction was diluted with water and extracted with ethyl acetate. The organic layers were combined, washed with brine, dried over anhydrous sodium sulfate and concentrated. The residue was purified by flash chromatography on silica gel eluting with dichloromethane/methanol (96:4) to afford tert-butyl (4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)benzo[d]thiazol-2-yl)carbamate (93 mg, 0.13 mmol, 52.4% yield) as a yellow solid. LCMS (ESI, m/Z): 732.3 [M+H]⁺.

### Step 2: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)benzo[d]thiazol-2-amine

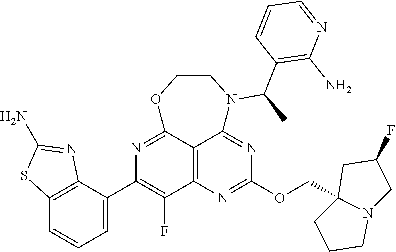 [SMILES: C[C@H](c1cccnc1N)N1CCOc2nc(-c3cccc4sc(N)nc34)c(F)c3nc(OC[C@@]45CCCN4C[C@H](F)C5)nc1c23]

A mixture of tert-butyl (4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)benzo[d]thiazol-2-yl)carbamate (93.0 mg, 0.13 mmol) and 4 M hydrochloric acid in dioxane (1 mL) in dichloromethane (1 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (84:16) to afford crude product. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9 to afford 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)benzo[d]thiazol-2-amine (17 mg, 0.03 mmol, 21.2% yield). LCMS (ESI, m/Z): 632.2 [M+H]⁺.

Example 24: ¹H NMR (300 MHz, Chloroform-d) 68.07 (dd, J=4.9, 1.7 Hz, 1H), 7.64 (d, J=7.8 Hz, 1H), 7.52 (dd, J=10.2, 7.4 Hz, 2H), 7.19 (t, J=7.8 Hz, 1H), 6.73-6.64 (m, 2H), 6.03 (s, 2H), 5.41-5.20 (m, 3H), 4.39 (dd, J=12.8, 6.0 Hz, 1H), 4.31 (s, 2H), 4.22 (dd, J=12.7, 6.6 Hz, 1H), 3.61-3.43 (m, 2H), 3.30-3.19 (m, 3H), 3.05-2.99 (m, 1H), 2.32-2.16 (m, 3H), 2.01-1.91 (m, 3H), 1.55 (d, J=6.8 Hz, 3H).

### Example 25: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-fluoronaphthalen-2-ol

### Synthetic Route

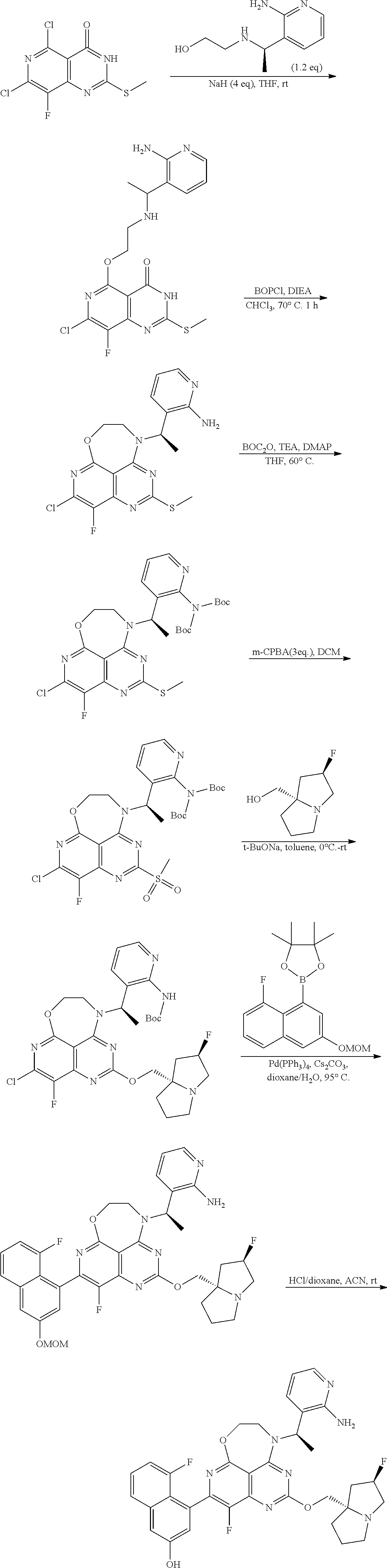

### Step 1: (R)-5-(2-((1-(2-aminopyridin-3-yl)ethyl)amino)ethoxy)-7-chloro-8-fluoro-2-(methylthio)pyrido[4,3-d]pyrimidin-4(3H)-one

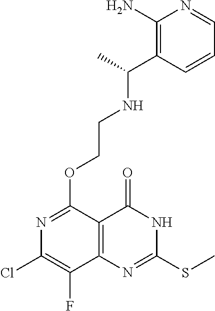 [SMILES: CSc1nc2c(F)c(Cl)nc(OCCN[C@H](C)c3cccnc3N)c2c(=O)[nH]1]

To a mixture of 2-[[(1R)-1-(2-amino-3-pyridyl)ethyl]amino]ethanol (750.0 mg, 4.14 mmol) in tetrahydrofuran (20 mL) was added sodium hydride (700.0 mg, 17.5 mmol, 60% dispersion in mineral oil), the mixture was stirred for 10 minutes at 0° C. Then 5,7-dichloro-8-fluoro-2-methylsulfanyl-3H-pyrido[4,3-d]pyrimidin-4-one (1000.0 mg, 3.57 mmol) was added, stirred for 1 h at room temperature. After completion, the reaction was quenched with saturated ammonium chloride solution. The resulting solution was diluted with water and extracted with ethyl acetate. The organic layers were combined, washed with brine, dried over anhydrous sodium sulfate and concentrated. The crude product would be directly used in the next step without purification. LCMS (ESI, m/Z): 425.1 [M+H]⁺.

### Step 2: (R)-3-(1-(5-chloro-4-fluoro-2-(methylthio)-8,9-dihydro-10H-7-oxa-1,3,6,10-tetraazacyclohepta[de]naphthalen-10-yl)ethyl)pyridin-2-amine

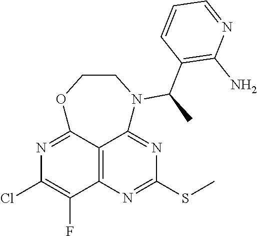 [SMILES: CSc1nc2c3c(nc(Cl)c(F)c3n1)OCCN2[C@H](C)c1cccnc1N]

A mixture of (R)-5-(2-((1-(2-aminopyridin-3-yl)ethyl)amino)ethoxy)-7-chloro-8-fluoro-2-(methylthio)pyrido[4,3-d]pyrimidin-4(3H)-one (2.00 g, crude), bis(2-oxo-3-oxazolidinyl)phosphinicchloride (1.50 g, 5.89 mmol) and N,N-diisopropylethylamine (4.04 mL, 23.21 mmol) in chloroform (40 mL) was stirred at 70° C. for 1 hour. The resulting solution was diluted with water and extracted with ethyl acetate. The organic layers were combined, washed with brine, dried over anhydrous sodium sulfate and concentrated. The crude product would be directly used in the next step without purification. LCMS (ESI, m/Z): 407.1 [M+H]⁺.

### Step 3: tert-butyl N-tert-butoxycarbonyl-N-[3-[(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl]-2-pyridyl]carbamate

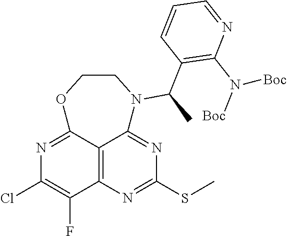 [SMILES: CSc1nc2c3c(nc(Cl)c(F)c3n1)OCCN2[C@H](C)c1cccnc1N(C(=O)OC(C)(C)C)C(=O)OC(C)(C)C]

A solution of (R)-3-(1-(5-chloro-4-fluoro-2-(methylthio)-8,9-dihydro-10H-7-oxa-1,3,6,10-tetraazacyclohepta[de]naphthalen-10-yl)ethyl)pyridin-2-amine (2.00 g, crude), di-tert-butyl dicarbonate (3.20 g, 14.66 mmol), triethylamine (6.89 mL, 49.41 mmol) and 4-dimethylaminopyridine (120.0 mg, 0.98 mmol) in tetrahydrofuran (40 mL) was stirred at 60° C. for 2 hours. After completion, the reaction was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (30:70) to afford tert-butyl N-tert-butoxycarbonyl-N-[3-[(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl]-2-pyridyl]carbamate (860 mg, 1.42 mmol, 28.8% yield) as a yellow solid. LCMS (ESI, m/Z): 607.2 [M+H]⁺.

### Step 4: tert-butyl N-tert-butoxycarbonyl-N-[3-[(1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl]-2-pyridyl]carbamate

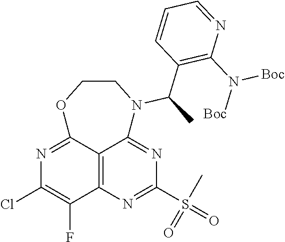 [SMILES: C[C@H](c1cccnc1N(C(=O)OC(C)(C)C)C(=O)OC(C)(C)C)N1CCOc2nc(Cl)c(F)c3nc(S(C)(=O)=O)nc1c23]

A mixture of tert-butyl N-tert-butoxycarbonyl-N-[3-[(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl]-2-pyridyl]carbamate (400.0 mg, 0.66 mmol) and 3-chloroperoxybenzoicacid (340.0 mg, 1.97 mmol) in dichloromethane (10 mL) was stirred at room temperature for 1 hour. The reaction was quenched with saturated sodium sulfite solution. The resulting solution was extracted with ethyl acetate. The organic layers were combined, washed with brine, dried over anhydrous sodium sulfate and concentrated. The residue was purified by flash chromatography on silica gel eluting with petroleum ether/ethyl acetate (30:70) to afford tert-butyl N-tert-butoxycarbonyl-N-[3-[(1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl]-2-pyridyl]carbamate (240 mg, 0.38 mmol, 57% yield) as a yellow solid. LCMS (ESI, m/Z):639.2 [M+H]⁺.

### Step 5: tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate

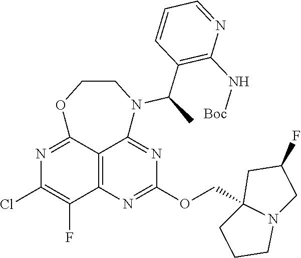 [SMILES: C[C@H](c1cccnc1NC(=O)OC(C)(C)C)N1CCOc2nc(Cl)c(F)c3nc(OC[C@@]45CCCN4C[C@H](F)C5)nc1c23]

Under nitrogen, to a mixture of ((2R,7aS)-2-fluorotetrahydro-1H-pyrrolizin-7a(5H)-yl)methanol (145.0 mg, 0.91 mmol) in toluene (7 mL) was added sodium tert-butoxide (130.8 mg, 1.36 mmol), the mixture was stirred for 10 minutes at 0° C., then tert-butyl N-tert-butoxycarbonyl-N-[3-[(1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl]-2-pyridyl]carbamate (290.0 mg, 0.45 mmol) was added, stirred for 1 hour at room temperature. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (96:4) to afford tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (180 mg, 0.29 mmol, 64.2% yield) as a yellow solid. LCMS (ESI, m/Z):618.2 [M+H]⁺.

### Step 6: 3-((R)-1-(4-fluoro-5-(8-fluoro-3-(methoxymethoxy)naphthalen-1-yl)-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

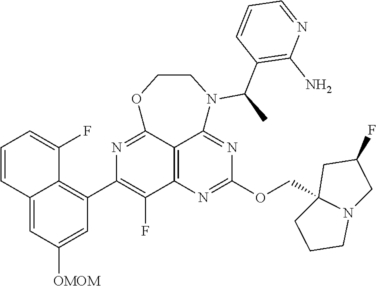

Under nitrogen, a mixture of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (170.0 mg, 0.28 mmol), 2-[8-fluoro-3-(methoxymethoxy)-1-naphthyl]-4,4,5,5-tetramethyl-1,3,2-dioxaborolane (140.0 mg, 0.42 mmol), tetrakis(triphenylphosphine)palladium(0) (32.0 mg, 0.03 mmol) and cesium carbonate (180.0 mg, 0.55 mmol) in 1,4-dioxane (4 mL) and Water (0.8 mL) was stirred at 95° C. for 1 hour. After completion, the resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (10:90) to afford 3-((R)-1-(4-fluoro-5-(8-fluoro-3-(methoxymethoxy)naphthalen-1-yl)-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (140 mg, 0.18 mmol, 64.6% yield) as a yellow solid. LCMS (ESI, m/Z):688.3 [M+H]⁺.

### Step 7: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-fluoronaphthalen-2-ol

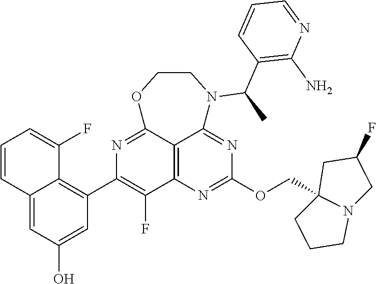 [SMILES: C[C@H](c1cccnc1N)N1CCOc2nc(-c3cc(O)cc4cccc(F)c34)c(F)c3nc(OC[C@@]45CCCN4C[C@H](F)C5)nc1c23]

A mixture of 3-((R)-1-(4-fluoro-5-(8-fluoro-3-(methoxymethoxy)naphtha len-1-yl)-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphtha len-10-yl)ethyl)pyridin-2-amine (140.0 mg, 0.16 mmol) and 4 M hydrochloric acid in dioxane (1 mL) in acetonitrile (3 mL) was stirred at room temperature for 1 hour. After completion, the solvent was removed under vacuum. The crude product was purified by Prep-HPLC with the following conditions: Column: XSelect CSH Fluoro Phenyl, 30*150 mm, 5 μm; Mobile Phase A: Water (10 mmol/L NH₄HCO₃), Mobile Phase B: ACN; Flow rate: 60 mL/min; Gradient: 38% B to 68% B in 7 min; Wave Length: 254 nm; RT1(min): 6.5 to afford 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphtha len-5-yl)-5-fluoronaphthalen-2-ol (38.2 mg, 0.06 mmol, 37.6% yield) as a white solid. LCMS (ESI, m/Z):644.2 [M+H]⁺.

Example 25: ¹H NMR (300 MHz, DMSO-d₆, ppm) δ 10.22 (s, 1H), 7.98 (dd, J=4.9, 1.6 Hz, 1H), 7.73-7.57 (m, 2H), 7.50-7.38 (m, 1H), 7.34 (t, J=2.3 Hz, 1H), 7.12 (d, J=2.4 Hz, 1H), 7.10-6.95 (m, 1H), 6.68 (dd, J=7.5, 4.9 Hz, 1H), 6.39 (q, J=7.0, 6.5 Hz, 1H), 5.74 (s, 2H), 5.29 (d, J=54.1Hz, 1H), 4.58-4.21 (m, 2H), 4.14 (s, 2H), 3.75 (dd, J=16.0, 6.3 Hz, 1H), 3.47-3.42 (m, 1H), 3.15-2.95 (m, 3H), 2.91-2.76 (m, 1H), 2.29-2.13 (m, 1H), 2.11-1.99 (m, 2H), 1.92-1.72 (m, 3H), 1.58 (d, J=5.7 Hz, 3H).

### Example 26: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)naphthalen-2-ol

### Synthetic Route

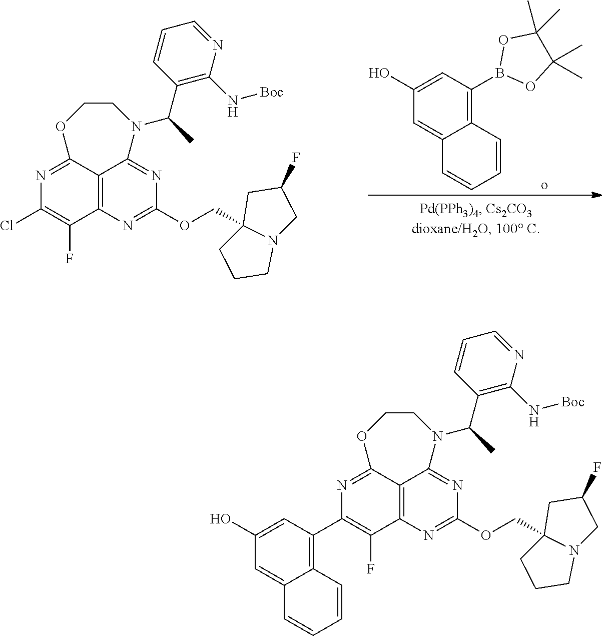 [SMILES: C[C@H](c1cccnc1NC(=O)OC(C)(C)C)N1CCOc2nc(Cl)c(F)c3nc(OC[C@@]45CCCN4C[C@H](F)C5)nc1c23 ; CC1(C)OB(c2cc(O)cc3ccccc23)OC1(C)C ; C[C@H](c1cccnc1NC(=O)OC(C)(C)C)N1CCOc2nc(-c3cc(O)cc4ccccc34)c(F)c3nc(OC[C@@]45CCCN4C[C@H](F)C5)nc1c23]

### Step 1: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)naphthalen-2-ol

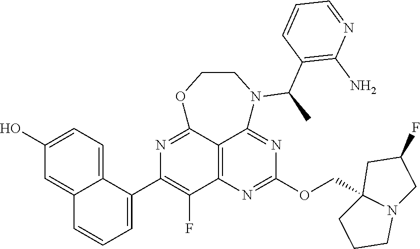 [SMILES: C[C@H](c1cccnc1N)N1CCOc2nc(-c3cccc4cc(O)ccc34)c(F)c3nc(OC[C@@]45CCCN4C[C@H](F)C5)nc1c23]

Under nitrogen, a solution of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (250.0 mg, 0.35 mmol), 4-(4,4,5,5-tetramethyl-1,3,2-dioxaborolan-2-yl)naphthalen-2-ol (113.0 mg, 0.42 mmol), tetrakis(triphenylphosphine)palladium (81.0 mg, 0.07 mmol) and cesium carbonate (341.0 mg, 1.05 mmol) in 1,4-dioxane (3 mL) and water (0.6 mL) was stirred at 100° C. for 1 hour. After completion, the resulting solution was diluted with water and extracted with ethyl acetate. Then the organic layers were combined, washed with brine, dried over anhydrous sodium sulfate and concentrated under vacuum. The crude product was purified by Prep-HPLC with the following conditions:Column: XBridge Prep OBD C18 Column, 30*150 mm, 5 μm; Mobile Phase A: Water(10 mmol/L NH₄HCO₃), Mobile Phase B: ACN(0.1% DEA)—HPLC—merk; Flow rate: 60 mL/min; Gradient: 29% B to 50% B in 9 min, 50% B; Wave Length: 254/220 nm; RT1(min): 8.8 to afford 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)naphthalen-2-ol (45.1 mg, 0.07 mmol, 20.6% yield). LC-MS: (ESI, m/Z): 626.4 [M+H]⁺

Example 26: ¹H NMR (300 MHz, DMSO-d₆, ppm) δ 9.96 (s, 1H), 7.98 (dd, J=4.8, 1.6 Hz, 1H), 7.78 (d, J=8.2 Hz, 1H), 7.61 (dd, J=16.6, 7.9 Hz, 2H), 7.43 (t, J=7.5 Hz, 1H), 7.30-7.21 (m, 2H), 7.18 (d, J=2.4 Hz, 1H), 6.70-6.66 (m, 1H), 6.38 (q, J=7.6, 6.9 Hz, 1H), 5.74 (s, 2H), 5.29 (d, J=54.3 Hz, 1H), 4.46 (dd, J=12.6, 6.5 Hz, 1H), 4.32 (dd, J=12.8, 6.4 Hz, 1H), 4.15 (s, 2H), 3.76 (dd, J=15.9, 6.4 Hz, 1H), 3.41 (dd, J=16.0, 6.7 Hz, 1H), 3.11-3.00 (m, 3H), 2.91-2.76 (m, 1H), 2.22-1.94 (m, 3H), 1.88-1.77 (m, 3H), 1.58 (d, J=6.8 Hz, 3H).

### Example 27: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-ethylnaphthalen-2-ol

### Synthetic Route

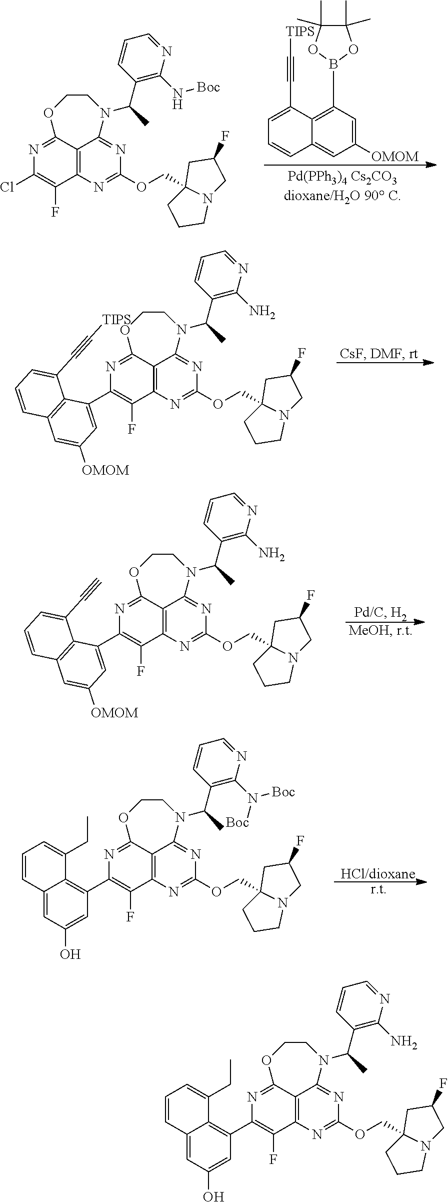

### Step 1: 3-((R)-1-(4-fluoro-2-(((2R,7aS)-2-fluorotetrahydro-1H-pyrrolizin-7a(5H)-yl)methoxy)-5-(3-(methoxymethoxy)-8-((triisopropylsilyl)ethynyl)naphthalen-1-yl)-8,9-dihydro-10H-7-oxa-1,3,6,10-tetraazacyclohepta[de]naphthalen-10-yl)ethyl)pyridin-2-amine

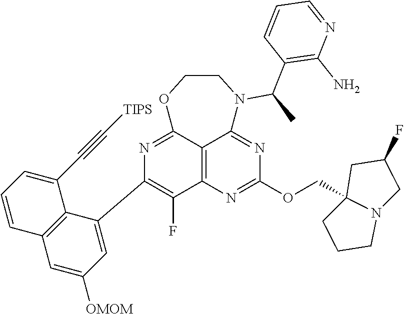

Under nitrogen, a solution of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (600.0 mg, 0.97 mmol), triisopropyl((6-(methoxymethoxy)-8-(4,4,5,5-tetramethyl-1,3,2-dioxaborolan-2-yl)naphthalen-1-yl)ethynyl)silane (496.0 mg, 1.00 mmol), tetrakis(triphenylphosphine)palladium (194.0 mg, 0.17 mmol) and cesium carbonate (818.0 mg, 2.51 mmol) in water (1.6 mL) and 1,4-dioxane (8 mL) was stirred for 1 hour at 90° C. After completion, the resulting solution was diluted with water and extracted with ethyl acetate. The organic layers were combined, washed with brine, dried over anhydrous sodium sulfate and concentrated under vacuum. The residue was purified by flash chromatography on silica gel eluting with dichloromethane/methanol (47/3) to afford 3-((R)-1-(4-fluoro-2-(((2R,7aS)-2-fluorotetrahydro-1H-pyrrolizin-7a(5H)-yl)methoxy)-5-(3-(methoxymethoxy)-8-((triisopropylsilyl)ethynyl)naphthalen-1-yl)-8,9-dihydro-10H-7-oxa-1,3,6,10-tetraazacyclohepta[de]naphthalen-10-yl)ethyl)pyridin-2-amine (430 mg, 0.50 mmol, 52% yield) as a yellow solid. LC-MS: (ESI, m/Z): 850.4[M+H]⁺

### Step 2: 3-((R)-1-(5-(8-ethynyl-3-(methoxymethoxy)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

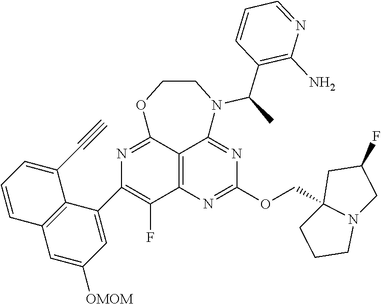

A solution of 3-((R)-1-(4-fluoro-2-(((2R,7aS)-2-fluorotetrahydro-1H-pyrrolizin-7a(5H)-yl)methoxy)-5-(3-(methoxymethoxy)-8-((triisopropylsilyl)ethynyl)naphthalen-1-yl)-8,9-dihydro-10H-7-oxa-1,3,6,10-tetraazacyclohepta[de]naphthalen-10-yl)ethyl)pyridin-2-amine (410.0 mg, 0.48 mmol) and caesium fluoride (415.0 mg, 2.73 mmol) in N,N-dimethylformamide (10 mL) was stirred at room temperature for 1 hour.

After completion, the resulting solution was diluted with water and extracted with ethyl acetate. The organic layers were combined, washed with brine, dried over anhydrous sodium sulfate and concentrated under vacuum. The residue was purified by flash chromatography on silica gel eluting with dichloromethane/methanol (47/3) to afford 3-((R)-1-(5-(8-ethynyl-3-(methoxymethoxy)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (290 mg, 0.41 mmol, 85.4% yield) as a yellow solid. LC-MS: (ESI, m/Z): 694.3[M+H]⁺

### Step 3: 3-((R)-1-(5-(8-ethyl-3-(methoxymethoxy)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

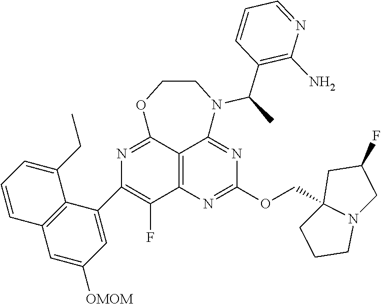

Under hydrogen, a solution of 3-((R)-1-(5-(8-ethynyl-3-(methoxymethoxy)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (250.0 mg, 0.36 mmol) and Pd/C (59.5 mg, 0.06 mmol) in methyl alcohol (5 mL) was stirred at room temperature for 1 hour. After completion, the solids were filtered out. The filtrate was concentrated under reduced pressure to afford 3-((R)-1-(5-(8-ethyl-3-(methoxymethoxy)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (180 mg, 0.26 mmol, 71.6% yield) as a yellow solid. LC-MS: (ESI, m/Z):698.3 [M+H]⁺

### Step 4: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-ethylnaphthalen-2-ol

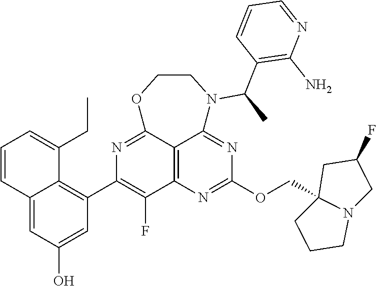 [SMILES: CCc1cccc2cc(O)cc(-c3nc4c5c(nc(OC[C@@]67CCCN6C[C@H](F)C7)nc5c3F)N([C@H](C)c3cccnc3N)CCO4)c12]

To solution of 3-((R)-1-(5-(8-ethyl-3-(methoxymethoxy)naphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (170.0 mg, 0.24 mmol) in acetonitrile (2 mL) was added 4 M hydrochloric acid in dioxane (2 mL), the mixture was stirred at room temperature for 0.5 hour. After completion, the solvent was removed under vacuum. The crude product was purified by Prep-HPLC with the following conditions: Column: Xselect CSH C18 OBD Column 30*150 mm 5 μm; Mobile Phase A: Water(0.1% FA), Mobile Phase B: ACN; Flow rate: 60 mL/min; Gradient: 10% B to 22% B in 9 min, 22% B; Wave Length: 254/220 nm; RT1(min): 9 to afford 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-ethylnaphthalen-2-ol (41.1 mg, 0.06 mmol, 30.9% yield). LC-MS: (ESI, m/Z):654.4 [M+H]⁺

Example 27: ¹H NMR (300 MHz, DMSO-d₆, ppm) δ 9.80 (s, 1H), 7.92-7.82 (m, 1H), 7.61-7.45 (m, 2H), 7.33-7.17 (m, 1H), 7.14 (d, J=2.7 Hz, 1H), 7.01 (dd, J=7.3, 3.4 Hz, 1H), 6.82 (d, J=2.6 Hz, 1H), 6.63-6.49 (m, 1H), 6.26 (q, J=7.0, 6.5 Hz, 1H), 5.63 (d, J=22.4 Hz, 2H), 5.18 (d, J=54.2 Hz, 1H), 4.41-4.26 (m, 1H), 4.26-4.11 (m, 1H), 4.03 (s, 2H), 3.73-3.51 (m, 1H), 3.41-3.20 (m, 1H), 3.08-2.86 (m, 3H), 2.79-2.63 (m, 1H), 2.23 (q, J=7.1Hz, 2H), 2.13-1.89 (m, 3H), 1.76-1.63 (m, 3H), 1.47 (t, J=7.4 Hz, 3H), 0.92-0.85 (m, 3H).

### Examples 28A, 28B, 28C, & 28D: 3-((R)-1-((R)-5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)pyridin-2-amine

### 3-((S)-1-((R)-5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)pyridin-2-amine

### 3-((R)-1-((S)-5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)pyridin-2-amine

### 3-((S)-1-((S)-5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)pyridin-2-amine

### Synthetic Route

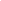

### Step 1: 1-((1-(2-(bis(4-methoxybenzyl)amino)pyridin-3-yl)ethyl)amino)propan-2-ol

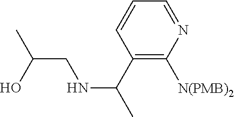

A solution of 1-[2-[bis[(4-methoxyphenyl)methyl]amino]-3-pyridyl]ethanone (5.00 g, 13.28 mmol) and 1-amino-2-propanol (2.99 g, 39.85 mmol) in methyl alcohol (50 mL) was added titanium tetraisopropanolate (11.32 g, 39.85 mmol) and stirred at 80° C. for 24 hours. Then the reaction solution was cooled to room temperature. Sodium borohydride (2.52 g, 66.41 mmol) was slowly added into the reaction solution at 25° C. and stirred at 80° C. for 1.5 hours. After completion, the reaction was quenched with saturated ammonium chloride solution. Then reaction mixture was concentrated under reduced pressure to remove the methyl alcohol, diluted with water and extracted with ethyl acetate, dried over anhydrous sodium sulfate and concentrated under vacuum. The residue was purified by flash chromatography on silica gel eluting with methanol/dichloromethane (1:8) to afford the title compound (3.20 g, 7.35 mmol, 55.3% yield) as a white solid. LC-MS: (ESI, m/Z): 436.3 [M+H]⁺

### Step 2: 5-((1-((1-(2-(bis(4-methoxybenzyl)amino)pyridin-3-yl)ethyl)amino)propan-2-yl)oxy)-7-chloro-8-fluoro-2-(methylthio)pyrido[4,3-d]pyrimidin-4(3H)-one

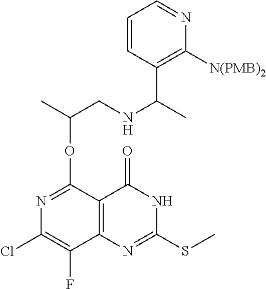

Under nitrogen, a solution of 1-[1-[2-[bis[(4-methoxyphenyl)methyl]amino]-3-pyridyl]ethylamino]propan-2-ol (2.33 g, 5.36 mmol) in tetrahydrofuran (10 mL) was slowly added sodium hydride (0.57 g, 14.28 mmol, 60% purity) at 0° C. for 10 min. Then the reaction mixture was added into a solution of 5,7-dichloro-8-fluoro-2-methylsulfanyl-3H-pyrido[4,3-d]pyrimidin-4-one (1.00 g, 3.57 mmol) in tetrahydrofuran (10 mL) and stirred at 25° C. for 4 hours. After completion, the reaction was quenched with saturated ammonium chloride solution, diluted with water, extracted with ethyl acetate, dried over anhydrous sodium sulfate and concentrated under vacuum. The residue was purified by flash chromatography on silica gel eluting with ethyl acetate/petroleum ether (1:8) to afford the title compound (1.70 g, 2.50 mmol, 70.1% yield) as a white solid. LC-MS: (ESI, m/Z): 679.2 [M+H]⁺

### Step 3: 3-(1-(5-chloro-4-fluoro-8-methyl-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine

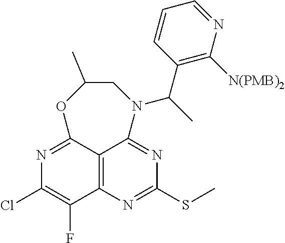

A solution of 5-[2-[1-[2-[bis[(4-methoxyphenyl)methyl]amino]-3-pyridyl]ethylamino]-1-methyl-ethoxy]-7-chloro-8-fluoro-2-methylsulfanyl-3H-pyrido[4,3-d]pyrimidin-4-one (1.00 g, 1.47 mmol), bis(2-oxo-3-oxazolidinyl)phosphinic chloride (0.56 g, 2.21 mmol) and N,N-diisopropylethylamine (0.57 g, 4.42 mmol) in chloroform (10 mL) was stirred at 70° C. for 1 hour. After completion, the reaction mixture was diluted with ethyl acetate, washed with water, dried over anhydrous sodium sulfate and concentrated under vacuum. The residue was purified by flash chromatography on silica gel eluting with ethyl acetate/petroleum ether (1:5) to afford the title compound (700.0 mg, 1.06 mmol, 71.9% yield) as a white solid. LC-MS: (ESI, m/Z): 661.2 [M+H]⁺

### Step 4: 3-(1-(5-chloro-4-fluoro-8-methyl-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine

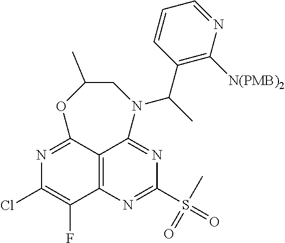

A solution of 3-[1-(7-chloro-6-fluoro-11-methyl-3-methylsulfanyl-10-oxa-2,4,8,13-tetrazatricyclo[7.4.1.05, 14]tetradeca-1,3,5(14),6,8-pentaen-13-yl)ethyl]-N,N-bis[(4-methoxyphenyl)methyl]pyridin-2-amine (1.90 g, 2.87 mmol) in N,N-dimethylformamide (20 mL) was slowly added 3-chloroperoxybenzoic acid (1.98 g, 11.49 mmol) and stirred at 25° C. for 1 hour. After completion, the reaction was quenched with saturated sodium sulfite solution, diluted with water, extracted with ethyl acetate, dried over anhydrous sodium sulfate and concentrated under vacuum. The residue was purified by flash chromatography on silica gel eluting with ethyl acetate/petroleum ether (1:5) to afford the title compound (1.50 g, 2.16 mmol, 75.3% yield) as a yellow solid. LC-MS: (ESI, m/Z): 693.2 [M+H]⁺

### Step 5: 3-(1-(5-chloro-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)-N,N-bis(4-methoxybenzyl)pyridin-2-amine

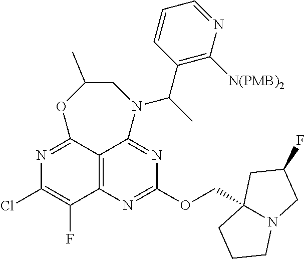

A solution of ((2R,7aS)-2-fluorotetrahydro-1H-pyrrolizin-7a(5H)-yl)methanol (0.83 g, 5.19 mmol) in toluene (8 mL) was slowly added sodium tert-butoxide (0.83 g, 8.66 mmol) and stirred at 0° C. for 15 minutes. Then the reaction mixture was added into a solution of 3-[1-(7-chloro-6-fluoro-11-methyl-3-methylsulfonyl-10-oxa-2,4,8,13-tetrazatricyclo[7.4.1.05, 14]tetradeca-1,3,5(14),6,8-pentaen-13-yl)ethyl]-N,N-bis[(4-methoxyphenyl)methyl]pyridin-2-amine (1.20 g, 1.73 mmol) in toluene (8 mL) and stirred at 25° C. for 1 hour. After completion, the reaction mixture was quenched with saturated ammonium chloride solution, diluted with water, extracted with ethyl acetate, dried over anhydrous sodium sulfate and concentrated under vacuum. The residue was purified by flash chromatography on silica gel eluting with ethyl acetate/petroleum ether (1:5) to afford the title compound (635.0 mg, 0.82 mmol, 47.5% yield) as a yellow solid. LC-MS: (ESI, m/Z): 772.3 [M+H]⁺

### Step 6: 3-(1-(5-(5-(bis(4-methoxybenzyl)amino)-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)-N,N-bis(4-methoxybenzyl)pyridin-2-amine

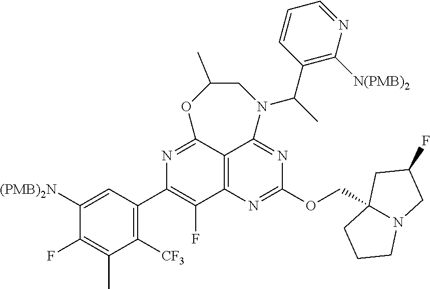

Under nitrogen, a solution of 3-(1-(5-chloro-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)-N,N-bis(4-methoxybenzyl)pyridin-2-amine (400.0 mg, 0.52 mmol), [5-[bis[(4-methoxyphenyl)methyl]amino]-4-fluoro-3-methyl-2-(trifluoromethyl)phenyl]boronic acid (296.6 mg, 0.62 mmol), tetrakis(triphenylphosphine)palladium (59.8 mg, 0.05 mmol) and cesium carbonate (506.2 mg, 1.55 mmol) in 1,4-dioxane (4 mL) and water (0.6 mL) was stirred at 100° C. for 2 hours. After completion, the reaction mixture was diluted with ethyl acetate, washed with water, dried over anhydrous sodium sulfate and concentrated under vacuum. The residue was purified by flash chromatography on silica gel eluting with methanol/dichloromethane (1:8) to afford the title compound (400.0 mg, 0.34 mmol, 66% yield) as a white solid. LC-MS: (ESI, m/Z): 1169.5 [M+H]⁺

### Step 7: 3-((R)-1-((R)-5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)pyridin-2-amine & 3-((S)-1-((R)-5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)pyridin-2-amine & 3-((R)-1-((S)-5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)pyridin-2-amine & 3-((S)-1-((S)-5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)pyridin-2-amine

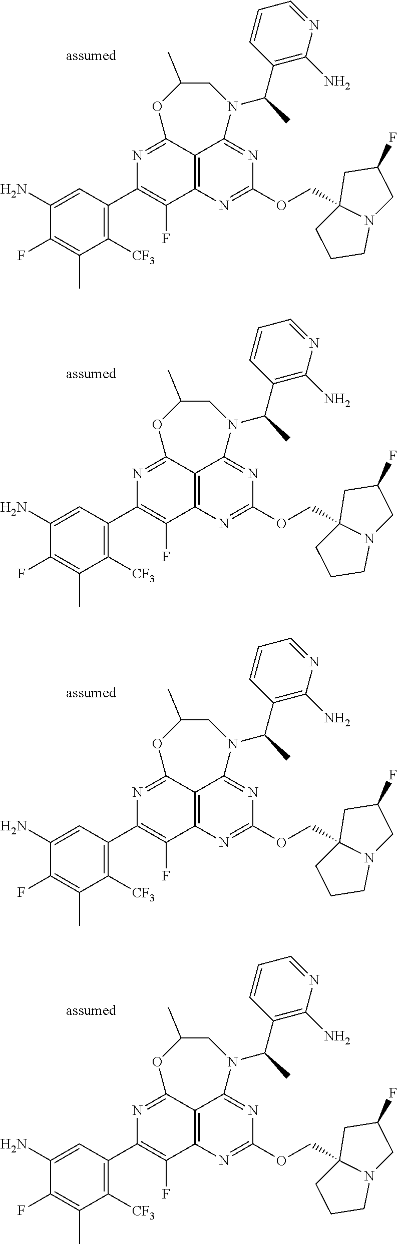 [SMILES: Cc1c(F)c(N)cc(-c2nc3c4c(nc(OC[C@@]56CCCN5C[C@H](F)C6)nc4c2F)N([C@H](C)c2cccnc2N)CC(C)O3)c1C(F)(F)F ; Cc1c(F)c(N)cc(-c2nc3c4c(nc(OC[C@@]56CCCN5C[C@H](F)C6)nc4c2F)N([C@H](C)c2cccnc2N)CC(C)O3)c1C(F)(F)F ; Cc1c(F)c(N)cc(-c2nc3c4c(nc(OC[C@@]56CCCN5C[C@H](F)C6)nc4c2F)N([C@H](C)c2cccnc2N)CC(C)O3)c1C(F)(F)F ; Cc1c(F)c(N)cc(-c2nc3c4c(nc(OC[C@@]56CCCN5C[C@H](F)C6)nc4c2F)N([C@H](C)c2cccnc2N)CC(C)O3)c1C(F)(F)F]

A solution of 3-(1-(5-(5-(bis(4-methoxybenzyl)amino)-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)-N,N-bis(4-methoxybenzyl)pyridin-2-amine (700.0 mg, 0.60 mmol) in trifluoroacetic acid and trifluoromethanesulfonic acid (7 mL) was stirred at 25° C. for 30 minutes. After completion, the reaction solution was concentrated under reduced pressure, diluted with dichloromethane and adjusted to pH=7 with saturated sodium bicarbonate solution. The resulting solution was extracted with dichloromethane, dried over anhydrous sodium sulfate and concentrated under vacuum. The residue was purified by flash chromatography on silica gel eluting with dichloromethane/methanol (10:1) to afford the product. The product was further purified by Prep-HPLC with the following conditions (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm) to afford 160 mg product. The product was separated by Chiral-Prep-HPLC with the following conditions: Column: XBridge Prep OBD C18 Column, 30*150 mm, 5 μm; Mobile Phase A: Water(10 mmol/L NH4HCO3), Mobile Phase B: ACN; Flow rate: 60 mL/min; Gradient: 17% B to 42% B in 9 min; Wave Length: 254/220 nm; RT1(min): 8.9 to afford the first compound 3-((R)-1-((R)-5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)pyridin-2-amine (40.6 mg, 0.059 mmol, 9.7% yield) as a white solid, the second compound 3-((S)-1-((R)-5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)pyridin-2-amine (5.0 mg, 0.0073 mmol, 1.2% yield), the third compound 3-((R)-1-((S)-5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)pyridin-2-amine (2.2 mg, 0.0032 mmol, 0.5% yield) as a white solid and the fourth compound 3-((S)-1-((S)-5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8-methyl-8,9-dihydro-10H-7-oxa-1,3,6,10-tetraazacyclohepta[de]naphthalen-10-yl)ethyl)pyridin-2-amine (64.4 mg, 0.094 mmol, 15.6% yield) as a white solid.

Example 28a: ¹H NMR (300 MHz, DMSO-d₆, ppm) δ 8.00 (dd, J=4.9, 1.6 Hz, 1H), 7.80-7.58 (m, 1H), 6.70 (dd, J=7.5, 4.9 Hz, 1H), 6.64-6.42 (m, 1H), 6.42-6.24 (m, 1H), 6.01 (s, 2H), 5.71 (s, 2H), 5.29 (d, J=54.4 Hz, 1H), 4.13 (s, 3H), 3.68-3.46 (m, 2H), 3.21-2.95 (m, 3H), 2.91-2.73 (m, 1H), 2.32 (s, 3H), 2.26-1.96 (m, 3H), 1.92-1.70 (m, 3H), 1.52 (d, J=6.8 Hz, 3H), 1.09 (d, J=6.5 Hz, 3H). LC-MS: (ESI, m/Z): 689.3 [M+H]⁺. Column: CHIRALPAK IA-3, 4.6*50 mm, 3 um; detected at 254 nm; Mobile Phase: (Hex:DCM=3:1)(0.1% DEA):IPA=85:15 Flow: 1 mL/min; Retention time: 1.492 min (first peak)

Example 28b: ¹H NMR (300 MHz, Methanol-d₄, ppm) 68.06-7.92 (m, 1H), 7.86-7.71 (m, 1H), 6.91-6.72 (m, 1H), 6.68-6.45 (m, 2H), 5.36 (d, J=53.9 Hz, 1H), 4.63 (s, 1H), 4.38 (s, 2H), 4.23 (s, 1H), 3.83-3.33 (m, 4H), 3.18-2.94 (m, 1H), 2.56-2.33 (m, 4H), 2.33-2.13 (m, 2H), 2.13-1.83 (m, 3H), 1.64 (dd, J=16.7, 6.9 Hz, 3H), 1.18 (d, J=6.6 Hz, 3H). LC-MS: (ESI, m/Z): 689.3 [M+H]⁺. Column: CHIRALPAK IA-3, 4.6*50 mm, 3 um; detected at 254 nm; Mobile Phase: (Hex:DCM=3:1)(0.1% DEA):IPA=85:15; Flow: 1 mL/min; Retention time: 2.236 min (second peak)

Example 28c: ¹H NMR (300 MHz, Methanol-d₄, ppm) δ 7.95 (dd, J=5.2, 1.6 Hz, 1H), 7.83-7.70 (m, 1H), 6.77 (dd, J=7.5, 5.1Hz, 1H), 6.69-6.48 (m, 2H), 5.33 (d, J=53.7 Hz, 1H), 4.64 (s, 1H), 4.34 (q, J=10.5 Hz, 2H), 3.76-3.60 (m, 1H), 3.45-3.33 (m, 2H), 3.30-3.13 (m, 2H), 3.15-2.92 (m, 1H), 2.38 (s, 3H), 2.33-2.08 (m, 3H), 2.08-1.80 (m, 3H), 1.67 (d, J=6.9 Hz, 3H), 1.29 (s, 3H). LC-MS: (ESI, m/Z): 689.3 [M+H]⁺. Column: CHIRAL Cellulose-SB, 4.6*100 mm, 3 μm; detected at 254 nm; Mobile Phase: Hex(0.1% DEA):IPA=50:50; Flow: 1.0 mL/min; Retention time: 2.756 min (third peak)

Example 28d: ¹H NMR (300 MHz, DMSO-d₆, ppm) δ 7.99 (dd, J=4.9, 1.7 Hz, 1H), 7.66 (dd, J=7.6, 1.8 Hz, 1H), 6.69 (dd, J=7.5, 4.9 Hz, 1H), 6.64-6.43 (m, 1H), 6.34 (q, J=6.8 Hz, 1H), 6.00 (s, 2H), 5.70 (s, 2H), 5.29 (d, J=54.4 Hz, 1H), 4.25-3.99 (m, 3H), 3.20-2.95 (m, 5H), 2.93-2.76 (m, 1H), 2.31 (s, 3H), 2.24-2.10 (m, 1H), 2.10-1.92 (m, 2H), 1.90-1.69 (m, 3H), 1.51 (d, J=6.8 Hz, 3H), 1.08 (d, J=6.5 Hz, 3H). LC-MS: (ESI, m/Z): 689.3 [M+H]⁺. Column: CHIRALPAK IE-3 4.6*50 mm, 3 μm; detected at 254 nm; Mobile Phase: MtBE(0.1% DEA):(MeOH:DCM=1:1)=95:5; Flow: 1.0 mL/min; Retention time: 4.868 min (fourth peak)

### Example 29: 3-((R)-1-(5-(3-Amino-8-ethynyinaphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine

### Synthetic Route

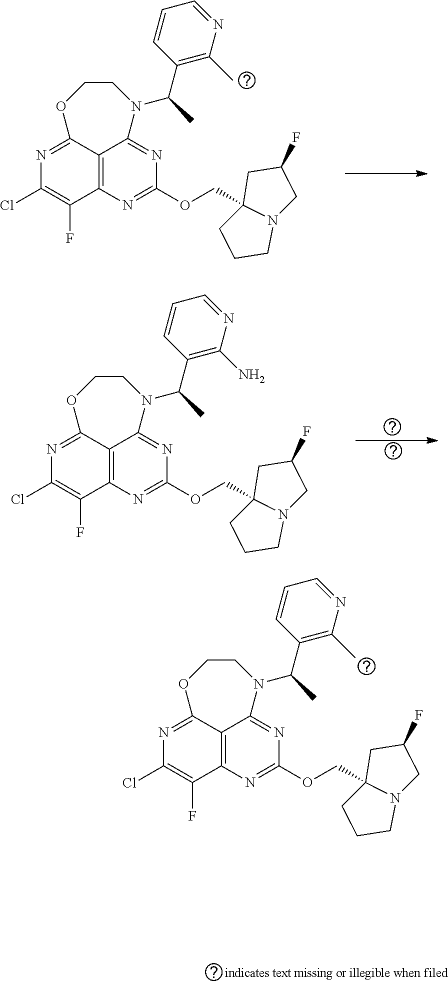

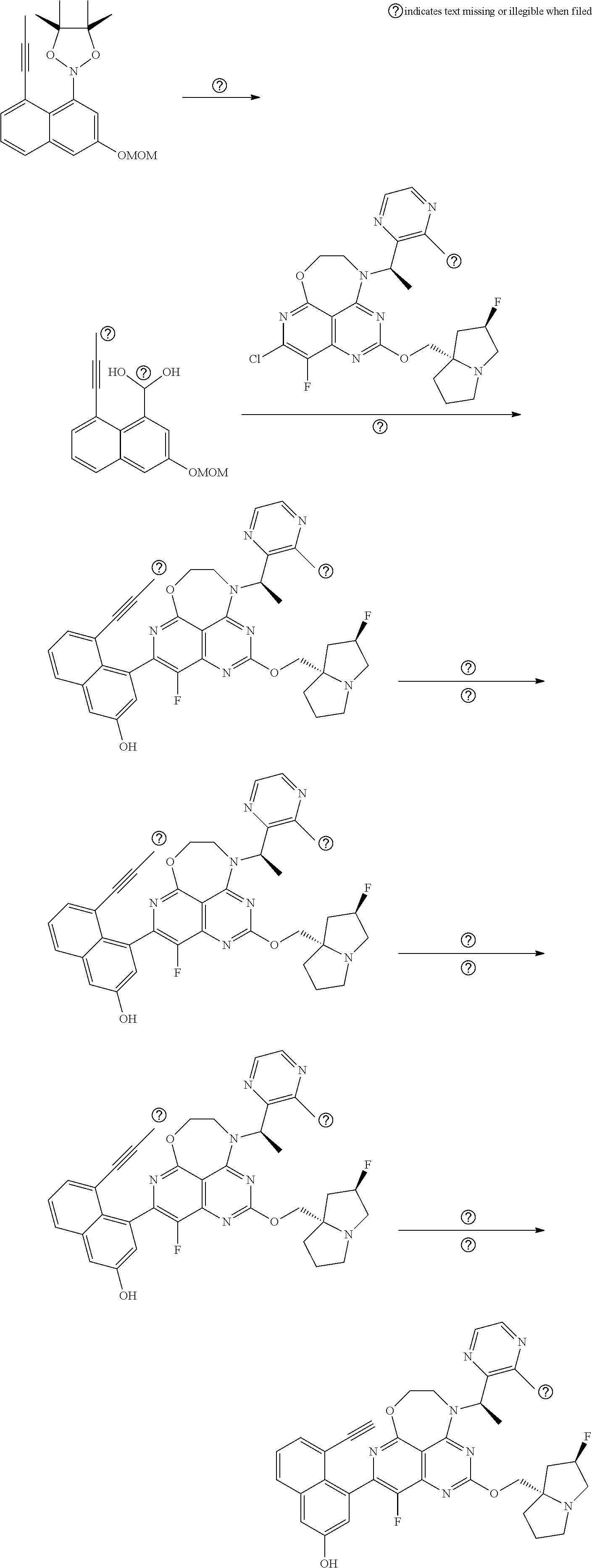

### Step 1: 3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-22amine

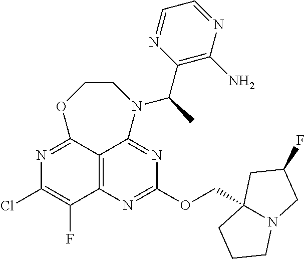 [SMILES: C[C@H](c1nccnc1N)N1CCOc2nc(Cl)c(F)c3nc(OC[C@@]45CCCN4C[C@H](F)C5)nc1c23]

A solution of 3-((R)-1-(5-chloro-4-fluoro-2-(((2R,7aS)-2-fluorotetrahydro-1H-pyrrolizin-7a(5H)-yl)methoxy)-8,9-dihydro-1 OH-7-oxa-1,3,6,10-tetraazacyclohepta[de]naphthalen-1 O-yl)ethyl)-N,N-bis(4-methoxybenzyl)pyrazin-2-amine (1.1 g, 1.4 mmol) in trifluoroacetic acid (10.0 mL) and trifluoromethanesulfonic acid (1.0 mL) was stirred at room temperature for 0.5 hour. After completion, the reaction mixture was diluted with dichloromethane and adjusted to pH=7 with saturated sodium bicarbonate solution. The resulting solution was extracted with dichloromethane and the organic layers were combined. The organic layers were dried over anhydrous sodium sulfate and concentrated under vacuum. The residue was purified by flash chromatography on silica gel eluting with methanol/dichloromethane (1:8) to afford the titled compound (600.0 mg, 1.2 mmol, 82% yield) as a white solid. LC-MS: (ESI, m/Z): 519.1 [M+H]⁺

### Step 2: tert-butyl N-(3-((1R)-1-(3-(((2R,7aS)-2-fluoro-hexahydro-1H-pyrrolizin-7a-yl)methoxy)-7-chloro-6-fluoro-10-oxa-2,4,8,13-tetraazatricyclo(7.4.1.0^{5, 14})tetradeca-1,3,5(14),6,8-pentaen-13-yl)ethyl)pyrazin-2-yl)-N-((tert-butoxy)carbonyl)carbamate

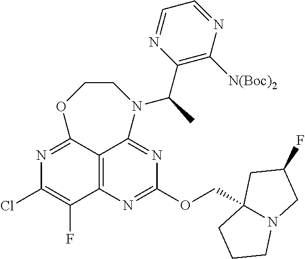

A solution of 3-((R)-1-(5-Chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine (1.10 g, 2.10 mmol), di-tert-butyl dicarbonate (2.80 g, 12.7 mmol), triethylamine (1.30 g, 12.7 mmol) and 4-dimethylaminopyridine (52.0 mg, 0.4 mmol) in tetrahydrofuran (10 mL) was stirred at 70° C. for 1 hour. After completion, the reaction mixture was diluted with ethyl acetate, washed with water, dried over anhydrous sodium sulfate and concentrated under vacuum. The residue was purified by flash chromatography on silica gel eluting with methanol/dichloromethane (1:8) to afford the titled compound (1.20 g, 1.70 mmol, 79% yield) as a white solid. LC-MS: (ESI, m/Z): 719.4 [M+H]⁺

### Step 3: (3-hydroxy-8-((triisopropylsilyl)ethynyl)naphthalen-1-yl)boronic acid

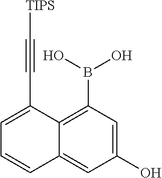

A solution of triisopropyl-[2-[6-(methoxymethoxy)-8-(4,4,5,5-tetramethyl-1,3,2-dioxaborolan-2-yl)-1-naphthyl]ethynyl]silane (700.0 mg, 1.42 mmol) in hydrochloric acid in 1,4-dioxane (5 mL, 4 M) was stirred at 25° C. for 1 hour. After completion, the reaction mixture was concentrated under vacuum. The residue was purified by flash chromatography on silica gel eluting with petroleum ether/ethyl acetate (9:1) to afford the title compound (575.0 mg, 1.40 mmol, 99.3% yield) as a white solid. LC-MS: (ESI, m/Z): 369.3 [M+H]⁺

### Step 4: tert-butyl N-(3-((1R)-1-(3-(((2R,7aS)-2-fluoro-hexahydro-1H-pyrrolizin-7a-yl)methoxy)-6-fluoro-7-(3-hydroxy-8-(2-(tris(propan-2-yl)silyl)ethynyl)naphthalen-1-yl)-10-oxa-2,4,8,13-tetraazatricyclo(7.4.1.0^{5, 14})tetradeca-1,3,5(14),6,8-pentaen-13-yl)ethyl)pyrazin-2-yl)-N-((tert-butoxy)carbonyl)carbamate

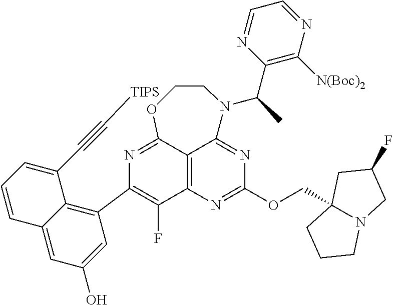

Under nitrogen, tert-butyl N-(3-((1R)-1-(3-(((2R,7aS)-2-fluoro-hexahydro-1H-pyrrolizin-7a-yl)methoxy)-7-chloro-6-fluoro-10-oxa-2,4,8,13-tetraazatricyclo(7.4.1.0^{5, 14})tetradeca-1,3,5(14),6,8-pentaen-13-yl)ethyl)pyrazin-2-yl)-N-((tert-butoxy)carbonyl)carbamate (1.00 g, 1.4 mmol), [3-hydroxy-8-(2-triisopropylsilylethynyl)-1-naphthyl]boronic acid (768.0 mg, 2.1 mmol), [(di(1-adamantyl)-butylphosphine)-2-(2′-amino-1,1′-biphenyl)]palladium(II) methanesulfonate (101.0 mg, 0.14 mmol) and potassium phosphate tribasic (884.0 mg, 4.2 mmol) in tetrahydrofuran/water (5:1) (10.0 mL) was stirred at 80° C. for 1.5 hours. After completion, the reaction mixture was diluted with water, extracted with ethyl acetate and the organic layers were combined. The organic layer was dried over anhydrous sodium sulfate and concentrated under vacuum. The residue was purified by flash chromatography on silica gel eluting with ethyl acetate/petroleum ether (1:8) to afford the titled compound (1.0 g, 1.0 mmol, 71% yield) as a white solid. LC-MS: (ESI, m/Z): 1007.2 [M+H]⁺

### Step 5: tert-butyl N-(3-((1R)-1-(3-(((2R,7aS)-2-fluoro-hexahydro-1H-pyrrolizin-7a-yl)methoxy)-6-fluoro-7-(3-(trifluoromethanesulfonyloxy)-8-(2-(tris(propan-2-yl)silyl)ethynyl)naphthalen-1-yl)-10-oxa-2,4,8,13-tetraazatricyclo(7.4.1.0^{5, 14})tetradeca-1,3,5(14),6,8-pentaen-13-yl)ethyl)pyrazin-2-yl)-N-((tert-butoxy)carbonyl)carbamate

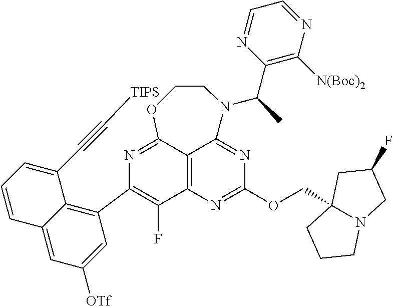

A solution of tert-butyl N-(3-((1R)-1-(3-(((2R,7aS)-2-fluoro-hexahydro-1H-pyrrolizin-7a-yl)methoxy)-6-fluoro-7-(3-hydroxy-8-(2-(tris(propan-2-yl)silyl)ethynyl)naphthalen-1-yl)-10-oxa-2,4,8,13-tetraazatricyclo(7.4.1.0^{5, 14})tetradeca-1,3,5(14),6,8-pentaen-13-yl)ethyl)pyrazin-2-yl)-N-((tert-butoxy)carbonyl)carbamate(300.0 mg, 0.3 mmol), 1,1,1-trifluoro-N-phenyl-N-((trifluoromethyl)sulfonyl)methanesulfonamide (212.0 mg, 0.6 mmol) and N,N-diisopropylethylamine (154.0 mg, 1.2 mmol) in dichloromethane (3.0 mL) was stirred at room temperature for 1.5 hours. After completion, the reaction mixture was diluted with water, extracted with ethyl acetate and the organic layers were combined. The organic layer was dried over anhydrous sodium sulfate and concentrated under vacuum. The residue was purified by flash chromatography on silica gel eluting with ethyl acetate/petroleum ether (1:8) to afford the titled compound (300.0 mg, 0.26 mmol, 88% yield) as a white solid. LC-MS: (ESI, m/Z): 1139.5 [M+H]⁺

### Step 6: tert-butyl N-(3-((1R)-1-(3-(((2R,7aS)-2-fluoro-hexahydro-1H-pyrrolizin-7a-yl)methoxy)-7-(3-(((tert-butoxy)carbonyl)amino)-8-(2-(tris(propan-2-yl)silyl)ethynyl)naphthalen-1-yl)-6-fluoro-10-oxa-2,4,8,13-tetraazatricyclo(7.4.1.0^{5, 14})tetradeca-1,3,5(14),6,8-pentaen-13-yl)ethyl)pyrazin-2-yl)-N-((tert-butoxy)carbonyl)carbamate

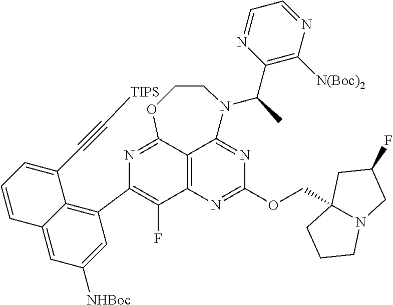

Under nitrogen atmosphere, a solution of tert-butyl N-(3-((1R)-1-(3-(((2R,7aS)-2-fluoro-hexahydro-1H-pyrrolizin-7a-yl)methoxy)-6-fluoro-7-(3-(trifluoromethanesulfonyloxy)-8-(2-(tris(propan-2-yl)silyl)ethynyl)naphthalen-1-yl)-10-oxa-2,4,8,13-tetraazatricyclo(7.4.1.0^{5, 14})tetradeca-1,3,5(14),6,8-pentaen-13-yl)ethyl)pyrazin-2-yl)-N-((tert-butoxy)carbonyl)carbamate (280.0 mg, 0.25 mmol), tert-butyl carbamate (58.0 mg, 0.49 mmol), tris(dibenzylideneacetone)dipalladium (22 mg, 0.02 mmol), 4,5-bis(diphenylphosphino)-9,9-dimethylxanthene (28.0 mg, 0.05 mmol) and cesium carbonate (240.0 mg, 0.74 mmol) in 1,4-dioxane (3.0 mL) was stirred at 100° C. for 1.5 hours. After completion, the reaction mixture was diluted with water, extracted with ethyl acetate and the organic layers were combined. The organic layer was dried over anhydrous sodium sulfate and concentrated under vacuum. The residue was purified by flash chromatography on silica gel eluting with ethyl acetate/petroleum ether (1:8) to afford the titled compound (150.0 mg, 0.14 mmol, 55% yield) as a white solid. LC-MS: (ESI, m/Z): 1006.3 [M+H]⁺

### Step 7: tert-butyl N-(3-((1R)-1-(3-(((2R,7aS)-2-fluoro-hexahydro-1H-pyrrolizin-7a-yl)methoxy)-7-(3-(((tert-butoxy)carbonyl)amino)-8-ethynylnaphthalen-1-yl)-6-fluoro-10-oxa-2,4,8,13-tetraazatricyclo(7.4.1.0^{5, 14})tetradeca-1,3,5(14),6,8-pentaen-13-yl)ethyl)pyrazin-2-yl)-N-((tert-butoxy)carbonyl)carbamate

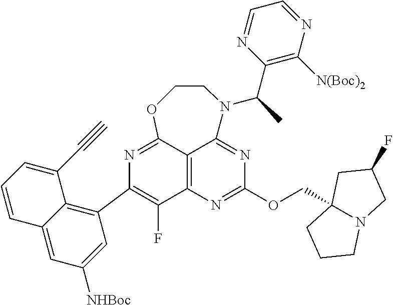

A solution of tert-butyl N-(3-((1R)-1-(3-(((2R,7aS)-2-fluoro-hexahydro-1H-pyrrolizin-7a-yl)methoxy)-7-(3-(((tert-butoxy)carbonyl)amino)-8-(2-(tris(propan-2-yl)silyl)ethynyl)naphthalen-1-yl)-6-fluoro-10-oxa-2,4,8,13-tetraazatricyclo(7.4.1.0^{5, 14})tetradeca-1,3,5(14),6,8-pentaen-13-yl)ethyl)pyrazin-2-yl)-N-((tert-butoxy)carbonyl)carbamate (140.0 mg, 0.14 mmol) and caesium fluoride (196 mg, 1.26 mmol) in N,N-dimethylacetamide (5.0 mL) was stirred at room temperature for 2.0 hours. After completion, the reaction mixture was diluted with water and extracted with dichloromethane. The combined organic layers were washed with brine, dried over anhydrous sodium sulfate and concentrated in vacuum. The residue was purified by flash chromatography on silica gel eluting with dichloromethane/methanol (95:5) to afford titled compound (100.0 mg, 0.10 mmol, 75% yield) as a light yellow oil. LC-MS: (ESI, m/Z): 950.6 [M+H]⁺

### Step 8: 3-((R)-1-(5-(3-amino-8-ethynylnaphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine

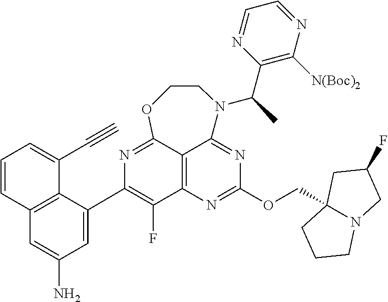

A solution of tert-butyl N-(3-((1R)-1-(3-(((2R,7aS)-2-fluoro-hexahydro-1H-pyrrolizin-7a-yl)methoxy)-7-(3-(((tert-butoxy)carbonyl)amino)-8-ethynylnaphthalen-1-yl)-6-fluoro-10-oxa-2,4,8,13-tetraazatricyclo(7.4.1.0^{5, 14})tetradeca-1,3,5(14),6,8-pentaen-13-yl)ethyl)pyrazin-2-yl)-N-((tert-butoxy)carbonyl)carbamate (100.0 mg, 0.11 mmol) in hydrochloric acid/1,4-dioxane (5.0 mL, 2M in 1,4-dioxane) was stirred at room temperature for 4 hours. After completion, the mixture was concentrated under reduced pressure. Then diluted with dichloromethane and adjusted to pH=7 with saturated sodium bicarbonate solution. The resulting solution was extracted with dichloromethane and the organic layers were combined. The organic layer was dried over anhydrous sodium sulfate and concentrated under vacuum. The residue was purified by flash chromatography on silica gel eluting with dichloromethane/methanol (10:1) to afford the crude product. Then the crude product was purified by Prep-HPLC with the following conditions (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm) to afford the titled compound (12.4 mg, 0.02 mmol, 18% yield) as a yellow solid. LC-MS: (ESI, m/Z): 650.4 [M+H]⁺

Example 29: ¹H NMR (300 MHz, DMSO-d₆, ppm) δ 7.96 (dd, J=2.7, 0.9 Hz, 1H), 7.80 (dd, J=2.7, 1.8 Hz, 1H), 7.67 (dd, J=6.8, 2.9 Hz, 1H), 7.34-7.22 (m, 2H), 7.03-6.90 (m, 2H), 6.57-6.28 (m, 3H), 5.65 (s, 2H), 5.29 (d, J=54.4 Hz, 1H), 4.64-4.25 (m, 2H), 4.16-4.01 (m, 2H), 3.99-3.58 (m, 3H), 3.16-2.95 (m, 3H), 2.90-2.75 (m, 1H), 2.18-1.95 (m, 3H), 1.89-1.72 (m, 3H), 1.60 (dd, J=6.9, 3.2 Hz, 3H).

### Example 30: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-ethynylquinolin-2-amine

### Synthetic Route

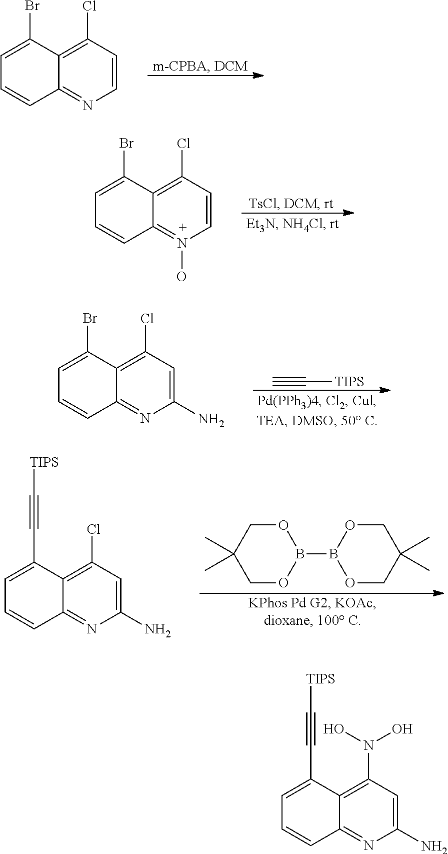

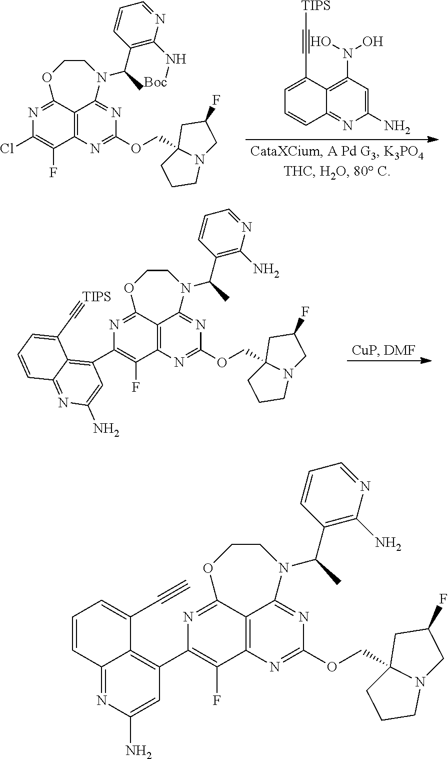

### Step 1: 5-bromo-4-chloroquinoline 1-oxide

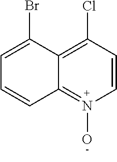

To a mixture of 5-bromo-4-chloro-quinoline (2.00 g, 8.25 mmol) in dichloromethane (20 mL) was added 3-chloroperoxybenzoicacid (3.56 g, 20.62 mmol), the mixture was stirred for 1 hour at room temperature. After completion, the reaction was quenched with saturated sodium thiosulfate solution. The resulting solution was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (1:1) to afford 5-bromo-4-chloroquinoline 1-oxide (1.20 g, 4.64 mmol, 56.3% yield) as a yellow solid. LC-MS: (ESI, m/Z): 257.9 [M+H]⁺

### Step 2: 5-bromo-4-chloroquinolin-2-amine

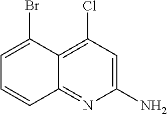 [SMILES: Nc1cc(Cl)c2c(Br)cccc2n1]

Under nitrogen, to a mixture of 5-bromo-4-chloroquinoline 1-oxide (1.20 g, 4.64 mmol) in dichloromethane (15 mL) was added triethylamine (4.70 g, 46.42 mmol) and p-toluenesulfonylchloride (2.65 g, 13.93 mmol), the mixture was stirred for 15 minutes at room temperature. Then ammoniumchloride (2.48 g, 46.42 mmol) was added, stirred for 2 hours at room temperature.

After completion, the reaction was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (1:1) to afford 5-bromo-4-chloroquinolin-2-amine (1.06 g, 4.12 mmol, 88.7% yield) as a yellow solid. LC-MS: (ESI, m/Z): 256.9 [M+H]⁺

### Step 3: 4-chloro-5-((triisopropylsilyl)ethynyl)quinolin-2-amine

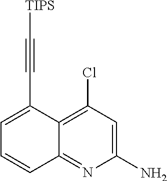

Under nitrogen, a mixture of 5-bromo-4-chloroquinolin-2-amine (1.06 g, 4.12 mmol), (triisopropylsilyl)acetylene (2.25 g, 12.35 mmol), bis(triphenylphosphine)palladium(ii)dichloride (579.5 mg, 0.82 mmol) and copper(i) iodide (156.8 mg, 0.82 mmol) in triethylamine (416.5 mg, 4.12 mmol) and dimethyl sulfoxide (5 mL) was stirred for 3 hours at 50° C. After completion, the reaction was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (1:1) to afford 4-chloro-5-((triisopropylsilyl)ethynyl)quinolin-2-amine (468 mg, 1.30 mmol, 31.7% yield) as a yellow solid. LC-MS: (ESI, m/Z): 359.2 [M+H]⁺

### Step 4: (2-amino-5-((triisopropylsilyl)ethynyl)quinolin-4-yl)boronic acid

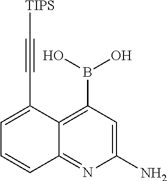

Under nitrogen, a mixture of 4-chloro-5-((triisopropylsilyl)ethynyl)quinolin-2-amine (100.0 mg, 0.28 mmol), bis(neopentylglycolato)diboron (629.2 mg, 2.79 mmol), XPhos Pd G2 (21.9 mg, 0.03 mmol) and potassium acetate (82.0 mg, 0.84 mmol) in 1,4-dioxane (2 mL) was stirred at 100° C. for 2 hours. After completion, the reaction was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (1:1) to afford (2-amino-5-((triisopropylsilyl)ethynyl)quinolin-4-yl)boronic acid (94.0 mg, 0.25 mmol, 91.6% yield) as a yellow oil. LC-MS: (ESI, m/Z): 369.2 [M+H]⁺

### Step 5: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-((triisopropylsilyl)ethynyl)quinolin-2-amine

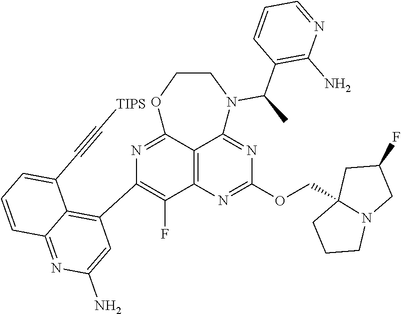

Under nitrogen, a mixture of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (100.0 mg, 0.16 mmol), (2-amino-5-((triisopropylsilyl)ethynyl)quinolin-4-yl)boronic acid (89.4 mg, 0.24 mmol), CataCxium A Pd G3 (11.7 mg, 0.02 mmol) and potassium phosphate (103 mg, 0.49 mmol) in tetrahydrofuran (2 mL) and water (0.4 mL) was stirred at 80° C. for 2 h. After completion, the reaction was diluted with water and extracted with ethyl acetate. The organic layers were combined, washed with brine, dried over anhydrous sodium sulfate and concentrated. The residue was purified by flash chromatography on silica gel eluting with dichloromethane/methanol (96:4) to afford 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-((triisopropylsilyl)ethynyl)quinolin-2-amine (34 mg, 0.04 mmol, 26.1% yield) as a yellow solid. LC-MS: (ESI, m/Z): 806.4 [M+H]⁺

### Step 6: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-ethynylquinolin-2-amine

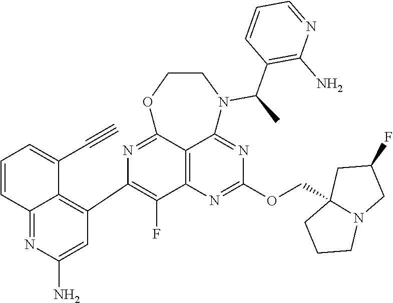 [SMILES: C#Cc1cccc2nc(N)cc(-c3nc4c5c(nc(OC[C@@]67CCCN6C[C@H](F)C7)nc5c3F)N([C@H](C)c3cccnc3N)CCO4)c12]

A mixture of 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-((triisopropylsilyl)ethynyl)quinolin-2-amine (34.0 mg, 0.04 mmol) and cesium fluoride (32.0 mg, 0.21 mmol) in N,N-dimethylformamide (2 mL) was stirred at room temperature for 2 h. After completion, the reaction was diluted with water and extracted with ethyl acetate. The organic layers were combined, washed with brine, dried over anhydrous sodium sulfate and concentrated. The crude product was purified by Prep-HPLC with the following conditions: Column: XSelect CSH Fluoro Phenyl, 30*150 mm, 5 μm; Mobile Phase A: Water(10 mmol/L NH₄HCO₃), Mobile Phase B: ACN; Flow rate: 60 mL/min; Gradient: 41% B to 64% B in 10 min, 64% B; Wave Length: 220/254 nm; RT1(min): 8.11 to afford 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-ethynylquinolin-2-amine (3.8 mg, 0.01 mmol, 13.9% yield) as a white solid. LC-MS: (ESI, m/Z): 650.2 [M+H]⁺

Example 30: ¹H NMR (300 MHz, Methanol-d₄, ppm) δ 7.96 (d, J=5.1Hz, 1H), 7.76 (d, J=8.0 Hz, 1H), 7.65 (dd, J=8.3, 1.2 Hz, 1H), 7.50 (ddd, J=8.5, 7.2, 1.7 Hz, 1H), 7.40 (ddd, J=7.3, 3.4, 1.4 Hz, 1H), 6.87 (d, J=2.2 Hz, 1H), 6.79 (ddd, J=6.9, 5.1, 1.6 Hz, 1H), 6.63-6.57 (m, 1H), 5.31 (d, J=53.7 Hz, 1H), 4.68-4.39 (m, 3H), 4.38-4.23 (m, 2H), 3.85-3.66 (m, 1H), 3.65-3.38 (m, 1H), 3.29-3.21 (m, 2H), 3.20-3.12 (m, 1H), 3.09-2.96 (m, 1H), 2.45-2.08 (m, 3H), 2.08-1.83 (m, 3H), 1.67 (d, J=6.9 Hz, 3H).

### Example 31: 3-((R)-1-(5-(5-amino-4-fluoro-3-methyl-2-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-7-oxa-1,3,6,10-tetraazacyclohepta[de]naphthalen-10-yl)ethyl)pyridin-2-amine

### Synthetic Route

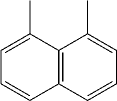

### Step 1:tert-butyl N-tert-butoxycarbonyl-N-(3-((1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl)-2-pyridyl)carbamate

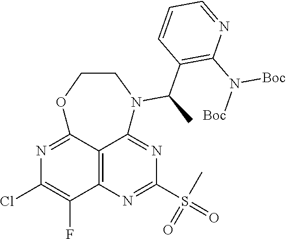 [SMILES: C[C@H](c1cccnc1N(C(=O)OC(C)(C)C)C(=O)OC(C)(C)C)N1CCOc2nc(Cl)c(F)c3nc(S(C)(=O)=O)nc1c23]

A solution of tert-butyl N-tert-butoxycarbonyl-N-(3-((1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl)-2-pyridyl)carbamate (1.00 g, 1.65 mmol) and 3-chloroperoxybenzoic acid (710.4 mg, 4.12 mmol) in dichloromethane (10 mL) was stirred at 25° C. for 0.5 hours. After completion, the resulting solution was quenched with saturated sodium bisulfite solution and extracted with ethyl acetate. Then the organic layers were combined, washed with brine, dried over anhydrous sodium sulfate and concentrated under vacuum. The residue was purified by flash chromatography on silica gel eluting with petroleum ether/ethyl acetate (3:7) to afford tert-butyl N-tert-butoxycarbonyl-N-(3-((1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl)-2-pyridyl)carbamate (710.0 mg, 1.11 mmol, 67.4% yield) as a yellow solid. LC-MS: (ESI, m/Z): 639.1 [M+H]⁺

### Step 2: tert-butyl N-(3-((1R)-1-(3-(((1R,7′aS)-3,3-difluoro-hexahydrospiro(cyclopropane-1,2′-pyrrolizine)-7′a-yl)methoxy)-7-chloro-6-fluoro-10-oxa-2,4,8,13-tetraazatricyclo(7.4.1.0^{5, 14})tetradeca-1,3,5(14),6,8-pentaen-13-yl)ethyl)pyridin-2-yl)-N-((tert-butoxy)carbonyl)carbamate

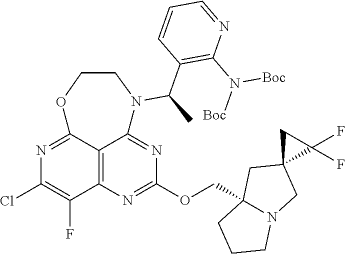 [SMILES: C[C@H](c1cccnc1N(C(=O)OC(C)(C)C)C(=O)OC(C)(C)C)N1CCOc2nc(Cl)c(F)c3nc(OC[C@@]45CCCN4C[C@]4(CC4(F)F)C5)nc1c23]

A solution of ((1R,7a'S)-2,2-difluorodihydro-1′H,3′H-spiro(cyclopropane-1,2′-pyrrolizin)-7a′(5′H)-yl)methanol (352.0 mg, 1.73 mmol) in tetrahydrofuran (6 mL) was added sodium hydride (139.0 mg, 3.47 mmol, 60% dispersion in mineral oil) at 0° C. The resulting solution was stirred for 10 minutes at 0° C. Then tert-butyl N-tert-butoxycarbonyl-N-(3-((1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl)-2-pyridyl)carbamate (555.0 mg, 0.87 mmol) was added and stirred at 0° C. for 0.5 hours. After completion, the reaction was quenched with saturated ammonium chloride solution. The solvent was concentrated under vacuum. The resulting solution was diluted with ethyl acetate and washed with water. The organic layer was washed with brine and dried over anhydrous sodium sulfate and concentrated under vacuum. The residue was purified by a reversed-phase chromatography directly with the following conditions: Column, C18 spherical 20-35 μm 100A; mobile phase, A: water, B: acetonitrile, B % (5%-95% in 30 min); to afford the title compound (420.0 mg, 0.55 mmol, 63.5% yield) as a yellow solid. LC-MS: (ESI, m/Z): 762.3 [M+H]⁺

### Step 3: tert-butyl N-(3-((1R)-1-(3-(((1R,7′aS)-3,3-difluoro-hexahydrospiro(cyclopropane-1,2′-pyrrolizine)-7′a-yl)methoxy)-7-(5-(bis((4-methoxyphenyl)methyl)amino)-4-fluoro-3-methyl-2-(trifluoromethyl)phenyl)-6-fluoro-10-oxa-2,4,8,13-tetraazatricyclo(7.4.1.0^{5, 14})tetradeca-1,3,5(14),6,8-pentaen-13-yl)ethyl)pyridin-2-yl)-N-((tert-butoxy)carbonyl)carbamate

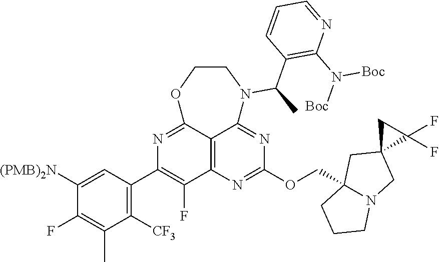

Under nitrogen, a solution of tert-butyl N-(3-((1R)-1-(3-(((1R,7′aS)-3,3-difluoro-hexahydrospiro(cyclopropane-1,2′-pyrrolizine)-7′a-yl)methoxy)-7-chloro-6-fluoro-10-oxa-2,4,8,13-tetraazatricyclo(7.4.1.0^{5, 14})tetradeca-1,3,5(14),6,8-pentaen-13-yl)ethyl)pyridin-2-yl)-N-((tert-butoxy)carbonyl)carbamate (372.0 mg, 0.49 mmol), (5-(bis(4-methoxybenzyl)amino)-4-fluoro-3-methyl-2-(trifluoromethyl)phenyl)boronic acid (280.0 mg, 0.59 mmol), tetrakis(triphenylphosphine)palladium (56.5 mg, 0.05 mmol) and cesium carbonate (478.0 mg, 1.47 mmol) in 1,4-dioxane (4 mL) and water (0.8 mL) was stirred for 1 hour at 100° C. After completion, the resulting solution was diluted with ethyl acetate and washed with water. The organic layer was washed with brine and dried over anhydrous sodium sulfate and concentrated under vacuum. The residue was purified by flash chromatography on silica gel eluting with dichloromethane/methanol (9:1) to afford the title compound (510.0 mg, 0.44 mmol, 90.1% yield) as a yellow solid. LC-MS: (ESI, m/Z): 1159.2 [M+H]⁺

### Step 4:3-((R)-1-(5-(5-amino-4-fluoro-3-methyl-2-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-7-oxa-1,3,6,10-tetraazacyclohepta[de]naphthalen-10-yl)ethyl)pyridin-2-amine

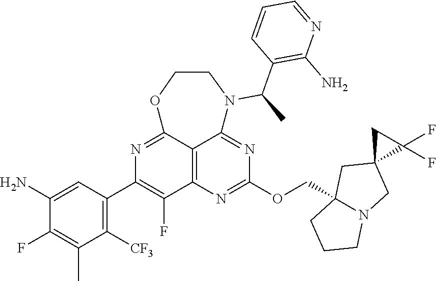 [SMILES: Cc1c(F)c(N)cc(-c2nc3c4c(nc(OC[C@@]56CCCN5C[C@]5(CC5(F)F)C6)nc4c2F)N([C@H](C)c2cccnc2N)CCO3)c1C(F)(F)F]

A solution of tert-butyl N-(3-((1R)-1-(3-(((1R,7′aS)-3,3-difluoro-hexahydrospiro(cyclopropane-1,2′-pyrrolizine)-7′a-yl)methoxy)-7-(5-(bis((4-methoxyphenyl)methyl)amino)-4-fluoro-3-methyl-2-(trifluoromethyl)phenyl)-6-fluoro-10-oxa-2,4,8,13-tetraazatricyclo(7.4.1.0^{5, 14})tetradeca-1,3,5(14),6,8-pentaen-13-yl)ethyl)pyridin-2-yl)-N-((tert-butoxy)carbonyl)carbamate

(484.0 mg, 0.42 mmol) in trifluoromethanesulfonic acid (0.4 mL) and trifluoroacetic acid (4 mL) was stirred at 25° C. for 1 hour. After completion, the mixture was concentrated under vacuum. The residue was adjusted to pH=7 by saturated sodium bicarbonate solution. The mixture was diluted with dichloromethane and washed with water. The organic layer was washed with brine and dried over anhydrous sodium sulfate and concentrated under vacuum. The crude product was purified by Prep-HPLC with the following conditions: Column: YMC-Actus Triart C18 ExRS, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9 to afford the title compound (95.3 mg, 0.13 mmol, 31.8% yield) as a white solid. LC-MS: (ESI, m/Z): 719.2 [M+H]⁺

Example 31: ¹H NMR (300 MHz, DMSO-d₆, ppm) δ 7.97 (dd, J=4.9, 1.6 Hz, 1H), 7.63 (dd, J=7.4, 1.7 Hz, 1H), 6.67 (dd, J=7.5, 4.9 Hz, 1H), 6.55 (d, J=8.7 Hz, 1H), 6.44-6.28 (m, 1H), 6.02 (s, 2H), 5.73 (s, 2H), 4.43 (dd, J=12.7, 6.3 Hz, 1H), 4.35-4.13 (m, 3H), 3.73 (dd, J=16.0, 6.4 Hz, 1H), 3.49-3.38 (m, 1H), 3.10 (dd, J=11.9, 6.8 Hz, 1H), 3.06-2.97 (m, 1H), 2.72 (d, J=11.8 Hz, 1H), 2.60-2.53 (m, 1H), 2.38-2.24 (m, 3H), 2.09 (dd, J=13.6, 5.7 Hz, 1H), 2.05-1.96 (m, 1H), 1.91 (d, J=13.4 Hz, 1H), 1.86-1.71 (m, 2H), 1.69-1.43 (m, 6H).

### Example 32: 3-((R)-1-(5-(5-amino-4-fluoro-3-methyl-2-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Synthetic Route

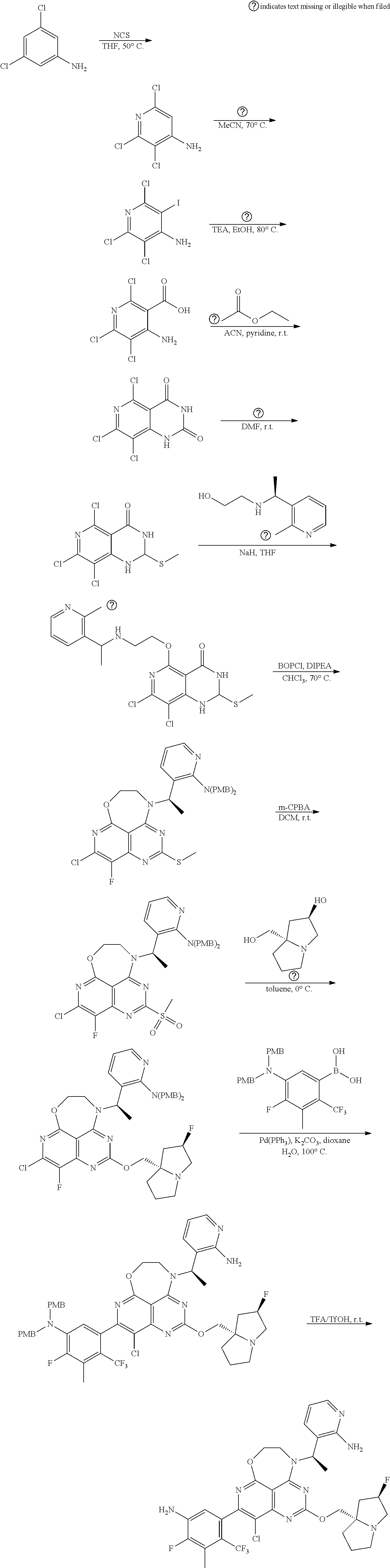

### Step 1: 2,3,6-trichloropyridin-4-amine

A solution of 2,6-dichloropyridin-4-amine (25.00 g, 153.37 mmol) and N-chlorosuccinimide (20.48 g, 153.33 mmol) in tetrahydrofuran (1000 mL) was stirred at 50° C. for 26 hours. After completion, the solvent was concentrated under vacuum. The resulting solution was diluted with water and extracted with ethyl acetate, the organic layers were combined and concentrated. Then the product was diluted with petroleum ether (200 mL) and ethyl acetate (20 mL). The resulting solution was stirred at room temperature for 0.5 h. After filtration, the solids were collected and washed with petroleum ether to afford 2,3,6-trichloropyridin-4-amine (26.70 g, 135.22 mmol, 88.2% yield) as a white solid. LC-MS: (ESI, m/Z): 197.0 [M+H]⁺

### Step 2: 2,3,6-trichloro-5-iodopyridin-4-amine

To a solution of 2,3,6-trichloropyridin-4-amine (25.40 g, 128.64 mmol) in acetonitrile (300 mL) was added N-iodosuccinimide (34.72 g, 154.33 mmol) and p-toluenesulfonic acid (2.22 g, 12.86 mmol) at 25° C. The resulting solution was stirred 2 hours at 70° C. After completion, the solvent was concentrated under vacuum. The resulting solution diluted with ethyl acetate and washed with water, the organic layers were combined and concentrated. The residue was purified by flash chromatography on silica gel eluting with petroleum ether/ethyl acetate (3:1) to afford 2,3,6-trichloro-5-iodopyridin-4-amine (38.1 g, 117.83 mmol, 91.6% yield) as a white solid. LC-MS: (ESI, m/Z): 322.8 [M+H]⁺

### Step 3:ethyl 4-amino-2,5,6-trichloronicotinate

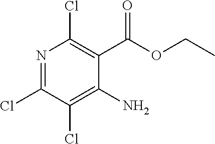

A mixture of 2,3,6-trichloro-5-iodopyridin-4-amine (35.30 g, 109.17 mmol), bis(triphenylphosphine)palladium(II) chloride (7.66 g, 10.91 mmol) and triethylamine (33.11 g, 327.20 mmol) in ethanol (350 mL) was stirred for 30 hours at 80° C. under carbon monoxide atmosphere. After completion, the resulting solution was diluted with ethyl acetate and washed with water. The organic layer was washed with brine and dried over anhydrous sodium sulfate and concentrated under vacuum. The residue was purified by flash chromatography on silica gel eluting with petroleum ether/ethyl acetate (4:1) to afford ethyl 4-amino-2,5,6-trichloronicotinate (21.30 g, 79.03 mmol, 72.4% yield) as an orange solid. LC-MS: (ESI, m/Z): 269.1 [M+H]⁺

### Step 4: 4-amino-2,5,6-trichloronicotinic acid

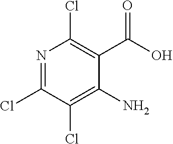 [SMILES: Nc1c(Cl)c(Cl)nc(Cl)c1C(=O)O]

A solution of ethyl 4-amino-2,5,6-trichloronicotinate (19.16 g, 71.08 mmol) and lithium hydroxide (3.41 g, 142.48 mmol) in methyl alcohol (120 mL), water (30 mL) and tetrahydrofuran (60 mL) was stirred at 25° C. for 1 hour. After completion, the resulting mixture was filtered. The filtrate was acidified to pH=3 with hydrochloric acid (6 mol/L). The resulting solution was diluted with water and extracted with ethyl acetate. The organic layers were combined, washed with brine, dried over anhydrous sodium sulfate and concentrated under vacuum to afford 4-amino-2,5,6-trichloronicotinic acid (17.00 g, 70.40 mmol, 99% yield) as a white solid. LC-MS: (ESI, m/Z): 242.8 [M+H]⁺

### Step 5:5,7,8-trichloro-2-thioxo-2,3-dihydropyrido[4,3-d]pyrimidin-4(1H)-one

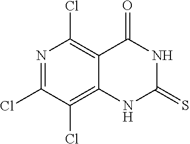 [SMILES: O=c1[nH]c(=S)[nH]c2c(Cl)c(Cl)nc(Cl)c12]

To a solution of 4-amino-2,5,6-trichloronicotinic acid (10.00 g, 41.41 mmol) in acetonitrile (90 mL) was added cyanic (ethyl carbonic) thioanhydride (21.80 g, 166.22 mmol) in pyridine (30 mL) at 0° C. The resulting solution was stirred at room temperature for 4 hours. After filtration, the solids were collected and washed with acetonitrile to afford 5,7,8-trichloro-2-thioxo-2,3-dihydropyrido[4,3-d]pyrimidin-4(1H)-one (7.41 g, 26.23 mmol, 63.3% yield) as a yellow solid. LC-MS: (ESI, m/Z): 279.8 [M−H]⁻

### Step 6: 5,7,8-trichloro-2-(methylthio)pyrido[4,3-d]pyrimidin-4(3H)-one

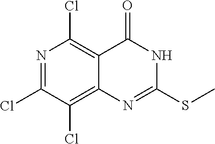 [SMILES: CSc1nc2c(Cl)c(Cl)nc(Cl)c2c(=O)[nH]1]

A solution of 5,7,8-trichloro-2-thioxo-2,3-dihydropyrido[4,3-d]pyrimidin-4(1H)-one (6.46 g, 22.87 mmol), sodium methoxide (1.11 g, 20.57 mmol) in N,N-dimethylformamide (65 mL) was stirred at room temperature for 1 hour. Then iodomethane (3.90 g, 27.45 mmol) was added and stirred at room temperature for 2 hours. After completion, the reaction mixture was diluted with water. The resulting solution was extracted with dichloromethane and the organic layers were combined. The organic layer was dried over anhydrous sodium sulfate and concentrated under vacuum to afford 5,7,8-trichloro-2-(methylthio)pyrido[4,3-d]pyrimidin-4(3H)-one (5.01 g,16.89 mmol, 73.9% yield) as a yellow solid. LC-MS: (ESI, m/Z): 295.9 [M+H]⁺

### Step 7:(R)-5-(2-((1-(2-(bis(4-methoxybenzyl)amino)pyridin-3-yl)ethyl)amino)ethoxy)-7,8-dichloro-2-(methylthio)pyrido[4,3-d]pyrimidin-4(3H)-one

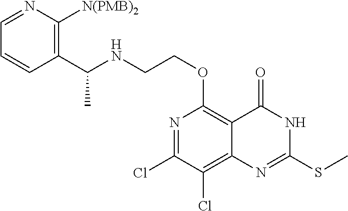

To a solution of (R)-2-((1-(2-(bis(4-methoxybenzyl)amino)pyridin-3-yl)ethyl)amino)ethan-1-ol (1.09 g, 2.58 mmol) in tetrahydrofuran (8 mL) was added sodium hydride (380.0 mg, 9.5 mmol, 60% dispersion in mineral oil) at 0° C. The resulting solution was stirred for 10 minutes at 0° C. Then 5,7,8-trichloro-2-(methylthio)pyrido[4,3-d]pyrimidin-4(3H)-one (700.0 mg, 2.36 mmol) was added and stirred at 25° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (9:1) to afford (R)-5-(2-((1-(2-(bis(4-methoxybenzyl)amino)pyridin-3-yl)ethyl)amino)ethoxy)-7,8-dichloro-2-(methylthio)pyrido[4,3-d]pyrimidin-4(3H)-one (1.52 g, 2.24 mmol, 94.7% yield) as an orange solid. LC-MS: (ESI, m/Z): 681.1 [M+H]⁺

### Step 8: (R)-3-(1-(4,5-dichloro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine

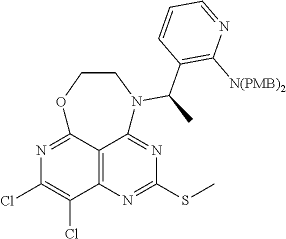

To a solution of (R)-5-(2-((1-(2-(bis(4-methoxybenzyl)amino)pyridin-3-yl)ethyl)amino)ethoxy)-7,8-dichloro-2-(methylthio)pyrido[4,3-d]pyrimidin-4(3H)-one (1.50 g, 2.20 mmol) in chloroform (15 mL) was added N,N-diisopropylethylamine (855.0 mg, 6.62 mmol) and bis(2-oxo-3-oxazolidinyl)phosphinic chloride (1.68 g, 6.60 mmol), the mixture was stirred at 70° C. for 1 hour. After completion, the resulting solution was diluted with dichloromethane and washed with water. The organic layer was washed with brine and dried over anhydrous sodium sulfate and concentrated under vacuum. The residue was purified by flash chromatography on silica gel eluting with dichloromethane/methanol (9:1) to afford (R)-3-(1-(4,5-dichloro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (900.0 mg, 1.35 mmol, 61.6% yield) as a yellow solid. LC-MS: (ESI, m/Z): 663.1 [M+H]⁺

### Step 9: (R)-3-(1-(4,5-dichloro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine

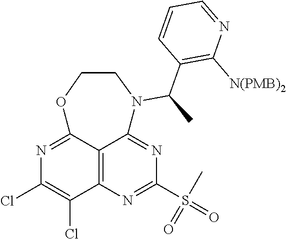

A solution of (R)-3-(1-(4,5-dichloro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (780.0 mg, 1.18 mmol) and m-chloro-peroxybenzoic acid (508.7 mg, 2.94 mmol) in dichloromethane (7.8 mL) was stirred at 25° C. for 1 hour. After completion, the reaction was quenched with saturated sodium sulfite solution. The reaction mixture was diluted with water and extracted with dichloromethane. The organic layers were dried over anhydrous sodium sulfate and concentrated under vacuum. The residue was purified by flash chromatography on silica gel eluting with dichloromethane/methanol (10:1) to afford (R)-3-(1-(4,5-dichloro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine (500.0 mg, 0.63 mmol, 54.3% yield) as a white solid. LC-MS: (ESI, m/Z): 695.6 [M+H]⁺

### Step 10: 3-((R)-1-(4,5-dichloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine

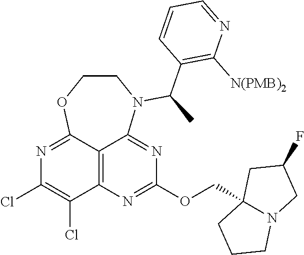

To a solution of ((2R,7aS)-2-fluorotetrahydro-1H-pyrrolizin-7a(5H)-yl)methanol (211.6 mg, 1.33 mmol) in toluene (5 mL) was added sodium tert-butoxide (190.4 mg, 1.19 mmol), the mixture was stirred at 0° C. for 20 minutes. Then (R)-3-(1-(4,5-dichloro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine (460.0 mg, 0.66 mmol) was added and stirred at 0° C. for 15 minutes. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (10:1) to afford 3-((R)-1-(4,5-dichloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (280.0 mg, 0.32 mmol, 48.6% yield) as a white solid. LC-MS: (ESI, m/Z): 774.7 [M+H]⁺

### Step 11: 3-((R)-1-(5-(5-(bis(4-methoxybenzyl)amino)-4-fluoro-3-methyl-2-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine

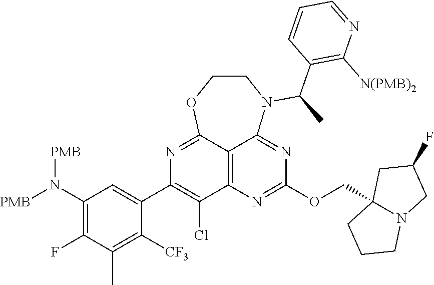

Under nitrogen, a solution of (5-(bis(4-methoxybenzyl)amino)-4-fluoro-3-methyl-2-(trifluoromethyl)phenyl)boronic acid (184.2 mg, 0.39 mmol), tetrakis(triphenylphosphine)palladium (34.2 mg, 0.03 mmol), potassium carbonate (122.9 mg, 0.89 mmol) and 3-((R)-1-(4,5-dichloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (230.0 mg, 0.30 mmol) in 1,4-dioxane (2.3 mL) and water (0.23 mL) was stirred at 100° C. for 1 hour. After completion, the reaction mixture was diluted with water. The resulting solution was extracted with ethyl acetate and the organic layers were combined. The organic layer was dried over anhydrous sodium sulfate and concentrated under vacuum. The residue was purified by flash chromatography on silica gel eluting with petroleum ether/ethyl acetate (3:1) to afford 3-((R)-1-(5-(5-(bis(4-methoxybenzyl)amino)-4-fluoro-3-methyl-2-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (130.0 mg, 0.10 mmol, 35.5% yield) as a white solid. LC-MS: (ESI, m/Z): 1171.7 [M+H]⁺

### Step 12: 3-((R)-1-(5-(5-amino-4-fluoro-3-methyl-2-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

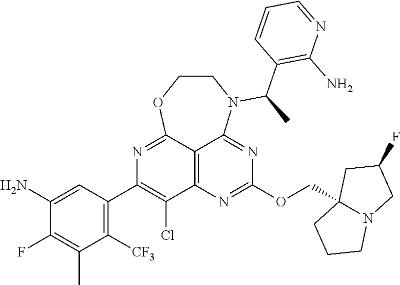 [SMILES: Cc1c(F)c(N)cc(-c2nc3c4c(nc(OC[C@@]56CCCN5C[C@H](F)C6)nc4c2Cl)N([C@H](C)c2cccnc2N)CCO3)c1C(F)(F)F]

A solution of 3-((R)-1-(5-(5-(bis(4-methoxybenzyl)amino)-4-fluoro-3-methyl-2-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (115.0 g, 0.10 mmol) in trifluoroacetic acid (1.0 mL) and trifluoromethanesulfonic (0.1 mL) was stirred at 25° C. for 15 minutes. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 33% B to 54% B in 10 min, 54% B; Wave Length: 254/220 nm; RT1(min): 9.8 to afford 3-((R)-1-(5-(5-amino-4-fluoro-3-methyl-2-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (19.6 g, 0.01 mmol, 17% yield) as a white solid. LC-MS: (ESI, m/Z): 691.3 [M+H]⁺

Example 32: ¹H NMR (300 MHz, DMSO-d₆) δ 8.03-7.92 (m, 1H), 7.63 (d, J=7.4 Hz, 1H), 6.73-6.63 (m, 1H), 6.38 (dd, J=14.0, 7.7 Hz, 2H), 5.98 (d, J=3.7 Hz, 2H), 5.71 (s, 2H), 5.30 (d, J=54.4 Hz, 1H), 4.53-4.37 (m, 1H), 4.37-4.25 (m, 1H), 4.23-4.07 (m, 2H), 3.80-3.64 (m, 1H), 3.42-3.39 (m, 1H), 3.18-3.00 (m, 3H), 2.89-2.73 (m, 1H), 2.32 (s, 3H), 2.23-2.13 (m, 1H), 2.11-1.96 (m, 2H), 1.91-1.72 (m, 3H), 1.56 (t, J=5.6 Hz, 3H).

### Example 601a: 4-((R)-10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-chloro-6-methyl-5-(trifluoromethyl)pyridin-2-amine

### Example 601b: 4-((S)-10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-chloro-6-methyl-5-(trifluoromethyl)pyridin-2-amine

### Synthetic Route

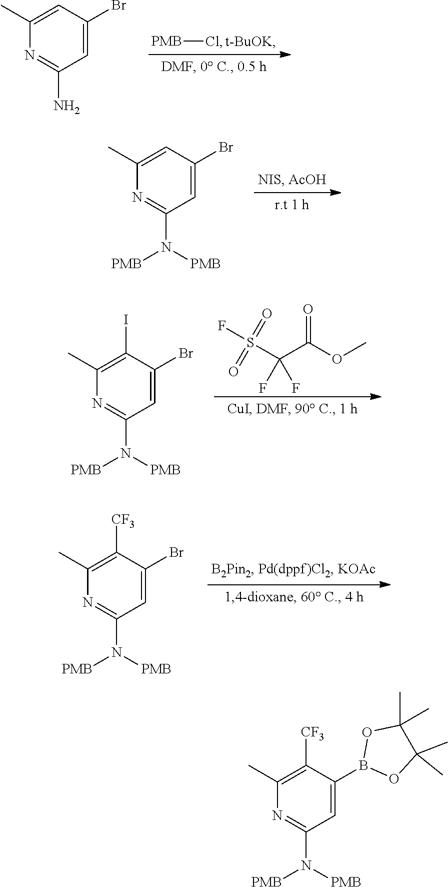

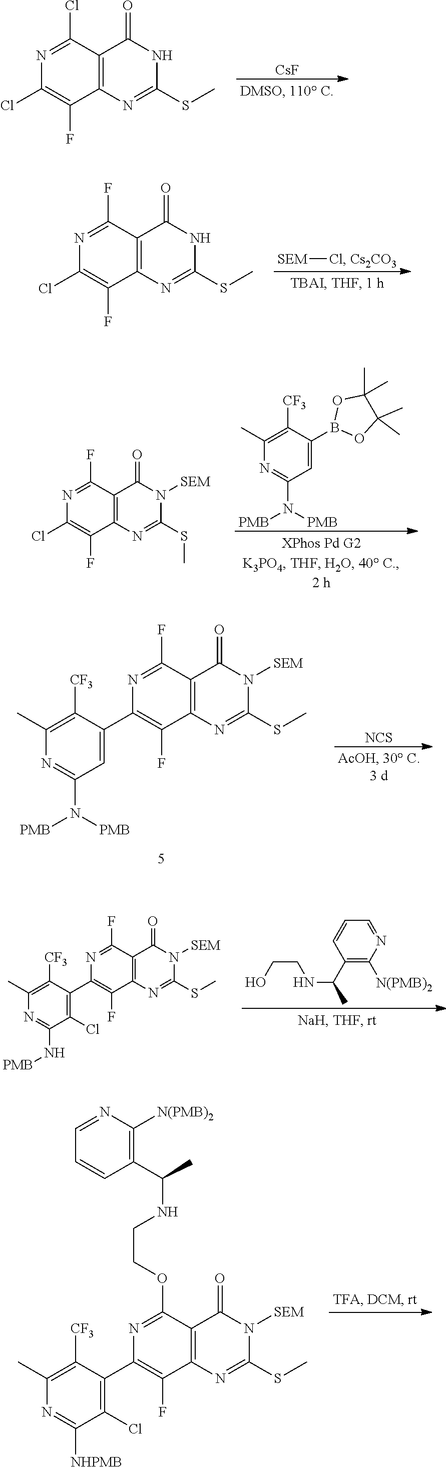

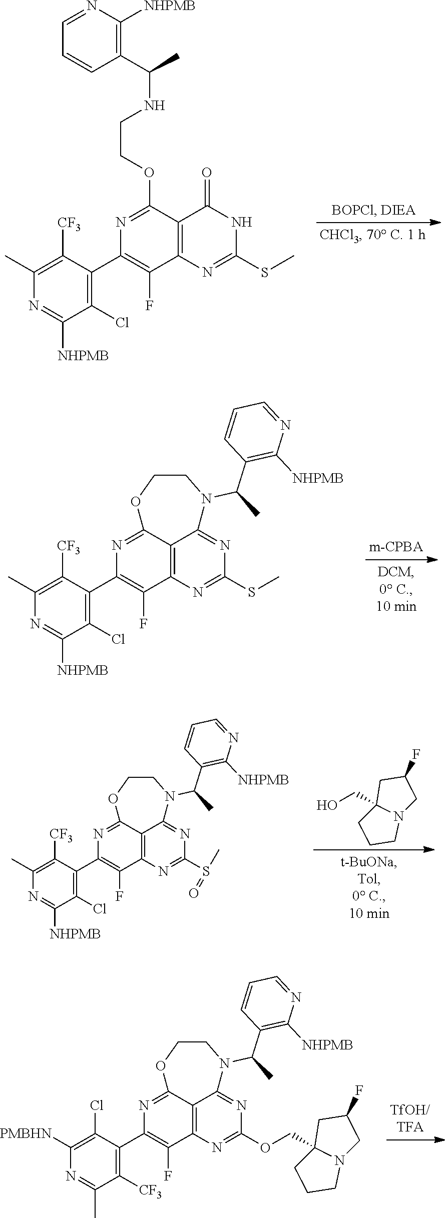

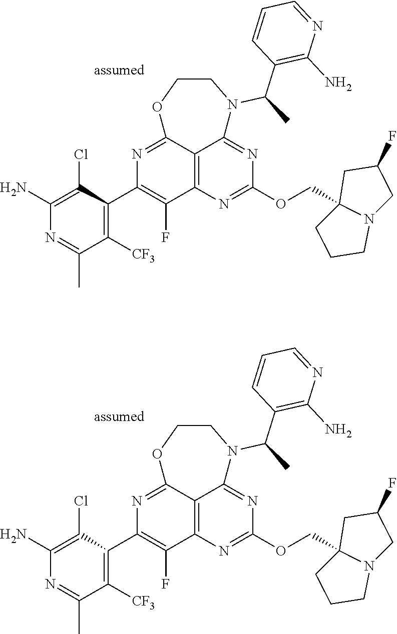

### Step 1: 4-bromo-N,N-bis(4-methoxybenzyl)-6-methylpyridin-2-amine

To a solution of 4-bromo-6-methyl-pyridin-2-amine (10.0 g, 53.46 mmol) in N,N-dimethylformamide (100 mL) was added 4-methoxybenzylchloride (29.19 g, 187.13 mmol) and potassium tert-butoxide (29.94 g, 267.32 mmol), the mixture was stirred at 0° C. for 1 hour. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (9/1) to afford 4-bromo-N,N-bis(4-methoxybenzyl)-6-methylpyridin-2-amine (24 g, 51.67 mmol, 96.6% yield) as a white solid. LCMS (ESI, m/z): 427.1/429.1 [M+H]⁺.

### Step 2: 4-bromo-5-iodo-N,N-bis(4-methoxybenzyl)-6-methylpyridin-2-amine

To a solution of 4-bromo-N,N-bis(4-methoxybenzyl)-6-methylpyridin-2-amine (30.0 g, 70.2 mmol) in acetic acid (300 mL) was added N-iodosuccinimide (11.06 g, 49.14 mmol), the mixture was stirred for 1 hour at room temperature. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with petroleum ether/ethyl acetate (9:1) to afford 4-bromo-5-iodo-N,N-bis(4-methoxybenzyl)-6-methylpyridin-2-amine (30 g, 50.43 mmol, 71.8% yield) as a yellow solid. LCMS (ESI, m/z): 523.0/525.0 [M+H]⁺.

### Step 3: 4-bromo-N,N-bis(4-methoxybenzyl)-6-methyl-5-(trifluoromethyl)pyridin-2-amine

Under nitrogen, a solution of 4-bromo-5-iodo-N,N-bis(4-methoxybenzyl)-6-methylpyridin-2-amine (29.0 g, 52.42 mmol), cuprous iodide (99.6 g, 524.19 mmol) and methyl 2,2-difluoro-2-(fluorosulfonyl)acetate (66.73 mL, 524.19 mmol) in N,N-dimethylformamide (300 mL) was stirred at 90° C. for 2 hours. After completion, the resulting solution was diluted with water and extracted with ethyl acetate. The organic layers were washed with brine, dried over anhydrous sodium sulfate and concentrated. The residue was purified by flash chromatography on silica gel eluting with dichloromethane/petroleum ether (1:1) to afford 4-bromo-N,N-bis(4-methoxybenzyl)-6-methyl-5-(trifluoromethyl)pyridin-2-amine (20 g, 38.76 mmol, 73.9% yield) as a yellow solid. LCMS (ESI, m/z): 495.1/497.1 [M+H]⁺.

### Step 4: N,N-bis(4-methoxybenzyl)-6-methyl-4-(4,4,5,5-tetramethyl-1,3,2-dioxaborolan-2-yl)-5-(trifluoromethyl)pyridin-2-amine

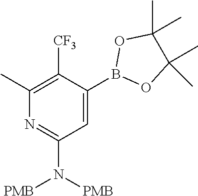

Under nitrogen, a solution of 4-bromo-N,N-bis(4-methoxybenzyl)-6-methyl-5-(trifluoromethyl)pyridin-2-amine (3.78 g, 7.63 mmol), bis(pinacolato)diboron (11.6 g, 45.78 mmol), [1,1′-bis(diphenylphosphino)ferrocene]dichloropalladium(II) (566.1 mg, 0.76 mmol) and potassium acetate (1.49 g, 15.26 mmol) in 1,4-dioxane (38 mL) was stirred at 60° C. for 4 hours. After completion, the solids were filtered out. The filtrate was concentrated under reduced pressure. The resulting residue was purified by reverse phase chromatography (acetonitrile 80-85/0.1% NH₄HCO₃ in water) to afford N,N-bis(4-methoxybenzyl)-6-methyl-4-(4,4,5,5-tetramethyl-1,3,2-dioxaborolan-2-yl)-5-(trifluoromethyl)pyridin-2-amine (871 mg, 1.60 mmol, 21% yield) as a yellow oil. LCMS (ESI, m/z):543.3 [M+H]⁺.

### Step 5: 7-chloro-5,8-difluoro-2-(methylthio)pyrido[4,3-d]pyrimidin-4(3H)-one

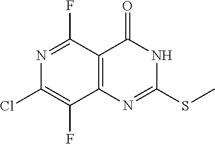 [SMILES: CSc1nc2c(F)c(Cl)nc(F)c2c(=O)[nH]1]

Under nitrogen, a mixture of 5,7-dichloro-8-fluoro-2-methylsulfanyl-3H-pyrido[4,3-d]pyrimidin-4-one (3.00 g, 10.71 mmol) and caesium fluoride (16.30 g, 107.31 mmol) in dimethyl sulfoxide (35 mL) was stirred at 110° C. for 16 hours. After completion, the reaction was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (3:1) to afford 7-chloro-5,8-difluoro-2-(methylthio)pyrido[4,3-d]pyrimidin-4(3H)-one (2.60 g, 8.38 mmol, 78.3% yield) as a light yellow solid. LC-MS: (ESI, m/z): 264.0 [M+H]⁺.

### Step 6: 7-chloro-5,8-difluoro-2-(methylthio)-3-((2-(trimethylsilyl)ethoxy)methyl)pyrido[4,3-d]pyrimidin-4(3H)-one

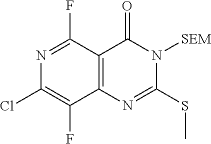 [SMILES: CSc1nc2c(F)c(Cl)nc(F)c2c(=O)n1COCC[Si](C)(C)C]

To a solution of 7-chloro-5,8-difluoro-2-(methylthio)pyrido[4,3-d]pyrimidin-4(3H)-one (2.60 g, 9.86 mmol) in tetrahydrofuran (30 mL) was added cesium carbonate (8.03 g, 24.65 mmol), tetrabutylammonium iodide (364.2 mg, 0.99 mmol) and 2-(trimethylsilyl)ethoxymethyl chloride (3.49 mL, 19.72 mmol), the resulting solution was stirred at room temperature for 1 hour. After completion, the reaction was diluted with water and extracted with dichloromethane. The organic layers were combined, washed with brine, dried over anhydrous sodium sulfate and concentrated. The residue was purified by flash chromatography on silica gel eluting with petroleum ether/ethyl acetate (2:1) to afford 7-chloro-5,8-difluoro-2-(methylthio)-3-((2-(trimethylsilyl)ethoxy)methyl)pyrido[4,3-d]pyrimidin-4(3H)-one (1.90 g, 4.05 mmol, 41.1% yield) as a white solid. LC-MS: (ESI, m/z): 394.1 [M+H]⁺

### Step 7: 7-(6-(bis(4-methoxybenzyl)amino)-2-methyl-3-(trifluoromethyl)pyridin-4-yl)-5,8-difluoro-2-(methylthio)-3-((2-(trimethylsilyl)ethoxy)methyl)pyrido[4,3-d]pyrimidin-4(3H)-one

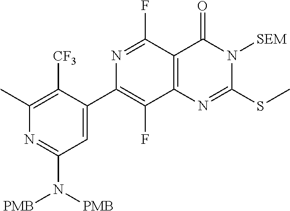

Under nitrogen, a mixture of 7-chloro-5,8-difluoro-2-(methylthio)-3-((2-(trimethylsilyl)ethoxy)methyl)pyrido[4,3-d]pyrimidin-4(3H)-one (800.0 mg, 2.03 mmol), N,N-bis(4-methoxybenzyl)-6-methyl-4-(4,4,5,5-tetramethyl-1,3,2-dioxaborolan-2-yl)-5-(trifluoromethyl)pyridin-2-amine (1.32 g, 2.44 mmol), XPhos Pd G2 (479.0 mg, 0.60 mmol) and potassium phosphate (1.29 g, 6.09 mmol) in tetrahydrofuran (8 mL) and water (0.8 mL) was stirred at 40° C. for 2 hours. After completion, the reaction was diluted with water and extracted with ethyl acetate. The organic layers were combined, dried over anhydrous sodium sulfate and concentrated. The residue was purified by flash chromatography on silica gel eluting with petroleum ether/ethyl acetate (7:3) to afford 7-(6-(bis(4-methoxybenzyl)amino)-2-methyl-3-(trifluoromethyl)pyridin-4-yl)-5,8-difluoro-2-(methylthio)-3-((2-(trimethylsilyl)ethoxy)methyl)pyrido[4,3-d]pyrimidin-4(3H)-one (346 mg, 0.45 mmol, 22% yield) as a yellow oil. LC-MS: (ESI, m/z): 774.2 [M+H]⁺

### Step 8: 7-(3-chloro-2-((4-methoxybenzyl)amino)-6-methyl-5-(trifluoromethyl)pyridin-4-yl)-5,8-difluoro-2-(methylthio)-3-((2-(trimethylsilyl)ethoxy)methyl)pyrido[4,3-d]pyrimidin-4(3H)-one

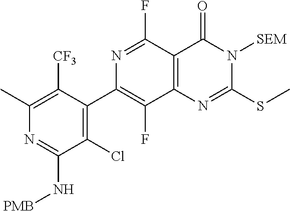

A mixture of 7-(6-(bis(4-methoxybenzyl)amino)-2-methyl-3-(trifluoromethyl)pyridin-4-yl)-5,8-difluoro-2-(methylthio)-3-((2-(trimethylsilyl)ethoxy)methyl)pyrido[4,3-d]pyrimidin-4(3H)-one (400.0 mg, 0.52 mmol) and N-chlorosuccinimide (207.0 mg, 1.55 mmol) in acetic acid (5 mL) was stirred at 30° C. for 3 days. After completion, the solvent was removed under vacuum. The reaction mixture was adjusted to pH 7 with saturated sodium bicarbonate solution. The resulting solution was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (4:1) to afford 7-(3-chloro-2-((4-methoxybenzyl)amino)-6-methyl-5-(trifluoromethyl)pyridin-4-yl)-5,8-difluoro-2-(methylthio)-3-((2-(trimethylsilyl)ethoxy)methyl)pyrido[4,3-d]pyrimidin-4(3H)-one (170 mg, 0.24 mmol, 47.8% yield) as a yellow solid. LC-MS: (ESI, m/z): 688.2 [M+H]⁺

### Step 9: 5-(2-(((R)-1-(2-(bis(4-methoxybenzyl)amino)pyridin-3-yl)ethyl)amino)ethoxy)-7-(3-chloro-2-((4-methoxybenzyl)amino)-6-methyl-5-(trifluoromethyl)pyridin-4-yl)-8-fluoro-2-(methylthio)-3-((2-(trimethylsilyl)ethoxy)methyl)pyrido[4,3-d]pyrimidin-4(3H)-one

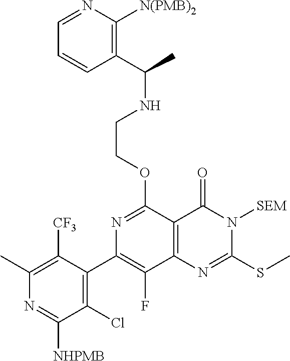

Under nitrogen, to a mixture of (R)-2-((1-(2-(bis(4-methoxybenzyl)amino)pyridin-3-yl)ethyl)amino)ethan-1-ol (367.0 mg, 0.87 mmol) in tetrahydrofuran (5 mL) was added sodium hydride (116.0 mg, 2.91 mmol, 60% dispersion in mineral oil), the mixture was stirred for 10 minutes at 0° C. Then the mixture was transferred into a solution of 7-(3-chloro-2-((4-methoxybenzyl)amino)-6-methyl-5-(trifluoromethyl)pyridin-4-yl)-5,8-difluoro-2-(methylthio)-3-((2-(trimethylsilyl)ethoxy)methyl)pyrido[4,3-d]pyrimidin-4(3H)-one (500.0 mg, 0.73 mmol) in tetrahydrofuran (5 mL), the mixture was stirred for 1 hour at room temperature. After completion, the reaction was quenched with saturated ammonium chloride solution. The resulting solution was diluted with water and extracted with ethyl acetate. The organic layers were combined, washed with brine, dried over anhydrous sodium sulfate and concentrated. The crude product would be directly used in the next step without purification. LC-MS: (ESI, m/z): 1089.4 [M+H]⁺

### Step 10: 7-(3-chloro-2-((4-methoxybenzyl)amino)-6-methyl-5-(trifluoromethyl)pyridin-4-yl)-8-fluoro-5-(2-(((R)-1-(2-((4-methoxybenzyl)amino)pyridin-3-yl)ethyl)amino)ethoxy)-2-(methylthio)pyrido[4,3-d]pyrimidin-4(3H)-one

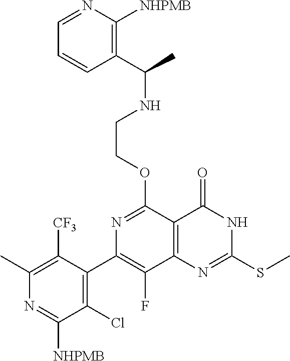

A mixture of 5-(2-(((R)-1-(2-(bis(4-methoxybenzyl)amino)pyridin-3-yl)ethyl)amino)ethoxy)-7-(3-chloro-2-((4-methoxybenzyl)amino)-6-methyl-5-(trifluoromethyl)pyridin-4-yl)-8-fluoro-2-(methylthio)-3-((2-(trimethylsilyl)ethoxy)methyl)pyrido[4,3-d]pyrimidin-4(3H)-one (700.0 mg, 0.64 mmol) in dichloromethane (10 mL) and 2,2,2-trifluoroacetic acid (2 mL) was stirred at room temperature for 2 hours. After completion, the solvent was removed under vacuum. The residue was purified by flash chromatography on silica gel eluting with dichloromethane/methanol (24:1) to afford 7-(3-chloro-2-((4-methoxybenzyl)amino)-6-methyl-5-(trifluoromethyl)pyridin-4-yl)-8-fluoro-5-(2-(((R)-1-(2-((4-methoxybenzyl)amino)pyridin-3-yl)ethyl)amino)ethoxy)-2-(methylthio)pyrido[4,3-d]pyrimidin-4(3H)-one (350 mg, 0.42 mmol, 64.9% yield) as a yellow solid. LC-MS: (ESI, m/z): 839.2 [M+H]⁺

### Step 11: 3-chloro-4-(4-fluoro-10-((R)-1-(2-((4-methoxybenzyl)amino)pyridin-3-yl)ethyl)-2-(methylthio)-9,10-dihydro-8H-7-oxa-1,3,6,10-tetraazacyclohepta[de]naphthalen-5-yl)-N-(4-methoxybenzyl)-6-methyl-5-(trifluoromethyl)pyridin-2-amine

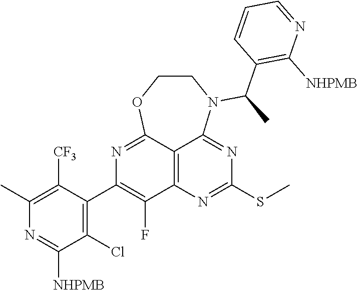

A mixture of bis(2-oxo-3-oxazolidinyl)phosphinicchloride (159.2 mg, 0.63 mmol), 7-(3-chloro-2-((4-methoxybenzyl)amino)-6-methyl-5-(trifluoromethyl)pyridin-4-yl)-8-fluoro-5-(2-(((R)-1-(2-((4-methoxybenzyl)amino)pyridin-3-yl)ethyl)amino)ethoxy)-2-(methylthio)pyrido[4,3-d]pyrimidin-4(3H)-one (350.0 mg, 0.42 mmol) and N,N-diisopropylethylamine (269.4 mg, 2.09 mmol) in chloroform (4 mL) was stirred at 70° C. for 1 hour. After completion, the reaction was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (1:1) to afford 3-chloro-4-(4-fluoro-10-((R)-1-(2-((4-methoxybenzyl)amino)pyridin-3-yl)ethyl)-2-(methylthio)-9,10-dihydro-8H-7-oxa-1,3,6,10-tetraazacyclohepta[de]naphthalen-5-yl)-N-(4-methoxybenzyl)-6-methyl-5-(trifluoromethyl)pyridin-2-amine (180 mg, 0.22 mmol, 52.6% yield) as a yellow solid. LC-MS: (ESI, m/z): 821.2 [M+H]⁺

### Step 12: 3-chloro-4-(4-fluoro-10-((R)-1-(2-((4-methoxybenzyl)amino)pyridin-3-yl)ethyl)-2-(methylsulfinyl)-9,10-dihydro-8H-7-oxa-1,3,6,10-tetraazacyclohepta[de]naphthalen-5-yl)-N-(4-methoxybenzyl)-6-methyl-5-(trifluoromethyl)pyridin-2-amine

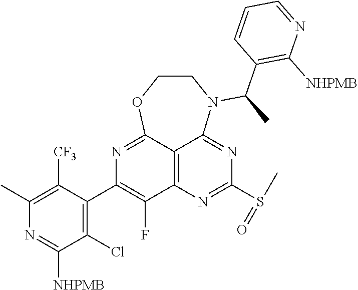

To a mixture of 3-chloro-4-(4-fluoro-10-((R)-1-(2-((4-methoxybenzyl)amino)pyridin-3-yl)ethyl)-2-(methylthio)-9,10-dihydro-8H-7-oxa-1,3,6,10-tetraazacyclohepta[de]naphthalen-5-yl)-N-(4-methoxybenzyl)-6-methyl-5-(trifluoromethyl)pyridin-2-amine (180.0 mg, 0.22 mmol) in dichloromethane (2 mL) was added 3-chloroperoxybenzoicacid (56.7 mg, 0.33 mmol), the mixture was stirred for 10 minutes at 0° C. After completion, the reaction was quenched with saturated sodium sulfite solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (49:1) to afford 3-chloro-4-(4-fluoro-10-((R)-1-(2-((4-methoxybenzyl)amino)pyridin-3-yl)ethyl)-2-(methylsulfinyl)-9,10-dihydro-8H-7-oxa-1,3,6,10-tetraazacyclohepta[de]naphthalen-5-yl)-N-(4-methoxybenzyl)-6-methyl-5-(trifluoromethyl)pyridin-2-amine (110 mg, 0.13 mmol, 59.9% yield) as a yellow solid. LC-MS: (ESI, m/z): 837.2 [M+H]⁺

### Step 13: 3-chloro-4-(4-fluoro-2-(((2R,7aS)-2-fluorotetrahydro-1H-pyrrolizin-7a(5H)-yl)methoxy)-10-((R)-1-(2-((4-methoxybenzyl)amino)pyridin-3-yl)ethyl)-9,10-dihydro-8H-7-oxa-1,3,6,10-tetraazacyclohepta[de]naphthalen-5-yl)-N-(4-methoxybenzyl)-6-methyl-5-(trifluoromethyl)pyridin-2-amine

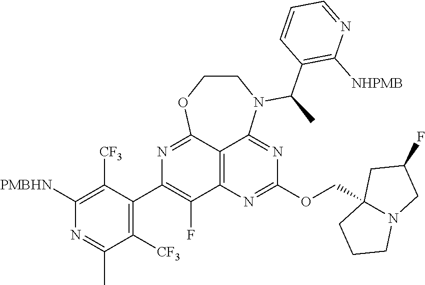

To a mixture of [(2R,8S)-2-fluoro-1,2,3,5,6,7-hexahydropyrrolizin-8-yl]methanol (41.8 mg, 0.26 mmol) in toluene (1 mL) was added sodium tert-butoxide (37.9 mg, 0.39 mmol), the mixture was stirred for 10 minutes at 0° C. Then 3-chloro-4-(4-fluoro-10-((R)-1-(2-((4-methoxybenzyl)amino)pyridin-3-yl)ethyl)-2-(methylsulfinyl)-9,10-dihydro-8H-7-oxa-1,3,6,10-tetraazacyclohepta[de]naphthalen-5-yl)-N-(4-methoxybenzyl)-6-methyl-5-(trifluoromethyl)pyridin-2-amine (110.0 mg, 0.13 mmol) in toluene (0.2 mL) was added, stirred for 10 minutes at 0° C. After completion, the reaction was quenched with saturated ammonium chloride solution. The resulting solution was diluted with water and extracted with ethyl acetate. The organic layers were combined, washed with brine, dried over anhydrous sodium sulfate and concentrated. The resulting residue was purified by reverse phase chromatography (acetonitrile/0.1% ammonium bicarbonate in water) to afford 3-chloro-4-(4-fluoro-2-(((2R,7aS)-2-fluorotetrahydro-1H-pyrrolizin-7a(5H)-yl)methoxy)-10-((R)-1-(2-((4-methoxybenzyl)amino)pyridin-3-yl)ethyl)-9,10-dihydro-8H-7-oxa-1,3,6,10-tetraazacyclohepta[de]naphthalen-5-yl)-N-(4-methoxybenzyl)-6-methyl-5-(trifluoromethyl)pyridin-2-amine (60 mg, 0.06 mmol, 49% yield) as a white solid. LC-MS: (ESI, m/z): 932.3 [M+H]⁺

### Step 14: 4-((R)-10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-chloro-6-methyl-5-(trifluoromethyl)pyridin-2-amine & 4-((S)-10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-chloro-6-methyl-5-(trifluoromethyl)pyridin-2-amine

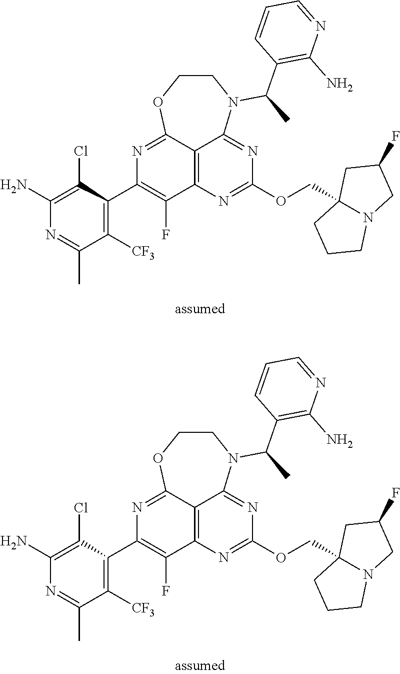

A mixture of 3-chloro-4-(4-fluoro-2-(((2R,7aS)-2-fluorotetrahydro-1H-pyrrolizin-7a(5H)-yl)methoxy)-10-((R)-1-(2-((4-methoxybenzyl)amino)pyridin-3-yl)ethyl)-9,10-dihydro-8H-7-oxa-1,3,6,10-tetraazacyclohepta[de]naphthalen-5-yl)-N-(4-methoxybenzyl)-6-methyl-5-(trifluoromethyl)pyridin-2-amine (90.0 mg, 0.10 mmol) in trifluoroacetic acid (1 mL) and trifuoromethanesukfonic acid (0.1 mL) was stirred at room temperature for 0.5 hours. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (9:1). The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 31% B to 55% B in 9 min; Wave Length: 254 nm/220 nm; RT1(min): 8, RT2(min): 9 to afford 4-((R)-10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-chloro-6-methyl-5-(trifluoromethyl)pyridin-2-amine (8.7 mg, 0.01 mmol, 13% yield) as a white solid and 4-((S)-10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-chloro-6-methyl-5-(trifluoromethyl)pyridin-2-amine (9.7 mg, 0.01 mmol, 14.5% yield) as a white solid. The stereo chemistry of title compounds was arbitrarily assigned.

Example 601a: LCMS (ESI, m/z):692.4 [M+H]⁺. ¹H NMR (300 MHz, Methanol-d₄) δ 7.96 (dd, J=5.1, 1.7 Hz, 1H), 7.76-7.73 (m, 1H), 6.78 (dd, J=7.5, 5.1Hz, 1H), 6.60 (q, J=6.8 Hz, 1H), 5.27 (d, J=52.2 Hz, 1H), 4.46 (dd, J=12.8, 6.2 Hz, 1H), 4.38-4.32 (m, 3H), 3.76 (dd, J=16.0, 6.5 Hz, 1H), 3.54 (dd, J=16.0, 6.3 Hz, 1H), 3.24-3.16 (m, 3H), 3.02-2.93 (m, 1H), 2.54 (s, 3H), 2.37-2.13 (m, 3H), 2.03-1.96 (m, 3H), 1.64 (d, J=6.9 Hz, 3H).

Example 601b: LCMS (ESI, m/z):692.3 [M+H]⁺. ¹H NMR (300 MHz, Methanol-d₄) δ 7.96 (dd, J=5.1, 1.6 Hz, 1H), 7.76-7.74 (m, 1H), 6.78 (dd, J=7.5, 5.1Hz, 1H), 6.60 (q, J=6.9 Hz, 1H), 5.31 (d, J=52.2 Hz, 1H), 4.48 (dd, J=12.8, 6.2 Hz, 1H), 4.36-4.30 (m, 3H), 3.76 (dd, J=16.1, 6.5 Hz, 1H), 3.55 (dd, J=16.1, 6.2 Hz, 1H), 3.24-3.17 (m, 3H), 3.03-2.92 (m, 1H), 2.54 (s, 3H), 2.37-2.12 (m, 3H), 2.03-1.95 (m, 3H), 1.65 (d, J=6.9 Hz, 3H).

### Example 602: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)quinolin-2-amine

### Synthetic Route

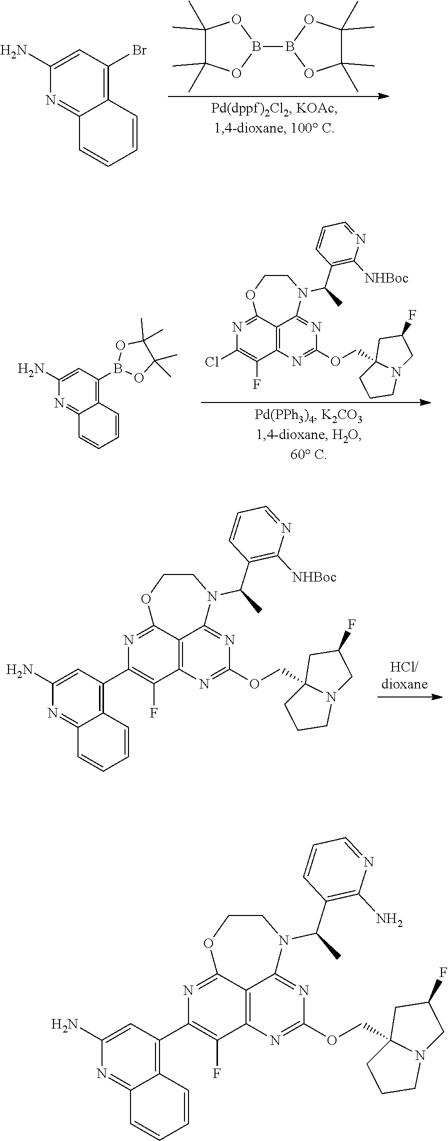

### Step 1: 4-(4,4,5,5-tetramethyl-1,3,2-dioxaborolan-2-yl)quinolin-2-amine

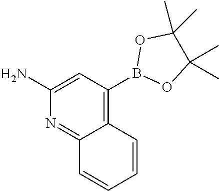 [SMILES: CC1(C)OB(c2cc(N)nc3ccccc23)OC1(C)C]

Under nitrogen, a solution of 4-bromoquinolin-2-amine (550.0 mg, 2.47 mmol), bis(pinacolato)diboron (1.25 g, 4.93 mmol), [1,1′-bis(diphenylphosphino)ferrocene]dichloropalladium(II) (183.0 mg, 0.25 mmol) and potassium acetate (726.2 mg, 7.40 mmol) in 1,4-dioxane (5 mL) was stirred for 2 hours at 100° C. After completion, the reaction solution was diluted with water and extracted with ethyl acetate. The organic layers were washed with brine, dried over anhydrous sodium sulfate and concentrated. The resulting residue was purified by reverse phase chromatography (acetonitrile/0.1% NH₄HCO₃ in water) to afford 4-(4,4,5,5-tetramethyl-1,3,2-dioxaborolan-2-yl)quinolin-2-amine (180.0 mg,0.67 mmol, 27% yield) as a white solid. LC-MS: (ESI, m/z): 271.2 [M+H]⁺.

### Step 2: tert-butyl (3-((R)-1-(5-(2-aminoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate

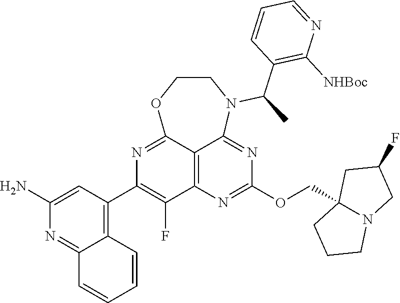 [SMILES: C[C@H](c1cccnc1NC(=O)OC(C)(C)C)N1CCOc2nc(-c3cc(N)nc4ccccc34)c(F)c3nc(OC[C@@]45CCCN4C[C@H](F)C5)nc1c23]

Under nitrogen, a solution of 4-(4,4,5,5-tetramethyl-1,3,2-dioxaborolan-2-yl)quinolin-2-amine (218.5 mg, 0.81 mmol), tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (250.0 mg, 0.40 mmol), potassium carbonate (167.7 mg, 1.21 mmol) and tetrakis(triphenylphosphine)palladium (46.7 mg, 0.04 mmol) in 1,4-dioxane (2 mL) and water (0.40 mL) was stirred at 60° C. for 8 hours. After completion, the resulting solution was diluted with water and extracted with ethyl acetate. The organic layers were washed with brine, dried over anhydrous sodium sulfate and concentrated under vacuum to afford tert-butyl (3-((R)-1-(5-(2-aminoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (457.0 mg, 0.37 mmol, 91.8% yield). The crude product would be directly used in the next step without purification. LC-MS: (ESI, m/z): 726.4 [M+H]⁺.

### Step 3: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)quinolin-2-amine

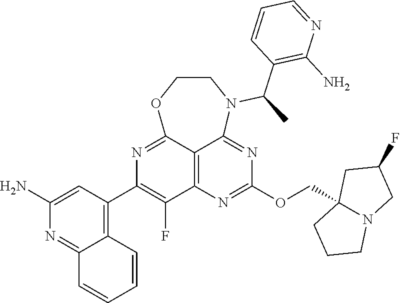 [SMILES: C[C@H](c1cccnc1N)N1CCOc2nc(-c3cc(N)nc4ccccc34)c(F)c3nc(OC[C@@]45CCCN4C[C@H](F)C5)nc1c23]

A solution of tert-butyl (3-((R)-1-(5-(2-aminoquinolin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (447.0 mg, 0.62 mmol) in 4 M hydrochloric acid in 1,4-dioxane (1 mL) was stirred at 25° C. for 1 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (10:1) to afford crude solid. The crude product was purified by Prep-HPLC with the following conditions: Column: YMC Triart C18 ExRs Sum, 30*150 mm; Mobile Phase A: Water(10 mmol/L NH₄HCO₃), Mobile Phase B: ACN; Flow rate: 60 mL/min; Gradient: 26% B to 40% B in 13 min; Wave Length: 254 nm/220 nm to afford 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)quinolin-2-amine (69.2 mg, 0.11 mmol, 18% yield) as a white solid. LC-MS: (ESI, m/z): 626.2 [M+H]⁺.

Example 602: ¹H NMR (300 MHz, DMSO-d₆, ppm) δ 7.98 (dd, J=4.9, 1.6 Hz, 1H), 7.64 (dd, J=7.5, 1.8 Hz, 1H), 7.56-7.44 (m, 3H), 7.15-7.10 (m, 1H), 6.86 (s, 1H), 6.68 (dd, J=7.5, 4.9 Hz, 1H), 6.61 (s, 2H), 6.37 (q, J=6.8 Hz, 1H), 5.73 (s, 2H), 5.29 (d, J=54.1Hz, 1H), 4.46 (dd, J=12.7, 6.5 Hz, 1H), 4.33 (dd, J=12.6, 6.3 Hz, 1H), 4.15 (s, 2H), 3.77 (dd, J=15.9, 6.4 Hz, 1H), 3.42 (dd, J=15.9, 6.5 Hz, 1H), 3.12-2.92 (m, 3H), 2.86-2.79 (m, 1H), 2.23-2.14 (m, 1H), 2.10-1.97 (m, 2H), 1.88-1.73 (m, 3H), 1.58 (d, J=6.8 Hz, 3H). LC-MS: (ESI, m/z): 626.2 [M+H]⁺.

### Example 604: 6-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-4-methyl-5-(trifluoromethyl)pyridin-2-amine

### Synthetic Route

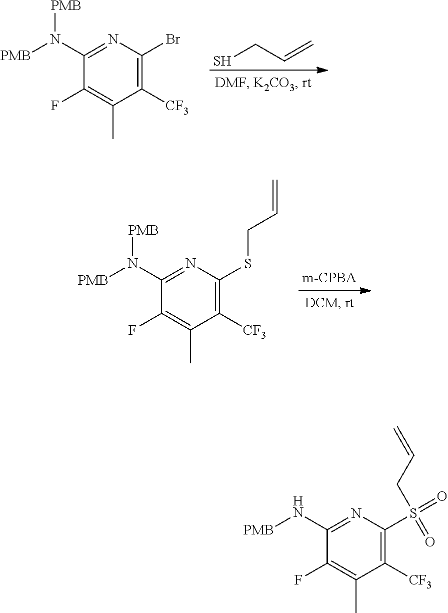

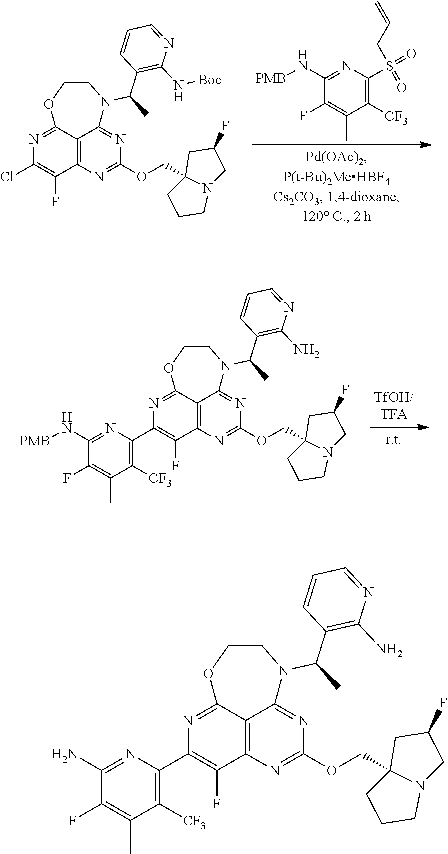

### Step 1: 6-(allylthio)-3-fluoro-N,N-bis(4-methoxybenzyl)-4-methyl-5-(trifluoromethyl)pyridin-2-amine

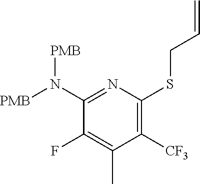

Under nitrogen, to a solution of 6-bromo-3-fluoro-N,N-bis[(4-methoxyphenyl)methyl]-4-methyl-5-(trifluoromethyl)pyridin-2-amine (1.3 g, 2.44 mmol) in N,N-dimethylformamide (10 mL) was added potassium carbonate (0.7 g, 4.87 mmol) and allyl mercaptan (0.97 ml, 12.18 mmol), the resulting solution was stirred at 25° C. for 24 hours. After completion, the reaction solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (11:1) to afford 6-(allylthio)-3-fluoro-N,N-bis(4-methoxybenzyl)-4-methyl-5-(trifluoromethyl)pyridin-2-amine (1.0 g, 1.72 mmol, 70.5% yield) as a light yellow oil. LC-MS: (ESI, m/z): 507.2 [M+H]⁺.

### Step 2: 6-(allylsulfonyl)-3-fluoro-N-(4-methoxybenzyl)-4-methyl-5-(trifluoromethyl)pyridin-2-amine

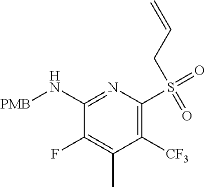

To a solution of 6-(allylthio)-3-fluoro-N,N-bis(4-methoxybenzyl)-4-methyl-5-(trifluoromethyl)pyridin-2-amine (3.3 g, 6.42 mmol) in dichloromethane (30 mL) was added 3-chloroperoxybenzoic acid (5.5 g, 32.08 mmol) at 0° C. Then the mixture was stirred at 25° C. for 2 hours. The reaction was quenched with saturated sodium sulfite solution. The resulting solution was extracted with dichloromethane and the organic layers were combined. The organic layer was dried over anhydrous sodium sulfate and concentrated under vacuum. The residue was purified by flash chromatography on silica gel eluting with petroleum ether/ethyl acetate (5:1) to afford 6-(allylsulfonyl)-3-fluoro-N-(4-methoxybenzyl)-4-methyl-5-(trifluoromethyl)pyridin-2-amine (2.8 g, 4.47 mmol, 69.7% yield) as a white solid. LC-MS: (ESI, m/z): 419.2 [M+H]⁺.

### Step 3: 6-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-N-(4-methoxybenzyl)-4-methyl-5-(trifluoromethyl)pyridin-2-amine

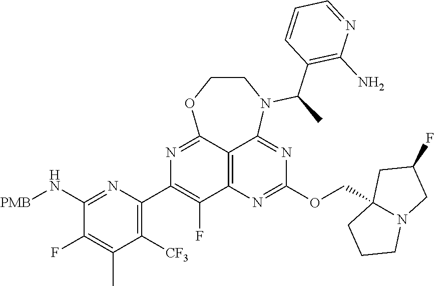

Under nitrogen, a solution of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (150.0 mg, 0.24 mmol), 6-(allylsulfonyl)-3-fluoro-N-(4-methoxybenzyl)-4-methyl-5-(trifluoromethyl)pyridin-2-amine (101.5 mg, 0.24 mmol), palladium(II)acetate (16.5 mg, 0.07 mmol), di-tert-butylmethylphosphonium tetrafluoroborate (36.0 mg, 0.15 mmol) and caesium carbonate (237.2 mg, 0.73 mmol) in 1,4-dioxane (2 mL) was stirred at 120° C. for 2 hours. After completion, the reaction was extracted with ethyl acetate and the organic layers was washed with brine and the organic layer was combined. The organic layer was concentrated under vacuum and purified by flash chromatography on silica gel eluting with dichloromethane/methanol (8:1) to afford 6-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-N-(4-methoxybenzyl)-4-methyl-5-(trifluoromethyl)pyridin-2-amine (109.0 mg, 0.09 mmol, 38.4% yield) as a yellow solid. LC-MS: (ESI, m/z): 796.3 [M+H]⁺.

### Step 4: 6-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-4-methyl-5-(trifluoromethyl)pyridin-2-amine

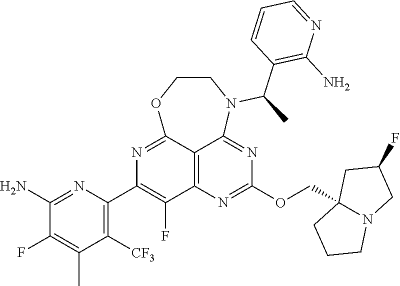 [SMILES: Cc1c(F)c(N)nc(-c2nc3c4c(nc(OC[C@@]56CCCN5C[C@H](F)C6)nc4c2F)N([C@H](C)c2cccnc2N)CCO3)c1C(F)(F)F]

A solution of 6-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-N-(4-methoxybenzyl)-4-methyl-5-(trifluoromethyl)pyridin-2-amine (109.0 mg, 0.14 mmol) in trifluoromethanesulfonic acid (0.1 mL) and 2,2,2-trifluoroacetic acid (1 mL) was stirred at 25° C. for 1 hour. After completion, the reaction mixture was diluted with dichloromethane and adjusted to pH>7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (9:1) to afford crude product. The resulting residue was purified by reverse phase chromatography (acetonitrile/0.1% NH₄HCO₃ in water) to afford 6-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-4-methyl-5-(trifluoromethyl)pyridin-2-amine (11.7 mg, 0.02 mmol, 12.6% yield) as a white solid. LC-MS: (ESI, m/z): 676.2 [M+H]⁺.

Example 604: ¹H NMR (400 MHz, DMSO-d₆, ppm) δ 7.97 (dd, J=4.9, 1.7 Hz, 1H), 7.63 (dd, J=7.5, 1.8 Hz, 1H), 7.16 (s, 2H), 6.67 (dd, J=7.5, 4.9 Hz, 1H), 6.33 (q, J=6.8 Hz, 1H), 5.70 (s, 2H), 5.29 (d, J=53.4 Hz, 1H), 4.42 (dd, J=12.7, 6.6 Hz, 1H), 4.30 (dd, J=12.7, 6.5 Hz, 1H), 4.12 (s, 2H), 3.72 (dd, J=16.0, 6.5 Hz, 1H), 3.43-3.36 (m, 1H), 3.20-2.97 (m, 3H), 2.87-2.75 (m, 1H), 2.33 (s, 3H), 2.24-1.96 (m, 3H), 1.93-1.74 (m, 3H), 1.56 (d, J=6.8 Hz, 3H). LC-MS: (ESI, m/z): 676.2 [M+H]⁺.

### Example 605: 4-(10-((R)-1-(2-Aminopyridin-3-yl)ethyl)-2-(((3R,5R)-2′,2′-difluoro-1-azaspiro[bicyclo[3.2.0]heptane-3,1′-cyclopropan]-5-yl)methoxy)-4-fluoro-9,10-dihydro-8H-cyclohepta[de]naphthalen-5-yl)-6-methyl-5-(trifluoromethyl)pyridin-2-amine

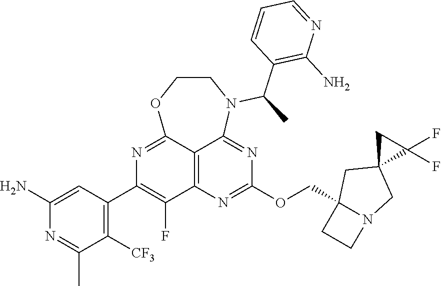 [SMILES: Cc1nc(N)cc(-c2nc3c4c(nc(OC[C@@]56CCN5C[C@]5(CC5(F)F)C6)nc4c2F)N([C@H](C)c2cccnc2N)CCO3)c1C(F)(F)F]

### Synthetic Route

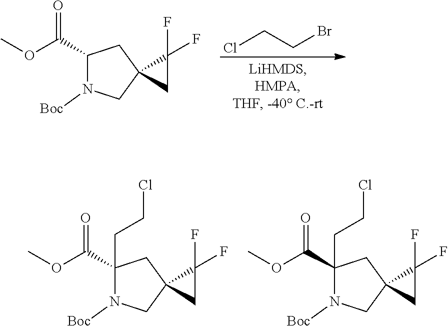

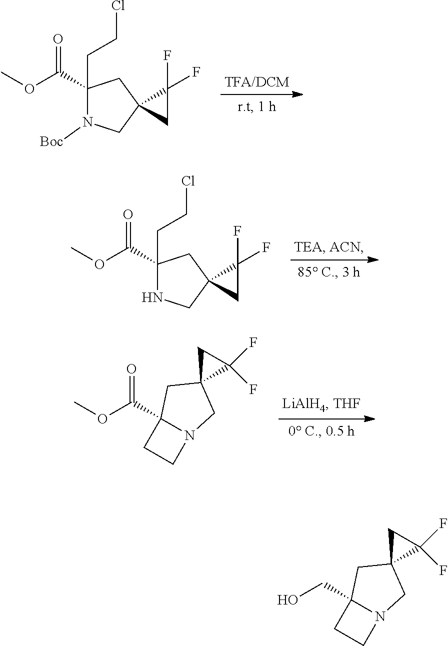

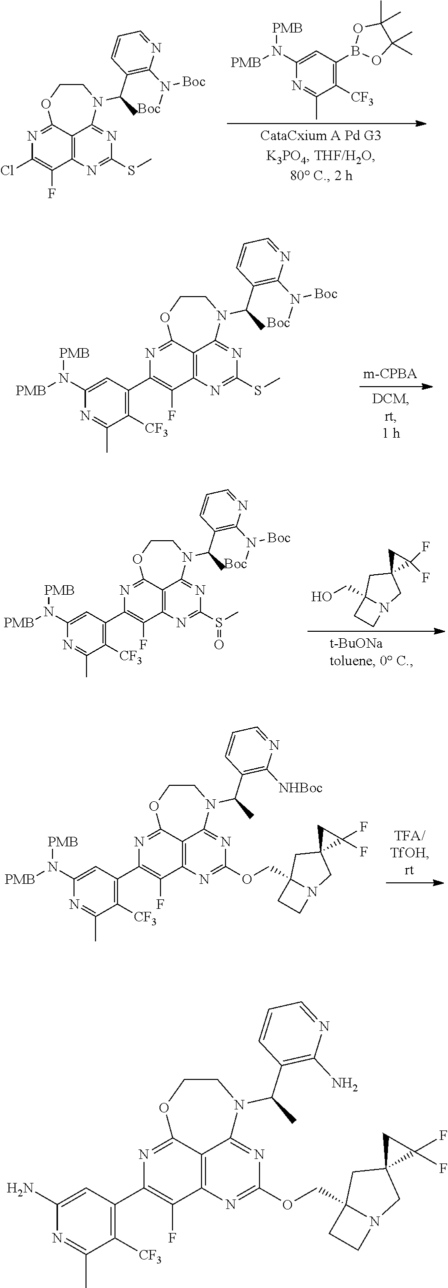

### Step 1: 5-(tert-butyl) 6-methyl (3R,6R)-6-(2-chloroethyl)-1,1-difluoro-5-azaspiro[2.4]heptane-5,6-dicarboxylate & 5-(tert-butyl) 6-methyl (3R,6S)-6-(2-chloroethyl)-1,1-difluoro-5-azaspiro[2.4]heptane-5,6-dicarboxylate

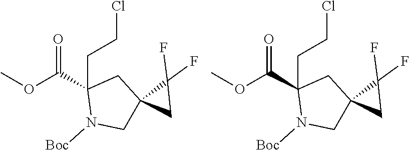

Under nitrogen, to a solution of 5-(tert-butyl) 6-methyl (3R,6S)-1,1-difluoro-5-azaspiro[2.4]heptane-5,6-dicarboxylate (500 mg, 1.72 mmol) and hexamethylphosphoramide (0.39 mL, 2.23 mmol) in tetrahydrofuran (7 mL) was added lithiumbis(trimethylsilyl)amide (2.0 mL, 2.00 mmol, 1M in tetrahydrofuran) at −40° C. The resulting solution was stirred for 0.5 h at −40° C. Then 1-bromo-2-chloroethane (0.7 mL, 8.58 mmol) was added at −40° C. and stirred at room temperature for 12 hours. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (97:3) to afford 5-(tert-butyl) 6-methyl (3R,6R)-6-(2-chloroethyl)-1,1-difluoro-5-azaspiro[2.4]heptane-5,6-dicarboxylate (250 mg, 0.71 mmol, 41% yield) as a light yellow oil and 5-(tert-butyl) 6-methyl (3R,6S)-6-(2-chloroethyl)-1,1-difluoro-5-azaspiro[2.4]heptane-5,6-dicarboxylate (150 mg, 0.42 mmol, 25% yield) as a light yellow oil. LC-MS: (ESI, m/z): LCMS (ESI, m/z): 298.1 [M+H-56]⁺.

### Step 2: methyl (3R,6R)-6-(2-chloroethyl)-1, 1-difluoro-5-azaspiro[2.4]heptane-6-carboxylate

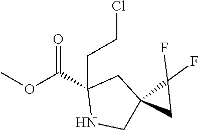 [SMILES: COC(=O)[C@]1(CCCl)C[C@]2(CN1)CC2(F)F]

A mixture of 5-(tert-butyl) 6-methyl (3R,6R)-6-(2-chloroethyl)-1,1-difluoro-5-azaspiro[2.4]heptane-5,6-dicarboxylate (1.0 g, 2.83 mmol) and trifluoroacetic acid (4.0 mL) in dichloromethane (15.0 mL) was stirred at room temperature for 1 hour. After completion, the reaction mixture was concentrated in vacuum and used in next step without further purification. LC-MS: (ESI, m/z): 253.8 [M+H]⁺

### Step 3: methyl (3R,5R)-2′,2′-difluoro-1-azaspiro[bicyclo[3.2.0]heptane-3,1′-cyclopropane]-5-carboxylate

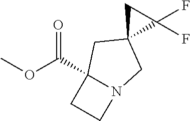

A mixture of methyl (3R,6R)-6-(2-chloroethyl)-1,1-difluoro-5-azaspiro[2.4]heptane-6-carboxylate (500 mg, 1.97 mmol) and triethylamine (1.4 mL, 9.86 mmol) in acetonitrile (10.0 mL) was stirred at 85° C. for 3 hours. The solvent was removed under vacuum. The residue was purified by flash chromatography on silica gel eluting with dichloromethane/methanol (96:4) to afford methyl (3R,5R)-2′,2′-difluoro-1-azaspiro[bicyclo[3.2.0]heptane-3,1′-cyclopropane]-5-carboxylate (300 mg, 1.38 mmol, 70% yield) as a yellow oil. LC-MS: (ESI, m/z): 218.1 [M+H]⁺

### Step 4: ((3R,5R)-2′,2′-difluoro-1-azaspiro[bicyclo[3.2.0]heptane-3,1′-cyclopropan]-5-yl)methanol

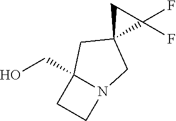 [SMILES: OC[C@@]12CCN1C[C@]1(CC1(F)F)C2]

To a solution of methyl (3S,5R)-1′,1′-difluorospiro[1-azabicyclo[3.2.0]heptane-3,2′-cyclopropane]-5-carboxylate (300 mg, 1.38 mmol) in tetrahydrofuran (5.0 mL) was added lithium aluminum hydride (157 mg, 4.14 mmol), the mixture was stirred for 0.5 hours at 0° C. After completion, the reaction was quenched with sodium sulfate decahydrate. The solids were filtered out and washed with tetrahydrofuran. After filtration, the filtrate was concentrated under reduced pressure. The crude would be directly used in the next step without further purification. LC-MS: (ESI, m/z): 190.2 [M+H]⁺

### Step 5: tert-butyl N-[3-[(1R)-1-[7-[6-[bis[(4-methoxyphenyl)methyl]amino]-2-methyl-3-(trifluoromethyl)-4-pyridyl]-6-fluoro-3-methylsulfanyl-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate

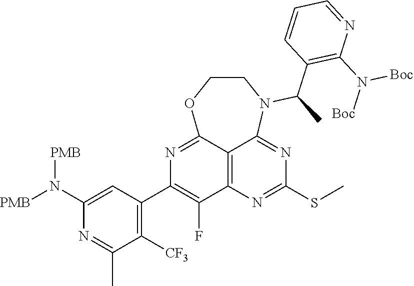

Under nitrogen, a mixture of tert-butyl N-tert-butoxycarbonyl-N-[3-[(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl]-2-pyridyl]carbamate (300 mg, 0.49 mmol), N,N-bis[(4-methoxyphenyl)methyl]-6-methyl-4-(4,4,5,5-tetramethyl-1,3,2-dioxaborolan-2-yl)-5-(trifluoromethyl)pyridin-2-amine (402 mg, 0.74 mmol), [(di(1-adamantyl)-butylphosphine)-2-(2′-amino-1,1′-biphenyl)]palladium(II) methanesulfonate (36 mg, 0.05 mmol) and potassium phosphate (315 mg, 1.48 mmol) in tetrahydrofuran (5.0 mL) and water (1.0 mL) was stirred at 80° C. for 2 hours. After completion, the reaction mixture was diluted with water and extracted with dichloromethane. The combined organic layers were washed with brine, dried over anhydrous sodium sulfate and concentrated in vacuum. The residue was purified by flash chromatography on silica gel eluting with petroleum ether/ethyl acetate (2:1) to afford tert-butyl N-[3-[(1R)-1-[7-[6-[bis[(4-methoxyphenyl)methyl]amino]-2-methyl-3-(trifluoromethyl)-4-pyridyl]-6-fluoro-3-methylsulfanyl-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate as a light yellow solid (510 mg, 63% purity, 0.32 mmol, 66% yield). LC-MS: (ESI, m/z): 987.2 [M+H]⁺

### Step 6: tert-butyl N-[3-[(1R)-1-[7-[6-[bis[(4-methoxyphenyl)methyl]amino]-2-methyl-3-(trifluoromethyl)-4-pyridyl]-6-fluoro-3-methylsulfinyl-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate

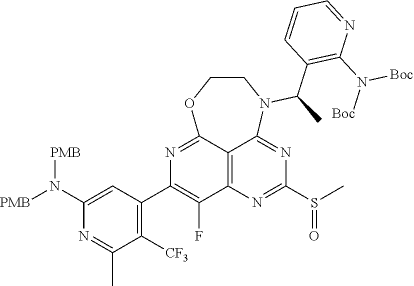

A mixture of tert-butyl N-[3-[(1R)-1-[7-[6-[bis[(4-methoxyphenyl)methyl]amino]-2-methyl-3-(trifluoromethyl)-4-pyridyl]-6-fluoro-3-methylsulfanyl-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (380 mg, 0.38 mmol) and 3-chloroperoxybenzoic acid (117 mg, 0.58 mmol) in dichloromethane (8.0 mL) was stirred at room temperature for 1 hour. After completion, the reaction mixture was quenched with saturated sodium sulfite solution and extracted with dichloromethane. The combined organic layers were washed with brine, dried over anhydrous sodium sulfate and concentrated in vacuum. The residue was purified by flash chromatography on silica gel eluting with petroleum ether/ethyl acetate (2:1) to afford tert-butyl N-[3-[(1R)-1-[7-[6-[bis[(4-methoxyphenyl)methyl]amino]-2-methyl-3-(trifluoromethyl)-4-pyridyl]-6-fluoro-3-methylsulfinyl-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate as a light yellow oil (350 mg, 76% purity, 0.26 mmol, 70% yield). LC-MS: (ESI, m/z): 1003.4 [M+H]⁺

### Step 7: tert-butyl (3-((R)-1-(5-(6-(bis(4-methoxybenzyl)amino)-2-methyl-3-(trifluoromethyl)pyridin-4-yl)-2-(((3R,5R)-2′,2′-difluoro-1-azaspiro[bicyclo[3.2.0]heptane-3,1′-cyclopropan]-5-yl)methoxy)-4-fluoro-8,9-dihydro-10H-cyclohepta[de]naphthalen-10-yl)ethyl)pyridin-2-yl)carbamate

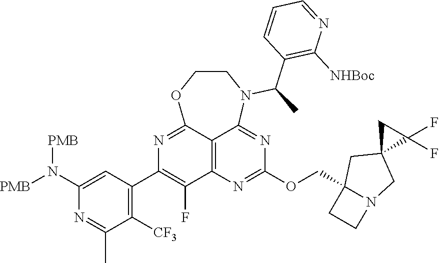

Sodium tert-butoxide (158 mg, 1.64 mmol) was added to a solution of [(3R,5R)-1′,1′-difluorospiro[1-azabicyclo[3.2.0]heptane-3,2′-cyclopropane]-5-yl]methanol (187 mg, 0.99 mmol) in dry toluene (4.0 mL) under nitrogen atmosphere at 0° C., and the mixture was stirred at 0° C. for 20 min. Then the mixture was transferred to a solution of tert-butyl N-[3-[(1R)-1-[7-[6-[bis[(4-methoxyphenyl)methyl]amino]-2-methyl-3-(trifluoromethyl)-4-pyridyl]-6-fluoro-3-methylsulfinyl-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (330 mg, 0.33 mmol) in toluene (4.0 mL) and stirred at 0° C. for 10 min. After completion, the reaction was quenched with saturated ammonium chloride solution. The resulting solution was diluted with water and extracted with ethyl acetate. The organic layers were combined, washed with brine, dried over anhydrous sodium sulfate and concentrated. The resulting residue was purified by reverse phase chromatography (acetonitrile 80-85/0.1% NH₄HCO₃ in water) to afford tert-butyl (3-((R)-1-(5-(6-(bis(4-methoxybenzyl)amino)-2-methyl-3-(trifluoromethyl)pyridin-4-yl)-2-(((3R,5R)-2′,2′-difluoro-1-azaspiro[bicyclo[3.2.0]heptane-3,1′-cyclopropan]-5-yl)methoxy)-4-fluoro-8,9-dihydro-10H-cyclohepta[de]naphthalen-10-yl)ethyl)pyridin-2-yl)carbamate as a light yellow solid (150 mg, 91% purity, 0.15 mmol, 44% yield). LC-MS: (ESI, m/z): 1028.0 [M+H]⁺

### Step 8: 4-(10-((R)-1-(2-Aminopyridin-3-yl)ethyl)-2-(((3R,5R)-2′,2′-difluoro-1-azaspiro[bicyclo[3.2.0]heptane-3,1′-cyclopropan]-5-yl)methoxy)-4-fluoro-9,10-dihydro-8H-cyclohepta[de]naphthalen-5-yl)-6-methyl-5-(trifluoromethyl)pyridin-2-amine

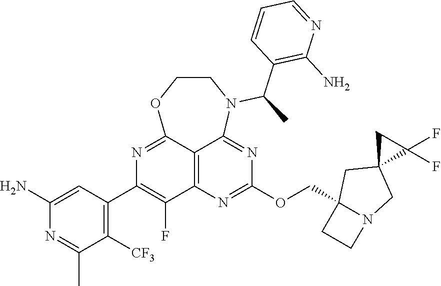 [SMILES: Cc1nc(N)cc(-c2nc3c4c(nc(OC[C@@]56CCN5C[C@]5(CC5(F)F)C6)nc4c2F)N([C@H](C)c2cccnc2N)CCO3)c1C(F)(F)F]

A solution of tert-butyl (3-((R)-1-(5-(6-(bis(4-methoxybenzyl)amino)-2-methyl-3-(trifluoromethyl)pyridin-4-yl)-2-(((3R,5R)-2′,2′-difluoro-1-azaspiro[bicyclo[3.2.0]heptane-3,1′-cyclopropan]-5-yl)methoxy)-4-fluoro-8,9-dihydro-10H-cyclohepta[de]naphthalen-10-yl)ethyl)pyridin-2-yl)carbamate (135 mg, 0.13 mmol) in trifluoroacetic acid (4.0 mL) and trifluoromethanesulfonic acid (0.4 mL) was stirred at room temperature for 10 min. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: (Column: YMC Triart C18 ExRs Sum, 30*150 mm; Mobile Phase A: Water(10 mmol/L NH₄HCO₃), Mobile Phase B: ACN; Flow rate: 60 mL/min; Gradient: 32% B to 48% B in 10 min; Wave Length: 254 nm/220 nm) to afford 4-(10-((R)-1-(2-Aminopyridin-3-yl)ethyl)-2-(((3R,5R)-2′,2′-difluoro-1-azaspiro[bicyclo[3.2.0]heptane-3,1′-cyclopropan]-5-yl)methoxy)-4-fluoro-9,10-dihydro-8H-cyclohepta[de]naphthalen-5-yl)-6-methyl-5-(trifluoromethyl)pyridin-2-amine (40.7 mg, 0.059 mmol, 46% yield, 98.6% purity) as a white solid.

Example 605: ¹H NMR (400 MHz, Methanol-d₄, ppm) δ 8.05 (d, J=7.4 Hz, 1H), 7.97 (dd, J=5.9, 1.6 Hz, 1H), 6.96 (dd, J=7.5, 5.8 Hz, 1H), 6.62 (q, J=6.8 Hz, 1H), 6.35 (s, 1H), 5.03 (d, J=12.8 Hz, 1H), 4.78 (d, J=12.8 Hz, 1H), 4.64-4.48 (m, 2H), 4.43 (dd, J=13.1, 6.6 Hz, 1H), 4.09-3.94 (m, 1H), 3.95-3.82 (m, 2H), 3.63 (dd, J=16.0, 6.4 Hz, 1H), 3.49 (d, J=12.7 Hz, 1H), 2.95-2.79 (m, 1H), 2.79-2.66 (m, 2H), 2.54 (d, J=2.2 Hz, 3H), 2.30 (d, J=14.0 Hz, 1H), 2.16-1.96 (m, 2H), 1.71 (d, J=6.8 Hz, 3H). LC-MS: (ESI, m/z): 688.3 [M+H]⁺

### Example 606: 5-((R)-1-(5-(6-amino-2-methyl-3-(trifluoromethyl)pyridin-4-yl)-2-(((3R,5R)-2′,2′-difluoro-1-azaspiro[bicyclo[3.2.0]heptane-3,1′-cyclopropan]-5-yl)methoxy)-4-fluoro-8,9-dihydro-10H-cyclohepta[de]naphthalen-10-yl)ethyl)pyrimidin-4-amine

### Synthetic Route

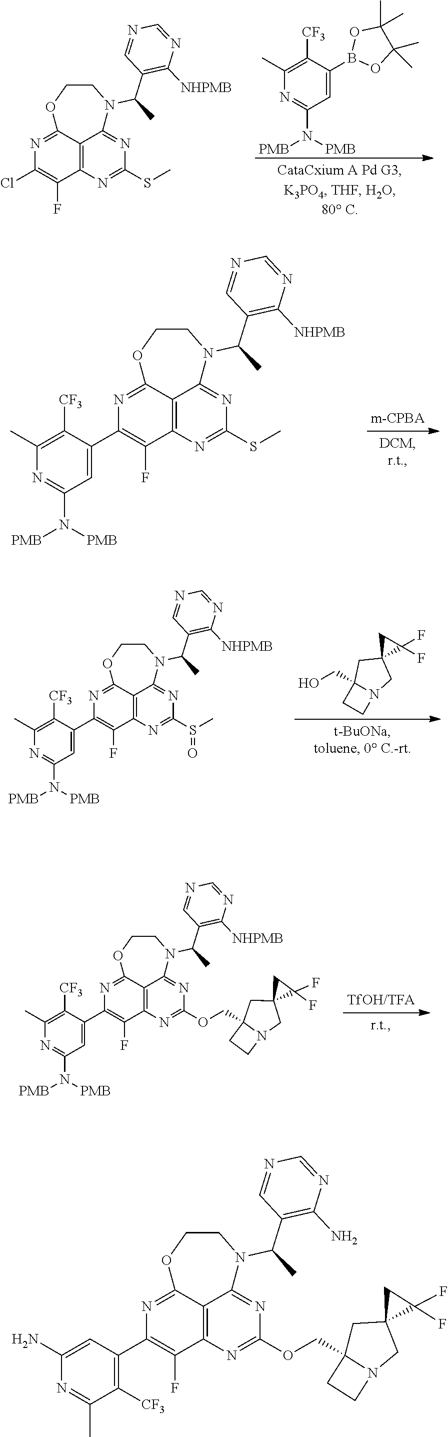

### Step 1: (R)-5-(1-(5-(6-(bis(4-methoxybenzyl)amino)-2-methyl-3-(trifluoromethyl)pyridin-4-yl)-4-fluoro-2-(methylthio)-8,9-dihydro-10H-7-oxa-13,6,10-tetraazacyclohepta[de]naphthalen-10-yl)ethyl)-N-(4-methoxybenzyl)pyrimidin-4-amine

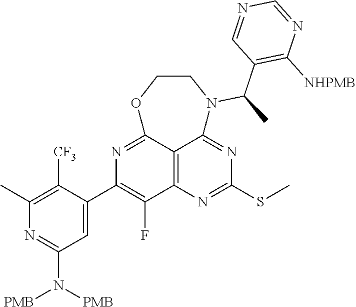

Under nitrogen, a solution of (R)-5-(1-(5-chloro-4-fluoro-2-(methylthio)-8,9-dihydro-1 OH-7-oxa-1,3,6,10-tetraazacyclohepta[de]naphtha len-10-yl)ethyl)-N-(4-methoxybenzyl)pyrimidin-4-amine (100.0 mg, 0.19 mmol), N,N-bis(4-methoxybenzyl)-6-methyl-4-(4,4,5,5-tetramethyl-1,3,2-dioxaborolan-2-yl)-5-(trifIuoromethyl) pyridin-2-amine (256.8 mg, 0.47 mmol), potassium phosphate (120.6 mg, 0.57 mmol) and methanesulfonato(diadamantyl-n-butylphosphino)-2′-amino-1,1′-biphenyl-2-yl)palladium(II) dichloromethane adduct (13.7 mg, 0.02 mmol) in tetrahydrofuran (1 mL) and water (0.20 mL) was stirred at 80° C. for 2 hours. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (1:1) to afford (R)-5-(1-(5-(6-(bis(4-methoxybenzyl)amino)-2-methyl-3-(trifluoromethyl)pyridin-4-yl)-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrimidin-4-amine (150.0 mg, 0.16 mmol, 87.2% yield) as a yellow solid. LC-MS: (ESI, m/z): 908.3 [M+H]⁺.

### Step 2: 5-((1R)-1-(5-(6-(bis(4-methoxybenzyl)amino)-2-methyl-3-(trifluoromethyl)pyridin-4-yl)-4-fluoro-2-(methylsulfinyl)-8,9-dihydro-10H-7-oxa-1,3,6,10-tetraazacyclohepta[de]naphthalen-10-yl)ethyl)-N-(4-methoxybenzyl)pyrimidin-4-amine

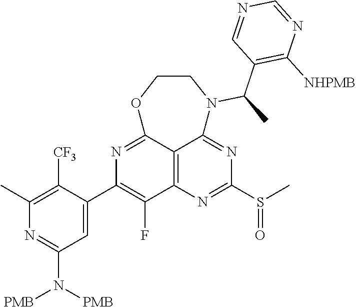

To a solution of (R)-5-(1-(5-(6-(bis(4-methoxybenzyl)amino)-2-methyl-3-(trifluoromethyl)pyridin-4-yl)-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrimidin-4-amine (140.0 mg, 0.15 mmol) in dichloromethane (2 mL) was added 3-chloroperoxybenzoic acid (66.5 mg, 0.39 mmol), the mixture was stirred at 25° C. for 0.5 hours. After completion, the reaction was quenched with saturated sodium thiosulfate solution. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1:1) to afford 5-((1R)-1-(5-(6-(bis(4-methoxybenzyl)amino)-2-methyl-3-(trifluoromethyl)pyridin-4-yl)-4-fluoro-2-(methylsulfinyl)-8,9-dihydro-10H-7-oxa-1,3,6,10-tetraazacyclohepta[de]naphthalen-10-yl)ethyl)-N-(4-methoxybenzyl)pyrimidin-4-amine (100.0 mg, 0.09 mmol, 59.2% yield) as a yellow solid. LC-MS: (ESI, m/z): 924.3 [M+H]⁺.

### Step 3: 5-((R)-1-(5-(6-(bis(4-methoxybenzyl)amino)-2-methyl-3-(trifluoromethyl)pyridin-4-yl)-2-(((3R,5R)-2′,2′-difluoro-1-azaspiro[bicyclo[3.2.0]heptane-3,1′-cyclopropan]-5-yl)methoxy)-4-fluoro-8,9-dihydro-10H-cyclohepta[de]naphthalen-10-yl)ethyl)-N-(4-methoxybenzyl)pyrimidin-4-amine

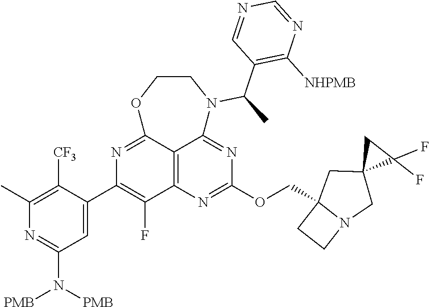

To a solution of ((3R,5R)-2′,2′-difluoro-1-azaspiro[bicyclo[3.2.0]heptane-3,1′-cyclopropan]-5-yl)methanol (49.1 mg, 0.26 mmol) in toluene (1 mL) was added sodium tert-butoxide (24.9 mg, 0.26 mmol), the mixture was stirred for 10 minutes at 0° C. Then 5-((1R)-1-(5-(6-(bis(4-methoxybenzyl)amino)-2-methyl-3-(trifluoromethyl)pyridin-4-yl)-4-fluoro-2-(methylsulfinyl)-8,9-dihydro-10H-7-oxa-1,3,6,10-tetraazacyclohepta[de]naphthalen-10-yl)ethyl)-N-(4-methoxybenzyl)pyrimidin-4-amine (80.0 mg, 0.09 mmol) in toluene (0.2 mL) was added and stirred at 0° C. for 0.5 hours. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1:1) to afford 5-((R)-1-(5-(6-(bis(4-methoxybenzyl)amino)-2-methyl-3-(trifluoromethyl)pyridin-4-yl)-2-(((3R,5R)-2′,2′-difluoro-1-azaspiro[bicyclo[3.2.0]heptane-3,1′-cyclopropan]-5-yl)methoxy)-4-fluoro-8,9-dihydro-10H-cyclohepta[de]naphthalen-10-yl)ethyl)-N-(4-methoxybenzyl)pyrimidin-4-amine (35.0 mg, 0.03 mmol, 38.5% yield) as a yellow solid. LC-MS: (ESI, m/z): 1049.4 [M+H]⁺.

### Step 4: 5-((R)-1-(5-(6-amino-2-methyl-3-(trifluoromethyl)pyridin-4-yl)-2-(((3R,5R)-2′,2′-difluoro-1-azaspiro[bicyclo[3.2.0]heptane-3,1′-cyclopropan]-5-yl)methoxy)-4-fluoro-8,9-dihydro-10H-cyclohepta[de]naphthalen-10-yl)ethyl)pyrimidin-4-amine

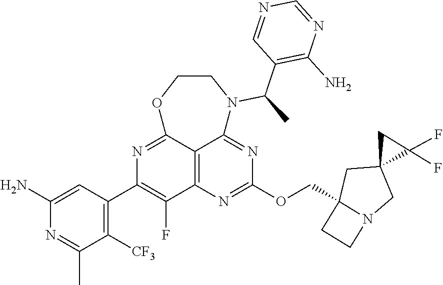 [SMILES: Cc1nc(N)cc(-c2nc3c4c(nc(OC[C@@]56CCN5C[C@]5(CC5(F)F)C6)nc4c2F)N([C@H](C)c2cncnc2N)CCO3)c1C(F)(F)F]

A solution of 5-((R)-1-(5-(6-(bis(4-methoxybenzyl)amino)-2-methyl-3-(trifluoromethyl)pyridin-4-yl)-2-(((3R,5R)-2′,2′-difluoro-1-azaspiro[bicyclo[3.2.0]heptane-3,1′-cyclopropan]-5-yl)methoxy)-4-fluoro-8,9-dihydro-10H-cyclohepta[de]naphthalen-10-yl)ethyl)-N-(4-methoxybenzyl)pyrimidin-4-amine (30.0 mg, 0.03 mmol) in trifluoromethanesulfonic acid (0.1 mL) and 2,2,2-trifluoroacetic acid (1 mL) was stirred at 25° C. for 10 hours. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (8:1) to afford crude product. The crude product was purified by Prep-HPLC with the following conditions: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 17% B to 42% B in 9 min; Wave Length: 254 nm/220 nm to afford 5-((R)-1-(5-(6-amino-2-methyl-3-(trifluoromethyl)pyridin-4-yl)-2-(((3R,5R)-2′,2′-difluoro-1-azaspiro[bicyclo[3.2.0]heptane-3,1′-cyclopropan]-5-yl)methoxy)-4-fluoro-8,9-dihydro-10H-cyclohepta[de]naphthalen-10-yl)ethyl)pyrimidin-4-amine (6.2 mg, 0.01 mmol, 31.1% yield) as a white solid. LC-MS: (ESI, m/z): 689.2[M+H]⁺.

Example 606: ¹H NMR (300 MHz, Methanol-d₄, ppm) δ 8.39 (s, 1H), 8.32 (s, 1H), 6.58 (q, J=6.9 Hz, 1H), 6.33 (s, 1H), 4.63-4.28 (m, 4H), 3.84 (dd, J=16.0, 6.5 Hz, 1H), 3.75-3.51 (m, 2H), 3.49-3.34 (m, 1H), 3.29-3.24 (m, 1H), 2.77 (d, J=12.8 Hz, 1H), 2.67-2.57 (m, 1H), 2.57-2.43 (m, 4H), 2.42-2.27 (m, 1H), 2.21-2.06 (m, 1H), 1.80-1.58 (m, 5H). LC-MS: (ESI, m/z): 689.2[M+H]⁺.

### Example 607: 5-(1-(5-(6-amino-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)thiazol-4-amine

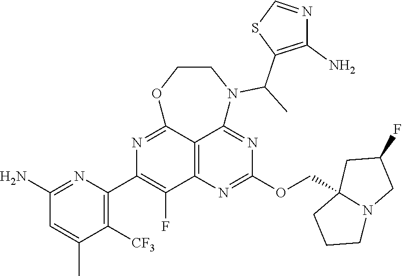 [SMILES: Cc1cc(N)nc(-c2nc3c4c(nc(OC[C@@]56CCCN5C[C@H](F)C6)nc4c2F)N(C(C)c2scnc2N)CCO3)c1C(F)(F)F]

### Synthetic Route

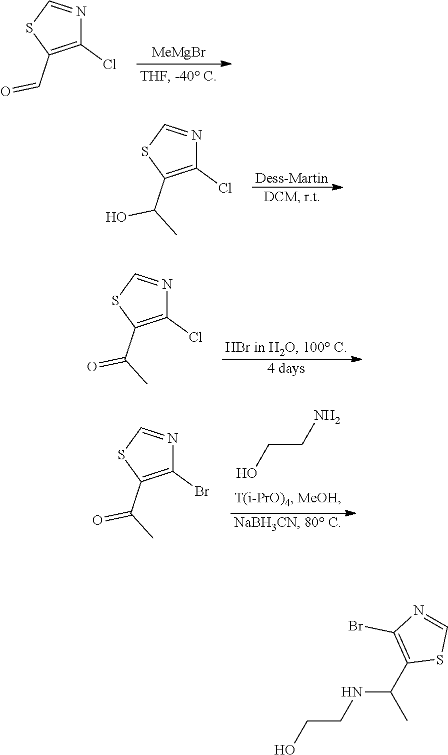

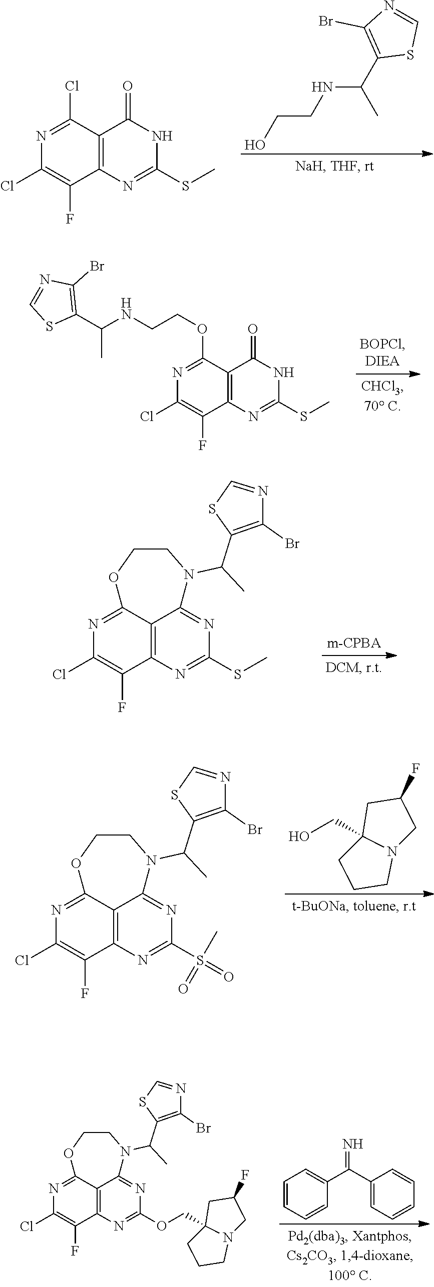

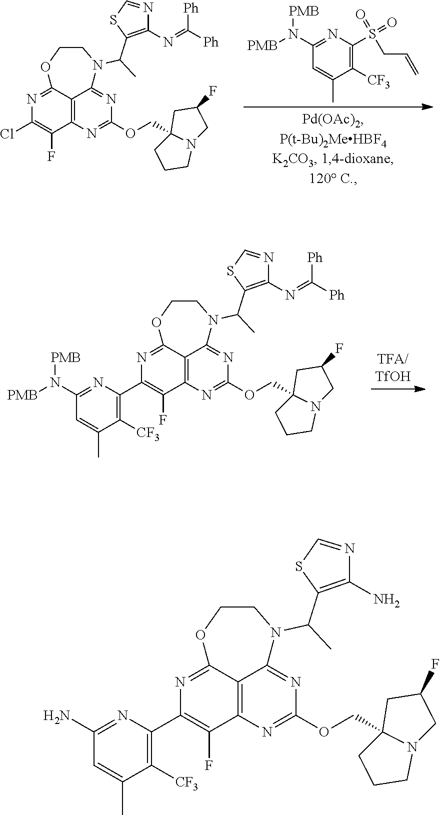

### Step 1: 1-(4-chlorothiazol-5-yl)ethan-1-ol

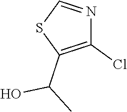

To a solution of 4-chlorothiazole-5-carbaldehyde (15.0 g, 96.90 mmol) in tetrahydrofuran (143.00 mL) was added methylmagnesiumbromide (193.70 mL, 193.70 mmol, 1 mol/Lin tetrahydrofuran) at −40° C., the mixture was stirred at −40° C. for 1 hour. After completion, the reaction was quenched with 1 M hydrochloric acid. The organic layer was concentrated in vacuum. The residue was purified by flash chromatography on silica gel eluting with petroleum ether/ethyl acetate (3:1) to afford 1-(4-chlorothiazol-5-yl)ethan-1-ol (14.20 g, 75.40 mmol, 77.9% yield) as a yellow liquid. LC-MS: (ESI, m/z): 164.0 [M+H]⁺

### Step 2: 1-(4-chlorothiazol-5-yl)ethan-1-one

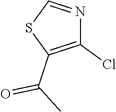

To a solution of 1-(4-chlorothiazol-5-yl)ethan-1-ol (14.20 g, 86.40 mmol) in dichloromethane (500 mL) was added Dess-Martin periodinane (54.18 g, 129.84 mmol), the mixture was stirred for 3 hours at 25° C. The reaction was quenched with calcium hydroxide. The solids were filtered out. After filtration, the filtrate was concentrated under reduced pressure. The organic layer was concentrated in vacuum. The residue was purified by flash chromatography on silica gel eluting with petroleum ether/ethyl acetate (93:7) to afford 1-(4-chlorothiazol-5-yl)ethan-1-one (12.60 g, 78.02 mmol, 89.3% yield) as a white solid. LC-MS: (ESI, m/z): 162.0 [M+H]⁺

### Step 3: 1-(4-bromothiazol-5-yl)ethan-1-one

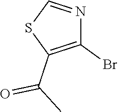

A solution of 1-(4-chlorothiazol-5-yl)ethan-1-one (12.50 g, 74.20 mmol) in hydrogen bromide (119 mL, 48%) was stirred at 100° C. for 4 days. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with petroleum ether/ethyl acetate (93:7) to afford 1-(4-bromothiazol-5-yl)ethan-1-one (7.00 g, 27.72 mmol, 49.2% yield) as a yellow solid. LC-MS: (ESI, m/z): 205.9[M+H]⁺

### Step 4: 2-((1-(4-bromothiazol-5-yl)ethyl)amino)ethan-1-ol

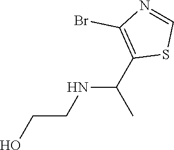

A mixture of 1-(4-bromothiazol-5-yl)ethan-1-one (6.90 g, 33.49 mmol), 2-aminoethanol (4.09 g, 66.97 mmol), titanium(iv)isopropoxide (9.52 g, 33.49 mmol) and sodium cyanoborohydride (6.31 g, 100.46 mmol) in methanol (30 mL) was stirred at 80° C. for 2 hours. After completion, the reaction was quenched with water. The resulting solution was extracted with ethyl acetate. The organic layers were washed with brine, dried over anhydrous sodium sulfate and concentrated. The residue was purified by flash chromatography on silica gel eluting with dichloromethane/methanol (96:4) to afford 2-((1-(4-bromothiazol-5-yl)ethyl)amino)ethan-1-ol (7.00 g, 27.87 mmol, 83.2% yield) as a yellow solid. LC-MS: (ESI, m/z): 251.0 [M+H]⁺

### Step 5: 5-(2-((1-(4-bromothiazol-5-yl)ethyl)amino)ethoxy)-7-chloro-8-fluoro-2-(methylthio)pyrido[4,3-d]pyrimidin-4(3H)-one

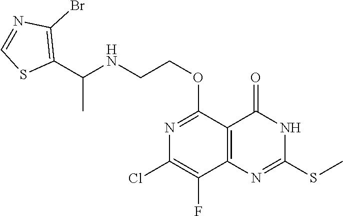 [SMILES: CSc1nc2c(F)c(Cl)nc(OCCNC(C)c3scnc3Br)c2c(=O)[nH]1]

To a solution of 2-((1-(4-bromothiazol-5-yl)ethyl)amino)ethan-1-ol (7.00 g, 27.45 mmol) in tetrahydrofuran (60 mL) was added sodium hydride (28.8 g, 0.72 mmol, 60% dispersion in mineral oil), the mixture was stirred for 10 minutes at 0° C. Then the mixture was transferred into a solution of 5,7-dichloro-8-fluoro-2-methylsulfanyl-3H-pyrido[4,3-d]pyrimidin-4-one (5.50 g, 19.64 mmol) in tetrahydrofuran (30 mL), stirred for 2 hours at room temperature. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (2:8) to afford 5-(2-((1-(4-bromothiazol-5-yl)ethyl)amino)ethoxy)-7-chloro-8-fluoro-2-(methylthio)pyrido[4,3-d]pyrimidin-4(3H)-one (8.2 g, 16.57 mmol, 84.4% yield) as a yellow solid. LC-MS: (ESI, m/z): 493.9 [M+H]⁺

### Step 6: 10-(1-(4-bromothiazol-5-yl)ethyl)-5-chloro-4-fluoro-2-(methylthio)-9,10-dihydro-8H-7-oxa-1,3,6,10-tetraazacyclohepta[de]naphthalene

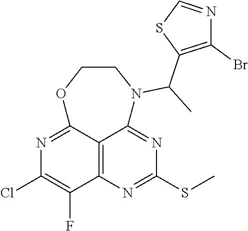 [SMILES: CSc1nc2c3c(nc(Cl)c(F)c3n1)OCCN2C(C)c1scnc1Br]

A mixture of 5-(2-((1-(4-bromothiazol-5-yl)ethyl)amino)ethoxy)-7-chloro-8-fluoro-2-(methylthio)pyrido[4,3-d]pyrimidin-4(3H)-one (8.0 g, 16.17 mmol), bis(2-oxo-3-oxazolidinyl)phosphinicchloride (6.17 g, 24.25 mmol) and N,N-diisopropylethylamine (14.08 mL, 80.84 mmol) in chloroform (80 mL) was stirred at 70° C. for 1 hour. The resulting solution was diluted with water and extracted with dichloromethane. The organic layers were washed with brine, dried over anhydrous sodium sulfate and concentrated. The residue was purified by flash chromatography on silica gel eluting with petroleum ether/ethyl acetate (1:1) to afford 10-(1-(4-bromothiazol-5-yl)ethyl)-5-chloro-4-fluoro-2-(methylthio)-9,10-dihydro-8H-7-oxa-1,3,6,10-tetraazacyclohepta[de]naphthalene (4.70 g, 9.85 mmol, 61% yield) as a yellow solid. LC-MS: (ESI, m/z): 475.9 [M+H]⁺

### Step 7: 10-(1-(4-bromothiazol-5-yl)ethyl)-5-chloro-4-fluoro-2-(methylsulfonyl)-9,10-dihydro-8H-7-oxa-1,3,6,10-tetraazacyclohepta[de]naphthalene

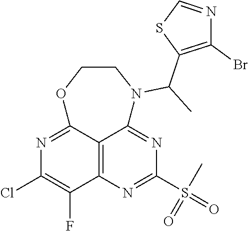 [SMILES: CC(c1scnc1Br)N1CCOc2nc(Cl)c(F)c3nc(S(C)(=O)=O)nc1c23]

To a mixture of 10-(1-(4-bromothiazol-5-yl)ethyl)-5-chloro-4-fluoro-2-(methylthio)-9,10-dihydro-8H-7-oxa-1,3,6,10-tetraazacyclohepta[de]naphthalene (4.20 g, 8.81 mmol) in dichloromethane (15 mL) was added 3-chloroperoxybenzoic acid (3.80 g, 22.02 mmol), the mixture was stirred for 30 minutes at room temperature. After completion, the reaction was quenched with saturated sodium sulfite solution. The resulting solution was extracted with ethyl acetate. The organic layers were washed with brine, dried over anhydrous sodium sulfate and concentrated. The residue was purified by flash chromatography on silica gel eluting with petroleum ether/ethyl acetate (1:2) to afford 10-(1-(4-bromothiazol-5-yl)ethyl)-5-chloro-4-fluoro-2-(methylsulfonyl)-9,10-dihydro-8H-7-oxa-1,3,6,10-tetraazacyclohepta[de]naphthalene (3.70 g, 7.27 mmol, 82.6% yield) as a white solid. LC-MS: (ESI, m/z): 507.9 [M+H]⁺

### Step 8: 10-(1-(4-bromothiazol-5-yl)ethyl)-5-chloro-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalene

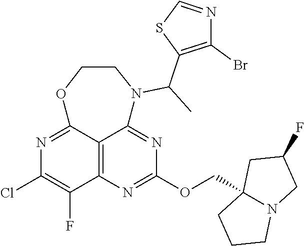 [SMILES: CC(c1scnc1Br)N1CCOc2nc(Cl)c(F)c3nc(OC[C@@]45CCCN4C[C@H](F)C5)nc1c23]

To a solution of ((2R,7aS)-2-fluorotetrahydro-1H-pyrrolizin-7a(5H)-yl)methanol (1.35 g, 8.49 mmol) in toluene (9 mL) was added sodium tert-butoxide (2.04 g, 21.23 mmol), the mixture was stirred at 0° C. for 10 minutes, then 10-(1-(4-bromothiazol-5-yl)ethyl)-5-chloro-4-fluoro-2-(methylsulfonyl)-9,10-dihydro-8H-7-oxa-1,3,6,10-tetraazacyclohepta[de]naphthalene (3.60 g, 7.08 mmol) in toluene (5 mL) was added and stirred at 0° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (98:2) to afford 10-(1-(4-bromothiazol-5-yl)ethyl)-5-chloro-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalene (610.0 mg, 0.87 mmol, 10.6% yield) as a yellow solid. LC-MS: (ESI, m/z): 587.0 [M+H]⁺

### Step 9: N-(5-(1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)thiazol-4-yl)-1,1-diphenylmethanimine

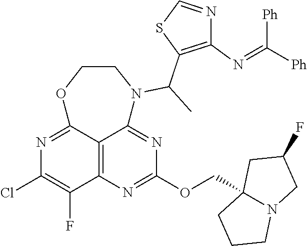 [SMILES: CC(c1scnc1N=C(c1ccccc1)c1ccccc1)N1CCOc2nc(Cl)c(F)c3nc(OC[C@@]45CCCN4C[C@H](F)C5)nc1c23]

Under nitrogen, a mixture of 10-(1-(4-bromothiazol-5-yl)ethyl)-5-chloro-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalene (600.0 mg, 1.02 mmol), benzophenone imine (0.19 mL, 1.12 mmol), tris(dibenzylideneacetone)dipalladium-chloroform adduct (93.5 mg, 0.10 mmol), 4,5-bis(diphenylphosphino)-9,9-dimethylxanthene (118.1 mg, 0.20 mmol) and cesium carbonate (1003.8 mg, 3.06 mmol) in 1,4-dioxane (6 mL) was stirred at 100° C. for 5 hours. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (68:32) to afford N-(5-(1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)thiazol-4-yl)-1,1-diphenylmethanimine (130.0 mg, 0.12 mmol, 11.8% yield) as a yellow solid. LC-MS: (ESI, m/z): 688.2 [M+H]⁺

### Step 10: 6-(10-(1-(4-((diphenylmethylene)amino)thiazol-5-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-4-methyl-5-(trifluoromethyl)pyridin-2-amine

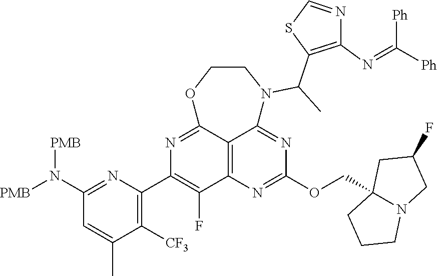

A solution of N-(5-(1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)thiazol-4-yl)-1,1-diphenylmethanimine (130.0 mg, 0.19 mmol), 6-allylsulfonyl-N,N-bis[(4-methoxyphenyl)methyl]-4-methyl-5-(trifluoromethyl)pyridin-2-amine (147.5 mg, 0.28 mmol), palladium(II)acetate (4.28 mg, 0.02 mmol), di-tert-butylmethylphosphonium tetrafluoroborate (9.37 mg, 0.04 mmol) and potassium carbonate (78.32 mg, 0.57 mmol) in 1,4-dioxane (1 mL) was stirred at 120° C. for 1 hour. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (20:80) to afford 6-(10-(1-(4-((diphenylmethylene)amino)thiazol-5-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-4-methyl-5-(trifluoromethyl)pyridin-2-amine (100 mg, 0.06 mmol, 30.2% yield) as a yellow solid. LC-MS: (ESI, m/z): 1068.4 [M+H]⁺

### Step 11: 5-(1-(5-(6-amino-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)thiazol-4-amine

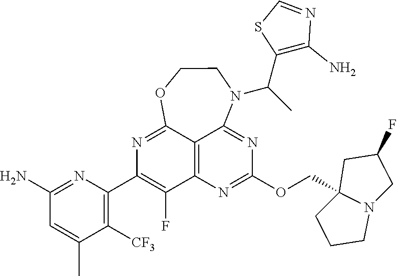 [SMILES: Cc1cc(N)nc(-c2nc3c4c(nc(OC[C@@]56CCCN5C[C@H](F)C6)nc4c2F)N(C(C)c2scnc2N)CCO3)c1C(F)(F)F]

A mixture of 6-(10-(1-(4-((diphenylmethylene)amino)thiazol-5-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-4-methyl-5-(trifluoromethyl)pyridin-2-amine (100.0 mg, 0.09 mmol) in trifluoromethanesulfonic acid (0.1 mL) and 2,2,2-trifluoroacetic acid (1 mL) was stirred at room temperature for 30 minutes. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (85:15) to afford crude solid. The crude product was purified by Prep-HPLC with the following conditions: Column: YMC Triart C18 ExRs, 30*150 mm, 5 μm; Mobile Phase A: Water(10 mmol/L NH₄HCO₃), Mobile Phase B: ACN; Flow rate: 60 mL/min; Gradient: 33% B to 55% B in 10 min; Wave Length: 254 nm/220 nm to afford 5-(1-(5-(6-amino-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)thiazol-4-amine (13.8 mg, 0.02 mmol, 22.1% yield) as a white solid. LC-MS: (ESI, m/z): 664.4[M+H]⁺

Example 607: ¹H NMR (300 MHz, DMSO-d₆, ppm) δ 8.74 (s, 1H), 6.78 (s, 2H), 6.47 (s, 2H), 5.52-5.17 (m, 3H), 4.49 (dd, J=12.8, 5.9 Hz, 1H), 4.36 (dd, J=12.7, 6.4 Hz, 1H), 4.28-4.00 (m, 2H), 3.89-3.71 (m, 1H), 3.63-3.49 (m, 1H), 3.20-3.00 (m, 3H), 2.94-2.79 (m, 1H), 2.36 (s, 3H), 2.22-1.93 (m, 3H), 1.91-1.73 (m, 3H), 1.59 (d, J=6.9 Hz, 3H). LC-MS: (ESI, m/z): 664.4[M+H]⁺

### Example 608: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-(trifluoromethyl)pyridin-2-amine

### Synthetic Route

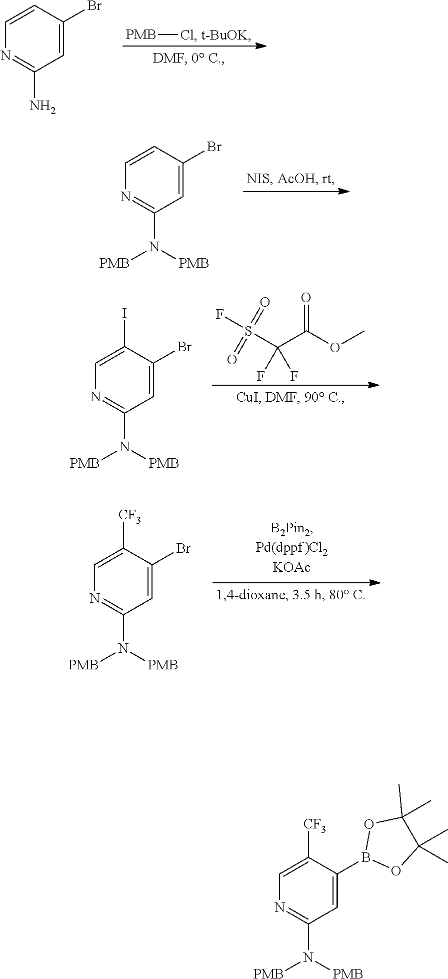

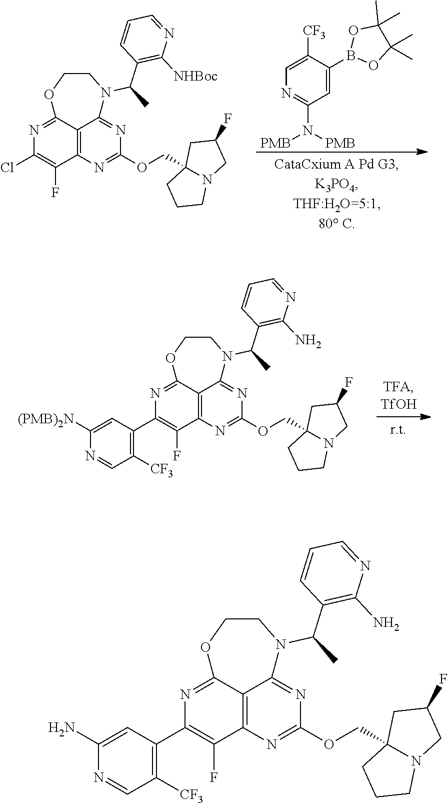

### Step 1: 4-bromo-N,N-bis(4-methoxybenzyl)pyridin-2-amine

To a solution of 4-bromopyridin-2-amine (4.0 g, 23.12 mmol) in N,N-dimethylformamide (80 mL) was added 4-methoxybenzyl chloride (11.29 mL, 83.23 mmol) and potassium tert-butoxide (2.6 g, 23.12 mmol) at 0° C., the mixture was stirred at 0° C. for 2.5 hours. After completion, the reaction was diluted with saturated ammonium chloride solution, extracted with ethyl acetate and washed with brine. The organic layer was dried over anhydrous sodium sulfate and concentrated under vacuum. The residue was purified by flash chromatography on silica gel eluting with petroleum ether/ethyl acetate (5:1) to afford 4-bromo-N,N-bis(4-methoxybenzyl)pyridin-2-amine (3.6 g, 8.01 mmol, 34.7% yield) as a white solid. LC-MS: (ESI, m/z): 413.0&415.0 [M+H]⁺.

### Step 2: 4-bromo-5-iodo-N,N-bis(4-methoxybenzyl)pyridin-2-amine

To solution of 4-bromo-N,N-bis(4-methoxybenzyl)pyridin-2-amine (1.0 g, 2.42 mmol) in acetic acid (10 mL) was added N-iodosuccinimide (0.5 g, 2.42 mmol), the mixture was stirred at 25° C. for 3 hours. After completion, the reaction mixture was diluted with ethyl acetate and adjusted to pH>7 with saturated sodium bicarbonate solution and extracted with ethyl acetate. The organic layers were dried over anhydrous sodium sulfate and concentrated under vacuum. The residue was purified by flash chromatography on silica gel eluting with petroleum ether/ethyl acetate (10:1) to afford 4-bromo-5-iodo-N,N-bis(4-methoxybenzyl)pyridin-2-amine (850.0 mg, 1.21 mmol, 50.2% yield) as a light yellow oil. LC-MS: (ESI, m/z): 539.0&541.0 [M+H]⁺.

### Step 3: 4-bromo-N,N-bis(4-methoxybenzyl)-5-(trifluoromethyl)pyridin-2-amine

Under nitrogen, a solution of 4-bromo-5-iodo-N,N-bis(4-methoxybenzyl)pyridin-2-amine (830.0 mg, 1.54 mmol), copper(I) iodide (2.93 g, 15.39 mmol) and methyl 2,2-difluoro-2-(fluorosulfonyl)acetate (2.95 g, 15.39 mmol) in N,N-dimethylformamide (8 mL) was stirred for 3.5 hours at 90° C. After completion, the mixture was cooled to ambient temperature. The resulting solution was extracted with ethyl acetate. The organic layers were washed with brine, dried over anhydrous sodium sulfate and concentrated under vacuum. The residue was purified by flash chromatography on silica gel eluting with petroleum ether/ethyl acetate (16:1) to afford 4-bromo-N,N-bis(4-methoxybenzyl)-5-(trifluoromethyl)pyridin-2-amine (299.0 mg, 0.57 mmol, 37.5% yield). LC-MS: (ESI, m/z): 481.1&483.1 [M+H]⁺.

### Step 4: N,N-bis(4-methoxybenzyl)-4-(4,4,5,5-tetramethyl-1,3,2-dioxaborolan-2-yl)-5-(trifluoromethyl)pyridin-2-amine

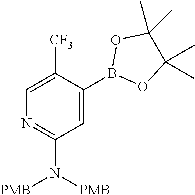

Under nitrogen, a solution of 4-bromo-N,N-bis(4-methoxybenzyl)-5-(trifluoromethyl)pyridin-2-amine (259.0 mg, 0.54 mmol), bis(pinacolato)diboron (819.9 mg, 3.23 mmol), [1,1′-bis(diphenylphosphino)ferrocene]dichloropalladium(II) (40.0 mg, 0.05 mmol) and potassium acetate (0.07 mL, 1.08 mmol) in 1,4-dioxane (1 mL) was stirred at 80° C. for 3.5 hours. After completion, the solids were filtered out. After filtration, the filtrate was concentrated under reduced pressure. The resulting residue was purified by reverse phase chromatography (acetonitrile/0.1% NH₄HCO₃ in water) to afford N,N-bis(4-methoxybenzyl)-4-(4,4,5,5-tetramethyl-1,3,2-dioxaborolan-2-yl)-5-(trifluoromethyl)pyridin-2-amine (280.0 mg, 0.42 mmol, 78.8% yield) as a yellow oil. LC-MS: (ESI, m/z): 529.2[M+H]⁺.

### Step 5: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-5-(trifluoromethyl)pyridin-2-amine

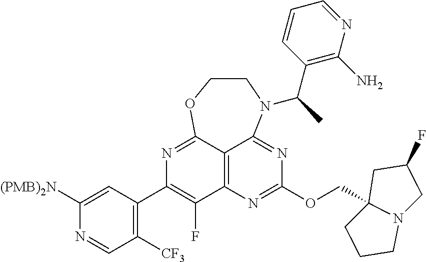

Under nitrogen, a solution of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (242.0 mg, 0.39 mmol), N,N-bis(4-methoxybenzyl)-4-(4,4,5,5-tetramethyl-1,3,2-dioxaborolan-2-yl)-5-(trifluoromethyl)pyridin-2-amine (248.3 mg, 0.47 mmol), methanesulfonato(diadamantyl-n-butylphosphino)-2′-amino-1,1′-biphenyl-2-yl)palladium(II) (28.5 mg, 0.04 mmol) and potassium phosphate (249.3 mg, 1.17 mmol) in tetrahydrofuran (3.5 mL) and water (0.7 mL) was stirred at 80° C. for 1 hour. After completion, the solution was diluted with water and extracted with ethyl acetate. The organic layers were washed with brine, dried over anhydrous sodium sulfate and concentrated. The residue was purified by flash chromatography on silica gel eluting with dichloromethane/methanol (30:1) to afford 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-5-(trifluoromethyl)pyridin-2-amine (270.0 mg, 0.26 mmol, 67.1% yield) as a brown solid. LC-MS: (ESI, m/z): 884.3 [M+H]⁺.

### Step 6: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-(trifluoromethyl)pyridin-2-amine

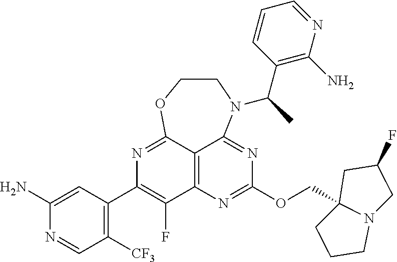 [SMILES: C[C@H](c1cccnc1N)N1CCOc2nc(-c3cc(N)ncc3C(F)(F)F)c(F)c3nc(OC[C@@]45CCCN4C[C@H](F)C5)nc1c23]

A solution of 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-5-(trifluoromethyl)pyridin-2-amine (260.0 mg, 0.29 mmol) and trifluoromethanesulfonic acid (0.3 mL) in 2,2,2-trifluoroacetic acid (3 mL) was stirred at 25° C. for 0.5 hours. After completion, the solvent was removed under vacuum. The resulting solution was diluted with dichloromethane and adjust PH to 7 with saturated sodium bicarbonate solution.

The resulting solution was extracted with dichloromethane. The organic layers were washed with brine, dried over anhydrous sodium sulfate and concentrated. The residue was purified by flash chromatography on silica gel eluting with dichloromethane/methanol (11:1) to afford crude product. The crude product was purified by Prep-HPLC with the following conditions: Column: YMC Triart C18 ExRs Sum, 30*150 mm; Mobile Phase A: Water(10 mmol/L NH₄HCO₃), Mobile Phase B: ACN; Flow rate: 60 mL/min mL/min; Gradient: 30% B to 42% B in 10 min; Wave Length: 254 nm/220 nm nm to afford 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-5-(trifluoromethyl)pyridin-2-amine (54.9 mg, 0.08 mmol, 28.7% yield) as a white solid. LC-MS: (ESI, m/z): 644.3 [M+H]⁺.

Example 608: ¹H NMR (300 MHz, DMSO-d₆, ppm) δ 8.34 (s, 1H), 7.97 (dd, J=4.9, 1.7 Hz, 1H), 7.62 (dd, J=7.6, 1.7 Hz, 1H), 6.96 (s, 2H), 6.67 (dd, J=7.5, 4.9 Hz, 1H), 6.44 (s, 1H), 6.34 (q, J=6.6 Hz, 1H), 5.68 (s, 2H), 5.29 (d, J=54.4 Hz, 1H), 4.43 (dd, J=12.7, 6.5 Hz, 1H), 4.29 (dd, J=12.7, 6.3 Hz, 1H), 4.13 (s, 2H), 3.72 (dd, J=16.0, 6.4 Hz, 1H), 3.43-3.34 (m, 1H), 3.15-2.97 (m, 3H), 2.91-2.72 (m, 1H), 2.21-2.13 (m, 1H), 2.13-1.97 (m, 2H), 1.92-1.68 (m, 3H), 1.56 (d, J=6.8 Hz, 3H). LC-MS: (ESI, m/z): 644.3 [M+H]⁺.

### Example 609: 3-((1R)-1-(5-(3-amino-2-fluoro-5-methyl-6-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyrazin-2-amine

### Synthetic Route

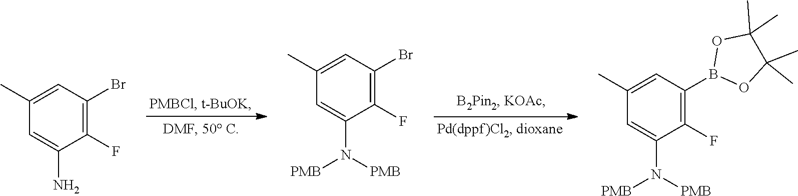

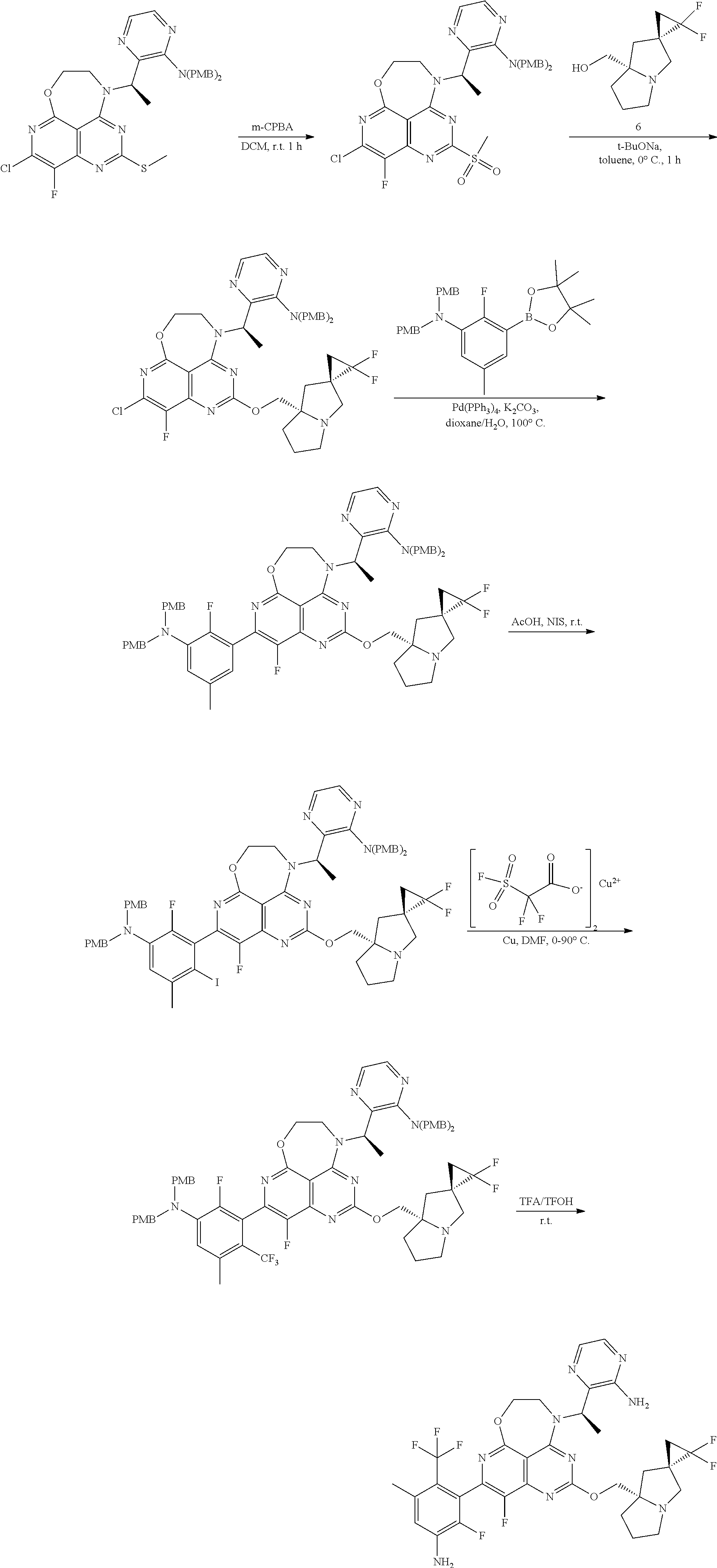

### Step 1: 3-bromo-2-fluoro-N,N-bis(4-methoxybenzyl)-5-methylaniline

To a solution of 3-bromo-2-fluoro-5-methyl-aniline (2.80 g, 13.72 mmol) and 4-methoxybenzylchloride (7.71 g, 49.40 mmol) in N,N-dimethylformamide (28 mL) was added potassium tert-butoxide (7.68 g, 68.61 mmol), the mixture was stirred at 50° C. for 1 hour. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (11) to afford 3-bromo-2-fluoro-N,N-bis(4-methoxybenzyl)-5-methylaniline (5.89 g, 10.84 mmol, 79% yield) as a yellow oil. LC-MS: (ESI, m/z): 444.1 [M+H]⁺.

### Step 2: 2-fluoro-N,N-bis(4-methoxybenzyl)-5-methyl-3-(4,4,5,5-tetramethyl-1,3,2-dioxaborolan-2-yl)aniline

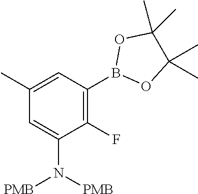

Under nitrogen, a solution of 3-bromo-2-fluoro-N,N-bis(4-methoxybenzyl)-5-methylaniline (4.0 g, 9.00 mmol), bis(pinacolato)diboron (6.86 g, 27.01 mmol), potassium acetate (1.69 mL, 27.01 mmol) and 1,1′-bis(diphenylphosphino)ferrocene]dichloropalladium(II) (653.25 mg, 0.90 mmol) in 1,4-dioxane (40 mL) was stirred at 110° C. for 2 hours. After completion, the reaction was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1:1) to afford 2-fluoro-N,N-bis(4-methoxybenzyl)-5-methyl-3-(4,4,5,5-tetramethyl-1,3,2-dioxaborolan-2-yl)aniline (5.0 g, 7.63 mmol, 84.8% yield) as a yellow solid. LC-MS: (ESI, m/z): 492.3 [M+H]⁺.

### Step 3: (R)-3-(1-(5-chloro-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyrazin-2-amine

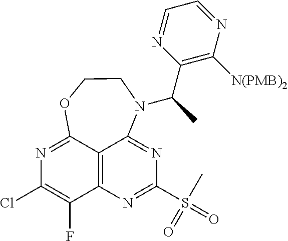

To a solution of (R)-3-(1-(5-chloro-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyrazin-2-amine (1.5 g, 2.31 mmol) in dichloromethane (15 mL) was added 3-chloroperoxybenzoic acid (998.4 mg, 5.79 mmol), the mixture was stirred at 25° C. for 0.5 hours. After completion, the reaction was quenched with saturated sodium thiosulfate solution. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1:1) to afford (R)-3-(1-(5-chloro-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyrazin-2-amine (1.15 g, 1.38 mmol, 59.9% yield) as a yellow solid. LC-MS: (ESI, m/z): 680.2 [M+H]⁺.

### Step 4: 3-((R)-1-(5-chloro-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine

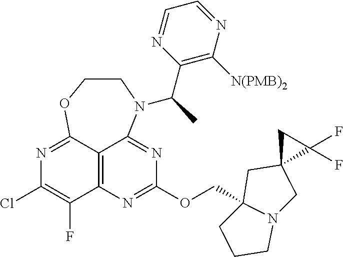

To a solution of ((1R,7a'S)-2,2-difluorodihydro-1′H,3′H-spiro[cyclopropane-1,2′-pyrrolizin]-7a′(5′H)-yl)methanol (747.0 mg, 3.68 mmol) in toluene (5 mL) was added sodium tert-butoxide (423.8 mg, 4.41 mmol), the mixture was stirred at 0° C. for 10 minutes. Then (R)-3-(1-(5-chloro-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyrazin-2-amine (1.0 g, 1.47 mmol) in toluene (5 mL) was added and stirred at 0° C. for 1 hour. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1:1) to afford 3-((R)-1-(5-chloro-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine (900.0 mg, 0.84 mmol, 57.2% yield) as a yellow solid. LC-MS: (ESI, m/z): 803.3 [M+H]⁺.

### Step 5: 3-((R)-1-(5-(3-(bis(4-methoxybenzyl)amino)-2-fluoro-5-methylphenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine

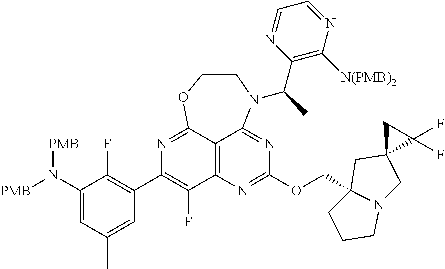

Under nitrogen, a solution of 3-((R)-1-(5-chloro-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine (850.0 mg, 1.06 mmol), 2-fluoro-N,N-bis(4-methoxybenzyl)-5-methyl-3-(4,4,5,5-tetramethyl-1,3,2-dioxaborolan-2-yl)aniline (1.56 g, 3.17 mmol), potassium carbonate (438.75 mg, 3.17 mmol) and tetrakis(triphenylphosphine)palladium (122.3 mg, 0.11 mmol) in 1,4-dioxane (10 mL) and water (2 mL) was stirred at 100° C. for 2 hours. After completion, the reaction was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1:1) to afford 3-((R)-1-(5-(3-(bis(4-methoxybenzyl)amino)-2-fluoro-5-methylphenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine (1.1 g, 0.58 mmol, 55.1% yield) as a yellow solid.LC-MS: (ESI, m/z): 1132.5 [M+H]⁺.

### Step 6: 3-((1R)-1-(5-(3-(bis(4-methoxybenzyl)amino)-2-fluoro-6-iodo-5-methylphenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine

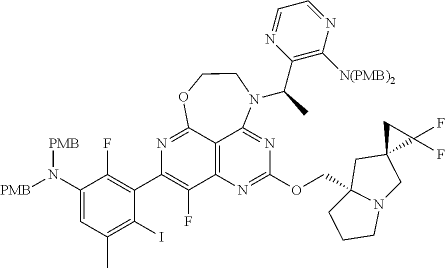

A mixture of 3-((R)-1-(5-(3-(bis(4-methoxybenzyl)amino)-2-fluoro-5-methylphenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine (750.0 mg, 0.7 mmol) and N-iodosuccinimide (163.9 mg, 0.7 mmol) in acetic acid (7 mL) was stirred at 25° C. for 30 minutes. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/ethyl acetate (7:3) to afford 3-((1R)-1-(5-(3-(bis(4-methoxybenzyl)amino)-2-fluoro-6-iodo-5-methylphenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine (480.0 mg, 0.4 mmol, 57.6% yield) as a yellow solid. LC-MS: (ESI, m/z): 1258.4[M+H]⁺.

### Step 7: 3-((1R)-1-(5-(3-(bis(4-methoxybenzyl)amino)-2-fluoro-5-methyl-6-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine

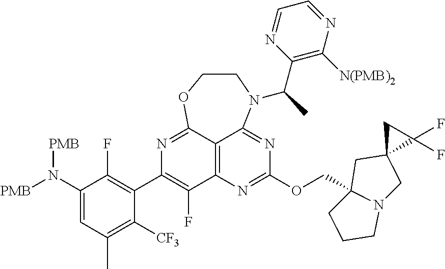

Under nitrogen, a solution of 3-((1R)-1-(5-(3-(bis(4-methoxybenzyl)amino)-2-fluoro-6-iodo-5-methylphenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine (372.0 mg, 0.3 mmol) and copper (189.2 mg, 2.9 mmol) in N,N-dimethylformamide (4 mL) was added bis[(2,2-difluoro-2-fluorosulfonyl-acetyl)oxy]copper (711.4 mg, 2.9 mmol) at −60° C. for 10 minutes, then the reaction mixture was stirred at 80° C. for 30 minutes. After completion, the solids were filtered out. After filtration, the filtrate was concentrated under reduced pressure. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (95:5) to afford 3-((1R)-1-(5-(3-(bis(4-methoxybenzyl)amino)-2-fluoro-5-methyl-6-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine (277.0 mg, 0.2 mmol, 78.1% yield) as a yellow solid. LC-MS: (ESI, m/z): 1200.5[M+H]⁺.

### Step 8: 3-((1R)-1-(5-(3-amino-2-fluoro-5-methyl-6-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyrazin-2-amine

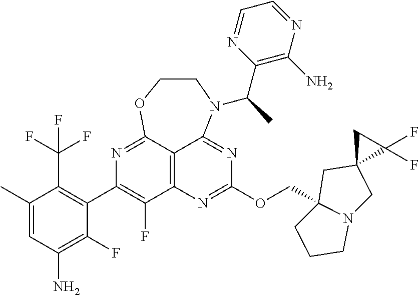 [SMILES: Cc1cc(N)c(F)c(-c2nc3c4c(nc(OC[C@@]56CCCN5C[C@]5(CC5(F)F)C6)nc4c2F)N([C@H](C)c2nccnc2N)CCO3)c1C(F)(F)F]

A mixture of 3-((1R)-1-(5-(3-(bis(4-methoxybenzyl)amino)-2-fluoro-5-methyl-6-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine (240.0 mg, 0.3 mmol) in trifluoromethanesulfonic acid (0.3 mL) and 2,2,2-trifluoroacetic acid (3 mL) was stirred at 25° C. for 30 minutes. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (95:5) to afford crude product. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 42% B to 72% B in 8 min; Wave Length: 254 nm/220 nm to afford 3-((1R)-1-(5-(3-amino-2-fluoro-5-methyl-6-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyrazin-2-amine (46.5 mg, 0.1 mmol, 22.5% yield) as a white solid. LC-MS: (ESI, m/z): 720.4 [M+H]⁺

Example 609: ¹H NMR (300 MHz, DMSO-d₆, ppm) δ 7.95 (t, J=2.3 Hz, 1H), 7.79 (t, J=2.5 Hz, 1H), 6.81 (d, J=8.8 Hz, 1H), 6.42-6.37 (m, 3H), 5.99 (s, 2H), 4.57-4.48 (m, 1H), 4.44-4.36 (m, 1H), 4.27 (dd, J=10.5, 3.6 Hz, 1H), 4.15 (dd, J=10.5, 7.5 Hz, 1H), 3.93-3.85 (m, 1H), 3.67 (td, J=15.9, 6.1Hz, 1H), 3.12-2.97 (m, 2H), 2.70 (d, J=12.0 Hz, 1H), 2.59-2.52 (m, 1H), 2.34 (d, J=2.7 Hz, 3H), 2.09-1.86 (m, 3H), 1.83-1.71 (m, 2H), 1.65-1.42 (m, 6H).

### Example 612: 3-((R)-1-(5-(5-amino-2-(2,2-difluoroethyl)-4-fluoro-3-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Synthetic Route

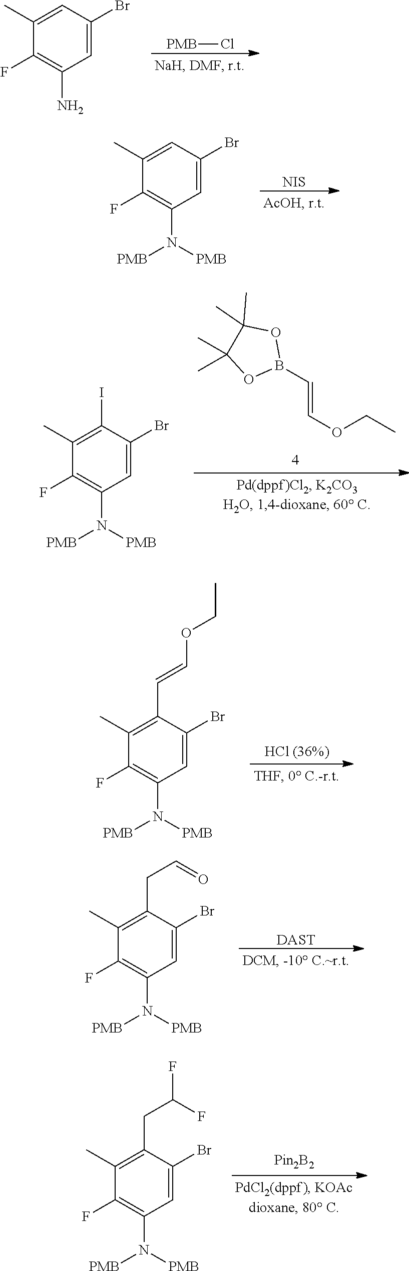

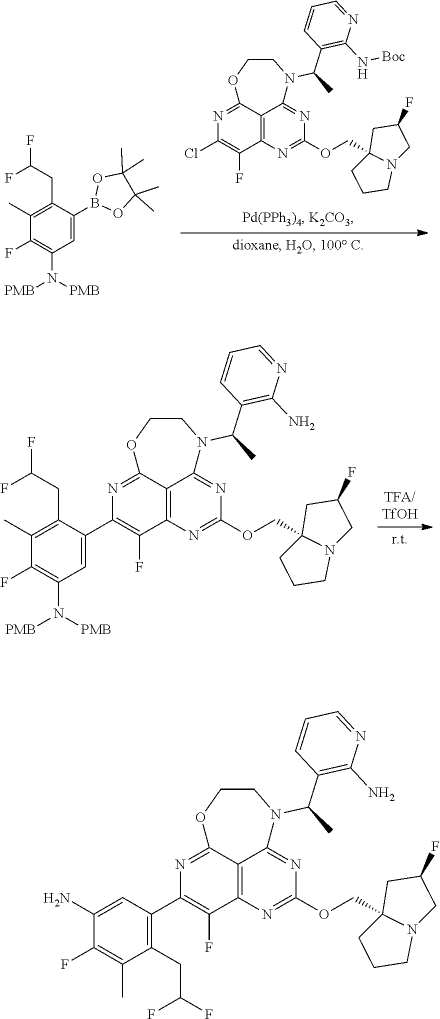

### Step 1: 5-bromo-2-fluoro-N,N-bis(4-methoxybenzyl)-3-methylaniline

To a solution of 5-bromo-2-fluoro-3-methylaniline (4.70 g, 23.03 mmol) and 4-methoxybenzyl chloride (9.02 g, 57.59 mmol) in N,N-dimethylformamide (37 mL) added sodium hydride (3.84 mL, 115.17 mmol, 60% dispersion in mineral oil) at 0° C., the mixture was stirred at 25° C. for 2 hours. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (96:4) to afford 5-bromo-2-fluoro-N,N-bis(4-methoxybenzyl)-3-methylaniline (9.50 g, 20.95 mmol, 91% yield) as a yellow solid. LC-MS: (ESI, m/z): 444.1 [M+H]⁺.

### Step 2: 5-bromo-2-fluoro-4-iodo-N,N-bis(4-methoxybenzyl)-3-methylaniline

A solution of 5-bromo-2-fluoro-N,N-bis(4-methoxybenzyl)-3-methylaniline (8.60 g, 19.35 mmol) and N-iodosuccinimide (5.23 g, 23.23 mmol) in acetic acid (80 mL) was stirred at 25° C. for 3 hours. After completion, the reaction was quenched with saturated sodium thiosulfate solution, extracted with dichloromethane and the organic layers were combined. The organic layer was dried over anhydrous sodium sulfate and concentrated under vacuum. The residue was purified by flash chromatography on reverse-phase column eluting with water/acetonitrile (7:3) to afford 5-bromo-2-fluoro-4-iodo-N,N-bis(4-methoxybenzyl)-3-methylaniline (6.50 g, 11.29 mmol, 58.3% yield) as a yellow solid. LC-MS: (ESI, m/z): 570.0 [M+H]⁺.

### Step 3: (E)-5-bromo-4-(2-ethoxyvinyl)-2-fluoro-N,N-bis(4-methoxybenzyl)-3-methylaniline

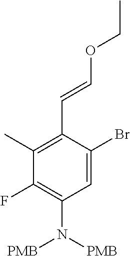

Under nitrogen, a solution of 5-bromo-2-fluoro-4-iodo-N,N-bis(4-methoxybenzyl)-3-methylaniline (1.50 g, 2.63 mmol), potassium carbonate (1.10 g, 7.89 mmol), 1,1′-bis(diphenylphosphino)ferrocene-palladium(II)dichloride dichloromethane complex (195.1 mg, 0.26 mmol) and (E)-2-(2-ethoxyvinyl)-4,4,5,5-tetramethyl-1,3,2-dioxaborolane (1.04 g, 5.26 mmol) in 1,4-dioxane (10 ml) and water (1 ml) was stirred at 60° C. for 16 hours. After completion, the resulting solution was diluted with ethyl acetate and washed with water. The organic layers were combined, dried over anhydrous sodium sulfate and concentrated under vacuum. The residue was purified by flash chromatography on silica gel eluting with petroleum ether/ethyl acetate (95:5) to afford (E)-5-bromo-4-(2-ethoxyvinyl)-2-fluoro-N,N-bis(4-methoxybenzyl)-3-methylaniline (550.0 mg, 0.71 mmol, 26.8% yield) as a brown oil. LC-MS: (ESI, m/z): 514.1 [M+H]⁺.

### Step 4: 2-(4-(bis(4-methoxybenzyl)amino)-6-bromo-3-fluoro-2-methylphenyl)acetaldehyde

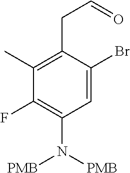

To a solution of (E)-5-bromo-4-(2-ethoxyvinyl)-2-fluoro-N,N-bis(4-methoxybenzyl)-3-methylaniline (500.0 mg, 0.97 mmol) in tetrahydrofuran (5 mL) was added hydrochloric acid (1 mL, 1 mol/L), the mixture was stirred at 25° C. for 2 hours. After completion, the reaction mixture was concentrated under vacuum and diluted with dichloromethane, adjusted to pH>7 with sodium bicarbonate saturated solution, extracted with dichloromethane and the organic layers were combined. The organic layer was dried over anhydrous sodium sulfate and concentrated under vacuum to afford 2-(4-(bis(4-methoxybenzyl)amino)-6-bromo-3-fluoro-2-methylphenyl)acetaldehyde (270.0 mg, 0.39 mmol, 40% yield) as a brown oil. LC-MS: (ESI, m/z): 486.1 [M+H]⁺.

### Step 5: 5-bromo-4-(2,2-difluoroethyl)-2-fluoro-N,N-bis(4-methoxybenzyl)-3-methylaniline

To a solution of 2-(4-(bis(4-methoxybenzyl)amino)-6-bromo-3-fluoro-2-methylphenyl)acetaldehyde (260.0 mg, 0.53 mmol) in dichloromethane (5 mL) was added diethylaminosulfur trifluoride (689.1 mg, 4.28 mmol) at −10° C., the mixture was stirred at −10° C. for 10 minutes. Then the mixture was stirred for 1 hour at 25° C. After completion, the reaction was quenched with methanol, diluted with dichloromethane, washed with water and the organic layer was combined. The organic layer was dried over anhydrous sodium sulfate and concentrated under vacuum. The residue was purified by flash chromatography on silica gel eluting with petroleum ether/ethyl acetate (93:7) to afford 5-bromo-4-(2,2-difluoroethyl)-2-fluoro-N,N-bis(4-methoxybenzyl)-3-methylaniline (190 mg, 0.36 mmol, 66.4% yield) as a white solid. LC-MS: (ESI, m/z): 508.1 [M+H]⁺.

### Step 6: 4-(2,2-difluoroethyl)-2-fluoro-N,N-bis(4-methoxybenzyl)-3-methyl-5-(4,4,5,5-tetramethyl-1,3,2-dioxaborolan-2-yl)aniline

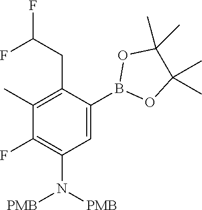

Under nitrogen, a solution of 5-bromo-4-(2,2-difluoroethyl)-2-fluoro-N,N-bis(4-methoxybenzyl)-3-methylaniline (1.09 g, 2.14 mmol), potassium acetate (630.1 mg, 6.43 mmol), bis(pinacolato)diboron (1.63 g, 6.43 mmol) and 1,1′-bis(diphenylphosphino)ferrocene-Palladium(II)dichloride dichloromethane complex (159.1 mg, 0.21 mmol) in 1,4-dioxane (15 mL) was stirred at 80° C. for 16 hours. After completion, the solvent was concentrated under vacuum and diluted with dichloromethane and filtered. The filtrate was combined and concentrated under vacuum. The residue was purified by flash chromatography on silica gel eluting with petroleum ether/ethyl acetate (90:10) to afford 4-(2,2-difluoroethyl)-2-fluoro-N,N-bis(4-methoxybenzyl)-3-methyl-5-(4,4,5,5-tetramethyl-1,3,2-dioxaborolan-2-yl)aniline (850.0 mg, 1.38 mmol, 64.2% yield) as a black oil. LC-MS: (ESI, m/z): 556.3 [M+H]⁺.

### Step 7: 3-((R)-1-(5-(5-(bis(4-methoxybenzyl)amino)-2-(2,2-difluoroethyl)-4-fluoro-3-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

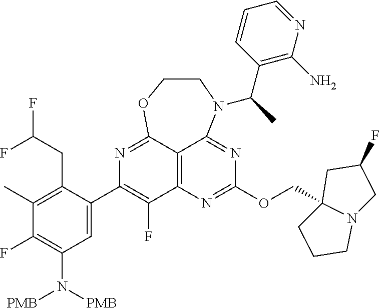

Under nitrogen, a solution of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (350.0 mg, 0.57 mmol), 4-(2,2-difluoroethyl)-2-fluoro-N,N-bis(4-methoxybenzyl)-3-methyl-5-(4,4,5,5-tetramethyl-1,3,2-dioxaborolan-2-yl)aniline (629.07 mg, 1.13 mmol), tetrakis(triphenylphosphine)palladium (65.4 mg, 0.06 mmol) and patassium carbonate (234.8 mg, 1.70 mmol) in 1,4-dioxane (3 mL) and water (0.6 mL) was stirred for 3 hours at 100° C. After completion, the reaction was diluted with dichloromethane, washed with brine and the organic layers were combined. The organic layer was dried over anhydrous sodium sulfate and concentrated under vacuum. The residue was purified by flash chromatography on reverse-phase column eluting with water/acetonitrile (6:4) to afford 3-((R)-1-(5-(5-(bis(4-methoxybenzyl)amino)-2-(2,2-difluoroethyl)-4-fluoro-3-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (120.0 mg, 0.09 mmol, 16.3% yield) as a solid. LC-MS: (ESI, m/z): 911.4 [M+H]⁺.

### Step 8: 3-((R)-1-(5-(5-amino-2-(2,2-difluoroethyl)-4-fluoro-3-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

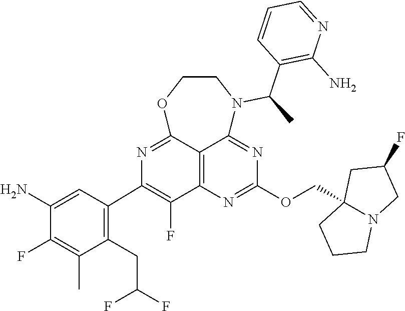 [SMILES: Cc1c(F)c(N)cc(-c2nc3c4c(nc(OC[C@@]56CCCN5C[C@H](F)C6)nc4c2F)N([C@H](C)c2cccnc2N)CCO3)c1CC(F)F]

A solution of 3-((R)-1-(5-(5-(bis(4-methoxybenzyl)amino)-2-(2,2-difluoroethyl)-4-fluoro-3-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (120.0 mg, 0.13 mmol) in trifluoromethanesulfonic acid (0.2 mL) and trifluoroacetic acid (2 mL) was stirred for 16 hours at 25° C. After completion, the reaction mixture was concentrated under vacuum and diluted with dichloromethane, adjusted to pH>7 with sodium bicarbonate saturated solution, extracted with dichloromethane and the organic layers were combined. The organic layer was dried over anhydrous sodium sulfate and concentrated under vacuum. The residue was purified by flash chromatography on silica gel eluting with dichloromethane/methanol (91:9) to afford product. The product was further purified by Prep-HPLC with the following conditions: Column: XSelect CSH Fluoro Phenyl 30*150 mm, 5 μm; Mobile Phase A: Water(10 mmol/L NH₄HCO₃), Mobile Phase B: ACN; Flow rate: 60 mL/min; Gradient: 17% B to 42% B in 9 min; Wave Length: 254 nm/220 nm; RT1(min): 8.5 to afford 3-((R)-1-(5-(5-amino-2-(2,2-difluoroethyl)-4-fluoro-3-methylphenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (20.6 mg, 0.03 mmol, 23.2% yield) as a white solid. LC-MS: (ESI, m/z): 671.3 [M+H]⁺.

Example 612: ¹H NMR (300 MHz, Methanol-d₄) δ 7.98 (dd, J=5.1, 1.7 Hz, 1H), 7.77 (d, J=8.0 Hz, 1H), 6.80 (dd, J=7.5, 5.1Hz, 1H), 6.73 (d, J=8.9 Hz, 1H), 6.62 (dd, J=6.9 Hz, 1H), 6.12-5.62 (m, 1H), 5.33 (d, J=53.8 Hz, 1H), 4.47 (dd, J=12.8, 6.3 Hz, 1H), 4.42-4.18 (m, 3H), 3.77 (dd, J=15.9, 6.4 Hz, 1H), 3.55 (dd, J=15.9, 6.4 Hz, 1H), 3.29-3.14 (m, 2H), 3.14-2.94 (m, 4H), 2.47-2.27 (m, 4H), 2.27-2.11 (m, 2H), 2.10-1.78 (m, 3H), 1.67 (d, J=6.9 Hz, 3H). LC-MS: (ESI, m/z): 671.3 [M+H]⁺.

### Example 613: 5-((R)-1-(5-(6-amino-5-fluoro-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrimidin-4-amine

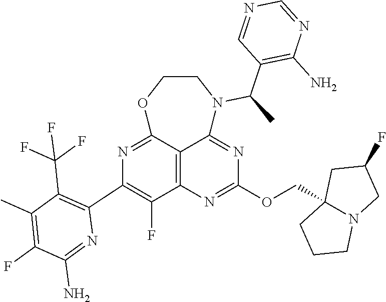 [SMILES: Cc1c(F)c(N)nc(-c2nc3c4c(nc(OC[C@@]56CCCN5C[C@H](F)C6)nc4c2F)N([C@H](C)c2cncnc2N)CCO3)c1C(F)(F)F]

### Synthetic Route

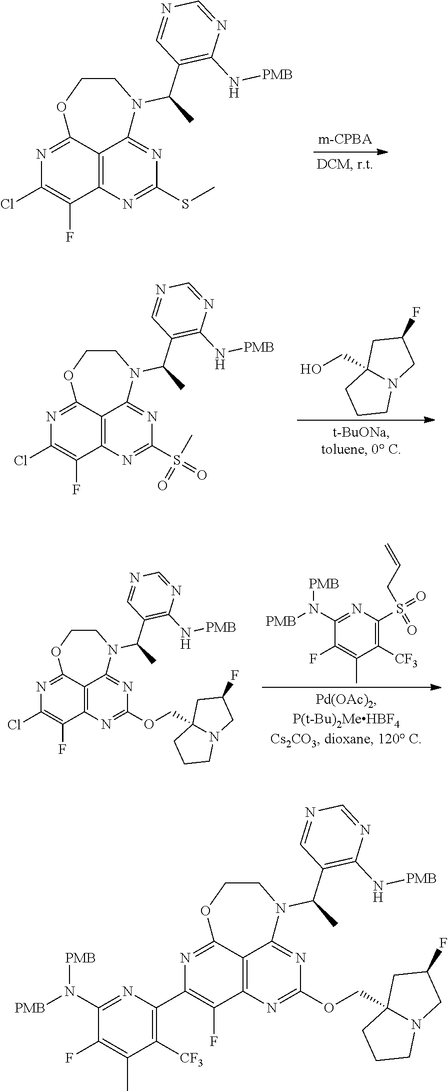

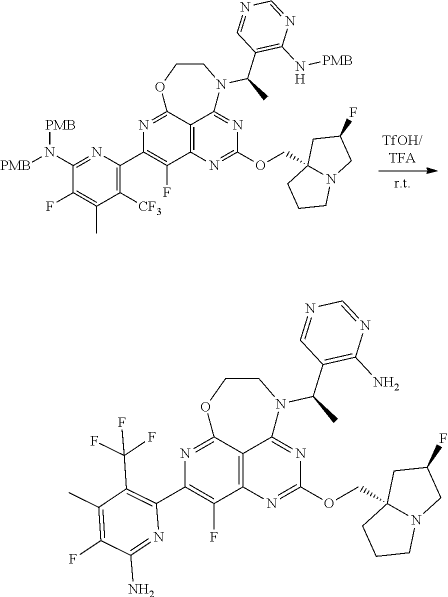

### Step 1: (R)-5-(1-(5-chloro-4-fluoro-2-(methylsulfonyl)-8,9-dihydro-10H-7-oxa-1,3,6,10-tetraazacyclohepta[de]naphthalen-10-yl)ethyl)-N-(4-methoxybenzyl)pyrimidin-4-amine

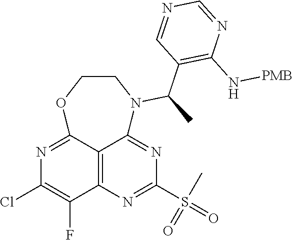

To a solution of (R)-5-(1-(5-chloro-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrimidin-4-amine (300.0 mg, 0.57 mmol) in dichloromethane (3 mL) was added 3-chloroperoxybenzoicacid (156.9 mg, 0.91 mmol) at 0° C. and stirred at 25° C. for 0.5 hours. After completion, the resulting solution was quenched with saturated sodium bisulfite solution and extracted with dichloromethane. Then the organic layers were combined, washed with brine, dried over anhydrous sodium sulfate and concentrated under vacuum. The residue was purified by flash chromatography on silica gel eluting with dichloromethane/methanol (95:5) to afford (R)-5-(1-(5-chloro-4-fluoro-2-(methylsulfonyl)-8,9-dihydro-10H-7-oxa-1,3,6,10-tetraazacyclohepta[de]naphtha len-10-yl)ethyl)-N-(4-methoxybenzyl)pyrimidin-4-amine (207.0 mg, 0.33 mmol, 57.3% yield) as a yellow solid. LCMS (ESI, m/z): 560.1 [M+H]⁺.

### Step 2: 5-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrimidin-4-amine

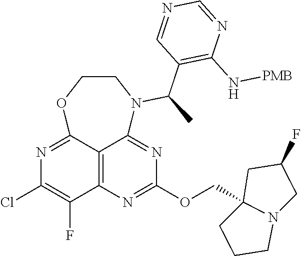

Sodium tert-butoxide (212.0 mg, 2.21 mmol) was added to a solution of ((2R,7aS)-2-fluorotetrahydro-1H-pyrrolizin-7a(5H)-yl)methanol (234.1 mg, 1.47 mmol) in toluene (4 mL) at 0° C. and was stirred for 10 minutes at 0° C. Then (R)-5-(1-(5-chloro-4-fluoro-2-(methylsulfonyl)-8,9-dihydro-10H-7-oxa-1,3,6,10-tetraazacyclohepta[de]naphthalen-10-yl)ethyl)-N-(4-methoxybenzyl)pyrimidin-4-amine (400.0 mg, 0.74 mmol) was added and stirred at 0° C. for 10 minutes. After completion, the resulting solution was diluted with ethyl acetate and washed with water. The organic layer was dried over anhydrous sodium sulfate and concentrated under vacuum. The residue was purified by flash chromatography on silica gel eluting with dichloromethane/methanol (93:7) to afford 5-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrimidin-4-amine (290.0 mg, 0.36 mmol, 50.7% yield) as a white solid. LCMS (ESI, m/z): 639.2 [M+H]⁺.

### Step 3: 5-((R)-1-(5-(6-(bis(4-methoxybenzyl)amino)-5-fluoro-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrimidin-4-amine

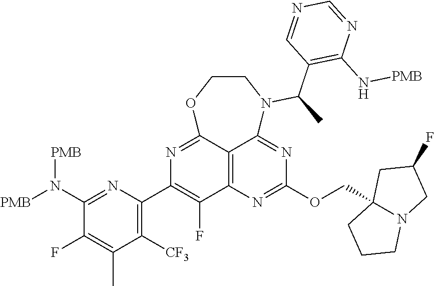

Under nitrogen, a solution of 5-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrimidin-4-amine (280.0 mg, 0.44 mmol), 6-allylsulfonyl-3-fluoro-N,N-bis[(4-methoxyphenyl)methyl]-4-methyl-5-(trifluoromethyl)pyridin-2-amine (353.9 mg, 0.66 mmol), cesium carbonate (287.3 mg, 0.88 mmol), palladium(II)acetate (9.9 mg, 0.04 mmol) and di-tert-butylmethylphosphonium tetrafluoroborate (21.7 mg, 0.09 mmol) in 1,4-dioxane (3 mL) was stirred at 120° C. for 3 hours. After completion, the resulting solution was diluted with ethyl acetate and washed with water. The organic layer was dried over anhydrous sodium sulfate and concentrated under vacuum. The residue was purified by flash chromatography on silica gel eluting with petroleum ether/ethyl acetate (1:1) to afford 5-((R)-1-(5-(6-(bis(4-methoxybenzyl)amino)-5-fluoro-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrimidin-4-amine (136.0 mg, 0.05 mmol, 12% yield) as a yellow solid. LCMS (ESI, m/z): 1037.4 [M+H]⁺.

### Step 4: 5-((R)-1-(5-(6-amino-5-fluoro-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrimidin-4-amine

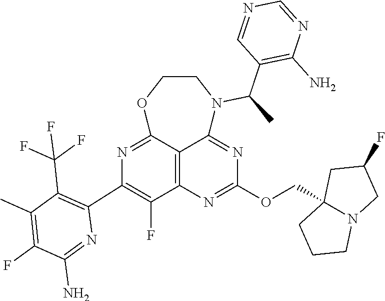 [SMILES: Cc1c(F)c(N)nc(-c2nc3c4c(nc(OC[C@@]56CCCN5C[C@H](F)C6)nc4c2F)N([C@H](C)c2cncnc2N)CCO3)c1C(F)(F)F]

A solution of 5-((R)-1-(5-(6-(bis(4-methoxybenzyl)amino)-5-fluoro-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrimidin-4-amine (130.0 mg, 0.05 mmol) in trifluoromethanesulfonic acid (0.1 mL) and 2,2,2-trifluoroacetic acid (1 mL) was stirred at 25° C. for 1 hour. After completion, the resulting solution was concentrated under vacuum, diluted with dichloromethane and adjusted pH to 7 with saturated sodium bicarbonate solution. The resulting solution was extracted with dichloromethane. The organic layers were dried over anhydrous sodium sulfate and concentrated to afford crude product. The crude product was purified by Prep-HPLC with the following conditions: Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 29% B to 51% B in 10 min; Wave Length: 254 nm/220 nm; RT1(min): 9 to afford 5-((R)-1-(5-(6-amino-5-fluoro-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrimidin-4-amine (11.2 mg, 0.02 mmol, 12.7% yield) as a white solid. LCMS: (ESI, m/z): 677.4 [M+H]⁺.

Example 613: ¹H NMR (400 MHz, DMSO-d₆, ppm) δ 8.38 (s, 1H), 8.29 (s, 1H), 7.15 (s, 2H), 6.75 (s, 2H), 6.24 (q, J=6.9 Hz, 1H), 5.29 (d, J=54.3 Hz, 1H), 4.56-4.41 (m, 1H), 4.40-4.27 (m, 1H), 4.10 (s, 2H), 3.87-3.72 (m, 1H), 3.58-3.41 (m, 1H), 3.18-2.93 (m, 3H), 2.89-2.75 (m, 1H), 2.41-2.24 (m, 3H), 2.22-1.93 (m, 3H), 1.93-1.70 (m, 3H), 1.59 (d, J=6.9 Hz, 3H).

### Example 614: 6-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-3-fluoro-4-methyl-5-(trifluoromethyl)pyridin-2-amine

### Synthetic Route

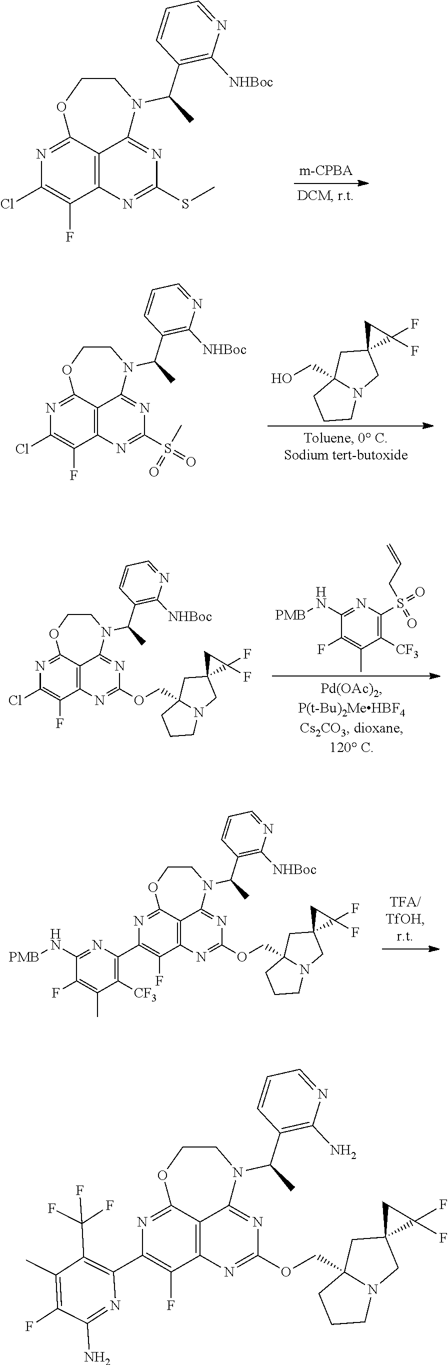

### Step 1: tert-butyl (R)-(3-(1-(5-chloro-4-fluoro-2-(methylsulfonyl)-8,9-dihydro-10H-7-oxa-1,3,6,10-tetraazacyclohepta[de]naphthalen-10-yl)ethyl)pyridin-2-yl)carbamate

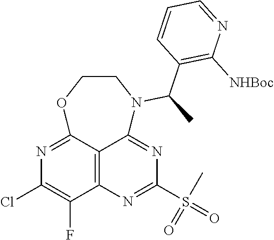 [SMILES: C[C@H](c1cccnc1NC(=O)OC(C)(C)C)N1CCOc2nc(Cl)c(F)c3nc(S(C)(=O)=O)nc1c23]

To a solution of tert-butyl (R)-(3-(1-(5-chloro-4-fluoro-2-(methylthio)-8,9-dihydro-10H-7-oxa-1,3,6,10-tetraazacyclohepta[de]naphthalen-10-yl)ethyl)pyridin-2-yl)carbamate (2.00 g, 3.29 mmol) in dichloromethane (20 mL) was added 3-chloroperoxybenzoic acid (1.42 g, 8.24 mmol) at 0° C. and stirred at 25° C. for 1 hour. After completion, the reaction was quenched with saturated sodium sulfite solution, diluted with dichloromethane and washed with water. The organic layer was dried over anhydrous sodium sulfate and concentrated under vacuum. The residue was purified by flash chromatography on silica gel eluting with petroleum ether/ethyl acetate (30:70) to afford tert-butyl (R)-(3-(1-(5-chloro-4-fluoro-2-(methylsulfonyl)-8,9-dihydro-10H-7-oxa-1,3,6,10-tetraazacyclohepta[de]naphthalen-10-yl)ethyl)pyridin-2-yl)carbamate (1.30 g, 2.03 mmol, 51.4% yield) as a yellow solid. LC-MS: (ESI, m/z): 539.1 [M+H]⁺.

### Step 2: tert-butyl (3-((R)-1-(5-chloro-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyridin-2-yl)carbamate

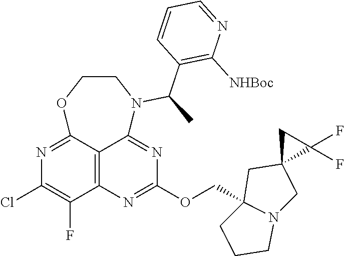 [SMILES: C[C@H](c1cccnc1NC(=O)OC(C)(C)C)N1CCOc2nc(Cl)c(F)c3nc(OC[C@@]45CCCN4C[C@]4(CC4(F)F)C5)nc1c23]

Under nitrogen, to a mixture of ((1R,7a'S)-2,2-difluorodihydro-1′H,3′H-spiro[cyclopropane-1,2′-pyrrolizin]-7a′(5′H)-yl)methanol (603.3 mg, 2.97 mmol) in toluene (8 mL) was added sodium tert-butoxide (427.5 mg, 4.45 mmol) and stirred for 10 minutes at 0° C. Then tert-butyl (R)-(3-(1-(5-chloro-4-fluoro-2-(methylsulfonyl)-8,9-dihydro-10H-7-oxa-1,3,6,10-tetraazacyclohepta[de]naphthalen-10-yl)ethyl)pyridin-2-yl)carbamate (800.0 mg, 1.48 mmol) was added and stirred for 15 minutes at 0° C. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (85:15) to afford tert-butyl (3-((R)-1-(5-chloro-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyridin-2-yl)carbamate (480.0 mg, 0.73 mmol, 48.8% yield) as a yellow solid. LC-MS: (ESI, m/z): 662.2 [M+H]⁺.

### Step 3: tert-butyl (3-((R)-1-(2-(((1R,7a'S)-2,2-difluorodihydro-1′H,3′H-spiro[cyclopropane-1,2′-pyrrolizin]-7a′(5′H)-yl)methoxy)-4-fluoro-5-(5-fluoro-6-((4-methoxybenzyl)amino)-4-methyl-3-(trifluoromethyl)pyridin-2-yl)-8,9-dihydro-10H-cyclohepta[de]naphthalen-10-yl)ethyl)pyridin-2-yl)carbamate

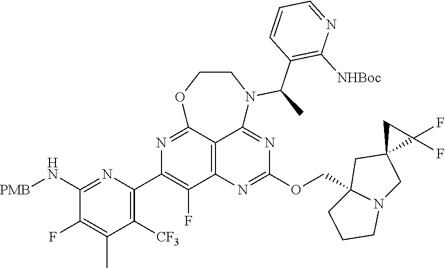

Under nitrogen, a solution of tert-butyl (3-((R)-1-(5-chloro-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyridin-2-yl)carbamate (540.0 mg, 0.82 mmol), tri-tert-butylphosphine tetrafluoroborate (40.5 mg, 0.16 mmol), 6-(allylsulfonyl)-3-fluoro-N-(4-methoxybenzyl)-4-methyl-5-(trifluoromethyl)pyridin-2-amine (511.9 mg, 1.22 mmol), palladium(II)acetate (18.5 mg, 0.08 mmol) and cesium carbonate (534.8 mg, 1.63 mmol) in 1,4-dioxane (6 mL) was stirred at 120° C. for 16 hours. After completion, the reaction mixture was concentrated under vacuum, diluted with dichloromethane, washed with water and the organic layer was combined. The organic layer was dried over anhydrous sodium sulfate and concentrated under vacuum. The residue was purified by flash chromatography on silica gel eluting with dichloromethane/methanol (95:5) to afford tert-butyl (3-((R)-1-(2-(((1R,7a'S)-2,2-difluorodihydro-1′H,3′H-spiro[cyclopropane-1,2′-pyrrolizin]-7a′(5′H)-yl)methoxy)-4-fluoro-5-(5-fluoro-6-((4-methoxybenzyl)amino)-4-methyl-3-(trifluoromethyl)pyridin-2-yl)-8,9-dihydro-10H-cyclohepta[de]naphthalen-10-yl)ethyl)pyridin-2-yl)carbamate (330.0 mg, 0.39 mmol, 48.2% yield) as a white solid. LC-MS: (ESI, m/z): 940.3 [M+H]⁺.

### Step 4: 6-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-3-fluoro-4-methyl-5-(trifluoromethyl)pyridin-2-amine

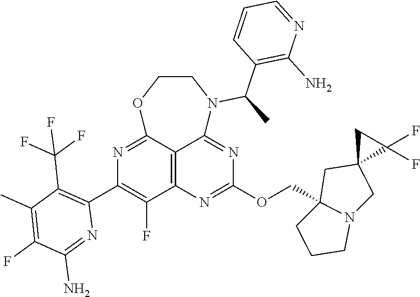 [SMILES: Cc1c(F)c(N)nc(-c2nc3c4c(nc(OC[C@@]56CCCN5C[C@]5(CC5(F)F)C6)nc4c2F)N([C@H](C)c2cccnc2N)CCO3)c1C(F)(F)F]

A solution of tert-butyl (3-((R)-1-(2-(((1R,7a'S)-2,2-difluorodihydro-1′H,3′H-spiro[cyclopropane-1,2′-pyrrolizin]-7a′(5′H)-yl)methoxy)-4-fluoro-5-(5-fluoro-6-((4-methoxybenzyl)amino)-4-methyl-3-(trifluoromethyl)pyridin-2-yl)-8,9-dihydro-10H-cyclohepta[de]naphthalen-10-yl)ethyl)pyridin-2-yl)carbamate (330.0 mg, 0.39 mmol) in trifluoromethanesulfonic acid (0.4 mL) and trifluoroacetic acid (4 mL) was stirred at 25° C. for 0.5 hours. After completion, the reaction mixture was concentrated under vacuum and diluted with dichloromethane, adjusted to pH>7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (90:10) to afford crude product. The product was further purified by Prep-HPLC with the following conditions: Column: XSelect CSH Fluoro Phenyl 30*150 mm, 5 μm; Mobile Phase A: Water(10 mmol/L NH₄HCO₃), Mobile Phase B: ACN; Flow rate: 60 mL/min; Gradient: 5% B to 20% B in 9 min; Wave Length: 254 nm/220 nm; RT1(min): 8.6 to afford 6-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-3-fluoro-4-methyl-5-(trifluoromethyl)pyridin-2-amine (28.7 mg, 0.04 mmol, 10.1% yield) as a white solid. LC-MS: (ESI, m/z): 720.2 [M+H]⁺.

Example 614: ¹H NMR (300 MHz, Methanol-d₄, ppm) δ 7.98 (dd, J=5.1, 1.6 Hz, 1H), 7.83-7.70 (m, 1H), 6.80 (dd, J=7.5, 5.1Hz, 1H), 6.63 (q, J=6.9 Hz, 1H), 4.56-4.29 (m, 4H), 3.88-3.46 (m, 2H), 3.28 (d, J=6.9 Hz, 1H), 3.23-3.10 (m, 1H), 2.86 (d, J=12.2 Hz, 1H), 2.79-2.62 (m, 1H), 2.52-2.36 (m, 3H), 2.30 (dd, J=13.5, 6.2 Hz, 1H), 2.18 (dd, J=11.2, 6.3 Hz, 1H), 2.04 (d, J=13.5 Hz, 1H), 2.00-1.88 (m, 2H), 1.87-1.73 (m, 1H), 1.68 (d, J=6.9 Hz, 3H), 1.56-1.35 (m, 2H). LC-MS: (ESI, m/z): 720.2 [M+H]⁺.

Example 615: 4-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-6-methyl-5-(trifluoromethyl)pyridin-2-amine

### Synthetic Route

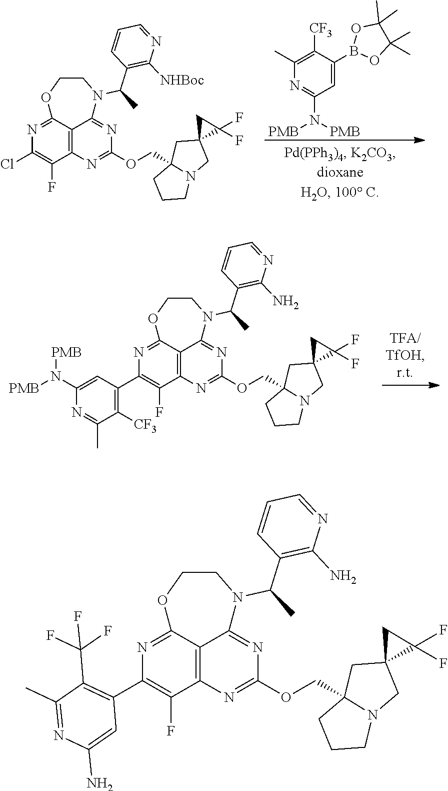

### Step 1: 4-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-N,N-bis(4-methoxybenzyl)-6-methyl-5-(trifluoromethyl)pyridin-2-amine

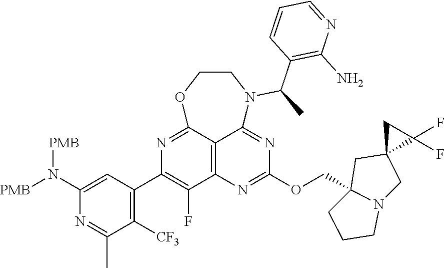

Under nitrogen, a solution of tert-butyl (3-((R)-1-(5-chloro-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyridin-2-yl)carbamate (200.0 mg, 0.30 mmol), potassium carbonate (125.3 mg, 0.91 mmol), N,N-bis(4-methoxybenzyl)-6-methyl-4-(4,4,5,5-tetramethyl-1,3,2-dioxaborolan-2-yl)-5-(trifluoromethyl)pyridin-2-amine (327.7 mg, 0.60 mmol) and tetrakis(triphenylphosphine)palladium (34.9 mg, 0.03 mmol) in 1,4-dioxane (2 mL) and water (0.4 mL) was stirred at 100° C. for 2 hours. After completion, the reaction mixture was concentrated under vacuum, diluted with dichloromethane, washed with water and the organic layer was combined. The organic layer was dried over anhydrous sodium sulfate and concentrated under vacuum. The residue was purified by flash chromatography on silica gel eluting with dichloromethane/methanol (96:4) to afford 4-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-N,N-bis(4-methoxybenzyl)-6-methyl-5-(trifluoromethyl)pyridin-2-amine (210.0 mg, 0.22 mmol, 72.3% yield) as a white solid. LC-MS: (ESI, m/z): 942.4 [M+H]⁺.

### Step 2: 4-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-6-methyl-5-(trifluoromethyl)pyridin-2-amine

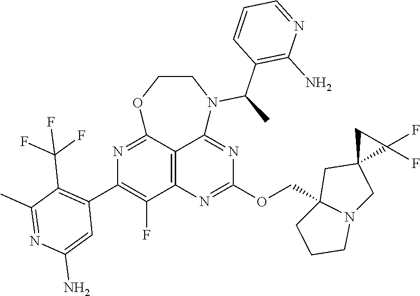 [SMILES: Cc1nc(N)cc(-c2nc3c4c(nc(OC[C@@]56CCCN5C[C@]5(CC5(F)F)C6)nc4c2F)N([C@H](C)c2cccnc2N)CCO3)c1C(F)(F)F]

A solution of 4-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-N,N-bis(4-methoxybenzyl)-6-methyl-5-(trifluoromethyl)pyridin-2-amine (210.0 mg, 0.22 mmol) in trifluoromethanesulfonic acid (0.3 mL) and trifluoroacetic acid (3 mL) was stirred at 25° C. for 0.5 hours. After completion, the reaction mixture was concentrated under vacuum and diluted with dichloromethane, adjusted to pH>7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (96:4) to afford product. The product was further purified by Prep-HPLC with the following conditions: Column: XSelect CSH Fluoro Phenyl 30*150 mm, 5 μm; Mobile Phase A: Water (10 mmol/L NH₄HCO₃), Mobile Phase B: ACN; Flow rate: 60 mL/min; Gradient: 17% B to 42% B in 9 min; Wave Length: 254 nm/220 nm; RT1(min): 7.9 to afford 4-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-6-methyl-5-(trifluoromethyl)pyridin-2-amine (40.2 mg, 0.06 mmol, 25.7% yield) as a white solid. LC-MS: (ESI, m/z): 702.2 [M+H]⁺.

Example 615: ¹H NMR (300 MHz, Methanol-d₄) δ7.98 (dd, J=5.1, 1.6 Hz, 1H), 7.78 (d, J=7.5 Hz, 1H), 6.80 (dd, J=7.5, 5.1Hz, 1H), 6.64 (q, J=6.9 Hz, 1H), 6.35 (s, 1H), 4.48 (dd, J=12.8, 6.3 Hz, 1H), 4.43 (s, 2H), 4.35 (dd, J=12.8, 6.5 Hz, 1H), 3.77 (dd, J=16.1, 6.5 Hz, 1H), 3.56 (dd, J=16.0, 6.3 Hz, 1H), 3.28 (d, J=6.9 Hz, 1H), 3.25-3.13 (m, 1H), 2.86 (d, J=12.2 Hz, 1H), 2.78-2.65 (m, 1H), 2.60-2.49 (m, 3H), 2.37-2.24 (m, 1H), 2.18 (dd, J=11.2, 6.2 Hz, 1H), 2.05 (d, J=13.5 Hz, 1H), 1.99-1.87 (m, 2H), 1.86-1.76 (m, 1H), 1.68 (d, J=6.9 Hz, 3H), 1.55-1.36 (m, 2H). LC-MS: (ESI, m/z): 702.2 [M+H]⁺.

### Example 616: 5-((R)-1-(5-(5-amino-4-fluoro-3-methyl-2-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyrimidin-4-amine

### Synthetic Route

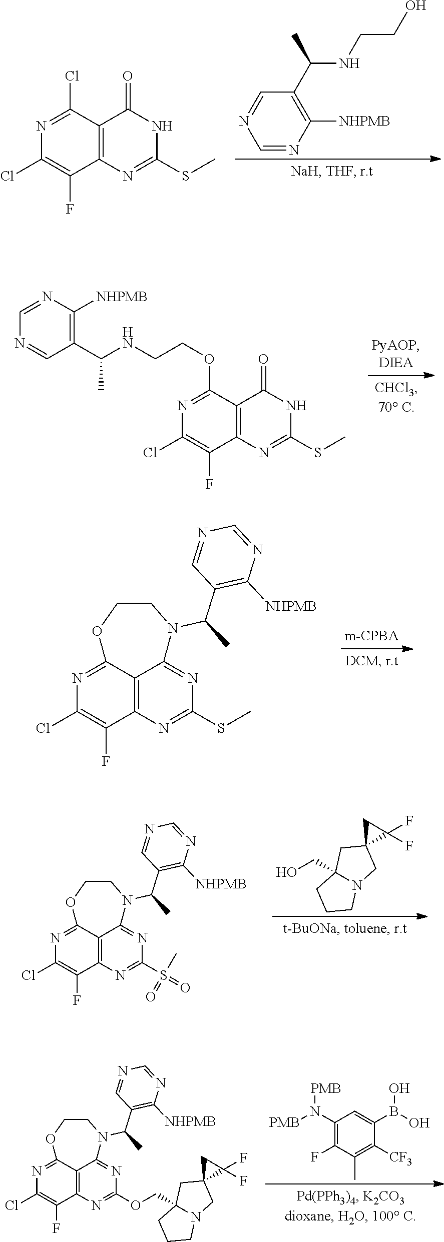

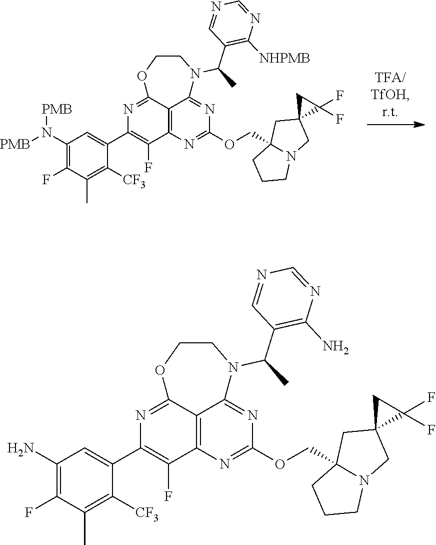

### Step 1: (R)-7-chloro-8-fluoro-5-(2-((1-(4-((4-methoxybenzyl)amino)pyrimidin-5-yl)ethyl)amino)ethoxy)-2-(methylthio)pyrido[4,3-d]pyrimidin-4(3H)-one

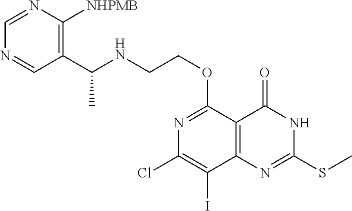

A solution of (R)-2-((1-(4-((4-methoxybenzyl)amino)pyrimidin-5-yl)ethyl)amino)ethan-1-ol (2.60 g, 8.60 mmol) in tetrahydrofuran (40 mL) was added sodium hydride (1.14 g, 28.56 mmol, 60% dispersion in mineral oil) and stirred at 0° C. for 15 minutes. Then the mixture was transferred into a solution of 5,7-dichloro-8-fluoro-2-methylsulfanyl-3H-pyrido[4,3-d]pyrimidin-4-one (2.00 g, 7.14 mmol) in tetrahydrofuran (20 mL) and stirred at 25° C. for 2 hours. After completion, the reaction was quenched with saturated ammonium chloride solution. The reaction mixture was extracted with ethyl acetate and the organic layers were combined. The organic layer was dried over anhydrous sodium sulfate and concentrated under vacuum. The residue was purified by flash chromatography on silica gel eluting with dichloromethane/methanol (10:1) to afford (R)-7-chloro-8-fluoro-5-(2-((1-(4-((4-methoxybenzyl)amino)pyrimidin-5-yl)ethyl)amino)ethoxy)-2-(methylthio)pyrido[4,3-d]pyrimidin-4(3H)-one (3.21 g, 5.88 mmol, 82% yield) as a yellow solid. LC-MS: (ESI, m/z): 546.1 [M+H]⁺

### Step 2: (R)-5-(1-(5-chloro-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrimidin-4-amine

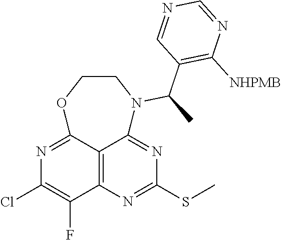

A mixture of (R)-7-chloro-8-fluoro-5-(2-((1-(4-((4-methoxybenzyl)amino)pyrimidin-5-yl)ethyl)amino)ethoxy)-2-(methylthio)pyrido[4,3-d]pyrimidin-4(3H)-one (2.00 g, 3.60 mmol), 7-azabenzotriazol-1-yloxy)tripyrrolidinophosphonium hexafluorophosphate (2.90 g, 5.40 mmol) and N,N-diisopropylethylamine (2.40 g, 18.30 mmol) in chloroform (30 mL) was stirred at 70° C. for 3 hours. After completion, the reaction was diluted with dichloromethane and washed with water. The organic layers were combined, washed with brine, dried over anhydrous sodium sulfate and concentrated. The residue was purified by flash chromatography on silica gel eluting with dichloromethane/methanol (30:1) to afford crude product. The resulting residue was purified by reverse phase chromatography (acetonitrile/0.1% ammonium bicarbonate in water) to afford (R)-5-(1-(5-chloro-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrimidin-4-amine (700.0 mg, 1.30 mmol, 36% yield) as a yellow solid. LC-MS: (ESI, m/z): 528.1 [M+H]⁺

### Step 3: (R)-5-(1-(5-chloro-4-fluoro-2-(methylsulfonyl)-8,9-dihydro-10H-7-oxa-1,3,6,10-tetraazacyclohepta[de]naphthalen-10-yl)ethyl)-N-(4-methoxybenzyl)pyrimidin-4-amine

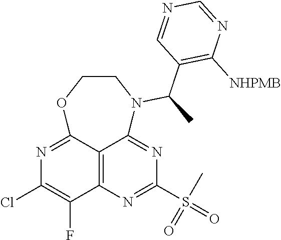

A solution of (R)-5-(1-(5-chloro-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrimidin-4-amine (210.0 mg, 0.4 mmol) and 3-chloroperoxybenzoic acid (171.0 mg, 0.90 mmol) in dichloromethane (2 mL) was stirred at 25° C. for 1 hour. After completion, the reaction was quenched with saturated sodium sulfite solution. The resulting solution was extracted with dichloromethane. The organic layer was washed with brine, dried over anhydrous sodium sulfate and concentrated under vacuum. The residue was purified by flash chromatography on silica gel eluting with dichloromethane/methanol (10:1) to afford (R)-5-(1-(5-Chloro-4-fluoro-2-(methylsulfonyl)-8,9-dihydro-10H-7-oxa-1,3,6,10-tetraazacyclohepta[de]naphthalen-10-yl)ethyl)-N-(4-methoxybenzyl)pyrimidin-4-amine (150.0 mg, 0.2 mmol, 67% yield) as a yellow solid. LC-MS: (ESI, m/z): 560.1 [M+H]⁺

### Step 4: 5-((R)-1-(5-chloro-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N-(4-methoxybenzyl)pyrimidin-4-amine

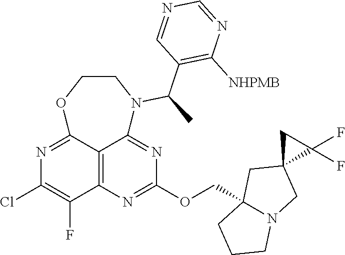

To a solution of ((1R,7a'S)-2,2-difluorodihydro-1′H,3′H-spiro[cyclopropane-1,2′-pyrrolizin]-7a′(5′H)-yl)methanol (94.3 mg, 0.40 mmol) in toluene (2.0 mL) was added sodium tert-butoxide (111.4 mg, 1.01 mmol), the mixture was stirred at 0° C. for 15 minutes. Then (R)-5-(1-(5-chloro-4-fluoro-2-(methylsulfonyl)-8,9-dihydro-10H-7-oxa-1,3,6,10-tetraazacyclohepta[de]naphthalen-10-yl)ethyl)-N-(4-methoxybenzyl)pyrimidin-4-amine (130.0 mg, 0.20 mmol) was added and stirred at 25° C. for 2 hours. After completion, the reaction mixture was concentrated under vacuum, diluted with ethyl acetate, washed with water and the organic layers were combined. The organic layer was dried over anhydrous sodium sulfate and concentrated under vacuum. The residue was purified by flash chromatography on silica gel eluting with methanol/dichloromethane (1:8) to afford 5-((R)-1-(5-chloro-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N-(4-methoxybenzyl)pyrimidin-4-amine (70.0 mg, 0.1 mmol, 44% yield) as a white solid. LC-MS: (ESI, m/z): 683.2 [M+H]⁺

### Step 5: 5-((R)-1-(5-(5-(bis(4-methoxybenzyl)amino)-4-fluoro-3-methyl-2-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N-(4-methoxybenzyl)pyrimidin-4-amine

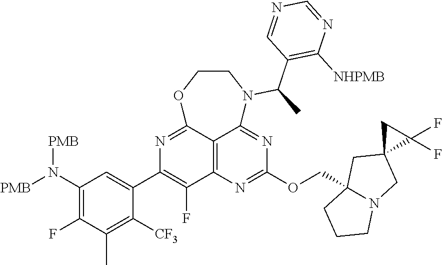

Under nitrogen, a solution of 5-((R)-1-(5-chloro-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N-(4-methoxybenzyl)pyrimidin-4-amine (77.0 mg, 0.1 mmol), [5-[bis[(4-methoxyphenyl)methyl]amino]-4-fluoro-3-methyl-2-(trifluoromethyl)phenyl]boronic acid (80.6 mg, 0.1 mmol), tetrakis(triphenylphosphine)palladium (13.0 mg, 0.01 mmol) and potassium carbonate (46.6 mg, 0.30 mmol) in 1,4-dixone (0.5 mL) and water (0.1 mL) was stirred at 100° C. for 2 hours. After completion, the reaction mixture was diluted with ethyl acetate, washed with water, dried over anhydrous sodium sulfate and concentrated under vacuum. The residue was purified by flash chromatography on silica gel eluting with dichloromethane/methanol (10:1) to afford 5-((R)-1-(5-(5-(bis(4-methoxybenzyl)amino)-4-fluoro-3-methyl-2-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N-(4-methoxybenzyl)pyrimidin-4-amine (90.0 mg, 0.08 mmol, 74% yield). LC-MS: (ESI, m/z): 1080.4 [M+H]⁺

### Step 6: 5-((R)-1-(5-(5-amino-4-fluoro-3-methyl-2-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyrimidin-4-amine

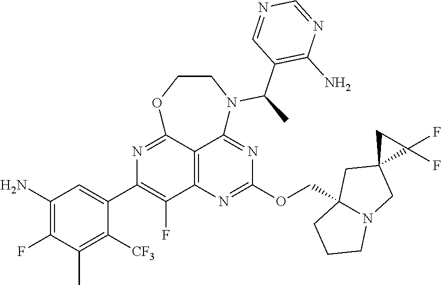 [SMILES: Cc1c(F)c(N)cc(-c2nc3c4c(nc(OC[C@@]56CCCN5C[C@]5(CC5(F)F)C6)nc4c2F)N([C@H](C)c2cncnc2N)CCO3)c1C(F)(F)F]

A solution of 5-((R)-1-(5-(5-(bis(4-methoxybenzyl)amino)-4-fluoro-3-methyl-2-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N-(4-methoxybenzyl)pyrimidin-4-amine (80.0 mg, 0.07 mmol) in trifluoroacetic acid (1.0 mL) and trifluoromethanesulfonic acid (0.1 mL) was stirred at 25° C. for 8 hours. After completion, the reaction mixture was concentrated under reduced pressure. The reaction mixture was diluted with dichloromethane and adjusted to pH=7 with saturated sodium bicarbonate solution. The resulting solution was extracted with dichloromethane and the organic layers were combined. The organic layer was dried over anhydrous sodium sulfate and concentrated under vacuum. The residue was purified by flash chromatography on silica gel eluting with dichloromethane/methanol (10:1) to afford crude product. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min; Wave Length: 254 nm/220 nm to afford 5-((R)-1-(5-(5-amino-4-fluoro-3-methyl-2-(trifluoromethyl)phenyl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyrimidin-4-amine(20.8 mg, 0.02 mmol, 39% yield) as a white solid. LC-MS: (ESI, m/z): 720.4 [M+H]⁺

Example 616: ¹H NMR (300 MHz, DMSO-d₆, ppm) δ 8.39 (s, 1H), 8.30 (s, 1H), 6.76 (s, 2H), 6.55 (d, J=8.7 Hz, 1H), 6.28 (q, J=6.7 Hz, 1H), 6.02 (s, 2H), 4.55-4.41 (m, 1H), 4.40-4.15 (m, 3H), 3.81 (dd, J=16.0, 6.3 Hz, 1H), 3.49 (dd, J=15.8, 6.3 Hz, 1H), 3.18-2.96 (m, 2H), 2.72 (d, J=11.8 Hz, 1H), 2.61-2.51 (m, 1H), 2.39-2.26 (m, 3H), 2.14-1.94 (m, 2H), 1.92-1.70 (m, 3H), 1.68-1.40 (m, 6H). LCMS (ESI, m/z): 720.4 [M+H]⁺.

### Example 617: 5-((R)-1-(5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrimidin-4-amine

### Synthetic Route

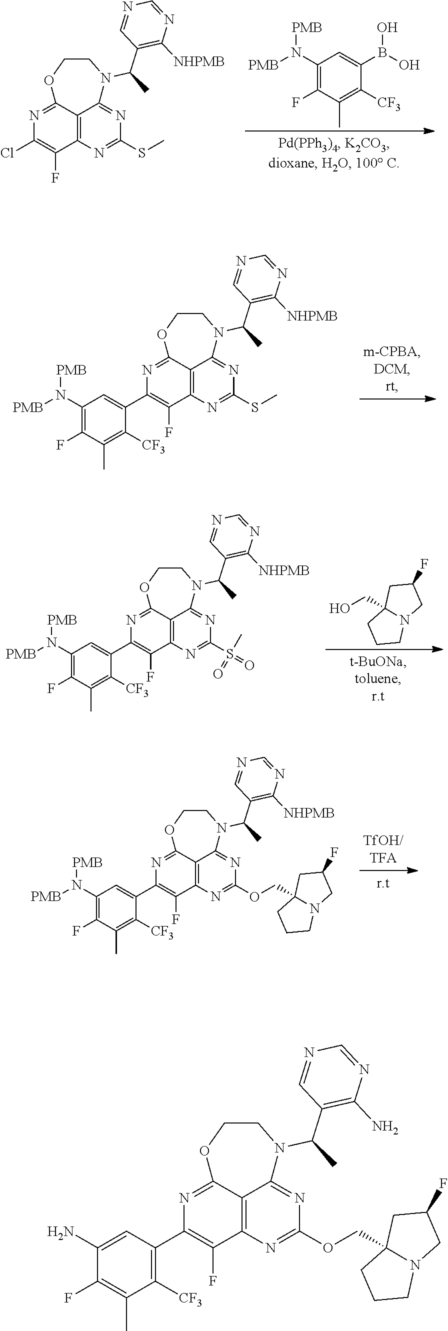

### Step 1: (R)-5-(1-(5-(5-(bis(4-methoxybenzyl)amino)-4-fluoro-3-methyl-2-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrimidin-4-amine

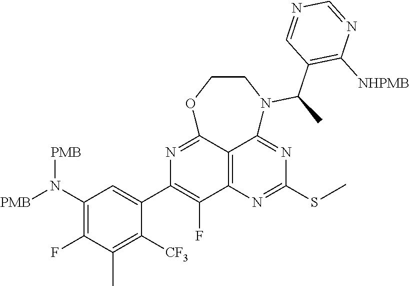

Under nitrogen, a solution of (R)-5-(1-(5-chloro-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrimidin-4-amine (300.0 mg, 0.59 mmol), (5-(bis(4-methoxybenzyl)amino)-4-fluoro-3-methyl-2-(trifluoromethyl)phenyl)boronic acid (406.8 mg, 0.85 mmol), tetrakis(triphenylphosphine)palladium (65.6 mg, 0.06 mmol) and potassium carbonate (235.2 mg, 1.7 mmol) in 1,4-dioxane(3 mL) and water (0.6 mL) was stirred at 100° C. for 10 hours. After completion, the reaction mixture was diluted with water, extracted with ethyl acetate and the organic layers were combined. The organic layer was dried over anhydrous sodium sulfate and concentrated under vacuum. The residue was purified by flash chromatography on silica gel eluting with methanol/dichloromethane (1:8) to afford (R)-5-(1-(5-(5-(bis(4-methoxybenzyl)amino)-4-fluoro-3-methyl-2-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrimidin-4-amine (500.0 mg, 0.50 mmol, 95% yield) as a yellow solid. LC-MS: (ESI, m/z): 925.3 [M+H]⁺

### Step 2: (R)-5-(1-(5-(5-(bis(4-methoxybenzyl)amino)-4-fluoro-3-methyl-2-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N-(4-methoxybenzyl)pyrimidin-4-amine

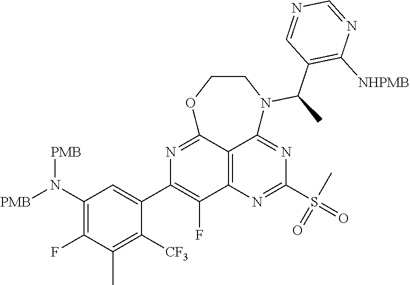

A solution of (R)-5-(1-(5-(5-(bis(4-methoxybenzyl)amino)-4-fluoro-3-methyl-2-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrimidin-4-amine (370.0 mg, 0.40 mmol) and 3-chloroperoxybenzoic acid (172.5 mg, 1.00 mmol) in dichloromethane (5 mL) was stirred at 25° C. for 1 hour. After completion, the reaction was quenched with saturated sodium sulfite solution. The resulting solution was extracted with dichloromethane. The organic layer was washed with brine, dried over anhydrous sodium sulfate. The solvent was concentrated under vacuum. The residue was purified by flash chromatography on silica gel eluting with methanol/dichloromethane (1:8) to afford (R)-5-(1-(5-(5-(bis(4-methoxybenzyl)amino)-4-fluoro-3-methyl-2-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N-(4-methoxybenzyl)pyrimidin-4-amine (180.0 mg, 0.18 mmol, 47% yield) as a yellow solid. LC-MS: (ESI, m/z): 957.3 [M+H]⁺

### Step 3: 5-((R)-1-(5-(5-(bis(4-methoxybenzyl)amino)-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrimidin-4-amine

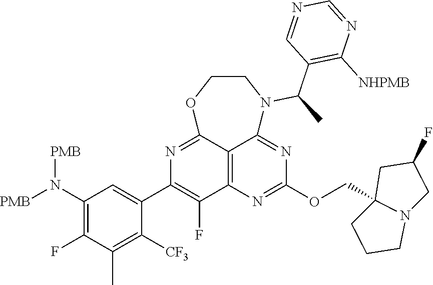

To a mixture of [(2R,8S)-2-fluoro-1,2,3,5,6,7-hexahydropyrrolizin-8-yl]methanol (99.8 mg, 0.6 mmol) in toluene (3 mL) was added sodium tert-butoxide (100.3 mg, 1.00 mmol), the mixture was stirred at 0° C. for 15 minutes. Then the reaction mixture was transferred into a solution of (R)-5-(1-(5-(5-(bis(4-methoxybenzyl)amino)-4-fluoro-3-methyl-2-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N-(4-methoxybenzyl)pyrimidin-4-amine (180.0 mg, 0.18 mmol) in toluene (2 mL) and stirred at 25° C. for 1 hour. After completion, the reaction was quenched with saturated ammonium chloride solution. The reaction mixture was diluted with water, extracted with ethyl acetate and the organic layers were combined. The organic layer was dried over anhydrous sodium sulfate and concentrated under vacuum. The residue was purified by flash chromatography on silica gel eluting with methanol/dichloromethane (1:20) to afford 5-((R)-1-(5-(5-(Bis(4-methoxybenzyl)amino)-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrimidin-4-amine (150.0 mg, 0.1 mmol, 69% yield) as a yellow solid. LC-MS: (ESI, m/z): 1036.4 [M+H]⁺

### Step 4: 5-((R)-1-(5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrimidin-4-amine

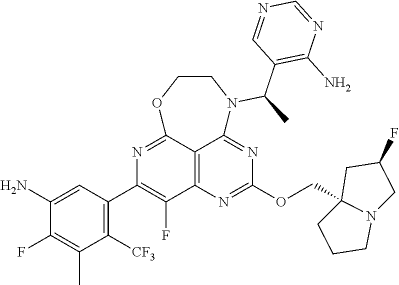 [SMILES: Cc1c(F)c(N)cc(-c2nc3c4c(nc(OC[C@@]56CCCN5C[C@H](F)C6)nc4c2F)N([C@H](C)c2cncnc2N)CCO3)c1C(F)(F)F]

A solution of 5-((R)-1-(5-(5-(bis(4-methoxybenzyl)amino)-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N-(4-methoxybenzyl)pyrimidin-4-amine (100.0 mg, 0.10 mmol) in trifluoroacetic acid (2 mL) and trifluoromethanesulfonic acid (0.2 mL) was stirred at 25° C. for 10 hours. After completion, the solvent was concentrated under reduced pressure. The reaction mixture was diluted with dichloromethane and adjusted to pH=7 with saturated sodium bicarbonate solution. The resulting solution was extracted with dichloromethane and the organic layers were combined. The organic layer was dried over anhydrous sodium sulfate and concentrated under vacuum. The residue was purified by flash chromatography on silica gel eluting with dichloromethane/methanol (10:1) to afford crude product. The crude product was purified by Prep-HPLC with the following conditions: Column: XBridge Prep OBD C18 Column, 30*150 mm, Sum; Mobile Phase A: Water(10 mmol/L NH₄HCO₃), Mobile Phase B: ACN; Flow rate: 60 mL/min; Gradient: 17% B to 42% B in 9 min; Wave Length: 254 nm/220 nm to afford 5-((R)-1-(5-(5-amino-4-fluoro-3-methyl-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrimidin-4-amine (5.6 mg, 0.008 mmol, 9% yield) as a white solid. LCMS (ESI, m/z): 676.3 [M+H]⁺

Example 617: ¹H NMR (300 MHz, Methanol-d₄, ppm) δ 8.41 (s, 1H), 8.34 (s, 1H), 6.66-6.51 (m, 2H), 5.33 (d, J=53.7 Hz, 1H), 4.66-4.35 (m, 2H), 4.31 (s, 2H), 3.93-3.79 (m, 1H), 3.71-3.56 (m, 1H), 3.31-3.14 (m, 3H), 3.12-2.95 (m, 1H), 2.46-2.33 (m, 3H), 2.31-2.08 (m, 3H), 2.08-1.85 (m, 3H), 1.70 (d, J=6.9 Hz, 3H). LCMS (ESI, m/z): 676.3 [M+H]⁺.

### Example 618: 1-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)isoquinolin-3-amine

### Synthetic Route

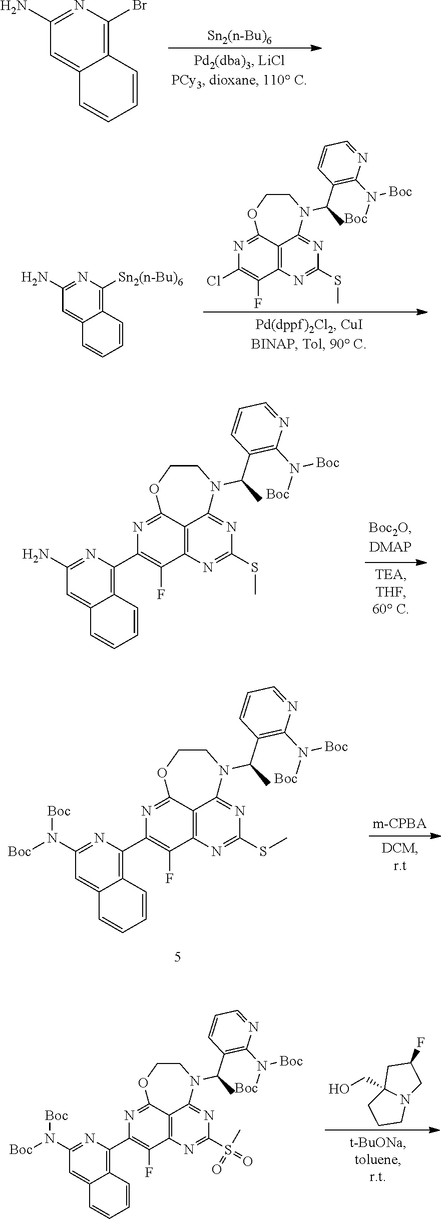

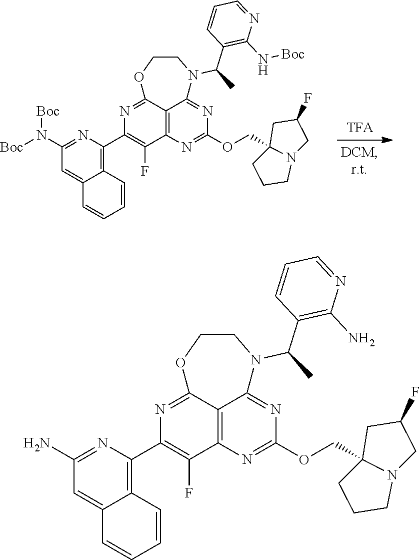 [SMILES: C[C@H](c1cccnc1NC(=O)OC(C)(C)C)N1CCOc2nc(-c3nc(N(C(=O)OC(C)(C)C)C(=O)OC(C)(C)C)cc4ccccc34)c(F)c3nc(OC[C@@]45CCCN4C[C@H](F)C5)nc1c23 ; C[C@H](c1cccnc1N)N1CCOc2nc(-c3nc(N)cc4ccccc34)c(F)c3nc(OC[C@@]45CCCN4C[C@H](F)C5)nc1c23]

### Step 1: 1-tributylstannylisoquinolin-3-amine

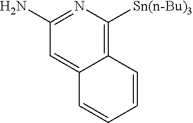

Under nitrogen, a solution of 1-bromoisoquinolin-3-amine (400.0 mg, 1.7 mmol), hexabutylditin (1.14 g, 1.97 mmol), lithiumchloride (389.1 mg, 8.9 mmol) and tricyclohexylphosphine (100.5 mg, 0.36 mmol) in 1,4-dioxane (8 mL) was stirred at 110° C. for 4 hours. After completion, the reaction was quenched with saturated potassium fluoride solution, extracted with ethyl acetate and washed with brine. The organic layers were combined, concentrated under vacuum and purified by flash chromatography on silica gel eluting with ethyl acetate/petroleum ether (3:1) to afford 1-tributylstannylisoquinolin-3-amine (290 mg, 0.32 mmol, 18.3% yield) as a light yellow solid. LC-MS: (ESI, m/z): 435.2 [M+H]⁺

### Step 2: tert-butyl N-[3-[(1R)-1-[7-(3-amino-1-isoquinolyl)-6-fluoro-3-methylsulfanyl-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate

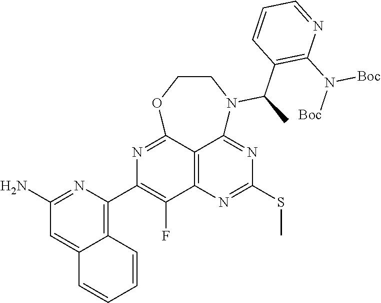 [SMILES: CSc1nc2c3c(nc(-c4nc(N)cc5ccccc45)c(F)c3n1)OCCN2[C@H](C)c1cccnc1N(C(=O)OC(C)(C)C)C(=O)OC(C)(C)C]

Under nitrogen, a mixture of 1-tributylstannylisoquinolin-3-amine (231.9 mg, 0.5 mmol) and tert-butyl N-tert-butoxycarbonyl-N-[3-[(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl]-2-pyridyl]carbamate (130.0 mg, 0.2 mmol), [1,1′-bis(diphenylphosphino)ferrocene]dichloropalladium(II) (16.4 mg, 0.02 mmol), cuprous iodide (11.3 mg, 0.06 mmol) and 1.1′-binaphthyl-2.2′-diphemyl phosphine (26.6 mg, 0.04 mmol) in toluene (3 mL) was stirred at 90° C. for 4 hours. After completion, the solvent was concentrated under vacuum. The residue was purified by flash chromatography on silica gel eluting with dichloromethane/ethyl acetate (5:1) to afford tert-butyl N-[3-[(1R)-1-[7-(3-amino-1-isoquinolyl)-6-fluoro-3-methylsulfanyl-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (82.0 mg, 0.11 mmol, 53.6% yield) as a white solid. LC-MS: (ESI, m/z): 715.3 [M+H]⁺

### Step 3: tert-butyl N-[3-[(1R)-1-[7-[3-[bis(tert-butoxycarbonyl)amino]-1-isoquinolyl]-6-fluoro-3-methylsulfanyl-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate

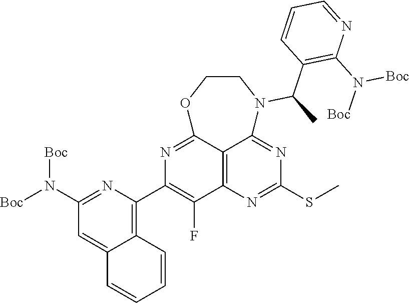 [SMILES: CSc1nc2c3c(nc(-c4nc(N(C(=O)OC(C)(C)C)C(=O)OC(C)(C)C)cc5ccccc45)c(F)c3n1)OCCN2[C@H](C)c1cccnc1N(C(=O)OC(C)(C)C)C(=O)OC(C)(C)C]

A solution of tert-butyl N-[3-[(1R)-1-[7-(3-amino-1-isoquinolyl)-6-fluoro-3-methylsulfanyl-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (72.0 mg, 0.1 mmol) and di-tert-butyl dicarbonate (87.8 mg, 0.4 mmol), 4-dimethylaminopyridine (2.2 mg, 0.02 mmol), triethylamine (61.0 mg, 0.6 mmol) in tetrahydrofuran (1 mL) was stirred at 60° C. for 1 hour. After completion, the solvent was concentrated under vacuum. The residue was purified by flash chromatography on silica gel eluting with ethyl acetate/dichloromethane (1:5) to afford tert-butyl N-[3-[(1R)-1-[7-[3-[bis(tert-butoxycarbonyl)amino]-1-isoquinolyl]-6-fluoro-3-methylsulfanyl-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (60.0 mg, 0.06 mmol, 61.8% yield) as a white solid. LC-MS: (ESI, m/z): 915.4 [M+H]⁺

### Step 4: tert-butyl (R)-(3-(1-(5-(3-(bis(tert-butoxycarbonyl)amino)isoquinolin-1-yl)-4-fluoro-2-(methylsulfonyl)-8,9-dihydro-10H-7-oxa-1,3,6,10-tetraazacyclohepta[de]naphthalen-10-yl)ethyl)pyridin-2-yl)(tert-butoxycarbonyl)carbamate

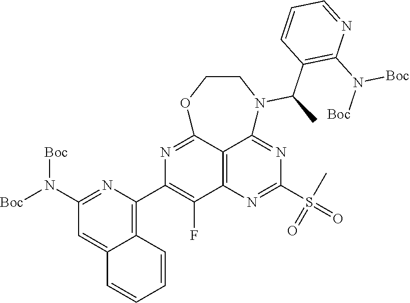 [SMILES: C[C@H](c1cccnc1N(C(=O)OC(C)(C)C)C(=O)OC(C)(C)C)N1CCOc2nc(-c3nc(N(C(=O)OC(C)(C)C)C(=O)OC(C)(C)C)cc4ccccc34)c(F)c3nc(S(C)(=O)=O)nc1c23]

To a solution of tert-butyl N-[3-[(1R)-1-[7-[3-[bis(tert-butoxycarbonyl)amino]-1-isoquinolyl]-6-fluoro-3-methylsulfanyl-10-oxa-2,4,8,13-tetrazatricyclo[7.4.1.05, 14]tetradeca-1,3,5(14),6,8-pentaen-13-yl]ethyl]-2-pyridyl]-N-tert-butoxycarbonyl-carbamate (60.0 mg, 0.07 mmol) in dichloromethane (2 mL) was added 3-chloroperoxybenzoic acid (33.8 mg, 0.2 mmol), the mixture was stirred at 25° C. for 1 hour. After completion, the reaction was quenched with saturated sodium sulfite solution. The resulting solution was diluted with water and extracted with dichloromethane. The organic layers were combined, washed with brine, dried over anhydrous sodium sulfate and concentrated. The residue was purified by flash chromatography on silica gel eluting with dichloromethane/ethyl acetate (6:1) to afford tert-butyl (R)-(3-(1-(5-(3-(bis(tert-butoxycarbonyl)amino)isoquinolin-1-yl)-4-fluoro-2-(methylsulfonyl)-8,9-dihydro-10H-7-oxa-1,3,6,10-tetraazacyclohepta[de]naphthalen-10-yl)ethyl)pyridin-2-yl)(tert-butoxycarbonyl)carbamate (57.0 mg, 0.05 mmol, 78% yield) as a yellow solid. LC-MS: (ESI, m/z): 947.4 [M+H]⁺

### Step 5: tert-butyl (tert-butoxycarbonyl)(1-(10-((R)-1-(2-((tert-butoxycarbonyl)amino)pyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)isoquinolin-3-yl)carbamate

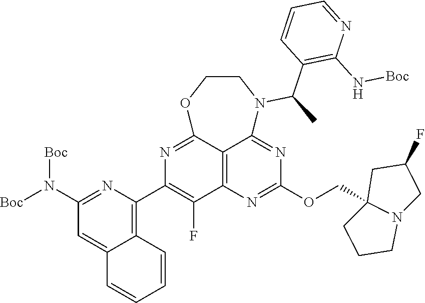 [SMILES: C[C@H](c1cccnc1NC(=O)OC(C)(C)C)N1CCOc2nc(-c3nc(N(C(=O)OC(C)(C)C)C(=O)OC(C)(C)C)cc4ccccc34)c(F)c3nc(OC[C@@]45CCCN4C[C@H](F)C5)nc1c23]

To a solution of ((2R,7aS)-2-fluorotetrahydro-1H-pyrrolizin-7a(5H)-yl)methanol (28.7 mg, 0.1 mmol) in toluene (2 mL) was added sodium tert-butoxide (17.3 mg, 0.18 mmol), the mixture was stirred for 10 minutes at 0° C. Then tert-butyl (R)-(3-(1-(5-(3-(bis(tert-butoxycarbonyl)amino)isoquinolin-1-yl)-4-fluoro-2-(methylsulfonyl)-8,9-dihydro-10H-7-oxa-1,3,6,10-tetraazacyclohepta[de]naphthalen-10-yl)ethyl)pyridin-2-yl)(tert-butoxycarbonyl)carbamate (57.0 mg, 0.06 mmol) was added and stirred at 25° C. for 1 hour. After completion, the solvent was concentrated under vacuum. The residue was purified by flash chromatography on silica gel eluting with dichloromethane/methanol (12/1) to afford tert-butyl (tert-butoxycarbonyl)(1-(10-((R)-1-(2-((tert-butoxycarbonyl)amino)pyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)isoquinolin-3-yl)carbamate (22.0 mg, 0.02 mmol, 32.8% yield) as a white solid. LC-MS: (ESI, m/z): 926.4 [M+H]⁺

### Step 6: 1-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)isoquinolin-3-amine

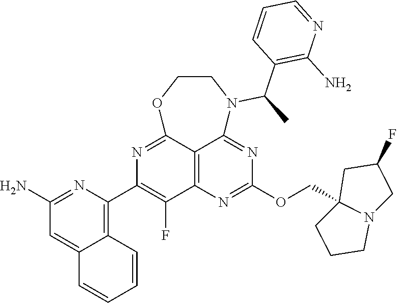 [SMILES: C[C@H](c1cccnc1N)N1CCOc2nc(-c3nc(N)cc4ccccc34)c(F)c3nc(OC[C@@]45CCCN4C[C@H](F)C5)nc1c23]

A solution of tert-butyl (tert-butoxycarbonyl)(1-(10-((R)-1-(2-((tert-butoxycarbonyl)amino)pyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)isoquinolin-3-yl)carbamate (22.0 mg, 0.02 mmol) and trifluoroacetic acid (0.2 mL) in dichloromethane (1 mL) was stirred at 25° C. for 1 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min; Wave Length: 254 nm/220 nm; RT1(min): 8.3 to afford 1-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)isoquinolin-3-amine (7.7 mg, 0.01 mmol, 51.6% yield) as a yellow solid. LC-MS: (ESI, m/z): 626.3 [M+H]⁺

Example 618: ¹H NMR (300 MHz, Methanol-d₄, ppm) δ 8.00-7.91 (m, 1H), 7.76 (d, J=7.6 Hz, 1H), 7.62 (d, J=8.4 Hz, 1H), 7.59-7.44 (m, 2H), 7.23-7.09 (m, 1H), 6.93 (s, 1H), 6.78 (dd, J=7.5, 5.2 Hz, 1H), 6.61 (q, J=9.4, 8.4 Hz, 1H), 5.30 (d, J=54.0 Hz, 1H), 4.52-4.36 (m, 2H), 4.33 (s, 2H), 3.78 (dd, J=15.9, 6.4 Hz, 1H), 3.56 (dd, J=15.9, 6.2 Hz, 1H), 3.26-3.07 (m, 3H), 3.07-2.91 (m, 1H), 2.47-2.07 (m, 3H), 2.07-1.81 (m, 3H), 1.66 (d, J=6.9 Hz, 3H). LC-MS: (ESI, m/z): 626.3 [M+H]⁺

### Example 620: 6-(10-((R)-1-(2-aminopyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-4-methyl-5-(trifluoromethyl)pyridin-2-amine

### Synthetic Route

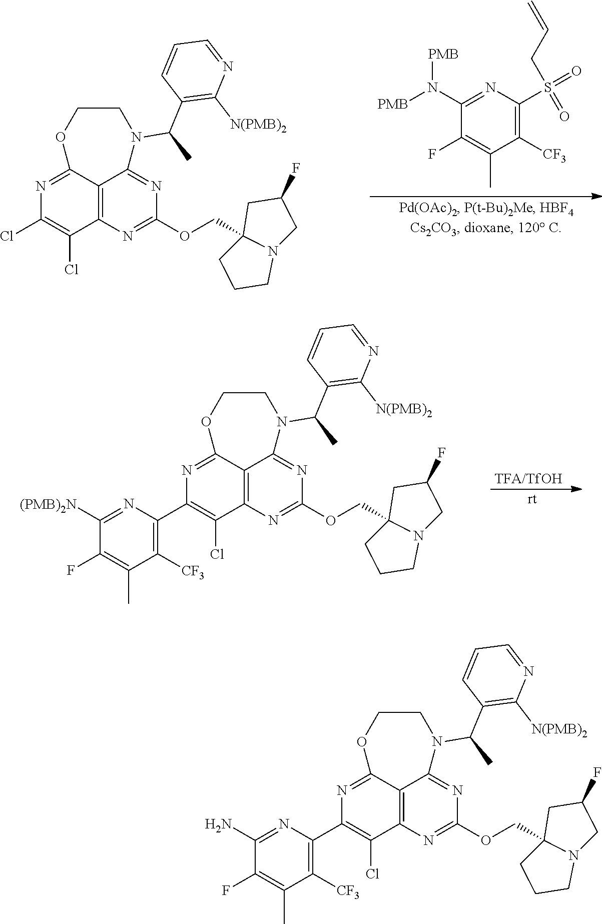

### Step 1: 6-(10-((R)-1-(2-(bis(4-methoxybenzyl)amino)pyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-N,N-bis(4-methoxybenzyl)-4-methyl-5-(trifluoromethyl)pyridin-2-amine

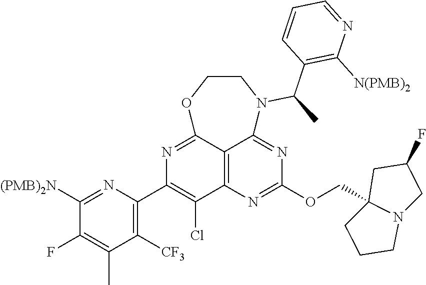

Under nitrogen, a mixture of 3-((R)-1-(4,5-dichloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (90.0 mg, 0.10 mmol), 6-allylsulfonyl-3-fluoro-N,N-bis[(4-methoxyphenyl)methyl]-4-methyl-5-(trifluoromethyl)pyridin-2-amine (111.0 mg, 0.20 mmol), palladium (II) acetate (3.0 mg, 0.01 mmol), di-tert-butyl(methyl)phosphonium tetrafluoroborate (7.0 mg, 0.02 mmol) and cesium carbonate (136.0 mg, 0.40 mmol) in 1,4-dioxane (3.0 mL) was stirred at 120° C. for 2 hours. After complete, the reaction mixture was diluted with water and extracted with dichloromethane. The combined organic layers were washed with brine, dried over anhydrous sodium sulfate and concentrated in vacuum. The residue was purified by flash chromatography on silica gel eluting with dichloromethane/methanol (15:1) to afford 6-(10-((R)-1-(2-(bis(4-methoxybenzyl)amino)pyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-N,N-bis(4-methoxybenzyl)-4-methyl-5-(trifluoromethyl)pyridin-2-amine (95.0 mg, 75% purity, 0.06 mmol, 61% yield) as a light yellow oil. LC-MS: (ESI, m/z): 1172.2 [M+H]⁺

### Step 2: 6-(10-((R)-1-(2-aminopyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-4-methyl-5-(trifluoromethyl)pyridin-2-amine

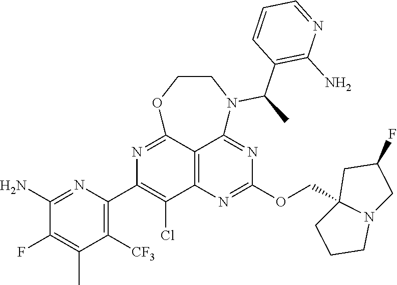 [SMILES: Cc1c(F)c(N)nc(-c2nc3c4c(nc(OC[C@@]56CCCN5C[C@H](F)C6)nc4c2Cl)N([C@H](C)c2cccnc2N)CCO3)c1C(F)(F)F]

A solution of 6-(10-((R)-1-(2-(bis(4-methoxybenzyl)amino)pyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-N,N-bis(4-methoxybenzyl)-4-methyl-5-(trifluoromethyl)pyridin-2-amine (90.0 mg, 0.04 mmol) in trifluoroacetic acid (3 mL) and trifluoromethanesulfonic acid (0.3 mL) was stirred at 25° C. for 1 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min; Wave Length: 254 nm/220 nm; RT1(min): 9.6 to afford 6-(10-((R)-1-(2-aminopyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-4-methyl-5-(trifluoromethyl)pyridin-2-amine (2.0 mg, 0.03 mmol, 4% yield) as a white solid. LC-MS: (ESI, m/z): 692.3 [M+H]⁺

Example 620: ¹H NMR (400 MHz, DMSO-d₆, ppm) δ 7.97 (d, J=4.2 Hz, 1H), 7.62 (d, J=7.6 Hz, 1H), 7.10 (s, 2H), 6.67 (dd, J=7.2, 4.9, 2.1Hz, 1H), 6.33 (q, J=6.8 Hz, 1H), 5.68 (d, J=5.9 Hz, 2H), 5.29 (d, J=54.4 Hz, 1H), 4.48-4.26 (m, 2H), 4.22-4.07 (m, 2H), 3.78-3.64 (m, 1H), 3.19-3.01 (m, 3H), 2.87-2.77 (m, 1H), 2.46-2.18 (m, 4H), 2.19-1.98 (m, 3H), 1.93-1.70 (m, 3H), 1.56 (t, J=7.5 Hz, 3H). LCMS (ESI, m/z): 692.3 [M+H]⁺.

### Example 621: 3-((1R)-1-(5-(3-amino-2,4-difluoro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Synthetic Route

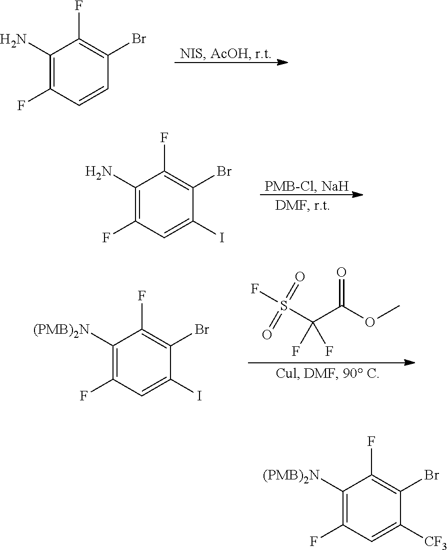

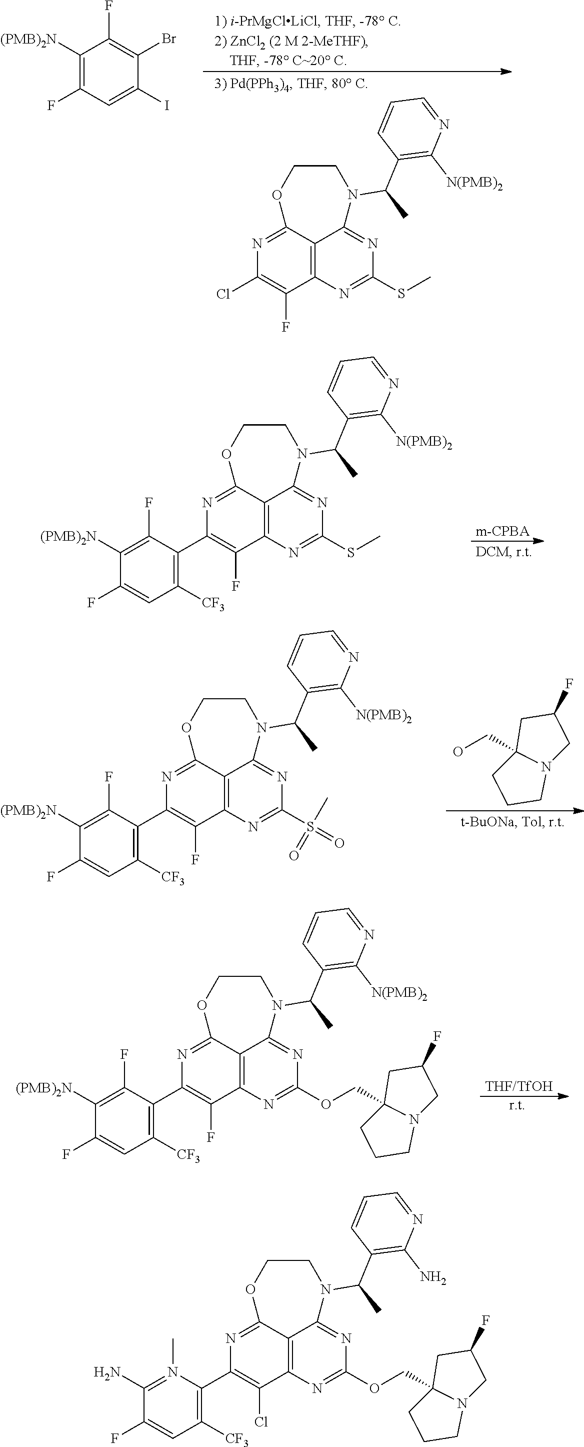

### Step 1: 3-bromo-2,6-difluoro-4-iodoaniline

A solution of 3-bromo-2,6-difluoroaniline (1.00 g, 1.74 mmol) and N-iodosuccinimide (600.0 mg, 2.6 mmol) in acetic acid (10 ml) was stirred at 25° C. for 30 minutes. After completion, the solvent was concentrated under vacuum. The residue was purified by flash chromatography on silica gel eluting with ethyl acetate/petroleum ether (1:10) to afford 3-bromo-2,6-difluoro-4-iodoaniline (1.40 g, 2.49 mmol, 90% yield) as a purple solid. LC-MS: (ESI, m/z): 333.8 [M+H]⁺.

### Step 2: 3-bromo-2,6-difluoro-4-iodo-N,N-bis(4-methoxybenzyl)aniline

To a solution of 3-bromo-2,6-difluoro-4-iodoaniline (850.0 mg, 2.55 mmol) in N,N-dimethylformamide (10 ml) was added sodium hydride (244.4 mg, 10.18 mmol, 60% dispersion in mineral oil), the mixture was stirred at 0° C. for 15 minutes. Then 4-methoxybenzylchloride (1.40 g, 8.91 mmol) was added and stirred at 25° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (7:10) to afford 3-bromo-2,6-difluoro-4-iodo-N,N-bis(4-methoxybenzyl)aniline (1.33 g, 1.99 mmol, 78.3% yield) as a white solid. LC-MS: (ESI, m/z): 574.0[M+H]⁺.

### Step 3: 3-bromo-2,6-difluoro-N,N-bis(4-methoxybenzyl)-4-(trifluoromethyl)aniline

Under nitrogen, a solution of 3-bromo-2,6-difluoro-4-iodo-N,N-bis(4-methoxybenzyl)aniline (995.0 mg, 1.7 mmol), methyl 2,2-difluoro-2-(fluorosulfonyl)acetate (1.66 g, 8.66 mmol) and cuprous iodide (3.30 g, 17.33 mmol) in N,N-dimethylformamide (5 mL) was stirred at 90° C. for 30 minutes. After completion, the reaction was dilute with ethyl acetate. The organic layers were washed with brine, dried over anhydrous sodium sulfate and concentrated. The residue was purified by flash chromatography on silica gel eluting with petroleum ether/dichloromethane (1:5) to afford 3-bromo-2,6-difluoro-N,N-bis(4-methoxybenzyl)-4-(trifluoromethyl)aniline (920.0 mg, 1.6 mmol, 95.6% yield) as a white solid. LC-MS: (ESI, m/z): 516.3 [M+H]⁺.

### Step 4: 3-((1R)-1-(5-(3-(bis(4-methoxybenzyl)amino)-2,4-difluoro-6-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine

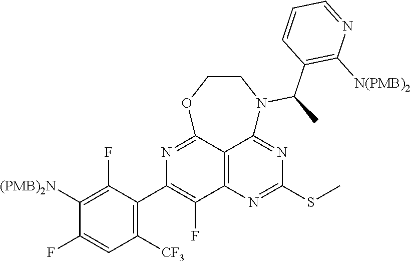

Under nitrogen, to a solution of 3-bromo-2,6-difluoro-N,N-bis(4-methoxybenzyl)-4-(trifluoromethyl)aniline (500.0 mg, 0.97 mmol) in tetrahydrofuran (5.0 ml) was added isopropyl magnesium chloride lithium chloride (1.1 ml, 1.4 mmol, 1.3 M in tetrahydrofuran) at −78° C., the mixture was stirred at −78° C. for 30 minutes. Then zinc chloride (1.6 ml, 2.91 mmol, 2 M in 2-methyltetrahydrofuran) was added at −78° C. and stirred at 25° C. for 30 minutes. The resulting mixture was added into a solution of (R)-3-(1-(5-chloro-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (626.7 mg, 0.97 mmol) and tetrakis(triphenylphosphine)palladium (111.9 mg, 0.10 mmol) in tetrahydrofuran (8 mL) and stirred at 80° C. for 3 hours under nitrogen. After completion, the reaction was diluted with water and extracted with ethyl acetate. The organic layers were combined, dried over anhydrous sodium sulfate and concentrated under vacuum. The residue was purified by flash chromatography on silica gel eluting with methanol/dichloromethane (1:8) to afford 3-((1R)-1-(5-(3-(bis(4-methoxybenzyl)amino)-2,4-difluoro-6-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (176.0 mg, 0.13 mmol, 13.9% yield) as a white solid. LC-MS: (ESI, m/z): 1048.4 [M+H]⁺.

### Step 5:_3-((1R)-1-(5-(3-(bis(4-methoxybenzyl)amino)-2,4-difluoro-6-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine

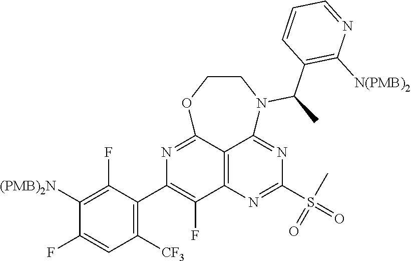

A solution of 3-((1R)-1-(5-(3-(bis(4-methoxybenzyl)amino)-2,4-difluoro-6-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (170.0 mg, 0.16 mmol) and 3-chloroperoxybenzoic acid (70.1 mg, 0.4 mmol) in dichloromethane (2 mL) was stirred at 25° C. for 1 hour. After completion, the reaction was quenched with sodium sulfite aqueous solution. The resulting solution was diluted with water and extracted with ethyl acetate. The organic layers were washed with brine, dried over anhydrous sodium sulfate and concentrated. The residue was purified by flash chromatography on silica gel eluting with ethyl acetate/petroleum ether (1:2) to afford 3-((1R)-1-(5-(3-(bis(4-methoxybenzyl)amino)-2,4-difluoro-6-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine (80.0 mg, 0.07 mmol, 41.1% yield) as a white solid. LC-MS: (ESI, m/z): 1080.3 [M+H]⁺.

### Step 6: 3-((1R)-1-(5-(3-(bis(4-methoxybenzyl)amino)-2,4-difluoro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine

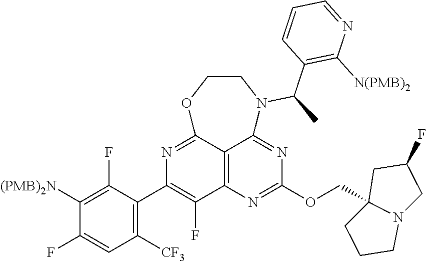

To a solution of ((2R,7aS)-2-fluorotetrahydro-1H-pyrrolizin-7a(5H)-yl)methanol (24.0 mg, 0.14 mmol) in toluene (2 mL) was added sodium tert-butoxide (21.3 mg, 0.21 mmol), the mixture was stirred at 0° C. for 10 minutes. Then 3-((1R)-1-(5-(3-(bis(4-methoxybenzyl)amino)-2,4-difluoro-6-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine (80.0 mg, 0.07 mmol) was added and stirred for 1 hour at 25° C. After completion, the solvent was concentrated under vacuum. The residue was purified by flash chromatography on silica gel eluting with ethyl acetate/petroleum ether (1:10) to afford 3-((1R)-1-(5-(3-(bis(4-methoxybenzyl)amino)-2,4-difluoro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (78.0 mg, 0.07 mmol, 90% yield) as a white solid. LC-MS: (ESI, m/z): 1159.5 [M+H]⁺.

### Step 7: 3-((1R)-1-(5-(3-amino-2,4-difluoro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

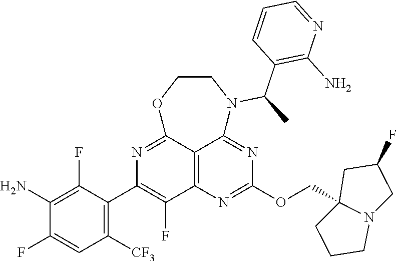 [SMILES: C[C@H](c1cccnc1N)N1CCOc2nc(-c3c(C(F)(F)F)cc(F)c(N)c3F)c(F)c3nc(OC[C@@]45CCCN4C[C@H](F)C5)nc1c23]

A solution of 3-((1R)-1-(5-(3-(bis(4-methoxybenzyl)amino)-2,4-difluoro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (78.0 mg, 0.07 mmol) in trifluoroacetic acid (1 mL) and trifluoromethanesulfonic acid (0.1 mL) was stirred at 25° C. for 5 minutes. After completion, the reaction mixture was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min; Wave Length: 254 nm/220 nm; RT1(min): 9.3 to afford 3-((1R)-1-(5-(3-amino-2,4-difluoro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (12.3 mg, 0.01 mmol, 26.9% yield) as a white solid. LC-MS: (ESI, m/z): 679.4 [M+H]⁺.

Example 621: ¹H NMR (400 MHz, DMSO-d₆, ppm) δ 7.98-7.96 (m, 1H), 7.63 (d, J=7.5 Hz, 1H), 7.48 (d, J=11.2 Hz, 1H), 6.69-6.65 (m, 1H), 6.34 (q, J=6.8 Hz, 1H), 6.23 (s, 2H), 5.68 (d, J=4.5 Hz, 2H), 5.29 (d, J=54.5 Hz, 1H), 4.50-4.38 (m, 1H), 4.37-4.26 (m, 1H), 4.14 (s, 2H), 3.79-3.63 (m, 1H), 3.42-3.37 (m, 1H), 3.17-2.98 (m, 3H), 2.89-2.77 (m, 1H), 2.24-1.94 (m, 3H), 1.90-1.71 (m, 3H), 1.56 (m, J=7.0, 2.8 Hz, 3H). LC-MS: (ESI, m/z): 679.4 [M+H]⁺.

### Example 622a: 3-((R)-1-((R)-5-(3-amino-2-chloro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Example 622b: 3-((R)-1-((S)-5-(3-amino-2-chloro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

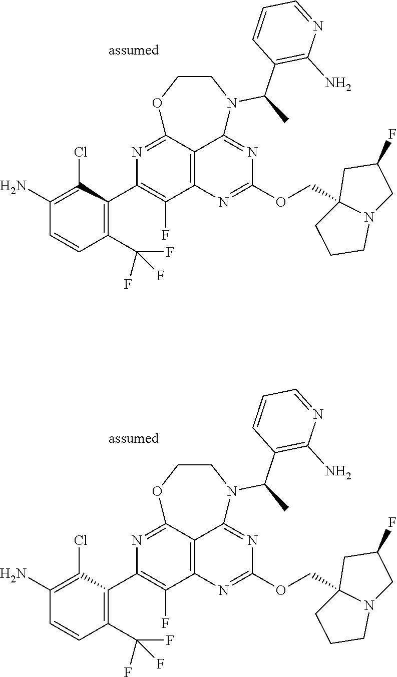

### Synthetic Route

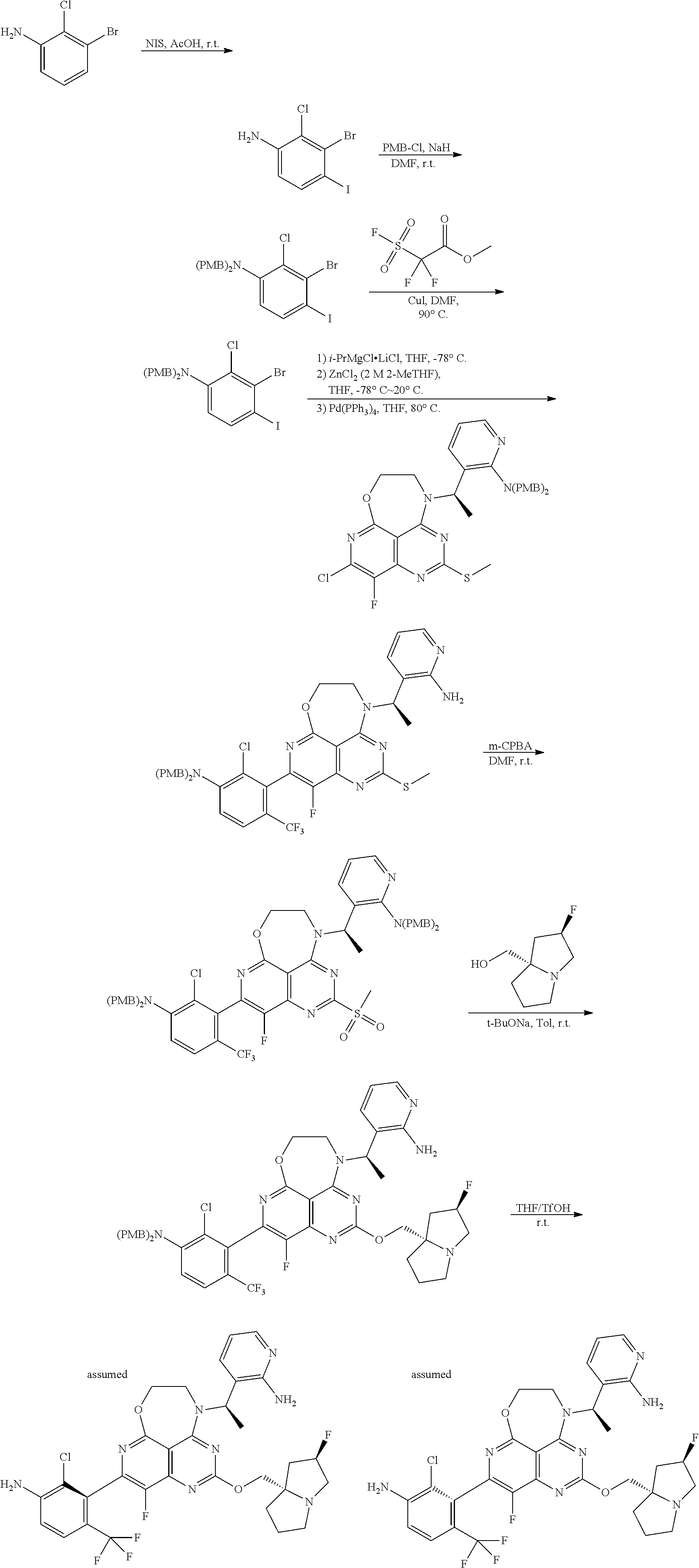

### Step 1: 3-bromo-2-chloro-4-iodoaniline

A solution of 3-bromo-2-chloro-aniline (3.00 g, 14.53 mmol) and N-iodosuccinimide (3.27 g, 14.53 mmol) in acetic acid (100 mL) was stirred at 25° C. for 1 hour. After completion, the solvent was concentrated under vacuum and diluted with dichloromethane. The reaction mixture was adjusted to pH=7 with sodium carbonate aqueous solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/petroleum ether ( 1/10) to afford 3-bromo-2-chloro-4-iodoaniline (3.80 g, 77.9% yield) as a white solid. LC-MS: (ESI, m/z):331.8 [M+H]⁺

### Step 2:_3-bromo-2-chloro-4-iodo-N,N-bis(4-methoxybenzyl)aniline

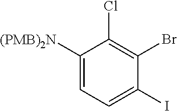

To a solution of 3-bromo-2-chloro-4-iodoaniline (3.52 g, 10.50 mmol) in N,N-dimethylacetamide (50 mL) was added sodium hydride (848.1 mg, 21.0 mmol, 60% dispersion in mineral oil), the mixture was stirred at 0° C. for 5 minutes. Then 4-methoxybenzylchloride (6.61 g, 42.00 mmol) was added and stirred at 25° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with petroleum ether/dichloromethane ( 1/10) to afford 3-bromo-2-chloro-4-iodo-N,N-bis(4-methoxybenzyl)aniline (6.00 g, 7.33 mmol) as a white oil. LC-MS: (ESI, m/z): 571.9 [M+H]⁺

### Step 3: 3-bromo-2-chloro-N,N-bis(4-methoxybenzyl)-4-(trifluoromethyl)aniline

A solution of 3-bromo-2-chloro-4-iodo-N,N-bis(4-methoxybenzyl)aniline (2.00 g, 3.49 mmol), methyl 2,2-difluoro-2-(fluorosulfonyl)acetate (2.22 mL, 17.46 mmol) and cuprous iodide (6.64 g, 34.92 mmol) in N,N-dimethylformamide (20 mL) was stirred at 90° C. for 30 minutes under nitrogen. After completion, the resulting solution was diluted with water and extracted with ethyl acetate. The organic layers were washed with brine, dried over anhydrous sodium sulfate and concentrated. The residue was purified by flash chromatography on silica gel eluting with dichloromethane/petroleum ether (⅕) to afford 3-bromo-2-chloro-N,N-bis(4-methoxybenzyl)-4-(trifluoromethyl)aniline (1.87 g) as a yellow oil. LC-MS: (ESI, m/z): 514.0 [M+H]⁺

### Step 4: 3-((1R)-1-(5-(3-(bis(4-methoxybenzyl)amino)-2-chloro-6-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine

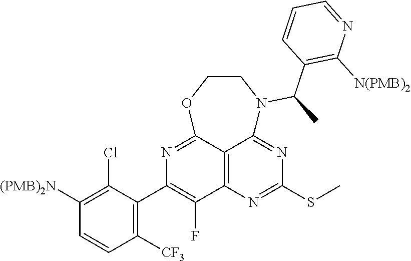

Under nitrogen, to a solution of 3-bromo-2-chloro-N,N-bis(4-methoxybenzyl)-4-(trifluoromethyl)aniline (500.0 mg, 0.97 mmol) in tetrahydrofuran (5.0 mL) was added isopropyl magnesium chloride lithium chloride (1.12 mL, 1.45 mmol, 1.3 M in tetrahydrofuran), the mixture was stirred at −78° C. for 30 minutes. Then zinc chloride (1.46 mL, 2.92 mmol, 2 M in 2-methyltetrahydrofuran) was added at −78° C. and stirred at 25° C. for 30 minutes. The resulting mixture was added into a solution of (R)-3-(1-(5-chloro-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (628.6 mg, 0.97 mmol) and tetrakis(triphenylphosphine)palladium (112.2 mg, 0.09 mmol) in tetrahydrofuran (6 mL) and stirred at 80° C. for 1 hour under nitrogen. After completion, the resulting solution was diluted with water and extracted with ethyl acetate. The organic layers were washed with brine, dried over anhydrous sodium sulfate and concentrated. The residue was purified by flash chromatography on C18 gel eluting with acetonitrile/water (6/1) to afford 3-((1R)-1-(5-(3-(bis(4-methoxybenzyl)amino)-2-chloro-6-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (190.0 mg, 0.16 mmol, 16.8% yield) as a yellow solid. LC-MS: (ESI, m/z): 1046.3 [M+H]⁺

### Step 5: 3-((1R)-1-(5-(3-(bis(4-methoxybenzyl)amino)-2-chloro-6-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine

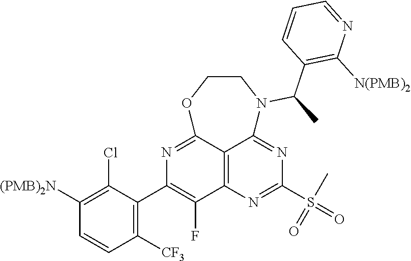

A solution of 3-((1R)-1-(5-(3-(bis(4-methoxybenzyl)amino)-2-chloro-6-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (140.0 mg, 0.13 mmol) and 3-chloroperoxybenzoic acid (57.8 mg, 0.33 mmol) in dichloromethane (2 mL) was stirred at 25° C. for 30 minutes. After completion, the reaction was quenched with sodium sulfite aqueous solution. The resulting solution was extracted with ethyl acetate and washed with brine. The organic layer was dried over anhydrous sodium sulfate and concentrated under vacuum. The residue was purified by flash chromatography on silica gel eluting with petroleum ether/ethyl acetate (⅕) to afford 3-((1R)-1-(5-(3-(bis(4-methoxybenzyl)amino)-2-chloro-6-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine (60.0 mg, 0.05 mmol, 37.4% yield) as a white solid. LC-MS: (ESI, m/z): 1078.3 [M+H]⁺

### Step 6: 3-((1R)-1-(5-(3-(bis(4-methoxybenzyl)amino)-2-chloro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine

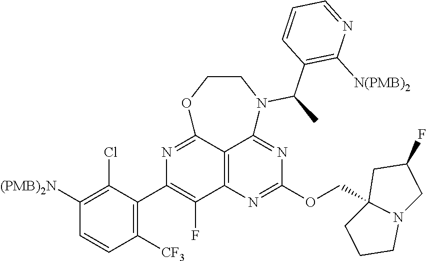

To a solution of ((2R,7aS)-2-fluorotetrahydro-1H-pyrrolizin-7a(5H)-yl)methanol (22.1 mg, 0.14 mmol) in toluene (1 mL) was added sodium tert-butoxide (13.3 mg, 0.14 mmol), the mixture was stirred at 0° C. for 15 minutes. Then 3-((1R)-1-(5-(3-(bis(4-methoxybenzyl)amino)-2-chloro-6-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine (50.0 mg, 0.05 mmol) was added and stirred at 25° C. for 1 hour. After completion, the resulting solution was diluted with water and extracted with ethyl acetate. The organic layers were washed with brine, dried over anhydrous sodium sulfate and concentrated. The residue was purified by flash chromatography on silica gel eluting with dichloromethane/ethyl acetate(1/1) to afford 3-((1R)-1-(5-(3-(bis(4-methoxybenzyl)amino)-2-chloro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (32 mg, 0.02 mmol, 50.7% yield) as a white solid. LC-MS: (ESI, m/z): 1057.4 [M+H]⁺

### Step 7: 3-((R)-1-((R)-5-(3-amino-2-chloro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine & 3-((R)-1-((S)-5-(3-amino-2-chloro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

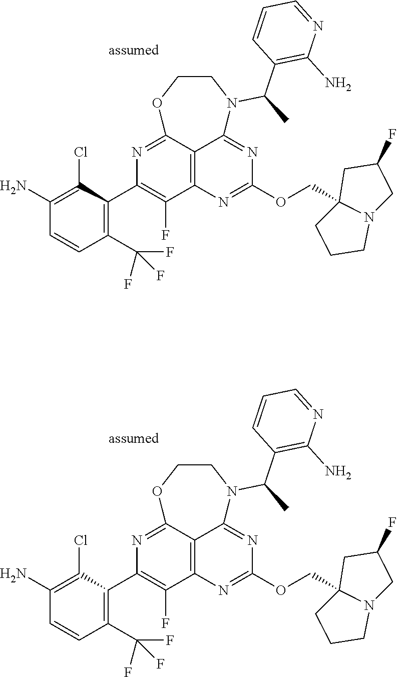

A solution of 3-((1R)-1-(5-(3-(bis(4-methoxybenzyl)amino)-2-chloro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (30.0 mg, 0.03 mmol) in trifluoromethanesulfonic acid (0.1 mL) and 2,2,2-trifluoroacetic acid (1 mL) was stirred at 25° C. for 5 minutes. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 42% B in 9 min; Wave Length: 254 nm/220 nm; RT1(min): 8.5 to afford 3-((R)-1-((R)-5-(3-amino-2-chloro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (2.4 mg, 0.003 mmol, 13.4% yield) and 3-((R)-1-((S)-5-(3-amino-2-chloro-6-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (3.4 mg, 0.004 mmol, 19% yield) as a white solid.. LC-MS: (ESI, m/z): 677.2 [M+H]⁺

Example 622a: ¹H NMR (300 MHz, Methanol-d₄, ppm) δ 7.98 (dd, J=5.0, 1.7 Hz, 1H), 7.78 (d, J=7.5 Hz, 1H), 7.49 (d, J=8.8 Hz, 1H), 7.01 (d, J=8.7 Hz, 1H), 6.80 (dd, J=7.6, 5.1Hz, 1H), 6.63 (q, J=6.9 Hz, 1H), 5.33 (d, J=53.8 Hz, 1H), 4.49 (dd, J=12.7, 6.3 Hz, 1H), 4.37 (dd, J=12.7, 6.3 Hz, 1H), 4.34 (s, 2H), 3.79 (dd, J=16.0, 6.4 Hz, 1H), 3.56 (dd, J=16.0, 6.4 Hz, 1H), 3.31-3.16 (m, 2H), 3.12-2.98 (m, 1H), 2.41 (dd, J=15.0, 4.6 Hz, 1H), 2.33-2.09 (m, 3H), 2.09-1.87 (m, 3H), 1.67 (d, J=6.9 Hz, 3H). LC-MS: (ESI, m/z): 677.2 [M+H]⁺

Example 622b: ¹H NMR (300 MHz, Methanol-d₄, ppm) δ 7.98 (dd, J=5.2, 1.6 Hz, 1H), 7.78 (d, J=7.5 Hz, 1H), 7.49 (d, J=8.8 Hz, 1H), 7.01 (d, J=8.6 Hz, 1H), 6.80 (dd, J=7.5, 5.1Hz, 1H), 6.63 (q, J=6.9 Hz, 1H), 5.33 (d, J=53.7 Hz, 1H), 4.49 (dd, J=12.8, 6.2 Hz, 1H), 4.36 (d, J=11.0 Hz, 3H), 3.78 (dd, J=16.1, 6.5 Hz, 1H), 3.58 (dd, J=16.0, 6.2 Hz, 1H), 3.30-3.19 (m, 2H), 3.11-2.96 (m, 1H), 2.41 (dd, J=15.0, 4.5 Hz, 1H), 2.34-2.08 (m, 3H), 2.11-1.80 (m, 3H), 1.67 (d, J=6.9 Hz, 3H). LC-MS: (ESI, m/z):677.2 [M+H]⁺

### Example 623: 3-((R)-1-(5-(5-amino-3,4-difluoro-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Synthetic Route

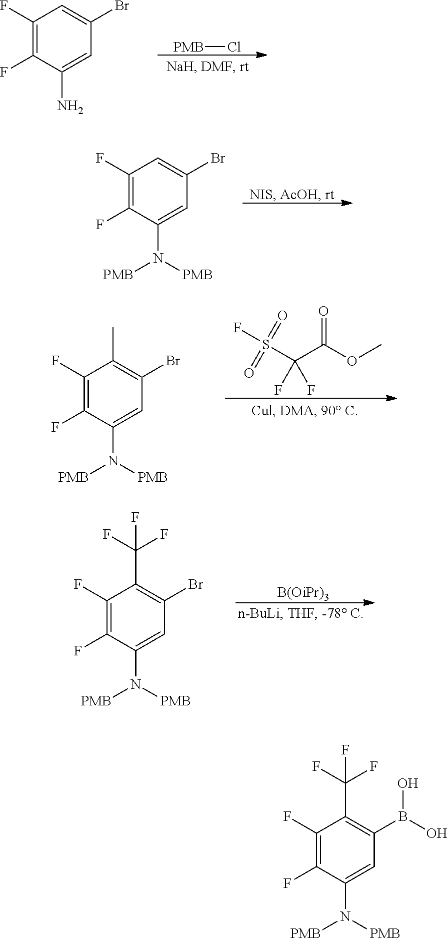

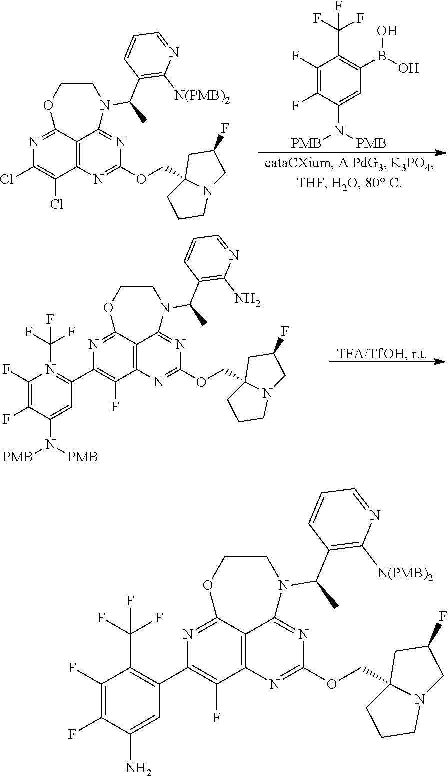

### Step 1: 5-bromo-2,3-difluoro-N,N-bis(4-methoxybenzyl)aniline

To a solution of 5-bromo-2,3-difluoroaniline (1.00 g, 4.80 mmol) in N,N-dimethylformamide (10 mL) was added sodium hydride (769.2 mg, 19.20 mmol, 60% dispersion in mineral oil) and stirred at 0° C. for 30 minutes. Then 4-methoxybenzylchloride (1.60 g, 10.50 mmol) was added and stirred at 25° C. for 1 hour. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1/1) to afford 5-bromo-2,3-difluoro-N,N-bis(4-methoxybenzyl)aniline (1.90 g, 4.24 mmol, 70% yield) as a yellow oil. LC-MS: (ESI, m/z): 448.1 [M+H]⁺

### Step 2: 5-bromo-2,3-difluoro-4-iodo-N,N-bis(4-methoxybenzyl)aniline

A solution of 5-bromo-2,3-difluoro-N,N-bis(4-methoxybenzyl)aniline (1.90 g, 4.24 mmol) and N-iodosuccinimide (1.05 g, 4.67 mmol) in acetic acid (25 mL) was stirred at 25° C. for 1 hour. After completion, the reaction was quenched with saturated sodium thiosulfate solution. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1/1) to afford 5-bromo-2,3-difluoro-4-iodo-N,N-bis(4-methoxybenzyl)aniline (2.25 g, 3.91 mmol, 92% yield) as a yellow solid. LC-MS: (ESI, m/z): 574.0 [M+H]⁺

### Step 3: 5-bromo-2,3-difluoro-N,N-bis(4-methoxybenzyl)-4-(trifluoromethyl)aniline

Under nitrogen, a solution of 5-bromo-2,3-difluoro-4-iodo-N,N-bis(4-methoxybenzyl)aniline (2.25 g, 3.91 mmol), methyl 2,2-difluoro-2-(fluoro-2-(fluorosulfonyl)acetate (7.51 g, 39.10 mmol) and copper(I) iodide (7.44 g, 39.10 mmol) in N,N-dimethylformamide (30.0 mL) was stirred at 90° c. for 2 hours. After completion, the solids were filtered out. The filtrate was diluted with water and extracted with ethyl acetate. The organic layers were dried over anhydrous sodium sulfate and concentrated under vacuum. The residue was purified by flash chromatography on silica gel eluting with dichloromethane/ethyl acetate (10:1) to afford 5-bromo-2,3-difluoro-N,N-bis(4-methoxybenzyl)-4-(trifluoromethyl)aniline (1.83 g, 3.54 mmol, 90% yield). LC-MS: (ESI, m/z): 516.1 [M+H]⁺

### Step 4: (5-(bis(4-methoxybenzyl)amino)-3,4-difluoro-2-(trifluoromethyl)phenyl)boronic acid

Under nitrogen, to a solution of 5-bromo-2,3-difluoro-N,N-bis(4-methoxybenzyl)-4-(trifluoromethyl)aniline (410.0 mg, 0.70 mmol) in tetrahydrofuran (5 mL) was added n-butyllithium (0.37 ml, 0.9 mmol, 2.5 M in hexane) and stirred at −78° C. for 10 minutes. Then triisopropyl borate (447.8 mg, 2.30 mmol) was added and stirred at −78° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (10:1) to afford (5-(bis(4-methoxybenzyl)amino)-3,4-difluoro-2-(trifluoromethyl)phenyl)boronic acid (260.0 mg, 0.5 mmol, 68% yield) as a brown solid. LC-MS: (ESI, m/z): 482.1 [M+H]⁺

### Step 5: 3-((R)-1-(5-(5-(bis(4-methoxybenzyl)amino)-3,4-difluoro-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

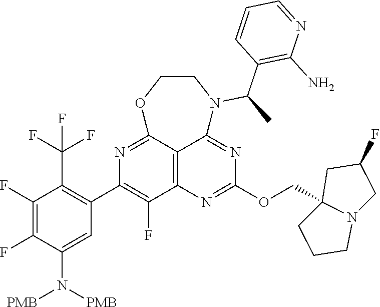

Under nitrogen atmosphere, a solution of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (228.0 mg, 0.3 mmol), (5-(bis(4-methoxybenzyl)amino)-3,4-difluoro-2-(trifluoromethyl)phenyl)boronic acid (229.1 mg, 0.4 mmol), mesylate[(di(1-adamantyl)-n-butylphosphine)-2-(2′-amino-1,1′-biphenyl)]palladium(II) (22.9 mg, 0.03 mmol) and potassium phosphate (201.9 mg, 0.9 mmol) in tetrahydrofuran (2 mL) and water (0.4 mL) was added and stirred at 80° C. for 2 hours. After completion, the reaction mixture was diluted with water, extracted with ethyl acetate and the organic layers were combined. The organic layer was dried over anhydrous sodium sulfate and concentrated under vacuum. The residue was purified by flash chromatography on silica gel eluting with dichloromethane/methanol (10:1) to afford 3-((R)-1-(5-(5-(bis(4-methoxybenzyl)amino)-3,4-difluoro-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (200.0 mg, 0.10 mmol, 56.3% yield) as a white solid. LC-MS: (ESI, m/z): 919.4 [M+H]⁺

### Step 6: 3-((R)-1-(5-(5-amino-3,4-difluoro-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

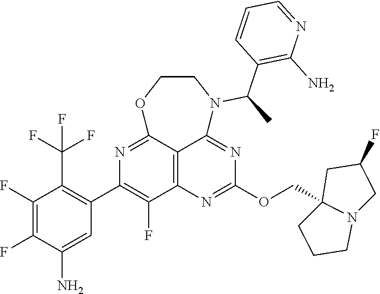 [SMILES: C[C@H](c1cccnc1N)N1CCOc2nc(-c3cc(N)c(F)c(F)c3C(F)(F)F)c(F)c3nc(OC[C@@]45CCCN4C[C@H](F)C5)nc1c23]

A solution of 3-((R)-1-(5-(5-(bis(4-methoxybenzyl)amino)-3,4-difluoro-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (180.0 mg, 0.10 mmol) in trifluoroacetic acid (1.5 mL) and trifluoromethanesulfonic acid (0.15 mL) was stirred at 25° C. for 5 minutes. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: 20 mm ACN; Flow rate: 60 mL/min; Gradient: 35% B to 65% B in 8 min; Wave Length: 254 nm/220 nm; RT1(min): 6.9 to afford 3-((R)-1-(5-(5-amino-3,4-difluoro-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (46.3 mg,0.06 mmol, 42% yield) as a white solid. LC-MS: (ESI, m/z): 679.3 [M+H]⁺

Example 623: ¹H NMR (400 MHz, DMSO-d₆, ppm) δ 7.97 (dd, J=4.9, 1.7 Hz, 1H), 7.62 (dd, J=7.6, 1.8 Hz, 1H), 6.67 (dd, J=7.5, 4.9 Hz, 1H), 6.58 (dd, J=7.9, 1.6 Hz, 1H), 6.54 (s, 2H), 6.33 (q, J=6.9 Hz, 1H), 5.70 (s, 2H), 5.29 (d, J=54.4 Hz, 1H), 4.43 (dd, J=12.7, 6.7 Hz, 1H), 4.30 (dd, J=12.7, 6.6 Hz, 1H), 4.13 (s, 2H), 3.72 (dd, J=15.9, 6.6 Hz, 1H), 3.40 (dd, J=15.9, 6.6 Hz, 1H), 3.13-2.98 (m, 3H), 2.86-2.80 (m, 1H), 2.17-2.00 (m, 3H), 1.87-1.76 (m, 3H), 1.56 (d, J=6.9 Hz, 3H). LCMS (ESI, m/z): 679.3 [M+H]⁺.

### Example 624: 3-((1R)-1-(5-(3-amino-2,4-difluoro-5-methyl-6-(trifluoromethyl)phenyl)-4-fluoro-2-(((S)-1-methylpyrrolidin-2-yl)methoxy)-8,9-dihydro-10H-7-oxa-1,3,6,10-tetraazacyclohepta[de]naphthalen-10-yl)ethyl)pyridin-2-amine

### Synthetic Route

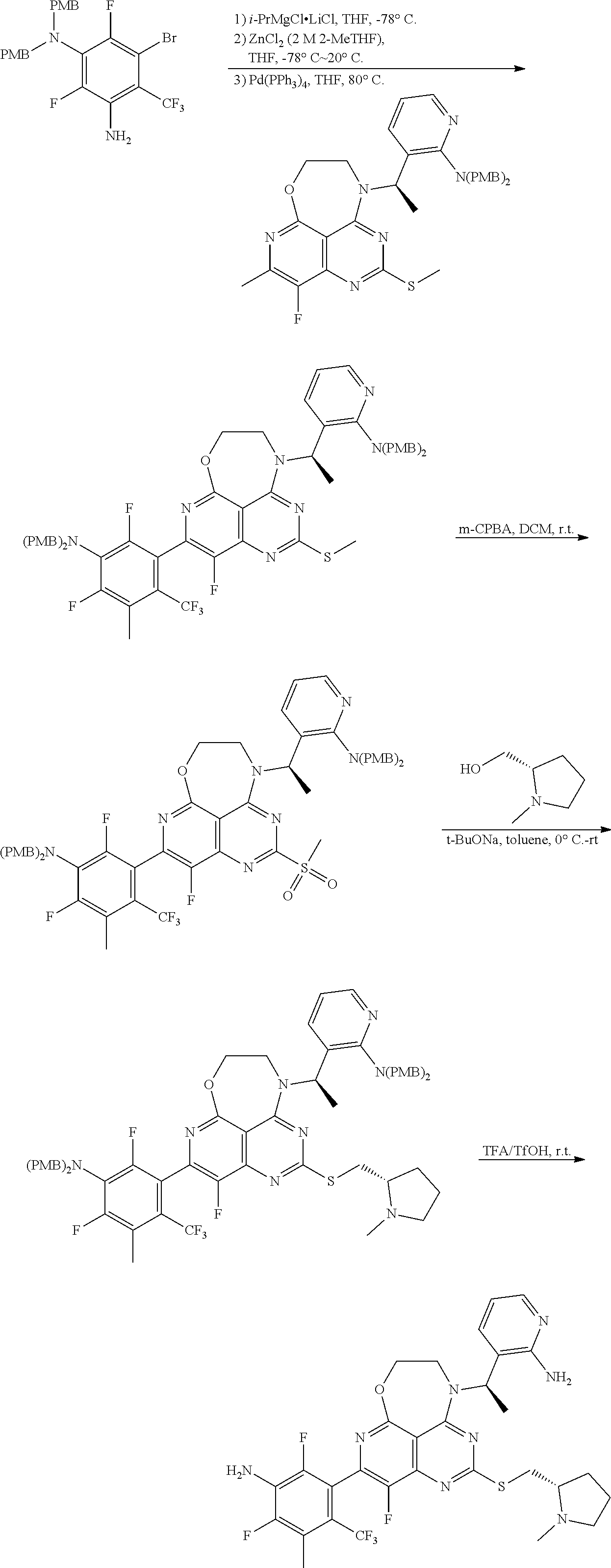

### Step 1: 3-((1R)-1-(5-(3-(bis(4-methoxybenzyl)amino)-2,4-difluoro-5-methyl-6-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine

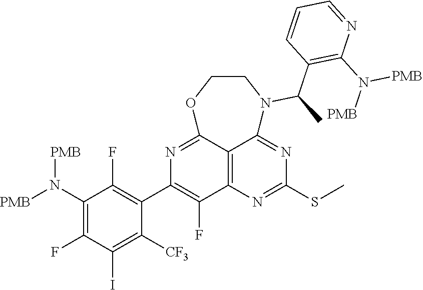

Under nitrogen, to a solution of 3-bromo-2,6-difluoro-N,N-bis(4-methoxybenzyl)-5-methyl-4-(trifluoromethyl)aniline (1.20 g, 2.3 mmol) in tetrahydrofuran (13 mL) was added isopropyl magnesium chloride lithium chloride (2.6 mL, 3.4 mmol, 1.3 M in tetrahydrofuran), the mixture was stirred at −78° C. for 30 minutes. Then zinc chloride (3.4 mL, 6.8 mmol, 2 M in 2-methyltetrahydrofuran) was added at −78° C. and stirred at 25° C. for 30 minutes. The resulting mixture was added into a solution of (R)-3-(1-(5-chloro-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (1.40 g, 2.3 mmol) and tetrakis(triphenylphosphine)palladium (0.30 g, 0.2 mmol) in tetrahydrofuran (13 mL) and stirred at 80° C. for 5 hours. After completion, the reaction was quenched with saturated ammonium chloride solution, diluted with water, extracted with ethyl acetate and the organic layers were combined. The organic layer was dried over anhydrous sodium sulfate and concentrated under vacuum. The residue was purified by flash chromatography on silica gel eluting with methanol/dichloromethane (1:8) to afford 3-((1R)-1-(5-(3-(bis(4-methoxybenzyl)amino)-2,4-difluoro-5-methyl-6-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (600.0 mg, 0.5 mmol, 25% yield) as a white solid. LC-MS: (ESI, m/z): 1062.4 [M+H]⁺

### Step 2: 3-((1R)-1-(5-(3-(bis(4-methoxybenzyl)amino)-2,4-difluoro-5-methyl-6-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine

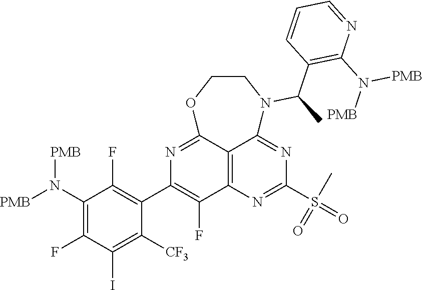

Under nitrogen, a solution of 3-((1R)-1-(5-(3-(bis(4-methoxybenzyl)amino)-2,4-difluoro-5-methyl-6-(trifluoromethyl)phenyl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (100.0 mg, 0.1 mmol) and 3-chloroperoxybenzoic acid (48.7 mg, 0.3 mmol) in dichloromethane (1 mL) was stirred at 25° C. for 1.5 hours. After completion, the reaction was quenched with saturated sodium sulfite solution. The resulting solution was extracted with ethyl acetate, washed with water dried over anhydrous sodium sulfate and concentrated under vacuum. The residue was purified by flash chromatography on silica gel eluting with methanol/dichloromethane (1:8) to afford 3-((1R)-1-(5-(3-(bis(4-methoxybenzyl)amino)-2,4-difluoro-5-methyl-6-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine (70.0 mg, 0.06 mmol, 68% yield) as a white solid. LC-MS: (ESI, m/z): 1094.4 [M+H]⁺

### Step 3: 3-((1R)-1-(5-(3-(bis(4-methoxybenzyl)amino)-2,4-difluoro-5-methyl-6-(trifluoromethyl)phenyl)-4-fluoro-2-(((S)-1-methylpyrrolidin-2-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine

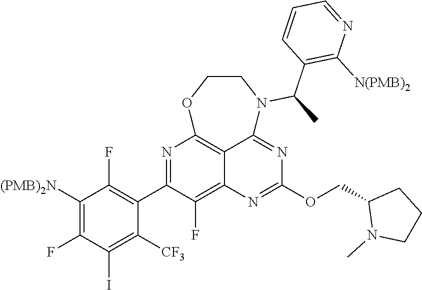

To a solution of (S)-(1-methylpyrrolidin-2-yl)methanol (50.4 mg, 0.44 mmol) in toluene (1 ml) was added sodium tert-butoxide (63.0 mg, 0.66 mmol), the mixture was stirred at 25° C. for 10 minutes. Then 3-((1R)-1-(5-(3-(bis(4-methoxybenzyl)amino)-2,4-difluoro-5-methyl-6-(trifluoromethyl)phenyl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine (240.0 mg, 0.22 mmol) was added and stirred at 25° C. for 30 minutes. The resulting solution was diluted with water and extracted with ethyl acetate. The organic layers were washed with brine, dried over anhydrous sodium sulfate and concentrated. The residue was purified by flash chromatography on silica gel eluting with dichloromethane/ethyl acetate (1:10) to afford 3-((1R)-1-(5-(3-(bis(4-methoxybenzyl)amino)-2,4-difluoro-5-methyl-6-(trifluoromethyl)phenyl)-4-fluoro-2-(((S)-1-methylpyrrolidin-2-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (130.0 mg, 0.1 mmol, 47.2% yield) as a white oil. LC-MS: (ESI, m/z): 1129.2 [M+H]⁺.

### Step 4: 3-((1R)-1-(5-(3-amino-2,4-difluoro-5-methyl-6-(trifluoromethyl)phenyl)-4-fluoro-2-(((S)-1-methylpyrrolidin-2-yl)methoxy)-8,9-dihydro-10H-7-oxa-1,3,6,10-tetraazacyclohepta[de]naphthalen-10-yl)ethyl)pyridin-2-amine

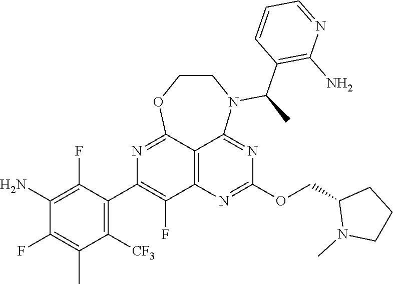 [SMILES: Cc1c(F)c(N)c(F)c(-c2nc3c4c(nc(OC[C@@H]5CCCN5C)nc4c2F)N([C@H](C)c2cccnc2N)CCO3)c1C(F)(F)F]

A solution of 3-((1R)-1-(5-(3-(bis(4-methoxybenzyl)amino)-2,4-difluoro-5-methyl-6-(trifluoromethyl)phenyl)-4-fluoro-2-(((S)-1-methylpyrrolidin-2-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (120.1 mg, 0.1 mmol) in trifluoroacetic acid (1 mL) and trifluoromethanesulfonic (0.1 mL) was stirred at 25° C. for 5 minutes. After completion, the solvent was concentrated under vacuum. The reaction mixture was adjusted to pH=7 with sodium carbonate aqueous solution. The resulting solution was extracted with ethyl acetate, dried over anhydrous sodium sulfate and concentrated. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 31% B to 53% B in 10 min; Wave Length: 254 nm/220 nm; RT1(min): 10 to afford 3-((1R)-1-(5-(3-amino-2,4-difluoro-5-methyl-6-(trifluoromethyl)phenyl)-4-fluoro-2-(((S)-1-methylpyrrolidin-2-yl)methoxy)-8,9-dihydro-10H-7-oxa-1,3,6,10-tetraazacyclohepta[de]naphthalen-10-yl)ethyl)pyridin-2-amine (21.9 mg, 0.03 mmol, 31.6% yield) as a white solid. LC-MS: (ESI, m/z): 649.3 [M+H]⁺.

Example 624: ¹H NMR (300 MHz, Methanol-d₄, ppm) δ 7.96 (dd, J=5.1, 1.6 Hz, 1H), 7.76 (d, J=7.5 Hz, 1H), 6.78 (dd, J=7.5, 5.1Hz, 1H), 6.61 (q, J=6.8 Hz, 1H), 4.52 (d, J=5.6 Hz, 2H), 4.49-4.28 (m, 2H), 3.74 (dd, J=16.0, 6.4 Hz, 1H), 3.57-3.47 (m, 1H), 3.15-3.04 (m, 1H), 2.86-2.75 (m, 1H), 2.52 (s, 3H), 2.40-2.32 (m, 4H), 2.13-2.00 (m, 1H), 1.94-1.70 (m, 3H), 1.65 (d, J=6.9 Hz, 3H). LC-MS: (ESI, m/z): 649.3 [M+H]⁺

### Example 626: 4-(10-((R)-1-(2-aminopyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-6-methyl-5-(trifluoromethyl)pyridin-2-amine

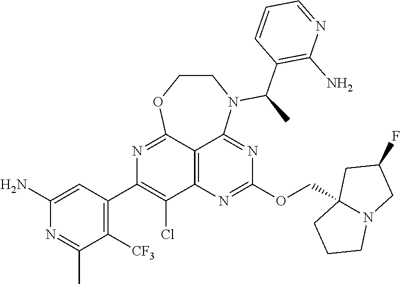 [SMILES: Cc1nc(N)cc(-c2nc3c4c(nc(OC[C@@]56CCCN5C[C@H](F)C6)nc4c2Cl)N([C@H](C)c2cccnc2N)CCO3)c1C(F)(F)F]

### Synthetic Route

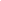

### Step 1: 2,3,6-trichloropyridin-4-amine

A solution of 2,6-dichloropyridin-4-amine (25.00 g, 153.37 mmol) and N-chlorosuccinimide (20.48 g, 153.33 mmol) in tetrahydrofuran (1000 mL) was stirred at 50° C. for 26 hours. After completion, the solvent was concentrated under vacuum. The resulting solution was diluted with water and extracted with ethyl acetate. The organic layers were combined and concentrated. Then the crude product was stirred in petroleum ether (200 mL) and ethyl acetate (20 mL) at room temperature for 0.5 h. After filtration, the solids were collected and washed by petroleum ether to afford 2,3,6-trichloropyridin-4-amine (26.70 g, 135.22 mmol, 88.2% yield) as a white solid. LC-MS: (ESI, m/z): 197.0 [M+H]⁺

### Step 2: 2,3,6-trichloro-5-iodopyridin-4-amine

A solution of 2,3,6-trichloropyridin-4-amine (25.40 g, 128.64 mmol), N-iodosuccinimide (34.72 g, 154.33 mmol) and p-toluenesulfonic acid (2.22 g, 12.86 mmol) in acetonitrile (300 mL) was stirred 2 hours at 70° C. After completion, the solvent was concentrated under vacuum. The resulting solution diluted with ethyl acetate and washed with brine. The organic layers were combined and concentrated. The residue was purified by flash chromatography on silica gel eluting with petroleum ether/ethyl acetate (3:1) to afford 2,3,6-trichloro-5-iodo-pyridin-4-amine (38.1 g, 117.83 mmol, 91.6% yield) as a white solid. LC-MS: (ESI, m/z): 322.8 [M+H]⁺

### Step 3: ethyl 4-amino-2,5,6-trichloronicotinate

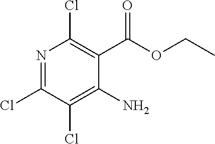

A mixture of 2,3,6-trichloro-5-iodo-pyridin-4-amine (35.30 g, 109.17 mmol), bis(triphenylphosphine)palladium(II) chloride (7.66 g, 10.91 mmol) and triethylamine (33.11 g, 327.20 mmol) in ethanol (350 mL) was stirred for 30 hours at 80° C. under carbon monoxide atmosphere. After completion, the resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (4:1) to afford ethyl 4-amino-2,5,6-trichloronicotinate (21.30 g, 79.03 mmol, 72.4% yield) as an orange solid. LC-MS: (ESI, m/z): 269.1 [M+H]⁺

### Step 4: 4-amino-2,5,6-trichloronicotinic acid

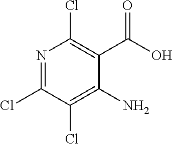 [SMILES: Nc1c(Cl)c(Cl)nc(Cl)c1C(=O)O]

A solution of ethyl 4-amino-2,5,6-trichloronicotinate (19.16 g, 71.08 mmol) and lithium hydroxide (3.41 g, 142.48 mmol) in methyl alcohol (120 mL), water (30 mL) and tetrahydrofuran (60 mL) was stirred at 25° C. for 1 hour. After completion, the resulting mixture was filtered. The filtrate was acidified to pH=3 with hydrochloric acid (6 mol/L). The resulting solution was diluted with water and extracted with ethyl acetate. The organic layers were washed with brine, dried over anhydrous sodium sulfate and concentrated to afford 4-amino-2,5,6-trichloro-pyridine-3-carboxylic acid (17 g, 70.40 mmol, 99% yield) as a white solid. LC-MS: (ESI, m/z): 240.9 [M+H]⁺

### Step 5: 5,7,8-trichloro-2-thioxo-2,3-dihydropyrido[4,3-d]pyrimidin-4(1H)-one

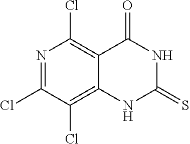 [SMILES: O=c1[nH]c(=S)[nH]c2c(Cl)c(Cl)nc(Cl)c12]

A solution of 4-amino-2,5,6-trichloro-pyridine-3-carboxylic acid (10.00 g, 41.41 mmol) in acetonitrile (90 mL) was added cyanic (ethyl carbonic) thioanhydride (21.80 g, 166.22 mmol) in pyridine (30 mL) at 0° C. The resulting solution was stirred at room temperature for 4 hours. After filtration, the solids were collected and washed with acetonitrile. The solid was diluted with methanol. The solvent was concentrated under vacuum to afford 5,7,8-trichloro-2-thioxo-2,3-dihydropyrido[4,3-d]pyrimidin-4(1H)-one (7.41 g, 26.23 mmol, 63.3% yield) as a yellow solid. LC-MS: (ESI, m/z): 279.8 [M−H]⁻

### Step 6: 5,7,8-trichloro-2-(methylthio)pyrido[4,3-d]pyrimidin-4(3H)-one

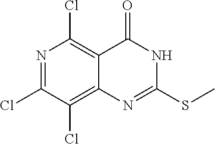 [SMILES: CSc1nc2c(Cl)c(Cl)nc(Cl)c2c(=O)[nH]1]

A solution of 5,7,8-trichloro-2-thioxo-2,3-dihydropyrido[4,3-d]pyrimidin-4(1H)-one (6.46 g, 22.87 mmol), sodium methoxide (1.11 g, 20.57 mmol) in N,N-dimethylformamide (65 mL) was stirred at room temperature for 1 hour. Then iodomethane (3.90 g, 27.45 mmol) was added and stirred at room temperature for 2 hours. After completion, the reaction mixture was diluted with water and extracted with dichloromethane. The organic layers were combined, dried over anhydrous sodium sulfate and concentrated under vacuum to afford 5,7,8-trichloro-2-(methylthio)pyrido[4,3-d]pyrimidin-4(3H)-one (5.01 g, 16.89 mmol, 73.9% yield) as a yellow solid. LC-MS: (ESI, m/z): 295.9 [M+H]⁺

### Step 7: (R)-5-(2-((1-(2-(bis(4-methoxybenzyl)amino)pyridin-3-yl)ethyl)amino)ethoxy)-7,8-dichloro-2-(methylthio)pyrido[4,3-d]pyrimidin-4(3H)-one

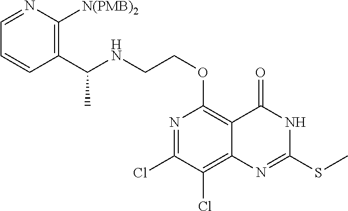

To a solution of (R)-2-((1-(2-(bis(4-methoxybenzyl)amino)pyridin-3-yl)ethyl)amino)ethan-1-ol (1.09 g, 2.58 mmol) in tetrahydrofuran (8 mL) was added sodium hydride (380.0 mg, 9.5 mmol, 60% dispersion in mineral oil) at 0° C. The resulting solution was stirred for 10 minutes at 25° C. Then 5,7,8-trichloro-2-(methylthio)pyrido[4,3-d]pyrimidin-4(3H)-one (700.0 mg, 2.36 mmol) was added and stirred at 25° C. for 1 hour. After completion, the resulting solution was diluted with ethyl acetate and washed with water. The organic layer was washed with brine and dried over anhydrous sodium sulfate and concentrated under vacuum. The residue was purified by flash chromatography on silica gel eluting with dichloromethane/methanol (9:1) to afford (R)-5-(2-((1-(2-(bis(4-methoxybenzyl)amino)pyridin-3-yl)ethyl)amino)ethoxy)-7,8-dichloro-2-(methylthio)pyrido[4,3-d]pyrimidin-4(3H)-one (1.52 g, 2.24 mmol, 94.7% yield) as an orange solid. LC-MS: (ESI, m/z): 681.1 [M+H]⁺

### Step 8: (R)-3-(1-(4,5-dichloro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine

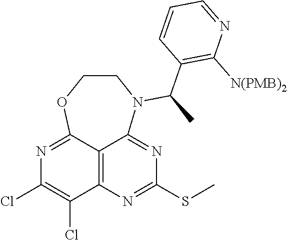

A solution of (R)-5-(2-((1-(2-(bis(4-methoxybenzyl)amino)pyridin-3-yl)ethyl)amino)ethoxy)-7,8-dichloro-2-(methylthio)pyrido[4,3-d]pyrimidin-4(3H)-one (1.50 g, 2.20 mmol), bis(2-oxo-3-oxazolidinyl)phosphinic chloride (1.68 g, 6.60 mmol) and N,N-diisopropylethylamine (855.0 mg, 6.62 mmol) in chloroform (15 mL) was stirred at 70° C. for 1 hour. After completion, the resulting solution was diluted with dichloromethane and washed with water. The organic layer was washed with brine and dried over anhydrous sodium sulfate and concentrated under vacuum. The residue was purified by flash chromatography on silica gel eluting with dichloromethane/methanol (9:1) to afford (R)-3-(1-(4,5-dichloro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (900.0 mg, 1.35 mmol, 61.6% yield) as a yellow solid. LC-MS: (ESI, m/z): 663.1 [M+H]⁺

### Step 9: (R)-3-(1-(4,5-dichloro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine

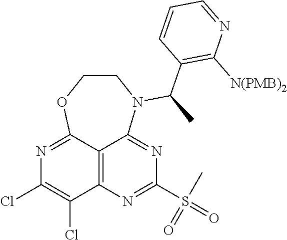

A solution of (R)-3-(1-(4,5-dichloro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (780.0 mg, 1.18 mmol) and 3-chloroperoxybenzoic acid (508.7 mg, 2.94 mmol) in dichloromethane (7.8 mL) was stirred at 25° C. for 1 hour. After completion, the reaction was quenched with saturated sodium sulfite solution. The reaction mixture was diluted with water. The resulting solution was extracted with ethyl acetate and the organic layers were combined. The organic layer was dried over anhydrous sodium sulfate and concentrated under vacuum. The residue was purified by flash chromatography on silica gel eluting with dichloromethane/methanol (10:1) to afford (R)-3-(1-(4,5-dichloro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine (500.0 mg, 0.63 mmol, 54.3% yield) as a white solid. LC-MS: (ESI, m/z): 695.6 [M+H]⁺

### Step 10: 3-((R)-1-(4,5-dichloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine

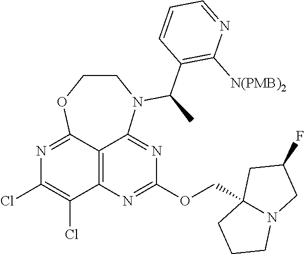

A solution of ((2R,7aS)-2-fluorotetrahydro-1H-pyrrolizin-7a(5H)-yl)methanol (211.6 mg, 1.33 mmol) and sodium tert-butoxide (190.4 mg, 1.19 mmol) in toluene (5 mL) was stirred at 0° C. for 20 minutes. Then (R)-3-(1-(4,5-dichloro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine (460.0 mg, 0.66 mmol) was added and stirred at 0° C. for 15 minutes. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (10:1) to afford 3-((R)-1-(4,5-dichloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (280.0 mg, 0.32 mmol, 48.6% yield) as a white solid. LC-MS: (ESI, m/z): 774.7 [M+H]⁺

### Step 11: 4-(10-((R)-1-(2-(bis(4-methoxybenzyl)amino)pyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-6-methyl-5-(trifluoromethyl)pyridin-2-amine

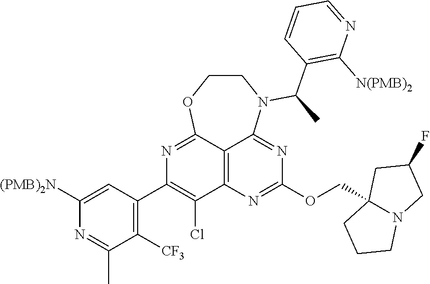

Under nitrogen, a solution of potassium phosphate (165.0 mg, 0.77 mmol), 3-((R)-1-(4,5-dichloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (200.0 mg, 0.26 mmol), N,N-bis(4-methoxybenzyl)-6-methyl-4-(4,4,5,5-tetramethyl-1,3,2-dioxaborolan-2-yl)-5-(trifluoromethyl)pyridin-2-amine (129.0 mg, 0.31 mmol) and mesylate[(di(1-adamantyl)-n-butylphosphine)-2-(2′-amino-1,1′-biphenyl)]palladium(II) (19.0 mg, 0.03 mmol) in 1,4-dioxane (2 mL) and water (0.2 mL) was stirred at 100° C. for 1 hour. After completion, the solvent was concentrated under vacuum. The reaction mixture was diluted with water. The resulting solution was extracted with ethyl acetate and the organic layers were combined. The organic layer was dried over anhydrous sodium sulfate and concentrated under vacuum. The resulting residue was purified by reverse phase chromatography (acetonitrile/0.1% ammonium bicarbonate in water) to afford 4-(10-((R)-1-(2-(bis(4-methoxybenzyl)amino)pyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-6-methyl-5-(trifluoromethyl)pyridin-2-amine (60.0 mg, 0.04 mmol, 17.3% yield) as a white solid. LC-MS: (ESI, m/z): 1154.7[M+H]⁺

### Step 12: 4-(10-((R)-1-(2-aminopyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-6-methyl-5-(trifluoromethyl)pyridin-2-amine

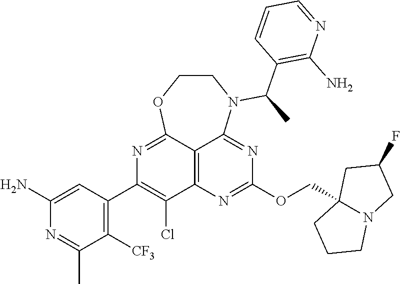 [SMILES: Cc1nc(N)cc(-c2nc3c4c(nc(OC[C@@]56CCCN5C[C@H](F)C6)nc4c2Cl)N([C@H](C)c2cccnc2N)CCO3)c1C(F)(F)F]

A solution of 4-(10-((R)-1-(2-(bis(4-methoxybenzyl)amino)pyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-6-methyl-5-(trifluoromethyl)pyridin-2-amine (55.0 mg, 0.05 mmol) in trifluoroacetic acid (0.55 mL) and trifluoromethanesulfonic acid (0.05 mL) was stirred at 25° C. for 20 minutes. After completion, the reaction mixture was concentrated under vacuum, diluted with ethyl acetate, adjusted to pH>7 with sodium bicarbonate saturated solution, extracted with ethyl acetate and the organic layer was combined. The organic layer was dried over anhydrous sodium sulfate and concentrated under vacuum. The product was purified by Prep-HPLC with the following conditions: Column: YMC Triart C18 ExRs Sum, 30*154 mm; Mobile Phase A: Water(10 mmol/L NH₄HCO₃ and 0.1% NH₃H₂O), Mobile Phase B: ACN; Flow rate: 60 mL/min; Gradient: 36% B to 59% B in 10 min; Wave Length: 254 nm/230 nm to afford 4-(10-((R)-1-(2-aminopyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-6-methyl-5-(trifluoromethyl)pyridin-2-amine (11.8 mg, 0.02 mmol, 36% yield) as a white solid. LC-MS: (ESI, m/z): 674.2[M+H]⁺

Example 626: ¹H NMR (300 MHz, DMSO-d₆, ppm) δ 7.97 (d, J=4.9 Hz, 1H), 7.62 (d, J=7.4 Hz, 1H), 6.78 (s, 2H), 6.71-6.63 (m, 1H), 6.35 (q, J=7.0 Hz, 1H), 6.08 (s, 1H), 5.69 (s, 2H), 5.29 (d, J=53.9 Hz, 1H), 4.52-4.38 (m, 1H), 4.37-4.25 (m, 1H), 4.23-4.09 (m, 2H), 3.79-3.64 (m, 1H), 3.44-3.36 (m, 1H), 3.17-3.00 (m, 3H), 2.88-2.76 (m, 1H), 2.47 (s, 3H), 2.20-2.13 (m, 1H), 2.11-1.96 (m, 2H), 1.91-1.72 (m, 3H), 1.55 (t, J=5.9 Hz, 3H). LC-MS: (ESI, m/z): 674.2[M+H]⁺

### Example 627: 4-(10-((R)-1-(2-aminopyridin-3-yl)ethyl)-4-fluoro-2-(((S)-1-methylpyrrolidin-2-yl)methoxy)-9,10-dihydro-8H-7-oxa-1,3,6,10-tetraazacyclohepta[de]naphthalen-5-yl)-6-methyl-5-(trifluoromethyl)pyridin-2-amine

### Synthetic Route

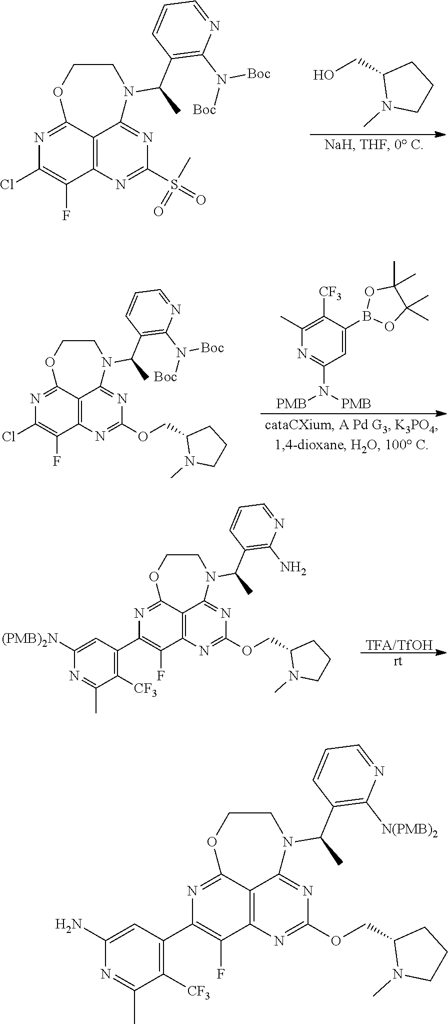

### Step 1: tert-butyl (3-((R)-1-(5-chloro-4-fluoro-2-(((S)-1-methylpyrrolidin-2-yl)methoxy)-8,9-dihydro-10H-7-oxa-1,3,6,10-tetraazacyclohepta[de]naphthalen-10-yl)ethyl)pyridin-2-yl)carbamate

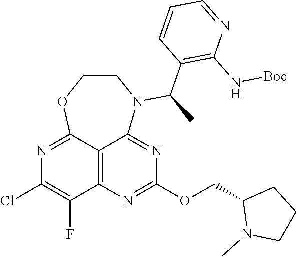 [SMILES: C[C@H](c1cccnc1NC(=O)OC(C)(C)C)N1CCOc2nc(Cl)c(F)c3nc(OC[C@@H]4CCCN4C)nc1c23]

To a solution of (S)-(1-methylpyrrolidin-2-yl)methanol (0.21 mL, 1.78 mmol) in tetrahydrofuran (4 mL) was added sodium hydride (119.0 mg, 2.97 mmol, 60% dispersion in mineral oil) at 0° C. and stirred at 25° C. for 20 minutes. Then tert-butyl N-tert-butoxycarbonyl-N-[3-[rac-(1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl]-2-pyridyl]carbamate (380.0 mg, 0.59 mmol) was added and stirred at 0° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (3:1) to afford tert-butyl (3-((R)-1-(5-chloro-4-fluoro-2-(((S)-1-methylpyrrolidin-2-yl)methoxy)-8,9-dihydro-10H-7-oxa-1,3,6,10-tetraazacyclohepta[de]naphthalen-10-yl)ethyl)pyridin-2-yl)carbamate (230.0 mg, 0.36 mmol, 62% yield) as a white solid. LC-MS: (ESI, m/z): 574.1 [M+H]⁺

### Step 2: 4-(10-((R)-1-(2-aminopyridin-3-yl)ethyl)-4-fluoro-2-(((S)-1-methylpyrrolidin-2-yl)methoxy)-9,10-dihydro-8H-7-oxa-1,3,6,10-tetraazacyclohepta[de]naphthalen-5-yl)-N,N-bis(4-methoxybenzyl)-6-methyl-5-(trifluoromethyl)pyridin-2-amine

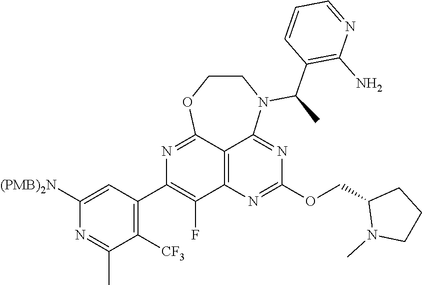

Under nitrogen, a solution of potassium phosphate (233.7 mg, 1.1 mmol), tert-butyl (3-((R)-1-(5-chloro-4-fluoro-2-(((S)-1-methylpyrrolidin-2-yl)methoxy)-8,9-dihydro-10H-7-oxa-1,3,6,10-tetraazacyclohepta[de]naphthalen-10-yl)ethyl)pyridin-2-yl)carbamate (210.0 mg, 0.37 mmol), N,N-bis(4-methoxybenzyl)-6-methyl-4-(4,4,5,5-tetramethyl-1,3,2-dioxaborolan-2-yl)-5-(trifluoromethyl)pyridin-2-amine (182.0 mg, 0.44 mmol) and mesylate[(di(1-adamantyl)-n-butylphosphine)-2-(2′-amino-1,1′-biphenyl)]palladium(II) (27.0 mg, 0.04 mmol) in 1,4-dioxane (2 mL) and water (0.20 mL) was stirred at 100° C. for 1 hour. After completion, the resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (10:1) to afford 4-(10-((R)-1-(2-aminopyridin-3-yl)ethyl)-4-fluoro-2-(((S)-1-methylpyrrolidin-2-yl)methoxy)-9,10-dihydro-8H-7-oxa-1,3,6,10-tetraazacyclohepta[de]naphthalen-5-yl)-N,N-bis(4-methoxybenzyl)-6-methyl-5-(trifluoromethyl)pyridin-2-amine (160.0 mg, 0.18 mmol, 50.2% yield) as a yellow oil. LC-MS: (ESI, m/z): 854.4 [M+H]⁺

### Step 3: 4-(10-((R)-1-(2-aminopyridin-3-yl)ethyl)-4-fluoro-2-(((S)-1-methylpyrrolidin-2-yl)methoxy)-9,10-dihydro-8H-7-oxa-1,3,6,10-tetraazacyclohepta[de]naphthalen-5-yl)-6-methyl-5-(trifluoromethyl)pyridin-2-amine

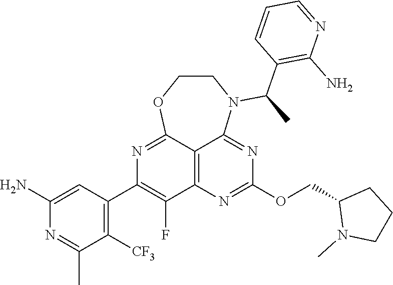 [SMILES: Cc1nc(N)cc(-c2nc3c4c(nc(OC[C@@H]5CCCN5C)nc4c2F)N([C@H](C)c2cccnc2N)CCO3)c1C(F)(F)F]

A solution of 4-(10-((R)-1-(2-aminopyridin-3-yl)ethyl)-4-fluoro-2-(((S)-1-methylpyrrolidin-2-yl)methoxy)-9,10-dihydro-8H-7-oxa-1,3,6,10-tetraazacyclohepta[de]naphthalen-5-yl)-N,N-bis(4-methoxybenzyl)-6-methyl-5-(trifluoromethyl)pyridin-2-amine (100.0 mg, 0.12 mmol) in trifluoroacetic acid (1 mL) and trifluoromethanesulfonic acid (0.1 mL) was stirred at 25° C. for 20 minutes. After completion, the reaction mixture was concentrated under vacuum, diluted with ethyl acetate, adjusted to pH>7 with sodium bicarbonate saturated solution, extracted with ethyl acetate and the organic layer was combined. The organic layer was dried over anhydrous sodium sulfate and concentrated under vacuum. The product was purified by Prep-HPLC with the following conditions: Column: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 22% B to 45% B in 10 min; Wave Length: 254 nm/220 nm; RT1(min): 10 to afford 4-(10-((R)-1-(2-aminopyridin-3-yl)ethyl)-4-fluoro-2-(((S)-1-methylpyrrolidin-2-yl)methoxy)-9,10-dihydro-8H-7-oxa-1,3,6,10-tetraazacyclohepta[de]naphthalen-5-yl)-6-methyl-5-(trifluoromethyl)pyridin-2-amine (37.8 mg, 0.06 mmol, 52.4% yield) as a white solid. LC-MS: (ESI, m/z): 614.5 [M+H]⁺

Example 627: ¹H NMR (300 MHz, DMSO-d₆, ppm) δ 7.97 (dd, J=4.9, 1.7 Hz, 1H), 7.63 (dd, J=7.6, 1.8 Hz, 1H), 6.81 (s, 2H), 6.67 (dd, J=7.5, 4.9 Hz, 1H), 6.34 (q, J=6.7 Hz, 1H), 6.22 (s, 1H), 5.63 (s, 2H), 4.47-4.36 (m, 2H), 4.35-4.22 (m, 2H), 3.70 (dd, J=16.0, 6.4 Hz, 1H), 3.38 (d, J=6.7 Hz, 1H), 2.99-2.91 (m, 1H), 2.66-2.56 (m, 1H), 2.48-2.44 (m, 3H), 2.36 (s, 3H), 2.24-2.13 (m, 1H), 2.00-1.89 (m, 1H), 1.75-1.60 (m, 3H), 1.56 (d, J=6.8 Hz, 3H). LC-MS: (ESI, m/z): 614.5 [M+H]⁺

### Example 628: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-6-methyl-5-(trifluoromethyl)pyrimidin-2-amine

### Synthetic Route

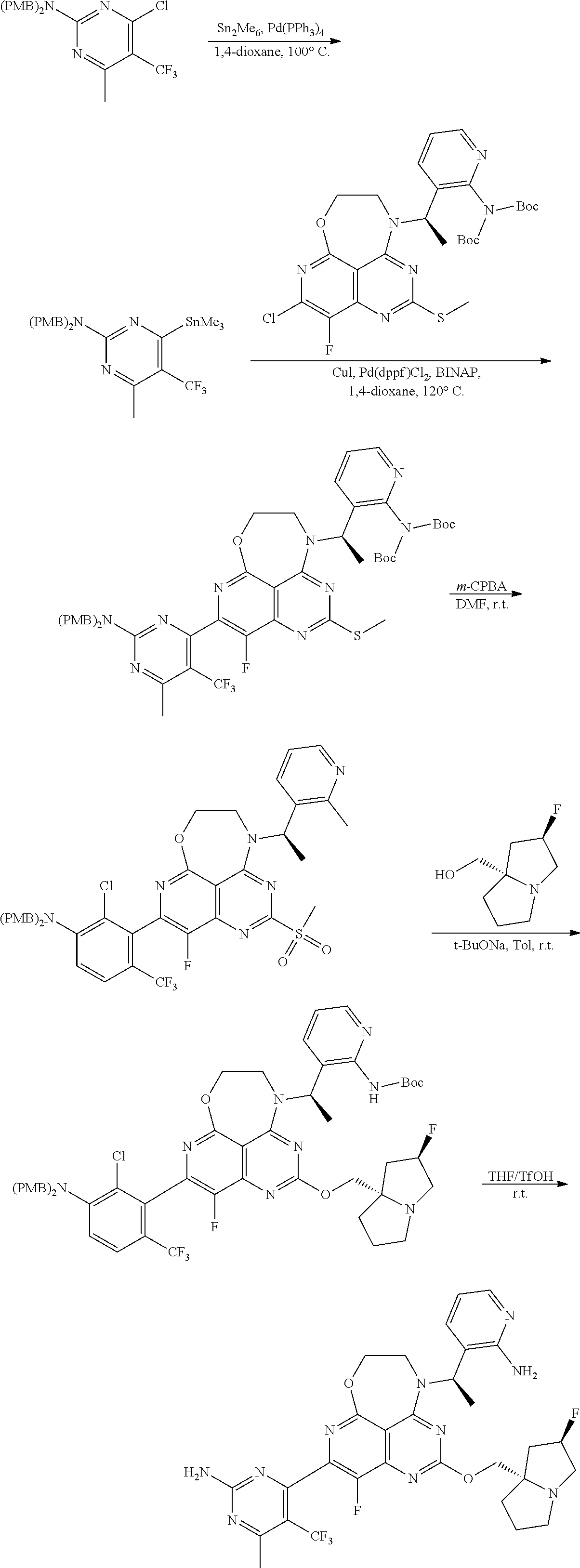

### Step 1: N,N-bis(4-methoxybenzyl)-4-methyl-5-(trifluoromethyl)-6-(trimethylstannyl)pyrimidin-2-amine

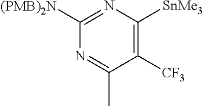

Under nitrogen, a solution of 4-chloro-N,N-bis(4-methoxybenzyl)-6-methyl-5-(trifluoromethyl)pyrimidin-2-amine (1.7 g, 3.76 mmol), hexamethyldistannane (3.69 g, 11.29 mmol) and tetrakis(triphenylphosphine)palladium (434.0 mg, 0.38 mmol) in 1,4-dioxane (17 mL) was stirred at 100° C. for 3 hours. After completion, the reaction was quenched with potassium fluoride and was diluted with water. The resulting solution was extracted with ethyl acetate and the organic layers were combined. The organic layer was dried over anhydrous sodium sulfate and concentrated under vacuum. The residue was purified by flash chromatography on C18 gel eluting with acetonitrile/water to afford N,N-bis(4-methoxybenzyl)-4-methyl-5-(trifluoromethyl)-6-(trimethylstannyl)pyrimidin-2-amine (1.5 g, 2.45 mmol, 65.3% yield) as a white solid. LC-MS: (ESI, m/z): 582.1 [M+H]⁺

### Step 2: tert-butyl N-tert-butoxycarbonyl-N-(3-(rac-(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl)-2-pyridyl)carbamate

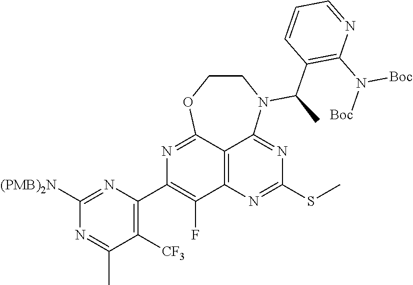

Under nitrogen, a solution of tert-butyl N-tert-butoxycarbonyl-N-(3-(rac-(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl)-2-pyridyl)carbamate (800.0 mg, 1.32 mmol) and N,N-bis(4-methoxybenzyl)-4-methyl-5-(trifluoromethyl)-6-(trimethylstannyl)pyrimidin-2-amine (1.5 g, 2.64 mmol), 1,1′-bis(diphenylphosphino)ferrocene-palladium(II)dichloride dichloromethane complex (108.0 mg, 0.13 mmol), 1.1′-binaphthyl-2.2′-diphemyl phosphine (164.0 mg, 0.26 mmol) and cuprous iodide (75.0 mg, 0.40 mmol) in 1,4-dioxane (1.5 mL) was stirred for 16 hours at 120° C. After completion, the resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (3:1) to afford tert-butyl N-tert-butoxycarbonyl-N-(3-(rac-(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl)-2-pyridyl)carbamate (450.0 mg, 423.56 mmol, 32.1% yield) as a yellow solid. LC-MS: (ESI, m/z): 988.1 [M+H]⁺

### Step 3: tert-butyl N-tert-butoxycarbonyl-N-(3-(rac-(1R)-1-(7-(2-(bis((4-methoxyphenyl)methyl)amino)-6-methyl-5-(trifluoromethyl)pyrimidin-4-yl)-6-fluoro-3-methylsulfonyl-10-oxa-2,4,8,13-tetrazatricyclo[7.4.1.05, 14]tetradeca-1,3,5(14),6,8-pentaen-13-yl)ethyl)-2-pyridyl)carbamate

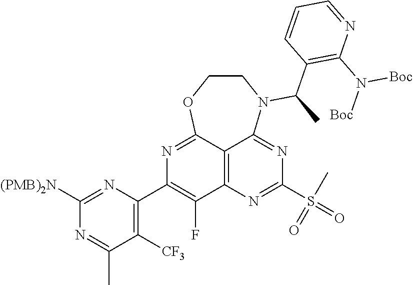

A solution of tert-butyl N-tert-butoxycarbonyl-N-(3-(rac-(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl)-2-pyridyl)carbamate (267.0 mg, 0.27 mmol) and 3-chloroperoxybenzoic acid (117.0 mg, 0.68 mmol) in dichloromethane (2.6 mL) was stirred at 25° C. for 20 minutes. After completion, the reaction was quenched with saturated sodium sulfite solution and diluted with water. The resulting solution was extracted with ethyl acetate and the organic layers were combined. The organic layer was dried over anhydrous sodium sulfate and concentrated under vacuum. The residue was purified by flash chromatography on C18 gel eluting with acetonitrile/water to afford tert-butyl N-tert-butoxycarbonyl-N-(3-(rac-(1R)-1-(7-(2-(bis((4-methoxyphenyl)methyl)amino)-6-methyl-5-(trifluoromethyl)pyrimidin-4-yl)-6-fluoro-3-methylsulfonyl-10-oxa-2,4,8,13-tetrazatricyclo[7.4.1.05, 14]tetradeca-1,3,5(14),6,8-pentaen-13-yl)ethyl)-2-pyridyl)carbamate (170.0 mg, 0.15 mmol, 58.6% yield) as a yellow solid. LC-MS: (ESI, m/z): 1020.1 [M+H]⁺

### Step 4: tert-butyl (3-((R)-1-(5-(2-(bis(4-methoxybenzyl)amino)-6-methyl-5-(trifluoromethyl)pyrimidin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate

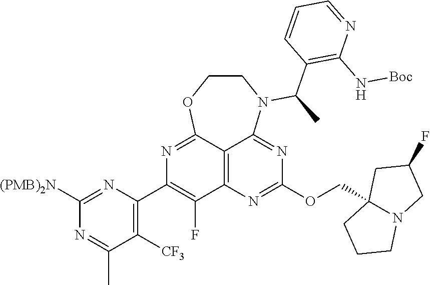

To a solution of ((2R,7aS)-2-fluorotetrahydro-1H-pyrrolizin-7a(5H)-yl)methanol (80.0 mg, 0.50 mmol) in tetrahydrofuran (1.7 mL) was added sodium hydride (33.0 mg, 0.83 mmol, 60% dispersion in mineral oil) at 0° C. and stirred at 25° C. for 20 minutes. Then tert-butyl N-tert-butoxycarbonyl-N-[3-[rac-(1R)-1-[7-[2-[bis[(4-methoxyphenyl)methyl]amino]-6-methyl-5-(trifluoromethyl)pyrimidin-4-yl]-6-fluoro-3-methylsulfonyl-10-oxa-2,4,8,13-tetrazatricyclo[7.4.1.05, 14]tetradeca-1,3,5(14),6,8-pentaen-13-yl]ethyl]-2-pyridyl]carbamate (170.0 mg, 0.17 mmol) was added and stirred at 25° C. for 1 hour. After completion, the reaction was quenched saturated ammonium chloride solution and diluted with water. The resulting solution was extracted with ethyl acetate and the organic layers were combined. The organic layer was dried over anhydrous sodium sulfate and concentrated under vacuum. The residue was purified by flash chromatography on silica gel eluting with petroleum ether/ethyl acetate (3:1) to afford tert-butyl (3-((R)-1-(5-(2-(bis(4-methoxybenzyl)amino)-6-methyl-5-(trifluoromethyl)pyrimidin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (90.0 mg, 0.08 mmol, 51.4% yield) as a white solid. LC-MS: (ESI, m/z): 999.1 [M+H]⁺

### Step 5: 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-6-methyl-5-(trifluoromethyl)pyrimidin-2-amine

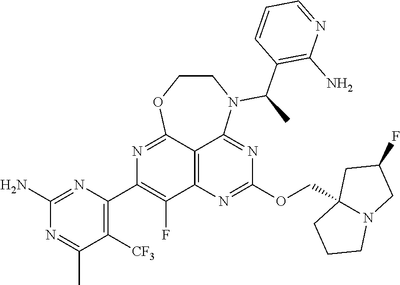 [SMILES: Cc1nc(N)nc(-c2nc3c4c(nc(OC[C@@]56CCCN5C[C@H](F)C6)nc4c2F)N([C@H](C)c2cccnc2N)CCO3)c1C(F)(F)F]

A solution of tert-butyl (3-((R)-1-(5-(2-(bis(4-methoxybenzyl)amino)-6-methyl-5-(trifluoromethyl)pyrimidin-4-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (80.0 mg, 0.08 mmol) in trifluoroacetic acid (0.8 mL) and trifluoromethanesulfonic acid (0.08 mL) was stirred at 25° C. for 20 minutes. After completion, the reaction mixture was concentrated under vacuum, diluted with ethyl acetate, adjusted to pH>7 with sodium bicarbonate saturated solution, extracted with ethyl acetate and the organic layer was combined. The organic layer was dried over anhydrous sodium sulfate and concentrated under vacuum. The product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 59% B to 83% B in 9 min; Wave Length: 254 nm/220 nm; RT1(min): 7.9 to afford 4-(10-((R)-1-(2-aminopyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-6-methyl-5-(trifluoromethyl)pyrimidin-2-amine (20.7 mg, 0.03 mmol, 39% yield) as a white solid. LC-MS: (ESI, m/z): 659.2 [M+H]⁺

Example 628: ¹H NMR (500 MHz, DMSO-d₆, ppm) δ 7.97 (dd, J=4.9, 1.7 Hz, 1H), 7.70-7.52 (m, 3H), 6.67 (dd, J=7.5, 4.9 Hz, 1H), 6.31 (q, J=6.8 Hz, 1H), 5.69 (s, 2H), 5.29 (d, J=53.6 Hz, 1H), 4.43 (dd, J=12.7, 6.7 Hz, 1H), 4.30 (dd, J=12.6, 6.6 Hz, 1H), 4.17-4.08 (m, 2H), 3.71 (dd, J=16.0, 6.6 Hz, 1H), 3.41-3.36 (m, 1H), 3.13-3.05 (m, 2H), 3.04-2.99 (m, 1H), 2.85-2.79 (m, 1H), 2.51 (s, 3H), 2.18-1.99 (m, 3H), 1.89-1.74 (m, 3H), 1.56 (d, J=6.8 Hz, 3H). LC-MS: (ESI, m/z): 659.2 [M+H]⁺.

### Example 629a: 3-((R)-1-((R)-5-(3-amino-2,4-difluoro-5-methyl-6-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Example 629b: 3-((R)-1-((S)-5-(3-amino-2,4-difluoro-5-methyl-6-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

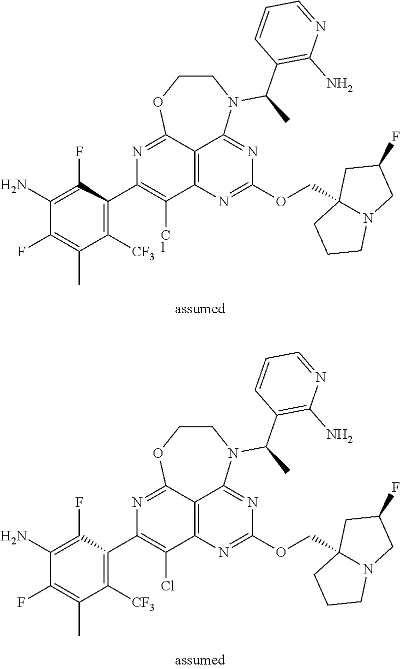

### Synthetic Route

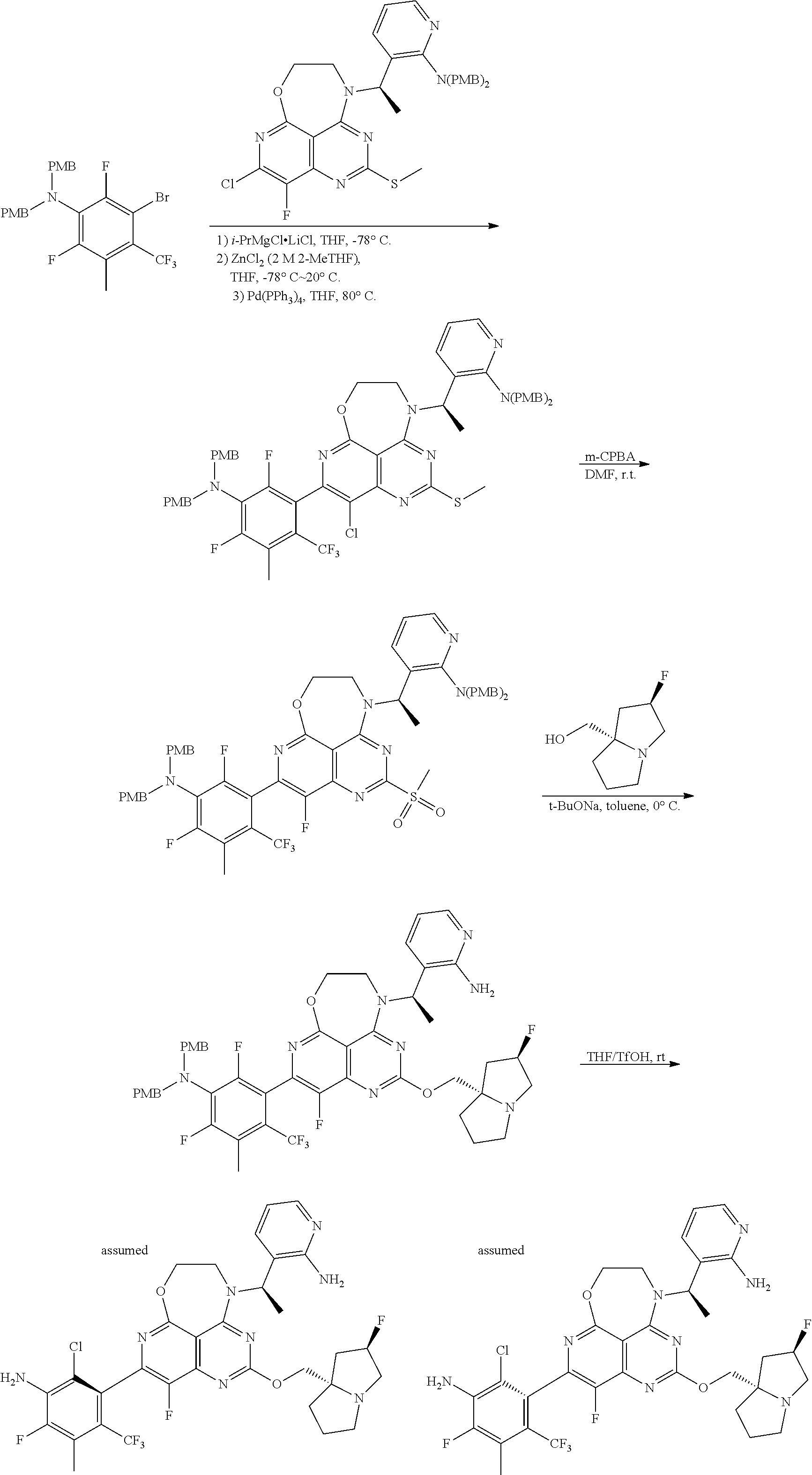

### Step 1: 3-((1R)-1-(5-(3-(bis(4-methoxybenzyl)amino)-2,4-difluoro-5-methyl-6-(trifluoromethyl)phenyl)-4-chloro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine

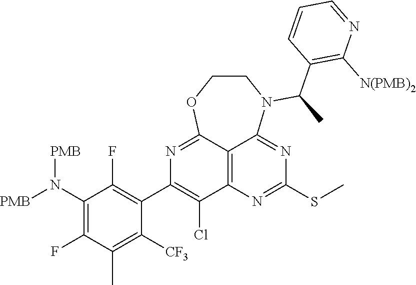

Under nitrogen, to a solution of 3-bromo-2,6-difluoro-N,N-bis(4-methoxybenzyl)-5-methyl-4-(trifluoromethyl)aniline (700.0 mg, 1.32 mmol) in tetrahydrofuran (7 mL) was added isopropyl magnesium chloride lithium chloride (1.6 mL, 2.09 mmol, 1.3 M in tetrahydrofuran), the mixture was stirred at −78° C. for 30 minutes. Then zinc chloride (1.9 mL, 3.92 mmol, 2 M in 2-methyltetrahydrofuran) was added at −78° C. and stirred at 25° C. for 30 minutes. The resulting mixture was added into a solution of (R)-3-(1-(4,5-dichloro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (875.0 mg, 1.32 mmol) and tetrakis(triphenylphosphine)palladium (153.0 mg, 0.13 mmol) in tetrahydrofuran (6 mL) and stirred at 80° C. for 1 hour under nitrogen. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate to afford 3-((1R)-1-(5-(3-(bis(4-methoxybenzyl)amino)-2,4-difluoro-5-methyl-6-(trifluoromethyl)phenyl)-4-chloro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (300.0 mg, 0.27 mmol, 18.8% yield) as a yellow solid. LC-MS: (ESI, m/z): 1078.6 [M+H]⁺

### Step 2: 3-((1R)-1-(5-(3-(bis(4-methoxybenzyl)amino)-2,4-difluoro-5-methyl-6-(trifluoromethyl)phenyl)-4-chloro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine

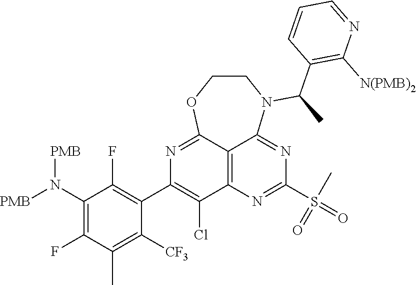

A solution of 3-((1R)-1-(5-(3-(bis(4-methoxybenzyl)amino)-2,4-difluoro-5-methyl-6-(trifluoromethyl)phenyl)-4-chloro-2-(methylthio)-8,9-dihydro-10H-7-oxa-1,3,6,10-tetraazacyclohepta[de]naphthalen-10-yl)ethyl)-N,N-bis(4-methoxybenzyl)pyridin-2-amine (300.0 mg, 0.27 mmol) and m-chloro-peroxybenzoic acid (144.0 mg, 0.83 mmol) in dichloromethane (1.5 mL) was stirred at 25° C. for 20 minutes. After completion, the reaction was quenched with saturated sodium sulfite solution. The reaction mixture was diluted with water. The resulting solution was extracted with ethyl acetate and the organic layers were combined. The organic layer was dried over anhydrous sodium sulfate and concentrated under vacuum. The residue was purified by flash chromatography on C18 gel eluting with acetonitrile/water to afford 3-((1R)-1-(5-(3-(bis(4-methoxybenzyl)amino)-2,4-difluoro-5-methyl-6-(trifluoromethyl)phenyl)-4-chloro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine (230.0 mg, 0.20 mmol, 50.9% yield) as a yellow solid. LC-MS: (ESI, m/z):1110.6 [M+H]⁺.

### Step 3: 3-((1R)-1-(5-(3-(bis(4-methoxybenzyl)amino)-2,4-difluoro-5-methyl-6-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine

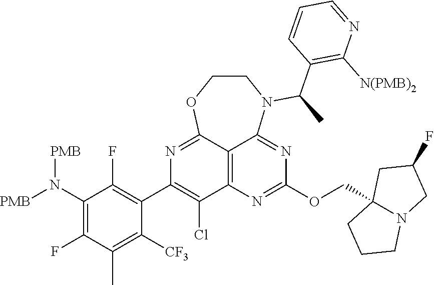

A solution of ((2R,7aS)-2-fluorotetrahydro-1H-pyrrolizin-7a(5H)-yl)methanol (62.0 mg, 0.39 mmol) and sodium tert-butoxide (76.0 mg, 0.79 mmol) in toluene (2.2 mL) was stirred at 0° C. for 10 minutes. Then 3-((1R)-1-(5-(3-(bis(4-methoxybenzyl)amino)-2,4-difluoro-5-methyl-6-(trifluoromethyl)phenyl)-4-chloro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyridin-2-amine (220.0 mg, 0.20 mmol) was added and stirred at 0° C. for 10 minutes. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (10:1) to afford 3-((1R)-1-(5-(3-(bis(4-methoxybenzyl)amino)-2,4-difluoro-5-methyl-6-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (120.0 mg, 0.10 mmol, 49.9% yield) as a yellow solid. LC-MS: (ESI, m/z):1189.7 [M+H]⁺

### Step 4: 3-((R)-1-((R)-5-(3-amino-2,4-difluoro-5-methyl-6-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine & 3-((R)-1-((S)-5-(3-amino-2,4-difluoro-5-methyl-6-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

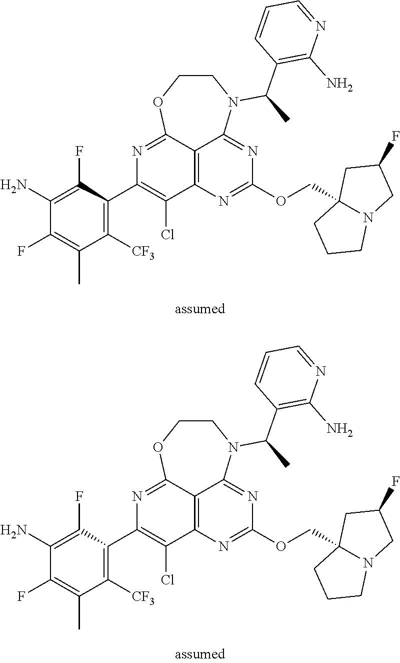

A solution of 3-((1R)-1-(5-(3-(bis(4-methoxybenzyl)amino)-2,4-difluoro-5-methyl-6-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (120.0 mg, 0.10 mmol) in trifluoroacetic acid (1 mL) and trifluoromethanesulfonic acid (0.1 ml) was stirred at 25° C. for 20 minutes. After completion, the reaction mixture was concentrated under vacuum, diluted with ethyl acetate, adjusted to pH>7 with sodium bicarbonate saturated solution, extracted with ethyl acetate and the organic layer was combined. The organic layer was dried over anhydrous sodium sulfate and concentrated under vacuum. The product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 37% B to 67% B in 8 min; Wave Length: 254 nm/220 nm to afford 3-((R)-1-((R)-5-(3-amino-2,4-difluoro-5-methyl-6-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (7.7 mg, 0.01 mmol, 8.6% yield) as a white solid and 3-((R)-1-((S)-5-(3-amino-2,4-difluoro-5-methyl-6-(trifluoromethyl)phenyl)-4-chloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (9.0 mg, 0.01 mmol, 10% yield) as a white solid.

Example 629a: ¹H NMR (300 MHz, Methanol-d₄, ppm) δ 7.97 (dd, J=5.1, 1.6 Hz, 1H), 7.74 (d, J=7.5 Hz, 1H), 6.80-6.72 (m, 1H), 6.61 (q, J=6.9 Hz, 1H), 5.30 (d, J=54.0 Hz, 1H), 4.52-4.28 (m, 4H), 3.75 (dd, J=16.2, 6.3 Hz, 1H), 3.52 (dd, J=16.0, 6.2 Hz, 1H), 3.29-3.16 (m, 3H), 3.08-2.96 (m, 1H), 2.37 (s, 3H), 2.31-2.11 (m, 3H), 2.07-1.84 (m, 3H), 1.65 (d, J=6.8 Hz, 3H). LC-MS: (ESI, m/z):709.2 [M+H]⁺

Example 629b: ¹H NMR (300 MHz, Methanol-d₄, ppm) δ 7.98-7.90 (m, 1H), 7.78-7.68 (m, 1H), 6.80-6.72 (m, 1H), 6.61 (q, J=6.8 Hz, 1H), 5.29 (d, J=54.0 Hz, 1H), 4.52-4.26 (m, 4H), 3.74 (dd, J=16.0, 6.4 Hz, 1H), 3.54 (dd, J=16.1, 6.2 Hz, 1H), 3.29-3.15 (m, 3H), 3.07-2.94 (m, 1H), 2.38 (s, 3H), 2.30-2.11 (m, 3H), 2.05-1.83 (m, 3H), 1.65 (d, J=6.8 Hz, 3H). LC-MS: (ESI, m/z):709.2 [M+H]⁺

### Example 630: 3-((R)-1-(5-(6-amino-5-fluoro-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine

### Synthetic Route

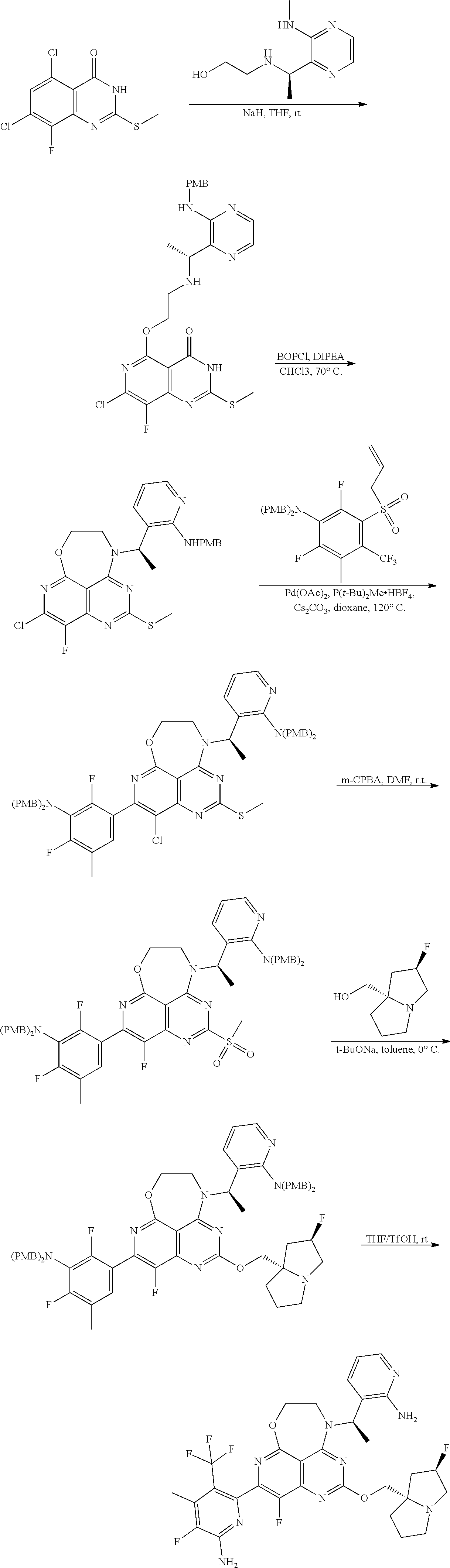

### Step 1: (R)-7-Chloro-8-fluoro-5-(2-((1-(3-((4-methoxybenzyl)amino)pyrazin-2-yl)ethyl)amino)ethoxy)-2-(methylthio)pyrido[4,3-d]pyrimidin-4(3H)-one

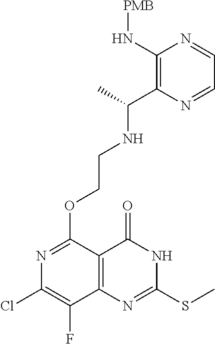

Sodium hydride (2.9 g, 72.5 mmol, 60% in mineral oil) was added to a solution of 2-[[(1R)-1-[3-[(4-methoxyphenyl)methylamino]pyrazin-2-yl]ethyl]amino]ethanol (6.5 g, 21.4 mmol) in tetrahydrofuran (50.0 mL) under nitrogen atmosphere at 0° C., and the mixture was stirred at 0° C. for 0.5 hours. Then the mixture was added to a solution of 5,7-dichloro-8-fluoro-2-methylsulfanyl-3H-pyrido[4,3-d]pyrimidin-4-one (5.0 g, 17.8 mmol) in tetrahydrofuran (50.0 mL) and stirred at room temperature for 1 hour. After completion, the reaction was quenched with sat. ammonium chloride aq. and extracted by ethyl acetate. The organic layer was washed by brine and dried over anhydrous sodium sulfate. After concentration under vacuum, the residue was used in next step without purification. LC-MS: (ESI, m/z): 546.2 [M+H]⁺

### Step 2: (R)-3-(1-(5-Chloro-4-fluoro-2-(methylthio)-8,9-dihydro-10H-7-oxa-1,3,6,10-tetraazacyclohepta[de]naphthalen-10-yl)ethyl)-N-(4-methoxybenzyl)pyrazin-2-amine

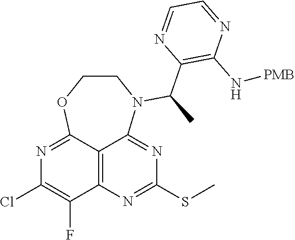

A mixture of 7-chloro-8-fluoro-5-[2-[[(1R)-1-[3-[(4-methoxyphenyl)methylamino]pyrazin-2-yl]ethyl]amino]ethoxy]-2-methylsulfanyl-3H-pyrido[4,3-d]pyrimidin-4-one (9.8 g, crude), bis(2-oxo-3-oxazolidinyl)phosphinic chloride (6.8 g, 26.9 mmol) and N,N-diisopropylethylamine (9.4 mL, 53.8 mmol) in chloroform (80 mL) was stirred at 70° C. for 1 hour. After completion, the reaction mixture was diluted with water and extracted with dichloromethane. The combined organic layers were washed with brine, dried over anhydrous sodium sulfate and concentrated in vacuum. The residue was purified by flash chromatography on silica gel eluting with petroleum ether/ethyl acetate (4:1) to afford the titled compound (7.0 g, 13.3 mmol, 74% yield) as a light yellow solid. LC-MS: (ESI, m/z): 528.0 [M+H]⁺

### Step 3: (R)-3-(1-(5-(6-(bis(4-methoxybenzyl)amino)-5-fluoro-4-methyl-3-(trifluoromethyl)pyridin-2-yl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyrazin-2-amine

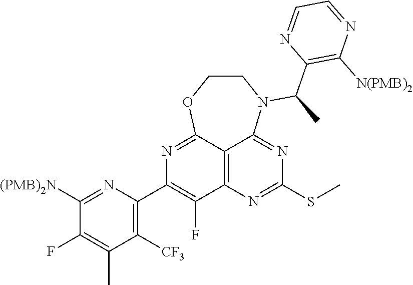

Under nitrogen, a solution of palladium (II) acetate (10.0 mg, 0.06 mmol), 6-(allylsulfonyl)-3-fluoro-N,N-bis(4-methoxybenzyl)-4-methyl-5-(trifluoromethyl)pyridin-2-amine (827.0 mg, 1.53 mmol), (R)-3-(1-(5-chloro-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyrazin-2-amine (500.0 mg, 0.77 mmol) and di-t-butylmethylphosphonium tetrafluoroborate (38.0 mg, 0.15 mmol) and cesium carbonate (750 mg, 2.31 mmol) in 1,4-dioxane (5 mL) was stirred at 120° C. for 3 hours. After completion, the resulting solution was diluted with water and extracted with ethyl acetate. The organic layers were washed with brine, dried over anhydrous sodium sulfate and concentrated. The resulting residue was purified by reverse phase chromatography (acetonitrile/0.1% ammonium bicarbonate in water) to afford (R)-3-(1-(5-(6-(bis(4-methoxybenzyl)amino)-5-fluoro-4-methyl-3-(trifluoromethyl)pyridin-2-yl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyrazin-2-amine (150.0 mg, 0.14 mmol, 17.7% yield) as a yellow solid. LC-MS: (ESI, m/z): 1046.4 [M+H]⁺

### Step 4: (R)-3-(1-(5-(6-(bis(4-methoxybenzyl)amino)-5-fluoro-4-methyl-3-(trifluoromethyl)pyridin-2-yl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyrazin-2-amine

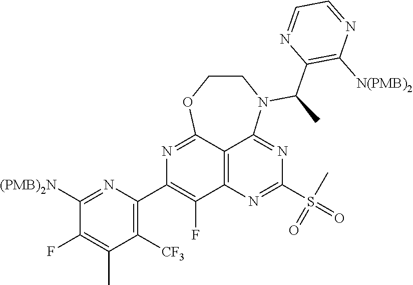

A solution of (R)-3-(1-(5-(6-(bis(4-methoxybenzyl)amino)-5-fluoro-4-methyl-3-(trifluoromethyl)pyridin-2-yl)-4-fluoro-2-(methylthio)-8,9-dihydro-10H-7-oxa-1,3,6,10-tetraazacyclohepta[de]naphthalen-10-yl)ethyl)-N,N-bis(4-methoxybenzyl)pyrazin-2-amine (150.0 mg, 0.14 mmol) and 3-chloroperoxybenzoic acid (62.0 mg, 0.36 mmol) in dichloromethane (1.5 mL) was stirred at 25° C. for 20 minutes. After completion, the reaction was quenched with saturated sodium sulfite solution. The reaction mixture was diluted with water. The resulting solution was extracted with ethyl acetate and the organic layers were combined. The organic layer was dried over anhydrous sodium sulfate and concentrated under vacuum. The resulting residue was purified by reverse phase chromatography (acetonitrile/0.1% ammonium bicarbonate in water) to afford (R)-3-(1-(5-(6-(bis(4-methoxybenzyl)amino)-5-fluoro-4-methyl-3-(trifluoromethyl)pyridin-2-yl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyrazin-2-amine (110.0 mg, 0.10 mmol, 66.9% yield) as a yellow solid. LC-MS: (ESI, m/z): 1078.1 [M+H]⁺

### Step 5: 3-((R)-1-(5-(6-(bis(4-methoxybenzyl)amino)-5-fluoro-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine

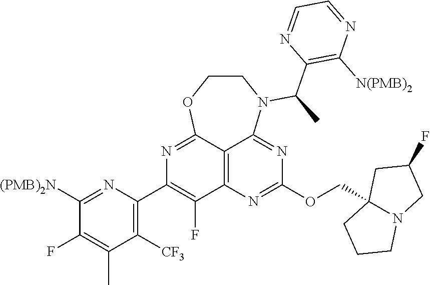

A solution of ((2R,7aS)-2-fluorotetrahydro-1H-pyrrolizin-7a(5H)-yl)methanol (32.0 mg, 0.20 mmol) and sodium tert-butoxide (29.0 mg, 0.31 mmol) in toluene (1.1 mL) was stirred at 0° C. for 10 minutes. Then (R)-3-(1-(5-(6-(bis(4-methoxybenzyl)amino)-5-fluoro-4-methyl-3-(trifluoromethyl)pyridin-2-yl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyrazin-2-amine (110.0 mg, 0.10 mmol) was added and stirred at 0° C. for 10 minutes. After completion, the resulting solution was diluted with water and extracted with ethyl acetate. The organic layers were washed with brine, dried over anhydrous sodium sulfate and concentrated. The resulting residue was purified by reverse phase chromatography (acetonitrile/0.1% ammonium bicarbonate in water) to afford 3-((R)-1-(5-(6-(bis(4-methoxybenzyl)amino)-5-fluoro-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine (30.0 mg, 0.02 mmol, 25.6% yield) as a yellow solid. LC-MS: (ESI, m/z): 1157.2 [M+H]⁺

### Step 6: 3-((R)-1-(5-(6-amino-5-fluoro-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine

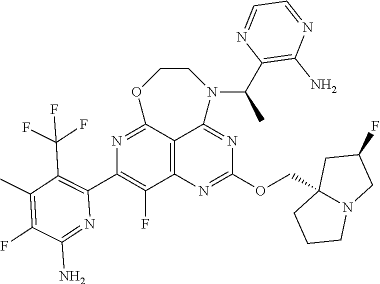 [SMILES: Cc1c(F)c(N)nc(-c2nc3c4c(nc(OC[C@@]56CCCN5C[C@H](F)C6)nc4c2F)N([C@H](C)c2nccnc2N)CCO3)c1C(F)(F)F]

A solution of 3-((R)-1-(5-(6-(bis(4-methoxybenzyl)amino)-5-fluoro-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyrazin-2-amine (30.0 mg, 0.02 mmol) in trifluoroacetic acid (0.5 mL) and trifluoromethanesulfonic acid (0.05 mL) was stirred at 25° C. for 20 minutes. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 59% B to 83% B in 9 min; Wave Length: 254 nm/220 nm to afford 3-((R)-1-(5-(6-amino-5-fluoro-4-methyl-3-(trifluoromethyl)pyridin-2-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyrazin-2-amine (7.5 mg, 0.01 mmol, 42.3% yield) as a white solid. LC-MS: (ESI, m/z): 677.2 [M+H]⁺

Example 630: ¹H NMR (500 MHz, Methanol-d₄, ppm) δ 7.93 (dd, J=2.8, 1.1 Hz, 1H), 7.85 (dd, J=2.8, 0.9 Hz, 1H), 6.55 (q, J=6.8 Hz, 1H), 5.32 (d, J=54.3 Hz, 1H), 4.56 (dd, J=12.9, 6.2 Hz, 1H), 4.44 (dd, J=12.9, 6.7 Hz, 1H), 4.31-4.19 (m, 2H), 4.00-3.85 (m, 2H), 3.29-3.13 (m, 3H), 3.03-2.97 (m, 1H), 2.41-2.29 (m, 3H), 2.27-2.08 (m, 3H), 2.01-1.85 (m, 3H), 1.67 (d, J=6.7 Hz, 3H). LC-MS: (ESI, m/z): 677.2 [M+H]⁺

### Example 631: 6-(10-((R)-1-(2-amino-5-fluoropyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-4-methyl-5-(trifluoromethyl)pyridin-2-amine

### Synthetic Route

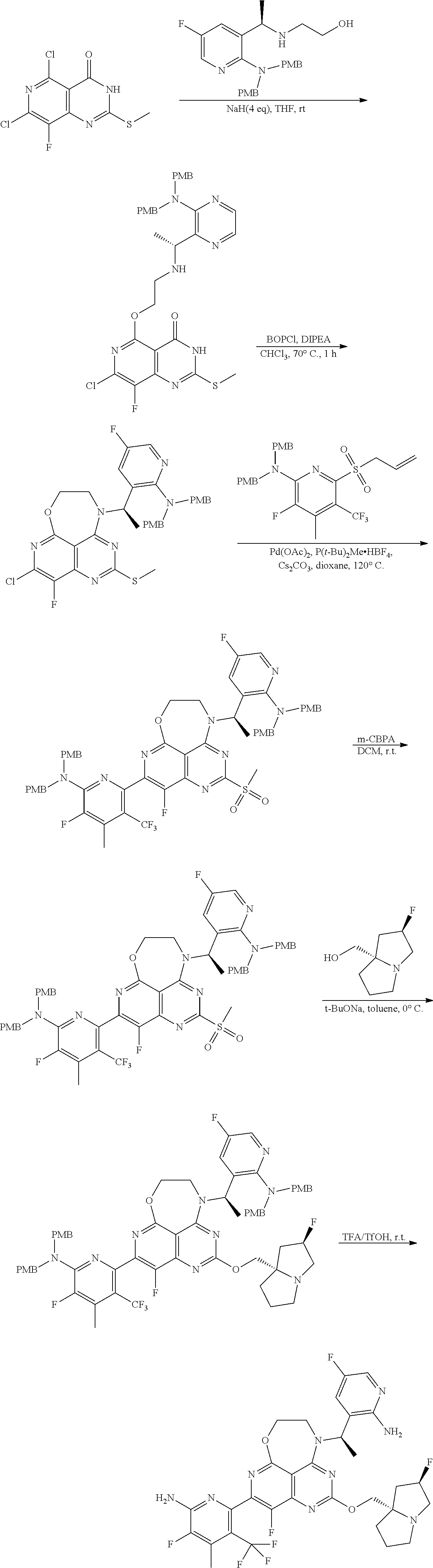

### Step 1: (R)-5-(2-((1-(2-(bis(4-methoxybenzyl)amino)-5-fluoropyridin-3-yl)ethyl)amino)ethoxy)-7-chloro-8-fluoro-2-(methylthio)pyrido[4,3-d]pyrimidin-4(3H)-one

To a solution of (R)-2-((1-(2-(bis(4-methoxybenzyl)amino)-5-fluoropyridin-3-yl)ethyl)amino)ethan-1-ol (2.99 g, 6.81 mmol) in tetrahydrofuran (17 mL) was added sodium hydride (990.4 mg, 24.76 mmol, 60% dispersion in mineral oil) at 0° C., the mixture was stirred at 25° C. for 15 minutes. Then 5,7-dichloro-8-fluoro-2-(methylthio)pyrido[4,3-d]pyrimidin-4(3H)-one (1.73 g, 6.19 mmol) was added and stirred at 25° C. for 1.5 hours. After completion, the reaction was quenched with saturated ammonium chloride solution. The resulting solution was diluted with water and extracted with ethyl acetate. The organic layers were combined, washed with brine, dried over anhydrous sodium sulfate and concentrated. The crude product would be directly used in the next step without purification. LC-MS: (ESI, m/z): 683.2 [M+H]⁺

### Step 2: (R)-3-(1-(5-chloro-4-fluoro-2-(methylthio)-8,9-dihydro-10H-7-oxa-1,3,6,10-tetraazacyclohepta[de]naphthalen-10-yl)ethyl)-5-fluoro-N,N-bis(4-methoxybenzyl)pyridin-2-amine

A solution of (R)-5-(2-((1-(2-(bis(4-methoxybenzyl)amino)-5-fluoropyridin-3-yl)ethyl)amino)ethoxy)-7-chloro-8-fluoro-2-(methylthio)pyrido[4,3-d]pyrimidin-4(3H)-one (3.1 g, 4.54 mmol), bis(2-oxo-3-oxazolidinyl)phosphinic chloride (1.38 g, 5.45 mmol), and N,N-diisopropylethylamine (1.76 g, 13.62 mmol) in chloroform (31 mL) was stirred at 70° C. for 1 hour. After completion, the resulting solution was diluted with water and extracted with ethyl acetate. The organic layers were washed with brine, dried over anhydrous sodium sulfate and concentrated. The resulting residue was purified by reverse phase chromatography (acetonitrile/0.1% ammonium bicarbonate in water) to afford (R)-3-(1-(5-chloro-4-fluoro-2-(methylthio)-8,9-dihydro-10H-7-oxa-1,3,6,10-tetraazacyclohepta[de]naphthalen-10-yl)ethyl)-5-fluoro-N,N-bis(4-methoxybenzyl)pyridin-2-amine (1.4 g, 2.04 mmol, 45% yield) as a yellow solid. LC-MS: (ESI, m/z): 665.2 [M+H]⁺

### Step 3: (R)-6-(10-(1-(2-(bis(4-methoxybenzyl)amino)-5-fluoropyridin-3-yl)ethyl)-4-fluoro-2-(methylthio)-9,10-dihydro-8H-7-oxa-1,3,6,10-tetraazacyclohepta[de]naphthalen-5-yl)-3-fluoro-N,N-bis(4-methoxybenzyl)-4-methyl-5-(trifluoromethyl)pyridin-2-amine

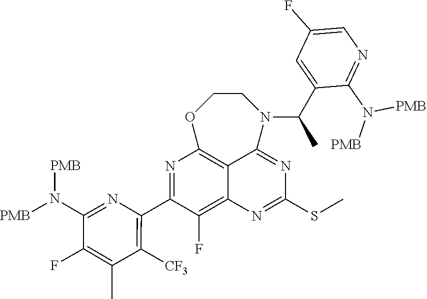

Under nitrogen, a solution of (R)-3-(1-(5-chloro-4-fluoro-2-(methylthio)-8,9-dihydro-10H-7-oxa-1,3,6,10-tetraazacyclohepta[de]naphthalen-10-yl)ethyl)-5-fluoro-N,N-bis(4-methoxybenzyl)pyridin-2-amine (800.0 mg, 1.2 mmol), 6-(allylsulfonyl)-3-fluoro-N,N-bis(4-methoxybenzyl)-4-methyl-5-(trifluoromethyl)pyridin-2-amine (1.3 g, 2.41 mmol), palladium (II) acetate (20.0 mg, 0.12 mmol) and di-t-butylmethylphosphonium tetrafluoroborate (60.0 mg, 0.24 mmol) and cesium carbonate (1.1 g, 3.61 mmol) was stirred for 4 hours at 120° C. After completion, the reaction mixture was diluted with water. The resulting solution was extracted with ethyl acetate and the organic layers were combined. The organic layer was dried over anhydrous sodium sulfate and concentrated under vacuum. The residue was purified by flash chromatography on silica gel eluting with petroleum ether/ethyl acetate (3:1) to afford (R)-6-(10-(1-(2-(bis(4-methoxybenzyl)amino)-5-fluoropyridin-3-yl)ethyl)-4-fluoro-2-(methylthio)-9,10-dihydro-8H-7-oxa-1,3,6,10-tetraazacyclohepta[de]naphthalen-5-yl)-3-fluoro-N,N-bis(4-methoxybenzyl)-4-methyl-5-(trifluoromethyl)pyridin-2-amine (300.0 mg, 0.25 mmol, 21.1% yield) as a yellow solid. LC-MS: (ESI, m/z): 1063.1 [M+H]⁺

### Step 4: (R)-6-(10-(1-(2-(bis(4-methoxybenzyl)amino)-5-fluoropyridin-3-yl)ethyl)-4-fluoro-2-(methylsulfonyl)-9,10-dihydro-8H-7-oxa-1,3,6,10-tetraazacyclohepta[de]naphthalen-5-yl)-3-fluoro-N,N-bis(4-methoxybenzyl)-4-methyl-5-(trifluoromethyl)pyridin-2-amine

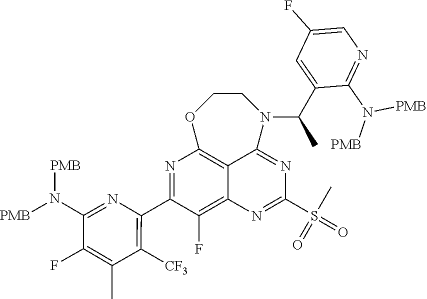

A solution of (R)-6-(10-(1-(2-(bis(4-methoxybenzyl)amino)-5-fluoropyridin-3-yl)ethyl)-4-fluoro-2-(methylthio)-9,10-dihydro-8H-7-oxa-1,3,6,10-tetraazacyclohepta[de]naphthalen-5-yl)-3-fluoro-N,N-bis(4-methoxybenzyl)-4-methyl-5-(trifluoromethyl)pyridin-2-amine (290.0 mg, 0.27 mmol) and 3-chloroperoxybenzoic acid (118.0 mg, 0.68 mmol) in dichloromethane (3 mL) was stirred at 25° C. for 20 minutes. After completion, the reaction was quenched with saturated sodium sulfite solution. The reaction mixture was diluted with water. The resulting solution was extracted with ethyl acetate and the organic layers were combined. The organic layer was dried over anhydrous sodium sulfate and concentrated under vacuum. The resulting residue was purified by reverse phase chromatography (acetonitrile/0.1% ammonium bicarbonate in water) to afford (R)-6-(10-(1-(2-(bis(4-methoxybenzyl)amino)-5-fluoropyridin-3-yl)ethyl)-4-fluoro-2-(methylsulfonyl)-9,10-dihydro-8H-7-oxa-1,3,6,10-tetraazacyclohepta[de]naphthalen-5-yl)-3-fluoro-N,N-bis(4-methoxybenzyl)-4-methyl-5-(trifluoromethyl)pyridin-2-amine (210.0 mg, 0.19 mmol, 62.6% yield) as a yellow solid. LC-MS: (ESI, m/z): 1095.1 [M+H]⁺

### Step 5: 6-(10-((R)-1-(2-(bis(4-methoxybenzyl)amino)-5-fluoropyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-N,N-bis(4-methoxybenzyl)-4-methyl-5-(trifluoromethyl)pyridin-2-amine

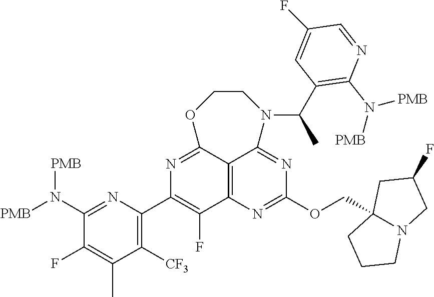

A solution of ((2R,7aS)-2-fluorotetrahydro-1H-pyrrolizin-7a(5H)-yl)methanol (59.0 mg, 0.37 mmol) and sodium tert-butoxide (53.0 mg, 0.56 mmol) in toluene (2 mL) was stirred at 0° C. for 10 minutes. Then (R)-6-(10-(1-(2-(bis(4-methoxybenzyl)amino)-5-fluoropyridin-3-yl)ethyl)-4-fluoro-2-(methylsulfonyl)-9,10-dihydro-8H-7-oxa-1,3,6,10-tetraazacyclohepta[de]naphthalen-5-yl)-3-fluoro-N,N-bis(4-methoxybenzyl)-4-methyl-5-(trifluoromethyl)pyridin-2-amine (200.0 mg, 0.19 mmol) was added and stirred at 0° C. for 10 minutes. After completion, the reaction mixture was diluted with water. The resulting solution was extracted with ethyl acetate and the organic layers were combined. The organic layer was dried over anhydrous sodium sulfate and concentrated under vacuum. The resulting residue was purified by reverse phase chromatography (acetonitrile/0.1% ammonium bicarbonate in water) to afford 6-(10-((R)-1-(2-(bis(4-methoxybenzyl)amino)-5-fluoropyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-N,N-bis(4-methoxybenzyl)-4-methyl-5-(trifluoromethyl)pyridin-2-amine (130.0 mg, 0.11 mmol, 66.3% yield) as a yellow solid. LC-MS: (ESI, m/z): 1174.2 [M+H]⁺

### Step 6: 6-(10-((R)-1-(2-amino-5-fluoropyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-4-methyl-5-(trifluoromethyl)pyridin-2-amine

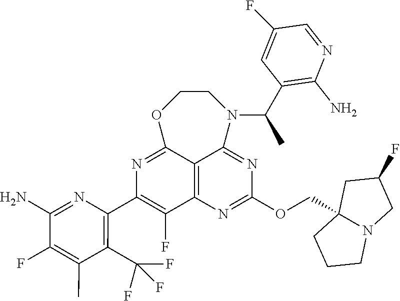 [SMILES: Cc1c(F)c(N)nc(-c2nc3c4c(nc(OC[C@@]56CCCN5C[C@H](F)C6)nc4c2F)N([C@H](C)c2cc(F)cnc2N)CCO3)c1C(F)(F)F]

A solution of 6-(10-((R)-1-(2-(bis(4-methoxybenzyl)amino)-5-fluoropyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-N,N-bis(4-methoxybenzyl)-4-methyl-5-(trifluoromethyl)pyridin-2-amine (130.0 mg, 0.11 mmol) in trifluoroacetic acid (1.3 mL) and trifluoromethanesulfonic acid (0.13 mL) was stirred at 25° C. for 20 minutes. After completion, the reaction mixture was concentrated under vacuum, diluted with ethyl acetate, adjusted to pH>7 with sodium bicarbonate saturated solution, extracted with ethyl acetate and the organic layer was combined. The organic layer was dried over anhydrous sodium sulfate and concentrated under vacuum. The product was purified by Prep-HPLC with the following conditions: Column: XSelect CSH Fluoro Phenyl 30*150 mm, 5 μm nm; Mobile Phase A: Water(10 mmol/L NH₄HCO₃), Mobile Phase B: ACN; Flow rate: 60 mL/min; Gradient: 35% B to 60% B in 9 min; Wave Length: 254 nm/220 nm to afford 6-(10-((R)-1-(2-amino-5-fluoropyridin-3-yl)ethyl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-3-fluoro-4-methyl-5-(trifluoromethyl)pyridin-2-amine (27.7 mg, 0.04 mmol, 34.9% yield) as a white solid. LC-MS: (ESI, m/z): 694.4 [M+H]⁺

Example 631: ¹H NMR (500 MHz, DMSO-d₆, ppm) δ 7.96 (d, J=2.8 Hz, 1H), 7.59 (dd, J=9.6, 2.9 Hz, 1H), 7.15 (s, 2H), 6.29 (q, J=6.9 Hz, 1H), 5.67 (s, 2H), 5.29 (d, J=53.5 Hz, 1H), 4.43 (dd, J=12.8, 6.7 Hz, 1H), 4.32 (dd, J=12.7, 6.5 Hz, 1H), 4.11 (s, 2H), 3.75 (dd, J=15.9, 6.5 Hz, 1H), 3.42 (dd, J=15.9, 6.7 Hz, 1H), 3.12-3.05 (m, 2H), 3.03-3.00 (m, 1H), 2.85-2.79 (m, 1H), 2.34 (s, 3H), 2.18-2.05 (m, 2H), 2.04-1.98 (m, 1H), 1.87-1.83 (m, 1H), 1.83-1.75 (m, 2H), 1.56 (d, J=6.8 Hz, 3H). LC-MS: (ESI, m/z): 694.4 [M+H]⁺

### Example 632: 3-((R)-1-(5-(6-amino-5-fluoro-4-methyl-3-(trifluoromethyl)pyridin-2-yl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyrazin-2-amine

### Synthetic Route

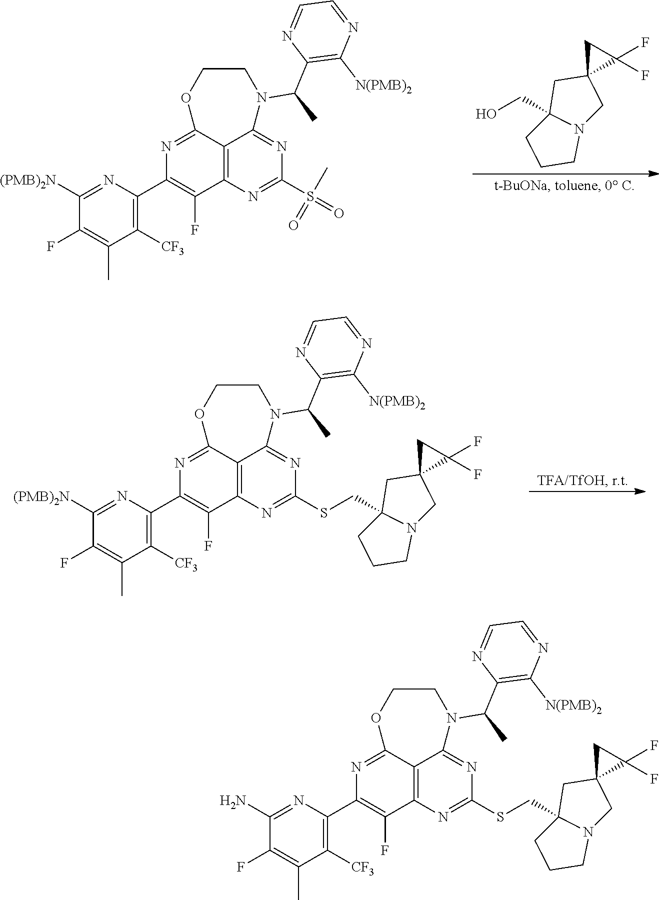

### Step 1: 3-((R)-1-(5-(6-(bis(4-methoxybenzyl)amino)-5-fluoro-4-methyl-3-(trifluoromethyl)pyridin-2-yl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine

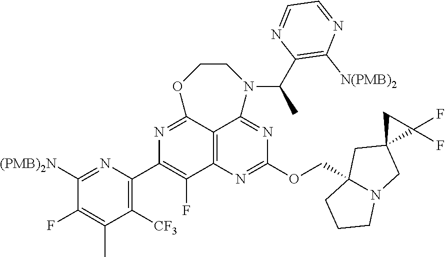

A solution of ((1R,7a'S)-2,2-difluorodihydro-1′H,3′H-spiro[cyclopropane-1,2′-pyrrolizin]-7a′(5′H)-yl)methanol (61.0 mg, 0.29 mmol) and sodium tert-butoxide (40.0 mg, 0.42 mmol) in toluene (1.6 mL) was stirred at 0° C. for 10 minutes. Then (R)-3-(1-(5-(6-(bis(4-methoxybenzyl)amino)-5-fluoro-4-methyl-3-(trifluoromethyl)pyridin-2-yl)-4-fluoro-2-(methylsulfonyl)-8,9-dihydro-10H-7-oxa-1,3,6,10-tetraazacyclohepta[de]naphthalen-10-yl)ethyl)-N,N-bis(4-methoxybenzyl)pyrazin-2-amine (160.0 mg, 0.15 mmol) was added and stirred at 0° C. for 10 minutes. After completion, the reaction mixture was diluted with water. The resulting solution was extracted with ethyl acetate and the organic layers were combined. The organic layer was dried over anhydrous sodium sulfate and concentrated under vacuum. The resulting residue was purified by reverse phase chromatography (acetonitrile/0.1% ammonium bicarbonate in water) to afford 3-((R)-1-(5-(6-(bis(4-methoxybenzyl)amino)-5-fluoro-4-methyl-3-(trifluoromethyl)pyridin-2-yl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine (90.0 mg, 0.07 mmol, 54.2% yield) as a yellow solid. LC-MS: (ESI, m/z): 1201.2 [M+H]⁺

### Step 2: 3-((R)-1-(5-(6-amino-5-fluoro-4-methyl-3-(trifluoromethyl)pyridin-2-yl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyrazin-2-amine

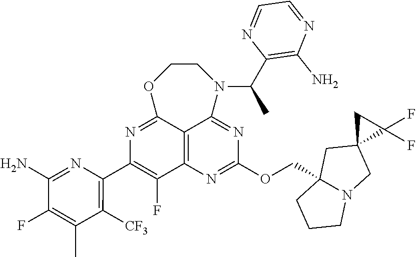 [SMILES: Cc1c(F)c(N)nc(-c2nc3c4c(nc(OC[C@@]56CCCN5C[C@]5(CC5(F)F)C6)nc4c2F)N([C@H](C)c2nccnc2N)CCO3)c1C(F)(F)F]

A solution of 3-((R)-1-(5-(6-(bis(4-methoxybenzyl)amino)-5-fluoro-4-methyl-3-(trifluoromethyl)pyridin-2-yl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)-N,N-bis(4-methoxybenzyl)pyrazin-2-amine (90.0 mg, 0.07 mmol) in trifluoroacetic acid (0.9 mL) and trifluoromethanesulfonic acid (0.09 mL) was stirred at 25° C. for 20 minutes. After completion, the reaction mixture was concentrated under vacuum, diluted with ethyl acetate, adjusted to pH>7 with sodium bicarbonate saturated solution, extracted with ethyl acetate and the organic layer was combined. The organic layer was dried over anhydrous sodium sulfate and concentrated under vacuum. The product was purified by Prep-HPLC with the following conditions: Column: XSelect CSH Fluoro Phenyl 30*150 mm, 5 μm; Mobile Phase A: Water(10 mmol/L NH₄HCO₃), Mobile Phase B: ACN; Flow rate: 60 mL/min; Gradient: 35% B to 60% B in 9 min; Wave Length: 254 nm/220 nm to afford 3-((R)-1-(5-(6-amino-5-fluoro-4-methyl-3-(trifluoromethyl)pyridin-2-yl)-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyrazin-2-amine (30.7 mg, 0.04 mmol, 56.8% yield) as a white solid. LC-MS: (ESI, m/z): 721.2 [M+H]⁺

Example 632: ¹H NMR (500 MHz, DMSO-d₆, ppm) δ 7.95 (d, J=2.6 Hz, 1H), 7.78 (d, J=2.7 Hz, 1H), 7.16 (s, 2H), 6.39-6.34 (m, 3H), 4.52 (dd, J=12.8, 6.4 Hz, 1H), 4.40 (dd, J=12.7, 6.8 Hz, 1H), 4.25 (d, J=10.5 Hz, 1H), 4.13 (d, J=10.5 Hz, 1H), 3.88 (dd, J=15.8, 6.8 Hz, 1H), 3.68 (dd, J=15.9, 6.4 Hz, 1H), 3.08 (dd, J=11.9, 6.8 Hz, 1H), 3.03-2.99 (m, 1H), 2.70 (d, J=11.9 Hz, 1H), 2.55-2.52 (m, 1H), 2.33 (s, 3H), 2.05 (dd, J=13.4, 5.7 Hz, 1H), 2.01-1.96 (m, 1H), 1.88 (d, J=13.4 Hz, 1H), 1.84-1.69 (m, 2H), 1.65-1.52 (m, 5H), 1.50-1.44 (m, 1H). LC-MS: (ESI, m/z): 721.2 [M+H]⁺

### Example 633: 6-(10-((R)-1-(2-aminopyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-4-methyl-5-(trifluoromethyl)pyridin-2-amine

### Synthetic Route

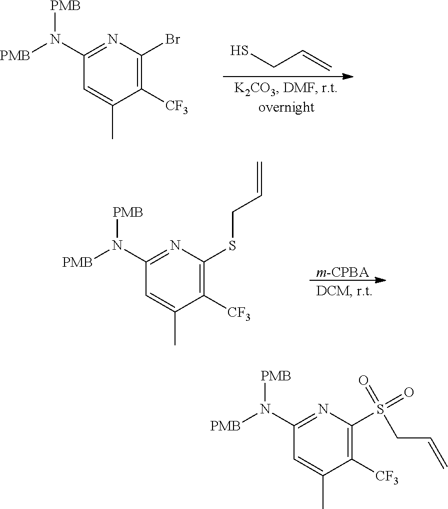

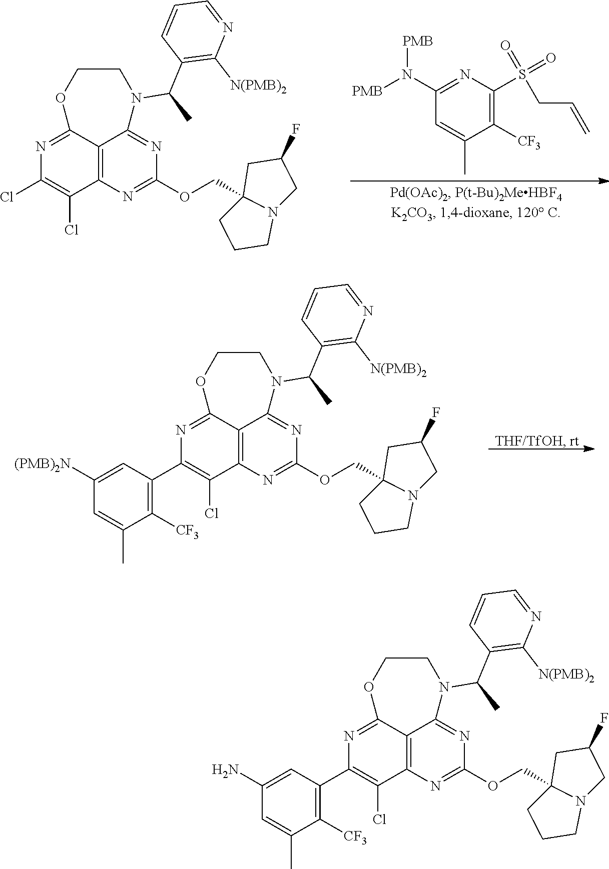

### Step 1: 6-(allylthio)-N,N-bis(4-methoxybenzyl)-4-methyl-5-(trifluoromethyl)pyridin-2-amine

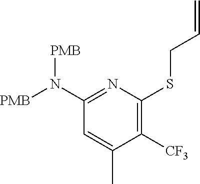

Under nitrogen, a solution of 6-bromo-N,N-bis(4-methoxybenzyl)-4-methyl-5-(trifluoromethyl)pyridin-2-amine (4.8 g, 9.69 mmol), allyl mercaptan (3.58 g, 48.29 mmol) and potassium carbonate (2.68 g, 19.39 mmol) in N,N-dimethylformamide (48 mL) was stirred at 25° C. for 16 hours. After completion, the reaction mixture was diluted with water. The resulting solution was extracted with ethyl acetate and the organic layers were combined. The organic layer was dried over anhydrous sodium sulfate and concentrated under vacuum. The residue was purified by flash chromatography on silica gel eluting with petroleum ether/ethyl acetate (10:1) to afford 6-(allylthio)-N,N-bis(4-methoxybenzyl)-4-methyl-5-(trifluoromethyl)pyridin-2-amine (4.36 g, 8.9 mmol, 92.1% yield) as a colorless oil. LC-MS: (ESI, m/z): 489.1 [M+H]⁺

### Step 2: 6-(allylsulfonyl)-N,N-bis(4-methoxybenzyl)-4-methyl-5-(trifluoromethyl)pyridin-2-amine

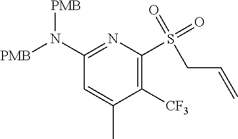

A solution of 6-(allylthio)-N,N-bis(4-methoxybenzyl)-4-methyl-5-(trifluoromethyl)pyridin-2-amine (4.30 g, 8.80 mmol) and 3-chloroperoxybenzoic acid (4.58 g, 26.54 mmol) in dichloromethane (45 mL) was stirred at 25° C. for 4 hours. After completion, the resulting solution was quenched with saturated sodium bisulfite solution and extracted with ethyl acetate. Then the organic layers were combined, washed with brine, dried over anhydrous sodium sulfate and concentrated under vacuum. The residue was purified by flash chromatography on silica gel eluting with petroleum ether/ethyl acetate (7/3) to afford 6-(allylsulfonyl)-N,N-bis(4-methoxybenzyl)-4-methyl-5-(trifluoromethyl)pyridin-2-amine (3.06 g, 5.88 mmol, 66.8% yield) as a yellow solid. LC-MS: (ESI, m/z): 521.2 [M+H]⁺

### Step 3: 6-(10-((R)-1-(2-(bis(4-methoxybenzyl)amino)pyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-4-methyl-5-(trifluoromethyl)pyridin-2-amine

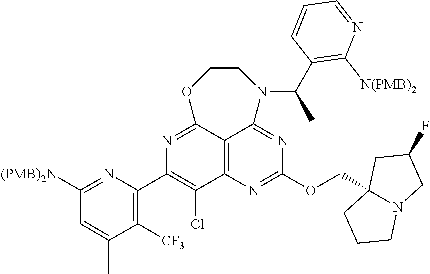

Under nitrogen, a mixture of 6-(allylsulfonyl)-N,N-bis(4-methoxybenzyl)-4-methyl-5-(trifluoromethyl)pyridin-2-amine (235.0 mg, 0.45 mmol), 3-((R)-1-(4,5-dichloro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)-N,N-bis(4-methoxybenzyl)pyridin-2-amine (174.0 mg, 0.22 mmol), palladium (II) acetate (11.0 mg, 0.0700 mmol), di-tert-b-butylmethylphosphonium tetrafluoroborate (33.0 mg, 0.13 mmol) and potassium carbonate (62.0 mg, 0.45 mmol) in 1,4-dioxane (1.7 mL) stirred at 120° C. for 5 hours. After completion, the resulting solution was diluted with water and extracted with ethyl acetate. The organic layer was washed with brine and dried over anhydrous sodium sulfate and concentrated under vacuum. The resulting residue was purified by reverse phase chromatography (acetonitrile/0.1% ammonium bicarbonate in water) to afford 6-(10-((R)-1-(2-(bis(4-methoxybenzyl)amino)pyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-4-methyl-5-(trifluoromethyl)pyridin-2-amine (60.0 mg, 0.05 mmol, 23.1% yield) as a yellow solid. LC-MS: (ESI, m/z): 1154.6 [M+H]⁺

### Step 4: 6-(10-((R)-1-(2-aminopyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-4-methyl-5-(trifluoromethyl)pyridin-2-amine

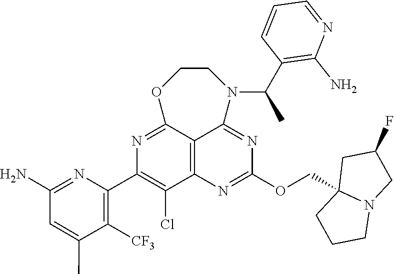 [SMILES: Cc1cc(N)nc(-c2nc3c4c(nc(OC[C@@]56CCCN5C[C@H](F)C6)nc4c2Cl)N([C@H](C)c2cccnc2N)CCO3)c1C(F)(F)F]

A solution of 6-(10-((R)-1-(2-(bis(4-methoxybenzyl)amino)pyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-N,N-bis(4-methoxybenzyl)-4-methyl-5-(trifluoromethyl)pyridin-2-amine (57.0 mg, 0.05 mmol) in trifluoroacetic acid (1 mL) and trifluoromethanesulfonic acid (0.1 mL) was stirred at 25° C. for 0.5 hours. After completion, the mixture was concentrated under vacuum. The mixture was diluted with dichloromethane. The residue was adjusted to pH=7 by saturated sodium bicarbonate solution and washed with water. The organic layer was washed with brine and dried over anhydrous sodium sulfate and concentrated under vacuum. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 22% B to 42% B in 9 min; Wave Length: 254 nm/220 nm; RT1(min): 8.7 to afford 6-(10-((R)-1-(2-aminopyridin-3-yl)ethyl)-4-chloro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-4-methyl-5-(trifluoromethyl)pyridin-2-amine (7.8 mg, 0.01 mmol, 23.4% yield) as a white solid. LC-MS: (ESI, m/z): 674.2 [M+H]⁺

Example 633: ¹H NMR (300 MHz, DMSO-d₆, ppm) δ 8.01-7.93 (m, 1H), 7.63 (d, J=7.4 Hz, 1H), 6.75 (s, 2H), 6.71-6.63 (m, 1H), 6.46 (s, 1H), 6.33 (q, J=9.6, 8.9 Hz, 1H), 5.70 (s, 2H), 5.29 (d, J=54.4 Hz, 1H), 4.54-4.23 (m, 2H), 4.22-4.07 (m, 2H), 3.84-3.60 (m, 1H), 3.44-3.37 (m, 1H) 3.18-2.95 (m, 3H), 2.91-2.78 (m, 1H), 2.35 (s, 3H), 2.20-2.00 (m, 3H), 1.90-1.69 (m, 3H), 1.56 (t, J=6.5 Hz, 3H). LC-MS: (ESI, m/z): 674.2 [M+H]⁺

### Example 634: 3-((1R)-1-(4-fluoro-5-(6-fluoro-1-methyl-1H-indazol-7-yl)-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

### Synthetic Route

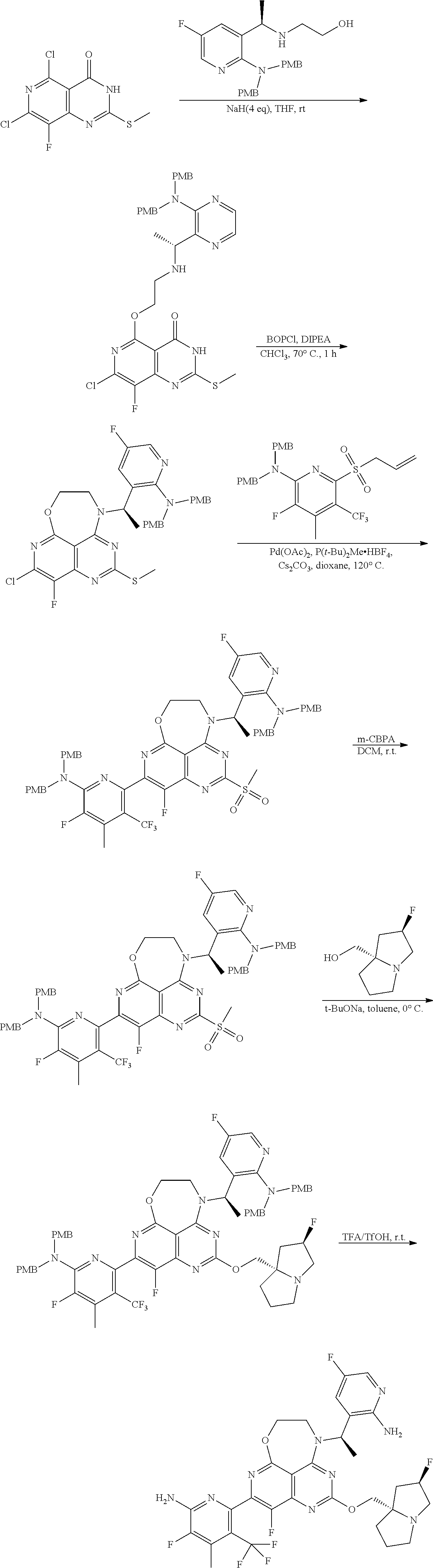

### Step 1: (R)-5-(2-((1-(2-aminopyridin-3-yl)ethyl)amino)ethoxy)-7-chloro-8-fluoro-2-(methylthio)pyrido[4,3-d]pyrimidin-4(3H)-one

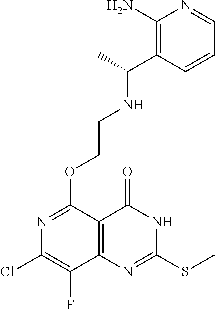 [SMILES: CSc1nc2c(F)c(Cl)nc(OCCN[C@H](C)c3cccnc3N)c2c(=O)[nH]1]

To a mixture of 2-[[(1R)-1-(2-amino-3-pyridyl)ethyl]amino]ethanol (750.0 mg, 4.14 mmol) in tetrahydrofuran (20 mL) was added sodium hydride (700.0 mg, 17.5 mmol, 60% dispersion in mineral oil), the mixture was stirred for 10 minutes at 0° C. Then 5,7-dichloro-8-fluoro-2-methylsulfanyl-3H-pyrido[4,3-d]pyrimidin-4-one (1000.0 mg, 3.57 mmol) was added, stirred for 1 h at room temperature. After completion, the reaction was quenched with saturated ammonium chloride solution. The resulting solution was diluted with water and extracted with ethyl acetate. The organic layers were combined, washed with brine, dried over anhydrous sodium sulfate and concentrated. The crude product would be directly used in the next step without purification. LCMS (ESI, m/z): 425.1 [M+H]⁺.

### Step 2: (R)-3-(1-(5-chloro-4-fluoro-2-(methylthio)-8,9-dihydro-10H-7-oxa-1,3,6,10-tetraazacyclohepta[de]naphthalen-10-yl)ethyl)pyridin-2-amine

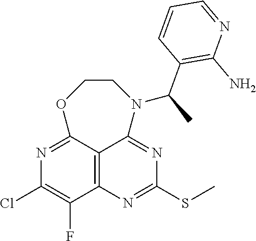 [SMILES: CSc1nc2c3c(nc(Cl)c(F)c3n1)OCCN2[C@H](C)c1cccnc1N]

A mixture of (R)-5-(2-((1-(2-aminopyridin-3-yl)ethyl)amino)ethoxy)-7-chloro-8-fluoro-2-(methylthio)pyrido[4,3-d]pyrimidin-4(3H)-one (2.00 g, crude), bis(2-oxo-3-oxazolidinyl)phosphinicchloride (1.50 g, 5.89 mmol) and N,N-diisopropylethylamine (4.04 mL, 23.21 mmol) in chloroform (40 mL) was stirred at 70° C. for 1 hour. The resulting solution was diluted with water and extracted with ethyl acetate. The organic layers were combined, washed with brine, dried over anhydrous sodium sulfate and concentrated. The crude product would be directly used in the next step without purification. LCMS (ESI, m/z): 407.1 [M+H]⁺.

### Step 3: tert-butyl N-tert-butoxycarbonyl-N-[3-[(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl]-2-pyridyl]carbamate

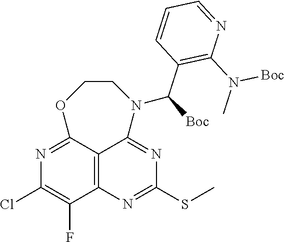 [SMILES: CSc1nc2c3c(nc(Cl)c(F)c3n1)OCCN2[C@H](C(=O)OC(C)(C)C)c1cccnc1N(C)C(=O)OC(C)(C)C]

A solution of (R)-3-(1-(5-chloro-4-fluoro-2-(methylthio)-8,9-dihydro-10H-7-oxa-1,3,6,10-tetraazacyclohepta[de]naphthalen-10-yl)ethyl)pyridin-2-amine (2.00 g, crude), di-tert-butyl dicarbonate (3.20 g, 14.66 mmol), triethylamine (6.89 mL, 49.41 mmol) and 4-dimethylaminopyridine (120.0 mg, 0.98 mmol) in tetrahydrofuran (40 mL) was stirred at 60° C. for 2 hours. After completion, the reaction was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (3:7) to afford tert-butyl N-tert-butoxycarbonyl-N-[3-[(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl]-2-pyridyl]carbamate (860 mg, 1.42 mmol, 28.8% yield) as a yellow solid. LCMS (ESI, m/z): 607.2 [M+H]⁺.

### Step 4: tert-butyl N-tert-butoxycarbonyl-N-[3-[(1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl]-2-pyridyl]carbamate

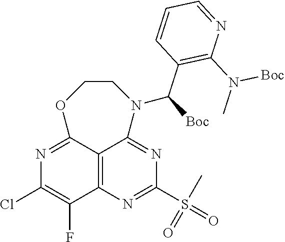 [SMILES: CN(C(=O)OC(C)(C)C)c1ncccc1[C@@H](C(=O)OC(C)(C)C)N1CCOc2nc(Cl)c(F)c3nc(S(C)(=O)=O)nc1c23]

A mixture of tert-butyl N-tert-butoxycarbonyl-N-[3-[(1R)-1-(7-chloro-6-fluoro-3-methylsulfanyl-10-oxa-2,4,8,13-tetrazatricyclo[7.4.1.05, 14]tetradeca-1,3,5(14),6,8-pentaen-13-yl)ethyl]-2-pyridyl]carbamate (400.0 mg, 0.66 mmol) and 3-chloroperoxybenzoicacid (340.0 mg, 1.97 mmol) in dichloromethane (10 mL) was stirred at room temperature for 1 hour. The reaction was quenched with saturated sodium sulfite solution. The resulting solution was extracted with ethyl acetate. The organic layers were combined, washed with brine, dried over anhydrous sodium sulfate and concentrated. The residue was purified by flash chromatography on silica gel eluting with petroleum ether/ethyl acetate (30:70) to afford tert-butyl N-tert-butoxycarbonyl-N-[3-[(1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl]-2-pyridyl]carbamate (240 mg, 0.38 mmol, 57% yield) as a yellow solid. LCMS (ESI, m/z):639.2 [M+H]⁺.

### Step 5: tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate

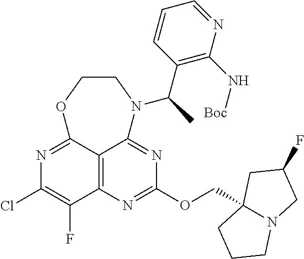 [SMILES: C[C@H](c1cccnc1NC(=O)OC(C)(C)C)N1CCOc2nc(Cl)c(F)c3nc(OC[C@@]45CCCN4C[C@H](F)C5)nc1c23]

Under nitrogen, to a mixture of ((2R,7aS)-2-fluorotetrahydro-1H-pyrrolizin-7a(5H)-yl)methanol (145.0 mg, 0.91 mmol) in toluene (7 mL) was added sodium tert-butoxide (130.8 mg, 1.36 mmol), the mixture was stirred for 10 minutes at 0° C., then tert-butyl N-tert-butoxycarbonyl-N-[3-[(1R)-1-(7-chloro-6-fluoro-3-methylsulfonyl-10-oxa-2,4,8,13-tetrazatricyclo[7.4.1.05, 14]tetradeca-1,3,5(14),6,8-pentaen-13-yl)ethyl]-2-pyridyl]carbamate (290.0 mg, 0.45 mmol) was added, stirred for 1 hour at room temperature. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (96:4) to afford tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (180 mg, 0.29 mmol, 64.2% yield) as a yellow solid. LCMS (ESI, m/z):618.2 [M+H]⁺.

### Step 6: tert-butyl (3-((1R)-1-(4-fluoro-5-(6-fluoro-1-methyl-1H-indazol-7-yl)-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate

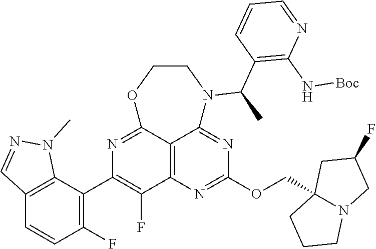 [SMILES: C[C@H](c1cccnc1NC(=O)OC(C)(C)C)N1CCOc2nc(-c3c(F)ccc4cnn(C)c34)c(F)c3nc(OC[C@@]45CCCN4C[C@H](F)C5)nc1c23]

Under nitrogen, a solution of tert-butyl (3-((R)-1-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (200.0 mg, 0.28 mmol), (6-fluoro-1-methyl-1H-indazol-7-yl)boronic acid (81.0 mg, 0.42 mmol), tetrakis(triphenylphosphine)palladium (32.0 mg, 0.03 mmol) and potassium carbonate (77.0 mg, 0.56 mmol) in 1,4-dioxane (2 mL) and water (0.4 mL) was stirred for 1 hour at 100° C. After completion, the resulting solution was diluted with ethyl acetate and washed with water. The organic layer was washed with brine and dried over anhydrous sodium sulfate and concentrated under vacuum. The residue was purified by flash chromatography on silica gel eluting with dichloromethane/methanol (9:1) to afford tert-butyl (3-((1R)-1-(4-fluoro-5-(6-fluoro-1-methyl-1H-indazol-7-yl)-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (164 mg, 0.1971 mmol, 70.8% yield) as a brown solid. LC-MS: (ESI, m/z): 732.4 [M+H]⁺

### Step 7: 3-((1R)-1-(4-fluoro-5-(6-fluoro-1-methyl-1H-indazol-7-yl)-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine

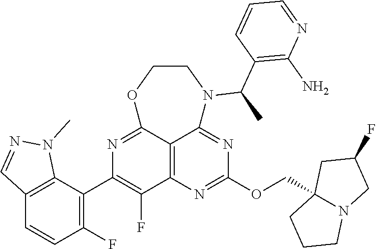 [SMILES: C[C@H](c1cccnc1N)N1CCOc2nc(-c3c(F)ccc4cnn(C)c34)c(F)c3nc(OC[C@@]45CCCN4C[C@H](F)C5)nc1c23]

A solution of tert-butyl (3-((1R)-1-(4-fluoro-5-(6-fluoro-1-methyl-1H-indazol-7-yl)-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-yl)carbamate (150.0 mg, 0.18 mmol) in dichloromethane (3 mL) and trifluoroacetic acid (2 mL) was stirred at 25° C. for 0.5 hours. After completion, the mixture was concentrated under vacuum. The residue was adjusted to pH=7 by saturated sodium bicarbonate solution. The mixture was diluted with dichloromethane and washed with water. The organic layer was washed with brine and dried over anhydrous sodium sulfate and concentrated under vacuum. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 35% B to 54% B in 9 min; Wave Length: 254 nm/220 nm; RT1(min): 8.25 to afford 3-((1R)-1-(4-fluoro-5-(6-fluoro-1-methyl-1H-indazol-7-yl)-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethyl)pyridin-2-amine (60.0 mg, 0.10 mmol, 52.7% yield) as a white solid. LC-MS: (ESI, m/z): 632.6 [M+H]⁺

Example 634: ¹H NMR (300 MHz, DMSO-d₆, ppm) δ 8.29 (s, 1H), 7.97 (dd, J=4.9, 1.7 Hz, 1H), 7.63 (dd, J=7.5, 1.8 Hz, 1H), 7.53-7.41 (m, 1H), 7.06 (dd, J=9.8, 8.0 Hz, 1H), 6.67 (dd, J=7.5, 4.9 Hz, 1H), 6.36 (q, J=6.8 Hz, 1H), 5.73 (s, 2H), 5.28 (d, J=53.8 Hz, 1H), 4.45 (dd, J=12.7, 6.5 Hz, 1H), 4.31 (dd, J=12.6, 6.3 Hz, 1H), 4.15 (s, 2H), 3.78-3.70 (m, 4H), 3.41 (dd, J=15.9, 6.5 Hz, 1H), 3.16-2.98 (m, 3H), 2.89-2.75 (m, 1H), 2.24-2.12 (m, 1H), 2.09-1.97 (m, 2H), 1.90-1.72 (m, 3H), 1.57 (d, J=6.8 Hz, 3H). LC-MS: (ESI, m/z): 632.6 [M+H]⁺

### Example 635: 6-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-4-methyl-5-(trifluoromethyl)pyridin-2-amine

### Synthetic Route

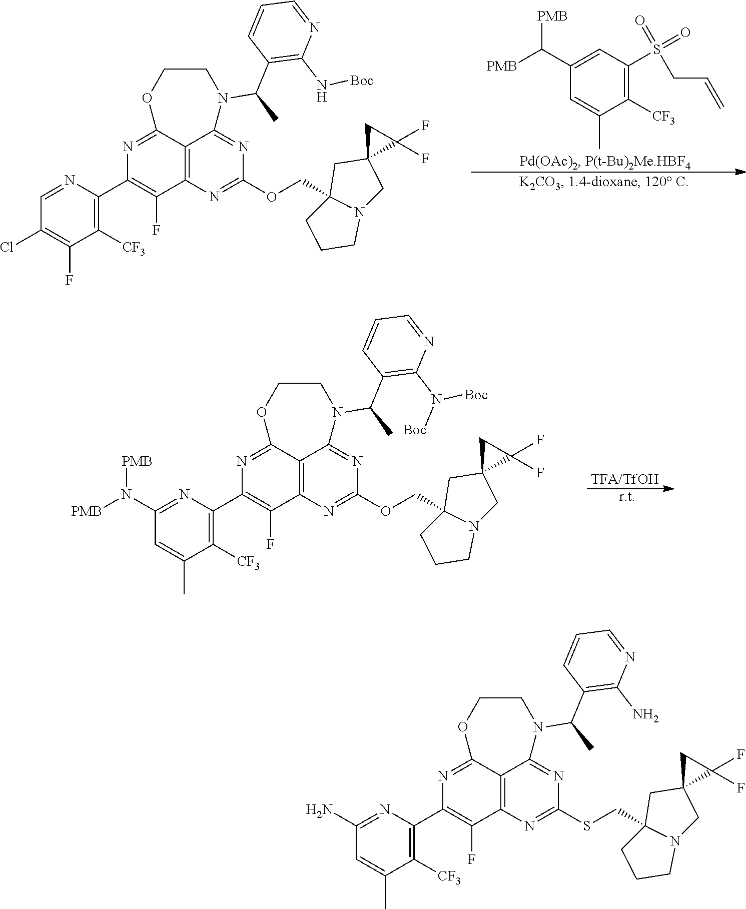

### Step 1: 6-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-N,N-bis(4-methoxybenzyl)-4-methyl-5-(trifluoromethyl)pyridin-2-amine

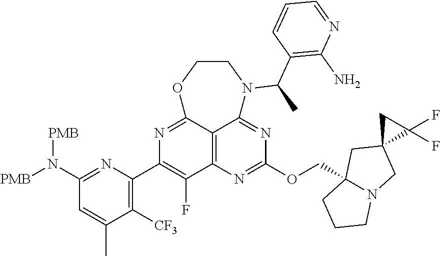

Under nitrogen, a solution of tert-butyl (3-((R)-1-(5-chloro-2-(((1R,7a'S)-2,2-difluorodihydro-1′H,3′H-spiro[cyclopropane-1,2′-pyrrolizin]-7a′(5′H)-yl)methoxy)-4-fluoro-8,9-dihydro-10H-cyclohepta[de]naphthalen-10-yl)ethyl)pyridin-2-yl)carbamate (305.0 mg, 0.40 mmol), 6-(allylsulfonyl)-N,N-bis(4-methoxybenzyl)-4-methyl-5-(trifluoromethyl)pyridin-2-amine (334.0 mg, 0.64 mmol), di-tert-b-butylmethylphosphonium tetrafluoroborate (34.0 mg, 0.14 mmol), palladium (II) acetate (11.0 mg, 0.07 mmol) and potassium carbonate (117.0 mg, 0.85 mmol) in 1,4-dioxane (3 mL) was stirred for 16 hours at 120° C. After completion, the resulting solution was diluted with ethyl acetate and washed with water. The organic layer was washed with brine and dried over anhydrous sodium sulfate and concentrated under vacuum. The resulting residue was purified by reverse phase chromatography (acetonitrile/0.1% ammonium bicarbonate in water) to afford 6-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-N,N-bis(4-methoxybenzyl)-4-methyl-5-(trifluoromethyl)pyridin-2-amine (137.0 mg, 0.12 mmol, 30.0% yield) as an orange solid. LC-MS: (ESI, m/z): 942.4 [M+H]⁺

### Step 2: 6-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-4-methyl-5-(trifluoromethyl)pyridin-2-amine

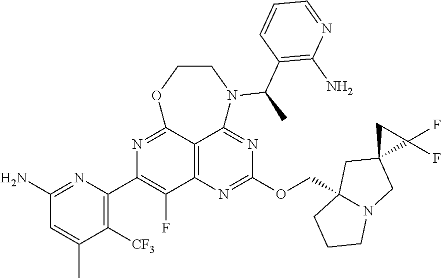 [SMILES: Cc1cc(N)nc(-c2nc3c4c(nc(OC[C@@]56CCCN5C[C@]5(CC5(F)F)C6)nc4c2F)N([C@H](C)c2cccnc2N)CCO3)c1C(F)(F)F]

A solution of 6-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-N,N-bis(4-methoxybenzyl)-4-methyl-5-(trifluoromethyl)pyridin-2-amine (123.0 mg, 0.11 mmol) in trifluoroacetic acid (3 mL) and trifluoromethanesulfonic acid (0.3 mL) was stirred at 25° C. for 0.5 hours. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 40% B to 55% B in 10 min; Wave Length: 254 nm/220 nm; RT1(min): 8.5 to afford 6-(10-((R)-1-(2-aminopyridin-3-yl)ethyl)-2-(((1R,7a'S)-2,2-difluorodihydro-1′H,3′H-spiro[cyclopropane-1,2′-pyrrolizin]-7a′(5′H)-yl)methoxy)-4-fluoro-9,10-dihydro-8H-cyclohepta[de]naphthalen-5-yl)-4-methyl-5-(trifluoromethyl)pyridin-2-amine (28.6 mg, 0.04 mmol, 37.9% yield) as a white solid. LC-MS: (ESI, m/z): 702.2 [M+H]⁺

Example 635: ¹H NMR (300 MHz, DMSO-d₆, ppm) δ 7.97 (dd, J=4.9, 2.3 Hz, 1H), 7.63 (d, J=7.6 Hz, 1H), 6.77 (s, 2H), 6.71-6.62 (m, 1H), 6.48 (s, 1H), 6.35 (q, J=6.8 Hz, 1H), 5.70 (s, 2H), 4.42 (dd, J=12.9, 6.4 Hz, 1H), 4.35-4.14 (m, 3H), 3.71 (dd, J=15.9, 6.4 Hz, 1H), 3.42-3.38 (m, 1H), 3.10 (dd, J=12.1, 6.7 Hz, 1H), 3.05-2.96 (m, 1H), 2.72 (d, J=11.8 Hz, 1H), 2.60-2.53 (m, 1H), 2.35 (s, 3H), 2.13 (dd, J=13.7, 5.8 Hz, 1H), 2.04-1.96 (m, 1H), 1.95-1.86 (m, 1H), 1.85-1.71 (m, 2H), 1.68-1.41 (m, 6H) LC-MS: (ESI, m/z): 702.2 [M+H]⁺

### Further Examples: Biological Assays

### KRAS Biochemical Assay—BODIPY-GDP Exchange TR-FRET.

Biochemical compound potencies were assessed by evaluating inhibition of SOS1-mediated nucleotide exchange in KRAS G12D. The SOS1-promoted exchange of fluorescently-labeled GDP (BOPIDY-GDP) was monitored by time-resolved fluorescence resonance energy transfer (TR-FRET). Compounds solubilized in DMSO were dispensed as concentration series into 384-well white assay plates. A preformed complex of biotin-tagged recombinant human KRAS (1.5 nM mutant KRas or wild type) and 0.15 nM terbium-labeled streptavidin (CisBIO) prepared in 10 μL/well assay buffer (20 mM HEPES, pH 7.5, 50 mM NaCl, 10 mM MgCl₂, 0.01% Tween-20 and 1 mM dithiothreitol) was added and allowed to incubate for 10-minutes. The reaction was initiated with the addition of 5 μL of 3 nM recombinant human SOS1 and 300 nM BODIPY-GDP in assay buffer. After a 60-minute incubation, the fluorescence was measured with excitation at 337 nm and emission at 490 and 520 nm. The TR-FRET ratio was determined as the fluorescence at 520 nm divided by the fluorescence at 490 nm multiplied by 10,000. The results were normalized to percent inhibition based on control samples: DMSO (0% inhibition) and control compound at a concentration that inhibits completely (100% inhibition). The normalized TR-FRET results were plotted against compound concentration, and the data fit to a 4-parameter Hill equation to determine the IC₅₀ values.

SW620 and HPAC 3D proliferation assays.

Compound potencies were assessed in proliferation assays conducted with SW620 and HPAC cell lines grown in 3D cultures. Cells were seeded into 96- or 384-well ultra-low attachment plates at 3000 or 1000 cells/well, respectively, in culture medium (RPMI-1640, 10% FBS, 2 mM L-glutamine). The plated cells were centrifuged briefly, allowed to rest for 30 minutes under ambient conditions, and cultured overnight at 37° C./5% CO₂ to initiate the formation of spheroids. Compound dilutions were prepared across a range of concentrations in DMSO and added to the appropriate wells of the assay plates with a 333-fold dilution into the culture medium. The plates were further incubated for 7 days. The quantity of viable cells was assessed using CellTiter-Glo 3D Cell Viability Assay (Promega). The detection reagent penetrates the spheroids and lyses the cells. The released ATP that was detected with luciferase and a luciferase substrate to generate a luminescent readout. The luminescence values were normalized to controls (DMSO alone for 0% inhibition and a maximally inhibitory concentration of a control compound for 100% inhibition) and plotted against compound concentration. The data points were fit to a 4-parameter logistic equation to determine IC50 values.

TABLE 3
Activities of compounds (reported in μM)

| Ex. No. | WT-GDP HTRF IC50 | G12V-GDP HTRF IC50 |
|---|---|---|
| 1 | 0.000114 | 0.0001 |
| 2 | 0.0000671 | 0.00008 |
| 3 | 0.000104 | 0.00008 |
| 4 | 0.000133 | 0.00011 |
| 5 | 0.0000529 | 0.000045 |
| 6 | 0.000215 | 0.00021 |
| 7 | 0.00012 | 0.00004 |
| 8 | 0.0539 | 0.0028 |
| 9 | 0.000526 | 0.0001 |
| 10 | 0.00595 | 0.00047 |
| 11 | 0.00007 | 0.00006 |
| 12 | 0.000175 | 0.00006 |
| 13 | 0.0018 | 0.00029 |
| 14 | 0.000434 | 0.00017 |
| 15 | 0.00052 | 0.00017 |
| 16 | 0.00018 | 0.00006 |
| 17 | 0.000159 | 0.00008 |
| 18 | 0.00033 | 0.00024 |
| 19 | 0.00693 | 0.00085 |
| 20 | 0.00372 | 0.0003 |
| 21a | 0.00293 | 0.0019 |
| 21b | 0.00007 | 0.00005 |
| 22 | 0.000825 | 0.00030 |
| 23 | 0.00086 | 0.00016 |
| 24 | 0.00172 | 0.00023 |
| 25 | 0.00037 | 0.00012 |
| 26 | 0.00026 | 0.00011 |
| 27 | 0.00013 | 0.00004 |
| 28a | 0.00316 | 0.00037 |
| 28b | 0.0015 | 0.0003 |
| 28c | 0.0811 | 0.021 |
| 28d | 1.57 | 0.42 |
| 29 | 0.00015 | 0.00009 |
| 30 | 0.000114 | 0.00006 |
| 31 | 0.000419 | 0.00027 |
| 32 | 0.000223 | 0.00013 |
| 633 | 0.000416 | 2.0E−4 |
| 604 | 0.000225 | 9.9E−5 |
| 626 | 0.00067 | 1.6E−4 |
| 634 | 0.0373 | 0.0029 |
| 627 | 0.00467 | 4.4E−4 |
| 616 | 0.000195 | 9.0E−5 |
| 635 | 0.000325 | 2.1E−4 |
| 617 | 0.00031 | 1.1E−4 |
| 602 | 0.0226 | 0.0022 |
| 618 | 0.00343 | 4.5E−4 |
| 608 | 0.00338 | 5.2E−4 |
| 620 | 0.000748 | 2.4E−4 |
| 605 | 0.00019 | 1.4E−4 |
| 606 | 0.000808 | 2.0E−4 |
| 621 | 0.00228 | 1.7E−4 |
| 609 | 0.000415 | 2.1E−4 |
| 623 | 0.00011 | 5.0E−5 |
| 629a | 0.000178 | 6.9E−5 |
| 629b | 0.0123 | 0.0048 |
| 612 | 0.00178 | 2.3E−4 |
| 622a | 0.000572 | 1.9E−4 |
| 622b | 0.0055 | 0.0023 |
| 601a | 0.000255 | 7.0E−5 |
| 601b | 0.00316 | 7.6E−4 |
| 625 | 0.0012 | 1.2E−4 |
| 630 | 0.002 | 3.2E−4 |
| 614 | 0.00016 | 1.2E−4 |
| 615 | 0.000175 | 1.4E−4 |
| 624 | 0.000722 | 9.0E−5 |
| 628 | 0.000826 | 3.3E−4 |
| 613 | 0.00145 | 2.4E−4 |
| 631 | 0.000741 | 2.1E−4 |
| 632 | 0.000379 | 8.9E−5 |
| 607 | 0.00069 | 2.7E−4 |

TABLE 4
Activities of compounds (reported in μM)

| | SW620 | H1975 | HPAC | SW480 | SW620 |
|---|---|---|---|---|---|
| Ex. | CTG | CTG | CTG | CTG | PERK |
| No. | IC50 | IC50 | IC50 | IC50 | IC50 |
| 1 | 0.007 | 0.0474 | 0.144 | 0.026 | 0.00142 |
| 2 | 0.00241 | 0.00259 | 0.0208 | 0.0029 | 0.00129 |
| 3 | 0.00115 | 0.00954 | 0.00879 | ND | 0.00059 |
| 4 | 0.00126 | 0.00259 | 0.00572 | 0.0024 | 0.00107 |
| 5 | 0.00114 | 0.0128 | 0.00506 | 0.0058 | 0.00072 |
| 6 | 0.0134 | 0.00723 | 0.0437 | 0.12 | 0.00768 |
| 7 | 0.00196 | 0.00259 | 0.0193 | 0.012 | 0.00113 |
| 8 | 0.456 | 0.555 | 2 | ND | 0.139 |
| 9 | 0.0262 | 0.0112 | 0.0747 | ND | 0.0193 |
| 10 | 0.114 | 0.0534 | 0.439 | ND | 0.0299 |
| 11 | 0.00532 | 0.00259 | 0.173 | ND | 0.0019 |
| 12 | 0.00563 | 0.0594 | 0.0147 | 0.0028 | 0.00162 |
| 13 | 0.0267 | 0.0734 | 0.29 | ND | ND |
| 14 | 0.0139 | 0.116 | 0.076 | 0.13 | 0.00265 |
| 15 | 0.00074 | 0.00259 | 0.00884 | 0.01 | 0.00224 |
| 16 | 0.00377 | 0.0311 | 0.0302 | 0.01 | 0.00253 |
| 17 | 0.0137 | 0.00915 | 0.228 | 0.11 | 0.00433 |
| 18 | 0.013 | 0.00554 | 0.0561 | ND | 0.00544 |
| 19 | ND | ND | ND | ND | 0.112 |
| 20 | 0.0303 | 0.421 | 0.484 | ND | 0.00859 |
| 21a | 0.00687 | 0.18 | 0.103 | ND | 0.0331 |
| 21b | 0.001 | 0.00259 | 0.00443 | 0.0024 | 0.000555 |
| 22 | 0.00645 | 0.0487 | 0.067 | 0.024 | 0.00632 |
| 23 | 0.0316 | 0.0325 | 0.092 | ND | 0.00733 |
| 24 | 1.02 | 5.17 | 2 | 0.7 | 0.466 |
| 25 | 0.00881 | 0.0831 | 0.25 | 0.11 | 0.0276 |
| 26 | 0.0323 | 0.077 | 0.416 | 0.35 | 0.0193 |
| 27 | 0.00168 | 0.00259 | 0.0552 | 0.031 | ND |
| 28a | 0.0877 | 0.0251 | 0.219 | ND | 0.032 |
| 28b | 0.0306 | 0.0246 | 0.132 | ND | 0.0365 |
| 28c | 1.92 | 4.73 | 2 | ND | 0.715 |
| 28d | 2 | 20 | 2 | ND | 2 |
| 29 | 0.00164 | 0.00259 | 0.00451 | ND | 0.00066 |
| 30 | 0.00267 | 0.00259 | 0.0158 | ND | 0.00265 |
| 31 | 0.0162 | 0.0179 | 0.156 | ND | 0.0268 |
| 32 | 0.00225 | 0.00712 | 0.0187 | ND | 0.00245 |
| 633 | 0.0199 | 0.0484 | 0.0778 | | 0.023 |
| 604 | 0.00533 | 0.283 | 0.0265 | >0.2 | 0.00293 |
| 626 | 0.0271 | 0.262 | 0.16 | | 0.0105 |
| 634 | 0.612 | 3.66 | 1.75 | | 0.0852 |
| 627 | 0.193 | 0.854 | 1.36 | | 0.0526 |
| 616 | 0.00846 | 0.00259 | 0.0504 | >0.2 | 0.00464 |
| 635 | 0.00355 | 0.00254 | 0.0118 | >0.2 | 0.00215 |
| 617 | 0.038 | 0.0244 | 0.239 | | 0.0288 |
| 602 | 1.95 | 0.296 | >2 | | 0.629 |
| 618 | 0.393 | 0.0932 | 1.05 | | 0.233 |
| 608 | 0.246 | 0.243 | 0.917 | | 0.0451 |
| 620 | 0.0192 | 0.132 | 0.0898 | | 0.00429 |
| 605 | 0.0113 | 0.004 | 0.094 | | 0.0119 |
| 606 | 1.52 | 0.374 | >2 | | 0.855 |
| 621 | 0.0306 | 0.0977 | 0.339 | | 0.0221 |
| 609 | 0.00572 | 14.5 | 0.034 | 0.058 | 0.00562 |
| 623 | 0.0033 | 4.3 | 0.0141 | 0.05 | 0.00229 |
| 629a | 0.00171 | 0.176 | 0.0153 | 0.02 | 0.00133 |
| 629b | 0.0742 | 0.426 | 0.682 | | 0.105 |
| 612 | 0.0284 | 2.5 | 0.359 | | 0.0156 |
| 622a | 0.0051 | 0.766 | 0.0396 | | 0.0032 |
| 622b | 0.0228 | 2.61 | 0.123 | | 0.0296 |
| 601a | 0.00614 | 0.0476 | 0.0336 | | 0.00234 |
| 601b | 0.0093 | 0.0802 | 0.0867 | | 0.0156 |
| 625 | 0.0624 | 0.759 | 0.451 | | 0.0593 |
| 630 | 0.0288 | 0.134 | 0.208 | | 0.0212 |
| 614 | 0.00175 | 1.96 | 0.0115 | | 0.00242 |
| 615 | 0.00192 | 0.951 | 0.0123 | 0.018 | 0.00202 |
| 624 | 0.0115 | 0.363 | 0.0705 | | 0.00556 |
| 628 | 0.0146 | 0.471 | 0.331 | | 0.014 |
| 613 | 0.29 | 0.16 | 1.52 | | 0.146 |
| 631 | 0.0473 | 0.113 | 0.11 | | 0.0165 |
| 632 | 0.0155 | 0.0834 | 0.0834 | | 0.00608 |
| 607 | 0.0907 | 0.0355 | 0.302 | | 0.0178 |

ND = Not determined

All technical and scientific terms used herein have the same meaning. Efforts have been made to ensure accuracy with respect to numbers used (e.g. amounts, temperature, etc.) but some experimental errors and deviations should be accounted for.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed herein. The upper and lower limits of these small ranges which can independently be included in the smaller rangers is also encompassed herein, subject to any specifically excluded limit in the stated range. Where the stated range includes one or both of the limits, ranges excluding either or both of those included limits are also included herein.

Many modifications and other embodiments of the compounds and methods set forth herein will come to mind to one skilled in the art having the benefit of the teachings presented in the foregoing descriptions and the associated drawings. Therefore, it is to be understood that the compounds and methods described herein are not to be limited to the specific embodiments disclosed and that modifications and other embodiments are intended to be included within the scope of the appended claims. The scope of the invention should, therefore, be determined not with reference to the above description, but should instead be determined with reference to the following appended claims, along with the full scope of equivalents to which such claims are entitled. Although specific terms are employed herein, they are used in a generic and descriptive sense only and not for purposes of limitation.

The patents, published applications, and scientific literature referred to herein establish the knowledge of those skilled in the art and are hereby incorporated by reference in their entirety to the same extent as if each was specifically and individually.

1. A compound or an atropisomer, stereoisomer, tautomer, or pharmaceutically acceptable salt thereof, having formula (I), 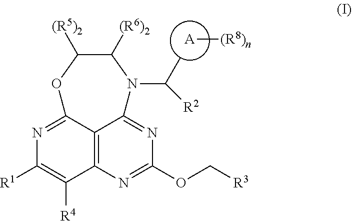
wherein:
Ring A is a R⁸-substituted or unsubstituted 3-10 membered heterocycle, or a R⁸-substituted or unsubstituted 5-10 membered heteroaryl;
R⁸ is independently halogen, CN, NH₂, NHC₁₋₃alkyl, R^{8A}-substituted or unsubstituted C₁₋₆alkyl, or R^{8A}-substituted or unsubstituted C₁₋₆haloalkyl;
R^{8A} is halogen, unsubstituted C₁₋₃alkyl, or unsubstituted C₁₋₃haloalkyl;
n is 1 or 2;
R¹ is R⁷-substituted or unsubstituted indolyl, R⁷-substituted or unsubstituted benzofuranyl, R⁷-substituted or unsubstituted naphthyl, R⁷-substituted or unsubstituted indazolyl, R⁷-substituted or unsubstituted indenyl, R⁷-substituted or unsubstituted benzothiazolyl, R⁷-substituted or unsubstituted isoquinolinyl, R^{7A}-substituted or unsubstituted phenyl, or R^{7A}_substituted or unsubstituted pyridinyl;
each R⁷ is independently hydrogen, halogen, CN, CH₂OH, —OH, NH₂, N(Me)₂, unsubstituted C₁₋₃alkyl, unsubstituted C₂₋₅ alkynyl, unsubstituted C₁₋₃ haloalkyl, or unsubstituted cyclopropyl;
each R^{7a} is independently hydrogen, halogen, NH₂, N(Me)₂, unsubstituted C₁₋₃ alkyl, unsubstituted C₁₋₃haloalkyl, or unsubstituted cyclopropyl;
R² is OH, unsubstituted C₁₋₃alkoxy, unsubstituted C₁₋₃alkyl, or unsubstituted C₁₋₃haloalkyl;
R³ is R⁹-substituted or unsubstituted 4-10 membered heterocycle comprising N, S, or O;
R⁹ is independently halogen, oxo, unsubstituted C₁₋₃ alkyl, unsubstituted C₁₋₃ haloalkyl, unsubstituted C₁₋₃ alkoxy, R¹⁰-substituted or unsubstituted C₁₋₃ alkylidene, or R¹⁰-substituted or unsubstituted C₃₋₄ cycloalkyl, or R¹⁰-substituted or unsubstituted 3 or 4-membered heterocycle;
or wherein two R⁹ together form a C₃₋₅ cycloalkyl or 3-5 membered heterocycle;
R¹⁰ is hydrogen or halogen;
R⁴ is hydrogen, halogen, unsubstituted C₁₋₃alkyl, unsubstituted C₁₋₃alkoxy, or unsubstituted C₁₋₃haloalkyl; and
R⁵ and R⁶ are each independently hydrogen or unsubstituted C₁₋₃alkyl.

2. The compound of claim 1, wherein R¹ is R⁷-substituted or unsubstituted indolyl, R⁷-substituted or unsubstituted benzofuranyl, R⁷-substituted or unsubstituted naphthyl, R⁷-substituted or unsubstituted indazolyl, R⁷-substituted or unsubstituted indenyl, R⁷-substituted or unsubstituted benzothiazolyl, or R⁷-substituted or unsubstituted isoquinolinyl.

3. The compound of claim 1, wherein R¹ is R⁷-substituted or unsubstituted naphthyl or R⁷-substituted or unsubstituted isoquinolinyl.

4. The compound of any one of claim 1, wherein R¹ is

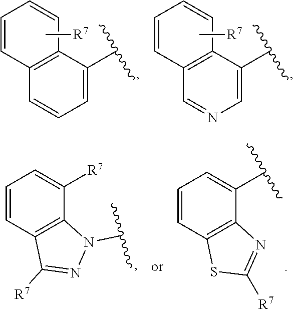

5. The compound of any one of claim 1, wherein R¹ is

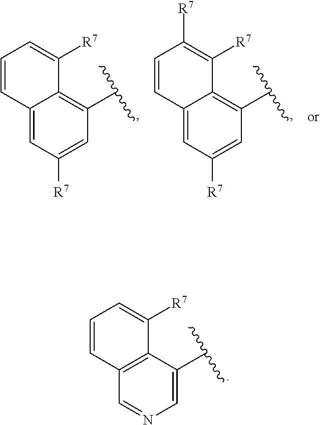

6. The compound of any one of claim 1, wherein R¹ is

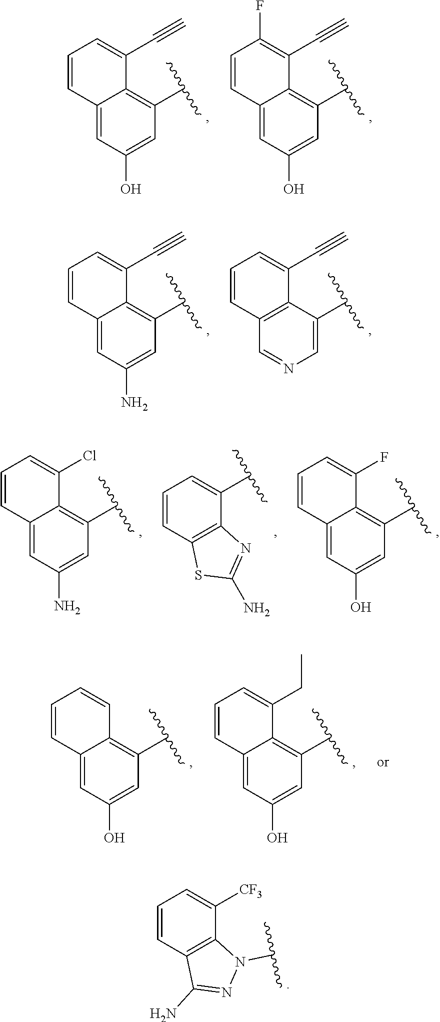

7. The compound of claim 1, wherein R¹ is R^{7A}-substituted or unsubstituted phenyl, or R^{7A}-substituted or unsubstituted pyridinyl.

8. The compound of claim 1, wherein R¹ is 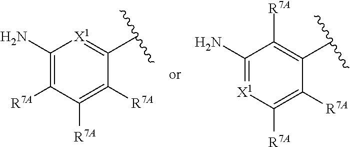
and wherein X¹ is N, CH, CF, or CCl; and
R^{7A} is hydrogen, halogen, unsubstituted C₁₋₃ alkyl, or unsubstituted C₁₋₃ haloalkyl.

9. The compound of claim 1, wherein R¹ is 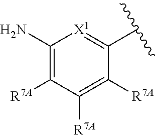
and wherein;
X¹ is N, CH, CF, or CCI; and
R^{7A} is hydrogen, halogen, unsubstituted C₁₋₃ alkyl, or unsubstituted C₁₋₃ haloalkyl.

10. The compound of claim 9, wherein X¹ is N.

11. The compound of claim 1, wherein R¹ is 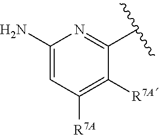
and wherein R^{7A} is hydrogen, halogen, unsubstituted C₁₋₃ alkyl or unsubstituted C₁₋₃ haloalkyl.

12. The compound of claim 1, R¹ is

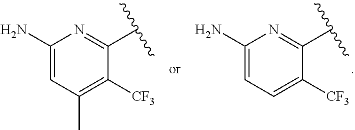 [SMILES: *c1nc(N)cc(C)c1C(F)(F)F ; *c1nc(N)ccc1C(F)(F)F]

13. The compound of claim 9, wherein X¹ is CH, CF, or CCl.

14. The compound of claim 9, wherein R¹ is 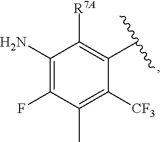
and wherein R^{7A} is hydrogen, F, or Cl.

15. The compound of claim 9, wherein R¹ is 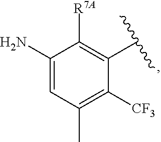
and wherein R^{7A} is hydrogen, F, or Cl.

16. The compound of claim 1, wherein R¹ is 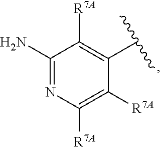
and wherein R^{7A} is hydrogen, halogen, unsubstituted C₁₋₃ alkyl, or unsubstituted C₁₋₃ haloalkyl.

17. The compound of claim 1, wherein R¹ is

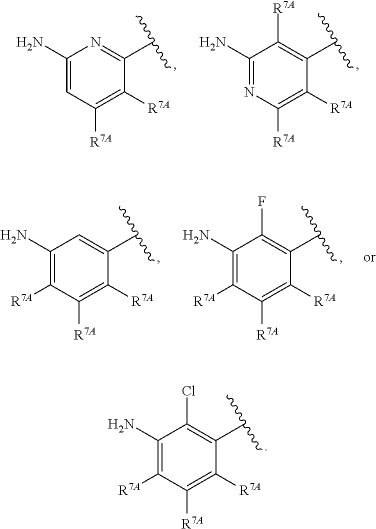

18. The compound of claim 1, wherein R¹ is

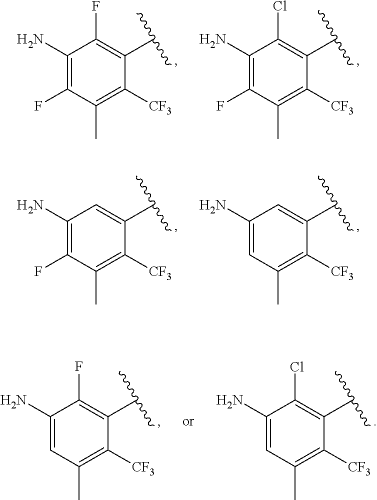 [SMILES: *c1c(F)c(N)c(F)c(C)c1C(F)(F)F ; *c1c(Cl)c(N)c(F)c(C)c1C(F)(F)F ; *c1cc(N)c(F)c(C)c1C(F)(F)F ; *c1cc(N)cc(C)c1C(F)(F)F ; *c1c(F)c(N)cc(C)c1C(F)(F)F ; *c1c(Cl)c(N)cc(C)c1C(F)(F)F]

19. The compound of claim 1, wherein R¹ is

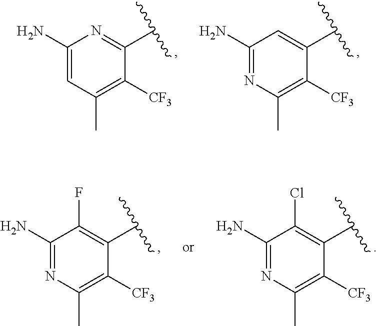 [SMILES: *c1nc(N)cc(C)c1C(F)(F)F ; *c1cc(N)nc(C)c1C(F)(F)F ; *c1c(F)c(N)nc(C)c1C(F)(F)F ; *c1c(Cl)c(N)nc(C)c1C(F)(F)F]

20. The compound of claim 1, wherein R⁴ is Cl or F.

21. The compound of claim 1, wherein R⁴ is F.

22. The compound of claim 1, wherein R⁴ is Cl.

23. The compound of claim 1, wherein R⁴ is OCH₃.

24. The compound of claim 1, wherein R³ is 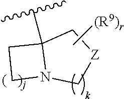
and wherein:
Z is C(R⁹)₂ or O;
R⁹ is hydrogen, halogen or R¹⁰-substituted or unsubstituted C₁₋₃ alkylidene;
or wherein two R⁹ together form a C₃₋₅ cycloalkyl or 3-5 membered heterocycle;
r is an integer of 0-12;
j is 1, 2, or 3; and
k is 1 or 2.

25. The compound of claim 1, wherein R³ is 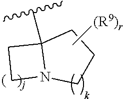
and wherein:
R⁹ is halogen, —OCF₃, —OCHF₂, —OCH₂F, R¹⁰-substituted or unsubstituted C₁₋₃ alkylidene, or two R⁹ together form a R¹⁰-substituted or unsubstituted C₃₋₅ cycloalkyl;
r is an integer of 0-12;
j is 1, 2, or 3; and
k is 1 or 2.

26. The compound of claim 1, wherein R³ is: 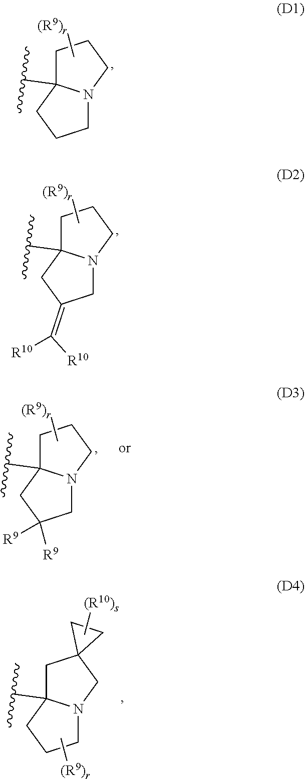
wherein:
each R⁹ is independently halogen or R¹⁰-substituted or unsubstituted C₁₋₃ alkylidene;
each R¹⁰ is independently hydrogen or halogen;
r is 0, 1, or 2; and
s is 0, 1, or 2.

27. The compound of claim 1, wherein R³ is (D1), (D2), or (D3), wherein:
each R⁹ is independently halogen or R¹⁰-substituted or unsubstituted C₁₋₃ alkylidene;
each R¹⁰ is independently hydrogen or halogen;
r is 0, 1, or 2; and
s is 0, 1, or 2.

28. The compound of claim 1, wherein R³ is:

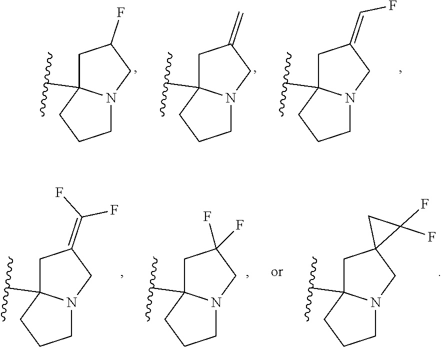

29. The compound of claim 1, wherein R³ is (D1), where r is 1.

30. The compound of claim 1, wherein R³ is (D2) wherein r is 0 and each R¹⁰ is independently hydrogen or fluorine.

31. The compound of claim 1, wherein R³ is (D3) where r is 0 and each R⁹ is independently hydrogen or halogen.

32. The compound of claim 1, wherein R³ is (D4), wherein R¹⁰ is halogen and s is 0, 1, or 2.

33. The compound of claim 1, wherein R³ is 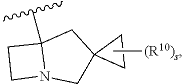
and wherein:
R¹⁰ is halogen, and s is 0, 1, or 2.

34. The compound of claim 1, wherein R³ is 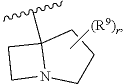
and wherein;
each R⁹ is independently halogen, oxo, or unsubstituted C₁₋₃ alkyl; and
r is 0, 1, or 2.

35. The compound of claim 1, wherein R³ is 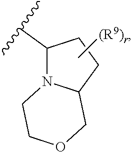
and wherein:
each R⁹ is independently halogen, oxo, or unsubstituted C₁₋₃ alkyl; and
r is 0, 1, or 2.

36. The compound of claim 1, wherein R³ is 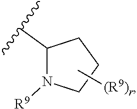
and wherein:
each R⁹ is independently halogen, oxo, or unsubstituted C₁₋₃ alkyl;
or
two R⁹ together form a C₃₋₅ cycloalkyl or 3-5 membered heterocycle; and
r is 1 or 2.

37. The compound of claim 1, wherein R³ is

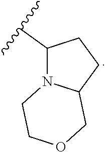 [SMILES: *C1CCC2COCCN12]

38. The compound of claim 1, wherein R³ is

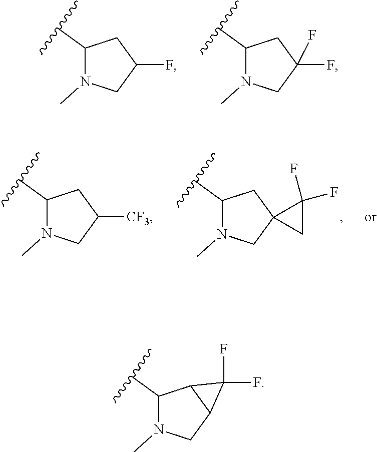

39. The compound of claim 1, wherein R² is methyl.

40. The compound of claim 1, wherein R² is CHF₂, CH₂F, or CF₃.

41. The compound of claim 1, wherein Ring A is R⁸-substituted or unsubstituted 3-10 membered heterocycle, or R⁸-substituted or unsubstituted 5-10 membered heteroaryl.

42. The compound of claim 1, wherein Ring A is R⁸-substituted or unsubstituted 3-6 membered heterocycle, or R⁸-substituted or unsubstituted 5-6 membered heteroaryl.

43. The compound of claim 1, wherein Ring A is a R⁸-substituted or unsubstituted 3-6 membered heterocycle comprising at least 1 heterocyclic ring nitrogen atom, or R⁸-substituted or unsubstituted 5-6 membered heteroaryl comprising at least 1 heterocyclic ring nitrogen atom.

44. The compound of claim 1, wherein Ring A is a R⁸-substituted or unsubstituted 5 or 6 membered heterocycle comprising at least 1 heterocyclic ring nitrogen atom.

45. The compound of claim 1, wherein Ring A is a R⁸-substituted or unsubstituted 6 membered heteroaryl comprising at least 1 heterocyclic ring nitrogen atom.

46. The compound of claim 1, wherein Ring A is a R⁸-substituted or unsubstituted pyridinyl, R⁸-substituted or unsubstituted pyrimidinyl or R⁸-substituted or unsubstituted pyrazinyl.

47. The compound of claim 1, wherein Ring A is a moiety of formula; 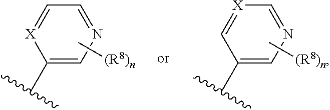
wherein X is CH or N and n is 1 or 2.

48. The compound of claim 1, wherein Ring A is a moiety of formula; 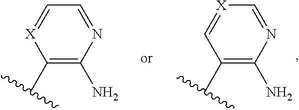
wherein X is CH or N.

49. The compound of claim 1, wherein R⁵ and R⁶ are each hydrogen.

50. The compound of claim 1, wherein R⁵ is CH₃ and R⁶ is hydrogen.

51. A compound of Table 1 or a stereoisomer, atropisomer, tautomer, or pharmaceutically acceptable salt thereof.

52. A compound of Table 2 or a stereoisomer, atropisomer, tautomer, or pharmaceutically acceptable salt thereof.

53. A compound of Table 3 or a stereoisomer, atropisomer, tautomer, or pharmaceutically acceptable salt thereof.

54. A pharmaceutical composition comprising a compound or a stereoisomer, atropisomer, tautomer, or pharmaceutically acceptable salt thereof of claim 1 and one or more pharmaceutically acceptable excipients.

55. A method of treating cancer, the method comprising administering an effective amount of a compound or a stereoisomer, atropisomer, tautomer, or pharmaceutically acceptable salt thereof of claim 1.

56. A method of treating cancer, the method comprising administering an effective amount of a compound or a stereoisomer, atropisomer, tautomer, or pharmaceutically acceptable salt thereof of claim 1.

57. The method of claim 56, wherein the cancer is characterized as comprising a KRas mutation.

58. The method of claim 57, wherein the KRas mutation corresponds to a KRas^{G12D} mutation or KRas^{G12V} mutation.

59. The method of claim 57, further comprising testing a sample from the patient before administration for the absence or presence of a KRas mutation.

60. The method of claim 59, wherein the compound, stereoisomer, atropisomer, tautomer, or pharmaceutically acceptable salt thereof or pharmaceutical composition is administered to the patient after the patient sample shows the presence of a KRas mutation.

61. The method of claim 56, wherein the cancer is tissue agnostic.

62. The method of claim 56, wherein the cancer is pancreatic cancer, lung cancer, or colorectal cancer.

63. The method of claim 62, wherein the lung cancer is lung adenocarcinoma, NSCLC, or SCLC.

64. The method of claim 62, wherein the cancer is pancreatic cancer.

65. The method of claim 62, wherein the cancer is colorectal cancer.

66. The method of any one of claim 56, further comprising administering at least one additional therapeutic agent.

67. The method of claim 66, wherein the additional therapeutic agent comprises an epidermal growth factor receptor (EGFR) inhibitor, phosphatidylinositol kinase (PI3K) inhibitor, insulin-like growth factor receptor (IGF1R) inhibitor, a Janus kinase (JAK) inhibitor, a Met kinase inhibitor, a SRC family kinase inhibitor, a mitogen-activated protein kinase (MEK) inhibitor, an extracellular-signal-regulated kinase (ERK) inhibitor, a topoisomerase inhibitor, a taxane, an anti-metabolite agent, or an alkylating agent.

68. (canceled)

69. (canceled)

70. (canceled)

71. (canceled)

72. (canceled)

73. A method for regulating activity of a KRas mutant protein, the method comprising reacting the mutant protein with a compound of claim 1, or stereoisomer, atropisomer, tautomer, or pharmaceutically acceptable salt thereof.

74. A method for inhibiting proliferation of a cell population, the method comprising contacting the cell population with the compound of claim 1, or stereoisomer, atropisomer, tautomer, or pharmaceutically acceptable salt thereof.

75. The method of claim 74, wherein the inhibition of proliferation is measured as a decrease in cell viability of the cell population.

76. A method for preparing a labeled KRas mutant protein, the method comprising reacting a KRas mutant protein with a labeled compound of claim 1, or stereoisomer, atropisomer, tautomer, or pharmaceutically acceptable salt thereof, to result in the labeled KRas mutant protein.

77. A method for inhibiting tumor metastasis, the method comprising: administering to an individual in need thereof a therapeutically effective amount of the compound of claim 1, or stereoisomer, atropisomer, tautomer, or pharmaceutically acceptable salt thereof.

78. (canceled)
